(19) United States
(12) Patent Application Publication
(10) Pub. No.: US 2013/0217664 A1
(43) Pub. Date: Aug. 22, 2013

(54) NOVEL COMPOUNDS USEFUL FOR THE TREATMENT OF DEGENERATIVE AND INFLAMMATORY DISEASES

(71) Applicants: Christel Jeanne Marie MENET, Mechelen (BE); Benoit Antoine SCHMITT, Mechelen (BE); Raphael Jean Joel GENEY, Romainville (FR); Kevin James DOYLE, Essex (GB); Joanne PEACH, Essex (GB); Nicholas John PALMER, Essex (GB); Graham Peter JONES, Essex (GB); David HARDY, Essex (GB); James Edward Stewart DUFFY, Essex (GB)

(72) Inventors: Christel Jeanne Marie MENET, Mechelen (BE); Benoit Antoine SCHMITT, Mechelen (BE); Raphael Jean Joel GENEY, Romainville (FR); Kevin James DOYLE, Essex (GB); Joanne PEACH, Essex (GB); Nicholas John PALMER, Essex (GB); Graham Peter JONES, Essex (GB); David HARDY, Essex (GB); James Edward Stewart DUFFY, Essex (GB)

(21) Appl. No.: 13/762,155

(22) Filed: Feb. 7, 2013

Related U.S. Application Data
(60) Provisional application No. 61/597,707, filed on Feb. 10, 2012.
(60) Provisional application No. 61/753,482, filed on Jan. 17, 2013.

(51) Int. Cl. C07D 471/04; A61K 31/506; A61K 31/496; A61K 31/541; A61K 31/4545; A61K 31/5377; A61K 31/497; A61K 45/06; A61K 31/444; A61K 31/437; C07D 491/107

(52) U.S. Cl. CPC C07D 471/04; A61K 31/437; A61K 31/506; A61K 31/496; A61K 31/541; C07D 491/107; A61K 31/5377; A61K 31/497; A61K 45/06; A61K 31/444; A61K 31/4545
USPC 514/210.18; 546/118; 514/303; 514/256; 544/333; 514/253.04; 544/362; 514/228.5; 544/58.2; 546/15; 514/210.21; 514/234.2; 544/127; 544/316; 514/274; 514/275; 544/331; 544/405; 514/255.05; 544/322

(57) ABSTRACT

Novel imidazolopyridines according to Formula I, able to inhibit JAK are disclosed, these compounds may be prepared as a pharmaceutical composition, and may be used for the prevention and treatment of a variety of conditions in mammals including humans, including by way of non-limiting example, allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.

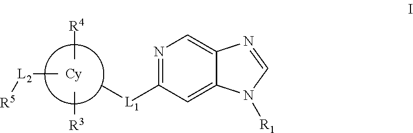

Wherein R¹, L₁, R³, R⁴, Cy, L₂ and R⁵ are as defined herein.

### CROSS REFERENCE TO RELATED APPLICATIONS

The present application claims the priority of co-pending provisional application U.S. Ser. No. 61/597,707 filed on Feb. 10, 2012, and Ser. No. 61/753,482 filed on Jan. 17, 2013, and the disclosures of both applications are incorporated by reference herein in their entireties. Applicants claim the benefits of both applications under 35 U.S.C. §119(e).

### FIELD OF THE INVENTION

The present invention relates to compounds that are inhibitors of JAK, a family of tyrosine kinases that are involved in allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons. In particular, the compounds of the invention inhibit JAK1 and/or JAK2. The present invention also provides methods for the production of the compounds of the invention, pharmaceutical compositions comprising the compounds of the invention, methods for the prevention and/or treatment of diseases involving allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons by administering a compound of the invention.

Janus kinases (JAKs) are cytoplasmic tyrosine kinases that transduce cytokine signaling from membrane receptors to STAT transcription factors. Four JAK family members are described, JAK1, JAK2, JAK3 and TYK2. Upon binding of the cytokine to its receptor, JAK family members auto- and/or transphosphorylate each other, followed by phosphorylation of STATs that then migrate to the nucleus to modulate transcription. JAK-STAT intracellular signal transduction serves the interferons, most interleukins, as well as a variety of cytokines and endocrine factors such as EPO, TPO, GH, OSM, LIF, CNTF, GM-CSF and PRL (Vainchenker W. et al. (2008)).

The combination of genetic models and small molecule JAK inhibitor research revealed the therapeutic potertial of several JAKs. JAK3 is validated by mouse and human genetics as an immune-suppression target (O'Shea J. et al. (2004)). JAK3 inhibitors were successfully taken into clinical development, initially for organ transplant rejection but later also in other immuno-inflammatory indications such as rheumathoid arthritis (RA), psoriasis and Crohn's disease (http://clinicaltrials.gov/).

TYK2 is a potertial target for immuno-inflammatory diseases, being validated by human genetics and mouse knock-out studies (Levy D. and Loomis C. (2007)).

JAK1 is a target in the immuno-inflammatory disease area. JAK1 heterodimerizes with the other JAKs to transduce cytokine-driven pro-inflammatory signaling. Therefore, inhibition of JAK1 is of interest for immuno-inflammatory diseases with pathology-associated cytokines that use JAK1 signaling, such as IL-6, IL-4, IL-5, IL-12, IL-13, IL-23, or IFNgamma, as well as for other diseases driven by JAK-mediated signal transduction.

### BACKGROUND OF THE INVENTION

The degeneration of cartilage is the hallmark of various diseases, among which rheumatoid arthritis and osteoarthritis are the most prominent. Rheumatoid arthritis (RA) is a chronic joint degenerative disease, characterized by inflammation and destruction of the joint structures. When the disease is unchecked, it leads to substantial disability and pain due to loss of joint functionality and even premature death. The aim of an RA therapy, therefore, is not only to slow down the disease but to attain remission in order to stop the joint destruction. Besides the severity of the disease outcome, the high prevalence of RA (˜0.8% of adults are affected worldwide) means a high socio-economic impact. (For reviews on RA, we refer to Smolen and Steiner (2003); Lee and Weinblatt (2001); Choy and Panayi (2001); O'Dell (2004) and Firestein (2003)).

JAK1 and JAK2 are implicated in intracellular signal transduction for many cytokines and hormones. Pathologies associated with any of these cytokines and hormones can be ameliorated by JAK1 and JAK2 inhibitors. Hence, several allergy, inflammation and autoimmune disorders might benefit from treatment with compounds described in this invention including rheumatoid arthritis, systemic lupus erythematosis, juvenile idiopathic arthritis, osteoarthritis, asthma, chronic obstructive pulmonary disease COPD, tissue fibrosis, eosinophilic inflammation, eosophagitis, inflammatory bowel diseases (e.g. Crohn's, ulcerative colitis), transplantation, graft-versus-host disease, psoriasis, myositis, multiple sclerosis (Kopf et al., 2010).

Osteoarthritis (also referred to as OA, or wear-and-tear arthritis) is the most common form of arthritis and is characterized by loss of articular cartilage, often associated with hypertrophy of the bone and pain. For an extensive review on osteoarthritis, we refer to Wieland et al. (2005).

Osteoarthritis is difficult to treat. At present, no cure is available and treatment focuses on relieving pain and preventing the affected joint from becoming deformed. Common treatments include the use of non-steroidal anti-inflammatory drugs (NSAIDs). Although dietary supplements such as chondroitin and glucosamine sulphate have been advocated as safe and effective options for the treatment of osteoarthritis, a recent clinical trial revealed that both treatments did not reduce pain associated with osteoarthritis. (Clegg et al., 2006).

Stimulation of the anabolic processes, blocking catabolic processes, or a combination of these two, may result in stabilization of the cartilage, and perhaps even reversal of the damage, and therefore prevent further progression of the disease. Therapeutic methods for the correction of the articular cartilage lesions that appear during the osteoarthritic disease have been developed, but so far none of them have been able to mediate the regeneration of articular cartilage in situ and in vivo. Taken together, no disease modifying osteoarthritic drugs are available.

Vandeghinste et al. (WO 2005/124342) discovered JAK1 as a target whose inhibition might have therapeutic relevance for several diseases including OA. Knockout of the JAK1 gene in mice demonstrated that JAK1 plays essential and non-redundant roles during development: JAK1−/− mice died within 24 h after birth and lymphocyte development was severely impaired. Moreover, JAK1−/− cells were not, or less, reactive to cytokines that use class II cytokine receptors, cytokine receptors that use the gamma-c subunit for signaling and the family of cytokine receptors that use the gp130 subunit for signaling (Rodig et al., 1998).

Various groups have implicated JAK-STAT signaling in chondrocyte biology. Li et al. (2001) showed that Oncostatin M induces MMP and TIMP3 gene expression in primary chondrocytes by activation of JAK/STAT and MAPK signaling pathways. Osaki et al. (2003) showed that interferon-gamma mediated inhibition of collagen II in chondrocytes involves JAK-STAT signaling. IL1-beta induces cartilage catabolism by reducing the expression of matrix components, and by inducing the expression of collagenases and inducible nitric oxide synthase (NOS2), which mediates the production of nitric oxide (NO). Otero et al., (2005) showed that leptin and IL1-beta synergistically induced NO production or expression of NOS2 mRNA in chondrocytes, and that that was blocked by a JAK inhibitor. Legendre et al. (2003) showed that IL6/IL6Receptor induced downregulation of cartilage-specific matrix genes collagen II, aggrecan core and link protein in bovine articular chondrocytes, and that this was mediated by JAK/STAT signaling. Therefore, these observations suggest a role for JAK kinase activity in cartilage homeostasis and therapeutic opportunities for JAK kinase inhibitors.

JAK family members have been implicated in additional conditions including myeloproliferative disorders (O'Sullivan et al, 2007, Mol Immunol 44(10):2497-506), where mutations in JAK2 have been identified. This indicates that inhibitors of JAK in particular JAK2 may also be of use in the treatment of myeloproliferative disorders. Additionally, the JAK family, in particular JAK1, JAK2 and JAK3, has been linked to cancers, in particular leukaemias e.g. acute myeloid leukaemia (O'Sullivan et al, 2007, Mol Immunol 44(10):2497-506; Xiang et al., 2008, “Identification of somatic JAK1 mutations in patients with acute myeloid leukemia” Blood First Edition Paper, prepublished online Dec. 26, 2007; DOI 10.1182/blood-2007-05-090308) and acute lymphoblastic leukaemia (Mullighan et al, 2009) or solid tumours e.g. uterine leiomyosarcoma (Constantinescu et al., 2007, Trends in Biochemical Sciences 33(3): 122-131), prostate cancer (Tam et al., 2007, British Journal of Cancer, 97, 378-383). These results indicate that inhibitors of JAK, in particular of JAK1 and/or JAK2, may also have utility in the treatment of cancers (leukaemias and solid tumours e.g. uterine leiomyosarcoma, prostate cancer).

In addition, Castleman's disease, multiple myeloma, mesangial proliferative glomerulonephritis, psoriasis, and Kaposi's sarcoma are likely due to hypersecretion of the cytokine IL-6, whose biological effects are mediated by intracellular JAK-STAT signaling (Tetsuji Naka, Norihiro Nishimoto and Tadamitsu Kishimoto, Arthritis Res 2002, 4 (suppl 3):5233-5242). This result shows that inhibitors of JAK, may also find utility in the treatment of said diseases.

The current therapies are not satisfactory and therefore there remains a need to identify further compounds that may be of use in the treatment of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons. The present invention therefore provides compounds, methods for their manufacture and pharmaceutical compositions comprising the compounds of the invention together with a suitable pharmaceutical carrier. The present invention also provides for the use of a compound of the invention in the preparation of a medicament for the treatment of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.

### SUMMARY OF THE INVENTION

The present invention is based on the discovery that the compounds of the invention are able to act as inhibitors of JAK and that they are useful for the treatment of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons. In a specific aspect the compounds of the invention are inhibitors of JAK1 and/or JAK2. The present invention also provides methods for the production of these compounds, pharmaceutical compositions comprising these compounds and methods for treating allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons by administering the compounds of the invention.

Accordingly, in a first aspect of the invention, the compounds of the invention are provided having a Formula I:

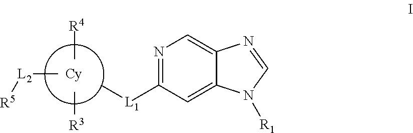

wherein
R¹ is Me, Et, or cyclopropyl, each of which is optionally substituted with one or more halo;

### L₁ is —NR²—; —O—, or —CH₂—;

Cy is phenyl, or 5-9 membered heteroaryl comprising 1 to 4 heteroatoms independently selected from N, O, and S;

### R² is

C₁₋₄ alkyl optionally substituted with one or more groups independently selected from
   C₃₋₇ cycloalkyl,
   halo,
   CN,
   NR^{15a}R^{15b} wherein each R^{15a} and R^{15b} is independently selected from C₁₋₄ alkyl,
   4-6 membered heterocycloalkyl containing 1 to 2 heteroatoms independently selected from N, O, and S, and
   C₁₋₄ alkoxy, or
C₃₋₇ cycloalkyl;

### R³ is

—H,
halo,
cyclopropyl,
C₁₋₄ alkyl optionally substituted with one or more halo, or
C₁₋₄ alkoxy optionally substituted with one or more halo;
   R⁴ is H, or halo;

### L₂ is

absent, or is
—W—,
C₁₋₂ alkylene- (wherein the alkylene is optionally substituted with one CN),
C₁₋₂ alkylene-W— (wherein the alkylene is optionally substituted with one CN), or
—CH═CH—;
   W is —C(═O)—, —C(═O)O—, —C(═O)NR⁶—, —NR⁶C(═O)—, —NR⁶C(═O)O—, —NR⁶C(═O)NH—, —S—, —SO₂—, —SO₂NR⁶—, —NR⁶SO₂—, —NHSO₂NR⁶—, —O—, or NR⁶;

### R⁵ is

—H,
CN,
C₁₋₆ alkyl optionally substituted with one or more independently selected R⁷ groups,
C₃₋₇ cycloalkyl, optionally substituted with one or more groups independently selected from R¹⁰,
4-7 membered heterocycloalkyl comprising 1 or 2 heteroatoms independently selected from N, O, and S, optionally substituted with one or more groups independently selected from R¹⁰,
4-7 membered heterocycloalkenyl comprising 1 double bond, and comprising 1 or 2 heteroatoms independently selected from N, O, and S, optionally substituted with one or more groups independently selected from R¹⁰,
C₆₋₁₀ aryl optionally substituted with one or more groups independently selected from R¹¹, or
5-6 membered heteroaryl comprising 1 to 4 heteroatoms independently selected from N, O, and S, optionally substituted with one or more groups independently selected from R¹¹;
   R⁶ is H, or C₁₋₄ alkyl optionally substituted with CN, C₁₋₂ alkoxy, or C₃₋₆ cycloalkyl;
R⁷ is
—OH,
CN,
halo,
C₁₋₄ alkoxy,
4-7 membered heterocycloalkyl comprising 1 or 2 heteroatoms independently selected from N, O, and S, optionally substituted with one or more groups independently selected from halo, C₁₋₄ alkyl and oxo,
NR^{8a}R^{8b},
5-6 membered heteroaryl comprising 1 to 4 heteroatoms independently selected from N, O, and S, optionally substituted with one or more independently selected C₁₋₄ alkyl, CN, halo, and C₁₋₄ alkoxy,
phenyl optionally substituted with one or more groups independently selected from C₁₋₄ alkyl, CN, halo, and C₁₋₄ alkoxy,
C₃₋₇ cycloalkyl,
—C(═O)NR^{9a}R^{9b},
—OSO₂C₁₋₄ alkyl (which alkyl is optionally substituted with one or more halo), or
—NR^{9c}SO₂C₁₋₄ alkyl (which alkyl is optionally substituted with one or more halo);
   each R^{8a}, and R^{8b} is independently selected from H, and C₁₋₄ alkyl;
   each R^{9a}, R^{9b} and R^{9c} is independently selected from H, and C₁₋₄ alkyl;
   each R¹⁰ is independently selected from oxo and R¹¹;
   each R¹¹ is halo, —CN or L₃-R¹²;
   L₃ is absent or is —C(═O)—, C(═O)O—, —O—, SO₂—, —C(═O)NR^{13a}, —NR^{13b}C(═O), or NR^{13c};
   each R¹² is
—H,
C₁₋₄ alkyl optionally substituted with one or more groups independently selected from:
halo,
   OH,
   CN,
   C₁₋₄ alkoxy,
   NHC(═O)O—C₁₋₄ alkyl,
   —C(═O)NR^{14a}R^{14b},
   —NR^{14c}R^{14d},
   —C(═O)C₁₋₄ alkyl,
   —C(═O)O—C₁₋₄ alkyl,
   phenyl optionally substituted with halo, C₁₋₄ alkyl, C₁₋₄ alkoxy, and
   4-7 membered heterocycloalkyl comprising 1 or 2 heteroatoms independently selected from N, O, and S, optionally substituted with one or more C₁₋₄ alkyl;
4-7 membered heterocycloalkyl comprising 1 or 2 heteroatoms independently selected from N, O, and S, optionally substituted with one or more groups independently selected from C₁₋₄ alkyl, oxo and CN
5-6 membered heteroaryl comprising 1 to 4 heteroatoms independently selected from N, O, and S, optionally substituted with one or more independently selected C₁₋₄ alkyl, or
C₃₋₇ cycloalkyl optionally substituted with one or more groups independently selected from OH, halo, C₁₋₄ alkyl, and CN; and
   each R^{13a}, R^{13b}, R^{13c}, R^{14a}, R^{14b}, R^{14c}, and R^{14d}, is independently selected from H, and C₁₋₄ alkyl; provided that R³, R⁴, and -L₂-R⁵ are not all simultaneously H when Cy is C₆ aryl, or 6-membered heteroaryl.

In a particular embodiment the compounds of the invention are inhibitors of JAK1 and/or JAK2.

In a further aspect, the present invention provides pharmaceutical compositions comprising the compounds of the invention, and a pharmaceutical carrier, excipient or diluent. Moreover, the compounds of the invention, useful in the pharmaceutical compositions and treatment methods disclosed herein, are pharmaceutically acceptable as prepared and used. In this aspect of the invention, the pharmaceutical composition may additionally comprise further active ingredients suitable for use in combination with the compounds of the invention.

In a further aspect, the invention provides a compound of the invention or a pharmaceutical composition comprising a compound of the invention for use as a medicament. In a specific embodiment, said pharmaceutical composition additionally comprises a further active ingredient.

In a further aspect of the invention, this invention provides a method of treating a mammal susceptible to or afflicted with a condition from among those listed herein, and particularly, such condition as may be associated with aberrant JAK activity, e.g. allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons, which method comprises administering an effective amount of the pharmaceutical composition or compound of the invention as described herein. In a specific embodiment the condition is associated with aberrant JAK1 and/or JAK2 activity.

In a further aspect, the present invention provides the compounds of the invention for use in the treatment or prophylaxis of a condition selected from those listed herein, particularly such conditions as may be associated with aberrant JAK activity, e.g. allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.

In yet another method of treatment aspect, this invention provides a method for treating a mammal susceptible to or afflicted with a condition that is causally related to abnormal JAK activity as described herein, and comprises administering an effective condition-treating or condition-preventing amount of the pharmaceutical composition or a compound of the invention described herein. In a specific aspect the condition is causally related to abnormal JAK1 and/or JAK2 activity.

In a further aspect, the present invention provides a compound of the invention, or a pharmaceutical composition comprising a compound of the invention, for use as a medicament.

In a further aspect, the present invention provides the compounds of the invention for use in the treatment or prophylaxis of a condition that is causally related to abnormal JAK activity.

In additional aspects, this invention provides methods for synthesizing the compounds of the invention, with representative synthetic protocols and pathways disclosed later on herein.

Accordingly, it is a principal object of this invention to provide novel compounds, which can modify the activity of JAK and thus prevent or treat any conditions that may be causally related thereto. In a specific aspect the compounds of the invention modulate the activity of JAK1 and/or JAK2.

It is a further object of this invention to provide compounds that can treat or alleviate conditions or symptoms of same, such as allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons, that may be causally related to the activity of JAK, in particular JAK1 and/or JAK2.

A still further object of this invention is to provide a pharmaceutical composition that may be used in the treatment or prophylaxis of a variety of conditions, including the diseases associated with JAK activity such as allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons. In a specific embodiment the disease is associated with JAK1 and/or JAK2 activity.

Other objects and advantages will become apparent to those skilled in the art from a consideration of the ensuing detailed description.

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles “a” and “an” may be used herein to refer to one or to more than one (i.e. at least one) of the grammatical objects of the article. By way of example “an analogue” means one analogue or more than one analogue.

As used herein the term ‘JAK’ relates to the family of Janus kinases (JAKs) which are cytoplasmic tyrosine kinases that transduce cytokine signaling from membrane receptors to STAT transcription factors. Four JAK family members are described, JAK1, JAK2, JAK3 and TYK2 and the term JAK may refer to all the JAK family members collectively or one or more of the JAK family members as the context indicates.

‘Alkoxy’ refers to the group OR²⁶ where R²⁶ is alkyl with the number of carbon atoms specified. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

‘Alkylene’ refers to divalent alkene radical groups having the number of carbon atoms specified, in particular having 1 to 6 carbon atoms and more particularly 1 to 4 carbon atoms which can be straight-chained or branched. This term is exemplified by groups such as methylene (—CH₂—), ethylene (—CH₂—CH₂—), or —CH(CH₃)— and the like.

‘Alkyl’ means straight or branched aliphatic hydrocarbon with the number of carbon atoms specified. Particular alkyl groups have 1 to 8 carbon atoms. More particular is lower alkyl which has 1 to 6 carbon atoms. A further particular group has 1 to 4 carbon atoms. Exemplary straight chained groups include methyl, ethyl n-propyl, and n-butyl. Branched means that one or more lower alkyl groups such as methyl, ethyl, propyl or butyl is attached to a linear alkyl chain, exemplary branched chain groups include isopropyl, iso-butyl, t-butyl and isoamyl.

‘Alkenyl’ refers to monovalent olefinically (unsaturated) hydrocarbon groups with the number of carbon atoms specified. Particular alkenyl has 2 to 8 carbon atoms, and more particularly, from 2 to 6 carbon atoms, which can be straight-chained or branched and having at least 1 and particularly from 1 to 2 sites of olefinic unsaturation. Particular alkenyl groups include ethenyl (—CH═CH₂), n-propenyl (—CH₂CH═CH₂), isopropenyl (—C(CH₃)═CH₂) and the like.

‘Amino’ refers to the radical —NH₂.

‘Aryl’ refers to a monovalent aromatic hydrocarbon group derived by the removal of one hydrogen atom from a single carbon atom of a parent aromatic ring system. In particular aryl refers to an aromatic ring structure, monocyclic or polyyclic, with the number of ring atoms specified. Specifically, the term includes groups that include from 6 to 10 ring members. Where the aryl group is a monocyclic ring system it preferentially contains 6 carbon atoms. Particularly aryl groups include phenyl, naphthyl, indenyl, and tetrahydronaphthyl.

‘Cycloalkyl’ refers to a non-aromatic hydrocarbyl ring structure, monocyclic or polycyclic, with the number of ring atoms specified. A cycloalkyl may have from 3 to 10 carbon atoms, and in particular from 3 to 7 carbon atoms. Such cycloalkyl groups include, by way of example, single ring structures such as cyclopropyl, cyclobutyl, cyclopentyl, cyclohexyl, and cycloheptyl.

‘Cyano’ refers to the radical —CN.

‘Halo’ or ‘halogen’ refers to fluoro (F), chloro (Cl), bromo (Br) and iodo (I). Particular halo groups are either fluoro or chloro.

‘Hetero’ when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. heterocycloalkyl, aryl, e.g. heteroaryl, and the like having from 1 to 4, and particularly from 1 to 3 heteroatoms, more typically 1 or 2 heteroatoms, for example a single heteroatom.

‘Heteroaryl’ means an aromatic ring structure, monocyclic or polycyclic, that includes one or more heteroatoms independently selected from O, N and S and the number of ring atoms specified. In particular, the aromatic ring structure may have from 5 to 10 ring members. The heteroaryl group can be, for example, a five membered or six membered monocyclic ring or a bicyclic structure formed from fused five and six membered rings or two fused six membered rings or, by way of a further example, two fused five membered rings. Each ring may contain up to four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five. Examples of five membered monocyclic heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups. Examples of six membered monocyclic heteroaryl groups include but are not limited to pyridine, pyrazine, pyridazine, pyrimidine and triazine. Particular examples of bicyclic heteroaryl groups containing a five membered ring fused to another five membered ring include but are not limited to imidazothiazole and imidazoimidazole. Particular examples of bicyclic heteroaryl groups containing a six membered ring fused to a five membered ring include but are not limited to benzfuran, benzthiophene, benzimidazole, benzoxazole, is obenzoxazole, benzisoxazole, benzthiazole, benzisothiazole, isobenzofuran, indole, isoindole, isoindolone, indolizine, indoline, isoindoline, purine (e.g. adenine, guanine), indazole, pyrazolopyrimidine, triazolopyrimidine, benzodioxole and pyrazolopyridine groups. Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups. Particular heteroaryl groups are those derived from thiophene, pyrrole, benzothiophene, benzofuran, indole, pyridine, quinoline, imidazole, oxazole and pyrazine.

Examples of representative heteroaryls include the following:

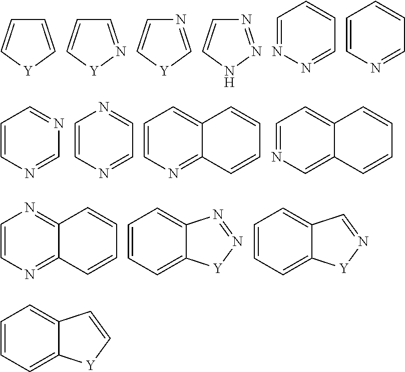

wherein each Y is selected from >C═O, NH, O and S.

As used herein, the term ‘heterocycloalkyl’ means a stable non-aromatic ring structure, mono-cyclic or polycyclic, that includes one or more heteroatoms independently selected from O, N and S and the number of ring atoms specified. The non-aromatic ring structure may have from 4 to 10 ring members, and in particular from 4 to 7 ring members. A fused heterocyclic ring system may include carbocyclic rings and need only to include one heterocyclic ring. Examples of heterocyclic rings include, but are not limited to, morpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone, pyran, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. Further examples include thiomorpholine and its S-oxide and S,S-dioxide (particularly thiomorpholine). Still further examples include azetidine, piperidone, piperazone, and N-alkyl piperidines such as N-methyl piperidine. Particular examples of heterocycloalkyl groups are shown in the following illustrative examples:

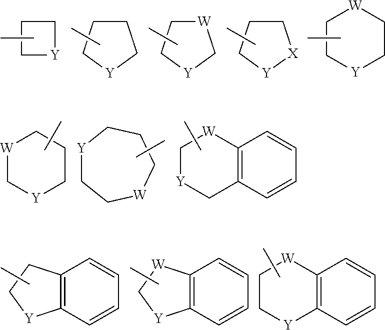

wherein each W is selected from CH₂, NH, O and S; and each Y is selected from NH, O, CO, SO₂, and S.

As used herein, the term ‘heterocycloalkenyl’ means a ‘heterocycloalkyl, wherein one bond of the ring is reduced, thus the ring comprises a double bond. Particular examples of heterocycloalkenyl groups are shown in the following illustrative examples:

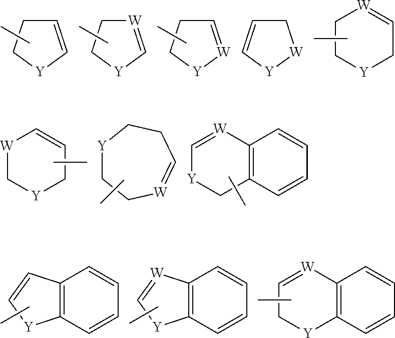

wherein each W is selected from CH₂, NH, O and S; and each Y is selected from NH, O, CO, SO₂, and S.

‘Hydroxyl’ refers to the radical —OH.

‘Substituted’ refers to a group in which one or more hydrogen atoms are each independently replaced with the same or different substituent(s).

‘Sulfo’ or ‘sulfonic acid’ refers to a radical such as SO₃H.

‘Thiol’ refers to the group —SH.

As used herein, term ‘substituted with one or more’ refers to one to four substituents. In one embodiment it refers to one to three substituents. In further embodiments it refers to one or two substituents. In a yet further embodiment it refers to one substituent.

‘Thioalkoxy’ refers to the group SR²⁶ where R²⁶ has the number of carbon atoms specified and particularly C₁-C₈ alkyl. Particular thioalkoxy groups are thiomethoxy, thioethoxy, n-thiopropoxy, isothiopropoxy, n-thiobutoxy, tert-thiobutoxy, sec-thiobutoxy, n-thiopentoxy, n-thiohexoxy, and 1,2-dimethylthiobutoxy. Particular thioalkoxy groups are lower thioalkoxy, i.e. with between 1 and 6 carbon atoms. Further particular alkoxy groups have between 1 and 4 carbon atoms.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

‘Prodrugs’ refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

‘Solvate’ refers to forms of the compound that are associated with a solvent, usually by a solvolysis reaction. This physical association includes hydrogen bonding Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans. The terms ‘human’, ‘patient’ and ‘subject’ are used interchangeably herein.

‘Therapeutically effective amount’ means the amount of a compound that, when administered to a subject for treating a disease, is sufficient to effect such treatment for the disease. The ‘therapeutically effective amount’ can vary depending on the compound, the disease and its severity, and the age, weight, etc., of the subject to be treated.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e. arresting the disease or reducing the manifestation, extert or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g. stabilization of a discernible symptom), physiologically, (e.g. stabilization of a physical parameter), or both. In a further embodiment, “treating” or “treatment” relates to slowing the progression of the disease.

As used herein the term ‘allergy’ refers to the group of conditions characterized by a hypersensitivity disorder of the immune system including, allergic airway disease (e.g. asthma, rhinitis), sinusitis, eczema and hives, as well as food allergies or allergies to insect venom.

As used herein the term ‘inflammatory condition(s)’ refers to the group of conditions including, rheumatoid arthritis, osteoarthritis, juvenile idiopathic arthritis, psoriasis, psoriatic arthritis, allergic airway disease (e.g. asthma, rhinitis), inflammatory bowel diseases (e.g. Crohn's disease, ulcerative colitis), endotoxin-driven disease states (e.g. complications after bypass surgery or chronic endotoxin states contributing to e.g. chronic cardiac failure), and related diseases involving cartilage, such as that of the joints. Particularly the term refers to rheumatoid arthritis, osteoarthritis, allergic airway disease (e.g. asthma) and inflammatory bowel diseases.

As used herein the term ‘autoimmune disease(s)’ refers to the group of diseases including obstructive airways disease, including conditions such as COPD, asthma (e.g intrinsic asthma, extrinsic asthma, dust asthma, infantily asthma) particularly chronic or inveterate asthma (for example late asthma and airway hyperreponsiveness), bronchitis, including bronchial asthma, systemic lupus erythematosus (SLE), cutaneous lupus erythrematosis, lupus nephritis, dermatomyositis, Sjogren's syndrome, multiple sclerosis, psoriasis, dry eye disease, type I diabetes mellitus and complications associated therewith, atopic eczema (atopic dermatitis), contact dermatitis and further eczematous dermatitis, inflammatory bowel disease (e.g. Crohn's disease and ulcerative colitis), atherosclerosis and amyotrophic lateral sclerosis. Particularly the term refers to COPD, asthma, systemic lupus erythematosis, type I diabetes mellitus and inflammatory bowel disease.

As used herein the term ‘proliferative disease(s)’ refers to conditions such as cancer (e.g. uterine leiomyosarcoma or prostate cancer), myeloproliferative disorders (e.g. polycythemia vera, essential thrombocytosis and myelofibrosis), leukemia (e.g. acute myeloid leukaemia, acute and chronic lymphoblastic leukemia), multiple myeloma, psoriasis, restenosis, scleroderma or fibrosis. In particular the term refers to cancer, leukemia, multiple myeloma and psoriasis.

As used herein, the term ‘cancer’ refers to a malignant or benign growth of cells in skin or in body organs, for example but without limitation, breast, prostate, lung, kidney, pancreas, stomach or bowel. A cancer tends to infiltrate into adjacent tissue and spread (metastasise) to distant organs, for example to bone, liver, lung or the brain. As used herein the term cancer includes both metastatic rumour cell types, such as but not limited to, melanoma, lymphoma, leukaemia, fibrosarcoma, rhabdomyosarcoma, and mastocytoma and types of tissue carcinoma, such as but not limited to, colorectal cancer, prostate cancer, small cell lung cancer and non-small cell lung cancer, breast cancer, pancreatic cancer, bladder cancer, renal cancer, gastric cancer, glioblastoma, primary liver cancer, ovarian cancer, prostate cancer and uterine leiomyosarcoma.

As used herein the term ‘leukemia’ refers to neoplastic diseases of the blood and blood forming organs. Such diseases can cause bone marrow and immune system dysfunction, which renders the host highly susceptible to infection and bleeding. In particular the term leukemia refers to acute myeloid leukaemia (AML), and acute lymphoblastic leukemia (ALL) and chronic lymphoblastic leukaemia (CLL).

As used herein the term ‘transplantation rejection’ refers to the acute or chronic rejection of cells, tissue or solid organ allo- or xenografts of e.g. pancreatic islets, stem cells, bone marrow, skin, muscle, corneal tissue, neuronal tissue, heart, lung, combined heart-lung, kidney, liver, bowel, pancreas, trachea or oesophagus, or graft-versus-host diseases.

As used herein the term ‘diseases involving impairment of cartilage turnover’ includes conditions such as osteoarthritis, psoriatic arthritis, juvenile rheumatoid arthritis, gouty arthritis, septic or infectious arthritis, reactive arthritis, reflex sympathetic dystrophy, algodystrophy, Tietze syndrome or costal chondritis, fibromyalgia, osteochondritis, neurogenic or neuropathic arthritis, arthropathy, endemic forms of arthritis like osteoarthritis deformans endemica, Mseleni disease and Handigodu disease; degeneration resulting from fibromyalgia, systemic lupus erythematosus, scleroderma and ankylosing spondylitis.

As used herein the term ‘congenital cartilage malformation(s)’ includes conditions such as hereditary chondrolysis, chondrodysplasias and pseudochondrodysplasias, in particular, but without limitation, microtia, anotia, metaphyseal chondrodysplasia, and related disorders.

As used herein the term ‘disease(s) associated with hypersecretion of IL6’ includes conditions such as Castleman's disease, multiple myeloma, psoriasis, Kaposi's sarcoma and/or mesangial proliferative glomerulonephritis.

As used herein the term ‘disease(s) associated with hypersecretion of interferons includes conditions such as systemic and cutaneous lupus erythematosis, lupus nephritis, dermatomyositis, Sjogren's syndrome, psoriasis, and/or rheumatoid arthritis.

‘Compound(s) of the invention’, and equivalent expressions, are meant to embrace compounds of the Formula (e) as herein described, which expression includes the pharmaceutically acceptable salts, and the solvates, e.g. hydrates, and the solvates of the pharmaceutically acceptable salts where the context so permits. Similarly, reference to intermediates, whether or not they themselves are claimed, is meant to embrace their salts, and solvates, where the context so permits.

When ranges are referred to herein, for example but without limitation, C₁₋₈ alkyl, the citation of a range should be considered a representation of each member of said range.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particularly useful prodrugs. In some cases it is desirable to prepare double ester type prodrugs such as (acyloxy)alkyl esters or ((alkoxycarbonyl)oxy)alkylesters. Particular such prodrugs are the C₁₋₈ alkyl, C₂₋₈ alkenyl, C₆₋₁₀ optionally substituted aryl, and (C₆₋₁₀ aryl)-(C₁₋₄ alkyl) esters of the compounds of the invention.

As used herein, the term ‘isotopic variant’ refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C ¹⁸F ¹⁵O and ¹³N, and would be useful in Positron Emission Topography (PET) studies for examining substrate receptor occupancy.

All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e. as (+) or (−)-isomers respectively). A chiral compound can exist as either individual enantiomer or as a mixture thereof. A mixture containing equal proportions of the enantiomers is called a ‘racemic mixture’.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base.

Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

The compounds of the invention may possess one or more asymmetric centers; such compounds can therefore be produced as individual (R)— or (S)— stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

It will be appreciated that compounds of the invention may be metabolized to yield biologically active metabolites.

### The Compounds

The present invention is based on the identification that the compounds of the invention are inhibitors of JAK and that they are useful for the treatment of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons. The present invention also provides methods for the production of the compounds of the invention, pharmaceutical compositions comprising a compound of the invention and methods for treating allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons by administering a compound of the invention. In a specific embodiment the compounds of the invention are inhibitors of JAK1 and JAK2.

Accordingly, in a first aspect of the invention, compounds of the invention are disclosed having a Formula I:

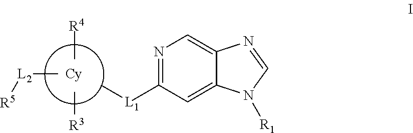

wherein
R¹ is Me, Et, or cyclopropyl, each of which is optionally substituted with one or more halo;

### L₁ is —NR²—; —O—, or —CH₂—;

Cy is phenyl, or 5-9 membered heteroaryl comprising 1 to 4 heteroatoms independently selected from N, O, and S;

### R² is

—C₁₋₄ alkyl optionally substituted with one or more groups independently selected from
   C₃₋₇ cycloalkyl,
   halo,
   CN,
   NR^{15a}R^{15b} (wherein each R^{15a} and R^{15b} is independently selected from C₁₋₄ alkyl),
   4-6 membered heterocycloalkyl containing 1 to 2 heteroatoms independently selected from N, O, and S, and
   C₁₋₄ alkoxy, or
C₃₋₇ cycloalkyl;

### R³ is

—H,
halo,
cyclopropyl,
C₁₋₄ alkyl optionally substituted with one or more halo, or
C₁₋₄ alkoxy optionally substituted with one or more halo;
   R⁴ is H, or halo;

### L₂ is

absent, or is
—W—,
—C₁₋₂ alkylene- (wherein the alkylene is optionally substituted with one CN),
—C₁₋₂ alkylene-W— (wherein the alkylene is optionally substituted with one CN), or
—CH═CH—;
   W is —C(═O)—, —C(═O)O—, —C(═O)NR⁶—, —NR⁶C(═O)—, —NR⁶C(═O)O—, —NR⁶C(═O)NH—, —S—, —SO₂—, —SO₂NR⁶—, —NR⁶SO₂—, —NHSO₂NR⁶—, —O—, or NR⁶;

### R⁵ is:

H,
CN,
C₁₋₆ alkyl optionally substituted with one or more independently selected R⁷ groups,
C₃₋₇ cycloalkyl, optionally substituted with one or more groups independently selected from R¹⁰,
4-7 membered heterocycloalkyl comprising 1 or 2 heteroatoms independently selected from N, O, and S, optionally substituted with one or more groups independently selected from R¹⁰,
4-7 membered heterocycloalkenyl comprising 1 double bond, and comprising 1 or 2 heteroatoms independently selected from N, O, and S, optionally substituted with one or more groups independently selected from R¹⁰,
C₆₋₁₀ aryl optionally substituted with one or more groups independently selected from R¹¹,
5-6 membered heteroaryl comprising 1 to 4 heteroatoms independently selected from N, O, and S, optionally substituted with one or more groups independently selected from R¹¹;
   R⁶ is H, or C₁₋₄ alkyl optionally substituted with CN, C₁₋₂ alkoxy, or C₃₋₆ cycloalkyl;

### R⁷ is

—OH,
CN,
halo,
C₁₋₄ alkoxy,
4-7 membered heterocycloalkyl comprising 1 or 2 heteroatoms independently selected from N, O, and S, optionally substituted with one or more groups independently selected from halo, C₁₋₄ alkyl and oxo,
NR^{8a}R^{8b},
5-6 membered heteroaryl comprising 1 to 4 heteroatoms independently selected from N, O, and S, optionally substituted with one or more groups independently selected from C₁₋₄ alkyl, CN, halo, and C₁₋₄ alkoxy,
phenyl optionally substituted with one or more groups independently selected from C₁₋₄ alkyl, CN, halo, and C₁₋₄ alkoxy,
C₃₋₇ cycloalkyl,
—C(═O)NR^{9a}R^{9b}, or
—OSO₂C₁₋₄ alkyl (which alkyl is optionally substituted with one or more halo), or
—NR^{9c}SO₂C₁₋₄ alkyl (which alkyl is optionally substituted with one or more halo);
   each R^{8a}, and R^{8b} is independently selected from H, and C₁₋₄ alkyl;
   each R^{9a}, R^{9b} and R^{9c} is independently selected from H, and C₁₋₄ alkyl;
   each R¹⁰ is independently selected from oxo or R¹¹;
   each R¹¹ is halo, —CN or -L₃-R¹²;
   L₃ is absent or is —C(═O)—, C(═O)O—, —O—, SO₂—, —C(═O)NR^{13a}, —NR^{13b}C(═O), or NR^{13c};
   each R¹² is
—H,
C₁₋₄ alkyl optionally substituted with one or more groups independently selected from
   halo,
   OH,
   CN,
   C₁₋₄ alkoxy,
   NHC(═O)O—C₁₋₄ alkyl,
   —C(═O)NR^{14a}R^{14b},
   —NR^{14c}R^{14d},
   —C(═O)C₁₋₄ alkyl,
   —C(═O)O—C₁₋₄ alkyl,
   phenyl optionally substituted with halo, C₁₋₄ alkyl, C₁₋₄ alkoxy, and
   4-7 membered heterocycloalkyl comprising 1 or 2 heteroatoms independently selected from N, O, and S, optionally substituted with one or more C₁₋₄ alkyl;
4-7 membered heterocycloalkyl comprising 1 or 2 heteroatoms independently selected from N, O, and S, optionally substituted with one or more groups independently from selected C₁₋₄ alkyl, oxo and CN,
5-6 membered heteroaryl comprising 1 to 4 heteroatoms independently selected from N, O, and S, optionally substituted with one or more independently selected C₁₋₄ alkyl, or
C₃₋₇ cycloalkyl optionally substituted with one or more groups independently selected from OH, halo, C₁₋₄ alkyl, and CN; and
   each R^{13a}, R^{13b}, R^{13c}, R^{14a}, R^{14b}, R^{14c}, and R^{14d}, is independently selected from H, and C₁₋₄ alkyl; provided that R³, R⁴, and -L₂-R⁵ are not all simultaneously H when Cy is C₆ aryl, or 6-membered heteroaryl.

Accordingly, in a further aspect of the invention, compounds of the invention are disclosed having a Formula I:

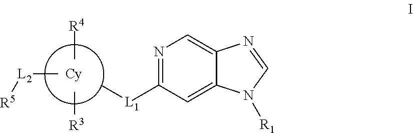

wherein

R¹ is Me, Et, or cyclopropyl, each of which is optionally substituted with one or more halo;

### L₁ is —NR²—; —O—, or —CH₂—;

Cy is phenyl, or 5-9 membered heteroaryl comprising 1 or 2 heteroatoms independently selected from N, O, and S;

### R² is

—C₁₋₂ alkyl optionally substituted with one or more groups independently selected from
   C₃₋₇ cycloalkyl, and
   halo, or
C₃₋₇ cycloalkyl;

### R³ is

—H,
halo,
cyclopropyl,
C₁₋₄ alkyl optionally substituted with one or more halo, or
C₁₋₄ alkoxy optionally substituted with one or more halo;
   R⁴ is H, or halo;

### L₂ is

absent, or is
—W—,
—C₁₋₂ alkylene-, or
—C₁₋₂ alkylene-W—;
   W is —C(═O)—, —C(═O)O—, —C(═O)NR⁶—, —NR⁶C(═O)—, —NR⁶C(═O)O—, —NR⁶C(═O)NH—, —S—, —SO₂—, —SO₂NR⁶—, —NR⁶SO₂—, —O—, or NR⁶;

### R⁵ is:

H,
CN,
C₁₋₆ alkyl optionally substituted with one or more independently selected R⁷ groups,
C₃₋₇ cycloalkyl, optionally substituted with one or more groups independently selected from R¹⁰,
4-7 membered heterocycloalkyl comprising 1 or 2 heteroatoms independently selected from N, O, and S, optionally substituted with one or more groups independently selected from R¹⁰,
4-7 membered heterocycloalkenyl comprising 1 double bond, and comprising 1 or 2 heteroatoms independently selected from N, O, and S, optionally substituted with one or more groups independently selected from R¹⁰,
C₆₋₁₀ aryl optionally substituted with one or more groups independently selected from R¹¹,
5-6 membered heteroaryl comprising 1 or 2 heteroatoms independently selected from N, O, and S, optionally substituted with one or more groups independently selected from R¹¹;
   R⁶ is H, or C₁₋₄ alkyl;

### R⁷ is

—OH,
CN,
halo,
C₁₋₄ alkoxy,
4-7 membered heterocycloalkyl comprising 1 or 2 heteroatoms independently selected from N, O, and S, optionally substituted with one or more groups independently selected from halo, C₁₋₄ alkyl and oxo,

NR^{8a}R^{8b},
5-6 membered heteroaryl comprising 1 or 2 heteroatoms independently selected from N, O, and S, optionally substituted with one or more independently selected C₁₋₄ alkyl, CN, halo, and C₁₋₄ alkoxy,
phenyl optionally substituted with one or more groups independently selected from C₁₋₄ alkyl, CN, halo, and C₁₋₄ alkoxy,
C₃₋₇ cycloalkyl, or
—C(═O)NR^{9a}R^{9b};
   each R^{8a}, and R^{8b} is independently selected from H, and C₁₋₄ alkyl;
   each R^{9a}, and R^{9b} is independently selected from H, and C₁₋₄ alkyl;
   each R¹⁰ is independently selected from oxo or R¹¹;
   each R¹¹ is halo, —CN or -L₃-R¹²;
   L₃ is absent or is —C(═O)—, C(═O)O—, —O—, SO₂—, —C(═O)NR^{13a}, —NR^{13b}C(═O), or NR^{13c};
   each R¹² is
—H,
C₁₋₄ alkyl optionally substituted with one or more groups independently selected from:
   halo,
   OH,
   CN,
   C₁₋₄ alkoxy,
   NHC(═O)O—C₁₋₄ alkyl,
   —C(═O)NR^{14a}R^{14b},
   —NR^{14c}R^{14d},
   —C(═O)C₁₋₄ alkyl,
   —C(═O)O—C₁₋₄ alkyl,
   phenyl optionally substituted with halo, C₁₋₄ alkyl, C₁₋₄ alkoxy, and
   4-7 membered heterocycloalkyl comprising 1 or 2 heteroatoms independently selected from N, O, and S, optionally substituted with one or more C₁₋₄ alkyl;
4-7 membered heterocycloalkyl comprising 1 or 2 heteroatoms independently selected from N, O, and S, optionally substituted with one or more independently selected C₁₋₄ alkyl, oxo and CN,
5-6 membered heteroaryl comprising 1 or 2 heteroatoms independently selected from N, O, and S, optionally substituted with one or more independently selected C₁₋₄ alkyl, or
C₃₋₇ cycloalkyl optionally substituted with one or more independently selected OH, halo, C₁₋₄ alkyl, and CN; and
   each R^{13a}, R^{13b}, R^{13c}, R^{14a}, R^{14b}, R^{14c}, and R^{14d}, is independently selected from H, and C₁₋₄ alkyl;

provided that R³, R⁴, and -L₂-R⁵ are not all simultaneously H when Cy is C₆ aryl, or 6-membered heteroaryl.

In one embodiment, a compound of the invention is according to Formula I, wherein R¹ is Me. In another embodiment, R¹ is Et. In yet another embodiment, R¹ is cyclopropyl.

In one embodiment, a compound of the invention is according to Formula I, wherein R¹ is Me, Et, or cyclopropyl, each of which is substituted with one or more halo. In a particular embodiment, R¹ is Me, or Et, each of which is substituted with one or more halo. In a more particular embodiment, R¹ is Me, or Et, each of which is substituted with one or more F or Cl. In a most particular embodiment, R¹ is CF₃.

In one embodiment, a compound of the invention is according to Formula I, wherein Cy is 5-9-membered heteroaryl comprising 1 to 4 heteroatoms independently selected from N, O, and S. In another embodiment, a compound of the invention is according to Formula I, wherein Cy is 5-9-membered heteroaryl comprising 1 or 2 heteroatoms independently selected from N, O, and S. In a particular embodiment, Cy is pyrazolyl, pyrrolyl, imidazolyl, triazolyl, thiophenyl, thiazolyl, furanyl, pyridyl, pyrazinyl, pyrimidyl, pyridazinyl, benzofuranyl, benzothiophenyl, benzothiazolyl, benzothiadiazolyl, benzoxazolyl, indolyl or indazolyl.

In one embodiment, a compound of the invention is according to Formula I, wherein Cy is 5-9-membered heteroaryl comprising 1 to 4 heteroatoms independently selected from N, O, and S, -L₂ is absent, and R⁵ is H, or C₁₋₆ alkyl (optionally substituted with one or more groups independently selected from CN, and phenyl (optionally substituted with one or more independently selected C₁₋₄ alkoxy)). In another embodiment, a compound of the invention is according to Formula I, wherein Cy is 5-9-membered heteroaryl comprising 1 or 2 heteroatoms independently selected from N, O, and S, -L₂ is absent, and R⁵ is H, or C₁₋₆ alkyl (optionally substituted with one or more groups independently selected from CN, and phenyl (optionally substituted with one or more independently selected C₁₋₄ alkoxy)). In a particular embodiment, Cy is pyrazolyl, pyrrolyl, imidazolyl, triazolyl, thiophenyl, thiazolyl, furanyl, pyridyl, pyrazinyl, pyrimidyl, pyridazinyl, benzofuranyl, benzothiophenyl, indolyl or indazolyl; -L₂ is absent, R⁵ is H, or C₁₋₆ alkyl (optionally substituted with one or more groups independently selected from CN, and phenyl (optionally substituted with one or more independently selected C₁₋₄ alkoxy)). In another particular embodiment, Cy is 5-9-membered heteroaryl comprising 1 to 4 heteroatoms independently selected from N, O, and S; -L₂ is absent, R⁵ is H, Me, Et, n-Pr, or i-Pr each of which is optionally substituted with one or more groups independently selected from CN, and phenyl (optionally substituted with one or more independently selected C₁₋₄ alkoxy)). In yet another particular embodiment, Cy is 5-9-membered heteroaryl comprising 1 or 2 heteroatoms independently selected from N, O, and S; -L₂ is absent, R⁵ is H, Me, Et, n-Pr, or i-Pr each of which is optionally substituted with one or more groups independently selected from CN, and phenyl (optionally substituted with one or more independently selected C₁₋₄ alkoxy)). In a more particular embodiment, Cy is pyrazolyl, pyrrolyl, imidazolyl, triazolyl, thiophenyl, thiazolyl, furanyl, pyridyl, pyrazinyl, pyrimidyl, pyridazinyl, benzofuranyl, benzothiophenyl, indolyl or indazolyl; -L₂ is absent, R⁵ is H, Me, Et, n-Pr, or i-Pr (each of which is optionally substituted with one or more groups independently selected from CN, and phenyl (optionally substituted with one or more independently selected C₁₋₄ alkoxy)). In a further particular embodiment, Cy is benzoxazolyl, benzofaranyl, indazolyl, benzothiazolyl, benzothiadiazolyl; L₂ is absent, R⁵ is H or Me.

In another embodiment, a compound of the invention is according to Formula I, wherein Cy is:

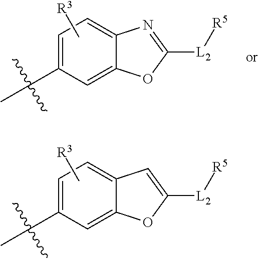

wherein R³ is as described in any of the embodiments above; L₂ is absent, and R⁵ is H, or C₁₋₆ alkyl. In a particular embodiment, L₂ is absent, and R⁵ is H. In another particular embodiment, L₂ is absent, and R⁵ is C₁₋₆ alkyl. In a more particular embodiment, L₂ is absent, and R⁵ is Me, or Et. In a further particular embodiment, L₂ is absent, and R⁵ is Me.

In another embodiment, a compound of the invention is according to Formula I, wherein Cy is:

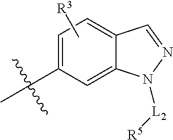

wherein R³ is as described in any of the embodiments above, L₂ is absent, and R⁵ is H, or C₁₋₆ alkyl (optionally substituted with one or more independently CN, or phenyl (optionally substituted with one or more independently selected C₁₋₄ alkoxy)). In a particular embodiment, L₂ is absent, and R⁵ is H. In a further particular embodiment, L₂ is absent, and R⁵ is C₁₋₆ alkyl. In another particular embodiment, L₂ is absent, and R⁵ is Me, Et, n-Pr, each of which is optionally substituted with one or more independently selected CN, or phenyl (optionally substituted with one or more independently selected C₁₋₄ alkoxy). In a more particular embodiment, L₂ is absent, and R⁵ is H, Me, Et, n-Pr, —CH₂—CH₂CN, —CH₂— (phenyl), or —CH₂-(4-OMe-phenyl).

In another embodiment, a compound of the invention is according to Formula I, wherein Cy is 5-6 membered heteroaryl comprising 1 to 4 heteroatoms independently selected from N, O, and S. In yet another embodiment, a compound of the invention is according to Formula I, wherein Cy is 5-6 membered heteroaryl comprising 1 or 2 heteroatoms independently selected from N, O, and S. In a particular embodiment, Cy is pyrazolyl, pyrrolyl, imidazolyl, furanyl, thiophenyl, triazolyl, tetrazolyl, thiazolyl, oxazolyl, thiadiazolyl, or oxadiazolyl. In another particular embodiment, Cy is pyridinyl, pyrazinyl, pyrimidinyl, or pyridazolyl. In a more particular embodiment, Cy is pyridyl.

In another embodiment, a compound of the invention is according to Formula I, wherein Cy is phenyl.

In one embodiment, a compound of the invention is according to Formula I, wherein R⁴ is H, or halo. In a particular embodiment, R⁴ is F, or Cl. In another particular embodiment, R⁴ is H.

In one embodiment, a compound of the invention according to Formula I is according to Formula IIa or IIb:

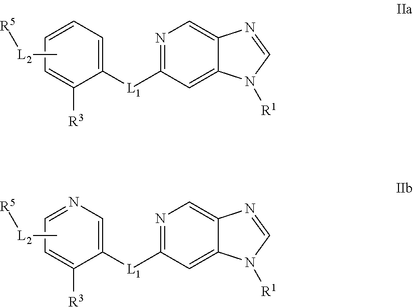

wherein R¹, L₁, R³, L₂, and R⁵ are as described in any of the embodiments above.

In one embodiment, a compound of the invention is according to Formula IIa or IIb, wherein R¹ is Me, Et, or cyclopropyl. In a particular embodiment, R¹ is Me, or Et. In a more particular embodiment, R¹ is Me.

In one embodiment, a compound of the invention according to Formula I is according to Formula IIIa or IIIb:

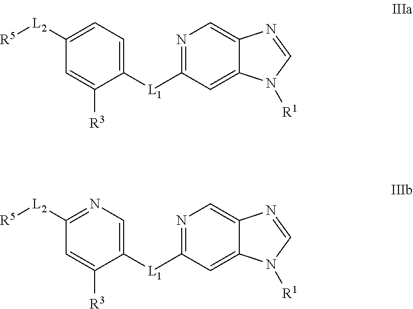

wherein L₁, R³, L₂, and R⁵ are as described in any of the embodiments above.

In one embodiment, a compound of the invention is according to any one of Formulae I-IIIb, wherein L₁ is —CH₂—.

In one embodiment, a compound of the invention is according to any one of Formulae I-IIIb, wherein L₁ is O.

In one embodiment, a compound of the invention is according to any one of Formulae I-IIIb, wherein L₁ is —NR², and R² is as described in any of the embodiments above.

In one embodiment, a compound of the invention is according to any one of Formulae I-IIIb, wherein L₁ is NR², wherein R² is C₁₋₄ alkyl. In a particular embodiment, R² is Me, Et, or iPr. In a more particular embodiment, R² is Me. In another more particular embodiment, R² is Et.

In one embodiment, a compound of the invention is according to any one of Formulae I-IIIb, wherein L₁ is NR², wherein R² is C₁₋₂ alkyl. In a particular embodiment, R² is Me, or Et. In a more particular embodiment, R² is Me. In another more particular embodiment, R² is Et.

In one embodiment, a compound of the invention is according to any one of Formulae I-IIIb, wherein L₁ is NR², wherein R² is C₁₋₄ alkyl substituted with one or more groups independently selected from C₃₋₇ cycloalkyl, halo, CN and C₁₋₄ alkoxy. In a particular embodiment, R² is Me, Et or n-Pr, each of which is substituted with one or more groups independently selected from C₃₋₇ cycloalkyl, halo, CN and C₁₋₄ alkoxy. In a more particular embodiment, R² is Me, Et or n-Pr, each of which is substituted with one group selected from C₃₋₇ cycloalkyl, halo, CN and C₁₋₄ alkoxy. In another particular embodiment, R² is C₁₋₄ alkyl substituted with one or more groups independently selected from cyclopropyl, F, CN, and OMe. In a more particular embodiment, R² is C₁₋₄ alkyl substituted with cyclopropyl. In another more particular embodiment, R² is Me, Et or n-Pr, each of which is substituted with cyclopropyl. In a most particular embodiment, R² is —CH₂-cPr. In another more particular embodiment, R² is Me, Et or iPr, each of which is substituted with one CN, or OMe. In another most particular embodiment, R² is —CH₂—CH₂—CN, or —CH₂—CH₂—OMe.

In one embodiment, the compound of the invention is according to any one of Formulae I-IIIb, wherein L₁ is NR², wherein R² is C₁₋₂ alkyl substituted with one or more groups independently selected from C₃₋₇ cycloalkyl, and halo. In a particular embodiment, R² is Me, or Et, each of which is substituted with one or more groups independently selected from C₃₋₇ cycloalkyl, and halo. In a more particular embodiment, R² is Me, or Et, each of which is substituted with one group selected from C₃₋₇ cycloalkyl, and halo. In another particular embodiment, R² is C₁₋₂ alkyl substituted with one or more groups independently selected from cyclopropyl, and F. In a more particular embodiment, R² is C₁₋₂ alkyl substituted with cyclopropyl. In another more particular embodiment, R² is Me or Et, each of which is substituted with cyclopropyl. In a most particular embodiment, R² is —CH₂-cPr.

In one embodiment, a compound of the invention is according to any one of Formulae I-IIIb,

wherein L₁ is NR², wherein R² is C₁₋₄ alkyl substituted with one 4-6 membered heterocycloalkyl containing 1 to 2 heteroatoms independently selected from N, O, and S. In a particular embodiment, R² is Me, Et or n-Pr, each of which is substituted with one 4-6 membered heterocycloalkyl containing 1 to 2 heteroatoms independently selected from N, O, and S. In another particular embodiment, R² is C₁₋₄ alkyl substituted with one pyrrolyl, piperidinyl, piperazinyl, or morpholinyl. In another more particular embodiment, R² is Me, Et or n-Pr, each of which is substituted with pyrrolyl, piperidinyl, piperazinyl, or morpholinyl. In another most particular embodiment, R² is —CH₂—CH₂-pyrrolyl, —CH₂—CH₂-piperidinyl, CH₂—CH₂-piperazinyl, or —CH₂—CH₂-morpholinyl.

In one embodiment, a compound of the invention is according to any one of Formulae I-IIIb, wherein L₁ is NR², wherein R² is C₁₋₄ alkyl substituted with one —NR^{15a}R^{15b}, wherein each R^{15a} and R^{15b} is independently selected from C₁₋₄ alkyl. In a particular embodiment, R² is Me, Et or n-Pr, each of which is substituted with one NR^{15a}R^{15b}, wherein each R^{15a} and R^{15b} is independently selected from C₁₋₄ alkyl. In another particular embodiment, R² is C₁₋₄ alkyl substituted with one —NR^{15a}R^{15b}, wherein each R^{15a} and R^{15b} is independently selected from Me, Et, or n-Pr. In another more particular embodiment, R² is Me, Et or n-Pr, each of which is substituted with —NMe₂, —NEt₂, or —NMeEt. In another most particular embodiment, R² is —CH₂—CH₂—NMe₂, —CH₂—CH₂—NEt₂, or —CH₂—CH₂—NMeEt.

In one embodiment, a compound of the invention is according to Formulae any one of I-IIIb, wherein L₁ is NR², wherein R² is C₃₋₇ cycloalkyl. In a particular embodiment, R² is cyclopropyl, cyclobutyl, cyclopentyl. In a more particular embodiment, R² is cyclopropyl.

In one embodiment, a compound of the invention is according to any one of Formulae I-IIIb, wherein R³ is H.

In one embodiment, a compound of the invention is according to any one of Formulae I-IIIb, wherein R³ is halo. In a particular embodiment, R³ is F, or Cl. In more particular embodiment, R³ is Cl.

In one embodiment, a compound of the invention is according to any one of Formulae I-IIIb, wherein R³ is cyclopropyl.

In one embodiment, a compound of the invention is according to any one of Formulae I-IIIb, wherein R³ is C₁₋₄ alkyl. In a particular embodiment, R³ is Me, Et, or n-Pr. In more particular embodiment, R³ is Me or Et. In a most particular embodiment, R³ is Et.

In another embodiment, a compound of the invention is according to any one of Formulae I-IIIb, wherein R³ is C₁₋₄ alkyl substituted with halo. In a particular embodiment, R³ is CHF₂, CF₃, —CH₂—CHF₂ or —CH₂—CF₃. In more particular embodiment, R³ is CF₃, or —CH₂—CF₃.

In one embodiment, a compound of the invention is according to any one of Formulae I-IIIb, wherein R³ is C₁₋₄ alkoxy. In a particular embodiment, R³ is —OMe, —OEt, or On-Pr. In more particular embodiment, R³ is —OMe or —OEt.

In another embodiment, a compound of the invention is according to any one of Formulae I-IIIb, wherein R³ is C₁₋₄ alkoxy substituted with halo. In a particular embodiment, R³ is OCHF₂, OCF₃, or —OCH₂—CHF₂. In more particular embodiment, R³ is OCHF₂.

In one embodiment, a compound of the invention is according to any one of Formula I-IIIb, wherein L₂ is absent.

In one embodiment, a compound of the invention is according to any one of Formulae I-IIIb, wherein L₂ is C₁₋₂ alkylene. In a particular embodiment, L₂ is —CH₂— or CH(CH₃)—. In a more particular embodiment, L₂ is —CH₂—.

In another embodiment, a compound of the invention is according to any one of Formulae I-IIIb, wherein L₂ is C₁₋₂ alkylene substituted with one CN. In a particular embodiment, L₂ is CH(CN)—.

In one embodiment, a compound of the invention is according to any one of Formulae I-IIIb, wherein L₂ is W—. In a particular embodiment, —W— is —O—. In another particular embodiment, —W— is —C(═O)—. In another particular embodiment, —W— is —C(═O)O—. In another particular embodiment, —W— is —S—. In another particular embodiment, —W— is —SO₂. In a most particular embodiment, —W— is —C(═O)—.

In one embodiment, a compound of the invention is according to any one of Formulae I-IIIb, wherein L₂ is —C₁₋₂ alkylene-W—. In a particular embodiment, L₂ is —CH₂—W—, —CH₂—CH₂—W— or —CH(CH₃)—W—, and W is as previously described. In another particular embodiment, L₂ is —C₁₋₂ alkylene-O—, —C₁₋₂ alkylene-C(═O)—, —C₁₋₂ alkylene-C(═O)O—, or —C₁₋₂ alkylene-SO₂—. In a more particular embodiment, L₂ is —CH₂—O—, —CH₂—C(═O)—, —CH₂—C(═O)O—, —CH₂—SO₂—, —CH₂—CH₂—O—, —CH₂—CH₂—C(═O)—, —CH₂—CH₂—C(—O)O—, or —CH₂—CH₂—SO₂—.

In one embodiment, a compound of the invention is according to any one of Formulae I-IIIb, wherein L₂ is —C₁₋₂ alkylene-W—. In a particular embodiment, L₂ is —CH₂—W—, —CH₂—CH₂—W— or CH(CH₃)—W—, and W is as previously described. In another particular embodiment, L₂ is —C₁₋₂ alkylene-C(═O)NR⁶—, —C₁₋₂ alkylene-NHSO₂NR⁶—, —C₁₋₂ alkylene-NR⁶C(═O)—, or —C₁₋₂ alkylene-NR⁶SO₂—, wherein R⁶ is as described previously. In another particular embodiment, L₂ is —C₁₋₂ alkylene-C(═O)NR⁶—, —C₁₋₂ alkylene-NHSO₂NR⁶—, —C₁₋₂ alkylene-NR⁶C(═O)—, or —C₁₋₂ alkylene-NR⁶SO₂—, wherein R⁶ is H, —CH₃, or —CH₂—CH₃. In a more particular embodiment, L₂ is —CH₂—C(═O)NR⁶—, —CH₂—NHSO₂NR⁶—, —CH₂—NR⁶C(═O)—, or —CH₂—NR⁶SO₂—, —CH₂—CH₂—C(═O)NR⁶—, —CH₂—CH₂—NHSO₂NR⁶—, —CH₂—CH₂—NR⁶C(═O)—, or —CH₂—CH₂—NR⁶SO₂—, wherein R⁶ is as described previously. In another more particular embodiment, L₂ is —CH₂—C(═O)NR⁶—, —CH₂—NHSO₂NR⁶—, —CH₂—NR⁶C(═O)—, or —CH₂—NR⁶SO₂—, —CH₂—CH₂—C(═O)NR⁶—, —CH₂—CH₂—NHSO₂NR⁶—, —CH₂—CH₂—NR⁶C(═O)—, or —CH₂—CH₂—NR⁶SO₂—, wherein R⁶ is H, —CH₃, or —CH₂—CH₃.

In one embodiment, a compound of the invention is according to any one of Formulae I-IIIb, wherein L₂ is —W—. In a particular embodiment, —W— is —C(═O)NR⁶—, —NR⁶C(═O)—, —SO₂NR⁶—, —NR⁶SO₂—, or NR⁶, wherein R⁶ is H or C₁₋₄ alkyl optionally substituted with CN, C₁₋₂ alkoxy, or C₃₋₆ cycloalkyl. In a particular embodiment, R⁶ is H. In another particular embodiment, R⁶ is Me, Et, n-Pr, i-Pr, —CH₂—CN, —CH₂—OMe, —CH₂-cPr, or —CH₂—CH₂—CN. In a more particular embodiment, R⁶ is Me, —CH₂—CN, or Et.

In one embodiment, a compound of the invention is according to any one of Formulae I-IIIb, wherein L₂ is —W—. In a particular embodiment, —W— is —C(═O)NR⁶—, —NR⁶C(═O)—, —SO₂NR⁶—, —NR⁶SO₂—, or NR⁶, wherein R⁶ is H or C₁ alkyl optionally substituted with CN. In a particular embodiment, R⁶ is H. In another particular embodiment, R⁶ is Me, Et, n-Pr, i-Pr, —CH₂—CN, or —CH₂—CH₂—CN. In a more particular embodiment, R⁶ is Me, —CH₂—CN, or Et.

In one embodiment, a compound of the invention is according to any one of Formulae I-IIIb, wherein L₂ is —W—. In a particular embodiment, —W— is —C(═O)NR⁶—, —NR⁶C(═O)—, —SO₂NR⁶—, —NR⁶SO₂—, or NR⁶, wherein R⁶ is H or C₁₋₄ alkyl. In a particular embodiment, R⁶ is H. In another particular embodiment, R⁶ is Me, Et, n-Pr, or i-Pr. In a more particular embodiment, R⁶ is Me, or Et. In a more particular embodiment, a compound of the invention is according to any one of Formulae I-IIIb, wherein L₂ is —C(═O)NR⁶—, wherein R⁶ is H or C₁₋₄ alkyl. In a particular embodiment, R⁶ is H. In another particular embodiment, R⁶ is Me, Et, n-Pr, or i-Pr. In a more particular embodiment, R⁶ is Me, or Et.

In one embodiment, a compound of the invention according to Formula I is according to any one of Formulae IVa-IVf:

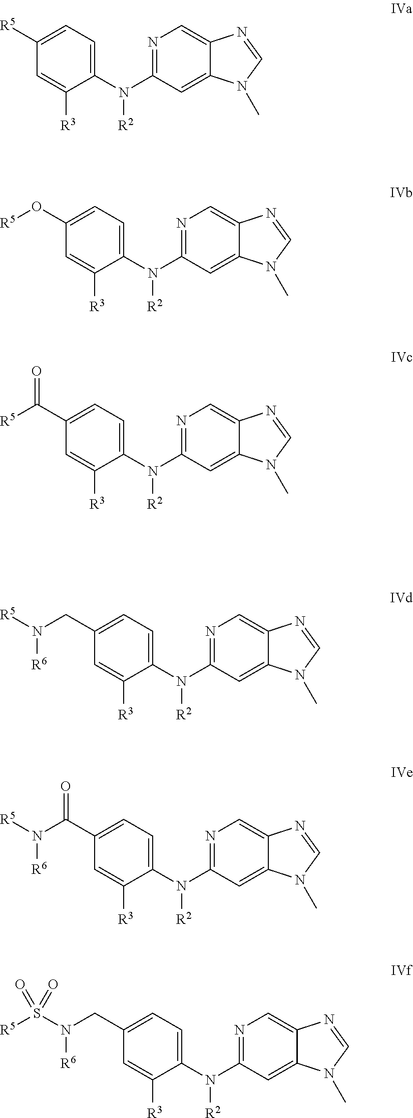

wherein R², R³, R⁶, and R⁵ are as described in any of the embodiments above.

In one embodiment, a compound of the invention according to Formula I is according to any one of Formulae IVg-IVl:

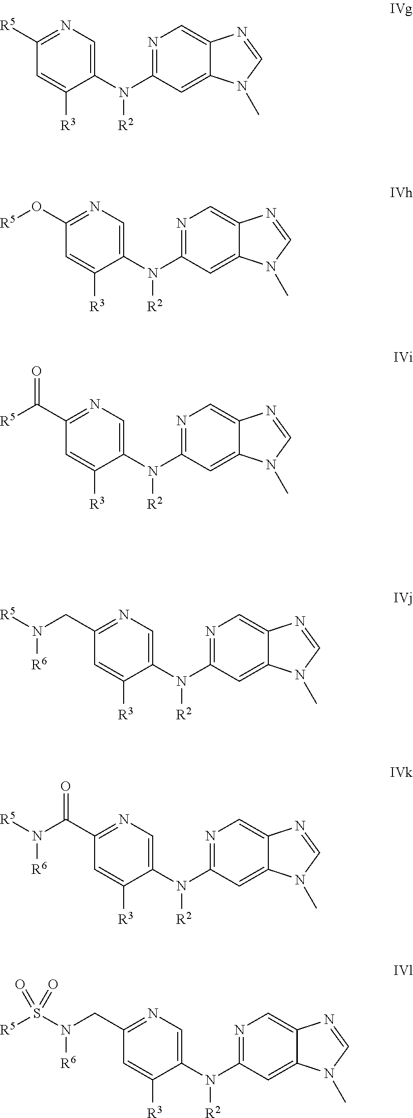

wherein R², R³, R⁶, and R⁵ are as described in any of the embodiments above.

In one embodiment, a compound of the invention is according to any one of Formulae IVa-IVl, wherein R² is C₁₋₂, alkyl. In a particular embodiment, R² is Me, or Et. In a more particular embodiment, R² is Me. In another more particular embodiment, R² is Et. In one embodiment, a compound of the invention is according to any one of Formulae IVa-IVl, wherein R² is C₁₋₂ alkyl substituted with one or more groups independently selected from C₃₋₇ cycloalkyl, and halo. In a particular embodiment, R² is Me, or Et, each of which is substituted with one or more groups independently selected from C₃₋₇ cycloalkyl, and halo. In a more particular embodiment, R² is Me, or Et, each of which is substituted with one group selected from C₃₋₇ cycloalkyl, and halo. In another particular embodiment, R² is C₁₋₂ alkyl substituted with one or more groups independently selected from cyclopropyl, and F. In a more particular embodiment, R² is C₁₋₂ alkyl substituted with cyclopropyl. In another more particular embodiment, R² is Me or Et, each of which is substituted with cyclopropyl. In a most particular embodiment, R² is —CH₂-cPr.

In one embodiment, a compound of the invention is according to any one of Formulae IVa-IVl, wherein R² is C₃₋₇ cycloalkyl. In a particular embodiment, R² is cyclopropyl, cyclobutyl, or cyclopentyl. In a more particular embodiment, R² is cyclopropyl.

In one embodiment, a compound of the invention is according to any one of Formulae IVa-IVl, wherein R³ is H.

In one embodiment, a compound of the invention is according to any one of Formulae IVa-IVl, wherein R³ is halo. In a particular embodiment, R³ is F, or Cl. In more particular embodiment, R³ is Cl.

In one embodiment, a compound of the invention is according to any one of Formulae IVa-IVl, wherein R³ is cyclopropyl.

In one embodiment, a compound of the invention is according to any one of Formulae IVa-IVl, wherein R³ is C₁₋₄ alkyl. In a particular embodiment, R³ is Me, Et, or n-Pr. In more particular embodiment, R³ is Me or Et. In a most particular embodiment, R³ is Et.

In another embodiment, a compound of the invention is according to any one of Formulae IVa-IVl, wherein R³ is C₁₋₄ alkyl substituted with one or more halo. In a particular embodiment, R³ is CHF₂, CF₃, —CH₂—CHF₂ or —CH₂—CF₃. In more particular embodiment, R³ is CF₃, or —CH₂—CF₃.

In one embodiment, a compound of the invention is according to any one of Formulae IVa-IVl, wherein R³ is C₁₋₄ alkoxy. In a particular embodiment, R³ is —OMe, —OEt, or On-Pr. In a more particular embodiment, R³ is —OMe or —OEt.

In another embodiment, a compound of the invention is according to any one of Formulae IVa-IVl, wherein R³ is C₁₋₄ alkoxy substituted with one or more halo. In a particular embodiment, R³ is OCHF₂, OCF₃, or —OCH₂—CHF₂. In more particular embodiment, R³ is OCHF₂.

In one embodiment, a compound of the invention is according to any one of Formulae IVa-IVl, wherein R⁶ is H or C₁₋₄ alkyl. In a particular embodiment, R⁶ is H. In another particular embodiment, R⁶ is Me, Et, n-Pr, or i-Pr. In a more particular embodiment, R⁶ is Me, or Et.

In one embodiment, a compound of the invention is according to any one of Formulae I-IVl, wherein R⁵ is H.

In one embodiment, a compound of the invention is according to Formula IVe, wherein R⁵ is CN.

In one embodiment, a compound of the invention is according to any one of Formulae I-IVl, wherein R⁵ is C₁₋₆ alkyl. In a particular embodiment, R⁵ is Me, Et, n-Pr, i-Pr, or t-Bu. In a particular embodiment, R⁵ is Me, Et, or n-Pr. In a more particular embodiment, R⁵ is Me or Et.

In one embodiment, a compound of the invention is according to any one of Formulae I-IVl, wherein R⁵ is C₁₋₆ alkyl substituted with one or more independently selected R⁷ groups. In another embodiment, R⁵ is C₁₋₆ alkyl substituted with one R⁷ group. In a particular embodiment, R⁵ is Me, Et, or n-Pr, each of which is substituted with one or more independently selected R⁷ groups. In another particular embodiment, R⁵ is Me, Et, or n-Pr, each of which is substituted with one R⁷ group. In a most particular embodiment, R⁵ is Me or Et, each of which is substituted with one R⁷ group.

In one embodiment, a compound of the invention is according to any one of Formulae I-IVl, wherein R⁵ is C₁₋₆ alkyl substituted with one or more independently selected R⁷ groups, and R⁷ is OH, halo, or CN. In a particular embodiment, R⁷ is OH, F, Cl, or CN. In a more particular embodiment, R⁷ is OH.

In one embodiment, a compound of the invention is according to any one of Formulae I-IVl, wherein R⁵ is C₁₋₆ alkyl substituted with one or more independently selected R⁷ groups, and wherein R⁷ is C₁₋₄ alkoxy. In a particular embodiment, R⁷ is —OMe, —OEt, —On-Pr, —Oi-Pr, or —Ot-Bu.

In one embodiment, a compound of the invention is according to any one of Formulae I-IVl, wherein R⁵ is C₁₋₆ alkyl substituted with one or more independently selected R⁷ groups, and R⁷ is 4-7 membered heterocycloalkyl comprising 1 or 2 heteroatoms independently selected from N, O, and S. In a particular embodiment, R⁷ is pyrrolidine, piperidine, piperazine, morpholine or thiomorpholine.

In one embodiment, a compound of the invention is according to any one of Formulae I-IVl, wherein R⁵ is C₁₋₆ alkyl substituted with one or more independently selected R⁷ groups, and R⁷ is 4-7 membered heterocycloalkyl comprising 1 or 2 heteroatoms independently selected from N, O, and S, substituted with one or more groups independently selected from halo, C₁₋₄ alkyl and oxo. In a particular embodiment, R⁷ is 4-7 membered heterocycloalkyl substituted with one group selected from halo, C₁₋₄ alkyl and oxo. In a more particular embodiment, R⁷ is 4-7 membered heterocycloalkyl, substituted with one or more groups independently selected from oxo, Me, Et, i-Pr, t-Bu, F, and Cl. In another more particular embodiment, R⁷ is 4-7 membered heterocycloalkyl, substituted with one group selected from oxo, Me, Et, i-Pr, t-Bu, F, and Cl. In a most particular embodiment, R⁷ is pyrrolidine, piperidine, piperazine, morpholine or thiomorpholine, each of which is substituted with one or more groups independently selected from halo, C₁₋₄ alkyl and oxo. In another most particular embodiment, R⁷ is pyrrolidine, piperidine, piperazine, morpholine or thiomorpholine, each of which is substituted with one group selected from halo, C₁₋₄ alkyl and oxo. In a further most particular embodiment, R⁷ is pyrrolidine, piperidine, piperazine, morpholine or thiomorpholine, each of which is substituted with one or more groups independently selected from oxo, Me, Et, i-Pr, t-Bu, F, and Cl. In an even further most particular embodiment, R⁷ is pyrrolidine, piperidine, piperazine, morpholine or thiomorpholine, each of which is substituted with one group selected from oxo, Me, Et, i-Pr, t-Bu, F, and Cl.

In one embodiment, a compound of the invention is according to any one of Formulae I-IVl, wherein R⁵ is C₁₋₆ alkyl substituted with one or more independently selected R⁷ groups, and R⁷ is NR^{8a}R^{8b}, wherein each R^{8a} and R^{8b} is independently selected from H, and C₁₋₄ alkyl. In a particular embodiment, each R^{8a} and R^{8b} is independently selected from H, Me, and Et. In another particular embodiment, R^{8a} is H and R^{8b} is H, or C₁₋₄ alkyl. In yet another particular embodiment, R^{8a} is H or C₁₋₄ alkyl and R^{8b} is H. In a more particular embodiment, R^{8a} is H and R^{8b} is H, Me, or Et. In another more particular embodiment, R^{8a} is H, Me, or Et and R^{8b} is H. In a most particular embodiment, both R^{8a} and

R^{8b} are H.

In one embodiment, a compound of the invention is according to any one of Formulae I-IVl, wherein R⁵ is C₁₋₆ alkyl substituted with one or more independently selected R⁷ groups, and R⁷ is 5-6 membered heteroaryl comprising 1 to 4 heteroatoms independently selected from N, O, and S. In another embodiment, a compound of the invention is according to any one of Formulae I-IVl, wherein R⁵ is C₁₋₆ alkyl substituted with one or more independently selected R⁷ groups, and R⁷ is 5-6 membered heteroaryl comprising 1 or 2 heteroatoms independently selected from N, O, and S. In a particular embodiment, R⁷ is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl. In a more particular embodiment, R⁷ is imidazolyl, or oxazolyl.

In one embodiment, a compound of the invention is according to any one of Formulae I-IVl, wherein R⁵ is C₁₋₆ alkyl substituted with one or more independently selected R⁷ groups, and R⁷ is 5-6 membered heteroaryl comprising 1 to 4 heteroatoms independently selected from N, O, and S, substituted with one or more independently selected C₁₋₄ alkyl, CN, halo, and C₁₋₄ alkoxy. In one embodiment, a compound of the invention is according to any one of Formulae I-IVl, wherein R⁵ is C₁₋₆ alkyl substituted with one or more independently selected R⁷ groups, and R⁷ is 5-6 membered heteroaryl comprising 1 or 2 heteroatoms independently selected from N, O, and S, substituted with one or more independently selected C₁₋₄ alkyl, CN, halo, and C₁₋₄ alkoxy. In another embodiment, R⁷ is 5-6 membered heteroaryl comprising 1 to 4 heteroatoms independently selected from N, O, and S, substituted with one or two independently selected C₁₋₄ alkyl, CN, halo, and C₁₋₄ alkoxy. In another embodiment, R⁷ is 5-6 membered heteroaryl comprising 1 or 2 heteroatoms independently selected from N, O, and S, substituted with one or two independently selected C₁₋₄ alkyl, CN, halo, and C₁₋₄ alkoxy. In a particular embodiment, R⁷ is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which substituted with one or more independently selected C₁₋₄ alkyl, CN, halo, and C₁₋₄ alkoxy. In another particular embodiment, R⁷ is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which substituted with one or two independently selected C₁₋₄ alkyl, CN, halo, and C₁₋₄ alkoxy. In a more particular embodiment, R⁷ is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which substituted with one or more independently selected Me, Et, CN, F, OMe, and OEt. In another more particular embodiment, R⁷ is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which substituted with one or two independently selected Me, Et, CN, F, OMe, and OEt.

In one embodiment, a compound of the invention is according to any one of Formulae I-IVl, wherein R⁵ is C₁₋₆ alkyl substituted with one or more independently selected R⁷ groups, and R⁷ is phenyl.

In one embodiment, a compound of the invention is according to any one of Formulae I-IVl, wherein R⁵ is C₁₋₆ alkyl substituted with one or more independently selected R⁷ groups, and R⁷ is phenyl substituted with one or more groups independently selected from C₁₋₄ alkyl, CN, halo, and C₁₋₄ alkoxy. In a particular embodiment, R⁷ is phenyl substituted with one C₁₋₄ alkyl, CN, halo, and C₁₋₄ alkoxy. In a more particular embodiment, R⁷ is phenyl substituted with one or more independently selected Me, Et, CN, F, OMe, and OEt. In a most particular embodiment, R⁷ is phenyl substituted with one Me, Et, CN, F, OMe, or OEt.

In one embodiment, a compound of the invention is according to any one of Formulae I-IVl, wherein R⁵ is C₁₋₆ alkyl substituted with one or more independently selected R⁷ groups, and R⁷ is C₃₋₇ cycloalkyl. In a particular embodiment, R⁷ is cyclopropyl.

In one embodiment, a compound of the invention is according to any one of Formulae I-IVl, wherein R⁵ is C₁₋₆ alkyl substituted with one or more independently selected R⁷ groups, and R⁷ is —C(═O)NR^{9a}R^{9b}, wherein each R^{9a} and R^{9b} is independently selected from H, and C₁₋₄ alkyl. In a particular embodiment, each R^{9a} and R^{9b} is independently selected from H, Me, and Et. In another particular embodiment, R^{9a} is H and R^{9b} is H, or C₁₋₄ alkyl. In yet another particular embodiment, R^{9a} is H or C₁₋₄ alkyl and R^{9b} is H. In a more particular embodiment, R^{9a} is H and R^{9b} is H, Me, or Et. In another more particular embodiment, R^{9a} is H, Me, or Et and R^{9b} is H. In a most particular embodiment, both R^{9a} and R^{9b} are H.

In one embodiment, a compound of the invention is according to any one of Formulae I-IVl, wherein R⁵ is C₁₋₆ alkyl substituted with one or more independently selected R⁷ groups, and R⁷ is —OSO₂C₁₋₄ alkyl optionally substituted with one or more halo. In a particular embodiment, R⁷ is −OSO₂—CH₃, —OSO₂—CF₃, —OSO₂—CH₂—CH₃, or —OSO₂—CH(CH₃)₂.

In one embodiment, a compound of the invention is according to any one of Formulae I-IVl, wherein R⁵ is C₁₋₆ alkyl substituted with one or more independently selected R⁷ groups, and R⁷ is —NR^{9c}SO₂C₁₋₄ alkyl, which alkyl is optionally substituted with one or more halo, wherein R^{9c} is selected from H, and C₁₋₄ alkyl. In a particular embodiment, R^{9c} is selected from H, Me, and Et. In another particular embodiment, R⁷ is —NR^{9c}SO₂—CH₃, —NR^{9c}SO₂—CF₃, —NR^{9c}SO₂—CH₂—CH₃, or —NR^{9c}SO₂—CH(CH₃)₂, wherein R^{9c} is selected from H, and C₁₋₄ alkyl. In a more particular embodiment, R^{9c} is selected from H, Me, and Et. In a more particular embodiment, R⁷ is —NHSO₂—CH₃, —NHSO₂—CF₃, —NHSO₂—CH₂—CH₃, or —NHSO₂—CH(CH₃)₂.

In one embodiment, a compound of the invention is according to any one of Formulae I-IVl, wherein R⁵ is C₁₋₆ alkyl substituted with one or more independently selected R⁷ groups, and each R⁷ is selected from —OH, —CN, F, —OMe, —OEt, tetrahydrofuranyl, N-pyrrolidinyl, —N-methylpiperidin-2-yl, —NMe₂, 4-cyano-pyridin-3-yl, 2-pyridinyl, 3-pyridinyl, 2,5-dimethyl-isooxazolyl, phenyl, 4-chlorophenyl, 4-methoxyphenyl, cyclopropyl, and —C(═O)NMe₂. In a particular embodiment, R⁵ is C₁₋₄ alkyl substituted with one R⁷ group, and R⁷ is selected from —OH, —CN, F, —OMe, —OEt, tetrahydrofuranyl, N-pyrrolidinyl, —N-methylpiperidin-2-yl, —NMe₂, 4-cyano-pyridin-3-yl, 2-pyridinyl, 3-pyridinyl, 2,5-dimethyl-isooxazolyl, phenyl, 4-chlorophenyl, 4-methoxyphenyl, cyclopropyl, or —C(═O)NMe₂. In a more particular embodiment R⁵ is Me, Et, or n-Pr, each of which is substituted with one or more independently selected R⁷ groups, and each R⁷ is —OH, —CN, F, —OMe, —OEt, tetrahydrofuranyl, N-pyrrolidinyl, —N-methylpiperidin-2-yl, —NMe₂, 4-cyano-pyridin-3-yl, 2-pyridinyl, 3-pyridinyl, 2,5-dimethyl-isooxazolyl, phenyl, 4-chlorophenyl, 4-methoxyphenyl, cyclopropyl, and —C(═O)NMe₂. In a most particular embodiment R⁵ is Me, Et, or n-Pr, each of which is substituted with one R⁷ group, and

R⁷ is OH, —CN, F, —OMe, —OEt, tetrahydrofuranyl, N-pyrrolidinyl, —N-methylpiperidin-2-yl, —NMe₂, 4-cyano-pyridin-3-yl, 2-pyridinyl, 3-pyridinyl, 2,5-dimethyl-isooxazolyl, phenyl, 4-chlorophenyl, 4-methoxyphenyl, cyclopropyl, or —C(═O)NMe₂.

In one embodiment, a compound of the invention is according to any one of Formulae I-IVl, wherein R⁵ is C₃₋₇ cycloalkyl. In a particular embodiment, R⁵ is cyclopropyl, cyclobutyl, cyclopentyl or cyclohexyl. In a more particular embodiment, R⁵ is cyclopropyl.

In one embodiment, a compound of the invention is according to any one of Formulae I-IVl, wherein R⁵ is C₃₋₇ cycloalkyl substituted with one or more independently selected R¹⁰ groups. In a particular embodiment, R⁵ is C₃₋₇ cycloalkyl substituted with one R¹⁰ group. In a more particular embodiment, R⁵ is cyclopropyl, cyclobutyl, cyclopentyl or cyclohexyl, each of which is substituted with one or more independently selected R¹⁰ groups. In another more particular embodiment, R⁵ is cyclopropyl, cyclobutyl, cyclopentyl or cyclohexyl, each of which is substituted with one R¹⁰ group. In a further particular embodiment, R⁵ is cyclopropyl, substituted with one R⁷ group.

In another embodiment, a compound of the invention is according to any one of Formulae I-IVl, wherein R⁵ is 4-7 membered heterocycloalkyl comprising 1 or 2 heteroatoms independently selected from N, O, and S. In a particular embodiment, R⁵ is pyrrolidine, piperidine, piperazine, morpholine or thiomorpholine.

In another embodiment, a compound of the invention is according to any one of Formulae I-IVl, wherein R⁵ is 4-7 membered heterocycloalkyl comprising 1 or 2 heteroatoms independently selected from N, O, and S, substituted with one or more independently selected R¹⁰ groups. In a particular embodiment, R⁵ is 4-7 membered heterocycloalkyl substituted with one R¹⁰ group. In another embodiment, R⁵ is oxetanyl, azetidinyl, tetrahydrofuranyl, pyrrolidinyl, piperidinyl, piperazinyl, morpholinyl or thiomorpholinyl, each of which is substituted with one or more independently selected R¹⁰ groups. In another embodiment, R⁵ is oxetanyl, azetidinyl, tetrahydrofuranyl, pyrrolidinyl, tetrahydropyranyl, piperidinyl, piperazinyl, morpholinyl or thiomorpholinyl, each of which is substituted with one R¹⁰ group.

In another embodiment, a compound of the invention is according to any one of Formulae I-IVf, wherein R⁵ is 4-7 membered heterocycloalkenyl comprising 1 double bond, and comprising 1 or 2 heteroatoms independently selected from N, O, and S. In a particular embodiment, R⁵ is dihydropyranyl, or tetrahydropyridinyl.

In another embodiment, a compound of the invention is according to any one of Formulae I-IVl, wherein R⁵ is 4-7 membered heterocycloalkenyl comprising 1 double bond, and comprising 1 or 2 heteroatoms independently selected from N, O, and S, substituted with one or more independently selected R¹⁰ groups. In a particular embodiment, R⁵ is 4-7 membered heterocycloalkenyl substituted with one R¹⁰ group. In another embodiment, R⁵ is dihydropyranyl, or tetrahydropyridinyl, each of which is substituted with one or more independently selected R¹⁰ groups. In another embodiment, R⁵ is dihydropyranyl, or tetrahydropyridinyl, each of which is substituted with one R¹⁰ group.

In one embodiment, a compound of the invention is according to any one of Formulae I-IVl, wherein R⁵ is C₆₋₁₀ aryl. In a particular embodiment, R⁵ is phenyl.

In one embodiment, a compound of the invention is according to any one of Formulae I-IVl, wherein R⁵ is C₆₋₁₀ aryl substituted with one or more independently selected R¹¹ groups. In a particular embodiment, R⁵ is C₆₋₁₀ aryl substituted with one R¹¹ group. In a more particular embodiment, R⁵ is phenyl substituted with one or more independently selected R¹¹ groups. In another more particular embodiment, R⁵ is phenyl substituted with one R¹¹ group.

In one embodiment, a compound of the invention is according to any one of Formulae I-IVl, wherein R⁵ is 5-6 membered heteroaryl comprising 1 to 4 heteroatoms independently selected from N, O, and S. In another embodiment, a compound of the invention is according to any one of Formulae I-IVl, wherein R⁵ is 5-6 membered heteroaryl comprising 1 or 2 heteroatoms independently selected from N, O, and S. In a particular embodiment, R⁵ is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl.

In one embodiment, a compound of the invention is according to any one of Formulae I-IVl, wherein R⁵ is 5-6 membered heteroaryl comprising 1 to 4 heteroatoms independently selected from N, O, and S, substituted with one or more independently selected R¹¹ groups. In one embodiment, a compound of the invention is according to any one of Formulae I-IVl, wherein R⁵ is 5-6 membered heteroaryl comprising 1 or 2 heteroatoms independently selected from N, O, and S, substituted with one or more independently selected R¹¹ groups. In a particular embodiment, R⁵ is 5-6 membered heteroaryl comprising 1 to 4 heteroatoms independently selected from N, O, and S, substituted with one R¹¹ group.

In another particular embodiment, R⁵ is 5-6 membered heteroaryl comprising 1 or 2 heteroatoms independently selected from N, O, and S, substituted with one R¹¹ group. In a more particular embodiment, R⁵ is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which is substituted with one or more independently selected R¹¹ groups. In another more particular embodiment, R⁵ is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which is substituted with one R¹¹ group.

In one embodiment, a compound of the invention is according to any one of Formulae I-IVl, R⁵ is C₃₋₇ cycloalkyl, 4-7 membered heterocycloalkyl comprising 1 or 2 heteroatoms independently selected from N, O, and S, or 4-7 membered heterocycloalkenyl comprising 1 double bond, and comprising 1 or 2 heteroatoms independently selected from N, O, and S, each of which is substituted with one R¹⁰ group, and R¹⁰ is oxo.

In another embodiment, a compound of the invention is according to any one of Formulae I-IVl, R⁵ is C₃₋₇ cycloalkyl, 4-7 membered heterocycloalkyl comprising 1 or 2 heteroatoms independently selected from N, O, and S, or 4-7 membered heterocycloalkenyl comprising 1 double bond, and comprising 1 or 2 heteroatoms independently selected from N, O, and S, each of which is substituted with one or more independently selected R¹⁰ group, R¹⁰ is R¹¹, and each R¹¹ is halo or CN. In a particular embodiment, R⁵ is C₃₋₇ cycloalkyl, 4-7 membered heterocycloalkyl comprising 1 or 2 heteroatoms independently selected from N, O, and S, or 4-7 membered heterocycloalkenyl comprising 1 double bond, and comprising 1 or 2 heteroatoms independently selected from N, O, and S, each of which is substituted with one R¹⁰ group, R¹⁰ is R¹¹, and R¹¹ is halo or CN. In a more particular embodiment, R¹¹ is CN, F, or Cl.

In another embodiment, a compound of the invention is according to any one of Formulae I-IVl, R⁵ is C₆₋₁₀ aryl or 5-6 membered heteroaryl comprising 1 or 2 heteroatoms independently selected from N, O, and S, each of which is substituted with one or more independently selected R¹¹, and each R¹¹ is halo or CN. In a particular embodiment, R⁵ is C₆₋₁₀ aryl or 5-6 membered heteroaryl comprising 1 or 2 heteroatoms independently selected from N, O, and S, each of which is substituted with one R¹¹ group, and R¹¹ is halo or CN. In a more particular embodiment, R¹¹ is CN, F, or Cl.

In another embodiment, a compound of the invention is according to any one of Formulae I-IVl, R⁵ is C₃₋₇ cycloalkyl, 4-7 membered heterocycloalkyl comprising 1 or 2 heteroatoms independently selected from N, O, and S, or 4-7 membered heterocycloalkenyl comprising 1 double bond, and comprising 1 or 2 heteroatoms independently selected from N, O, and S, each of which is substituted with one or more independently selected R¹⁰ group, R¹⁰ is R¹¹, R¹¹ is -L₃-R¹², wherein -L₃- and R¹² are as described above. In another embodiment, R⁵ is C₃₋₇ cycloalkyl, 4-7 membered heterocycloalkyl comprising 1 or 2 heteroatoms independently selected from N, O, and S, or 4-7 membered heterocycloalkenyl comprising 1 double bond, and comprising 1 or 2 heteroatoms independently selected from N, O, and S, each of which is substituted with one R¹⁰ group, R¹⁰ is R¹¹, R¹¹ is -L₃-R¹² wherein -L₃- and R¹² are as described above.

In another embodiment, a compound of the invention is according to any one of Formulae I-IVl, wherein R⁵ is according to Formula V:

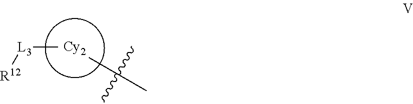

wherein Cy₂ is selected from
C₃₋₇ cycloalkyl,
4-7 membered heterocycloalkyl comprising 1 or 2 heteroatoms independently selected from N, O, and S,
4-7 membered heterocycloalkenyl comprising 1 double bond, and comprising 1 or 2 heteroatoms independently selected from N, O, and S,

C₆₋₁₀ aryl, and
5-6 membered heteroaryl comprising 1 to 4 heteroatoms independently selected from N, O, and S;
   L₃ and R¹² are as described above.

In another embodiment, a compound of the invention is according to any one of Formulae I-IVl, wherein R⁵ is according to Formula V:

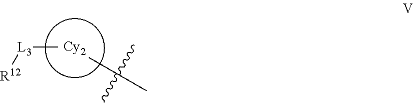

wherein Cy₂ is selected from
C₃₋₇ cycloalkyl,
4-7 membered heterocycloalkyl comprising 1 or 2 heteroatoms independently selected from N, O, and S,
4-7 membered heterocycloalkenyl comprising 1 double bond, and comprising 1 or 2 heteroatoms independently selected from N, O, and S,

C₆₋₁₀ aryl, and
5-6 membered heteroaryl comprising 1 or 2 heteroatoms independently selected from N, O, and S;
   L₃ and R¹² are as described above.

In one embodiment, a compound of the invention is according to any one of Formulae I-IVl, wherein R⁵ is according to Formula V, and Cy₂ is C₃₋₇ cycloalkyl. In a particular embodiment, Cy₂ is cyclopropyl, cyclobutyl, cyclopentyl or cyclohexyl.

In one embodiment, a compound of the invention is according to any one of Formulae I-IVl, wherein R⁵ is according to Formula V, and Cy₂ is 4-7 membered heterocycloalkyl comprising 1 or 2 heteroatoms independently selected from N, O, and S. In a particular embodiment, Cy₂ is oxetanyl, azetidinyl, tetrahydrofuranyl, pyrrolidinyl, tetrahydropyranyl, piperidinyl, piperazinyl, morpholinyl or thiomorpholinyl. In a more particular embodiment, Cy₂ is pyrrolidinyl, piperidinyl, piperazinyl, morpholinyl or thiomorpholinyl. In another more particular embodiment, Cy₂ is azetidinyl.

In one embodiment, a compound of the invention is according to any one of Formulae I-IVl, wherein R⁵ is according to Formula V, and Cy₂ is 4-7 membered heterocycloalkenyl comprising 1 double bond, and comprising 1 or 2 heteroatoms independently selected from N, O, and S. In a particular embodiment, Cy₂ is dihydropyranyl, or tetrahydropyridinyl.

In one embodiment, a compound of the invention is according to any one of Formulae I-IVl, wherein R⁵ is according to Formula V, and Cy₂ is C₆₋₁₀ aryl. In a particular embodiment, Cy₂ is phenyl.

In one embodiment, a compound of the invention is according to Formula I-IVl, wherein R⁵ is according to Formula V, and Cy₂ is 5-6 membered heteroaryl comprising 1 to 4 heteroatoms independently selected from N, O, and S. In one embodiment, a compound of the invention is according to Formula I-IVl, wherein R⁵ is according to Formula V, and Cy₂ is 5-6 membered heteroaryl comprising 1 or 2 heteroatoms independently selected from N, O, and S. In a particular embodiment, Cy₂ is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl.

In one embodiment, a compound of the invention is according to any one of Formulae I-IVl, wherein R⁵ is according to Formula V, and L₃ is absent.

In one embodiment, a compound of the invention is according to any one of Formulae I-IVl, wherein R⁵ is according to Formula V, and L₃ is —C(═O)—.

In one embodiment, a compound of the invention is according to any one of Formulae I-IVl, wherein R⁵ is according to Formula V, and L₃ is —C(═O)O—.

In one embodiment, a compound of the invention is according to any one of Formulae I-IVl, wherein R⁵ is according to Formula V, and L₃ is —O—.

In one embodiment, a compound of the invention is according to any one of Formulae I-IVl, wherein R⁵ is according to Formula V, and L₃ is —SO₂—.

In one embodiment, a compound of the invention is according to any one of Formulae I-IVl, wherein R⁵ is according to Formula V, and L₃ is —C(═O)NR^{13a}, wherein R^{13a} is as defined above. In a more particular embodiment, R^{13a} is H. In a more particular embodiment, R^{13a} is C₁₋₄ alkyl. In a most particular embodiment, R^{13a} is Me, or Et.

In one embodiment, a compound of the invention is according to any one of Formulae I-IVl, wherein R⁵ is according to Formula V, and L₃ is —NR^{13b}C(═O), wherein R^{13b} is as defined above. In a more particular embodiment, R^{13a} is H. In a more particular embodiment, R^{13b} is C₁₋₄ alkyl. In a most particular embodiment, R^{13b} is Me, or Et.

In one embodiment, a compound of the invention is according to any one of Formulae I-IVl, wherein R⁵ is according to Formula V, and L₃ is NR^{13c}, wherein R^{13c} is as defined above. In a more particular embodiment, R^{13c} is H. In a more particular embodiment, R^{13c} is C₁₋₄ alkyl. In a most particular embodiment, R^{13c} is Me, or Et.

In one embodiment, a compound of the invention is according to any one of Formulae I-IVl, wherein R⁵ is according to Formula V, and R¹² is H.

In one embodiment, a compound of the invention is according to any one of Formulae I-IVl, wherein R⁵ is according to Formula V, and R¹² is C₁₋₄ alkyl. In a more particular embodiment, R¹² is Me, Et, Pr, i-Pr or t-Bu.

In one embodiment, a compound of the invention is according to any one of Formulae I-IVl, wherein R⁵ is according to Formula V, and R¹² is C₁₋₄ alkyl substituted with one or more groups independently selected from halo, OH, CN, C₁₋₄ alkoxy, NHC(═O)O—C₁₋₄ alkyl, —C(═O)C₁₋₄ alkyl, and —C(═O)O—C₁₋₄ alkyl. In a another embodiment, R¹² is C₁₋₄ alkyl substituted with one halo, OH, CN, C₁₋₄ alkoxy, NHC(═O)O—C₁₋₄ alkyl, —C(═O)C₁₋₄ alkyl, or —C(═O)O—C₁₋₄ alkyl. In a particular embodiment, R¹² is Me, Et, i-Pr, each of which is substituted with one or more groups independently selected from halo, OH, CN, C₁₋₄ alkoxy, NHC(═O)O—C₁₋₄ alkyl, —C(═O)C₁₋₄ alkyl, and —C(═O)O—C₁₋₄ alkyl. In another particular embodiment, R¹² is Me, Et, i-Pr, each of which is substituted with one halo, OH, CN, C₁₋₄ alkoxy, NHC(═O)O—C₁₋₄ alkyl, —C(═O)C₁₋₄ alkyl, or —C(═O)O—C₁₋₄ alkyl. In a more particular embodiment, R¹² is Me, Et, or i-Pr, each of which is substituted with one or more groups independently selected from F, Cl, OH, CN, OMe, OEt, —NHC(═O)OMe, —NHC(═O)OEt, —NHC(═O)Ot-Bu, —C(═O)Me, —C(═O)Et, —C(═O)t-Bu, —C(═O)OMe, —C(═O)OEt, and —C(═O)Ot-Bu. In another particular embodiment, R¹² is Me, Et, i-Pr, each of which is substituted with F, Cl, OH, CN, OMe, OEt, —NHC(═O)OMe, —NHC(═O)OEt, —NHC(═O)Ot-Bu, —C(═O)Me, —C(═O)Et, —C(═O)t-Bu, —C(═O)OMe, —C(═O)OEt, or —C(═O)Ot-Bu. In a most particular embodiment, R¹² is —CH₂—CN, —CH₂—CH₂—CN, —CH₂—CH₂—OH—C(OH)H—CH₃, —C(OH)H—CF₃, —CHF₂, —CH₂—CF₃, —CH₂—CMe₂-OH, —CMeH—OMe, —CH₂—OH, —CMe₂-OH, —CH₂—OMe, or —CH₂—C(═O)t-Bu.

In one embodiment, a compound of the invention is according to Formula I-IVl, wherein R⁵ is according to Formula V, and R¹² is C₁₋₄ alkyl substituted with one or more independently selected —C(═O)NR^{14a}R^{14b}, wherein each R^{14a} and R^{14b} is independently selected from H and C₁₋₄ alkyl. In a another embodiment, R¹² is C₁₋₄ alkyl substituted with one —C(═O)NR^{14a}R^{14b}, wherein each R^{14a} and R^{14b} is independently selected from H and C₁₋₄ alkyl. In a particular embodiment, R¹² is Me, Et, or i-Pr, each of which is substituted with one or more independently selected —C(═O)NR^{14a}R^{14b}, wherein each R^{14a} and R^{14b} is independently selected from H and C₁₋₄ alkyl. In another particular embodiment, R¹² is Me, Et, or i-Pr, each of which is substituted with one —C(═O)NR^{14a}R^{14b}, wherein each R^{14a} and R^{14b} is independently selected from H or C₁₋₄ alkyl. In a more particular embodiment, R^{14a} is H and R^{14b} is C₁₋₄ alkyl. In a more particular embodiment, R^{14a} is H and R^{14b} is Me or Et. In another more particular embodiment, R^{14a} and R^{14b} are both H. In a most particular embodiment, R¹² is —CH₂—C(═O)NH₂.

In one embodiment, a compound of the invention is according to any one of Formulae I-IVl, wherein R⁵ is according to Formula V, and R¹² is C₁₋₄ alkyl substituted with one or more independently selected —NR^{14c}R^{14d}, wherein each R^{14c} and R^{14d} is independently selected from H and C₁₋₄ alkyl. In another embodiment, R¹² is C₁₋₄ alkyl substituted with one —NR^{14c}R^{14d}, wherein each R^{14c} and R^{14d} is independently selected from H and C₁₋₄ alkyl. In a particular embodiment, R¹² is Me, Et, or i-Pr, each of which is substituted with one or more independently selected —NR^{14c}R^{14d}, wherein each R^{14c} and R^{14d} is independently selected from H and C₁₋₄ alkyl. In another particular embodiment, R¹² is Me, Et, i-Pr substituted with one —NR^{14c}R^{14d} wherein each R^{14c} and R^{14d} is independently selected from H and C₁₋₄ alkyl. In a more particular embodiment, R^{14c} is H and R^{14d} is C₁₋₄ alkyl. In a more particular embodiment, R^{14c} is H and R^{14d} is Me or Et. In another more particular embodiment, R^{14c} and R^{14d} are both H. In a most particular embodiment, R¹² is —CH₂—CH₂—NMe₂.

In one embodiment, a compound of the invention is according to any one of Formulae I-IVl, wherein R⁵ is according to Formula V, and R¹² is C₁₋₄ alkyl substituted with one or more phenyl each of which may independently be optionally substituted with halo, C₁₋₄ alkyl, or Cᵢ alkoxy. In another embodiment, R¹² is C₁₋₄ alkyl substituted with one phenyl optionally substituted with halo, C₁₋₄ alkyl, or C₁₋₄ alkoxy. In a particular embodiment, R¹² is Me, or Et, each of which is substituted with one phenyl.

In another particular embodiment, R¹² is Me, or Et, each of which is substituted with one phenyl substituted with one or more independently selected F, Cl, Me, Et, OMe and OEt. In a most particular embodiment, R¹² is benzyl, —CH₂-4-fluorophenyl, —CH₂-4-chlorophenyl, or —CH₂-4-methylphenyl.

In one embodiment, a compound of the invention is according to any one of Formulae I-IVl, wherein R⁵ is according to Formula V, and R¹² is C₁₋₄ alkyl substituted with one or more 4-7 membered heterocycloalkyl comprising 1 or 2 heteroatoms independently selected from N, O, and S, each of which may independently be optionally substituted with one or more C₁₋₄ alkyl. In a another embodiment, R¹² is C₁₋₄ alkyl substituted with one 4-7 membered heterocycloalkyl comprising 1 or 2 heteroatoms independently selected from N, O, and S, optionally substituted with one or more C₁₋₄ alkyl. In a particular embodiment, R¹² is Me, or Et, each of which is substituted with one 4-7 membered heterocycloalkyl comprising 1 or 2 heteroatoms independently selected from N, O, and S, optionally substituted with one or more C₁₋₄ alkyl. In another particular embodiment, R¹² is Me, or Et, each of which is substituted with one azetidinyl, oxetanyl, tetrahydrofuranyl, pyrrolidinyl, piperidinyl, tetrahydropyranyl, morpholinyl, thiomorpholinyl, or piperazinyl. In another particular embodiment, R¹² is Me, or Et, each of which is substituted with one azetidinyl, oxetanyl, tetrahydrofuranyl, pyrrolidinyl, piperidinyl, tetrahydropyranyl, morpholinyl, thiomorpholinyl, or piperazinyl, each of which is substituted with one or more Me. In a more particular embodiment, R¹² is —CH₂-(1-methyloxetan-3-yl), or —CH₂—CH₂—(N-pyrrolidinyl).

In one embodiment, a compound of the invention is according to any one of Formulae I-IVl, wherein R⁵ is according to Formula V, and R¹² is 4-7 membered heterocycloalkyl comprising 1 or 2 heteroatoms independently selected from N, O, and S, optionally substituted with one or more groups independently selected from C₁₋₄ alkyl, and CN. In another embodiment, R¹² is 4-7 membered heterocycloalkyl comprising 1 or 2 heteroatoms independently selected from N, O, and S, optionally substituted with one C₁₋₄ alkyl, or CN. In a particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is optionally substituted with one or more groups independently selected from C₁₋₄ alkyl, and CN. In another particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is optionally substituted with one C₁₋₄ alkyl, or CN. In a more particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is substituted with one or more groups independently selected from Me, Et and CN. In another particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is substituted with one Me, Et, or CN. In yet another more particular embodiment, R¹² is oxetanyl, azetidinyl, tetrahydrofuranyl, pyrrolidinyl, tetrahydropyranyl, piperidinyl, piperazinyl, morpholinyl or thiomorpholinyl.

In one embodiment, a compound of the invention is according to any one of Formulae I-IVl, wherein R⁵ is according to Formula V, and R¹² is 4-7 membered heterocycloalkyl comprising 1 or 2 heteroatoms independently selected from N, O, and S, optionally substituted with one or more groups independently selected from C₁₋₄ alkyl, oxo, and CN. In another embodiment, R¹² is 4-7 membered heterocycloalkyl comprising 1 or 2 heteroatoms independently selected from N, O, and S, optionally substituted with one C₁₋₄ alkyl, oxo, or CN. In a particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is optionally substituted with one or more groups independently selected from C₁₋₄ alkyl, oxo and CN. In another particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is optionally substituted with one C₁₋₄ alkyl, oxo or CN. In a more particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is substituted with one or more groups independently selected from Me, Et, oxo and CN. In another particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is substituted with one Me, Et, oxo, or CN. In yet another more particular embodiment, R¹² is oxetanyl, azetidinyl, tetrahydrofuranyl, pyrrolidinyl, tetrahydropyranyl, piperidinyl, piperazinyl, morpholinyl or thiomorpholinyl. In a most particular embodiment, R¹² is piperidinyl, morpholinyl, oxetane, piperidinyl substituted with one Me, or CN.

In one embodiment, a compound of the invention is according to any one of Formulae I-IVl, wherein R⁵ is according to Formula V, and R¹² is 5-6 membered heteroaryl comprising 1 to 4 heteroatoms independently selected from N, O, and S, optionally substituted with one or more independently selected C₁₋₄ alkyl. In another embodiment, a compound of the invention is according to any one of Formulae I-IVl, wherein R⁵ is according to Formula V, and R¹² is 5-6 membered heteroaryl comprising 1 or 2 heteroatoms independently selected from N, O, and S, optionally substituted with one or more independently selected C₁₋₄ alkyl. In yet another embodiment, R¹² is 5-6 membered heteroaryl comprising 1 to 4 heteroatoms independently selected from N, O, and S, optionally substituted with one C₁₋₄ alkyl. In another further embodiment, R¹² is 5-6 membered heteroaryl comprising 1 or 2 heteroatoms independently selected from N, O, and S, optionally substituted with one C₁₋₄ alkyl. In a particular embodiment, R¹² is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which is optionally substituted with one or more independently selected C₁₋₄ alkyl. In another particular embodiment, R¹² is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which is optionally substituted with one C₁₋₄ alkyl. In a more particular embodiment, R¹² is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which is substituted with one or more independently selected Me, and Et. In another particular embodiment, R¹² is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which is substituted with one Me, or Et. In yet another more particular embodiment, R¹² is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl. In a most particular embodiment, R¹² is thiadiazolyl.

In one embodiment, a compound of the invention is according to any one of Formulae I-IVl, wherein R⁵ is according to Formula V, and R¹² is C₃₋₇ cycloalkyl optionally substituted with one or more groups independently selected from OH, halo, C₁₋₄ alkyl, and CN. In another embodiment, R¹² is C₃₋₇ cycloalkyl optionally substituted with one OH, halo, C₁₋₄ alkyl, or CN. In a particular embodiment, R¹² is cyclopropyl, cyclobutyl, cyclopentyl, or cyclohexyl, each of which is optionally substituted with one or more groups independently selected from OH, halo, C₁₋₄ alkyl, and CN. In another particular embodiment, R¹² is cyclopropyl, cyclobutyl, cyclopentyl, or cyclohexyl, each of which is optionally substituted with one OH, halo, C₁₋₄ alkyl, or CN. In a more particular embodiment, R¹² is cyclopropyl, cyclobutyl, cyclopentyl, or cyclohexyl, each of which is substituted with one or more independently selected OH, F, Cl, Me, Et, and CN. In another particular embodiment, R¹² is cyclopropyl, cyclobutyl, cyclopentyl, or cyclohexyl, each of which is substituted with one OH, F, Cl, Me, Et, or CN. In yet another more particular embodiment, R¹² is cyclopropyl, cyclobutyl, cyclopentyl, or cyclohexyl.

In one embodiment, a compound of the invention is according to Formula VI:

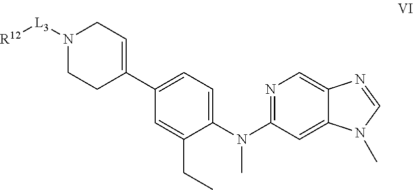

wherein L₃ and R¹² are as described in any of the embodiments above.

In one embodiment, a compound of the invention is according to Formula VII:

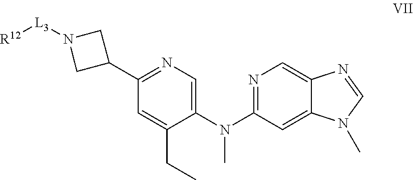

wherein L₃ and R¹² are as described in any of the embodiments above.

In one embodiment, a compound of the invention is according to Formula VI, or Formula VII, wherein L₃ is absent.

In one embodiment, a compound of the invention is according to Formula VI, or Formula VII, wherein L₃ is —C(═O)—.

In one embodiment, a compound of the invention is according to Formula VI, or Formula VII, wherein L₃ is —C(═O)O—.

In one embodiment, a compound of the invention is according to Formula VI, or Formula VII, wherein L₃ is —O—.

In one embodiment, a compound of the invention is according to Formula VI, or Formula VII, wherein L₃ is —SO₂—.

In one embodiment, a compound of the invention is according to Formula VI, or Formula VII, wherein L₃ is —C(═O)NR^{13a}, wherein R^{13a} is as defined above. In a more particular embodiment, R^{13a} is H. In a more particular embodiment, R^{13a} is C₁₋₄ alkyl. In a most particular embodiment, R^{13a} is Me, or Et.

In one embodiment, a compound of the invention is according to Formula VI, or Formula VII, wherein L₃ is —NR^{13b}C(═O), wherein R^{13b} is as defined above. In a more particular embodiment, R^{13a} is H. In a more particular embodiment, R^{13b} is C₁₋₄ alkyl. In a most particular embodiment, R^{13b} is Me, or Et.

In one embodiment, a compound of the invention is according to Formula VI, or Formula VII, wherein L₃ is NR^{13c}, wherein R^{13c} is as defined above. In a more particular embodiment, R^{13c} is H. In a more particular embodiment, R^{13c} is C₁₋₄ alkyl. In a most particular embodiment, R^{13c} is Me, or Et.

In one embodiment, a compound of the invention is according to Formula VI, wherein R¹² is H.

In one embodiment, a compound of the invention is according to Formula VI, or Formula VII, wherein R¹² is C₁₋₄ alkyl. In a more particular embodiment, R¹² is Me, Et, n-Pr, i-Pr or t-Bu.

In one embodiment, a compound of the invention is according to Formula VI, or Formula VII, wherein R¹² is C₁₋₄ alkyl substituted with one or more groups independently selected from halo, OH, CN, C₁₋₄ alkoxy, —NHC(═O)O—C₁₋₄ alkyl, —C(═O)C₁₋₄ alkyl, and —C(═O)O—C₁₋₄ alkyl. In a another embodiment, R¹² is C₁₋₄ alkyl substituted with one halo, OH, CN, C₁₋₄ alkoxy, NHC(═O)O—C₁₋₄ alkyl, —C(═O)C₁₋₄ alkyl, or —C(═O)O—C₁₋₄ alkyl. In a particular embodiment, R¹² is Me, Et, or i-Pr, each of which is substituted with one or more groups independently selected from halo, OH, CN, C₁₋₄ alkoxy, NHC(═O)O—C₁₋₄ alkyl, —C(═O)C₁₋₄ alkyl, and —C(═O)O—C₁₋₄ alkyl. In another particular embodiment, R¹² is Me, Et, or i-Pr, each of which is substituted with one halo, OH, CN, C₁₋₄ alkoxy, —NHC(═O)O—C₁₋₄ alkyl, —C(═O)C₁₋₄ alkyl, or —C(═O)O—C₁₋₄ alkyl. In a more particular embodiment, R¹² is Me, Et, or i-Pr, each of which is substituted with one or more groups independently selected from F, Cl, OH, CN, OMe, OEt, —NHC(═O)OMe, —NHC(═O)OEt, —NHC(═O)Ot-Bu, —C(═O)Me, —C(═O)Et, —C(═O)t-Bu, —C(═O)OMe, —C(═O)OEt, and —C(═O)Ot-Bu. In another particular embodiment, R¹² is Me, Et, or i-Pr, each of which is substituted with one F, Cl, OH, CN, OMe, OEt, —NHC(═O)OMe, —NHC(═O)OEt, —NHC(═O)Ot-Bu, —C(═O)Me, —C(═O)Et, —C(═O)t-Bu, —C(═O)OMe, —C(═O)OEt, or —C(═O)Ot-Bu. In a most particular embodiment, R¹² is —CH₂—CN, —CH₂—CH₂—CN, —CH₂—CH₂—OH—C(OH)H—CH₃, —C(OH)H—CF₃, —CHF₂, —CH₂—CF₃, —CH₂—CMe₂-OH, —CMeH—OMe, —CH₂—OH, —CMe₂-OH, —CH₂—OMe, or —CH₂—C(═O)t-Bu.

In one embodiment, a compound of the invention is according to Formula VI, or Formula VII, wherein R¹² is C₁₋₄ alkyl substituted with one or more independently selected —C(═O)NR^{14a}R^{14b}, wherein each R^{14a} and R^{14b} is independently selected from H and C₁₋₄ alkyl. In a another embodiment, R¹² is C₁₋₄ alkyl substituted with one —C(═O)NR^{14a}R^{14b}, wherein each R^{14a} and R^{14b} is independently selected from H and C₁₋₄ alkyl. In a particular embodiment, R¹² is Me, Et, or i-Pr, each of which is substituted with one or more independently selected —C(═O)NR^{14a}R^{14b}, wherein each R^{14a} and R^{14b} is independently selected from H and C₁₋₄ alkyl. In another particular embodiment, R¹² is Me, Et, or i-Pr, each of which is substituted with one —C(═O)NR^{14a}R^{14b}, wherein each R^{14a} and R^{14b} is independently selected from H and C₁₋₄ alkyl. In a more particular embodiment, R^{14a} is H and R^{14b} is C₁₋₄ alkyl. In a more particular embodiment, R^{14a} is H and R^{14b} is Me or Et. In another more particular embodiment, R^{14a} and R^{14b} are both H. In a most particular embodiment, R¹² is —CH₂—C(═O)NH₂.

In one embodiment, a compound of the invention is according to Formula VI, or Formula VII, wherein R¹² is C₁₋₄ alkyl substituted with one or more independently selected —NR^{14c}R^{14d}, wherein each R^{14c} and R^{14d} is independently selected from H and C₁₋₄ alkyl. In another embodiment, R¹² is C₁₋₄ alkyl substituted with one —NR^{14c}R^{14d}, wherein each R^{14c} and R^{14d} is independently selected from H and C₁₋₄ alkyl. In a particular embodiment, R¹² is Me, Et, or i-Pr, each of which is substituted with one or more independently selected —NR^{14c}R^{14d}, wherein each R^{14c} and R^{14d} is independently selected from H and C₁₋₄ alkyl. In another particular embodiment, R¹² is Me, Et, or i-Pr, each of which is substituted with one —NR^{14c}R^{14d} wherein each R^{14c} and R^{14d} is independently selected from H and C₁₋₄ alkyl. In a more particular embodiment, R^{14c} is H and R^{14d} is C₁₋₄ alkyl. In a more particular embodiment, R^{14c} is H and R^{14d} is Me or Et. In another more particular embodiment, R^{14c} and R^{14d} are both H. In a most particular embodiment, R¹² is —CH₂—CH₂—NMe₂.

In one embodiment, a compound of the invention is according to Formula VI, or Formula VII, wherein R¹² is C₁₋₄ alkyl substituted with one or more phenyl each of which may independently be optionally substituted with halo, C₁₋₄ alkyl, or C₁₋₄ alkoxy. In another embodiment, R¹² is C₁₋₄ alkyl substituted with one phenyl optionally substituted with halo, C₁₋₄ alkyl, or C₁₋₄ alkoxy. In a particular embodiment, R¹² is Me, or Et, each of which is substituted with one phenyl. In another particular embodiment, R¹² is Me, or Et, each of which is substituted with one phenyl substituted with one or more independently selected F, Cl, Me, Et, OMe and OEt. In a most particular embodiment, R¹² is benzyl, CH₂-4-fluorophenyl, —CH₂-4-chlorophenyl, or —CH₂-4-methylphenyl.

In one embodiment, a compound of the invention is according to Formula VI, or Formula VII, wherein R¹² is C₁₋₄ alkyl substituted with one or more 4-7 membered heterocycloalkyl comprising 1 or 2 heteroatoms independently selected from N, O, and S, each of which may independently be optionally substituted with one or more C₁₋₄ alkyl. In a another embodiment, R¹² is C₁₋₄ alkyl substituted with one 4-7 membered heterocycloalkyl comprising 1 or 2 heteroatoms independently selected from N, O, and S, optionally substituted with one or more C₁₋₄ alkyl. In a particular embodiment, R¹² is Me, or Et, each of which is substituted with one 4-7 membered heterocycloalkyl comprising 1 or 2 heteroatoms independently selected from N, O, and S, optionally substituted with one or more C₁₋₄ alkyl. In another particular embodiment, R¹² is Me, or Et, each of which is substituted with one azetidinyl, oxetanyl, tetrahydrofuranyl, pyrrolidinyl, piperidinyl, tetrahydropyranyl, morpholinyl, thiomorpholinyl, or piperazinyl. In another particular embodiment, R¹² is Me, or Et, each of which is substituted with one azetidinyl, oxetanyl, tetrahydrofuranyl, pyrrolidinyl, piperidinyl, tetrahydropyranyl, morpholinyl, thiomorpholinyl, or piperazinyl, each of which is substituted with one or more Me. In a more particular embodiment, R¹² is —CH₂-(1-methyloxetan-3-yl), or —CH₂—CH₂—(N-pyrrolidinyl).

In one embodiment, a compound of the invention is according to Formula VI, or Formula VII, wherein R¹² is 4-7 membered heterocycloalkyl comprising 1 or 2 heteroatoms independently selected from N, O, and S, optionally substituted with one or more groups independently selected from C₁₋₄ alkyl, and CN. In another embodiment, R¹² is 4-7 membered heterocycloalkyl comprising 1 or 2 heteroatoms independently selected from N, O, and S, optionally substituted with one C₁₋₄ alkyl, or CN. In a particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is optionally substituted with one or more groups independently selected from C₁₋₄ alkyl, and CN. In another particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is optionally substituted with one C₁₋₄ alkyl, or CN. In a more particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is substituted with one or more groups independently selected from Me, Et, and CN. In another particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is substituted with one Me, Et, or CN. In yet another more particular embodiment, R¹² is oxetanyl, azetidinyl, tetrahydrofuranyl, pyrrolidinyl, tetrahydropyranyl, piperidinyl, piperazinyl, morpholinyl or thiomorpholinyl.

In one embodiment, a compound of the invention is according to Formula VI, or Formula VII, wherein R¹² is 4-7 membered heterocycloalkyl comprising 1 or 2 heteroatoms independently selected from N, O, and S, optionally substituted with one or more groups independently selected from C₁₋₄ alkyl, oxo, and CN. In another embodiment, R¹² is 4-7 membered heterocycloalkyl comprising 1 or 2 heteroatoms independently selected from N, O, and S, optionally substituted with one C₁₋₄ alkyl, oxo, or CN. In a particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is optionally substituted with one or more independently selected C₁₋₄ alkyl, oxo and CN. In another particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is optionally substituted with one C₁₋₄ alkyl, oxo or CN. In a more particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is substituted with one or more independently selected Me, Et, oxo and CN. In another particular embodiment, R¹² is oxetanyl, azetidinyl, tetrahydrofuranyl, pyrrolidinyl, tetrahydropyranyl, piperidinyl, piperazinyl, morpholinyl or thiomorpholinyl, each of which is substituted with one Me, Et, oxo, or CN. In yet another more particular embodiment, R¹² is oxetanyl, azetidinyl, tetrahydrofuranyl, pyrrolidinyl, tetrahydropyranyl, piperidinyl, piperazinyl, morpholinyl or thiomorpholinyl. In a most particular embodiment, R¹² is piperidinyl, morpholinyl, oxetane, piperidinyl substituted with one Me, or CN.

In one embodiment, a compound of the invention is according to Formula VI, or Formula VII, wherein R¹² is 5-6 membered heteroaryl comprising 1 to 4 heteroatoms independently selected from N, O, and S, optionally substituted with one or more independently selected C₁₋₄ alkyl. In another embodiment, a compound of the invention is according to Formula VI, or Formula VII, wherein R¹² is 5-6 membered heteroaryl comprising 1 or 2 heteroatoms independently selected from N, O, and S, optionally substituted with one or more independently selected C₁₋₄ alkyl. In yet another embodiment, R¹² is 5-6 membered heteroaryl comprising 1 to 4 heteroatoms independently selected from N, O, and S, optionally substituted with one C₁₋₄ alkyl. In another further embodiment, R¹² is 5-6 membered heteroaryl comprising 1 or 2 heteroatoms independently selected from N, O, and S, optionally substituted with one C₁₋₄ alkyl. In a particular embodiment, R¹² is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which is optionally substituted with one or more independently selected C₁₋₄ alkyl. In another particular embodiment, R¹² is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which is optionally substituted with one C₁₋₄ alkyl. In a more particular embodiment, R¹² is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which is substituted with one or more groups independently selected from Me, and Et. In another particular embodiment, R¹² is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl, each of which is substituted with one Me, or Et. In yet another more particular embodiment, R¹² is pyrrolyl, pyrazolyl, furanyl, thiophenyl, imidazolyl, triazolyl, tetrazolyl, oxazolyl, thiazolyl, oxadiazolyl, thiadiazolyl, pyridinyl, or pyrazinyl. In a most particular embodiment, R¹² is thiadiazolyl.

In one embodiment, a compound of the invention is according to Formula VI, or Formula VII, wherein R¹² is C₃₋₇ cycloalkyl optionally substituted with one or more groups independently selected from OH, halo, C₁₋₄ alkyl, and CN. In another embodiment, R¹² is C₃₋₇ cycloalkyl optionally substituted with one OH, halo, C₁₋₄ alkyl, or CN. In a particular embodiment, R¹² is cyclopropyl, cyclobutyl, cyclopentyl, or cyclohexyl, each of which is optionally substituted with one or more groups independently selected from OH, halo, C₁₋₄ alkyl, and CN. In another particular embodiment, R¹² is cyclopropyl, cyclobutyl, cyclopentyl, or cyclohexyl, each of which is optionally substituted with one OH, halo, C₁₋₄ alkyl, or CN. In a more particular embodiment, R¹² is cyclopropyl, cyclobutyl, cyclopentyl, or cyclohexyl, each of which is substituted with one or more groups independently selected from OH, F, Cl, Me, Et, and CN. In another particular embodiment, R¹² is cyclopropyl, cyclobutyl, cyclopentyl, or cyclohexyl, each of which is substituted with one OH, F, Cl, Me, Et, or CN. In yet another more particular embodiment, R¹² is cyclopropyl, cyclobutyl, cyclopentyl, or cyclohexyl.

In one embodiment, a compound of the invention is according to Formula I, wherein the compound is selected from:
N,1-dimethyl-N-(4-(trifluoromethoxy)phenyl)-1H-imidazo[4,5-c]pyridin-6-amine,
N-(4-methoxyphenyl)-N,1-dimethyl-1H-imidazo[4,5-c]pyridin-6-amine,
N-ethyl-N-(1-methyl-1H-imidazo[4,5-c]pyridin-6-yl)benzo[d]oxazol-5-amine,
N-(benzofuran-5-yl)-N-ethyl-1-methyl-1H-imidazo[4,5-c]pyridin-6-amine,
N-ethyl-1-methyl-N-(1-methyl-1H-indazol-5-yl)-1H-imidazo[4,5-c]pyridin-6-amine,
N-ethyl-2-methyl-N-(1-methyl-1H-imidazo[4,5-c]pyridin-6-yl)benzo[d]oxazol-5-amine, ethyl 4-(ethyl(1-methyl-1H-imidazo[4,5-c]pyridin-6-yl)amino)benzate,
N-ethyl-1-methyl-N-(1-methyl-1H-indol-5-yl)-1H-imidazo[4,5-c]pyridin-6-amine,
(3-(ethyl(1-methyl-1H-imidazo[4,5-c]pyridin-6-yl)amino)phenyl)(phenyl)methanone,
N-ethyl-2-methyl-N-(1-methyl-1H-imidazo[4,5-c]pyridin-6-yl)benzo[d]thiazol-5-amine,
N-ethyl-1-methyl-N-(3-phenoxyphenyl)-1H-imidazo[4,5-c]pyridin-6-amine,
(3-(ethyl(1-methyl-1H-imidazo[4,5-c]pyridin-6-yl)amino)phenyl)(phenyl)MeOH, (4-(ethyl(1-methyl-1H-imidazo[4,5-c]pyridin-6-yl)amino)phenyl)MeOH,
N-(4-(benzyloxy)phenyl)-N,1-dimethyl-1H-imidazo[4,5-c]pyridin-6-amine,
N,1-dimethyl-N-o-tolyl-1H-imidazo[4,5-c]pyridin-6-amine,
N-(2-ethylphenyl)-N,1-dimethyl-1H-imidazo[4,5-c]pyridin-6-amine,
4-(methyl(1-methyl-1H-imidazo[4,5-c]pyridin-6-yl)amino)phenol,
N,1-dimethyl-N-(4-((tetrahydrofuran-2-yl)methoxy)phenyl)-1H-imidazo[4,5-c]pyridin-6-amine,
2-(4-(methyl(1-methyl-1H-imidazo[4,5-c]pyridin-6-yl)amino)phenoxy)ethanol,
N-(4-((3,5-dimethylisoxazol-4-yl)methoxy)phenyl)-N,1-dimethyl-1H-imidazo[4,5-c]pyridin-6-amine,
N-(4-(isopentyloxy)phenyl)-N,1-dimethyl-1H-imidazo[4,5-c]pyridin-6-amine,
N,1-dimethyl-N-(4-propoxyphenyl)-1H-imidazo[4,5-c]pyridin-6-amine,
N-(4-(2-ethoxyethoxy)phenyl)-N,1-dimethyl-1H-imidazo[4,5-c]pyridin-6-amine,
N-(4-(cyclopropylmethoxy)phenyl)-N,1-dimethyl-1H-imidazo[4,5-c]pyridin-6-amine,
N-methyl-N-(1-methyl-1H-imidazo[4,5-c]pyridin-6-yl)benzo[c][1,2,5]thiadiazol-5-amine,
2-(4-(methyl(1-methyl-1H-imidazo[4,5-c]pyridin-6-yl)amino)phenyl)acetonitrile, methyl 3-methyl-4-(methyl(1-methyl-1H-imidazo[4,5-c]pyridin-6-yl)amino)benzate,
N-(2-ethyl-4-fluorophenyl)-N,1-dimethyl-1H-imidazo[4,5-c]pyridin-6-amine,
(3-methyl-4-(methyl(1-methyl-1H-imidazo[4,5-c]pyridin-6-yl)amino)phenyl)MeOH,
N,3-dimethyl-4-(methyl(1-methyl-1H-imidazo[4,5-c]pyridin-6-yl)amino)benzamide,
3-methyl-4-(methyl(1-methyl-1H-imidazo[4,5-c]pyridin-6-yl)amino)benzonitrile,
3-methyl-4-(methyl(1-methyl-1H-imidazo[4,5-c]pyridin-6-yl)amino)benzamide,
N-(4-(4-methoxybenzyloxy)-2-methylphenyl)-N,1-dimethyl-1H-imidazo[4,5-c]pyridin-6-amine,
N-ethyl-N-(2-ethylphenyl)-1-methyl-1H-imidazo[4,5-c]pyridin-6-amine,
N-(cyclopropylmethyl)-N-(2-ethylphenyl)-1-methyl-1H-imidazo[4,5-c]pyridin-6-amine,
N,1-dimethyl-N-(2-(trifluoromethyl)phenyl)-1H-imidazo[4,5-c]pyridin-6-amine,
N-cyclopropyl-N-(2-ethylphenyl)-1-methyl-1H-imidazo[4,5-c]pyridin-6-amine,
3-methyl-4-(methyl(1-methyl-1H-imidazo[4,5-c]pyridin-6-yl)amino)phenol,
N-(2-(difluoromethoxy)phenyl)-N,1-dimethyl-1H-imidazo[4,5-c]pyridin-6-amine,
3-methyl-4-(methyl(1-methyl-1H-imidazo[4,5-c]pyridin-6-yl)amino)benzic acid,
2-(methyl(1-methyl-1H-imidazo[4,5-c]pyridin-6-yl)amino)benzonitrile,
N-(2-methoxyphenyl)-N,1-dimethyl-1H-imidazo[4,5-c]pyridin-6-amine,
N-(5-(4-methoxybenzyloxy)-2-methylphenyl)-N,1-dimethyl-1H-imidazo[4,5-c]pyridin-6-amine,
N-(3-(4-methoxybenzyloxy)-2-methylphenyl)-N,1-dimethyl-1H-imidazo[4,5-c]pyridin-6-amine,
N-(4-methoxy-2-methylphenyl)-N,1-dimethyl-1H-imidazo[4,5-c]pyridin-6-amine,
2-methyl-3-(methyl(1-methyl-1H-imidazo[4,5-c]pyridin-6-yl)amino)phenol,
2-(3-methyl-4-(methyl(1-methyl-1H-imidazo[4,5-c]pyridin-6-yl)amino)phenoxy)acetonitrile,
4-(3-methyl-4-(methyl(1-methyl-1H-imidazo[4,5-c]pyridin-6-yl)amino)phenoxy)butanenitrile,
N,1-dimethyl-N-(2-(trifluoromethoxy)phenyl)-1H-imidazo[4,5-c]pyridin-6-amine,
N-(2-methoxyethyl)-3-methyl-4-(methyl(1-methyl-1H-imidazo[4,5-c]pyridin-6-yl)amino)benzamide,
N,N-dimethyl-2-(3-methyl-4-(methyl(1-methyl-1H-imidazo[4,5-c]pyridin-6-yl)amino)phenoxy)acetamide,
N,1-dimethyl-N-(2-methyl-4-(2-(pyrrolidin-1-yl)ethoxy)phenyl)-1H-imidazo[4,5-c]pyridin-6-amine,
N-(4-(benzyloxy)-2-methylphenyl)-N,1-dimethyl-1H-imidazo[4,5-c]pyridin-6-amine,
N-(4-(4-chlorobenzyloxy)-2-methylphenyl)-N,1-dimethyl-1H-imidazo[4,5-c]pyridin-6-amine,
N,1-dimethyl-N-(2-methyl-4-(pyridin-3-ylmethoxy)phenyl)-1H-imidazo[4,5-c]pyridin-6-amine,
N-(5-(benzyloxy)-2-methylphenyl)-N,1-dimethyl-1H-imidazo[4,5-c]pyridin-6-amine,
2-(4-methyl-3-(methyl(1-methyl-1H-imidazo[4,5-c]pyridin-6-yl)amino)phenoxy)acetonitrile,
N,1-dimethyl-N-(2-methyl-5-(2-(pyrrolidin-1-yl)ethoxy)phenyl)-1H-imidazo[4,5-c]pyridin-6-amine,
N,N-dimethyl-2-(4-methyl-3-(methyl(1-methyl-1H-imidazo[4,5-c]pyridin-6-yl)amino)phenoxy)acetamide,
4-(4-methyl-3-(methyl(1-methyl-1H-imidazo[4,5-c]pyridin-6-yl)amino)phenoxy)butanenitrile,
N,1-dimethyl-N-(2-methyl-5-(pyridin-3-ylmethoxy)phenyl)-1H-imidazo[4,5-c]pyridin-6-amine,
N-(5-(4-chlorobenzyloxy)-2-methylphenyl)-N,1-dimethyl-1H-imidazo[4,5-c]pyridin-6-amine,
N-(5-isobutoxy-2-methylphenyl)-N,1-dimethyl-1H-imidazo[4,5-c]pyridin-6-amine,
2-(2-methyl-3-(methyl(1-methyl-1H-imidazo[4,5-c]pyridin-6-yl)amino)phenoxy)acetonitrile,
N,N-dimethyl-2-(2-methyl-3-(methyl(1-methyl-1H-imidazo[4,5-c]pyridin-6-yl)amino)phenoxy)acetamide,
4-(2-methyl-3-(methyl(1-methyl-1H-imidazo[4,5-c]pyridin-6-yl)amino)phenoxy)butanenitrile,
N-(2-fluorophenyl)-N,1-dimethyl-1H-imidazo[4,5-c]pyridin-6-amine,
N-(2-chlorophenyl)-N,1-dimethyl-1H-imidazo[4,5-c]pyridin-6-amine,
N,1-dimethyl-N-(2-methyl-3-(pyridin-3-ylmethoxy)phenyl)-1H-imidazo[4,5-c]pyridin-6-amine,
N-(3-isobutoxy-2-methylphenyl)-N,1-dimethyl-1H-imidazo[4,5-c]pyridin-6-amine,
N,1-dimethyl-N-(2-methyl-4-(1H-pyrazol-4-yl)phenyl)-1H-imidazo[4,5-c]pyridin-6-amine,
N,1-dimethyl-N-(2-methyl-4-(pyrimidin-5-yl)phenyl)-1H-imidazo[4,5-c]pyridin-6-amine,
N,1-dimethyl-N-(2-methyl-4-(1-methyl-1H-pyrazol-4-yl)phenyl)-1H-imidazo[4,5-c]pyridin-6-amine,
N-(4-(4-methoxybenzyl)-2-methylphenyl)-N,1-dimethyl-1H-imidazo[4,5-c]pyridin-6-amine,
N-(3-(benzyloxy)-2-methylphenyl)-N,1-dimethyl-1H-imidazo[4,5-c]pyridin-6-amine,
N-(3-(4-chlorobenzyloxy)-2-methylphenyl)-N,1-dimethyl-1H-imidazo[4,5-c]pyridin-6-amine,
N,1-dimethyl-N-(2-methyl-3-(2-(pyrrolidin-1-yl)ethoxy)phenyl)-1H-imidazo[4,5-c]pyridin-6-amine,
N-(4-ethylpyridin-3-yl)-N,1-dimethyl-1H-imidazo[4,5-c]pyridin-6-amine,
N-(2-ethylpyridin-3-yl)-N,1-dimethyl-1H-imidazo[4,5-c]pyridin-6-amine,
methyl 3-ethyl-4-(methyl(1-methyl-1H-imidazo[4,5-c]pyridin-6-yl)amino)benzate,
(3-ethyl-4-(methyl(1-methyl-1H-imidazo[4,5-c]pyridin-6-yl)amino)phenyl)MeOH,
3-ethyl-4-(methyl(1-methyl-1H-imidazo[4,5-c]pyridin-6-yl)amino)benzonitrile,
4-ethyl-3-(methyl(1-methyl-1H-imidazo[4,5-c]pyridin-6-yl)amino)benzonitrile,
N-(2-ethyl-5-fluorophenyl)-N,1-dimethyl-1H-imidazo[4,5-c]pyridin-6-amine,
3-ethyl-N-methyl-4-(methyl(1-methyl-1H-imidazo[4,5-c]pyridin-6-yl)amino)benzamide,
N-(2-cyanoethyl)-3-ethyl-4-(methyl(1-methyl-1H-imidazo[4,5-c]pyridin-6-yl)amino)benzamide,
4-ethyl-N,N-dimethyl-3-(methyl(1-methyl-1H-imidazo[4,5-c]pyridin-6-yl)amino)benzamide,
5-(3-methyl-4-(methyl(1-methyl-1H-imidazo[4,5-c]pyridin-6-yl)amino)phenoxy)picolinonitrile,
5-((3-methyl-4-(methyl(1-methyl-1H-imidazo[4,5-c]pyridin-6-yl)amino)phenoxy)methyl)picolinonitrile,
5-(2-methyl-3-(methyl(1-methyl-1H-imidazo[4,5-c]pyridin-6-yl)amino)phenoxy)picolinonitrile,
5-(4-methyl-3-(methyl(1-methyl-1H-imidazo[4,5-c]pyridin-6-yl)amino)phenoxy)picolinonitrile,
5-((4-methyl-3-(methyl(1-methyl-1H-imidazo[4,5-c]pyridin-6-yl)amino)phenoxy)methyl)picolinonitrile,
N-(2-ethyl-4-(1-methyl-1H-pyrazol-4-yl)phenyl)-N,1-dimethyl-1H-imidazo[4,5-c]pyridin-6-amine,
N-(2-ethyl-4-(pyridin-3-yl)phenyl)-N,1-dimethyl-1H-imidazo[4,5-c]pyridin-6-amine,
N-(2-ethyl-4-(pyrimidin-5-yl)phenyl)-N,1-dimethyl-1H-imidazo[4,5-c]pyridin-6-amine,
methyl 2-ethyl-3-(methyl(1-methyl-1H-imidazo[4,5-c]pyridin-6-yl)amino)benzate,
5-((2-methyl-3-(methyl(1-methyl-1H-imidazo[4,5-c]pyridin-6-yl)amino)phenoxy)methyl)picolinonitrile,
N-benzyl-3-ethyl-4-(methyl(1-methyl-1H-imidazo[4,5-c]pyridin-6-yl)amino)benzamide,
3-ethyl-4-(methyl(1-methyl-1H-imidazo[4,5-c]pyridin-6-yl)amino)-N-phenethylbenzamide,
(3-ethyl-4-(methyl(1-methyl-1H-imidazo[4,5-c]pyridin-6-yl)amino)phenyl)(4-methylpiperazin-1-yl)methanone,
1-(3-ethyl-4-(methyl(1-methyl-1H-imidazo[4,5-c]pyridin-6-yl)amino)phenyl)ethanol,
N,3-diethyl-4-(methyl(1-methyl-1H-imidazo[4,5-c]pyridin-6-yl)amino)benzamide,
2-(3-ethyl-4-(methyl(1-methyl-1H-imidazo[4,5-c]pyridin-6-yl)amino)phenyl)propan-2-ol,
(1,1-Dioxothiomorpholin-4-yl)-{3-ethyl-4-[methyl-(1-methyl-1H-imidazo[4,5-c]pyridin-6-yl)-amino]-phenyl}-methanone,
N-(2-ethyl-4-(methoxymethyl)phenyl)-N,1-dimethyl-1H-imidazo[4,5-c]pyridin-6-amine,
(3-(dimethylamino)pyrrolidin-1-yl)(3-ethyl-4-(methyl(1-methyl-1H-imidazo[4,5-c]pyridin-6-yl)amino)phenyl)methanone,
3-ethyl-4-(methyl(1-methyl-1H-imidazo[4,5-c]pyridin-6-yl)amino)phenol,
4-ethyl-3-(methyl(1-methyl-1H-imidazo[4,5-c]pyridin-6-yl)amino)benzamide,
(4-ethyl-3-(methyl(1-methyl-1H-imidazo[4,5-c]pyridin-6-yl)amino)phenyl)MeOH,
N-(2-ethyl-4-(1-(methylsulfonyl)pyrrolidin-3-yloxy)phenyl)-N,1-dimethyl-1H-imidazo[4,5-c]pyridin-6-amine,
N-(4-((1H-imidazol-1-yl)methyl)-2-ethylphenyl)-N,1-dimethyl-1H-imidazo[4,5-c]pyridin-6-amine,
(3-ethyl-4-(methyl(1-methyl-1H-imidazo[4,5-c]pyridin-6-yl)amino)phenyl)(4-methoxypiperidin-1-yl)methanone,
(3,3-dimethylazetidin-1-yl)(3-ethyl-4-(methyl(1-methyl-1H-imidazo[4,5-c]pyridin-6-yl)amino)phenyl)methanone,
5-(3-ethyl-4-(methyl(1-methyl-1H-imidazo[4,5-c]pyridin-6-yl)amino)phenyl)picolinonitrile,
N-(5-(3-ethyl-4-(methyl(1-methyl-1H-imidazo[4,5-c]pyridin-6-yl)amino)phenyl)pyrimidin-2-yl)acetamide,
4-ethyl-N-(2-methoxyethyl)-3-(methyl(1-methyl-1H-imidazo[4,5-c]pyridin-6-yl)amino)benzamide,
(3-ethyl-4-(methyl(1-methyl-1H-imidazo[4,5-c]pyridin-6-yl)amino)phenyl)(morpholino)methanone,
3-ethyl-N,N-dimethyl-4-(methyl(1-methyl-1H-imidazo[4,5-c]pyridin-6-yl)amino)benzamide,
N-(2-(dimethylamino)ethyl)-3-ethyl-4-(methyl(1-methyl-1H-imidazo[4,5-c]pyridin-6-yl)amino)benzamide,
N-(cyanomethyl)-3-ethyl-4-(methyl(1-methyl-1H-imidazo[4,5-c]pyridin-6-yl)amino)benzamide,
N-(2-ethyl-4-(2-methoxypyrimidin-5-yl)phenyl)-N,1-dimethyl-1H-imidazo[4,5-c]pyridin-6-amine,
N-(2-ethyl-5-(methoxymethyl)phenyl)-N,1-dimethyl-1H-imidazo[4,5-c]pyridin-6-amine,
N-tert-butyl-4-ethyl-3-(methyl(1-methyl-1H-imidazo[4,5-c]pyridin-6-yl)amino)benzamide,
4-ethyl-3-(methyl(1-methyl-1H-imidazo[4,5-c]pyridin-6-yl)amino)-N-(pyridin-3-yl)benzamide,
4-ethyl-3-(methyl(1-methyl-1H-imidazo[4,5-c]pyridin-6-yl)amino)-N-(pyridin-2-ylmethyl)benzamide,
N-(2-ethyl-4-(thiophen-2-yl)phenyl)-N,1-dimethyl-1H-imidazo[4,5-c]pyridin-6-amine,
N-(2-ethyl-4-(thiophen-3-yl)phenyl)-N,1-dimethyl-1H-imidazo[4,5-c]pyridin-6-amine,
4-ethyl-N-methyl-3-(methyl(1-methyl-1H-imidazo[4,5-c]pyridin-6-yl)amino)benzamide,
N-(4-(2-(dimethylamino)pyrimidin-5-yl)-2-ethylphenyl)-N,1-dimethyl-1H-imidazo[4,5-c]pyridin-6-amine,
N-(2-ethyl-4-(6-methoxypyridin-3-yl)phenyl)-N,1-dimethyl-1H-imidazo[4,5-c]pyridin-6-amine,
N-(2-ethyl-4-(6-methylpyridin-3-yl)phenyl)-N,1-dimethyl-1H-imidazo[4,5-c]pyridin-6-amine,
5-(3-ethyl-4-(methyl(1-methyl-1H-imidazo[4,5-c]pyridin-6-yl)amino)phenyl)pyridin-2-ol,
N-(2-ethyl-4-(5-fluoropyridin-3-yl)phenyl)-N,1-dimethyl-1H-imidazo[4,5-c]pyridin-6-amine,
5-(3-ethyl-4-(methyl(1-methyl-1H-imidazo[4,5-c]pyridin-6-yl)amino)phenyl)pyrimidin-2-ol,
N-(2-ethyl-4-((methylamino)methyl)phenyl)-N,1-dimethyl-1H-imidazo[4,5-c]pyridin-6-amine,
N-(2-ethyl-4-((4-(methylsulfonyl)piperazin-1-yl)methyl)phenyl)-N,1-dimethyl-1H-imidazo[4,5-c]pyridin-6-amine,
3-ethyl-4-(methyl(1-methyl-1H-imidazo[4,5-c]pyridin-6-yl)amino)benzamide,
N-(2-ethyl-4-(1,3,5-trimethyl-1H-pyrazol-4-yl)phenyl)-N,1-dimethyl-1H-imidazo[4,5-c]pyridin-6-amine,
N-(4-(1-benzyl-1H-pyrazol-4-yl)-2-ethylphenyl)-N,1-dimethyl-1H-imidazo[4,5-c]pyridin-6-amine,
N-(2-ethyl-4-(2-isopropylpyrimidin-5-yl)phenyl)-N,1-dimethyl-1H-imidazo[4,5-c]pyridin-6-amine,
N-(2-ethyl-4-((4-methylpiperazin-1-yl)methyl)phenyl)-N,1-dimethyl-1H-imidazo[4,5-c]pyridin-6-amine,
N-(2-ethyl-4-(morpholinomethyl)phenyl)-N,1-dimethyl-1H-imidazo[4,5-c]pyridin-6-amine,
[4-(1,1-Dioxothiomorpholin-4-ylmethyl)-2-ethyl-phenyl]-methyl-(1-methyl-1H-imidazo[4,5-c]pyridin-6-yl)-amine,
N-(2-ethyl-4-(pyrazin-2-yl)phenyl)-N,1-dimethyl-1H-imidazo[4,5-c]pyridin-6-amine,
N-(2-ethyl-4-(1-methyl-1H-imidazol-4-yl)phenyl)-N,1-dimethyl-1H-imidazo[4,5-c]pyridin-6-amine,
{4-[(1,1-Dioxo-hexahydrothiopyran-4-ylamino)-methyl]-2-ethyl-phenyl}-methyl-(1-methyl-1H-imidazo[4,5-c]pyridin-6-yl)-amine,
N-(2-ethyl-4-(5-methyl-1,2,4-oxadiazol-3-yl)phenyl)-N,1-dimethyl-1H-imidazo[4,5-c]pyridin-6-amine,
2-(3-(3-ethyl-4-(methyl(1-methyl-1H-imidazo[4,5-c]pyridin-6-yl)amino)phenyl)-1,2,4-oxadiazol-5-yl)acetonitrile,
2-(3-ethyl-4-(methyl(1-methyl-1H-imidazo[4,5-c]pyridin-6-yl)amino)benzylamino)acetonitrile,
N-(2-ethyl-4-(thiazol-4-yl)phenyl)-N,1-dimethyl-1H-imidazo[4,5-c]pyridin-6-amine,
N-(2-ethyl-4-(thiazol-5-yl)phenyl)-N,1-dimethyl-1H-imidazo[4,5-c]pyridin-6-amine,
3-ethyl-4-(methyl(1-methyl-1H-imidazo[4,5-c]pyridin-6-yl)amino)-N-(1-methylpiperidin-3-yl)benzamide,
N-(4-(3,6-dihydro-2H-pyran-4-yl)-2-ethylphenyl)-N,1-dimethyl-1H-imidazo[4,5-c]pyridin-6-amine,
tert-butyl 4-(3-ethyl-4-(methyl(1-methyl-1H-imidazo[4,5-c]pyridin-6-yl)amino)phenyl)-5,6-dihydropyridine-1 (2H)-carboxylate,
N-(4-(4-chloropyridin-3-yl)-2-ethylphenyl)-N,1-dimethyl-1H-imidazo[4,5-c]pyridin-6-amine,
3-(3-(3-ethyl-4-(methyl(1-methyl-1H-imidazo[4,5-c]pyridin-6-yl)amino)phenyl)-1,2,4-oxadiazol-5-yl)propanenitrile,
N-(2-ethyl-4-(5-(methoxymethyl)-1,2,4-oxadiazol-3-yl)phenyl)-N,1-dimethyl-1H-imidazo[4,5-c]pyridin-6-amine,
1-(4-(3-ethyl-4-(methyl(1-methyl-1H-imidazo[4,5-c]pyridin-6-yl)amino)phenyl)-5,6-dihydropyridin-1 (2H)-yl)propan-1-one,
1-(3-ethyl-4-(methyl(1-methyl-1H-imidazo[4,5-c]pyridin-6-yl)amino)phenyl)-2,2,2-trifluoro ethanol,
3-ethyl-N-(2-hydroxyethyl)-4-(methyl(1-methyl-1H-imidazo[4,5-c]pyridin-6-yl)amino)benzamide,
3-ethyl-N-(2-hydroxy-2-methylpropyl)-4-(methyl(1-methyl-1H-imidazo[4,5-c]pyridin-6-yl)amino)benzamide,
3′-ethyl-4′-(methyl(1-methyl-1H-imidazo[4,5-c]pyridin-6-yl)amino)biphenyl-4-carbonitrile,
3′-ethyl-4′-(methyl(1-methyl-1H-imidazo[4,5-c]pyridin-6-yl)amino)biphenyl-3-carbonitrile,
N-(3-ethylbiphenyl-4-yl)-N,1-dimethyl-1H-imidazo[4,5-c]pyridin-6-amine,
3-ethyl-4-(methyl(1-methyl-1H-imidazo[4,5-c]pyridin-6-yl)amino)-NA 1-methylpiperidin-2-yl)methyl)benzamide,
N-(4-cyano-1-methylpiperidin-4-yl)-3-ethyl-4-(methyl(1-methyl-1H-imidazo[4,5-c]pyridin-6-yl)amino)benzamide,
N-(2-ethyl-4-((oxetan-3-ylamino)methyl)phenyl)-N,1-dimethyl-1H-imidazo[4,5-c]pyridin-6-amine,
1-(4-(3-ethyl-4-(methyl(1-methyl-1H-imidazo[4,5-c]pyridin-6-yl)amino)phenyl)-5,6-dihydropyridin-1 (2H)-yl)ethanone,
1-(3-ethyl-4-(methyl(1-methyl-1H-imidazo[4,5-c]pyridin-6-yl)amino)benzylamino)-2-methylpropan-2-ol,

tert-butyl 3-(3-ethyl-4-(methyl(1-methyl-1H-imidazo[4,5-c]pyridin-6-yl)amino)benzamido)azetidine-1-carboxylate,
3-ethyl-4-(methyl(1-methyl-1H-imidazo[4,5-c]pyridin-6-yl)amino)-N-(oxetan-3-yl)benzamide,
N-ethyl-4-(3-ethyl-4-(methyl(1-methyl-1H-imidazo[4,5-c]pyridin-6-yl)amino)phenyl)-5,6-dihydropyridine-1 (2H)-carboxamide,
N-(2-ethyl-4-((2-methyl-1H-imidazol-1-yl)methyl)phenyl)-N,1-dimethyl-1H-imidazo[4,5-c]pyridin-6-amine,
N-(azetidin-3-yl)-3-ethyl-4-(methyl(1-methyl-1H-imidazo[4,5-c]pyridin-6-yl)amino)benzamide,
3-ethyl-4-(methyl(1-methyl-1H-imidazo[4,5-c]pyridin-6-yl)amino)-N-(1-(methylsulfonyl)azetidin-3-yl)benzamide,
N-(2-ethyl-4-(1-(methylsulfonyl)-1,2,3,6-tetrahydropyridin-4-yl)phenyl)-N,1-dimethyl-1H-imidazo[4,5-c]pyridin-6-amine,
N-(2-ethyl-4-(5-methyl-1,3,4-oxadiazol-2-yl)phenyl)-N,1-dimethyl-1H-imidazo[4,5-c]pyridin-6-amine,
N-(2-ethyl-4-(3-methyl-1,2,4-oxadiazol-5-yl)phenyl)-N,1-dimethyl-1H-imidazo[4,5-c]pyridin-6-amine,
N-cyano-3-ethyl-4-(methyl(1-methyl-1H-imidazo[4,5-c]pyridin-6-yl)amino)benzamide, tert-butyl 4-(3-ethyl-4-(methyl(1-methyl-1H-imidazo[4,5-c]pyridin-6-yl)amino)phenyl)piperidine-1-carboxylate,
N-(4-(aminomethyl)-2-ethylphenyl)-N,1-dimethyl-1H-imidazo[4,5-c]pyridin-6-amine,
N-(3-ethyl-4-(methyl(1-methyl-1H-imidazo[4,5-c]pyridin-6-yl)amino)benzyl)acetamide,
N-(2-ethyl-4-(5-methyl-1H-1,2,4-triazol-3-yl)phenyl)-N,1-dimethyl-1H-imidazo[4,5-c]pyridin-6-amine,
N-(2-ethyl-4-(2-morpholinopyrimidin-5-yl)phenyl)-N,1-dimethyl-1H-imidazo[4,5-c]pyridin-6-amine,
3-ethyl-4-(methyl(1-methyl-1H-imidazo[4,5-c]pyridin-6-yl)amino)benzic acid,
N-(2-ethyl-4-(tetrahydro-2H-pyran-4-yl)phenyl)-N,1-dimethyl-1H-imidazo[4,5-c]pyridin-6-amine,
N-(2-ethyl-4-(piperidin-4-yl)phenyl)-N,1-dimethyl-1H-imidazo[4,5-c]pyridin-6-amine,
N-(4-(5-cyclopropyl-1H-1,2,4-triazol-3-yl)-2-ethylphenyl)-N,1-dimethyl-1H-imidazo[4,5-c]pyridin-6-amine,
5-(3-ethyl-4-(methyl(1-methyl-1H-imidazo[4,5-c]pyridin-6-yl)amino)phenyl)nicotinonitrile,
N-(3-ethyl-4-(methyl(1-methyl-1H-imidazo[4,5-c]pyridin-6-yl)amino)benzyl)methanesulfonamide,
methyl 3-ethyl-4-(methyl(1-methyl-1H-imidazo[4,5-c]pyridin-6-yl)amino)benzylcarbamate,
1-ethyl-3-(3-ethyl-4-(methyl(1-methyl-1H-imidazo[4,5-c]pyridin-6-yl)amino)benzyl)urea,
5-(3-ethyl-4-(methyl(1-methyl-1H-imidazo[4,5-c]pyridin-6-yl)amino)phenyl)-1,3,4-thiadiazol-2-amine,
N-(2-ethyl-4-(5-(trifluoromethyl)pyridin-3-yl)phenyl)-N,1-dimethyl-1H-imidazo[4,5-c]pyridin-6-amine,
3-(4-(3-ethyl-4-(methyl(1-methyl-1H-imidazo[4,5-c]pyridin-6-yl)amino)phenyl)-5,6-dihydropyridin-1 (2H)-yl)-3-oxopropanenitrile,
4-(4-(3-ethyl-4-(methyl(1-methyl-1H-imidazo[4,5-c]pyridin-6-yl)amino)phenyl)-5,6-dihydropyridin-1 (2H)-yl)-4-oxobutanenitrile,
1-(4-(3-ethyl-4-(methyl(1-methyl-1H-imidazo[4,5-c]pyridin-6-yl)amino)phenyl)-5,6-dihydropyridin-1 (2H)-yl)-2,2-difluoroethanone,
1-(4-(3-ethyl-4-(methyl(1-methyl-1H-imidazo[4,5-c]pyridin-6-yl)amino)phenyl)-1,2,3,6-tetrahydropyridine-1-carbonyl)cyclopropanecarbonitrile,
1-(4-(3-ethyl-4-(methyl(1-methyl-1H-imidazo[4,5-c]pyridin-6-yl)amino)phenyl)-5,6-dihydropyridin-1 (2H)-yl)-2-methoxyethanone,
1-(4-(3-ethyl-4-(methyl(1-methyl-1H-imidazo[4,5-c]pyridin-6-yl)amino)phenyl)-5,6-dihydropyridin-1 (2H)-yl)-3-hydroxy-3-methylbutan-1-one,
1-(4-(3-ethyl-4-(methyl(1-methyl-1H-imidazo[4,5-c]pyridin-6-yl)amino)phenyl)-5,6-dihydropyridin-1 (2H)-yl)-2-hydroxypropan-1-one,
4-(3-ethyl-4-(methyl(1-methyl-1H-imidazo[4,5-c]pyridin-6-yl)amino)phenyl)-N-(1-methylcyclopropyl)-5,6-dihydropyridine-1 (2H)-carboxamide,
N-cyano-4-(3-ethyl-4-(methyl(1-methyl-1H-imidazo[4,5-c]pyridin-6-yl)amino)phenyl)-5,6-dihydropyridine-1 (2H)-carboxamide,
4-(3-ethyl-4-(methyl(1-methyl-1H-imidazo[4,5-c]pyridin-6-yl)amino)phenyl)-N-methyl-5,6-dihydropyridine-1 (2H)-carboxamide, azetidin-1-yl(4-(3-ethyl-4-(methyl(1-methyl-1H-imidazo[4,5-c]pyridin-6-yl)amino)phenyl)-5,6-dihydropyridin-1 (2H)-yl)methanone,
N-ethyl-4-(3-ethyl-4-(methyl(1-methyl-1H-imidazo[4,5-c]pyridin-6-yl)amino)phenyl)-N-methyl-5,6-dihydropyridine-1 (2H)-carboxamide,
N-(cyanomethyl)-4-(3-ethyl-4-(methyl(1-methyl-1H-imidazo[4,5-c]pyridin-6-yl)amino)phenyl)-N-methyl-5,6-dihydropyridine-1 (2H)-carboxamide,
N-(2-ethyl-4-(1-(4-methoxybenzyl)-1H-pyrazol-4-yl)phenyl)-N,1-dimethyl-1H-imidazo[4,5-c]pyridin-6-amine,
2-(4-(3-ethyl-4-(methyl(1-methyl-1H-imidazo[4,5-c]pyridin-6-yl)amino)phenyl)-1H-pyrazol-1-yl)acetonitrile,
N-(2-ethyl-4-(1H-pyrazol-4-yl)phenyl)-N,1-dimethyl-1H-imidazo[4,5-c]pyridin-6-amine,
N-(2-ethyl-4-(2-(methylamino)pyrimidin-5-yl)phenyl)-N,1-dimethyl-1H-imidazo[4,5-c]pyridin-6-amine,
N-(2-ethyl-4-(2-(oxetan-3-yloxy)pyrimidin-5-yl)phenyl)-N,1-dimethyl-1H-imidazo[4,5-c]pyridin-6-amine,
N-ethyl-4-(3-ethyl-4-(methyl(1-methyl-1H-imidazo[4,5-c]pyridin-6-yl)amino)phenyl)piperidine-1-carboxamide,
N-(2-ethyl-4-(1-(methylsulfonyl)piperidin-4-yl)phenyl)-N,1-dimethyl-1H-imidazo[4,5-c]pyridin-6-amine,
4-(3-ethyl-4-(methyl(1-methyl-1H-imidazo[4,5-c]pyridin-6-yl)amino)phenyl)-N-(oxetan-3-yl)-5,6-dihydropyridine-1 (2H)-carboxamide, cyanomethyl 4-(3-ethyl-4-(methyl(1-methyl-1H-imidazo[4,5-c]pyridin-6-yl)amino)phenyl)-5,6-dihydropyridine-1 (2H)-carboxylate,
2-(5-(3-ethyl-4-(methyl(1-methyl-1H-imidazo[4,5-c]pyridin-6-yl)amino)phenyl)pyrimidin-2-yloxy)acetonitrile,
N-(2-ethyl-4-(5-(methylsulfonyl)pyridin-3-yl)phenyl)-N,1-dimethyl-1H-imidazo[4,5-c]pyridin-6-amine,
N-(2-ethyl-4-(1-(2-(pyrrolidin-1-yl)ethyl)-1H-pyrazol-4-yl)phenyl)-N,1-dimethyl-1H-imidazo[4,5-c]pyridin-6-amine,
2-(4-(3-ethyl-4-(methyl(1-methyl-1H-imidazo[4,5-c]pyridin-6-yl)amino)phenyl)-1H-pyrazol-1-yl)-N,N-dimethylacetamide,
1-(4-(3-ethyl-4-(methyl(1-methyl-1H-imidazo[4,5-c]pyridin-6-yl)amino)phenyl)-1H-pyrazol-1-yl)-3,3-dimethylbutan-2-one,
2-(4-(3-ethyl-4-(methyl(1-methyl-1H-imidazo[4,5-c]pyridin-6-yl)amino)phenyl)-1H-pyrazol-1-yl)acetamide,
1-(4-(3-ethyl-4-(methyl(1-methyl-1H-imidazo[4,5-c]pyridin-6-yl)amino)phenyl)piperidin-1-yl)propan-1-one,
3-(4-(3-ethyl-4-(methyl(1-methyl-1H-imidazo[4,5-c]pyridin-6-yl)amino)phenyl)piperidin-1-yl)-3-oxopropanenitrile,
1-(4-(3-ethyl-4-(methyl(1-methyl-1H-imidazo[4,5-c]pyridin-6-yl)amino)phenyl)piperidin-1-yl)-2,2-difluoro ethanone,
1-(4-(3-ethyl-4-(methyl(1-methyl-1H-imidazo[4,5-c]pyridin-6-yl)amino)phenyl)piperidin-1-yl)-2-methoxyethanone,
N-(3-ethyl-4-(methyl(1-methyl-1H-imidazo[4,5-c]pyridin-6-yl)amino)benzyl)cyclopropanesulfonamide,
1-cyano-N-(3-ethyl-4-(methyl(1-methyl-1H-imidazo[4,5-c]pyridin-6-yl)amino)benzyl)methanesulfonamide,
(4-(3-ethyl-4-(methyl(1-methyl-1H-imidazo[4,5-c]pyridin-6-yl)amino)phenyl)-5,6-dihydropyridin-1 (2H)-yl)(1-hydroxycyclopropyl)methanone,
1-(4-(3-ethyl-4-(methyl(1-methyl-1H-imidazo[4,5-c]pyridin-6-yl)amino)phenyl)-5,6-dihydropyridin-1 (2H)-yl)-3,3,3-trifluoro-2-hydroxypropan-1-one,
1-(4-(3-ethyl-4-(methyl(1-methyl-1H-imidazo[4,5-c]pyridin-6-yl)amino)phenyl)-5,6-dihydropyridin-1 (2H)-yl)-2-hydroxy-2-methylpropan-1-one,
N-(2-ethyl-4-(1-(2,2,2-trifluoroethylsulfonyl)-1,2,3,6-tetrahydropyridin-4-yl)phenyl)-N,1-dimethyl-1H-imidazo[4,5-c]pyridin-6-amine,
N-(2-ethyl-4-(1-(piperidin-1-ylsulfonyl)-1,2,3,6-tetrahydropyridin-4-yl)phenyl)-N,1-dimethyl-1H-imidazo[4,5-c]pyridin-6-amine,
4-(3-ethyl-4-(methyl(1-methyl-1H-imidazo[4,5-c]pyridin-6-yl)amino)phenyl)-N-(1,3,4-thiadiazol-2-yl)-5,6-dihydropyridine-1 (2H)-carboxamide,
1-(4-(3-ethyl-4-(methyl(1-methyl-1H-imidazo[4,5-c]pyridin-6-yl)amino)phenyl)piperidin-1-yl)-2-hydroxypropan-1-one,
N-(2-ethyl-4-(1-(3-methoxybenzyl)-1H-pyrazol-4-yl)phenyl)-N,1-dimethyl-1H-imidazo[4,5-c]pyridin-6-amine,
N-(2-ethyl-4-(1-(4-methylbenzyl)-1H-pyrazol-4-yl)phenyl)-N,1-dimethyl-1H-imidazo[4,5-c]pyridin-6-amine,
N-(2-ethyl-4-(1-(4-fluorobenzyl)-1H-pyrazol-4-yl)phenyl)-N,1-dimethyl-1H-imidazo[4,5-c]pyridin-6-amine,
N-(4-(1-(4-chlorobenzyl)-1H-pyrazol-4-yl)-2-ethylphenyl)-N,1-dimethyl-1H-imidazo[4,5-c]pyridin-6-amine,
N-(2-ethyl-4-(1-propyl-1H-pyrazol-4-yl)phenyl)-N,1-dimethyl-1H-imidazo[4,5-c]pyridin-6-amine,
N-(2-ethyl-4-(1-ethyl-1H-pyrazol-4-yl)phenyl)-N,1-dimethyl-1H-imidazo[4,5-c]pyridin-6-amine,
N-(2-ethyl-4-(1-((3-methyloxetan-3-yl)methyl)-1H-pyrazol-4-yl)phenyl)-N,1-dimethyl-1H-imidazo[4,5-c]pyridin-6-amine,
N-(3-ethyl-4-(methyl(1-methyl-1H-imidazo[4,5-c]pyridin-6-yl)amino)benzyl)piperidine-1-sulfonamide,
N-(3-ethyl-4-(methyl(1-methyl-1H-imidazo[4,5-c]pyridin-6-yl)amino)benzyl)-2,2,2-trifluoroethanesulfonamide,
N-(3-ethyl-4-(methyl(1-methyl-1H-imidazo[4,5-c]pyridin-6-yl)amino)benzyl)propane-2-sulfonamide,
N-(2-ethyl-4-(1-(isopropylsulfonyl)-1,2,3,6-tetrahydropyridin-4-yl)phenyl)-N,1-dimethyl-1H-imidazo[4,5-c]pyridin-6-amine,
2-(4-(3-ethyl-4-(methyl(1-methyl-1H-imidazo[4,5-c]pyridin-6-yl)amino)phenyl)-5,6-dihydropyridin-1 (2H)-ylsulfonyl)acetonitrile,
(R)-1-(4-(3-ethyl-4-(methyl(1-methyl-1H-imidazo[4,5-c]pyridin-6-yl)amino)phenyl)-5,6-dihydropyridin-1 (2H)-yl)-2-hydroxypropan-1-one,
tert-butyl 1-(4-(3-ethyl-4-(methyl(1-methyl-1H-imidazo[4,5-c]pyridin-6-yl)amino)phenyl)-5,6-dihydropyridin-1 (2H)-yl)-1-oxopropan-2-ylcarbamate,
1-(4-(3-ethyl-4-(methyl(1-methyl-1H-imidazo[4,5-c]pyridin-6-yl)amino)phenyl)-5,6-dihydropyridin-1 (2H)-yl)-2-methoxypropan-1-one,
(S)-1-(4-(3-ethyl-4-(methyl(1-methyl-1H-imidazo[4,5-c]pyridin-6-yl)amino)phenyl)-5,6-dihydropyridin-1 (2H)-yl)-2-hydroxypropan-1-one,
tert-butyl 3-ethyl-4-(methyl(1-methyl-1H-imidazo[4,5-c]pyridin-6-yl)amino)benzylcarbamate,
N-(3-ethyl-4-(methyl(1-methyl-1H-imidazo[4,5-c]pyridin-6-yl)amino)benzyl)ethanesulfonamide,
N-(3-ethyl-4-(methyl(1-methyl-1H-imidazo[4,5-c]pyridin-6-yl)amino)benzyl)-2-methoxyacetamide,
2-cyano-N-(3-ethyl-4-(methyl(1-methyl-1H-imidazo[4,5-c]pyridin-6-yl)amino)benzyl)acetamide,
N-(3-ethyl-4-(methyl(1-methyl-1H-imidazo[4,5-c]pyridin-6-yl)amino)benzyl)-2,2-difluoroacetamide,
4-(3-ethyl-4-(methyl(1-methyl-1H-imidazo[4,5-c]pyridin-6-yl)amino)phenyl)-N-(1,2,4-thiadiazol-5-yl)-5,6-dihydropyridine-1 (2H)-carboxamide,
1-{3-Ethyl-4-[methyl-(1-methyl-1H-imidazo[4,5-c]pyridin-6-yl)-amino]-benzyl}-3-cyano-urea,
(R)—N-(2-ethyl-4-(1-(methylsulfonyl)pyrrolidin-3-yloxy)phenyl)-N,1-dimethyl-1H-imidazo[4,5-c]pyridin-6-amine,
(S)—N-(2-ethyl-4-(1-(methylsulfonyl)pyrrolidin-3-yloxy)phenyl)-N,1-dimethyl-1H-imidazo[4,5-c]pyridin-6-amine,
N-(3-ethyl-4-(methyl(1-methyl-1H-imidazo[4,5-c]pyridin-6-yl)amino)benzyl)-N-methylmethanesulfonamide,
[4-(1,1-Dioxoisothiazolidin-2-ylmethyl)-2-ethyl-phenyl]-methyl-(1-methyl-1H-imidazo[4,5-c]pyridin-6-yl)-amine,
N-(4-methoxy-6-(1-methyl-1H-pyrazol-4-yl)pyridin-3-yl)-N,1-dimethyl-1H-imidazo[4,5-c]pyridin-6-amine,
N-(4-methoxy-6-(thiophen-2-yl)pyridin-3-yl)-N,1-dimethyl-1H-imidazo[4,5-c]pyridin-6-amine,
3-(4-(4-methoxy-5-(methyl(1-methyl-1H-imidazo[4,5-c]pyridin-6-yl)amino)pyridin-2-yl)piperidin-1-yl)-3-oxopropanenitrile,
4-(ethyl(1-methyl-1H-imidazo[4,5-c]pyridin-6-yl)amino)-3-fluorobenzonitrile,
3-fluoro-4-(methyl(1-methyl-1H-imidazo[4,5-c]pyridin-6-yl)amino)benzonitrile, tert-butyl 1-(3-fluoro-4-(methyl(1-methyl-1H-imidazo[4,5-c]pyridin-6-yl)amino)phenyl)ethylcarbamate,
N-(3-fluoro-4-(methyl(1-methyl-1H-imidazo[4,5-c]pyridin-6-yl)amino)benzyl)methanesulfonamide,
1-(3-ethyl-4-(methyl(1-methyl-1H-imidazo[4,5-c]pyridin-6-yl)amino)benzyl)-3-methylurea,
N-(2-ethyl-4-methoxyphenyl)-N,1-dimethyl-1H-imidazo[4,5-c]pyridin-6-amine,
N-(4-ethoxy-2-ethylphenyl)-N,1-dimethyl-1H-imidazo[4,5-c]pyridin-6-amine, tert-butyl 4-(4-methoxy-5-(methyl(1-methyl-1H-imidazo[4,5-c]pyridin-6-yl)amino)pyridin-2-yl)piperidine-1-carboxylate,
N-(2-(difluoromethoxy)-4-(1-methyl-1H-pyrazol-4-yl)phenyl)-N,1-dimethyl-1H-imidazo[4,5-c]pyridin-6-amine,
6-(4-methoxybenzyl)-1-methyl-1H-imidazo[4,5-c]pyridine,
(1′-Methanesulfonyl-4-methoxy-1′,2′,3′,4′,5′,6′-hexahydro-[2,4]bipyridinyl-5-yl)-methyl-(1-methyl-1H-imidazo[4,5-c]pyridin-6-yl)-amine,
[2-Fluoro-4-(1-methanesulfonyl-azetidin-3-yl)-phenyl]-methyl-(1-methyl-1H-imidazo[4,5-c]pyridin-6-yl)-amine,
1-(3-{3-Fluoro-4-[methyl-(1-methyl-1H-imidazo[4,5-c]pyridin-6-yl)-amino]-phenyl}-azetidin-1-yl)-ethanone,
4-{3-Fluoro-4-[methyl-(1-methyl-1H-imidazo[4,5-c]pyridin-6-yl)-amino]-phenyl}-piperidine-1-carboxylic acid tert-butyl ester,
[4-Ethyl-6-(1-methyl-1H-pyrazol-4-yl)-pyridin-3-yl]-methyl-(1-methyl-1H-imidazo[4,5-c]pyridin-6-yl)-amine,
4-{3-Difluoromethoxy-4-[methyl-(1-methyl-1H-imidazo[4,5-c]pyridin-6-yl)-amino]-phenyl}-3,6-dihydro-2H-pyridine-1-carboxylic acid tert-butyl ester,
4-[Cyclopropylmethyl-(1-methyl-1H-imidazo[4,5-c]pyridin-6-yl)-amino]-3-fluoro-benzonitrile,
{3-Fluoro-4-[methyl-(1-methyl-1H-imidazo[4,5-c]pyridin-6-yl)-amino]-benzyl}-carbamic acid methyl ester,
(2,2-Difluoro-ethyl)-(2-ethyl-phenyl)-(1-methyl-1H-imidazo[4,5-c]pyridin-6-yl)-amine,
3-[(2-Ethyl-phenyl)-(1-methyl-1-imidazo[4,5-c]pyridin-6-yl)-amino]-propionitrile,
4-{3-Difluoromethoxy-4-[methyl-(1-methyl-1H-imidazo[4,5-c]pyridin-6-yl)-amino]-phenyl}-piperidine-1-carboxylic acid tert-butyl ester,
[2-Difluoromethoxy-4-(1-methanesulfonyl-piperidin-4-yl)-phenyl]-methyl-(1-methyl-1H-midazo[4,5-c]pyridin-6-yl)-amine,
(4-Ethyl-6-thiophen-3-yl-pyridin-3-yl)-methyl-(1-methyl-1H-imidazo[4,5-c]pyridin-6-yl)-amine,
(4-Ethyl-6-thiophen-2-yl-pyridin-3-yl)-methyl-(1-methyl-1H-imidazo[4,5-c]pyridin-6-yl)-amine,
[2-Ethyl-4-(1-methanesulfonyl-azetidin-3-yl)-phenyl]-methyl-(1-methyl-1H-imidazo[4,5-c]pyridin-6-yl)-amine,
3-{3-Ethyl-4-[methyl-(1-methyl-1H-imidazo[4,5-c]pyridin-6-yl)-amino]-benzylamino}-pyrrolidine-1-carboxylic acid tert-butyl ester,
Cyclopropanesulfonic acid {3-ethyl-4-[methyl-(1-methyl-1H-imidazo[4,5-c]pyridin-6-yl)-amino]-benzyl}-methyl-amide,
[2-Ethyl-4-(pyrrolidin-3-ylaminomethyl)-phenyl]-methyl-(1-methyl-1H-imidazo[4,5-c]pyridin-6-yl)-amine,
Ethanesulfonic acid {3-ethyl-4-[methyl-(1-methyl-1H-imidazo[4,5-c]pyridin-6-yl)-amino]-benzyl}-methyl-amide,
2-(3-ethyl-4-(methyl(1-methyl-1H-imidazo[4,5-c]pyridin-6-yl)amino)phenoxy)acetonitrile,
1-cyclopropyl-N-(2-ethylphenyl)-N-methyl-1H-imidazo[4,5-c]pyridin-6-amine,
1-ethyl-N-(2-ethylphenyl)-N-methyl-1H-imidazo[4,5-c]pyridin-6-amine, and
6-(2-Ethyl-phenoxy)-1-methyl-1H-imidazo[4,5-c]pyridineN-(2-(difluoromethoxy)-4-(1-methyl-1H-pyrazol-4-yl)phenyl)-N,1-dimethyl-1H-imidazo[4,5-c]pyridin-6-amine.

In one embodiment, a compound of the invention is according to Formula I, wherein the compound is selected from:
N-(2-ethyl-4-(1-(piperidin-1-ylsulfonyl)-1,2,3,6-tetrahydropyridin-4-yl)phenyl)-N,1-dimethyl-1H-imidazo[4,5-c]pyridin-6-amine,
4-(3-ethyl-4-(methyl(1-methyl-1H-imidazo[4,5-c]pyridin-6-yl)amino)phenyl)-N-(1,3,4-thiadiazol-2-yl)-5,6-dihydropyridine-1 (2H)-carboxamide,
1-(4-(3-ethyl-4-(methyl(1-methyl-1H-imidazo[4,5-c]pyridin-6-yl)amino)phenyl)piperidin-1-yl)-2-hydroxypropan-1-one,
N-(2-ethyl-4-(1-(3-methoxybenzyl)-1H-pyrazol-4-yl)phenyl)-N,1-dimethyl-1H-imidazo[4,5-c]pyridin-6-amine,
N-(2-ethyl-4-(1-(4-methylbenzyl)-1H-pyrazol-4-yl)phenyl)-N,1-dimethyl-1H-imidazo[4,5-c]pyridin-6-amine,
N-(2-ethyl-4-(1-(4-fluorobenzyl)-1H-pyrazol-4-yl)phenyl)-N,1-dimethyl-1H-imidazo[4,5-c]pyridin-6-amine,
N-(4-(1-(4-chlorobenzyl)-1H-pyrazol-4-yl)-2-ethylphenyl)-N,1-dimethyl-1H-imidazo[4,5-c]pyridin-6-amine,
N-(2-ethyl-4-(1-propyl-1H-pyrazol-4-yl)phenyl)-N,1-dimethyl-1H-imidazo[4,5-c]pyridin-6-amine,
N-(2-ethyl-4-(1-ethyl-1H-pyrazol-4-yl)phenyl)-N,1-dimethyl-1H-imidazo[4,5-c]pyridin-6-amine,
N-(2-ethyl-4-(1-((3-methyloxetan-3-yl)methyl)-1H-pyrazol-4-yl)phenyl)-N,1-dimethyl-1H-imidazo[4,5-c]pyridin-6-amine,
N-(3-ethyl-4-(methyl(1-methyl-1H-imidazo[4,5-c]pyridin-6-yl)amino)benzyl)piperidine-1-sulfonamide,
N-(3-ethyl-4-(methyl(1-methyl-1H-imidazo[4,5-c]pyridin-6-yl)amino)benzyl)-2,2,2-trifluoroethanesulfonamide,
N-(3-ethyl-4-(methyl(1-methyl-1H-imidazo[4,5-c]pyridin-6-yl)amino)benzyl)propane-2-sulfonamide,
N-(2-ethyl-4-(1-(isopropylsulfonyl)-1,2,3,6-tetrahydropyridin-4-yl)phenyl)-N,1-dimethyl-1H-imidazo[4,5-c]pyridin-6-amine,
2-(4-(3-ethyl-4-(methyl(1-methyl-1H-imidazo[4,5-c]pyridin-6-yl)amino)phenyl)-5,6-dihydropyridin-1 (2H)-ylsulfonyl)acetonitrile,
(R)-1-(4-(3-ethyl-4-(methyl(1-methyl-1H-imidazo[4,5-c]pyridin-6-yl)amino)phenyl)-5,6-dihydropyridin-1 (2H)-yl)-2-hydroxypropan-1-one,
tert-butyl 1-(4-(3-ethyl-4-(methyl(1-methyl-1H-imidazo[4,5-c]pyridin-6-yl)amino)phenyl)-5,6-dihydropyridin-1 (2H)-yl)-1-oxopropan-2-ylcarbamate,
1-(4-(3-ethyl-4-(methyl(1-methyl-1H-imidazo[4,5-c]pyridin-6-yl)amino)phenyl)-5,6-dihydropyridin-1 (2H)-yl)-2-methoxypropan-1-one,
(S)-1-(4-(3-ethyl-4-(methyl(1-methyl-1H-imidazo[4,5-c]pyridin-6-yl)amino)phenyl)-5,6-dihydropyridin-1 (2H)-yl)-2-hydroxypropan-1-one,
tert-butyl 3-ethyl-4-(methyl(1-methyl-1H-imidazo[4,5-c]pyridin-6-yl)amino)benzylcarbamate,
N-(3-ethyl-4-(methyl(1-methyl-1H-imidazo[4,5-c]pyridin-6-yl)amino)benzyl)ethanesulfonamide,
N-(3-ethyl-4-(methyl(1-methyl-1H-imidazo[4,5-c]pyridin-6-yl)amino)benzyl)-2-methoxyacetamide,
2-cyano-N-(3-ethyl-4-(methyl(1-methyl-1H-imidazo[4,5-c]pyridin-6-yl)amino)benzyl)acetamide,
N-(3-ethyl-4-(methyl(1-methyl-1H-imidazo[4,5-c]pyridin-6-yl)amino)benzyl)-2,2-difluoroacetamide,
4-(3-ethyl-4-(methyl(1-methyl-1H-imidazo[4,5-c]pyridin-6-yl)amino)phenyl)-N-(1,2,4-thiadiazol-5-yl)-5,6-dihydropyridine-1 (2H)-carboxamide,
1-{3-Ethyl-4-[methyl-(1-methyl-1H-imidazo[4,5-c]pyridin-6-yl)-amino]-benzyl}-3-cyano-urea,
(R)—N-(2-ethyl-4-(1-(methylsulfonyl)pyrrolidin-3-yloxy)phenyl)-N,1-dimethyl-1H-imidazo[4,5-c]pyridin-6-amine,
(S)—N-(2-ethyl-4-(1-(methylsulfonyl)pyrrolidin-3-yloxy)phenyl)-N,1-dimethyl-1H-imidazo[4,5-c]pyridin-6-amine,
N-(3-ethyl-4-(methyl(1-methyl-1H-imidazo[4,5-c]pyridin-6-yl)amino)benzyl)-N-methylmethanesulfonamide,
[4-(1,1-Dioxoisothiazolidin-2-ylmethyl)-2-ethyl-phenyl]-methyl-(1-methyl-1H-imidazo[4,5-c]pyridin-6-yl)-amine,
N-(4-methoxy-6-(1-methyl-1H-pyrazol-4-yl)pyridin-3-yl)-N,1-dimethyl-1H-imidazo[4,5-c]pyridin-6-amine,
N-(4-methoxy-6-(thiophen-2-yl)pyridin-3-yl)-N,1-dimethyl-1H-imidazo[4,5-c]pyridin-6-amine,
3-(4-(4-methoxy-5-(methyl(1-methyl-1H-imidazo[4,5-c]pyridin-6-yl)amino)pyridin-2-yl)piperidin-1-yl)-3-oxopropanenitrile,
4-(ethyl(1-methyl-1H-imidazo[4,5-c]pyridin-6-yl)amino)-3-fluorobenzonitrile,
3-fluoro-4-(methyl(1-methyl-1H-imidazo[4,5-c]pyridin-6-yl)amino)benzonitrile,
tert-butyl 1-(3-fluoro-4-(methyl(1-methyl-1H-imidazo[4,5-c]pyridin-6-yl)amino)phenyl)ethylcarbamate,
N-(3-fluoro-4-(methyl(1-methyl-1H-imidazo[4,5-c]pyridin-6-yl)amino)benzyl)methanesulfonamide,
1-(3-ethyl-4-(methyl(1-methyl-1H-imidazo[4,5-c]pyridin-6-yl)amino)benzyl)-3-methylurea,
N-(2-ethyl-4-methoxyphenyl)-N,1-dimethyl-1H-imidazo[4,5-c]pyridin-6-amine,
N-(4-ethoxy-2-ethylphenyl)-N,1-dimethyl-1H-imidazo[4,5-c]pyridin-6-amine, tert-butyl 4-(4-methoxy-5-(methyl(1-methyl-1H-imidazo[4,5-c]pyridin-6-yl)amino)pyridin-2-yl)piperidine-1-carboxylate,
N-(2-(difluoromethoxy)-4-(1-methyl-1H-pyrazol-4-yl)phenyl)-N,1-dimethyl-1H-imidazo[4,5-c]pyridin-6-amine,
6-(4-methoxybenzyl)-1-methyl-1H-imidazo[4,5-c]pyridine,
(1′-Methanesulfonyl-4-methoxy-1′,2′,3′,4′,5′,6′-hexahydro-[2,4]bipyridinyl-5-yl)-methyl-(1-methyl-1H-imidazo[4,5-c]pyridin-6-yl)-amine,
[2-Fluoro-4-(1-methanesulfonyl-azetidin-3-yl)-phenyl]-methyl-(1-methyl-1H-imidazo[4,5-c]pyridin-6-yl)-amine,
1-(3-{3-Fluoro-4-[methyl-(1-methyl-1H-imidazo[4,5-c]pyridin-6-yl)-amino]-phenyl}-azetidin-1-yl)-ethanone,
4-{3-Fluoro-4-[methyl-(1-methyl-1H-imidazo[4,5-c]pyridin-6-yl)-amino]-phenyl}-piperidine-1-carboxylic acid tert-butyl ester,
[4-Ethyl-6-(1-methyl-1-pyrazol-4-yl)-pyridin-3-yl]-methyl-(1-methyl-1H-imidazo[4,5-c]pyridin-6-yl)-amine,
4-{3-Difluoromethoxy-4-[methyl-(1-methyl-1H-imidazo[4,5-c]pyridin-6-yl)-amino]-phenyl}-3,6-dihydro-2H-pyridine-1-carboxylic acid tert-butyl ester,
4-[Cyclopropylmethyl-(1-methyl-1H-imidazo[4,5-c]pyridin-6-yl)-amino]-3-fluoro-benzonitrile,
{3-Fluoro-4-[methyl-(1-methyl-1H-imidazo[4,5-c]pyridin-6-yl)-amino]-benzyl}-carbamic acid methyl ester,
(2,2-Difluoro-ethyl)-(2-ethyl-phenyl)-(1-methyl-1H-imidazo[4,5-c]pyridin-6-yl)-amine,
3-[(2-Ethyl-phenyl)-(1-methyl-1-imidazo[4,5-c]pyridin-6-yl)-amino]-propionitrile,
4-{3-Difluoromethoxy-4-[methyl-(1-methyl-1H-imidazo[4,5-c]pyridin-6-yl)-amino]-phenyl}-piperidine-1-carboxylic acid tert-butyl ester,
[2-Difluoromethoxy-4-(1-methanesulfonyl-piperidin-4-yl)-phenyl]-methyl-(1-methyl-1H-midazo[4,5-c]pyridin-6-yl)-amine,
(4-Ethyl-6-thiophen-3-yl-pyridin-3-yl)-methyl-(1-methyl-1H-imidazo[4,5-c]pyridin-6-yl)-amine,
(4-Ethyl-6-thiophen-2-yl-pyridin-3-yl)-methyl-(1-methyl-1H-imidazo[4,5-c]pyridin-6-yl)-amine,
[2-Ethyl-4-(1-methanesulfonyl-azetidin-3-yl)-phenyl]-methyl-(1-methyl-1H-imidazo[4,5-c]pyridin-6-yl)-amine,
3-{3-Ethyl-4-[methyl-(1-methyl-1H-imidazo[4,5-c]pyridin-6-yl)-amino]-benzylamino}-pyrrolidine-1-carboxylic acid tert-butyl ester,
Cyclopropanesulfonic acid {3-ethyl-4-[methyl-(1-methyl-1H-imidazo[4,5-c]pyridin-6-yl)-amino]-benzyl}-methyl-amide,
[2-Ethyl-4-(pyrrolidin-3-ylaminomethyl)-phenyl]-methyl-(1-methyl-1H-imidazo[4,5-c]pyridin-6-yl)-amine,
Ethanesulfonic acid {3-ethyl-4-[methyl-(1-methyl-1H-imidazo[4,5-c]pyridin-6-yl)-amino]-benzyl}-methyl-amide,
2-(3-ethyl-4-(methyl(1-methyl-1H-imidazo[4,5-c]pyridin-6-yl)amino)phenoxy)acetonitrile,
1-cyclopropyl-N-(2-ethylphenyl)-N-methyl-1H-imidazo[4,5-c]pyridin-6-amine,
1-ethyl-N-(2-ethylphenyl)-N-methyl-1H-imidazo[4,5-c]pyridin-6-amine,
6-(2-Ethyl-phenoxy)-1-methyl-1H-imidazo[4,5-c]pyridine,
2-{3-Ethyl-4-[methyl-(1-ethyl-1H-imidazo[4,5-c]pyridin-6-yl)-amino]-phenoxy}-acetamide,
{4-[(1,1-Dioxo-tetrahydro-thiophen-3-ylamino)-methyl]-2-ethyl-phenyl}-methyl-(1-methyl-1H-imidazo[4,5-c]pyridin-6-yl)-amine,
5-{4-Ethyl-5-[methyl-(1-methyl-1-imidazo[4,5-c]pyridin-6-yl)-amino]-pyridin-2-yl}-thiophene-2-carbonitrile,
(5-{4-Ethyl-5-[methyl-(1-ethyl-1H-imidazo[4,5-c]pyridin-6-yl)-amino]-pyridin-2-yl}-thiophen-2-ylmethyl)-carbamic acid tert-butyl ester,
[6-(5-Aminomethyl-thiophen-2-yl)-4-ethyl-pyridin-3-yl]-methyl-(1-methyl-1H-imidazo[4,5-c]pyridin-6-yl)-amine,
[6-(5-Chloro-thiophen-2-yl)-4-ethyl-pyridin-3-yl]-methyl-(1-methyl-1H-imidazo[4,5-c]pyridin-6-yl)-amine,
(2-Ethyl-5-methyl-2H-pyrazol-3-yl)-methyl-(1-methyl-1H-imidazo[4,5-c]pyridin-6-yl)-amine,
(5-Cyclopropyl-2-methyl-2H-pyrazol-3-yl)-methyl-(1-methyl-1H-imidazo[4,5-c]pyridin-6-yl)-amine,
[5-(4-Chloro-phenyl)-2-ethyl-2H-pyrazol-3-yl]-methyl-(1-methyl-1H-imidazo[4,5-c]pyridin-6-yl)-amine,
3-Cyanomethyl-3-{4-ethyl-3-[methyl-(1-methyl-1H-imidazo[4,5-c]pyridin-6-yl)-amino]-pyrazol-1-yl}-azetidine-1-carboxylic acid tert-butyl ester,
(3-{4-Ethyl-3-[methyl-(1-methyl-1-imidazo[4,5-c]pyridin-6-yl)-amino]-pyrazol-1-yl}-azetidin-3-yl)-acetonitrile,
(3-{4-Ethyl-3-[methyl-(1-methyl-1H-imidazo[4,5-c]pyridin-6-yl)-amino]-pyrazol-1-yl}-1-methanesulfonyl-azetidin-3-yl)-acetonitrile,
[4-Ethyl-6-(5-methyl-thiophen-2-yl)-pyridin-3-yl]-methyl-(1-methyl-1H-imidazo[4,5-c]pyridin-6-yl)-amine,
[4-Methoxy-6-(1,3,5-trimethyl-1H-pyrazol-4-yl)-pyridin-3-yl]-methyl-(1-methyl-1H-imidazo[4,5-c]pyridin-6-yl)-amine,
(4-Methoxy-6-morpholin-4-yl-pyridin-3-yl)-methyl-(1-methyl-1H-imidazo[4,5-c]pyridin-6-yl)-amine,
[2-Methoxy-5-(piperidine-1-sulfonyl)-phenyl]-methyl-(1-methyl-1H-imidazo[4,5-c]pyridin-6-yl)-amine,
[2-Methoxy-4-(morpholine-4-sulfonyl)-phenyl]-methyl-(1-methyl-1-imidazo[4,5-c]pyridin-6-yl)-amine,
3-{4-Ethyl-3-[methyl-(1-methyl-1H-imidazo[4,5-c]pyridin-6-yl)-amino]-pyrazol-1-yl}-propionitrile,
N-{4-Ethyl-5-[methyl-(1-methyl-1H-imidazo[4,5-c]pyridin-6-yl)-amino]-pyridin-2-ylmethyl}-N-methyl-methane sulfonamide,
1-{4-Ethyl-5-[methyl-(1-methyl-1H-imidazo[4,5-c]pyridin-6-yl)-amino]-pyridin-2-yl}-pyrrolidin-, 2-one,
N-{5-Ethyl-2-fluoro-4-[methyl-(1-methyl-1H-imidazo[4,5-c]pyridin-6-yl)-amino]-benzyl}-N-methyl-methanesulfonamide,
2,2-Difluoro-N-{3-fluoro-4-[methyl-(1-methyl-1H-imidazo[4,5-c]pyridin-6-yl)-amino]-benzyl}-acetamide,
5-Ethyl-4-[methyl-(1-methyl-1H-imidazo[4,5-c]pyridin-6-yl)-amino]-2-trifluoromethyl-benzonitrile,
3-Ethyl-5-fluoro-4-[methyl-(1-methyl-1H-imidazo[4,5-c]pyridin-6-yl)-amino]-benzonitrile,
N-{3-Ethyl-5-fluoro-4-[methyl-(1-methyl-1H-imidazo[4,5-c]pyridin-6-yl)-amino]-benzyl}-methanesulfonamide,
N-{3-Ethyl-5-fluoro-4-[methyl-(1-methyl-1H-imidazo[4,5-c]pyridin-6-yl)-amino]-benzyl}-C,C-difluoro-methanesulfonamide,
N-{5-Ethyl-2-fluoro-4-[methyl-(1-methyl-1H-imidazo[4,5-c]pyridin-6-yl)-amino]-benzyl}-methanesulfonamide,
N-{5-Ethyl-2-fluoro-4-[methyl-(1-methyl-1H-imidazo[4,5-c]pyridin-6-yl)-amino]-benzyl}-2,2-difluoro-acetamide,
N-{5-Ethyl-2-fluoro-4-[methyl-(1-methyl-1H-imidazo[4,5-c]pyridin-6-yl)-amino]-benzyl}-C,C-difluoro-methanesulfonamide,
[4-Ethyl-6-(1-methanesulfonyl-azetidin-3-yl)-pyridin-3-yl]-methyl-(1-methyl-1H-imidazo[4,5-c]pyridin-6-yl)-amine {6-[1-(Difluoro-methanesulfonyl)-azetidin-3-yl]-4-ethyl-pyridin-3-yl}-methyl-(1-methyl-1H-imidazo[4,5-c]pyridin-6-yl)-amine,
{4-Ethyl-6-[1-(propane-2-sulfonyl)-azetidin-3-yl]-pyridin-3-yl}-methyl-(1-methyl-1H-imidazo[4,5-c]pyridin-6-yl)-amine,
(3-{4-Ethyl-5-[methyl-(1-methyl-1H-imidazo[4,5-c]pyridin-6-yl)-amino]-pyridin-2-yl}-azetidin-1-yl)-acetonitrile,
1-(3-{4-Ethyl-5-[methyl-(1-methyl-1H-imidazo[4,5-c]pyridin-6-yl)-amino]-pyridin-2-yl}-azetidin-1-yl)-ethanone,
1-(3-{4-Ethyl-5-[methyl-(1-methyl-1H-imidazo[4,5-c]pyridin-6-yl)-amino]-pyridin-2-yl}-azetidin-1-yl)-2,2-difluoro-ethanone,
1-(3-{4-Ethyl-5-[methyl-(1-methyl-1H-imidazo[4,5-c]pyridin-6-yl)-amino]-pyridin-2-yl}-azetidin-1-yl)-2,2,2-trifluoro-ethanone,
3-(3-{4-Ethyl-5-[methyl-(1-methyl-1H-imidazo[4,5-c]pyridin-6-yl)-amino]-pyridin-2-yl}-azetidin-1-yl)-3-oxo-propionitrile, 1-(3-{4-Ethyl-5-[methyl-(1-methyl-1H-imidazo[4,5-c]pyridin-6-yl)-amino]-pyridin-2-yl}-azetidin-1-yl)-propan-1-one,
(3-{4-Ethyl-5-[methyl-(1-methyl-1H-imidazo[4,5-c]pyridin-6-yl)-amino]-pyridin-2-yl}-azetidin-1-yl)-(tetrahydro-pyran-4-yl)-methanone,
1-(3-{4-Ethyl-5-[methyl-(1-methyl-1H-imidazo[4,5-c]pyridin-6-yl)-amino]-pyridin-2-yl}-azetidin-1-yl)-4,4,4-trifluoro-butan-1-one,
3-{4-Ethyl-5-[methyl-(1-methyl-1-imidazo[4,5-c]pyridin-6-yl)-amino]-pyridin-2-yl}-azetidine-1-carboxylic acid methyl ester,
(3,3-Difluoro-cyclobutyl)-(3-{4-ethyl-5-[methyl-(1-methyl-1H-imidazo[4,5-c]pyridin-6-yl)-amino]-pyridin-2-yl}-azetidin-1-yl)-methanone,
{4-Ethyl-6-[1-(3,3,3-trifluoro-propyl)-azetidin-3-yl]-pyridin-3-yl}-methyl-(1-methyl-1H-imidazo[4,5-c]pyridin-6-yl)-amine,
{4-Ethyl-6-[1-(2,2,2-trifluoro-ethyl)-azetidin-3-yl]-pyridin-3-yl}-methyl-(1-methyl-1H-imidazo[4,5-c]pyridin-6-yl)-amine,
N-{4-Ethyl-5-[methyl-(1-methyl-1-imidazo[4,5-c]pyridin-6-yl)-amino]-pyridin-2-ylmethyl}-C,C-difluoro-methane sulfonamide,
N-Cyanomethyl-N-{4-ethyl-5-[methyl-(1-methyl-1H-imidazo[4,5-c]pyridin-6-yl)-amino]-pyridin-2-ylmethyl}-methanesulfonamide,
(E)-3-{4-[Methyl-(1-methyl-1H-imidazo[4,5-c]pyridin-6-yl)-amino]-phenyl}-acrylonitrile,
1-(3-{3-Ethyl-4-[methyl-(1-methyl-1H-imidazo[4,5-c]pyridin-6-yl)-amino]-phenyl}-azetidin-1-yl)-2,2-difluoro-ethanone,
1-(3-{3-Ethyl-4-[methyl-(1-methyl-1H-imidazo[4,5-c]pyridin-6-yl)-amino]-phenyl}-azetidin-1-yl)-2,2,2-trifluoro-ethanone,
1-(3-{3-Ethyl-4-[methyl-(1-methyl-1H-imidazo[4,5-c]pyridin-6-yl)-amino]-phenyl}-azetidine-1-carbonyl)-cyclopropanecarbonitrile,
{2-Ethyl-4-[1-(2-methoxy-ethanesulfonyl)-azetidin-3-yl]-phenyl}-methyl-(1-methyl-1H-imidazo[4,5-c]pyridin-6-yl)-amine,
1-(3-{3-Ethyl-4-[methyl-(1-methyl-1H-imidazo[4,5-c]pyridin-6-yl)-amino]-phenyl}-azetidin-1-yl)-2-methoxy-ethanone,
(3-{3-Ethyl-4-[methyl-(1-methyl-1H-imidazo[4,5-c]pyridin-6-yl)-amino]-phenyl}-azetidin-1-yl)-acetonitrile,
{2-Ethyl-4-[1-(2,2,2-trifluoro-ethanesulfonyl)-azetidin-3-yl]-phenyl}-methyl-(1-methyl-1H-imidazo[4,5-c]pyridin-6-yl)-amine,
(3-{3-Ethyl-4-[methyl-(1-methyl-1H-imidazo[4,5-c]pyridin-6-yl)-amino]-phenyl}-azetidine-1-sulfonyl)-acetonitrile,
{4-[1-(Difluoro-methanesulfonyl)-azetidin-3-yl]-2-ethyl-phenyl}-methyl-(1-methyl-1H-imidazo[4,5-c]pyridin-6-yl)-amine,
N-Cyanomethyl-N-{3-fluoro-4-[methyl-(1-methyl-1H-imidazo[4,5-c]pyridin-6-yl)-amino]-benzyl}-methanesulfonamide,
N-{3-Ethyl-5-fluoro-4-[methyl-(1-methyl-1H-imidazo[4,5-c]pyridin-6-yl)-amino]-benzyl}-N-methyl-methanesulfonamide,
N-Cyanomethyl-N-{3-ethyl-5-fluoro-4-[methyl-(1-methyl-1H-imidazo[4,5-c]pyridin-6-yl)-amino]-benzyl}-methane sulfonamide,
N-{3-Fluoro-4-[methyl-(1-methyl-1H-imidazo[4,5-c]pyridin-6-yl)-amino]-benzyl}-N-methyl-methanesulfonamide,
N-{3-Fluoro-4-[methyl-(1-methyl-1H-imidazo[4,5-c]pyridin-6-yl)-amino]-benzyl}-N-methoxymethyl-methanesulfonamide,
N-Cyclopropylmethyl-N-{3-fluoro-4-[methyl-(1-methyl-1H-imidazo[4,5-c]pyridin-6-yl)-amino]-benzyl}-methane sulfonamide,
3-{3-Difluoromethoxy-4-[methyl-(1-methyl-1H-imidazo[4,5-c]pyridin-6-yl)-amino]-phenyl}-azetidine-1-carboxylic acid tert-butyl ester,
[2-Difluoromethoxy-4-(1-methanesulfonyl-azetidin-3-yl)-phenyl]-methyl-(1-methyl-1H-imidazo[4,5-c]pyridin-6-yl)-amine,
{2-Difluoromethoxy-4-[1-(2,2,2-trifluoro-ethyl)-azetidin-3-yl]-phenyl}-methyl-(1-methyl-1H-imidazo[4,5-c]pyridin-6-yl)-amine,
{4-[1-(2,2-Difluoro-ethyl)-azetidin-3-yl]-2-difluoromethoxy-phenyl}-methyl-(1-methyl-1H-imidazo[4,5-c]pyridin-6-yl)-amine,
Azetidin-1-yl-(3-{3-difluoromethoxy-4-[methyl-(1-methyl-1H-imidazo[4,5-c]pyridin-6-yl)-amino]-phenyl}-azetidin-1-yl)-methanone,
(3-{3-Difluoromethoxy-4-[methyl-(1-methyl-1H-imidazo[4,5-c]pyridin-6-yl)-amino]-phenyl}-azetidin-1-yl)-piperidin-1-yl-methanone,
(3-{3-Difluoromethoxy-4-[methyl-(1-methyl-1H-imidazo[4,5-c]pyridin-6-yl)-amino]-phenyl}-azetidin-1-yl)-morpholin-4-yl-methanone,
3-{3-Difluoromethoxy-4-[methyl-(1-methyl-1H-imidazo[4,5-c]pyridin-6-yl)-amino]-phenyl}-azetidine-1-sulfonic acid dimethylamide,
{4-[1-(Difluoro-methanesulfonyl)-azetidin-3-yl]-2-difluoromethoxy-phenyl}-methyl-(1-methyl-1H-imidazo[4,5-c]pyridin-6-yl)-amine,
{2-Difluoromethoxy-4-[1-(morpholine-4-sulfonyl)-azetidin-3-yl]-phenyl}-methyl-(1-methyl-1H-imidazo[4,5-c]pyridin-6-yl)-amine,
{2-Difluoromethoxy-4-[1-(piperidine-1-sulfonyl)-azetidin-3-yl]-phenyl}-methyl-(1-methyl-1H-imidazo[4,5-c]pyridin-6-yl)-amine,
{2-Difluoromethoxy-4-[1-(propane-2-sulfonyl)-azetidin-3-yl]-phenyl}-methyl-(1-methyl-1H-imidazo[4,5-c]pyridin-6-yl)-amine,
2-(3-{3-Difluoromethoxy-4-[methyl-(1-methyl-1H-imidazo[4,5-c]pyridin-6-yl)-amino]-phenyl}-azetidin-1-yl)-acetamide,
Dimethyl amino-N-{3-Ethyl-4-[methyl-(1-methyl-1H-imidazo[4,5-c]pyridin-6-yl)-amino]-benzyl}-sulfonamide,
Methyl amino-N-{3-Ethyl-4-[methyl-(1-methyl-1H-imidazo[4,5-c]pyridin-6-yl)-amino]-benzyl}-sulfonamide,
4,4-Difluoro-cyclohexanecarboxylic acid 3-ethyl-4-[methyl-(1-methyl-1H-imidazo[4,5-c]pyridin-6-yl)-amino]-benzylamide,
[4-(3,3-Difluoro-azetidin-1-ylmethyl)-2-ethyl-phenyl]-methyl-(1-methyl-1H-imidazo[4,5-c]pyridin-6-yl)-amine,
{4-[(2,2-Difluoro-ethylamino)-methyl]-2-ethyl-phenyl}-methyl-(1-methyl-1H-imidazo[4,5-c]pyridin-6-yl)-amine,
{2-Ethyl-4-[(2,2,2-trifluoro-ethylamino)-methyl]-phenyl}-methyl-(1-methyl-1H-imidazo[4,5-c]pyridin-6-yl)-amine,
1-{3-Ethyl-4-[methyl-(1-methyl-1H-imidazo[4,5-c]pyridin-6-yl)-amino]-benzyl}-azetidine-3-carbonitrile,
[4-(3,3-Dimethyl-azetidin-1-ylmethyl)-2-ethyl-phenyl]-methyl-(1-methyl-1H-imidazo[4,5-c]pyridin-6-yl)-amine,
(4-Ethyl-1′-ethanesulfonyl-1′,2′,3′,4′,5′,6′-hexahydro-[2,4]bipyridinyl-5-yl)-methyl-(1-methyl-1H-imidazo[4,5-c]pyridin-6-yl)-amine,
{4-Ethyl-5-[methyl-(1-methyl-1H-imidazo[4,5-c]pyridin-6-yl)-amino]-3′,4′,5′,6′-tetrahydro-2′H-[2,4]bipyridinyl-1′-yl}-acetonitrile,
1-{3-Ethyl-4-[methyl-(1-methyl-1H-imidazo[4,5-c]pyridin-6-yl)-amino]-phenyl}-cyclopropanecarbonitrile,
1-(3,3-Dimethyl-azetidin-1-yl)-2-{3-ethyl-4-[methyl-(1-methyl-1H-imidazo[4,5-c]pyridin-6-yl)-amino]-phenyl}-ethanone,
N-(2-Cyano-1-{3-ethyl-4-[methyl-(1-methyl-1H-imidazo[4,5-c]pyridin-6-yl)-amino]-phenyl}-ethyl)-methanesulfonamide,
N-Cyanomethyl-N-{3-ethyl-4-[methyl-(1-methyl-1H-imidazo[4,5-c]pyridin-6-yl)-amino]-benzyl}-methanesulfonamide,
(3-{3-Ethyl-4-[methyl-(1-methyl-1H-imidazo[4,5-c]pyridin-6-yl)-amino]-phenyl}-oxetan-3-yl)-acetonitrile,
{3-Ethyl-4-[methyl-(1-methyl-1H-imidazo[4,5-c]pyridin-6-yl)-amino]-phenyl}-(4-methane sulfonyl-piperazin-1-yl)-acetonitrile,
1-(Cyano-{3-ethyl-4-[methyl-(1-methyl-1H-imidazo[4,5-c]pyridin-6-yl)-amino]-phenyl}-methyl)-azetidine-3-carbonitrile,
N-[2-(3-{3-Ethyl-4-[methyl-(1-methyl-1H-imidazo[4,5-c]pyridin-6-yl)-amino]-phenyl}-oxetan-3-yl)-ethyl]-methanesulfonamide,
{3-Ethyl-4-[methyl-(1-methyl-1H-imidazo[4,5-c]pyridin-6-yl)-amino]-phenyl}-morpholin-4-yl-acetonitrile,
Azetidin-1-yl-{3-ethyl-4-[methyl-(1-methyl-1H-imidazo[4,5-c]pyridin-6-yl)-amino]-phenyl}-acetonitrile,
(4,4-Difluoro-piperidin-1-yl)-{3-ethyl-4-[methyl-(1-methyl-1H-imidazo[4,5-c]pyridin-6-yl)-amino]-phenyl}-acetonitrile,
(4-Acetyl-piperazin-1-yl)-{3-ethyl-4-[methyl-(1-methyl-1H-imidazo[4,5-c]pyridin-6-yl)-amino]-phenyl}-acetonitrile,
N-[2-(3-{3-Ethyl-4-[methyl-(1-methyl-1H-imidazo[4,5-c]pyridin-6-yl)-amino]-phenyl}-oxetan-3-yl)-ethyl]-C,C-difluoro-methanesulfonamide,
Difluoro-methanesulfonic acid 3-{3-ethyl-4-[methyl-(1-methyl-1H-imidazo[4,5-c]pyridin-6-yl)-amino]-phenyl}-pyrrolidin-3-ylmethyl ester,
1-{4-Ethyl-5-[methyl-(1-methyl-1-imidazo[4,5-c]pyridin-6-yl)-amino]-pyridin-2-yl}-azetidine-3-carboxylic acid methyl ester,
1-{4-Ethyl-5-[methyl-(1-methyl-1-imidazo[4,5-c]pyridin-6-yl)-amino]-pyridin-2-yl}-azetidine-3-carboxylic acid methylamide,
1-{4-Ethyl-5-[methyl-(1-methyl-1-imidazo[4,5-c]pyridin-6-yl)-amino]-pyridin-2-yl}-azetidine-3-carboxylic acid cyanomethyl-amide,
1-{4-Ethyl-5-[methyl-(1-methyl-1-imidazo[4,5-c]pyridin-6-yl)-amino]-pyridin-2-yl}-azetidine-3-carbonitrile,
3,5-Difluoro-4-[methyl-(1-methyl-1H-imidazo[4,5-c]pyridin-6-yl)-amino]-benzonitrile,
N-{3,5-Difluoro-4-[methyl-(1-methyl-1H-imidazo[4,5-c]pyridin-6-yl)-amino]-benzyl}-methanesulfonamide,
{3,5-Difluoro-4-[methyl-(1-methyl-1H-imidazo[4,5-c]pyridin-6-yl)-amino]-phenyl}-acetonitrile,
{3-Ethyl-4-[methyl-(1-methyl-1H-imidazo[4,5-c]pyridin-6-yl)-amino]-phenyl}-acetonitrile,
{4-Ethyl-5-[methyl-(1-methyl-1-imidazo[4,5-c]pyridin-6-yl)-amino]-pyridin-2-yl}-acetonitrile,
3-{3,5-Difluoro-4-[methyl-(1-methyl-1H-imidazo[4,5-c]pyridin-6-yl)-amino]-phenyl}-azetidine-1-carboxylic acid tert-butyl ester,
[2,6-Difluoro-4-(1-methanesulfonyl-azetidin-3-yl)-phenyl]-methyl-(1-methyl-1H-imidazo[4,5-c]pyridin-6-yl)-amine,
{3-Ethyl-4-[methyl-(1-methyl-1H-imidazo[4,5-c]pyridin-6-yl)-amino]-phenyl}-(3-oxo-piperazin-1-yl)-acetonitrile,
[6-(3,3-Difluoro-pyrrolidin-1-yl)-4-ethyl-pyridin-3-yl]-methyl-(1-methyl-1H-imidazo[4,5-c]pyridin-6-yl)-amine,
1-{4-Ethyl-5-[methyl-(1-methyl-1-imidazo[4,5-c]pyridin-6-yl)-amino]-pyridin-2-yl}-azetidine-3-carboxylic acid ethylamide,
[6-(3,3-Difluoro-azetidin-1-yl)-4-ethyl-pyridin-3-yl]-methyl-(1-methyl-1H-imidazo[4,5-c]pyridin-6-yl)-amine,
[4-Ethyl-6-((R)-3-fluoro-pyrrolidin-1-yl)-pyridin-3-yl]-methyl-(1-methyl-1H-imidazo[4,5-c]pyridin-6-yl)-amine,
[4-Ethyl-6-((S)-3-fluoro-pyrrolidin-1-yl)-pyridin-3-yl]-methyl-(1-methyl-1H-imidazo[4,5-c]pyridin-6-yl)-amine,
4′-Ethyl-5′-[methyl-(1-methyl-1H-imidazo[4,5-c]pyridin-6-yl)-amino]-3,4,5,6-tetrahydro-2H-[1,2]bipyridinyl-3-carbonitrile,
[4-Ethyl-6-((S)-3-methyl-pyrrolidin-1-yl)-pyridin-3-yl]-methyl-(1-methyl-1H-imidazo[4,5-c]pyridin-6-yl)-amine,
1-{4-Ethyl-5-[methyl-(1-methyl-1H-imidazo[4,5-c]pyridin-6-yl)-amino]-pyridin-2-yl}-pyrrolidine-3-carbonitrile,
[2-Ethyl-4-(2-oxa-6-aza-spiro[3.3]hept-6-ylmethyl)-phenyl]-methyl-(1-methyl-1H-imidazo[4,5-c]pyridin-6-yl)-amine,
[2-Ethyl-4-(3-methanesulfonyl-pyrrolidin-1-ylmethyl)-phenyl]-methyl-(1-methyl-1H-imidazo[4,5-c]pyridin-6-yl)-amine,
4-{3-Ethyl-4-[methyl-(1-methyl-1H-imidazo[4,5-c]pyridin-6-yl)-amino]-benzyl}-piperazin-2-one,
[4-(4,4-Difluoro-piperidin-1-ylmethyl)-2-ethyl-phenyl]-methyl-(1-methyl-1H-imidazo[4,5-c]pyridin-6-yl)-amine,
N-{3-Ethyl-4-[methyl-(1-methyl-1H-imidazo[4,5-c]pyridin-6-yl)-amino]-benzyl}-amino sulfonamide,
3,3-Difluoro-azetidine-1-sulfonic acid 3-ethyl-4-[methyl-(1-methyl-1H-imidazo[4,5-c]pyridin-6-yl)-amino]-benzylamide,
3,3-Difluoro-pyrrolidine-1-sulfonic acid 3-ethyl-4-[methyl-(1-methyl-1H-imidazo[4,5-c]pyridin-6-yl)-amino]-benzylamide,
Morpholine-4-sulfonic acid 3-ethyl-4-[methyl-(1-methyl-1H-imidazo[4,5-c]pyridin-6-yl)-amino]-benzylamide,
(2-Difluoromethoxy-4-methyl-phenyl)-methyl-(1-methyl-1H-imidazo[4,5-c]pyridin-6-yl)-amine,
3-Difluoromethoxy-4-[methyl-(1-methyl-1H-imidazo[4,5-c]pyridin-6-yl)-amino]-benzonitrile,
N-{3-Difluoromethoxy-4-[methyl-(1-methyl-1H-imidazo[4,5-c]pyridin-6-yl)-amino]-benzyl}-methanesulfonamide,
N-{3-Difluoromethoxy-4-[methyl-(1-methyl-1H-imidazo[4,5-c]pyridin-6-yl)-amino]-benzyl}-N-methyl-methanesulfonamide,
(1-Difluoromethyl-1H-imidazo[4,5-c]pyridin-6-yl)-(2-ethyl-phenyl)-methyl-amine,
(6-Cyclohexyl-4-ethyl-pyridin-3-yl)-methyl-(1-methyl-1H-imidazo[4,5-c]pyridin-6-yl)-amine,
(6-Cyclopentyl-4-ethyl-pyridin-3-yl)-methyl-(1-methyl-1H-imidazo[4,5-c]pyridin-6-yl)-amine,
(6-Cyclobutyl-4-ethyl-pyridin-3-yl)-methyl-(1-methyl-1H-imidazo[4,5-c]pyridin-6-yl)-amine,
1-(Cyano-{3-ethyl-4-[methyl-(1-methyl-1H-imidazo[4,5-c]pyridin-6-yl)-amino]-phenyl}-methyl)-piperidine-4-carbonitrile,
[1-(Cyano-{3-ethyl-4-[methyl-(1-methyl-1H-imidazo[4,5-c]pyridin-6-yl)-amino]-phenyl}-methyl)-azetidin-3-yl]-carbamic acid tert-butyl ester,
{3-Ethyl-4-[methyl-(1-methyl-1H-imidazo[4,5-c]pyridin-6-yl)-amino]-phenyl}-(4-hydroxymethyl-piperidin-1-yl)-acetonitrile,
{3-Ethyl-4-[methyl-(1-methyl-1H-imidazo[4,5-c]pyridin-6-yl)-amino]-phenyl}-((R)-3-fluoro-pyrrolidin-1-yl)-acetonitrile,
{3-Ethyl-4-[methyl-(1-methyl-1H-imidazo[4,5-c]pyridin-6-yl)-amino]-phenyl}-((R)-2-methoxymethyl-pyrrolidin-1-yl)-acetonitrile,
(3,3-Difluoro-azetidin-1-yl)-{3-ethyl-4-[methyl-(1-methyl-1H-imidazo[4,5-c]pyridin-6-yl)-amino]-phenyl}-acetonitrile,
(3,3-Difluoro-pyrrolidin-1-yl)-{3-ethyl-4-[methyl-(1-methyl-1H-imidazo[4,5-c]pyridin-6-yl)-amino]-phenyl}-acetonitrile,
[6-(3-Amino-azetidin-1-yl)-4-ethyl-pyridin-3-yl]-methyl-(1-methyl-1H-imidazo[4,5-c]pyridin-6-yl)-amine,
N-(1-{4-Ethyl-5-[methyl-(1-methyl-1-imidazo[4,5-c]pyridin-6-yl)-amino]-pyridin-2-yl}-azetidin-3-yl)-methanesulfonamide,
{4-Ethyl-5-[methyl-(1-methyl-1H-imidazo[4,5-c]pyridin-6-yl)-amino]-pyridin-2-yl}-morpholin-4-yl-acetonitrile,
1-(Cyano-{3-ethyl-4-[methyl-(1-methyl-1H-imidazo[4,5-c]pyridin-6-yl)-amino]-phenyl}-methyl)-pyrrolidine-3-carbonitrile,
(3,3-Dimethyl-azetidin-1-yl)-{3-ethyl-4-[methyl-(1-methyl-1H-imidazo[4,5-c]pyridin-6-yl)-amino]-phenyl}-acetonitrile,
{3-Ethyl-4-[methyl-(1-methyl-1H-imidazo[4,5-c]pyridin-6-yl)-amino]-phenyl}-(4-hydroxy-piperidin-1-yl)-acetonitrile,
{3-Ethyl-4-[methyl-(1-methyl-1H-imidazo[4,5-c]pyridin-6-yl)-amino]-phenyl}-((R)-3-methoxy-pyrrolidin-1-yl)-acetonitrile,
{3-Ethyl-4-[methyl-(1-methyl-1H-imidazo[4,5-c]pyridin-6-yl)-amino]-phenyl}-(4-methyl-piperazin-1-yl)-acetonitrile,
{3-Ethyl-4-[methyl-(1-methyl-1H-imidazo[4,5-c]pyridin-6-yl)-amino]-phenyl}-(3-hydroxy-azetidin-1-yl)-acetonitrile,
1-(Cyano-{3-ethyl-4-[methyl-(1-methyl-1H-imidazo[4,5-c]pyridin-6-yl)-amino]-phenyl}-methyl)-piperidine-4-carboxylic acid amide,
{3-Ethyl-4-[methyl-(1-methyl-1H-imidazo[4,5-c]pyridin-6-yl)-amino]-phenyl}-(4-methanesulfonyl-piperidin-1-yl)-acetonitrile,
1-(Cyano-{3-ethyl-4-[methyl-(1-methyl-1H-imidazo[4,5-c]pyridin-6-yl)-amino]-phenyl}-methyl)-azetidine-3-carboxylic acid ethyl ester,
N-[1-(Cyano-{3-ethyl-4-[methyl-(1-methyl-1H-imidazo[4,5-c]pyridin-6-yl)-amino]-phenyl}-methyl)-azetidin-3-yl]-propionamide,
N-[1-(Cyano-{3-ethyl-4-[methyl-(1-methyl-1H-imidazo[4,5-c]pyridin-6-yl)-amino]-phenyl}-methyl)-azetidin-3-yl]-methanesulfonamide,
(4-Ethyl-pyrimidin-5-yl)-methyl-(1-methyl-1H-imidazo[4,5-c]pyridin-6-yl)-amine,
3-{6-Ethyl-5-[methyl-(1-methyl-1-imidazo[4,5-c]pyridin-6-yl)-amino]-pyrazin-2-yl}-azetidine-1-carboxylic acid tert-butyl ester,
3-{4-Ethyl-5-[methyl-(1-methyl-1H-imidazo[4,5-c]pyridin-6-yl)-amino]-pyridin-2-yl}-1-methanesulfonyl-azetidine-3-carbonitrile,
N-(2-ethylphenyl)-1-methyl-N-(2-(pyrrolidin-1-yl)ethyl)-1H-imidazo[4,5-c]pyridin-6-amine,
N-(2-ethylphenyl)-1-methyl-N-(2-morpholinoethyl)-1H-imidazo[4,5-c]pyridin-6-amine,
N-(2-ethylphenyl)-1-methyl-N-(pyridin-2-ylmethyl)-1H-imidazo[4,5-c]pyridin-6-amine,
N-(2-ethylphenyl)-1-methyl-N-(pyridin-3-ylmethyl)-1H-imidazo[4,5-c]pyridin-6-amine,
N-(2,2-difluoroethyl)-N-(2-ethylphenyl)-1-methyl-1H-imidazo[4,5-c]pyridin-6-amine, and
3-((2-ethylphenyl)(1-methyl-1H-imidazo[4,5-c]pyridin-6-yl)amino)propanenitrile.

In one embodiment, a compound of the invention is 3-(4-(3-ethyl-4-(methyl(1-methyl-1H-imidazo[4,5-c]pyridin-6-yl)amino)phenyl)-5,6-dihydropyridin-1 (2H)-yl)-3-oxopropanenitrile.

In one embodiment, a compound of the invention is not 3-(4-(3-ethyl-4-(methyl(1-methyl-1H-imidazo[4,5-c]pyridin-6-yl)amino)phenyl)-5,6-dihydropyridin-1 (2H)-yl)-3-oxopropanenitrile.

In one embodiment, a compound of the invention is [4-ethyl-6-(1-methanesulfonyl-azetidin-3-yl)-pyridin-3-yl]-methyl-(1-methyl-1H-imidazo[4,5-c]pyridin-6-yl)-amine.

In one embodiment, a compound of the invention is not [4-ethyl-6-(1-methanesulfonyl-azetidin-3-yl)-pyridin-3-yl]-methyl-(1-methyl-1H-imidazo[4,5-c]pyridin-6-yl)-amine.

In one embodiment a compound of the invention is not an isotopic variant.

In one aspect a compound of the invention according to any one of the embodiments herein described is present as the free base.

In one aspect a compound of the invention according to any one of the embodiments herein described is a pharmaceutically acceptable salt.

In one aspect a compound of the invention according to any one of the embodiments herein described is a solvate of the compound.

In one aspect a compound of the invention according to any one of the embodiments herein described is a solvate of a pharmaceutically acceptable salt of a compound.

While specified groups for each embodiment have generally been listed above separately, a compound of the invention includes one in which several or each embodiment in the above Formula, as well as other formulae presented herein, is selected from one or more of particular members or groups designated respectively, for each variable. Therefore, this invention is intended to include all combinations of such embodiments within its scope.

While specified groups for each embodiment have generally been listed above separately, a compound of the invention may be one for which one or more variables (for example, R groups) is selected from one or more embodiments according to any of the Formula (e) listed above. Therefore, the present invention is intended to include all combinations of variables from any of the disclosed embodiments within its scope.

Alternatively, the exclusion of one or more of the specified variables from a group or an embodiment, or combinations thereof is also contemplated by the present invention.

In certain aspects, the present invention provides prodrugs and derivatives of the compounds according to the formulae above. Prodrugs are derivatives of the compounds of the invention, which have metabolically cleavable groups and become by solvolysis or under physiological conditions the compounds of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (see, Bundgard, H. Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Particularly useful are the C₁ to C₈ alkyl, C₂-C₈ alkenyl, aryl, C₇-C₁₂ substituted aryl, and C₇-C₁₂ arylalkyl esters of the compounds of the invention.

The compounds of the invention are novel inhibitors of JAK. In particular, the compounds are potert inhibitors of JAK1 and/or JAK2; however they may inhibit TYK2 and JAK3 with a lower potency.

### Clauses

1. A compound according to Formula I:

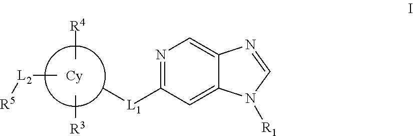

wherein
R¹ is Me, Et, or cyclopropyl, each of which is optionally substituted with one or more halo;
L₁ is —NR²—; —O—, or —CH₂—;
Cy is phenyl, or 5-9 membered heteroaryl comprising 1 to 4 heteroatoms independently selected from N, O, and S;
R² is
   C₁₋₄ alkyl optionally substituted with one or more groups independently selected from
      C₃₋₇ cycloalkyl,
      halo,
      CN,
      NR^{15a}R^{15b} wherein each R^{15a} and R^{15b} is independently selected from C₁₋₄ alkyl,
      4-6 membered heterocycloalkyl containing 1 to 2 heteroatoms independently selected from N, O, and S, and
      C₁₋₄ alkoxy, or
   C₃₋₇ cycloalkyl;
R³ is
   —H,
   halo,
   cyclopropyl,
   C₁₋₄ alkyl optionally substituted with one or more halo, or
   C₁₋₄ alkoxy optionally substituted with one or more halo;
R⁴ is H, or halo;
L₂ is
   absent or is
   —W—,
   —C₁₋₂ alkylene- (wherein the alkylene is optionally substituted with one CN), or
   —C₁₋₂ alkylene-W— (wherein the alkylene is optionally substituted with one CN), or
   —CH═CH—;
W is —C(═O)—, —C(═O)O—, —C(═O)NR⁶—, —NR⁶C(═O)—, —NR⁶C(═O)O—, —NR⁶C(═O)NH—, —S—, —SO₂—, —SO₂NR⁶—, —NHSO₂NR⁶—, —NR⁶SO₂—, —O—, or NR⁶;
R⁵ is:
   H,
   CN,
   C₁₋₆ alkyl optionally substituted with one or more independently selected R⁷ groups,
   C₃₋₇ cycloalkyl, optionally substituted with one or more groups independently selected from R¹⁰,
   4-7 membered heterocycloalkyl comprising 1 or 2 heteroatoms independently selected from N, O, and S, optionally substituted with one or more groups independently selected from R¹⁰,
   4-7 membered heterocycloalkenyl comprising 1 double bond, and comprising 1 or 2 heteroatoms independently selected from N, O, and S, optionally substituted with one or more groups independently selected from R¹⁰,
   C₆₋₁₀ aryl optionally substituted with one or more groups independently selected from R¹¹, or
   5-6 membered heteroaryl comprising 1 to 4 heteroatoms independently selected from N, O, and S, optionally substituted with one or more groups independently selected from R¹¹;
R⁶ is H, or C₁₋₄ alkyl optionally substituted with CN, C₁₋₂ alkoxy, or C₃₋₆ cycloalkyl;
R⁷ is
   OH,
   CN,
   halo,
   C₁₋₄ alkoxy,
   4-7 membered heterocycloalkyl comprising 1 or 2 heteroatoms independently selected from N, O, and S, optionally substituted with one or more groups independently selected from halo, C₁₋₄ alkyl and oxo,
   NR^{8a}R^{8b},
   5-6 membered heteroaryl comprising 1 to 4 heteroatoms independently selected from N, O, and S, optionally substituted with one or more independently selected C₁₋₄ alkyl, CN, halo, and C₁₋₄ alkoxy,
   phenyl optionally substituted with one or more groups independently selected from C₁₋₄ alkyl, CN, halo, and C₁₋₄ alkoxy,
   C₃₋₇ cycloalkyl, or
   —C(═O)NR^{9a}R^{9b},
   —OSO₂C₁₋₄ alkyl (which alkyl is optionally substituted with one or more halo), or
   —NR^{9c}SO₂C₁₋₄ alkyl (which alkyl is optionally substituted with one or more halo);
each R^{8a}, and R^{8b} is independently selected from H, and C₁₋₄ alkyl;
each R^{9a}, R^{9b} and R^{9c} is independently selected from H, and C₁₋₄ alkyl;
each R¹⁰ is independently selected from oxo or R¹¹;
each R¹¹ is halo, —CN or L₃-R¹²;
L₃ is absent or is —C(═O)—, C(═O)O—, —O—, SO₂—, —C(═O)NR^{13a}, —NR^{13b}C(═O), or NR^{13c};
each R¹² is
   —H,
   C₁₋₄ alkyl optionally substituted with one or more independently selected
      halo,
      OH,
      CN,
      C₁₋₄ alkoxy,
      NHC(═O)O—C₁₋₄ alkyl
      —C(═O)NR^{14a}R^{14b},
      —NR^{14c}R^{14d}
      —C(═O)C₁₋₄ alkyl
      —C(═O)O—C₁₋₄ alkyl
      phenyl optionally substituted with halo, C₁₋₄ alkyl, C₁₋₄ alkoxy, and
      4-7 membered heterocycloalkyl comprising 1 or 2 heteroatoms independently selected from N, O, and S, optionally substituted with one or more C₁₋₄ alkyl,
   4-7 membered heterocycloalkyl comprising 1 or 2 heteroatoms independently selected from N, O, and S, optionally substituted with one or more independently selected C₁₋₄ alkyl, oxo and CN,
   5-6 membered heteroaryl comprising 1 to 4 heteroatoms independently selected from N, O, and S, optionally substituted with one or more independently selected C₁₋₄ alkyl, or
   C₃₋₇ cycloalkyl optionally substituted with one or more independently selected OH, halo, C₁₋₄ alkyl, and CN;
each R^{13a}, R^{13b}, R^{13c}, R^{14a}, R^{14b}, R^{14c}, and R^{14d}, is independently selected from H, and C₁₋₄ alkyl;
provided that R³, R⁴, and -L₂-R⁵ are not all simultaneously H when Cy is C₆ aryl, or 6-membered heteroaryl; or
a pharmaceutically acceptable salt, or a solvate, or a solvate of the pharmaceutically acceptable salts.
2. A compound according to Formula I:

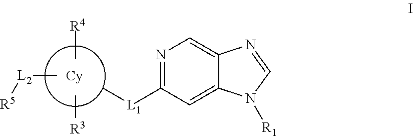

wherein
R¹ is Me, Et, or cyclopropyl, each of which is optionally substituted with one or more halo;
L₁ is —NR²—; —O—, or —CH₂—;
Cy is phenyl, or 5-9 membered heteroaryl comprising 1 or 2 heteroatoms independently selected from N, O, and S;
R² is
   C₁₋₂, alkyl optionally substituted with one or more groups independently selected from
      C₃₋₇ cycloalkyl, and
      halo, or
   C₃₋₇ cycloalkyl;
R³ is
   H,
   halo,
   cyclopropyl,
   C₁₋₄ alkyl optionally substituted with one or more halo, or
   C₁₋₄ alkoxy optionally substituted with one or more halo;
R⁴ is H, or halo;
L₂ is
   absent or is
   —W—,
   —C₁₋₂ alkylene-, or
   —C₁₋₂ alkylene-W—;
W is —C(═O)—, —C(═O)O—, —C(═O)NR⁶—, —NR⁶C(═O)—, —NR⁶C(═O)O—, —NR⁶C(═O)NH—, —S—, —SO₂—, —SO₂NR⁶—, —NR⁶SO₂—, —O—, or NR⁶;
R⁵ is:
   H,
   CN,
   C₁₋₆ alkyl optionally substituted with one or more independently selected R⁷ groups,
   C₃₋₇ cycloalkyl, optionally substituted with one or more groups independently selected from R¹⁰,
   4-7 membered heterocycloalkyl comprising 1 or 2 heteroatoms independently selected from N, O, and S, optionally substituted with one or more groups independently selected from R¹⁰,
   4-7 membered heterocycloalkenyl comprising 1 double bond, and comprising 1 or 2 heteroatoms independently selected from N, O, and S, optionally substituted with one or more groups independently selected from R¹⁰,
   C₆₋₁₀ aryl optionally substituted with one or more groups independently selected from R¹¹, or
   5-6 membered heteroaryl comprising 1 or 2 heteroatoms independently selected from N, O, and S, optionally substituted with one or more groups independently selected from R¹¹;
R⁶ is H, or C₁₋₄ alkyl;
R⁷ is
   OH,
   CN,
   halo,
   C₁₋₄ alkoxy,
   4-7 membered heterocycloalkyl comprising 1 or 2 heteroatoms independently selected from N, O, and S, optionally substituted with one or more groups independently selected from halo, C₁₋₄ alkyl and oxo,
   NR^{8a}R^{8b},
   5-6 membered heteroaryl comprising 1 or 2 heteroatoms independently selected from N, O, and S, optionally substituted with one or more independently selected C₁₋₄ alkyl, CN, halo, and C₁₋₄ alkoxy,
   phenyl optionally substituted with one or more groups independently selected from C₁₋₄ alkyl, CN, halo, and C₁₋₄ alkoxy,
   C₃₋₇ cycloalkyl, or
   —C(═O)NR^{9a}R^{9b};
each R^{8a}, and R^{8b} is independently selected from H, and C₁₋₄ alkyl;
each R^{9a}, and R^{9b} is independently selected from H, and C₁₋₄ alkyl;
each R¹⁰ is independently selected from oxo or R¹¹;
each R¹¹ is halo, —CN or L₃-R¹²;
L₃ is absent or is —C(═O)—, C(═O)O—, —O—, SO₂—, —C(═O)NR^{13a}, —NR^{13b}C(═O), or NR^{13c};
each R¹² is
   H,
   C₁₋₄ alkyl optionally substituted with one or more independently selected
      halo,
      OH,
      CN,
   C₁₋₄ alkoxy,
      NHC(═O)O—C₁₋₄ alkyl
      —C(═O)NR^{14a}R^{14b},
      —NR^{14c}R^{14d}
      —C(═O)C₁₋₄ alkyl
      —C(═O)O—C₁₋₄ alkyl
      phenyl optionally substituted with halo, C₁₋₄ alkyl, C₁₋₄ alkoxy, and
      4-7 membered heterocycloalkyl comprising 1 or 2 heteroatoms independently selected from N, O, and S, optionally substituted with one or more C₁₋₄ alkyl,
   4-7 membered heterocycloalkyl comprising 1 or 2 heteroatoms independently selected from N, O, and S, optionally substituted with one or more independently selected C₁₋₄ alkyl, oxo and CN,
   5-6 membered heteroaryl comprising 1 or 2 heteroatoms independently selected from N, O, and S, optionally substituted with one or more independently selected C₁₋₄ alkyl, or
   C₃₋₇ cycloalkyl optionally substituted with one or more independently selected OH, halo, C₁₋₄ alkyl, and CN;
each R^{13a}, R^{13b}, R^{13c}, R^{14a}, R^{14b}, R^{14c}, and R^{14d}, is independently selected from H, and C₁₋₄ alkyl;
provided that R³, R⁴, and -L₂-R⁵ are not all simultaneously H when Cy is C₆ aryl, or 6-membered heteroaryl; or
a pharmaceutically acceptable salt, or a solvate, or a solvate of the pharmaceutically acceptable salts.
3. A compound or pharmaceutically acceptable salt according to clause 1 or 2, wherein R¹ is Me or Et.
4. A compound or pharmaceutically acceptable salt thereof according to any one of clauses 1-3,
wherein Cy is 5-6 membered heteroaryl comprising 1 to 4 heteroatoms independently selected from N, O, and S.
5. A compound or pharmaceutically acceptable salt according to any one of clauses 1-4, wherein Cy is pyridyl.
6. A compound or pharmaceutically acceptable salt according to any one of clauses 1-3, wherein Cy is phenyl.
7. A compound or pharmaceutically acceptable salt according to any one of clauses 1-6, wherein R⁴ is F, or Cl.
8. A compound or pharmaceutically acceptable salt according to any one of clauses 1-6, wherein R⁴ is H.
9. A compound or pharmaceutically acceptable salt according to clause 1 or 2, wherein the compound or pharmaceutically acceptable salt is according to Formula IIa or IIb:

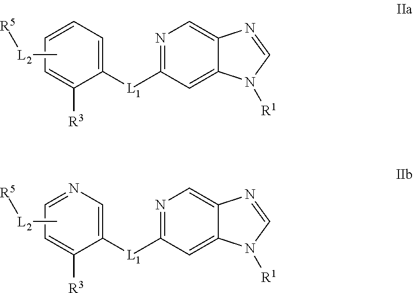

wherein R¹, L₁, R³, L₂, and R⁵ are as described in clause 1 or 2.
10. A compound or pharmaceutically acceptable salt according to clause 1 or 2, wherein the compound or pharmaceutically acceptable salt is according to Formula IIIa or MIII:

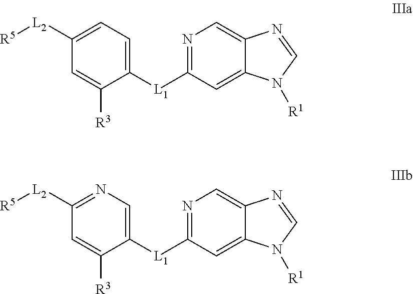

wherein L₁, R³, L₂, and R⁵ are as described in clause 1 or 2.
11. A compound or pharmaceutically acceptable salt according to any of clauses clause 1-10, wherein L₁ is —CH₂—
12. A compound or pharmaceutically acceptable salt according to any of clauses clause 1-10, wherein L₁ is —O—.
13. A compound or pharmaceutically acceptable salt according to any of clauses clause 1-10, wherein L₁ is —NR², and R² is as defined in clause 1.
14. A compound or pharmaceutically acceptable salt according to clause 13, wherein R² is —CH₃, —C₂H₅, —CH₂-cyclopropyl, or cyclopropyl.
15. A compound or pharmaceutically acceptable salt according to any one of clauses 1-14, wherein L₂ is absent.
16. A compound or pharmaceutically acceptable salt according to any one of clauses 1-14, wherein L₂ is —CH₂—.
17. A compound or pharmaceutically acceptable salt according to any one of clauses 1-14, wherein L₂ is —W—.
18. A compound or pharmaceutically acceptable salt according to any one of clauses 1-14, wherein L₂ is —CH₂—W—.
19. A compound or pharmaceutically acceptable salt according to clause 17 or 18, wherein W is —C(═O)—, —SO₂—, or —O—.
20. A compound or pharmaceutically acceptable salt according to clause 17 or 18, wherein W is —C(═O)NR⁶—, —NR⁶C(═O)O—, —NR⁶C(═O)NH—, —NR⁶SO₂—, or —NR⁶.
21. A compound or pharmaceutically acceptable salt according to clause 20, wherein R⁶ is H, Me or Et.
22. A compound or pharmaceutically acceptable salt according to clause 1 or 2, wherein the compound is according to Formula IVa-IVf:

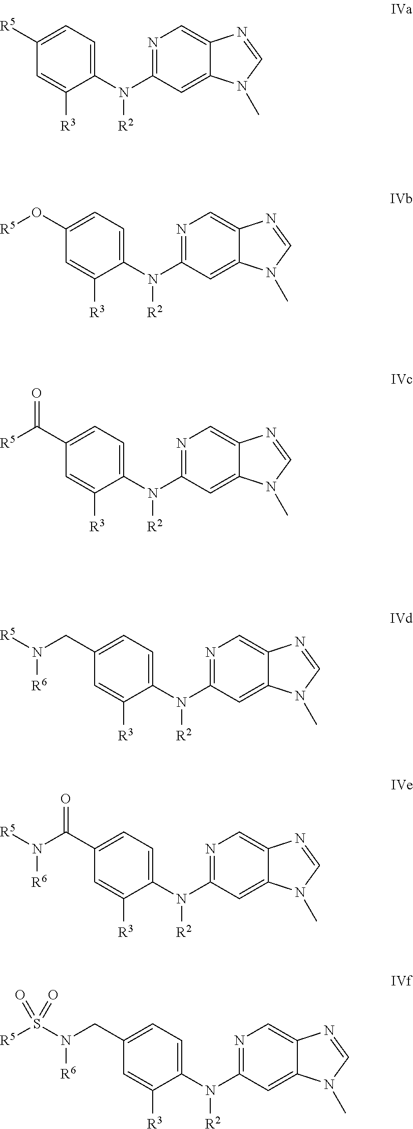

wherein R², R³, R⁶, and R⁵ are as described in clause 1 or 2.
23. A compound or pharmaceutically acceptable salt according to clause 1 or 2, wherein the compound is according to any one of Formulae IVg-IVl:

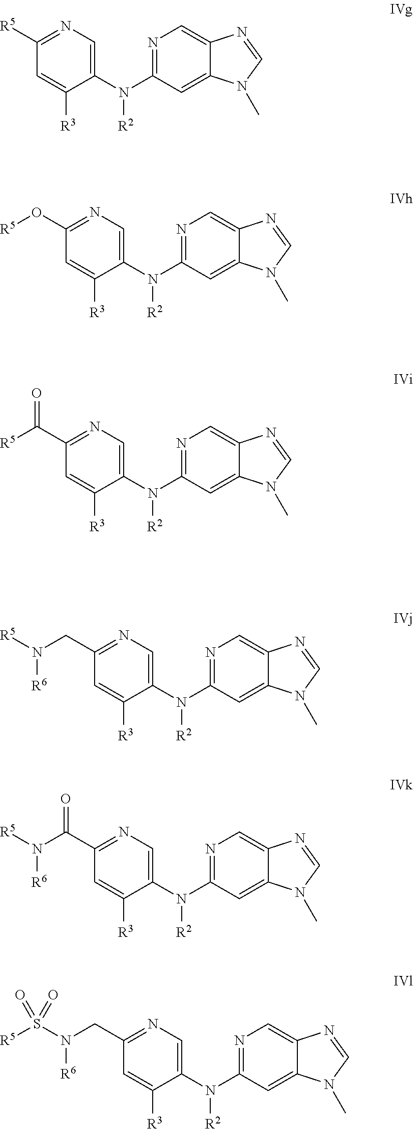

wherein R², R³, R⁶, and R⁵ are as described in any of the embodiments above.
24. A compound or pharmaceutically acceptable salt according to clause 22 or 23, wherein R³ is C₁₋₄ alkyl.
25. A compound or pharmaceutically acceptable salt according to clause 24, wherein R³ is —CH₃, or —C₂H₅.
26. A compound or pharmaceutically acceptable salt according to clause 22 or 23, wherein R² is C₁₋₄ alkyl.
27. A compound or pharmaceutically acceptable salt according to clause 26, wherein R² is —CH₃, or —C₂H₅.
28. A compound or pharmaceutically acceptable salt according to clause 22 or 23, wherein the compound is according to any one of Formulae VId, VIe, VIf, VIj, VIk, or VIl and R⁶ is H, Me or Et.
29. A compound or pharmaceutically acceptable salt according to any one of clauses 1-28, wherein R⁵ is C₁₋₆ alkyl.
30. A compound or pharmaceutically acceptable salt according to any one of clauses 1-28, wherein R⁵ is C₁₋₆ alkyl substituted with one or more independently selected R⁷ groups.
31. A compound or pharmaceutically acceptable salt according to clause 30, wherein each R⁷ is selected from —OH, —CN, F, —OMe, —OEt, tetrahydrofuranyl, N-pyrrolidinyl, —N-methylpiperidin-2-yl, —NMe₂, 4-cyano-pyridin-3-yl, 2-pyridinyl, 3-pyridinyl, 2,5-dimethyl-isooxazolyl, phenyl, 4-chlorophenyl, 4-methoxyphenyl, cyclopropyl, and —C(═O)NMe₂.
32. A compound or pharmaceutically acceptable salt according to clause 29, 30 or 31, wherein R⁵ is Me, Et, or n-Pr.
33. A compound or pharmaceutically acceptable salt according to any one of clauses 1-28, wherein R⁵ is C₃₋₇ cycloalkyl.
34. A compound or pharmaceutically acceptable salt according to any one of clauses 1-28, wherein R⁵ is C₃₋₇ cycloalkyl, substituted with one or more groups independently selected from R¹⁰.
35. A compound or pharmaceutically acceptable salt according to clause 33 or 34, wherein R⁵ is cyclopropyl.
36. A compound or pharmaceutically acceptable salt according to any one of clauses 1-28, wherein R⁵ is 4-7 membered heterocycloalkyl, comprising 1 or 2 heteroatoms independently selected from N, O, and S.
37. A compound or pharmaceutically acceptable salt according to any one of clauses 1-28, wherein R⁵ is 4-7 membered heterocycloalkyl comprising 1 or 2 heteroatoms independently selected from N, O, and S, substituted with one or more groups independently selected from R¹⁰.
38. A compound or pharmaceutically acceptable salt according to clause 36 or 37, wherein R⁵ is oxetanyl, azetidinyl, tetrahydrofuranyl, pyrrolidinyl, tetrahydropyranyl, piperidinyl, piperazinyl, morpholinyl or thiomorpholinyl.
39. A compound or pharmaceutically acceptable salt according to any one of clauses 1-28, wherein R⁵ is 4-7 membered heterocycloalkenyl comprising 1 double bond, and comprising 1 or 2 heteroatoms independently selected from N, O, and S.
40. A compound or pharmaceutically acceptable salt according to any one of clauses 1-28, wherein R⁵ is 4-7 membered heterocycloalkenyl comprising 1 double bond, and comprising 1 or 2 heteroatoms independently selected from N, O, and S, substituted with one or more groups independently selected from R¹⁰.
41. A compound or pharmaceutically acceptable salt according to clause 39 or 40, wherein R⁵ is dihydropyranyl, or tetrahydropyridinyl.
42. A compound or pharmaceutically acceptable salt according to any one of clauses 1-28, wherein R⁵ is C₆₋₁₀ aryl.
43. A compound or pharmaceutically acceptable salt according to any one of clauses 1-28, wherein R⁵ is C₆₋₁₀ aryl, substituted with one or more groups independently selected from R¹¹.
44. A compound or pharmaceutically acceptable salt according to clause 42 or 43, wherein R⁵ is phenyl.
45. A compound or pharmaceutically acceptable salt according to any one of clauses 1-28, wherein R⁵ is 5-6 membered heteroaryl comprising 1 to 4 heteroatoms independently selected from N, O, and S.
46. A compound or pharmaceutically acceptable salt according to any one of clauses 1-28, wherein R⁵ is 5-6 membered heteroaryl comprising 1 to 4 heteroatoms independently selected from N, O, and S, substituted with one or more groups independently selected from R¹¹.
47. A compound or pharmaceutically acceptable salt according to clause 45 or 46, wherein R⁵ is pyrrolyl, furanyl, thiophenyl, imidazolyl, triazolyl, tetrazolyl, oxazolyl, thiazolyl, oxadiazolyl, thiadiazolyl, pyridinyl, or pyrazinyl.
48. A compound or pharmaceutically acceptable salt according to clauses 34, 37 or 40, wherein R¹⁰ is oxo.
49. A compound or pharmaceutically acceptable salt according to clauses 34, 37 or 40, wherein R¹⁰ is R¹¹, and R¹¹ is as defined in clause 1 or 2.
50. A compound or pharmaceutically acceptable salt according to clauses 42, 46, or 49, wherein R¹¹ is CN, F, or Cl.
51. A compound or pharmaceutically acceptable salt according to clauses 42, 46, or 49, wherein R¹¹ is L₃-R′², and L₃ and R¹² are as defined in clause 1 or 2.
52. A compound or pharmaceutically acceptable salt according to any one of clauses 1, 2, 9, 10, 22 or 23, wherein R⁵ is according to Formula V:

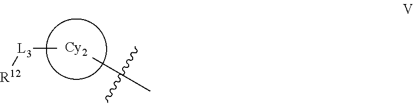

wherein Cyₜ is selected from
   C₃₋₇ cycloalkyl,
   4-7 membered heterocycloalkyl comprising 1 or 2 heteroatoms independently selected from N, O, and S, 4-7 membered heterocycloalkenyl comprising 1 double bond, and comprising 1 or 2 heteroatoms independently selected from N, O, and S,
   C₆₋₁₀ aryl, and
   5-6 membered heteroaryl comprising 1 to 4 heteroatoms independently selected from N, O, and S;
L₃ and R¹² are as described above.
53. A compound or pharmaceutically acceptable salt according to clause 1 or 2, wherein the compound is according to Formula VI:

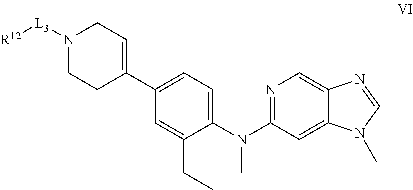

wherein L₃ and R¹² are as described in clause for 2.
54. A compound or pharmaceutically acceptable salt according to clause 1 or 2, wherein the compound is according to Formula VII:

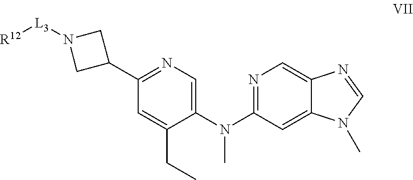

wherein L₃ and R¹² are as described in clause 1 or 2.
55. A compound or pharmaceutically acceptable salt according to any one of clauses 51-54, wherein
L₃ is absent.
56. A compound or pharmaceutically acceptable salt according to any one of clauses 51-54, wherein L₃ is —C(═O)—, —C(═O)O—, —O—, or SO₂—.
57. A compound or pharmaceutically acceptable salt according to any one of clauses 51-54, wherein L₃ is —C(═O)NR^{13a}, —NR^{13b}C(═O), or —NR^{13c}.
58. A compound or pharmaceutically acceptable salt according to clause 57, wherein each of R^{13a}, R^{13b}, and R^{13c} is independently selected from H, Me or Et.
59. A compound or pharmaceutically acceptable salt according to clauses any one of clauses 51-58, wherein R¹² is H.
60. A compound or pharmaceutically acceptable salt according to clauses any one of clauses 51-58, wherein R¹² is Me, Et, n-Pr, i-Pr, or t-Bu.
61. A compound or pharmaceutically acceptable salt according to clauses any one of clauses 51-58, wherein R¹² is —CH₂—CN, —CH₂—CH₂—CN, —CH₂—CH₂—OH—C(OH)H—CH₃, —C(OH)H—CF₃, —CHF₂, —CH₂—CF₃, —CH₂—CMe₂-OH, —CMeH—OMe, —CH₂—OH, —CMe₂-OH, —CH₂—OMe, —CH₂—C(═O)t-Bu, —CH₂—C(═O)NH₂, —CH₂-(1-methyloxetan-3-yl), benzyl, —CH₂-4-fluorophenyl, —CH₂-4-chlorophenyl, —CH₂-4-methylphenyl, —CH₂—CH₂(N-pyrrolidinyl), or —CH₂—CH₂—NMe₂.
62. A compound or pharmaceutically acceptable salt according to any one of clauses 51-58, wherein R¹² is piperidinyl, morpholinyl, oxetane, piperidinyl substituted with one Me, or CN.
63. A compound or pharmaceutically acceptable salt according to any one of clauses 51-58, wherein R¹² is thiadiazolyl.
64. A compound or pharmaceutically acceptable salt according to any one of clauses 51-58, wherein R¹² is cycloalkyl substituted with OH, CN, or Me.
65. The compound, or pharmaceutically acceptable salt thereof, according to clause 1 or 2 wherein the compound is selected from Table I
66. A pharmaceutical composition comprising a pharmaceutically acceptable carrier and a pharmaceutically effective amount of a compound according to any one of clauses 1-65.
67. The pharmaceutical composition according to clause 66 comprising a further therapeutic agent.
68. The compound or pharmaceutically acceptable salt thereof, according to any one of clauses 1-65, or the pharmaceutical composition according any one of clauses 66-67, for use in medicine.
69. A compound according to any one of clauses 1-65, or the pharmaceutical composition according any one of clauses 66-67, for use in the treatment, or prophylaxis of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.
70. A method for the treatment, or prophylaxis of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons, comprising administering an amount of compound according to any one of clauses 1-65, or the pharmaceutical composition according any one of clauses 66-67, sufficient to effect said treatment, or prophylaxis.
71. The method according to clause 70, wherein the compound according to any one of clauses 1-65, or the pharmaceutical composition according any one of clauses 66-67, is administered in combination with a further therapeutic agent.
72. The pharmaceutical composition according to clause 67, or the method according to clause 71, wherein the further therapeutic agent is an agent for the treatment, or prophylaxis of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.

### Pharmaceutical Compositions

When employed as a pharmaceutical, a compound of the invention is typically administered in the form of a pharmaceutical composition. Such compositions can be prepared in a manner well known in the pharmaceutical art and comprise at least one active compound. Generally, a compound of this invention is administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the invention can be administered by a variety of routes including oral, rectal, transdermal, subcutaneous, intra-articular, intravenous, intramuscular, and intranasal. Depending on the intended route of delivery, a compound of this invention is preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term ‘unit dosage forms’ refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions. In such compositions, a compound of the invention is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

A compound of the invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

A compound of the invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

A compound of the invention may be admixed as a dry powder with a dry gelatin binder in an approximate 1:2 weight ratio. A minor amount of magnesium stearate may be added as a lubricant. The mixture may be formed into 240-270 mg tablets (80-90 mg of active amide compound per tablet) in a tablet press.

### Formulation 2—Capsules

A compound of the invention may be admixed as a dry powder with a starch diluent in an approximate 1:1 weight ratio. The mixture may be filled into 250 mg capsules (125 mg of active amide compound per capsule).

### Formulation 3—Liquid

A compound of the invention (125 mg), may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color may be diluted with water and added with stilling. Sufficient water may then be added with stirring. Further sufficient water may be then added to produce a total volume of 5 mL.

### Formulation 4—Tablets

A compound of the invention may be admixed as a dry powder with a dry gelatin binder in an approximate 1:2 weight ratio. A minor amount of magnesium stearate may be added as a lubricant. The mixture may be formed into 450-900 mg tablets (150-300 mg of active amide compound) in a tablet press.

### Formulation 5—Injection

A compound of the invention may be dissolved or suspended in a buffered sterile saline injectable aqueous medium to a concentration of approximately 5 mg/mL.

### Formulation 6—Topical

Stearyl alcohol (250 g) and a white petrolatum (250 g) may be melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) may be added and the resulting mixture may be stirred until it congeals.

### Methods of Treatment

A compound of the invention may be used as a therapeutic agent for the treatment of conditions in mammals that are causally related or attributable to aberrant activity of JAK. In particular, conditions related to aberrant activity of JAK1 and/or JAK2. Accordingly, the compounds and pharmaceutical compositions of the invention find use as therapeutics for preventing and/or treating allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons in mammals including humans.

In one aspect, the present invention provides a compound of the invention, or a pharmaceutical composition comprising a compound of the invention for use as a medicament.

In another aspect, the present invention provides a compound of the invention, or a pharmaceutical composition comprising a compound of the invention for use in the manufacture of a medicament.

In yet another aspect, the present invention provides a method of treating a mammal having, or at risk of having a disease disclosed herein, said method comprising administering an effective condition-treating or condition-preventing amount of one or more of the pharmaceutical compositions or compound of the invention herein described. In a particular aspect, the present invention provides a method of treating a mammal having, or at risk of having allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.

In a method of treatment aspects, this invention provides methods of treatment and/or prophylaxis of a mammal susceptible to or afflicted with an allergic reaction, said method comprising administering an effective condition-treating or condition-preventing amount of one or more of the pharmaceutical compositions or compound of the invention herein described. In a specific embodiment, the allergic reaction is selected from allergic airway disease, sinusitis, eczema and hives, food allergies and allergies to insect venom.

In another aspect the present invention provides a compound of the invention for use in the treatment, and/or prophylaxis of an allergic reaction. In a specific embodiment, the allergic reaction is selected from allergic airway disease, sinusitis, eczema and hives, food allergies and allergies to insect venom.

In yet another aspect, the present invention provides the compound of the invention, or a pharmaceutical composition comprising the compound of the invention for use in the manufacture of a medicament for the treatment, or prophylaxis of an allergic reaction. In a specific embodiment, the allergic reaction is selected from allergic airway disease, sinusitis, eczema and hives, food allergies and allergies to insect venom.

In additional method of treatment aspects, this invention provides methods of treatment and/or prophylaxis of a mammal susceptible to or afflicted with an inflammatory condition. The methods comprise administering an effective condition-treating or condition-preventing amount of one or more of the pharmaceutical compositions or compound of the invention herein described. In a specific embodiment, the inflammatory condition is selected from rheumatoid arthritis, osteoarthritis, allergic airway disease (e.g. asthma) and inflammatory bowel diseases.

In another aspect the present invention provides a compound of the invention for use in the treatment, and/or prophylaxis of an inflammatory condition. In a specific embodiment, the inflammatory condition is selected from rheumatoid arthritis, osteoarthritis, allergic airway disease (e.g. asthma) and inflammatory bowel diseases.

In yet another aspect, the present invention provides the compound of the invention, or a pharmaceutical composition comprising the compound of the invention for use in the manufacture of a medicament for the treatment, and/or prophylaxis of an inflammatory condition. In a specific embodiment, the inflammatory condition is selected from rheumatoid arthritis, osteoarthritis, allergic airway disease (e.g. asthma) and inflammatory bowel diseases.

In additional method of treatment aspects, this invention provides methods of treatment and/or prophylaxis of a mammal susceptible to or afflicted with an autoimmune disease. The methods comprise administering an effective condition-treating or condition-preventing amount of one or more of the pharmaceutical compositions or compounds of the invention herein described. In a specific embodiment, the autoimmune disease is selected from COPD, asthma, systemic lupus erythematosis, type I diabetes mellitus and inflammatory bowel disease.

In another aspect the present invention provides a compound of the invention for use in the treatment, and/or prophylaxis of an autoimmune disease. In a specific embodiment, the autoimmune disease is selected from COPD, asthma, systemic lupus erythematosis, type I diabetes mellitus and inflammatory bowel disease. In a more specific embodiment, the autoimmune disease is systemic lupus erythematosis.

In yet another aspect, the present invention provides the compound of the invention, or a pharmaceutical composition comprising the compound of the invention for use in the manufacture of a medicament for the treatment, and/or prophylaxis of an autoimmune disease. In a specific embodiment, the autoimmune disease is selected from COPD, asthma, systemic lupus erythematosis, type I diabetes mellitus and inflammatory bowel disease.

In further method of treatment aspects, this invention provides methods of treatment and/or prophylaxis of a mammal susceptible to or afflicted with a proliferative disease, said methods comprising administering an effective condition-treating or condition-preventing amount of one or more of the pharmaceutical compositions or compound of the invention herein described. In a specific embodiment, the proliferative disease is selected from cancer (e.g. solid tumors such as uterine leiomyosarcoma or prostate cancer), leukemia (e.g. AML, ALL or CLL), multiple myeloma and psoriasis.

In another aspect the present invention provides a compound of the invention for use in the treatment, and/or prophylaxis of a proliferative disease. In a specific embodiment, the proliferative disease is selected from cancer (e.g. solid tumors such as uterine leiomyosarcoma or prostate cancer), leukemia (e.g. AML, ALL or CLL), multiple myeloma and psoriasis.

In yet another aspect, the present invention provides the compound of the invention, or a pharmaceutical composition comprising the compound of the invention for use in the manufacture of a medicament for the treatment, and/or prophylaxis of a proliferative disease. In a specific embodiment, the proliferative disease is selected from cancer (e.g. solid tumors such as uterine leiomyosarcoma or prostate cancer), leukemia (e.g. AML, ALL or CLL), multiple myeloma and psoriasis.

In further method of treatment aspects, this invention provides methods of treatment and/or prophylaxis of a mammal susceptible to or afflicted with transplantation rejection, said methods comprising administering an effective condition-treating or condition-preventing amount of one or more of the pharmaceutical compositions or compound of the invention herein described. In a specific embodiment, the transplantation rejection is organ transplant rejection.

In another aspect the present invention provides a compound of the invention for use in the treatment, and/or prophylaxis of transplantation rejection. In a specific embodiment, the transplantation rejection is organ transplant rejection.

In yet another aspect, the present invention provides the compound of the invention, or a pharmaceutical composition comprising the compound of the invention for use in the manufacture of a medicament for the treatment and/or prophylaxis of transplantation rejection. In a specific embodiment, the transplantation rejection is organ transplant rejection.

In a method of treatment aspect, this invention provides a method of treatment, and/or prophylaxis in a mammal susceptible to or afflicted with diseases involving impairment of cartilage turnover, which method comprises administering a therapeutically effective amount of a compound of the invention, or one or more of the pharmaceutical compositions herein described.

In another aspect the present invention provides a compound of the invention for use in the treatment, and/or prophylaxis of diseases involving impairment of cartilage turnover.

In yet another aspect, the present invention provides the compound of the invention, or a pharmaceutical composition comprising the compound of the invention for use in the manufacture of a medicament for the treatment, and/or prophylaxis of diseases involving impairment of cartilage turnover.

The present invention also provides a method of treatment and/or prophylaxis of congenital cartilage malformations, which method comprises administering an effective amount of one or more of the pharmaceutical compositions or compounds of the invention herein described.

In another aspect the present invention provides a compound of the invention for use in the treatment, and/or prophylaxis of congenital cartilage malformations.

In yet another aspect, the present invention provides the compound of the invention, or a pharmaceutical composition comprising the compound of the invention for use in the manufacture of a medicament for the treatment, and/or prophylaxis of congenital cartilage malformations.

In further method of treatment aspects, this invention provides methods of treatment and/or prophylaxis of a mammal susceptible to or afflicted with diseases associated with hypersecretion of IL6, said methods comprising administering an effective condition-treating or condition-preventing amount of one or more of the pharmaceutical compositions or compound of the invention herein described. In a specific embodiment, the disease associated with hypersecretion of IL6 is selected from Castleman's disease and mesangial proliferative glomerulonephritis.

In another aspect the present invention provides a compound of the invention for use in the treatment, and/or prophylaxis of diseases associated with hypersecretion of IL6. In a specific embodiment, the disease associated with hypersecretion of IL6 is selected from Castleman's disease and mesangial proliferative glomerulonephritis.

In yet another aspect, the present invention provides the compound of the invention, or a pharmaceutical composition comprising the compound of the invention for use in the manufacture of a medicament for the treatment, and/or prophylaxis of diseases associated with hypersecretion of IL6. In a specific embodiment, the disease associated with hypersecretion of IL6 is selected from Castleman's disease and mesangial proliferative glomerulonephritis.

In further method of treatment aspects, this invention provides methods of treatment and/or prophylaxis of a mammal susceptible to or afflicted with diseases associated with hypersecretion of interferons, said methods comprising administering an effective condition-treating or condition-preventing amount of one or more of the pharmaceutical compositions or compound of the invention herein described. In a specific embodiment, the disease associated with hypersecretion of interferons is selected from systemic and cutaneous lupus erythematosis, lupus nephritis, dermatomyositis, Sjogren's syndrome, psoriasis, and rheumatoid arthritis.

In another aspect the present invention provides a compound of the invention for use in the treatment, and/or prophylaxis of diseases associated with hypersecretion of interferons. In a specific embodiment, the disease associated with hypersecretion of interferons is selected from systemic and cutaneous lupus erythematosis, lupus nephritis, dermatomyositis, Sjogren's syndrome, psoriasis, and rheumatoid arthritis.

In yet another aspect, the present invention provides the compound of the invention, or a pharmaceutical composition comprising the compound of the invention for use in the manufacture of a medicament for the treatment, and/or prophylaxis of diseases associated with hypersecretion of interferons. In a specific embodiment, the disease associated with hypersecretion of interferons is selected from systemic and cutaneous lupus erythematosis, lupus nephritis, dermatomyositis, Sjogren's syndrome, psoriasis, and rheumatoid arthritis.

As a further aspect of the invention there is provided a compound of the invention for use as a pharmaceutical especially in the treatment and/or prophylaxis of the aforementioned conditions and diseases. Also provided herein is the use of the present compounds in the manufacture of a medicament for the treatment and/or prophylaxis of one of the aforementioned conditions and diseases.

A particular regimen of the present method comprises the administration to a subject suffering from a disease involving inflammation, of an effective amount of a compound of the invention for a period of time sufficient to reduce the level of inflammation in the subject, and preferably terminate the processes responsible for said inflammation. A special embodiment of the method comprises administering of an effective amount of a compound of the invention to a subject patient suffering from or susceptible to the development of rheumatoid arthritis, for a period of time sufficient to reduce or prevent, respectively, inflammation in the joints of said patient, and preferably terminate, the processes responsible for said inflammation.

A further particular regimen of the present method comprises the administration to a subject suffering from a disease condition characterized by cartilage or joint degradation (e.g. rheumatoid arthritis and/or osteoarthritis) of an effective amount of a compound of the invention for a period of time sufficient to reduce and preferably terminate the self-perpetuating processes responsible for said degradation. A particular embodiment of the method comprises administering of an effective amount of a compound of the invention to a subject patient suffering from or susceptible to the development of osteoarthritis, for a period of time sufficient to reduce or prevent, respectively, cartilage degradation in the joints of said patient, and preferably terminate, the self-perpetuating processes responsible for said degradation. In a particular embodiment said compound may exhibit cartilage anabolic and/or anti-catabolic properties.

Injection dose levels range from about 0.1 mg/kg/h to at least 10 mg/kg/h, all for from about 1 to about 120 h and especially 24 to 96 h. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prophylaxis and/or treatment of long-term conditions, such as degenerative conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of a compound of the invention, with particular doses each providing from about 0.1 to about 10 mg/kg and especially about 1 to about 5 mg/kg.

Transdermal doses are generally selected to provide similar or lower blood levels than are achieved using injection doses.

When used to prevent the onset of a condition, a compound of the invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

A compound of the invention can be administered as the sole active agent or it can be administered in combination with other therapeutic agents, including other compounds that demonstrate the same or a similar therapeutic activity and that are determined to safe and efficacious for such combined administration. In a specific embodiment, co-administration of two (or more) agents allows for significantly lower doses of each to be used, thereby reducing the side effects seen.

In one embodiment, a compound of the invention or a pharmaceutical composition comprising a compound of the invention is administered as a medicament. In a specific embodiment, said pharmaceutical composition additionally comprises a further active ingredient.

In one embodiment, a compound of the invention is co-administered with another therapeutic agent for the treatment and/or prophylaxis of a disease involving inflammation; particular agents include, but are not limited to, immunoregulatory agents e.g. azathioprine, corticosteroids (e.g. prednisolone or dexamethasone), cyclophosphamide, cyclosporin A, tacrolimus, Mycophenolate Mofetil, muromonab-CD3 (OKT3, e.g. Orthocolone®), ATG, aspirin, acetaminophen, ibuprofen, naproxen, and piroxicam.

In one embodiment, a compound of the invention is co-administered with another therapeutic agent for the treatment and/or prophylaxis of arthritis (e.g. rheumatoid arthritis); particular agents include but are not limited to analgesics, non-steroidal anti-inflammatory drugs (NSAIDS), steroids, synthetic DMARDS (for example but without limitation methotrexate, leflunomide, sulfasalazine, auranofin, sodium aurothiomalate, penicillamine, chloroquine, hydroxychloroquine, azathioprine, and ciclosporin), and biological DMARDS (for example but without limitation Infliximab, Etanercept, Adalimumab, Rituximab, and Abatacept).

In one embodiment, a compound of the invention is co-administered with another therapeutic agent for the treatment and/or prophylaxis of proliferative disorders; particular agents include but are not limited to: methotrexate, leukovorin, adriamycin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors (e.g. Lressa®, Tarceva™, Erbitux™), VEGF inhibitors (e.g. Avastin™), proteasome inhibitors (e.g. Velcade™), Glivec® and hsp90 inhibitors (e.g. 17-AAG). Additionally, a compound of the invention may be administered in combination with other therapies including, but not limited to, radiotherapy or surgery. In a specific embodiment the proliferative disorder is selected from cancer, myeloproliferative disease or leukaemia.

In one embodiment, a compound of the invention is co-administered with another therapeutic agent for the treatment and/or prophylaxis of autoimmune diseases, particular agents include but are not limited to: glucocorticoids, cytostatic agents (e.g. purine analogs), alkylating agents, (e.g nitrogen mustards (cyclophosphamide), nitrosoureas, platinum compounds, and others), antimetabolites (e.g. methotrexate, azathioprine and mercaptopurine), cytotoxic antibiotics (e.g. dactinomycin anthracyclines, mitomycin C, bleomycin, and mithramycin), antibodies (e.g. anti-CD20, anti-CD25 or anti-CD3 (OTK3) monoclonal antibodies, Atgam® and Thymoglobuline®), cyclosporin, tacrolimus, rapamycin (sirolimus), interferons (e.g. IFN-β), TNF binding proteins (e.g. infliximab (Remicade™), etanercept (Enbrel™), or adalimumab (Humira™)), mycophenolate, Fingolimod and Myriocin.

In one embodiment, a compound of the invention is co-administered with another therapeutic agent for the treatment and/or prophylaxis of transplantation rejection, particular agents include but are not limited to: calcineurin inhibitors (e.g. cyclosporin or tacrolimus (FK506)), mTOR inhibitors (e.g. sirolimus, everolimus), anti-proliferatives (e.g. azathioprine, mycophenolic acid), corticosteroids (e.g. prednisolone, hydrocortisone), Antibodies (e.g. monoclonal anti-IL-2Rα receptor antibodies, basiliximab, daclizumab), polyclonal anti-T-cell antibodies (e.g. anti-thymocyte globulin (ATG), anti-lymphocyte globulin (ALG)).

In one embodiment, a compound of the invention is co-administered with another therapeutic agent for the treatment and/or prophylaxis of asthma and/or rhinitis and/or COPD, particular agents include but are not limited to: beta2-adrenoceptor agonists (e.g. salbutamol, levalbuterol, terbutaline and bitolterol), epinephrine (inhaled or tablets), anticholinergics (e.g. ipratropium bromide), glucocorticoids (oral or inhaled) Long-acting β2-agonists (e.g. salmeterol, formoterol, bambuterol, and sustained-release oral albuterol), combinations of inhaled steroids and long-acting bronchodilators (e.g. fluticasone/salmeterol, budesonide/formoterol), leukotriene antagonists and synthesis inhibitors (e.g. montelukast, zafirlukast and zileuton), inhibitors of mediator release (e.g. cromoglycate and ketotifen), biological regulators of IgE response (e.g. omalizumab), antihistamines (e.g. ceterizine, cinnarizine, fexofenadine) and vasoconstrictors (e.g. oxymethazoline, xylomethazoline, nafazoline and tramazoline).

Additionally, a compound of the invention may be administered in combination with emergency therapies for asthma and/or COPD, such therapies include oxygen or heliox administration, nebulized salbutamol or terbutaline (optionally combined with an anticholinergic (e.g. ipratropium), systemic steroids (oral or intravenous, e.g. prednisone, prednisolone, methylprednisolone, dexamethasone, or hydrocortisone), intravenous salbutamol, non-specific beta-agonists, injected or inhaled (e.g. epinephrine, isoetharine, isoproterenol, metaproterenol), anticholinergics (IV or nebulized, e.g. glycopyrrolate, atropine, ipratropium), methylxanthines (theophylline, aminophylline, bamiphylline), inhalation anesthetics that have a bronchodilatory effect (e.g. isoflurane, halothane, enflurane), ketamine and intravenous magnesium sulfate.

In one embodiment, a compound of the invention is co-administered with another therapeutic agent for the treatment and/or prophylaxis of inflammatory bowel disease (IBD), particular agents include but are not limited to: glucocorticoids (e.g. prednisone, budesonide) synthetic disease modifying, immunomodulatory agents (e.g. methotrexate, leflunomide, sulfasalazine, mesalazine, azathioprine, 6-mercaptopurine and ciclosporin) and biological disease modifying, immunomodulatory agents (infliximab, adalimumab, rituximab, and abatacept).

In one embodiment, a compound of the invention is co-administered with another therapeutic agent for the treatment and/or prophylaxis of SLE, particular agents include but are not limited to: Disease-modifying antirheumatic drugs (DMARDs) such as antimalarials (e.g. plaquenil, hydroxychloroquine), immunosuppressants (e.g. methotrexate and azathioprine), cyclophosphamide and mycophenolic acid; immunosuppressive drugs and analgesics, such as nonsteroidal anti-inflammatory drugs, opiates (e.g. dextropropoxyphene and co-codamol), opioids (e.g. hydrocodone, oxycodone, MS Contin, or methadone) and the fentanyl duragesic transdermal patch.

In one embodiment, a compound of the invention is co-administered with another therapeutic agent for the treatment and/or prophylaxis of psoriasis, particular agents include but are not limited to: topical treatments such as bath solutions, moisturizers, medicated creams and ointments containing coal tar, dithranol (anthralin), corticosteroids like desoximetasone (Topicort™), fluocinonide, vitamin D3 analogues (for example, calcipotriol), Argan oiland retinoids (etretinate, acitretin, tazarotene), systemic treatments such as methotrexate, cyclosporine, retinoids, tioguanine, hydroxyurea, sulfasalazine, mycophenolate mofetil, azathioprine, tacrolimus, fumaric acid esters or biologics such as Amevive™ Enbrel™, Humira™, Remicade™, Raptiva™ and ustekinumab (a IL-12 and IL-23 blocker). Additionally, a compound of the invention may be administered in combination with other therapies including, but not limited to phototherapy, or photochemotherapy (e.g. psoralen and ultraviolet A phototherapy (PUVA)).

In one embodiment, a compound of the invention is co-administered with another therapeutic agent for the treatment and/or prophylaxis of allergic reaction, particular agents include but are not limited to: antihistamines (e.g. cetirizine, diphenhydramine, fexofenadine, levocetirizine), glucocorticoids (e.g. prednisone, betamethasone, beclomethasone, dexamethasone), epinephrine, theophylline or anti-leukotrienes (e.g. montelukast or zafirlukast), anti-cholinergics and decongestants.

By co-administration is included any means of delivering two or more therapeutic agents to the patient as part of the same treatment regime, as will be apparent to the skilled person. Whilst the two or more agents may be administered simultaneously in a single formulation this is not essential. The agents may be administered in different formulations and at different times.

### General Synthetic Procedures

### General

A compound of the invention can be prepared from readily available starting materials using the following general methods and procedures. It will be appreciated that where typical or preferred process conditions (i.e. reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The following methods are presented with details as to the preparation of a compound of the invention as defined hereinabove and the comparative examples. A compound of the invention may be prepared from known or commercially available starting materials and reagents by one skilled in the art of organic synthesis.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography was performed on silica gel 60 (35-70 μm). Thin layer chromatography was carried out using pre-coated silica gel F-254 plates (thickness 0.25 mm) ¹H NMR spectra were recorded on a Bruker DPX 400 NMR spectrometer (400 MHz). Chemical shifts (δ) for ¹H NMR spectra are reported in parts per million (ppm) relative to tetramethylsilane (δ 0.00) or the appropriate residual solvent peak, i.e. CHCl₃ (δ7.27), as internal reference. Multiplicities are given as singlet (s), doublet (d), triplet (t), quartet (q), multiplet (m) and broad (br). Coupling constants (J) are given in Hz. Electrospray MS spectra were obtained on a Micromass platform LC/MS spectrometer. Columns Used for LCMS analysis: Hichrom, Kromasil Eternity, 2.5 μm C18, 150×4.6 mm, Waters Xbridge 5 μm C18 (2), 250×4.6 mm (ref 86003117), Waters Xterra MS 5 μm C18, 100×4 6 mm (Plus guard cartridge) (ref 186000486), Gemini-NX 3 μm C18 100×3.0 mm (ref 00D-4453-Y0), Phenomenex Luna 5 μm C18 (2), 100×4.6 mm. (Plus guard cartridge) (ref 00D-4252-E0), Kinetix fused core 2.7 μm C18 100×4 6 mm (ref 00D-4462-E0), Supelco, Ascentis® Express C18 (ref 53829-U), or Hichrom Halo C18, 2.7 μm C18, 150×4.6 mm (ref 92814-702). LC-MS were recorded on a Waters Micromass ZQ coupled to a HPLC Waters 2795, equipped with a UV detector Waters 2996. LC were also run on a HPLC Agilent 1100 coupled to a UV detector Agilent G1315A. Preparative HPLC: Waters XBridge Prep C18 5 μm ODB 19 mm ID×100 mm L (Part No. 186002978). All the methods are using MeCN/H₂O gradients. H₂O contains either 0.1% TFA or 0.1% NH₃.

List of abbreviations used in the experimental section:

| | |
|---|---|
| DCM | Dichloromethane |
| DiPEA | N,N-diisopropylethylamine |
| MeCN | Acetonitrile |
| BOC | tert-Butyloxy-carbonyl |
| BINAP | 2,2′-bis(diphenylphosphino)-1,1′-binaphthyl |
| DMA | Dimethylacetamide |
| DMF | N,N-dimethylformamide |
| Cat. | Catalytic amount |
| TFA | Trifluoroacetic acid |
| THF | Tetrahydrofuran |
| NMR | Nuclear Magnetic Resonnance |
| DMSO | Dimethylsulfoxide |
| LC-MS | Liquid Chromatography- Mass Spectrometry |
| ppm | part-per-million |
| Pd/C | Palladium on Charcoal 10% |
| PMB | Para-methoxy-benzyl |
| PyBOP | benzotriazol-1-yl-oxy-tris-pyrrolidino- |
| | phosphonium hexafluoroborate |
| EtOAc | ethyl acetate |
| APCI | atmospheric pressure chemical ionization |
| Rt | retention time |
| s | singlet |
| br s | broad singlet |
| m | multiplet |
| min | minute |
| mL | milliliter |
| μL | microliter |
| g | gram |
| mg | milligram |
| equiv. | equivalents |
| PdCl₂dppf | [1,1′-Bis(diphenylphosphino)ferrocene] |
| | dichloropalladium(II) |
| Pd₂(dba)₃ | Tris(dibenzylideneacetone)dipalladium (0) |
| TEA | Triethylamine |
| MMP | Matrix Metallo Proteinase |
| NHAC | Normal Human Articular Chondrocytes |
| shRNA | short hairpin RNA |
| RNA | Ribonucleic acid |
| Ad-SiRNA | Adenoviral encoded siRNA |
| PBST | Phosphate buffered saline with Tween 3.2 mM |
| | Na₂HPO₄, 0.5 mM KH₂PO₄, 1.3 mM KCl, 135 |
| | mM NaCl, 0.05% Tween 20, pH 7.4 |
| APMA | 4-aminophenylmercuric acetate |
| DMEM | Dulbecco's Modified Eagle Medium |
| FBS | Fetal bovine serum |
| hCAR | human cellular adenovirus receptor |
| 3-MOI | multiplicity of infection of 3 |
| dNTP | deoxyribonucleoside triphosphate |
| QPCR | quantitative polymerase chain reaction |
| cDNA | copy deoxyribonucleic acid |
| GAPDH | Glyceraldehyde phosphate dehydrogenase |
| h | hour |
| mmol | millimoles |
| HPLC | High pressure liquid chromatography |
| NMP | N-Methylpyrrolidone |
| SNAr | Nucleophilic aromatic substitution |

### Synthetic Preparation of the Compounds of the Invention

A compound of the invention can be produced according to the following scheme.

### General Synthetic Method

### Synthesis of Intermediates

### Intermediate 1/Intermediate 2

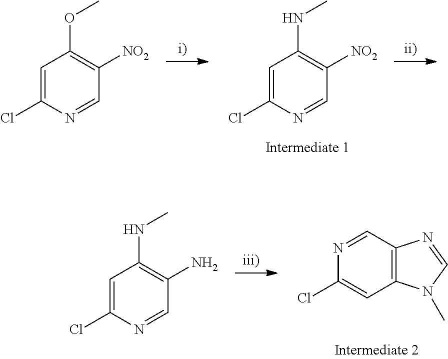

### Step (i): (2-Chloro-5-nitro-pyridin-4-yl)-methyl-amine (Intermediate 1)

To a solution of 2-chloro-4-methoxy-5-nitro-pyridine (0.026 mol) in dry THF (50 mL) at room temperature was added methyl amine (25 mL) (2M in THF). The mixture was allowed to stir for a further 2 h at room temperature. After completion of reaction as seen by TLC and LCMS, solvent was evaporated under reduced pressure to give 5 g of desired Intermediate 1.

¹H-NMR (400 MHz, DMSO-d₆): δ 2.95 (d, 3H), 7.01 (s, 1H), 8.57 (bs, 1H), 8.86, 1H).

Mass (M+1): m/z 188.

### Step (ii): 6-Chloro-N-methyl-pyridine-3,4-diamine

To a stirred solution of intermediate 1 (0.026 mol) in acetic acid (100 mL) was added iron powder (9 g, 0.16 mL) at 50° C. The reaction mixture was then heated at 80° C. for about 1 hr when TLC showed the completion of reaction; it was cooled, filtered and washed with ethyl acetate (3×100 mL). Evaporation of organic layer gave residual mass, which was then neutralized with aq. NaHCO₃ solution and extracted with ethyl acetate (3×100 mL). Combined organic layers were washed with water (2×100 mL) dried over anhydrous sodium sulphate and concentrated under reduced pressure to give the desired compound.

¹H-NMR (400 MHz, DMSO-d₆): δ 2.74 (d, 3H), 4.66 (s, 2H), 6.25 (s, 1H), 7.36 (s, 1H).

Mass (M+1): m/z 158.

### Step (iii) 6-Chloro-1-methyl-1H-imidazo[4,5-c]pyridine: (intermediate 2)

To a stirred solution of 6-Chloro-N-methyl-pyridine-3,4-diamine (22 mmol) in trimethyl orthoformate (25 mL) was added formic acid (1 mL) and was heated at 100° C. for nearly 4 h when TLC showed the completion of reaction. The reaction was allowed to cool to room temperature and water (50 mL) was added and the mixture was extracted with ethyl acetate (4×50 mL), the combined organic layers were washed with aq. NaHCO₃ solution, dried over anhydrous sodium sulphate and concentration under reduced pressure gave the desired product Intermediate 2.

¹H-NMR (400 MHz, DMSO-d₆): δ 3.84 (s, 3H), 7.83 (s, 1H), 8.39 (s, 1H), 8.74 (s, 1H).

Mass (M+1): m/z 168.

### Intermediate 3

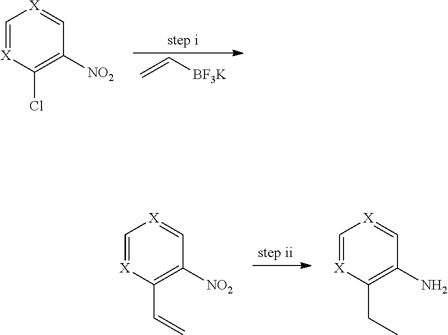

### Step i): Pd-Mediated Coupling

The (hetero)aryl chloride (6.31 mmol), potassium vinyltrifluoroborate (7.57 mmol), cesium carbonate (18.93 mmol) and PdCl₂dppf (0.631 mmol) are suspended in THF (50 mL) and water (5 mL) and the stirred reaction mixture is heated to 75° C. for 1.5 h. The reaction mixture is filtered through Celite and partitioned between diethyl ether and water. The layers are separated and the aqueous layer is extracted with diethyl ether, the organics are combined, dried (MgSO₄) and concentrated in vacuo.

### Step ii): Reduction

To a stirred solution of the nitro compound (6.31 mmol) in MeOH (30 mL) is added ammonium formate (63.1 mmol) and the reaction mixture is degassed (N₂) for 5 min. Pd/C (10% wt, 50% water, 200 mg) is added and the reaction mixture is heated to 75° C. under an atmosphere of N₂ for 4 h. The reaction mixture is filtered through Celite and washed through with DCM and the filtrate washed with water. The aqueous layer is extracted with DCM and the organics are combined, filtered through a hydrophobic filter and concentrated in vacuo. The resulting residue is dissolved in DCM and loaded onto a 20 g SCX column. DCM and MeOH are passed through the column and the target material eluted with 7N NH₃ solution in MeOH:MeOH (1:5). The eluent is concentrated in vacuo.

### Intermediate 5

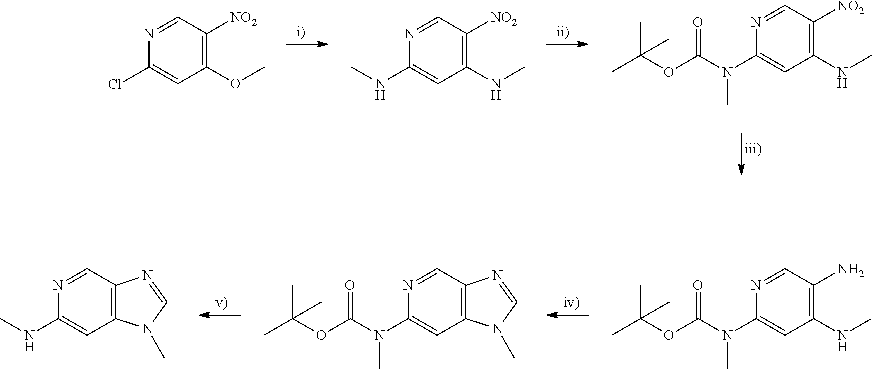

### Step i): N²,N⁴-Dimethyl-5-nitropyridine-2,4-diamine

A mixture of 2-chloro-4-methoxy-5-nitropyridine (9.4 g, 50 mmol) and methylamine (35 wt. % in EtOH) (47 mL, 500 mmol) was stirred at 150° C. (microwave) for 15 min. The reaction mixture was allowed to cool to room temperature. Water (50 mL) was added. The resultant solid material was collected by filtration, washed with MeOH and then Et₂O and dried in vacuo to give the desired compound.

¹H NMR δ (ppm)(CDCl₃): 8.94 (1H, s, ArH), 8.20 (1H, s, NH), 5.33 (1H, s, ArH), 5.24 (1H, s, NH), 3.00 (3H, d, CH₃), 2.97 (3H, d, CH₃).

LCMS (15 cm_Formic_Ascemtis_HPLC_MeCN) Rt 6.24 (min) m/z 183 (MH⁺).

### Step ii): tert-Butyl methyl(4-(methylamino)-5-nitropyridin-2-yl)carbamate

A mixture of N²,N⁴-dimethyl-5-nitropyridine-2,4-diamine (7.3 g, 40 mmol), di-tert-butyldicarbonate (9.6 g, 44 mmol), 4-dimethylaminopyridine (0.49 g, 4 mmol) and MeCN (200 mL) was stirred at room temperature for 16 h. The reaction mixture was concentrated in vacuo. The residue was dissolved in DCM (250 mL) and washed with water (2×150 mL). The organic extract was dried (MgSO₄), filtered and the solvent removed in vacuo. The residue was washed with isohexanes and dried in vacuo to give the desired compound.

¹HNMR δ (ppm)(CDCl₃): 9.05 (1H, s, NH), 8.06 (1H, s, ArH), 7.40 (1H, s, ArH), 3.44 (3H, s, CH₃), 3.05 (3H, d, CH₃), 1.56 (9H, s, CH₃).

LCMS (10 cm_ESI_Formic_MeCN) Rt 3.83 (min) m/z 283 (MH⁺).

### Step iii): tert-Butyl (5-amino-4-(methylamino)pyridin-2-yl)(methyl)carbamate

A mixture of tert-butyl methyl(4-(methylamino)-5-nitropyridin-2-yl)carbamate (9.4 g, 52 mmol) 10 wt. % palladium on carbon (0.94 g, 10 wt. %) and EtOH (208 mL) was stirred at room temperature under a H₂ atmosphere (1 atm.) for 48 h. The reaction mixture was filtered through Celite. The Celite was washed with a mixture of MeOH/DCM (1:1; v/v) (400 mL). The combined filtrate was concentrated in vacuo to give the desired compound.

¹H NMR δ (ppm)(CDCl₃): 7.69 (1H, s, ArH), 6.69 (1H, s, ArH), 4.21 (1H, s, NH), 3.30 (3H, s, CH₃), 2.95 (2H, s, NH₂), 2.90 (3H, d, CH₃), 1.49 (9H, s, CH₃).

LCMS (10 cm_ESI_Formic_MeCN) Rt 2.29 (min) m/z 253 (MH⁺).

### Step iv): tert-Butyl methyl(1-methyl-1H-imidazo[4,5-c]pyridin-6-yl)carbamate

A mixture of tert-butyl (5-amino-4-(methylamino)pyridin-2-yl)(methyl)carbamate (12.6 g, 50 mmol), triethylorthoformate (125 mL, 750 mmol) and formic acid (2.8 mL, 75 mmol) was stirred at 100° C. for 1 h. The reaction mixture was allowed to cool to room temperature and the solvent removed in vacuo. The crude product was purified by column chromatography on silica gel (0-5% MeOH DCM) to give the desired compound.

¹H NMR δ (ppm)(CDCl₃): 8.87 (1H, s, ArH), 7.78 (1H, s, ArH), 7.71 (1H, s, ArH), 3.83 (3H, s, CH₃), 3.46 (3H, s, CH₃), 1.53 (9H, s, CH₃).

LCMS (10 cm_ESI_Formic_MeCN) Rt 2.64 (min) m/z 263 (MH⁺).

### Step v): N,1-Dimethyl-1H-imidazo[4,5-c]pyridin-6-amine

A mixture of tert-butyl methyl(1-methyl-1H-imidazo[4,5-c]pyridin-6-yl)carbamate (5.1 g, 19.4 mmol), a 4 M solution of HCl in dioxane (97 mL, 388 mmol) and MeOH (194 mL) was stirred at room temperature for 72 h. The reaction mixture was concentrated in vacuo. The crude product was purified using a SCX column [eluting with i) DCM, ii) 50% MeOH DCM, iii) 50% (7M NH₃ solution in MeOH)-DCM] to give the desired compound.

¹H NMR δ (ppm)(DMSO-d₆): 8.38 (1H, s, ArH), 7.93 (1H, s, ArH), 6.36 (1H, s, ArH), 6.10 (1H, s, NH), 3.69 (3H, s, CH₃), 2.79 (3H, s, CH₃).

LCMS (15 cm_Bicarb_GeminiNX_HPLC_MeCN) Rt 7.28 (min) m/z 163 (MH⁺).

### Intermediate 6: N-(2-Ethyl-4-(1,2,3,6-tetrahydropyridin-4-yl)phenyl)-N,1-dimethyl-1H-imidazo[4,5-c]pyridin-6-amine

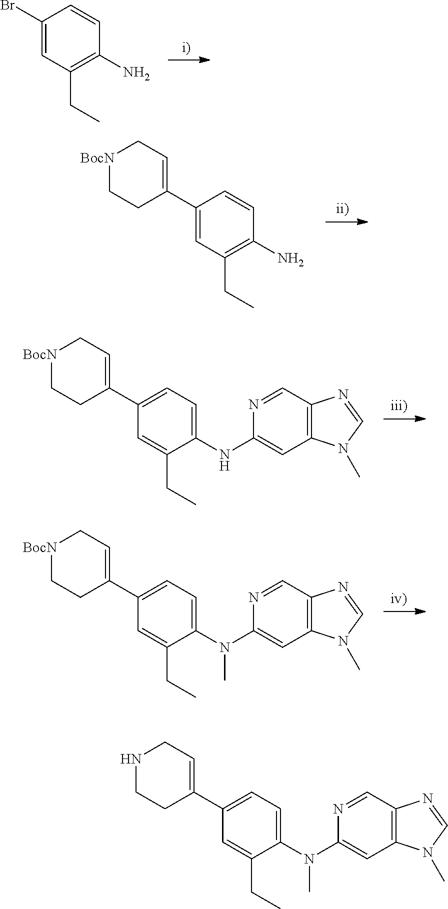

### Step i): tert-Butyl 4-(4-amino-3-ethylphenyl)-5,6-dihydropyridine-1(2H)-carboxylate

4-Bromo-2-ethylaniline (4.96 mL, 35.0 mmol), tert-butyl 4-(4,4,5,5-tetramethyl-1,3,2-dioxaborolan-2-yl)-5,6-dihydropyridine-1(2H)-carboxylate (13 g, 42.0 mmol), [1,1′-bis(diphenylphosphino)ferrocene]dichloropalladium(II) (1.43 g, 1.75 mmol) and cesium carbonate (34.2 g, 105 mmol) were heated at reflux in 1,4-dioxane (180 mL) and water (20 mL) for 18 h. The reaction mixture was cooled to room temperature and filtered through Celite, washed through with DCM. and the organics were washed with water, dried (MgSO₄), filtered and concentrated in vacuo. The resulting residue was purified using column chromatography on silica gel and eluting with 10-20% EtOAc in isohexanes to give the desired compound.

¹H NMR δ (ppm)(DMSO-d₆): 7.02-6.96 (2H, m, ArH), 6.56 (1H, d, ArH), 5.88 (1H, s, CH), 4.89 (2H, s, NH2), 3.94 (2H, s, CH), 3.53-3.47 (2H, m, CH), 2.44 (2H, q, CH2), 2.38 (2H, s, CH), 1.67-1.18 (9H, m, CH3), 1.13 (3H, t, CH3).

### Step ii): tert-Butyl 4-(3-ethyl-4-((1-methyl-1H-imidazo[4,5-c]pyridin-6-yl)amino)phenyl)-5,6-dihydropyridine-1(2H)-carboxylate

To stirred degassed (N₂) 1,4-dioxane (230 mL) was added tert-butyl 4-(4-amino-3-ethylphenyl)-5,6-dihydropyridine-1(2H)-carboxylate (8.25 g, 27.3 mmol), 6-chloro-1-methyl-1H-imidazo[4,5-c]pyridine (4.15 g, 24.8 mmol), tris(dibenzylideneacetone)dipalladium(0) (1.25 g, 1.37 mmol), 2-dicyclohexylphosphino-2′,4′,6′-triisopropylbiphenyl (1.30 g, 2.73 mmol) and sodium tert-butoxide (3.94 g, 30 mmol). The reaction mixture was heated to 100° C. for 1.5 h, cooled to room temperature, filtered through Celite and washed through with DCM. The filtrate was washed with water, dried (MgSO₄), filtered and concentrated in vacuo and the resulting residue was purified by column chromatography using silica gel and eluting with 0-3% MeOH in DCM. The fractions containing product were combined and concentrated in vacuo to give the desired compound.

¹H NMR δ (ppm)(DMSO-d₆): 8.48 (1H, d, NH), 8.04 (1H, s, ArH), 7.89 (1H, s, ArH), 7.56-7.49 (1H, m, ArH), 7.30 (1H, d, ArH), 7.23 (1H, dd, ArH), 6.74-6.71 (1H, m, ArH), 6.10 (1H, s, CH), 4.00 (2H, s, CH), 3.73-3.63 (3H, m, CH3), 3.58-3.52 (2H, m, CH), 2.70-2.59 (2H, m, CH), 2.53-2.46 (2H, s, CH), 1.55-1.35 (9H, m, CH3), 1.18-1.09 (3H, m, CH3).

### Step iii): tert-Butyl 4-(3-ethyl-4-(methyl(1-methyl-1H-imidazo[4,5-c]pyridin-6-yl)amino)phenyl)-5,6-dihydropyridine-1(2H)-carboxylate

Sodium hexamethyldisilazide (1M in THF, 13.5 mL, 13.5 mmol) was added dropwise to a solution of tert-butyl 4-(3-ethyl-4-((1-methyl-1H-imidazo[4,5-c]pyridin-6-yl)amino)phenyl)-5,6-dihydropyridine-1(2H)-carboxylate (5.32 g, 12.3 mmol) in DMF (55 mL) at 0° C. The resulting dark brown solution was stirred at 0° C. for 20 min where iodomethane (0.84 mL, 13.5 mmol) was added dropwise and the reaction mixture was allowed to warm to room temperature over 30 min. The reaction mixture was concentrated in vacuo, dissolved in DCM, washed with water, dried (MgSO₄), filtered and concentrated in vacuo to give the desired compound, which was used in the next step without further purification.

¹H NMR δ (ppm)(CHCl₃-d): 8.72 (1H, s, ArH), 7.61 (1H, s, ArH), 7.43-7.38 (1H, m, ArH), 7.35-7.30 (1H, m, ArH), 7.19-7.15 (1H, m, ArH), 6.13 (1H, s, CH), 5.90 (1H, d, ArH), 4.11 (2H, s, CH), 3.70-3.64 (2H, m, CH), 3.56 (3H, s, CH3), 3.45 (3H, s, CH3), 2.62-2.47 (4H, m, CH), 1.54-1.50 (9H, m, CH3), 1.29-1.11 (3H, m, CH3).

LCMS (10 cm_Formic_ACE 3 C18 AR_HPLC_MeCN) Rt 8.23 (min) m/z 448 (MH⁺).

### Step iv): N-(2-Ethyl-4-(1,2,3,6-tetrahydropyridin-4-yl)phenyl)-N,1-dimethyl-1H-imidazo[4,5-c]pyridin-6-amine

tert-Butyl 443 ethyl-4-(methyl(1-methyl-1H-imidazo[4,5-c]pyridin-6-yl)amino)phenyl)-5,6-dihydropyridine-1(2H)-carboxylate (2.5 g, 5.59 mmol) was stirred in DCM (25 mL) and trifluoroacetic acid (5 mL) at room temperature for 1 d. The reaction mixture was concentrated in vacuo, dissolved in DCM and loaded onto a 10 g SCX column, which was washed with MeOH and eluted with 7N NH₃ in MeOH:MeOH (1:5). The eluent was concentrated in vacuo to give the desired compound.

¹H NMR δ (ppm)(DMSO-d₆): 8.52-8.45 (1H, m, ArH), 7.98 (1H, s, ArH), 7.42 (1H, d, ArH), 7.34 (1H, dd, ArH), 7.13 (1H, d, ArH), 6.25 (1H, s, ArH), 6.13 (1H, s, CH), 4.12 (1H, s, NH), 3.59 (3H, s, CH3), 3.41-3.38 (2H, m, CH), 3.17 (3H, s, CH3), 2.97-2.88 (2H, m, CH), 2.45 (2H, q, CH2), 2.39 (2H, s, CH), 1.17-1.04 (3H, m, CH3).

### Intermediate 7: 3-Ethyl-4-(methyl(1-methyl-1H-imidazo[4,5-c]pyridin-6-yl)amino)phenyl trifluoromethanesulfonate

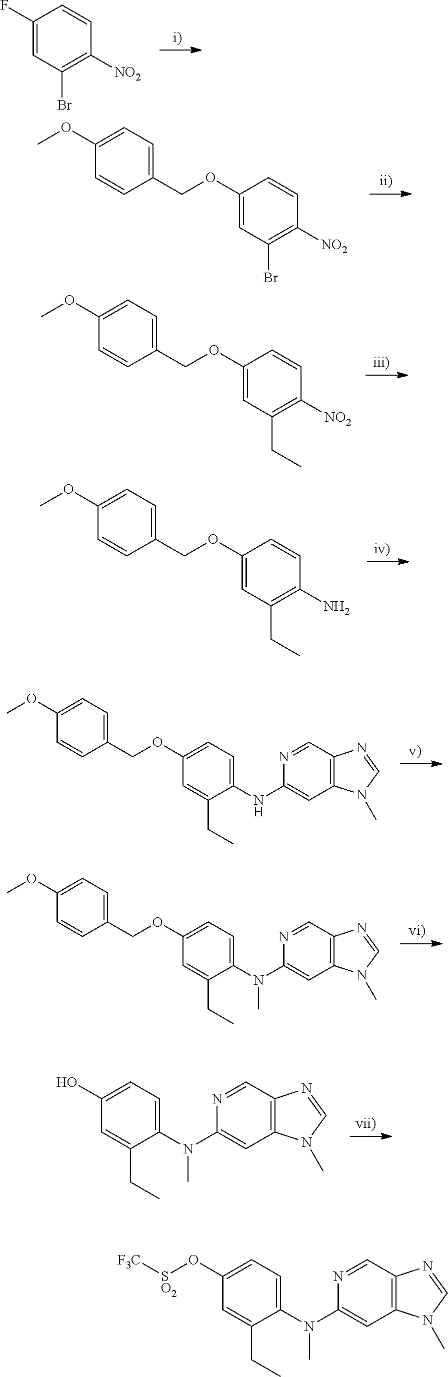

### Step i): 2-Bromo-4-((4-methoxybenzyl)oxy)-1-nitrobenzene

1-Bromo-5-fluoro-2-nitrobenzene (3 g, 13.6 mmol), 4-methoxybenzyl alcohol (3.4 mL, 27.3 mmol) and tetrabutylammonium hydrogen sulfate (462 mg, 1.36 mmol) were stirred vigorously in NaOH (1 M aqueous solution, 60 mL) and DCM (60 mL) for 1 d. The layers were separated and the aqueous was extracted with DCM. The combined organics were dried (hydrophobic frit) and concentrated in vacuo. The residue was purified by column chromatography using silica gel and eluting with DCM. The fractions containing product were combined and concentrated in vacuo to give the desired compound.

¹H NMR δ (ppm)(DMSO-d₆): 8.08 (1H, dd, ArH), 7.53 (1H, d, ArH), 7.44-7.36 (2H, m, ArH), 7.28-7.17 (1H, m, ArH), 6.99-6.94 (2H, m, ArH), 5.18 (2H, s, CH2), 3.77 (3H, s, CH3).

### Step ii): 2-Ethyl-4-((4-methoxybenzyl)oxy)-1-nitrobenzene

2-Bromo-4-((4-methoxybenzyl)oxy)-1-nitrobenzene (2.2 g, 6.5 mmol), ethyl boronic acid (594 mg, 7.8 mmol), [1,1′-bis(diphenylphosphino)ferrocene]dichloropalladium(II) (265 mg, 0.33 mmol) and K₃PO₄ (2.76 g, 13 mmol) were heated in 1,4-dioxane (30 mL) at reflux for 1 d. The reaction mixture was cooled to room temperature and filtered through Celite, washed through with DCM and the organics were washed with water, dried (hydrophobic frit) and concentrated in vacuo to give the desired compound, which was used in the next step without further purification.

### Step 2-Ethyl-4-((4-methoxybenzyl)oxy)aniline

2-Ethyl-4-((4-methoxybenzyl)oxy)-1-nitrobenzene (2.7 mmol), NH₄Cl (217 mg, 4.05 mmol) and iron powder (753 mg, 13.5 mmol) were suspended in THF (3 mL), ethanol (3 mL) and water (1 mL). The stirred reaction mixture was heated to 90° C. for 1 h. The reaction mixture was filtered through Celite, washed through with DCM and concentrated in vacuo. The resulting residue was partitioned between DCM and sat. aq. NaHCO₃ solution. The layers were separated and the aqueous layer extracted with DCM, the organics were combined and dried (hydrophobic filter) and concentrated in vacuo. The resulting residue was purified using column chromatography on silica gel.

¹H NMR δ (ppm)(DMSO-d₆): 7.38-7.30 (2H, m, ArH), 6.94-6.89 (2H, m, ArH), 6.65-6.51 (3H, m, ArH), 4.86 (2H, s, CH2), 4.38 (2H, s, NH2), 3.75 (3H, s, CH3), 2.41 (2H, q, CH2), 1.11 (3H, t, CH2).

### Step iv): N-(2-Ethyl-4-((4-methoxybenzyl)oxy)phenyl)-1-methyl-1H-imidazo[4,5-c]pyridin-6-amine

Prepared using method A to give the desired compound.

¹H NMR δ (ppm)(DMSO-d₆): 8.42 (1H, d, ArH), 7.99-7.94 (1H, m, ArH), 7.71 (1H, s, NH), 7.44-7.36 (2H, m, ArH), 7.28-7.20 (1H, m, ArH), 7.00-6.90 (3H, m, ArH), 6.84 (1H, dd, ArH), 6.33 (1H, d, ArH), 5.01 (2H, s, CH2), 3.77 (3H, s, CH3), 3.63 (3H, s, CH3), 2.56 (2H, m, CH2), 1.09 (3H, t, CH3).

### Step v): N-(2-Ethyl-4-((4-methoxybenzyl)oxy)phenyl)-N,1-dimethyl-1H-imidazo[4,5-c]pyridin-6-amine

Prepared using method B to give the desired compound. ¹H NMR δ (ppm)(DMSO-d₆): 8.53-8.46 (1H, m, ArH), 7.42 (2H, d, ArH), 7.11-6.86 (6H, m, ArH), 6.03 (1H, s, ArH), 5.04 (2H, s, CH2), 3.88-3.63 (3H, m, CH3), 3.57 (3H, s, CH3), 3.33 (3H, s, CH3), 2.41 (2H, q, CH2), 1.14-1.04 (3H, m, CH3).

### Step vi): 3-Ethyl-4-(methyl(1-methyl-1H-imidazo[4,5-c]pyridin-6-yl)amino)phenol

N-(2-Ethyl-4-(4-methoxybenzyl)oxy)phenyl)-N,1-dimethyl-1H-imidazo[4,5-c]pyridin-6-amine (436 mg, 1.08 mmol) was stirred in DCM (3 mL) and trifluoroacetic acid (3 mL) at room temperature for 2 h. The reaction mixture was concentrated in vacuo and loaded onto a SCX column in DCM. The column was washed with MeOH and eluted with 7 N NH₃ solution in MeOH:MeOH (1:5). The eluent was concentrated in vacuo to give the desired compound.

¹H NMR δ (ppm)(DMSO-d₆): 9.41 (1H, s, OH), 8.47 (1H, s, ArH), 7.92 (1H, s, ArH), 6.95 (1H, d, ArH), 6.75 (1H, d, ArH), 6.67 (1H, dd, ArH), 5.94 (1H, s, ArH), 3.53 (3H, s, CH3), 3.31 (3H, s, CH3), 2.34 (2H, q, CH2), 1.04 (3H, t, CH3).

LCMS (10 cm_ESCI_Formic_MeCN) Rt 2.24 (min) m/z 283 (MH⁺).

### Step vii): 3-Ethyl-4-(methyl(1-methyl-1H-imidazo[4,5-c]pyridin-6-yl)amino)phenyltrifluoromethanesulfonate

3-Ethyl-4-(methyl(1-methyl-1H-imidazo[4,5-c]pyridin-6-yl)amino)phenol (230 mg, 0.82 mmol), N-phenylbis(trifluoromethanesulfonamide) (321 mg, 0.90 mmol) and triethylamine (0.14 mL, 0.98 mmol) were stirred in DMF (5 mL) at room temperature for 1 d. Further N-phenylbis(trifluoromethanesulfonamide) (88 mg, 0.25 mmol) and triethylamine (34 μL, 0.25 mmol) were added and the reaction mixture stirred at room temperature for 2 h. Water and DCM were added and the aqueous was extracted with DCM. The combined organics were washed with water, dried (hydrophobic frit) and concentrated in vacuo to give the desired compound, which was used in the next step without further purification.

### Step viii): (4-Aminomethyl-2-ethyl-phenyl)-(1-methyl-1H-imidazo[4,5-c]pyridin-6-yl)-amine (Intermediate 7)

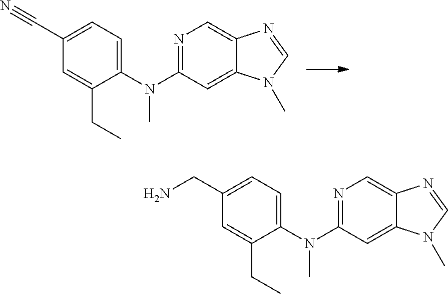 [SMILES: CCc1cc(C#N)ccc1N(C)c1cc2c(cn1)ncn2C ; CCc1cc(CN)ccc1N(C)c1cc2c(cn1)ncn2C]

To a solution of compound 82 (5 g) in dry MeOH (500 mL) was added NiCl₂.6H₂O (1 equiv, 4.016 g) and TFA (8 equiv, 10.52 mL). The mixture was cooled to 0° C. and NaBH₄ (8 equiv, 5.19 g) was added. The reaction mixture was allowed to stir for 2 h. The reaction was monitored by LCMS. The reaction mixtures were filtered over Celite, the filtrate was dried under reduced pressure. EtOAc and aqueous solution of NaOH (2M) were added followed by the separation of the two phases. The formed solid was filtered and washed with MeOH. The combined organic phases were dried. The crude material was purified by flash chromatography to give the desired product.

### General Synthetic Methods for Preparation of the Compounds of the Invention

### Method A: Buchwald Reaction

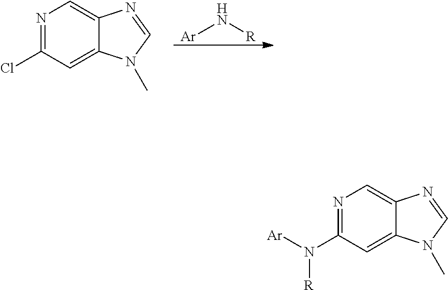

The corresponding amine (0.58 mmol) is added to a solution of intermediate 2 (0.45 mmol) and cesium carbonate (0.62 mmol) in Dioxane (3 mL). Degassing is done for 5 min, followed by addition of a solution previously sonicated for 10 min of Xantphos (0.06 equiv), Pd₂(dba)₃ (0.03 equiv) in dioxane (1 mL) under nitrogen. The reaction is heated at 110° C. for 16 h. After completion, water and DCM are added and this mixture is filtered through a phase separator. The organic layers are concentrated under reduced pressure. The crude product is purified by preparative HPLC to afford desired compound.

### Method A′

To a solution of intermediate 2 (14.9 mmol) in dioxane (100 mL) is added the corresponding aniline (22.4 mmol), cesium carbonate (22.4 mmol), BINAP (0.9 mmol) and tris(dibenzylideneacetone)dipalladium (0.45 mmol). The reaction mixture is refluxed for 15 h, filtered on Celite, evaporated to dryness and purified on silica gel to give the desired compound.

### Method A″

A mixture of intermediate 2 (84 mg, 0.5 mmol), 7-methyl-2,3-dihydrobenzo[b][1,4]dioxin-6-amine (1.5 equiv.), dibenzylideneacetone bis(triphenylphiosphine) palladium (0) (5 mol. %), 4,5-bis(diphenylphosphino)-9,9-dimethylxanthene (10 mol. %), sodium tert-butoxide (1.5 equiv.) and DMF (0.33 M) is stirred at 120° C. for 16 h. The reaction mixture is allowed to cool to room temperature, filtered and the solvent removed in vacuo. The crude product is purified by preparative HPLC to give the desired compound.

### Method B: Alkylation

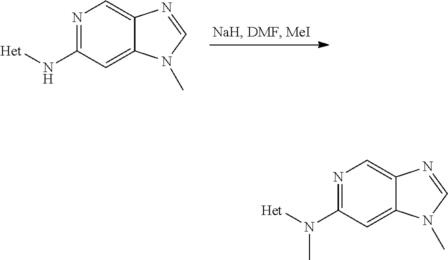

To a suspension of sodium hydride (60% in oil, 1.25 equiv) in DMF (0.5 mL) is added dropwise the imidazopyrine (0.238 mmol, 1 equiv) previously dissolved in DMF (0.5 mL). The reaction is stirred at room temperature for 20 min, then methyl iodide (1.1 equiv) is added dropwise. The reaction mixture is stirred at room temperature for 2 h, then the reaction mixture is diluted with EtOAc and washed with water. The organic layer is dried over sodium sulphate, filtered, and concentrated in vacuo. The crude is then purified by flash column chromatography on silica gel using DCM/MeOH (gradient 100 to 95/5) to give the desired compound.

### Method B′: Alkylation

### General Procedure for N-Methylation

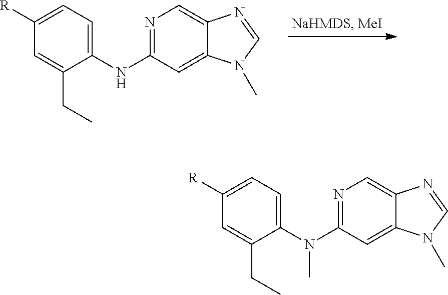

Sodium hexamethyldisilazide (1.1 equiv.) was added dropwise to a solution of the aniline (1 equiv.) in dry DMF at 0° C. After 10-30 min at 0° C., iodomethane (1.1 equiv.) was added and the reaction mixture was allowed to warm to room temperature over 30 min 2 h. The reaction mixture was concentrated in vacuo and purified by preparative HPLC or column chromatography using silica gel to give the desired compound.

### Method C: Reductive Amination

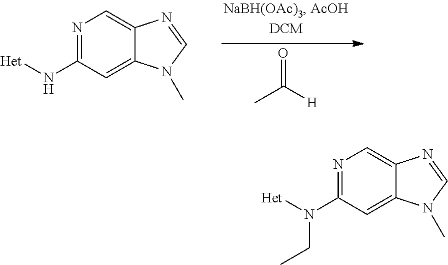

To a solution of imidazopyrine (0.160 mmol, 1 equiv) in DCM (6.5 mL) are added acetic acid

(2 drops), the acetaldehyde (4.0 equiv) and the sodium triacetoxyborohydride (4.0 equiv). The reaction is stirred overnight at room temperature. Then the reaction mixture is diluted with DCM and washed with NaHCO₃ and then with brine. The organic layer is dried over sodium sulphate, filtered, and concentrated in vacuo. The crude is then purified by flash column chromatography on silica gel using DCM/MeOH (gradient 100 to 95/5) to give the desired compound.

### Method D: 6-oxy-1-methyl-1H-imidazo[4,5-c]pyridines

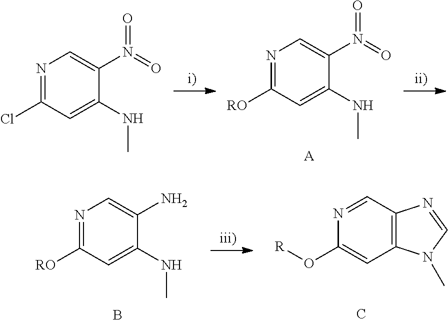

### Step i)

A mixture of sodium hydride (60% dispersion in mineral oil, 1.1 equiv.), ROH (1.1 equiv.) and DMF (0.25 M with respect to amine) is stirred at room temperature for 5 min. A solution of 2-chloro-N-methyl-5-nitropyridin-4-amine (1 equiv.) in DMF (0.25 M) is added and the reaction mixture stirred at room temperature for 4 h. The reaction mixture is concentrated in vacuo. The residue is dissolved in DCM and washed with 10% w/v citric acid (aq.) and water. The aqueous phases are combined and extracted with DCM. The combined organic extracts are dried (phase separating cartridge) and the solvent removed in vacuo. The crude product is purified by column chromatography using silica gel and eluting with 0-100% EtOAc in isohexanes to give the desired compound A.

### Step ii)

A solution of compound A (1 equiv.) in a MeOH/DCM (4:3; v/v) (0.02 M) is passed through an H-Cube (45° C., 1 Bar, flow rate: 1 mLmin⁻¹) fitted with a 10 wt. % palladium on carbon. The resultant solution is concentrated in vacuo to give the desired compound B.

### Step iii)

A mixture of compound B (1 equiv.), triethyl orthoformate (20 equiv.) and formic acid (1.5 equiv.) is stirred at 105° C. for 20 min. The reaction mixture is allowed to cool to room temperature and concentrated in vacuo. The crude product is purified by preparative HPLC to give the desired compound C.

### Method E: General procedure for N-Alkylation

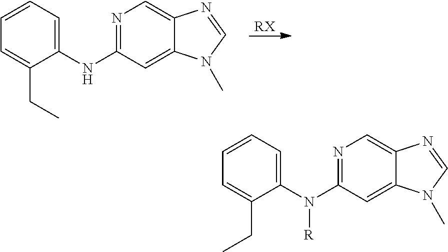

A solution of N-(2-ethylphenyl)-1-methyl-1H-imidazo[4,5-c]pyridin-6-amine (0.79 mmol) in DMF (3 mL) is added dropwise to a suspension of sodium hydride (60% dispersion in mineral oil, 1.19 mmol) in DMF (1 mL) and stirred at room temperature for 1 h. The haloalkane R—X (0.95 mmol) is added and the reaction mixture stirred for 3 h at room temperature. The reaction mixture is partitioned between DCM and water, the layers are separated and the aqueous layer extracted with DCM. The organics are combined, filtered through a hydrophobic filter then concentrated in vacuo. The resulting residue is purified by preparative HPLC.

### Method F: Formation of phenoxy derivatives

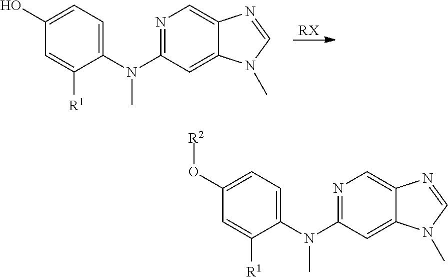

To a solution of compound 28 described below (1 equiv) in aceton (4 mL) is added carbonate potassium (2 equiv) and the desired haloalkane R—X (1.5 equiv), wherein R is alkyl, and X is Cl, Br or I. The mixture is heated at 40° C. for 24 h and purified by preparative HPLC to give the desired compound.

### Method G: General Procedure for the Synthesis of 4-Position Heterocycles

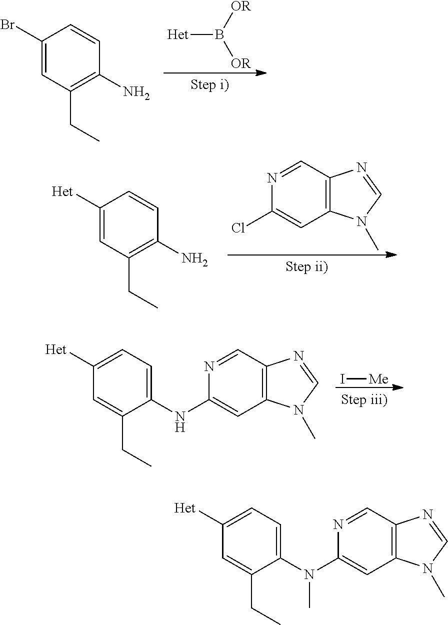

### Step i 4-heterocyclyl-2-ethylaniline

4-Bromo-2-ethylaniline (0.5 g, 2.5 mmol), boronic acid or boronate pinacol ester (1.2 equiv., 3 mmol) and cesium carbonate (2.4 g, 7.5 mmol) are suspended in 1,4-dioxane (10 mL) and water (2 mL) and the reaction mixture is then degassed (N₂) for 5 min. [1,1′-bis(diphenylphosphino)ferrocene]dichloropalladium(II) (0.102 g, 0.125 mmol) is then added and the reaction mixture is heated to 95° C. for 1 day. The reaction is cooled to room temperature and filtered through Celite, washed through with DCM and the organics are washed with water, the layers are separated and the aqueous layer further extracted with DCM. The organics are combined, dried (hydrophobic filter) and concentrated in vacuo. The resulting residue is dissolved in DCM and allowed to load under gravity onto a 10 g SCX column, washed with DCM and MeOH and eluted with 7 N NH₃ solution in MeOH:MeOH (1:5). The eluent is concentrated in vacuo and the resulting residue is purified using column chromatography on silica gel. The fractions containing product are combined and concentrated in vacuo to give the desired compound.

### Step ii): (2-Ethyl-4-heterocyclyl-phenyl)-(1-methyl-1H-imidazo[4,5-c]pyridin-6-yl)-amine

To stirred degassed (N₂) 1,4-dioxane (10 mL) is added the product of step i) above (2.00 mmol), 6-chloro-1-methyl-1H-imidazo[4,5-c]pyridine (303 mg, 1.8 mmol), tris(dibenzylideneacetone)dipalladium(0) (82 mg, 0.09 mmol), 2-dicyclohexylphosphino-2′,4′,6′-triisopropylbiphenyl (86 mg, 0.18 mmol) and sodium tert-butoxide (260 mg, 2.7 mmol). The reaction mixture is heated to 100° C. for 1 d, cooled to room temperature, filtered through Celite and washed through with DCM. The reaction mixture is washed with water and the layers are separated and the aqueous layer further extracted with DCM. The organics are combined, dried (hydrophobic filter) and concentrated in vacuo and the resulting residue is purified by column chromatography using silica gel. The fractions containing product are combined and concentrated in vacuo to give the desired compound.

### Step iii): Methylation Reaction

Sodium hydride (60% dispersion in mineral oil, 36 mg, 0.9 mmol) is added to a stirred solution of the product of step ii) above (0.60 mmol) in DMF (3 mL) at 0° C. and allowed to warm to room temperature over 1 h. Iodomethane (42 μL, 0.66 mmol) is added and the reaction mixture is stirred at room temperature for 1 h. The reaction mixture is partitioned between DCM and water, the layers are separated and the aqueous layer further extracted with DCM. The organics are combined and dried (hydrophobic filter), concentrated in vacuo and the resulting residue is purified by preparative HPLC to give the desired compound.

### Method H: General Procedure for Amide Formation

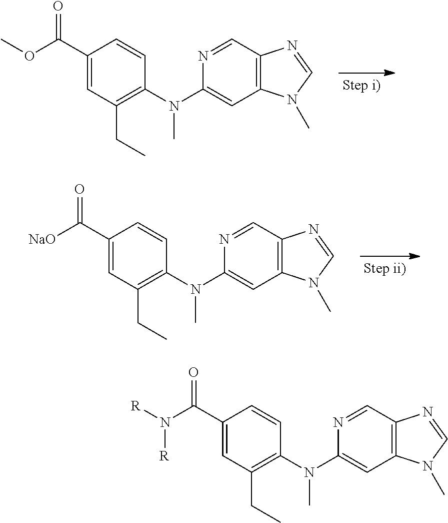

### Step i) Sodium 3-ethyl-4-(methyl(1-methyl-1H-imidazo[4,5-c]pyridin-6-yl)amino)benzoate

In a microwave tube are added methyl 3-ethyl-4-(methyl(1-methyl-1H-imidazo[4,5-c]pyridin-6-yl)amino)benzoate (116 mg, 0.358 mmol) and sodium hydroxide (15 mg, 0.375 mmol) in THF (3 mL) and water (0.5 mL). The reaction vessel is sealed and heated to 100° C. for 20 min under microwave irradiation. The reaction mixture is concentrated in vacuo to give the desired compound, which is used in the next step without further purification.

### Step ii) General Procedure for Amide Formation

To a stirred solution of sodium 3-ethyl-4-(methyl(1-methyl-1H-imidazo[4,5-c]pyridin-6-yl)amino)benzoate (30 mg, 0.086 mmol) and (2-(7-aza-1H-benzotriazole-1-yl)-1,1,3,3-tetramethyluronium hexafluorophosphate) (36 mg, 0.095 mmol) in DMF (1 mL) is added Et₃N (36 mL, 0.26 mmol) followed by the amine (2 equiv., 0.172 mmol). The reaction mixture is stirred at room temperature for 1 d and then purified by preparative HPLC, to obtain the desired compound.

### Method I

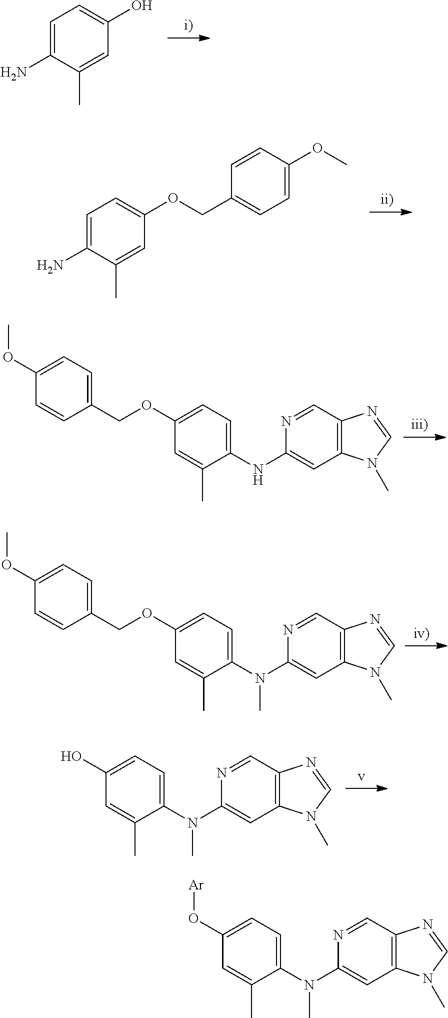

### Step i: 4-(4-Methoxybenzyloxy)-2-methylaniline

1-(Chloromethyl)-4-methoxybenzene (17 g, 0.1 mol) is added to a stirred suspension of cesium carbonate (48.9 g, 0.15 mol) and 4-amino-3-methylphenol (12.2 g, 0.1 mol) in DMF (200 mL) and the resulting suspension is stirred at room temperature for 1 d. The mixture is concentrated in vacuo and the residue is treated with water and extracted with DCM. The combined extracts are dried (MgSO₄), filtered, concentrated in vacuo and purified by column chromatography using silica gel (40% to 75% EtOAc in isohexanes) to give the desired compound.

¹H NMR δ (ppm)(DMSO-d₆): 7.32 (2H, d, ArH), 6.97-6.89 (2H, m, ArH), 6.64 (1H, d, ArH), 6.61-6.50 (2H, m, ArH), 4.85 (2H, s, CH₂), 4.36 (2H, s, NH₂), 3.75 (3H, s, CH₃), 2.03 (3H, s, CH₃).

### Step ii: N-(4-(4-Methoxybenzyloxy)-2-methylphenyl)-1-methyl-1H-imidazo[4,5-c]pyridin-6-amine

6-Chloro-1-methyl-1H-imidazo[4,5-c]pyridine (2.52 g, 0.015 mol) is stirred with 4,5-bis(diphenylphosphino)-9,9-dimethylxanthene (0.86 g, 0.0015 mol), sodium-tert-butoxide (2.16 g, 0.0225 mol) and 4-(4-methoxybenzyloxy)-2-methylaniline (3.68 g, 0.015 mol) in dry dioxane (50 mL) under N₂ (g) for 30 min. Tris(dibenzylideneacetone)dipalladium(0) (0.68 g, 0.00075 mol) is added and the mixture is stirred at 100° C. for 4.5 h. The mixture is concentrated in vacuo and the residue is treated with water (50 mL) and extracted into EtOAc (2×300 mL). The combined extracts are washed with brine, dried (MgSO₄), filtered and concentrated in vacuo to give a solid which is recrystalised from isopropanol to give the desired compound.

¹H NMR δ (ppm)(DMSO-d₆): 8.43 (1H, d, ArH), 7.98 (1H, s, NH), 7.75 (1H, s, ArH), 7.39 (2H, d, ArH), 7.27 (1H, d, ArH), 7.00-6.90 (3H, m, ArH), 6.82 (1H, dd, ArH), 6.38 (1H, d, ArH), 5.00 (2H, s, CH), 3.77 (3H, s, CH₃), 3.64 (3H, s, CH₃), 2.17 (3H, s, CH₃).

LCMS (15 cm_Bicarb_GeminiNX_HPLC_MeCN) Rt 9.63 (min) m/z 375 (MH⁺).

### Step iii: N-(4-(4-Methoxybenzyloxy)-2-methylphenyl)-N,1-dimethyl-1H-imidazo[4,5-c]pyridin-6-amine

Sodium hydride (0.38 g of 60% dispersion in mineral oil, 9.4 mmol) is added to a stirred solution of N-(4-(4-methoxybenzyloxy)-2-methylphenyl)-1-methyl-1H-imidazo[4,5-c]pyridin-6-amine (3.25 g, 8.6 mmol) in dry DMF (80 mL) under nitrogen. The reaction mixture is stirred at room temperature for 1 h before the addition of iodomethane (1.83 g, 12.9 mmol) and stirring is then continued for 5 h. The reaction mixture is concentrated in vacuo and the residue is treated with water (50 mL) and extracted with EtOAc, the combined extracts are dried (MgSO₄), filtered and concentrated in vacuo to give the desired compound, which is used without further purification.

For analytical purposes, a sample of this material (100 mg) is purified by preparative HPLC.

¹H NMR δ (ppm)(DMSO-d₆): 8.49 (1H, d, ArH), 7.96 (1H, s, ArH), 7.41 (2H, d, ArH), 7.11 (1H, d, ArH), 7.02-6.90 (4H, m, ArH), 6.04 (1H, s, ArH), 5.03 (2H, s, CH2), 3.77 (3H, s, CH₃), 3.57 (3H, s, CH₃), 3.32 (3H, d, CH₃), 2.12-1.99 (3H, m, CH₃).

LCMS (15 cm_Bicarb_GeminiNX_HPLC_MeCN) Rt 10.11 (min) m/z 389 (MH⁺).

### Step iv: 3-Methyl-4-(methyl(1-methyl-1H-imidazo[4,5-c]pyridin-6-yl)amino)phenol

N-(4-(4-Methoxybenzyloxy)-2-methylphenyl)-N,1-dimethyl-1H-imidazo[4,5-c]pyridin-6-amine (3.2 g) is dissolved in DCM (15 mL) and trifluoroacetic acid (15 mL) is added. The resulting solution is allowed to stand for 4 h. The reaction is concentrated in vacuo and the residue is loaded onto a 70 g SCX cartridge and washed with MeOH until the washings reached a neutral pH. The product is eluted with MeOHic ammonia to give the desired compound.

For analytical purposes, a sample of this material (100 mg) is purified by preparative HPLC.

¹H NMR δ (ppm)(DMSO-d₆): 9.38 (1H, s, OH), 8.49 (1H, d, ArH), 7.94 (1H, s, ArH), 6.98 (1H, d, ArH), 6.74 (1H, d, ArH), 6.68 (1H, dd, ArH), 5.97 (1H, d, ArH), 3.55 (3H, s, CH₃), 3.37-3.24 (3H, m, CH₃), 1.99 (3H, s, CH₃).

LCMS (10 cm_Formic_ACE-AR_HPLC_MeOH_Slow) Rt 8.92 (min) m/z 269 (MH⁺).

### Step v: N-(4-Methoxy-2-methylphenyl)-N,1-dimethyl-1H-imidazo[4,5-c]pyridin-6-amine

The desired aryl halide (Ar—X, wherein X is Cl, Br, or I) (0.8 mmol) is added to a stirred suspension of 3-methyl-4-(methyl(1-methyl-1H-imidazo[4,5-c]pyridin-6-yl)amino)phenol (0.4 mmol) and cesium carbonate (0.195 g, 0.6 mmol) in DMF (1 mL). The mixture is stirred at 150° C. for 15 min in a microwave, filtered and purified by preparative HPLC to give the desired compound.

### Method J

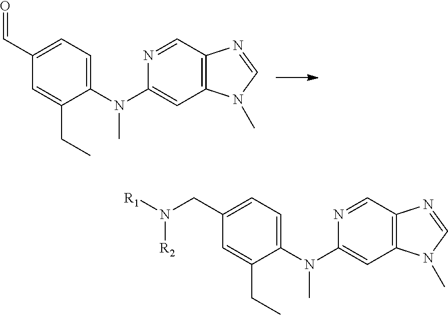

3-Ethyl-4-(methyl(1-methyl-1H-imidazo[4,5-c]pyridin-6-yl)amino)benzaldehyde (100 mg, 0.34 mmol), the amine (1.02 mmol) and (polystyrylmethyl)trimethylammoniumcyanoborohydride (4 mmol/g loading, 250 mg, 1.02 mmol) were shaken in DCM (3 mL) and acetic acid (0.3 mL) for 1 d. The beads were filtered off and the filtrate was concentrated in vacuo and purified by preparative HPLC.

### Method K

### General Procedure for Amide Coupling

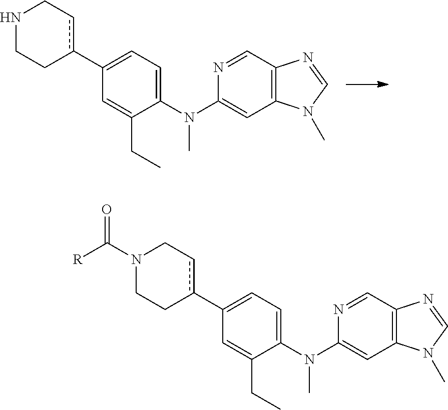

Intermediate 6 (1 equiv.), the acid (1.1 equiv.), (2-(7-aza-1H-benzotriazole-1-yl)-1,1,3,3-tetramethyluronium hexafluorophosphate) (1.2 equiv.) and diisopropylethylamine (4 equiv.) were stirred in DMF at room temperature for 1 to 24 h. The crude reaction mixture was purified by preparative HPLC to give the desired compound.

### Method L

### General Procedure for Oxadiazole

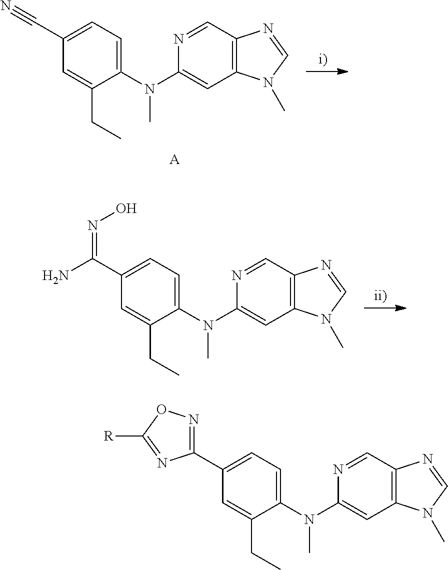

A is synthesized using commercially available 4-amino-3-ethylbenzonitrile and methods A and B.

### Step i: 3-Ethyl-N′-hydroxy-4-(methyl(1-methyl-1H-imidazo[4,5-c]pyridin-6-yl)amino)benzimidamide

3-Ethyl-4-(methyl(1-methyl-1H-imidazo[4,5-c]pyridin-6-yl)amino)benzonitrile (861 mg, 2.96 mmol) and hydroxylamine (50% solution in water, 0.5 mL) are heated at reflux in ethanol (10 mL) for 2 h. The reaction mixture is cooled to room temperature and DCM and water are added. The aqueous layer was extracted with DCM and the combined organics are washed with water, dried (hydrophobic filter) and concentrated in vacuo to give the desired compound, which is used in the next step without further purification.

### Step ii: Oxadiazole Formation

3-Ethyl-N′-hydroxy-4-(methyl(1-methyl-1H-imidazo[4,5-c]pyridin-6-yl)amino)benzimidamide (100 mg, 0.31 mmol), the acid (0.34 mmol), (2-(7-aza-1H-benzotriazole-1-yl)-1,1,3,3-tetramethyluronium hexafluorophosphate) (177 mg, 0.47 mmol) and diisopropylethylamine (0.22 mL, 1.24 mmol) are stirred in DMF (3 mL) at 55° C. for 1 to 3 d. The crude reaction mixture is purified by preparative HPLC to give the desired compound.

### Method M

### General Procedure for Conversion of Nitrile to Primary Amide

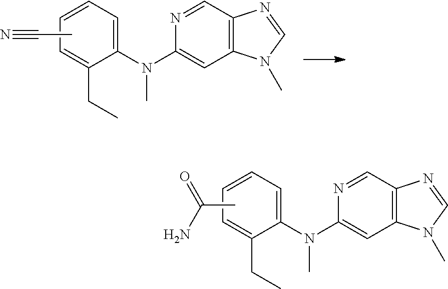

The nitrile (100 mg, 0.34 mmol) is heated in NaOH (1 M, aqueous, 2 mL) and MeOH (2 mL) at 45° C. for 2 d. The reaction mixture is cooled to room temperature and DCM and water are added. The aqueous layer is extracted with DCM and the combined organics are washed with water, dried (hydrophobic filter), concentrated in vacuo and purified by preparative HPLC to give the desired compound.

### Method N

### General Procedure for Suzuki

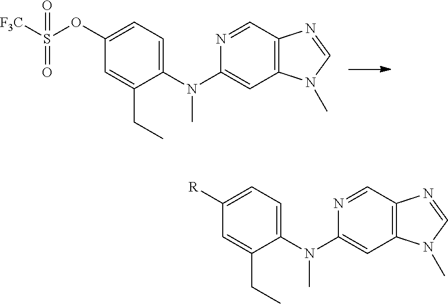

3-Ethyl-4-(methyl(1-methyl-1H-imidazo[4,5-c]pyridin-6-yl)amino)phenyl trifluoromethanesulfonate (129 mg, 0.31 mmol), the boronic acid or boronic acid pinacol ester (0.37 mmol), [1,1′-bis(diphenylphosphino)ferrocene]dichloropalladium(II) (13 mg, 0.016 mmol) and cesium carbonate (303 mg, 0.93 mmol) are heated at reflux in 1,4-dioxane (3 mL) and water (1 mL) for 2 h. The reaction mixture is cooled to room temperature, filtered through Celite and washed through with DCM. The organics are washed with water, dried (hydrophobic frit), concentrated in vacuo and purified by preparative HPLC to give the desired compound.

### Method 0

### General Procedure for Imidazole Formation

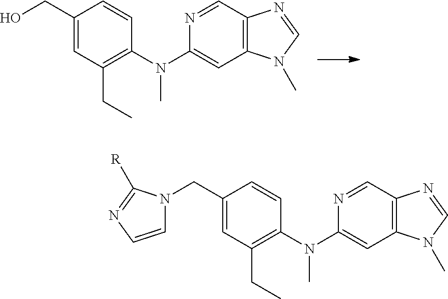

(3-Ethyl-4-(methyl(1-methyl-1H-imidazo[4,5-c]pyridin-6-yl)amino)phenyl)MeOH (40 mg, 0.14 mmol) is dissolved in DMF (2 mL). The bis-(imidazo)methanone (0.68 mmol) is added and the reaction mixture is heated to 100° C. for 5 h. The reaction is allowed to cool to room temperature and the crude material is purified by preparative HPLC to give the desired compound.

### Method P: General Procedure for Stille Coupling

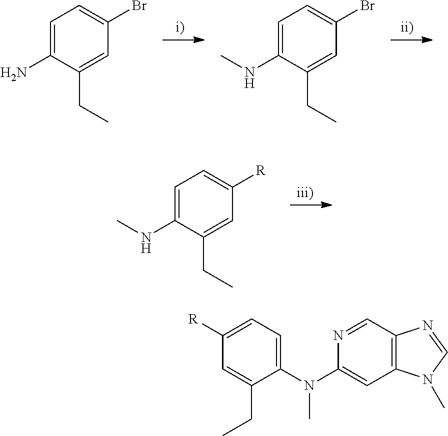

### Step i: 4-Bromo-2-ethyl-N-methylaniline

A mixture of 2-ethyl-4-bromoaniline (20 mmol), copper(II) acetate (50 mmol) and pyridine (70 mmol) in 1,4-dioxane (240 mL) are stirred at room temperature for 15 min. Methylboronic acid (50 mmol) is added and the resulting mixture is heated to reflux for 48 h. The mixture is cooled to room temperature, filtered through Celite and concentrated in vacuo. Purification by silica chromatography to give the desired compound.

### Step ii

A stirred mixture of 4-bromo-2-ethyl-N-methylaniline (2.42 mmol), the stannane (2.66 mmol), cesium fluoride (5.32 mmol) and tris(dibenzylideneacetone)dipalladium/tri-tert-butyl phosphonium tetrafluoroborate mixture (mole ratio: 1/1.2) (0.12 mmol) in 1,4-dioxane (5 mL) is degassed under N₂(g) for 10 min. The reaction mixture is heated to reflux for 24 h, cooled to room temperature and filtered through Celite. After concentrating in vacuo the resulting residue is dissolved in DMSO (1 mL) and purified by preparative HPLC.

### Step iii

At room temperature, 6-chloro-1-methyl-1H-imidazo[4,5-c]pyridine (0.80 mmol) is stirred with 2-dicyclohexylphosphino-2′,4′,6′-triisopropylbiphenyl (0.0365 mmol), chloro(2-dicyclohexylphosphino-2′,4′,6′-triisopropyl-1,1′-biphenyl)[2-(2-aminoethyl)phenyl]palladium(II) (0.0365 mmol), sodium tert-butoxide (1.10 mmol) and the aniline (0.73 mmol) in 1,4-dioxane (5 mL) and degassed under N₂ (g) for 10 min. At this point the reaction is stirred at 100° C. for 8 h. The mixture is cooled to room temperature and filtered through Celite and concentrated in vacuo. The resulting residue is dissolved in DMSO (1 mL) and purified by preparative HPLC to give the desired compound.

### Method Q: Methyl 4-ethyl-3-(methyl(1-methyl-1H-imidazo[4,5-c]pyridin-6-yl)amino)benzoate

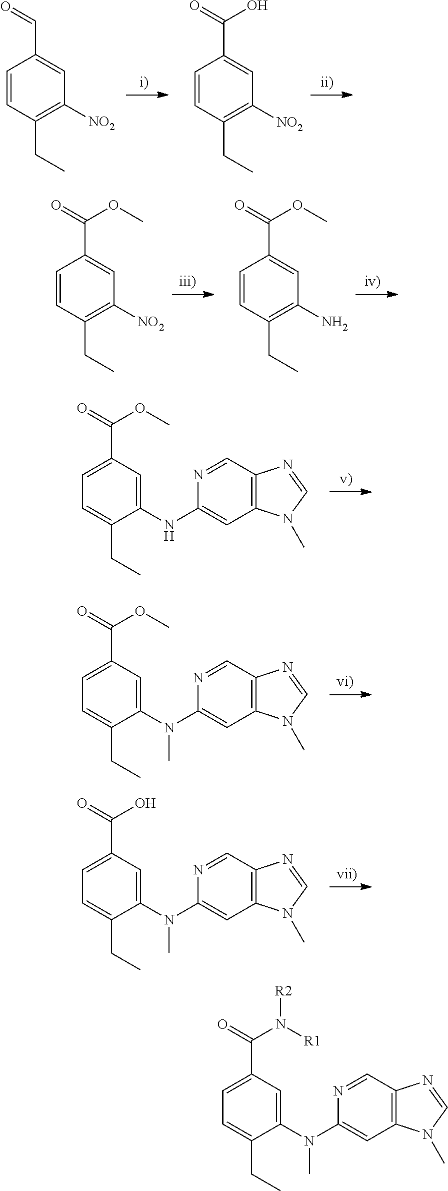

### Step i): 4-Ethyl-3-nitrobenzoic acid

4-Ethyl-3-nitrobenzaldehyde (0.05 mol) is added to a stirred solution of potassium permanganate (0.1 mol) and sodium hydroxide (0.0125 mol) in water (250 mL) at room temperature. The mixture is stirred under reflux for 2.5 h. The cooled mixture is filtered through Celite and the filtrate is acidified (pH 1) with 2M HCl (aqueous). The resulting suspension is extracted with DCM and the organics are dried (MgSO₄) and concentrated in vacuo to give the desired compound.

¹H NMR δ (ppm)(DMSO-d₆): 13.52 (1H, s, OH), 8.41-8.32 (1H, m, ArH), 8.25-8.14 (1H, m, ArH), 7.74-7.63 (1H, m, ArH), 2.89 (2H, q, CH2), 1.23 (3H, t, CH3).

### Step ii): Methyl 4-ethyl-3-nitrobenzoate

Acetyl chloride (1 mL) is added to MeOH (75 mL) and the resulting solution is stirred at room temperature for 10 min. 4-Ethyl-3-nitrobenzoic acid (4.73 g) is added and the resulting cloudy solution is refluxed for 6 h. The reaction mixture is concentrated in vacuo and the residue is dissolved in diethyl ether and washed with saturated aqueous sodium bicarbonate solution. The organics are concentrated in vacuo to give the desired compound.

¹H NMR δ (ppm)(CHCl₃-d): 8.57-8.50 (1H, m, ArH), 8.41-8.16 (1H, m, ArH), 7.49 (1H, dd, ArH), 4.18-3.77 (3H, s, CH3), 2.97 (2H, q, CH2), 1.32 (3H, t, CH3).

### Step iii): Methyl 3-amino-4-ethylbenzoate

Palladium-on-carbon (10% wt., 4.9 g) is added portion-wise to a stirred solution of ammonium formate (10.2 g) and methyl 4-ethyl-3-nitrobenzoate (0.021 mol) in MeOH (80 mL) at room temperature under N₂. The reaction mixture is stirred at gentle reflux for 1.5 h and cooled to room temperature. The reaction mixture is filtered through Celite and the filtrate is concentrated in vacuo. The residue is treated with water (20 mL) and extracted into ethyl acetate. The organics are dried (MgSO₄) and concentrated in vacuo to give the desired compound.

¹H NMR δ (ppm)(CHCl₃-d): 7.43-7.39 (1H, m, ArH), 7.27-7.26 (1H, d, ArH), 7.21-7.07 (1H, m, ArH), 4.09-3.68 (3H, m, CH3), 3.72 (2H, s, NH2), 2.62-2.50 (2H, m, CH2), 1.29-1.23 (3H, m, CH3).

### Step iv): Methyl 4-ethyl-3-(methyl(1-methyl-1H-imidazo[4,5-c]pyridin-6-yl)amino)benzoate

6-Chloro-1-methyl-1H-imidazo[4,5-c]pyridine (1.67 g, 0.01 mol), methyl 3-amino-4-ethylbenzoate (2.15 g, 0.012 mol) and cesium carbonate (4.56 g, 0.014 mol) are stirred together in tert-butanol (20 mL) under N2 for 20 min. Chloro[2-(dicyclohexylphosphino)-3,6-dimethoxy-2′-4′-6′-tri-1-propyl-1,1′-biphenyl][2-(2-aminoethyl)phenyl]palladium(II) (BrettPhos Palladacycle) (0.079 g, 1 mol %) and 2-dicyclohexylphosphino-2′,6′-di-iso-propoxy-1,1′-biphenyl (RuPhos) (0.046 g, 1 mol %) are added and the mixture is refluxed for 5 h. The reaction mixture is concentrated in vacuo and the residue is treated with water and extracted into EtOAc. The organics are dried (MgSO₄) and concentrated in vacuo to give an oily solid. The residue is dissolved in MeOH and loaded onto a 70 g SCX column which is washed with MeOH (150 mL) before eluting the product with 7 N NH₃ in MeOH:MeOH (1:4). The eluent is concentrated in vacuo and the residue is purified by silica gel flash chromatography (DCM to 5% MeOH in DCM) to give the desired compound.

¹H NMR δ (ppm)(DMSO-d₆): 8.51 (1H, s, ArH), 8.16 (1H, d, ArH), 8.07 (2H, d, ArH, NH), 7.58 (1H, dd, ArH), 7.35 (1H, d, ArH), 6.83 (1H, s, ArH), 3.96-3.72 (3H, m, CH3), 3.72 (3H, s, CH3), 2.71 (2H, q, CH2), 1.17 (3H, t, CH3).

LCMS (10 cm_ESCI_Bicarb_MeCN) Rt 2.77 (min) m/z 311 (MH⁺).

### Step v): Methyl 4-ethyl-3-(methyl(1-methyl-1H-imidazo[4,5-c]pyridin-6-yl)amino)benzoate

Sodium hydride (60% in oil, 0.188 g, 4.7 mmol) is added to a stirred solution of methyl 4-ethyl-3-(methyl(1-methyl-1H-imidazo[4,5-c]pyridin-6-yl)amino)benzoate (1.33 g, 4.3 mmol) in dry DMF (30 mL) under nitrogen. The mixture is stirred for 10 min at room temperature before the addition of iodomethane (0.67 g, 4.7 mmol) and stirring is continued at room temperature for 3 h. The solvent is concentrated in vacuo and the residue was treated with dilute citric acid and extracted into ethyl acetate. The organics are dried (MgSO₄), filtered and concentrated in vacuo to give the desired compound, which is used in the next step without further purification.

### Step vi): 4-Ethyl-3-(methyl(1-methyl-1H-imidazo[4,5-c]pyridin-6-yl)amino)benzoic acid

The crude methyl 4-ethyl-3-(methyl(1-methyl-1H-imidazo[4,5-c]pyridin-6-yl)amino)benzoate (1.5 g) is added to a solution of sodium hydroxide in water (60 mL) and MeOH (20 mL) and the resulting suspension is stirred for 4.5 h at room temperature to give a cloudy solution. The mixture is washed with ethyl acetate and concentrated in vacuo to give an oil which is dissolved in water and acidified with 2 M HCl (aqueous). The resulting oil is extracted into DCM, dried (MgSO₄), filtered and concentrated in vacuo to give the desired compound which is used in the next step without further purification.

### Step vii): General Method for Amide Coupling

(2-(7-Aza-1H-benzotriazole-1-yl)-1,1,3,3-tetramethyluronium hexafluorophosphate) (0.084 g, 0.22 mmol) is added to a stirred solution 4-ethyl-3-(methyl(1-methyl-1H-imidazo[4,5-c]pyridin-6-yl)amino)benzoic acid (0.062 g, 0.2 mmol) and triethylamine (0.06 g, 0.6 mmol) in DMF (1 mL) and the resulting solution is stirred at room temperature for 30 min. The amine (0.4 mmol) is added and the reaction is stirred at room temperature for 1 d. The reaction mixture is filtered and the filtrate is purified by preparative HPLC to give the desired compound.

### Method R: General Procedure for Triazole Formation

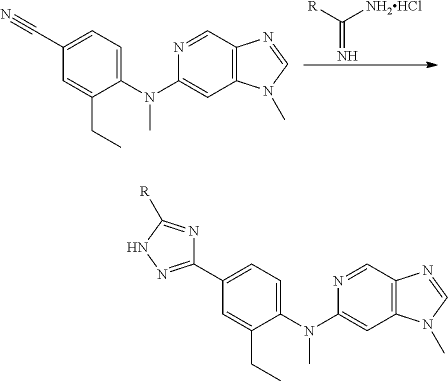

3-Ethyl-4-(methyl(1-methyl-1H-imidazo[4,5-c]pyridin-6-yl)amino)benzonitrile (0.69 mmol), the amidine hydrochloride (1.04 mmol), copper(I) bromide (0.035 mmol) and cesium carbonate (2.07 mmol) in DMSO (6 mL) are heated to 120° C. and stirred vigorously in the presence of air for 1 d. The reaction mixture is cooled to room temperature, filtered and the filtrate is submitted for purification by preparative HPLC to give the desired compound.

### Method S: General Procedure for Urea Formation

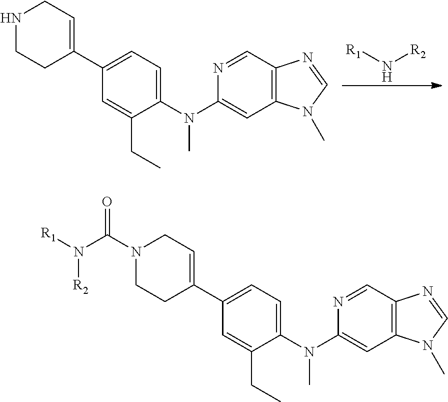

The amine (0.32 mmol) and triethylamine (45 μL, 0.32 mmol) are stirred in DCM (2 mL) at room temperature. Bis(trichloromethyl)carbonate (32 mg, 0.11 mmol) is added and the mixture stirred at room temperature for 1 h. Triethylamine (89 μL, 0.64 mmol) is added followed by a solution of the amine scaffold (0.32 mmol) in DCM (2 mL) and the reaction mixture is stirred at room temperature for between 1 h and 1 d. DCM and water are added and the mixture was filtered through a hydrophobic frit. The filtrate is concentrated in vacuo and purified by preparative HPLC to give the desired compound.

### Method T: General Procedure for Sulfonylation

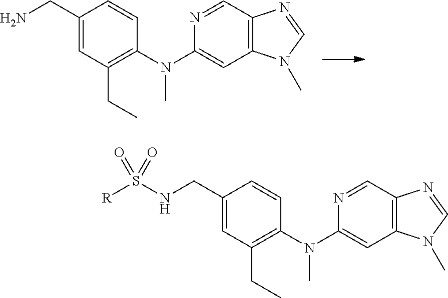

The sulfonyl chloride (0.22 mmol) is added to a solution of N-(2-ethyl-4-((2-methyl-1H-imidazol-1-yl)methyl)phenyl)-N,1-dimethyl-1H-imidazo[4,5-c]pyridin-6-amine (60 mg, 0.203 mmol) and triethylamine (85 μL, 0.61 mmol) in DCM (2 mL). The reaction mixture is stirred at room temperature for 1 d and concentrated in vacuo. The residue is purified by preparative HPLC to give the desired compound.

### Method U: General Procedure for Sulfonylation

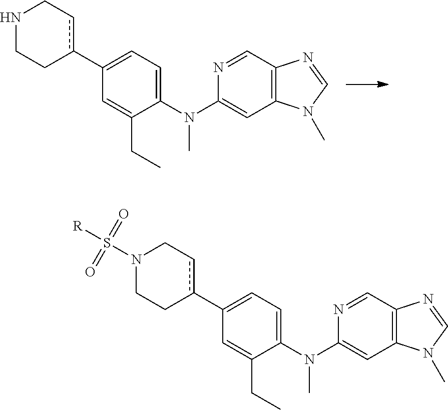

The sulfonyl chloride (0.46 mmol) is added to a solution of the amine scaffold (0.22 mmol) in DCM (1.5 mL) at 0° C. and stirred for 10 min. Triethylamine (47 μL, 0.46 mmol) is added and the reaction is stirred at room temperature for 1 d. The reaction mixture is concentrated in vacuo and the resulting residue is purified by preparative HPLC.

### Method V: General Procedure for Urea Formation

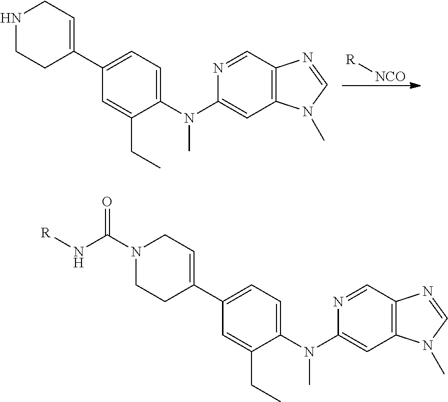

The isocyanate (0.34 mmol) is added to a solution of the amine (0.23 mmol) and triethylamine (27 μL, 0.34 mmol) in DCM (1.8 mL) at room temperature. The reaction mixture is stirred at room temperature for 2 h and concentrated in vacuo. The resulting residue is purified by preparative HPLC to give the desired compound.

### Method W: General Procedure for Displacement with Alcohols

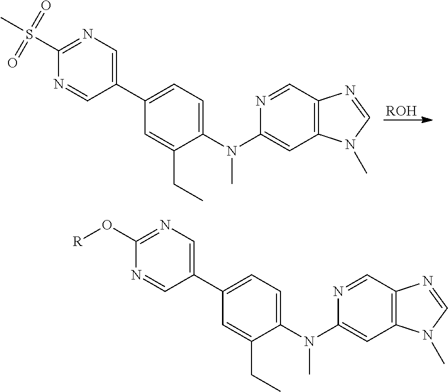

N-(2-Ethyl-4-(2-(methylsulfonyl)pyrimidin-5-yl)phenyl)-N,1-dimethyl-1H-imidazo[4,5-c]pyridin-6-amine (100 mg, 0.24 mmol), the alcohol (0.35 mmol) and potassium tert-butoxide (93 mg, 0.83 mmol) are heated at reflux in THF (4 mL) for 1 d. DCM and water are added and the layers separated with a hydrophobic frit. The filtrated is concentrated in vacuo and the residue is purified by preparative HPLC.

### Method X: General Procedure for Substituted Pyrazoles

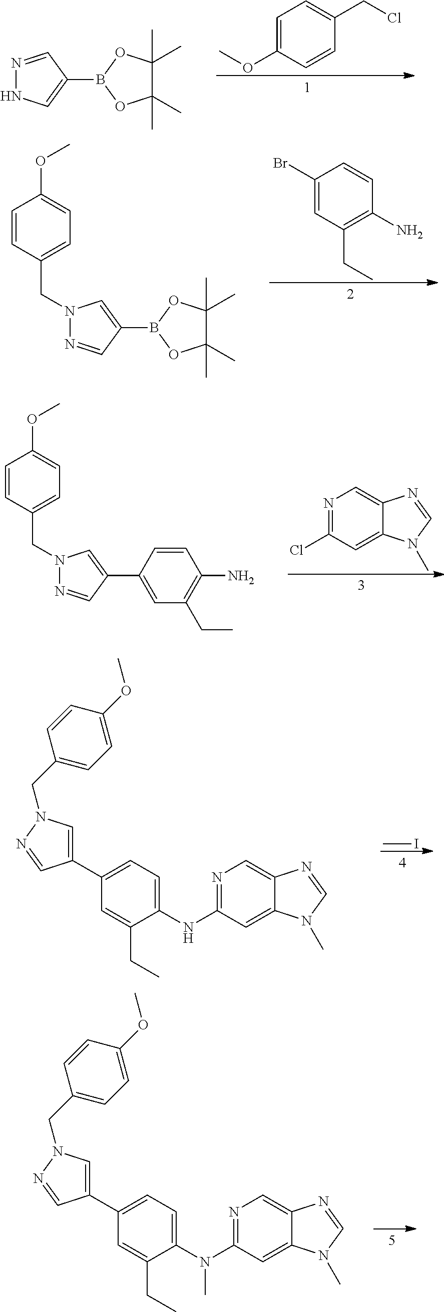

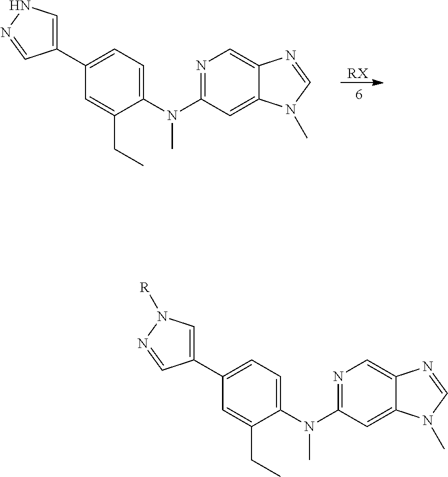

### Step 1: 1-(4-Methoxybenzyl)-4-(4,4,5,5-tetramethyl-1,3,2-dioxaborolan-2-yl)-1H-pyrazole

4-Methoxybenzyl chloride (0.0257 M) is added to a stirred suspension of 4-(4,4,5,5-tetramethyl-1,3,2-dioxaborolan-2-yl)-1H-pyrazole (0.0257 M) and cesium carbonate (0.0282 M) in acetonitrile (30 mL) and the resulting suspension is stirred at reflux for 4 h. The cooled reaction mixture is poured onto water and extracted into DCM, the combined extracts are dried (MgSO₄) and concentrated in vacuo to give the desired compound.

¹H NMR δ (ppm)(CHCl₃-d): 7.80 (1H, s, ArH), 7.66-7.59 (1H, m, ArH), 7.24-7.16 (2H, m, ArH), 6.90-6.83 (2H, m, ArH), 5.29-5.19 (2H, m, CH), 3.85-3.74 (3H, m, CH), 1.52-1.09 (12H, m, CH).

### Step 2: 2-Ethyl-4-(1-(4-methoxybenzyl)-1H-pyrazol-4-yl)aniline

A suspension of 1-(4-methoxybenzyl)-4-(4,4,5,5-tetramethyl-1,3,2-dioxaborolan-2-yl)-1H-pyrazole (0.0247 M), 4-bromo-2-ethylaniline (0.0247 M), cesium carbonate (0.0494 M) and [1,1′-bis(diphenylphosphino)ferrocene]dichloropalladium(II) (2.01 g, 10 mol %) is stirred at 100° C. under N₂ for 6.5 h. The reaction mixture is concentrated in vacuo and the residue is treated with DCM (400 mL) and filtered through Celite. The filtrate is concentrated in vacuo and the residue was purified by silica gel flash chromatography (10-30% EtOAc in iso-hexanes) to give the desired compound.

¹H NMR δ (ppm)(CHCl₃-d): 7.71 (1H, d, ArH), 7.48 (1H, d, ArH), 7.29-7.07 (4H, m, ArH), 6.90-6.85 (2H, m, ArH), 6.66 (1H, d, ArH), 5.28-5.21 (2H, m, CH2), 3.80 (3H, s, CH3), 3.61 (2H, s, NH), 2.53 (2H, q, CH2), 1.30-1.20 (3H, m, CH3).

### Step 3: N-(2-Ethyl-4-(1-(4-methoxybenzyl)-1H-pyrazol-4-yl)phenyl)-1-methyl-M-imidazo[4,5-c]pyridin-6-amine

A solution of 2-ethyl-4-(1-(4-methoxybenzyl)-1H-pyrazol-4-yl)aniline (0.013 M), 6-chloro-1-methyl-1H-imidazo[4,5-c]pyridine (0.013 M), 4,5-bis(diphenylphosphino)-9,9-dimethylxanthene (10 mol %) and sodium tert-butoxide (0.0195 M) in dry 1,4-dioxane (100 mL) is stirred under N2 for 15 min. Tris(dibenzylideneacetone)dipalladium(0) (5 mol %) is added and the reaction is stirred at 105° C. for 1.5 h. The mixture is concentrated in vacuo and the residue is dissolved in DCM and filtered through Celite. The filtrate is washed with water, dried MgSO₄ and concentrated in vacuo. The residue is purified by silica gel flash chromatography (1-5% MeOH in DCM) to give the desired compound.

### Step 4: N-(2-Ethyl-4-(1-(4-methoxybenzyl)-1H-pyrazol-4-yl)phenyl)-N,1-dimethyl-1H-imidazo[4,5-c]pyridin-6-amine

Sodium bis(trimethylsilyl)amide (1 M in THF, 0.0111 M, 11.1 mL) is added to a stirred solution of N-(2-ethyl-4-(1-(4-methoxybenzyl)-1H-pyrazol-4-yl)phenyl)-1-methyl-1H-imidazo[4,5-c]pyridin-6-amine (0.0111 M) in dry DMF (50 mL) under N2 and the resulting solution is stirred at room temperature for 20 min. Iodomethane (0.0111 M) dissolved in dry DMF (4 mL) is added dropwise and the resulting solution is stirred for 1.5 h at room temperature. The solvent is evaporated in vacuo and the residue is treated with DCM and washed with water. The organics are dried MgSO₄, filtered and concentrated in vacuo, the residue is purified by silica gel flash chromatography (DCM to 2% MeOH in DCM) to give the desired compound.

¹H NMR δ (ppm)(DMSO-d₆): 8.50 (1H, d, ArH), 8.28 (1H, s, ArH), 8.01-7.88 (2H, m, ArH), 7.63-7.57 (1H, m, ArH), 7.49 (1H, dd, ArH), 7.30-7.22 (2H, m, ArH), 7.15 (1H, d, ArH), 6.95-6.90 (2H, m, ArH), 6.11 (1H, s, ArH), 5.28 (2H, s, CH2), 3.74 (3H, s, CH3), 3.57 (3H, s, CH3), 3.34 (3H, d, CH3), 2.49-2.42 (2H, m, CH2), 1.12 (3H, t, CH3).

LCMS (10 cm_ESCI_Formic_MeCN) Rt 2.81 (min) m/z 453 (MH⁺).

### Step 5: N-(2-Ethyl-4-(1H-pyrazol-4-yl)phenyl)-N,1-dimethyl-1H-imidazo[4,5-c]pyridin-6-amine

A solution of N-(2-ethyl-4-(1-(4-methoxybenzyl)-1H-pyrazol-4-yl)phenyl)-N,1-dimethyl-1H-imidazo[4,5-c]pyridin-6-amine (2.2 mmol) in trifluoroacetic acid (15 mL) is allowed to stand at room temperature for 3 d. The solution is then heated to 130° C. using microwave irradiation for 10 min. The solution is concentrated in vacuo and the residue was dissolved in DCM and washed with saturated aqueous sodium bicarbonate solution. The organics are dried (MgSO₄), filtered and concentrated in vacuo. The residue is purified by silica gel flash chromatography (DCM to 4% MeOH in DCM) to give the desired compound.

¹H NMR δ (ppm)(DMSO-d₆): 12.95 (1H, s, NH), 8.50 (1H, d, ArH), 8.22 (1H, s, ArH), 7.97 (2H, s, ArH), 7.63 (1H, d, ArH), 7.53 (1H, dd, ArH), 7.16 (1H, d, ArH), 6.13 (1H, d, ArH), 3.58 (3H, s, CH3), 3.34 (3H, d, CH3), 2.54-2.43 (2H, m, CH2), 1.13 (3H, t, CH3).

LCMS (10 cm_ESCI_Bicarb_MeCN) Rt 2.53 (min) m/z 333 (MH⁺).

### Step 6: General Procedure for the Alkylation of N-(2-ethyl-4-(1H-pyrazol-4-yl)phenyl)-N,1-dimethyl-1H-imidazo[4,5-c]pyridin-6-amine

Sodium bis(trimethylsilyl)amide (1 M in THF, 0.33 mL, 0.33 mmol) is added to a solution of N-(2-ethyl-4-(1H-pyrazol-4-yl)phenyl)-N,1-dimethyl-1H-imidazo[4,5-c]pyridin-6-amine (99 mg, 0.3 mmol) in DMF (0.7 mL) and the resulting solution is stirred at room temperature for 15 min. The alkyl halide (0.3 mmol) is added and stirring was continued for 5 h. The resulting mixture is filtered and the filtrate purified by preparative HPLC to give the desired compound.

### Method Y: General Procedure for Amide Formation

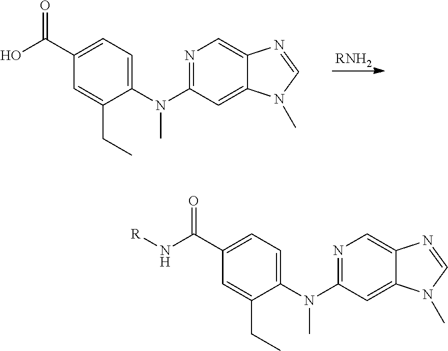

(2-(7-Aza-1H-benzotriazole-1-yl)-1,1,3,3-tetramethyluronium hexaflurophosphate) (0.19 g, 0.5 mmol) was added to a stirred suspension of 3-ethyl-4-(methyl(1-methyl-1H-imidazo[4,5-c]pyridin-6-yl)amino)benzoic acid (0.124 g, 0.4 mmol) and N-ethyl-N-isopropylpropan-2-amine (0.15 g, 1.2 mmol) in DMF (3 mL) at room temperature and the resulting solution is stirred at room temperature for 10 min. The amine (0.6 mmol) is added and stirring was continued for 20 h. The crude reaction mixture is purified by preparative HPLC to give the desired compound.

### Method Z: General Procedure for Amide Coupling

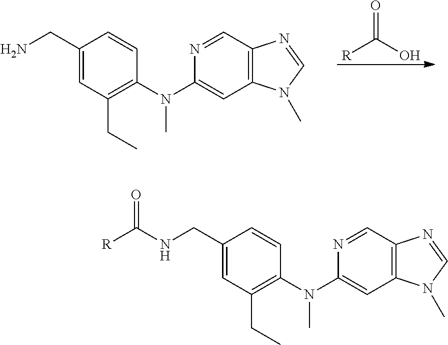

The acid (1.1 equiv.), 2-(7-aza-1H-benzotriazole-1-yl)-1,1,3,3-tetramethyluronium hexafluorophosphate (1.2 equiv.) and diisopropylethylamine (2.5 equiv.) were stirred in DMF at room temperature for 30 min. Then N-(4-(aminomethyl)-2-ethylphenyl)-N,1-dimethyl-1H-imidazo[4,5-c]pyridin-6-amine (1 equiv.) in DMF was added and the reaction mixture stirred at room temperature for 8 h. The crude reaction mixture was purified by preparative HPLC to give the desired compound.

### Method AA: General Procedure for Urea Formation from Carbamate

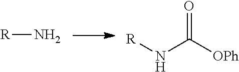

Phenyl chloroformate (8.1 mmol) is added slowly to a mixture of the amine (1 equiv.) and 4-dimethylaminopyridine (0.41 mmol) in pyridine (10 mL) at 0° C. The reaction mixture is stirred at room temperature for 3 h and poured into ice water. The resulting precipitate is filtered, washed with water and dried in vacuo to give the carbamate intermediate.

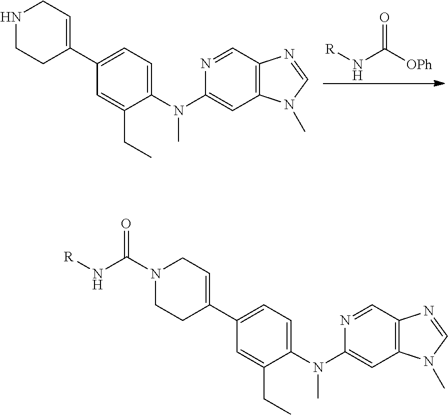

A solution of N-(2-ethyl-4-(1,2,3,6-tetrahydropyridin-4-yl)phenyl)-N,1-dimethyl-1H-imidazo[4,5-c]pyridin-6-amine (0.1 g, 0.29 mmol), the carbamate (0.29 mmol) and diisopropylethylamine (51 μL, 0.29 mmol) in DMF (3 mL) is stirred at room temperature for 1 d. The crude reaction mixture is purified by preparative HPLC.

### Representative Examples for the Preparation of the Compounds of Invention

### Compound 7 and Compound 13: (4-(ethyl(1-methyl-1H-imidazo[4,5-c]pyridin-6-yl)amino)phenyl)MeOH

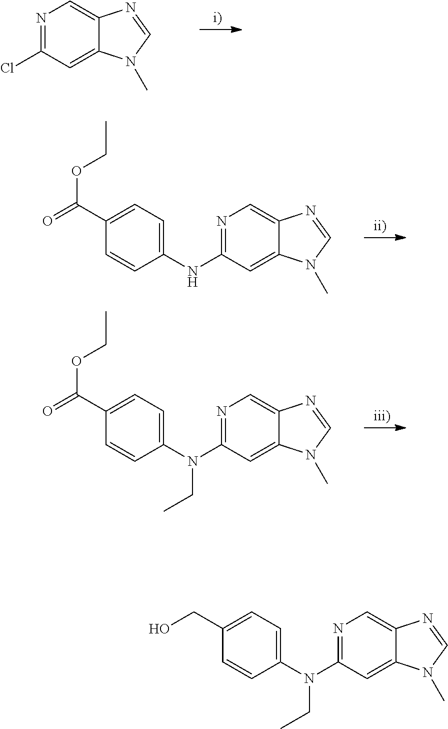 [SMILES: Cn1cnc2cnc(Cl)cc21 ; CCOC(=O)c1ccc(Nc2cc3c(cn2)ncn3C)cc1 ; CCOC(=O)c1ccc(N(CC)c2cc3c(cn2)ncn3C)cc1 ; CCN(c1ccc(CO)cc1)c1cc2c(cn1)ncn2C]

### Step (i): 4-(1-Methyl-1H-imidazo[4,5-c]pyridin-6-ylamino)-benzoic acid ethyl ester

Intermediate 2 and 4-aminobenzoic acid ethyl ester are reacted according to method A′

### Step (ii): 4-[Ethyl-(1-methyl-1H-imidazo[4,5-c]pyridin-6-yl)-amino]-benzoic acid ethyl ester

The product obtained in step i) above is reacted as described in method C.

### Step (iii): {4-[Ethyl-(1-methyl-1H-imidazo[4,5-c]pyridin-6-yl)-amino]-phenyl}-MeOH

To a solution of LiAlH₄ (1M in THF, 0.23 mL) in THF (1 mL) was added at 0° C. a solution of 4-[Ethyl-(1-methyl-1H-imidazo[4,5-c]pyridin-6-yl)-amino]-benzoic acid ethyl ester (34 mg, 0.1 mmol) in THF (2 mL). The mixture was stirred for 1 h, diluted with AcOEt and washed with an aqueous saturated solution of NH4Cl. The organic phase was dried over MgSO₄, concentrated and purified on silica gel (DCM/MeOH: 100/0 to 100/5) to give the {4-[Ethyl-(1-methyl-1H-imidazo[4,5-c]pyridin-6-yl)-amino]-phenyl}-MeOH.

### Compound 16: (2-Ethyl-phenyl)-methyl-(1-methyl-1H-imidazo[4,5-c]pyridin-6-yl)-amine

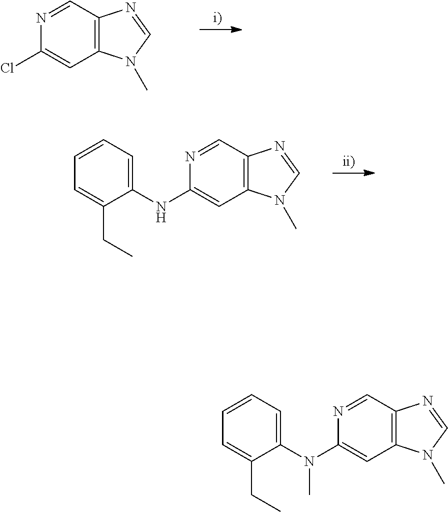 [SMILES: Cn1cnc2cnc(Cl)cc21 ; CCc1ccccc1Nc1cc2c(cn1)ncn2C ; CCc1ccccc1N(C)c1cc2c(cn1)ncn2C]

### Step i): (2-Ethyl-phenyl)-(1-methyl-1H-imidazo[4,5-c]pyridin-6-yl)-amine

In a microwave vial was introduced Intermediate 2 (150 mg, 0.895 mmol), the 2-ethyl aniline (0.14 mL, 1.163 mmol) and Cs₂CO₃ (408.2 mg, 1.253 mmol) in 1,4-dioxane (4 mL). The reaction mixture was degassed with N₂ during 10 min. A mixture of Pd₂(dba)₃ (24.6 mg, 0.027 mmol) and xantphos (31.1 mg, 0.054 mmol) in 1,4-dioxane (2 mL) was sonicated and then added under N₂ to the reaction mixture. The reaction mixture was degassed with N₂ for another 10 min. The vial was capped and the mixture was stirred at 110° C. overnight. The reaction mixture was concentrated in vacuo, the crude was then purified by flash column chromatography on silica gel using DCM/MeOH (gradient 100 to 95/5) afforded the (2-Ethyl-phenyl)-(1-methyl-1H-imidazo[4,5-c]pyridin-6-yl)-amine.

LC-MS: [M+H]+: 253.33.

### Step ii): (2-Ethyl-phenyl)-methyl-(1-methyl-1H-imidazo[4,5-c]pyridin-6-yl)-amine

To a suspension of sodium hydride (60% in oil, 1.25 equiv) in DMF (0.5 mL) was added dropwise the (2-Ethyl-phenyl)-(1-methyl-1H-imidazo[4,5-c]pyridin-6-yl)-amine (0.238 mmol, 1 equiv) previously dissolved in DMF (0.5 mL). The reaction was stirred at room temperature for 20 min, then methyl iodide (1.1 equiv) was added dropwise. The reaction mixture was stirred at room temperature for 2 h, then was diluted with EtOAc and washed with water. The organic layer was dried over sodium sulphate, filtered, and concentrated in vacuo. The crude was then purified by flash column chromatography on silica gel using DCM/MeOH (gradient 100 to 95/5) afforded (2-Ethyl-phenyl)-methyl-(1-methyl-1H-imidazo[4,5-c]pyridin-6-yl)-amine (Compound 16).

LC-MS: [M+H]+: 267.38

### Compound 17: 4-(methyl(1-methyl-1H-imidazo[4,5-c]pyridin-6-yl)amino)phenol

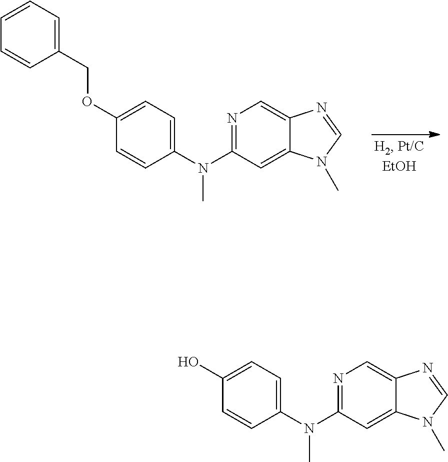 [SMILES: CN(c1ccc(OCc2ccccc2)cc1)c1cc2c(cn1)ncn2C ; CN(c1ccc(O)cc1)c1cc2c(cn1)ncn2C]

To a solution of (4-Benzyloxy-phenyl)-methyl-(1-methyl-1H-imidazo[4,5-c]pyridin-6-yl)-amine (compound 25) (1.8 g, 5.2 mmol) in EtOH (50 mL) was added Pt/C (10%, 150 mg) and the reaction was conducted under hydrogen. After 24 h, the mixture was filtered on clarcel, evaporated and purified on silica gel (DCM/MeOH:95/5) to give the desired compound.

### Compound 26: 2-(4-(Methyl(1-methyl-1H-imidazo[4,5-c]pyridin-6-yl)amino)phenyl)acetonitrile

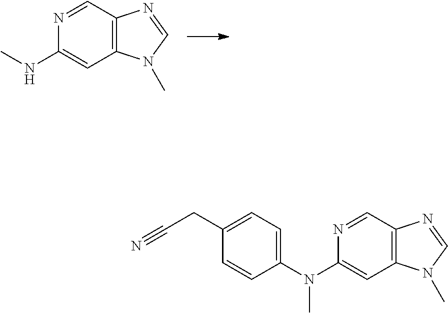 [SMILES: CNc1cc2c(cn1)ncn2C ; CN(c1ccc(CC#N)cc1)c1cc2c(cn1)ncn2C]

A mixture of intermediate 5 (100 mg, 0.61 mmol), 4-bromophenylacetonitrile (127 mg, 0.64 mmol), dibenzylideneacetone bis(triphenylphiosphine) palladium (0) (16 mg, 0.017 mmol), 4,5-bis(diphenylphosphino)-9,9-dimethylxanthene (20 mg, 0.035 mmol), cesium carbonate (295 mg, 0.91 mmol) and dioxane (3 mL) was stirred at 100° C. for 16 h. The reaction mixture was allowed to cool to room temperature and the solvent removed in vacuo. Sat. NaCl (aq.) (5 mL) was added to the residue and the mixture was extracted with EtOAc (4×3 mL). The combined organic extracts were dried (MgSO₄), filtered and the solvent removed in vacuo. The crude product was purified by preparative HPLC to give the desired compound.

¹H NMR δ (ppm)(CHCl₃-d): 8.78 (1H, s, ArH), 7.72 (1H, s, ArH), 7.22-7.34 (4H, m, ArH), 6.61 (1H, s, ArH), 3.75 (2H, s, CH2), 3.68 (3H, s, CH3), 3.53 (3H, s, CH3).

LCMS (10 cm_ESI_Bicarb_MeCN) Rt 2.41 (min) m/z 278 (MH⁺).

### Compound 27: Methyl 3-methyl-4-(methyl(1-methyl-1H-imidazo[4,5-c]pyridin-6-yl)amino)benzoate; and

### Compound 29: (3-Methyl-4-(methyl(1-methyl-1H-imidazo[4,5-c]pyridin-6-yl)amino)phenyl) MeOH

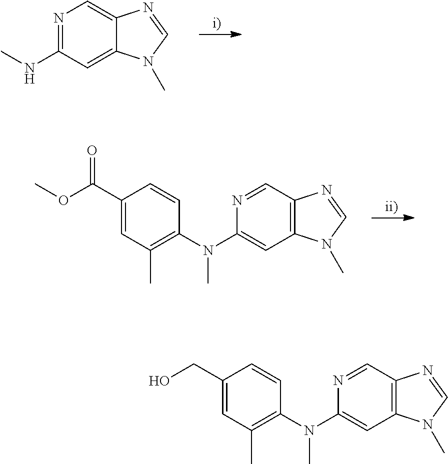 [SMILES: CNc1cc2c(cn1)ncn2C ; COC(=O)c1ccc(N(C)c2cc3c(cn2)ncn3C)c(C)c1 ; Cc1cc(CO)ccc1N(C)c1cc2c(cn1)ncn2C]

### Step i): Methyl 3-methyl-4-(methyl(1-methyl-1H-imidazo[4,5-c]pyridin-6-yl)amino)benzoate (Compound 27)

A mixture of N,1-dimethyl-1H-imidazo[4,5-c]pyridin-6-amine (1.5 g, 9.3 mmol), methyl 4-bromo-3-methylbenzoate (1.9 g 8.5 mmol), dibenzylideneacetone bis(triphenylphiosphine) palladium (0) (0.38 g, 0.415 mmol), 4,5-bis(diphenylphosphino)-9,9-dimethylxanthene (0.49 g, 0.85 mmol), sodium tert-butoxide (1.21 g, 12.6 mmol) and dioxane (20 mL) was stirred at 90° C. for 16 h. The reaction mixture was allowed to cool to room temperature and concentrated in vacuo. The residue was dissolved in DCM (100 mL) and water (50 mL). The organic phase was separated and the aqueous phase was extracted with DCM (100 mL). The combined organic extracts were dried (MgSO₄), filtered and the solvent removed in vacuo. The crude product was purified by column chromatography using silica gel and eluting with 0-10% MeOH in DCM to give a brown solid (1.3 g). A sample (100 mg) of this material was further purified by preparative HPLC to give the desired compound.

¹H NMR δ (ppm)(CHCl₃-d): 8.74 (1H, s, ArH), 8.02 (1H, m, ArH), 7.96 (1H, m, ArH), 7.65 (1H, s, ArH), 7.30 (1H, d, ArH), 5.99 (1H, s, ArH), 3.94 (3H, s, CH3), 3.58 (3H, s, CH3), 3.48 (3H, s, CH3), 2.18 (3H, s, CH3).

LCMS (10 cm_ESCI_Formic_MeCN) Rt 2.41 (min) m/z 311 (MH⁺).

### Step ii): (3-Methyl-4-(methyl(1-methyl-1H-imidazo[4,5-c]pyridin-6-yl)amino)phenyl) MeOH (Compound 29)

Methyl 3-methyl-4-(methyl(1-methyl-1H-imidazo[4,5-c]pyridin-6-yl)amino)benzoate (480 mg, 1.55 mmol) was dissolved in THF (10 mL) and cooled to 0° C. under N₂ (g). Lithium aluminum hydride (1.5 mL, 1.55 mmol, 1 M solution in THF) was added slowly and the reaction mixture was allowed to warm to room temperature over 1 h. Concentrated NaOH solution (aq. 3 mL) was added slowly followed by water (3 mL). The reaction was extracted with EtOAc and the organic phase was washed with water and brine then dried (MgSO₄), filtered and concentrated in vacuo. The desired compound was used in the subsequent steps without further purification. A sample was purified by preparative HPLC to give the desired compound.

¹H NMR δ (ppm)(DMSO-d₆): 8.49 (1H, s, ArH), 7.97 (1H, s, ArH), 7.29 (1H, s, ArH), 7.22 (1H, d, ArH), 7.15 (1H, d, ArH), 6.10 (1H, s, ArH), 5.19 (1H, t, OH), 4.52 (2H, d, CH2), 3.58 (3H, s, CH3), 3.31 (3H, s, CH3), 2.07 (3H, s, CH3).

LCMS (10 cm_ESCI_Bicarb_MeCN) Rt 2.30 (min) m/z 283 (MH⁺).

### Compound 28: N-(2-Ethyl-4-fluorophenyl)-N,1-dimethyl-1H-imidazo[4,5-c]pyridin-6-amine

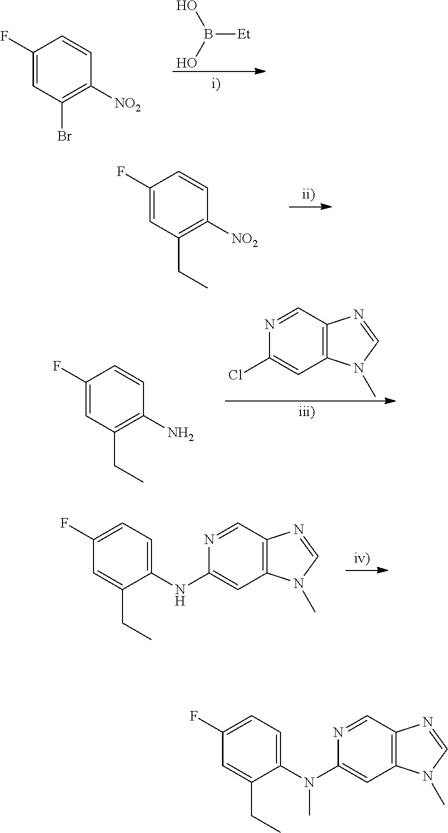

### Step i): 2-Ethyl-4-fluoro-1-nitrobenzene

2-Bromo-4-fluoro-1-nitrobenzene (1.63 g, 7.4 mmol), ethylboronic acid (0.6 g, 8.14 mmol), potassium carbonate (3.1 g, 22.2 mmol) and [1,1′-bis(diphenylphosphino)ferrocene]dichloropalladium(II) (302 mg, 0.37 mmol) were suspended in dioxane (25 mL) and water (5 mL). The stirred reaction mixture was degassed (N₂) and heated to 100° C. for 1 d. The reaction mixture was filtered through Celite and partitioned between EtOAc and water. The layers were separated and the aqueous layer was extracted with EtOAc, the organics were combined, dried (MgSO₄) and concentrated in vacuo. The resulting residue was purified by column chromatography using silica gel and eluting with 0-50% EtOAc in isohexanes to give the desired compound.

### Step ii): 2-Ethyl-4-fluoroaniline

2-Ethyl-4-fluoro-1-nitrobenzene (460 mg, 2.7 mmol), NH₄Cl (217 mg, 4.05 mmol) and iron powder (753 mg, 13.5 mmol) were suspended in THF (3 mL), ethanol (3 mL) and water (1 mL). The stirred reaction mixture was heated to 90° C. for 1 h. The reaction mixture was filtered through Celite, washed through with DCM and concentrated in vacuo. The resulting residue was partitioned between DCM and sat. aq. NaHCO₃ solution. The layers were separated and the aqueous layer extracted with DCM, the organics were combined, filtered through a hydrophobic filter and then concentrated in vacuo. The resulting residue was purified using column chromatography on silica gel and eluting with 0-25% EtOAc in isohexanes to give the desired compound.

¹H NMR δ (ppm)(DMSO-d₆): 6.77-6.67 (2H, m, ArH), 6.64-6.53 (1H, m, ArH), 4.69 (2H, s, NH2), 2.47-2.37 (2H, m, CH2), 1.19-1.07 (3H, m, CH3).

### Step iii): N-(2-Ethyl-4-fluorophenyl)-1-methyl-1H-imidazo[4,5-c]pyridin-6-amine

To stirred degassed (N₂) 1,4-dioxane (3 mL) was added 6-chloro-1-methyl-1H-imidazo[4,5-c]pyridine (219 mg, 1.31 mmol), 2-ethyl-4-fluoroaniline (200 mg, 1.44 mmol), sodium-tert-butoxide (189 mg, 1.97 mmol), tris(dibenzylideneacetone)dipalladium(0) (60 mg, 0.066 mmol) and 4,5-bis(diphenylphosphino)-9,9-dimethylxanthene (76 mg, 0.131 mmol). The reaction mixture was heated to 90° C. for 1 d. The reaction mixture was filtered through Celite, washed through with EtOAc and partitioned between EtOAc and water. The layers were separated and the aqueous layer extracted with EtOAc, the organics were combined and dried (MgSO₄), filtered and concentrated in vacuo to give the desired compound, which was used in the next step without further purification. A 100 mg sample was purified by preparative HPLC to give the desired compound.

¹H NMR δ (ppm)(DMSO-d₆): 8.45 (1H, s, NH), 8.01 (1H, s, ArH), 7.87 (1H, s, ArH), 7.46-7.39 (1H, m, ArH), 7.10-7.05 (1H, m, ArH), 7.03-6.96 (1H, m, ArH), 6.55 (1H, d, ArH), 3.67 (3H, s, CH3), 2.67-2.57 (2H, m, CH2), 1.16-1.07 (3H, m, CH3).

LCMS (10 cm_ESCI_Bicarb_MeCN) Rt 2.80 (min) m/z 271 (MH⁺).

### Step iv): N-(2-Ethyl-4-fluorophenyl)-N,1-dimethyl-1H-imidazo[4,5-c]pyridin-6-amine

A solution of N-(2-ethyl-4-fluorophenyl)-1-methyl-1H-imidazo[4,5-c]pyridin-6-amine (210 mg, 0.78 mmol) in DMF (3 mL) was added dropwise to a suspension of sodium hydride (60% dispersion in mineral oil, 38 mg, 0.936 mmol) in DMF (1 mL) and stirred at room temperature for 1 h. Iodomethane (80 μL, 1.56 mmol) was added and the reaction mixture was stirred for a further 1 h at room temperature. The reaction mixture was partitioned between DCM and water, the layers were separated and the aqueous layer extracted with DCM. The organics were combined, filtered through a hydrophobic filter then concentrated in vacuo. The resulting residue was purified by preparative HPLC to give the desired compound.

¹H NMR δ (ppm)(CHCl₃-d): 8.72 (1H, d, ArH), 7.61 (1H, s, ArH), 7.20-7.13 (1H, m, ArH), 7.11-7.06 (1H, m, ArH), 7.02-6.95 (1H, m, ArH), 5.85 (1H, d, ArH), 3.57 (3H, s, CH3), 3.43 (3H, s, CH3), 2.57-2.47 (2H, m, CH2), 1.21-1.12 (3H, m, CH3).

LCMS (15 cm_Bicarb_GeminiNX_HPLC_MeCN) Rt 9.65 (min) m/z 285 (MH⁺).

Compound 30: N,3-Dimethyl-4-(methyl(1-methyl-1H-imidazo[4,5-c]pyridin-6-yl)amino)-benzamide

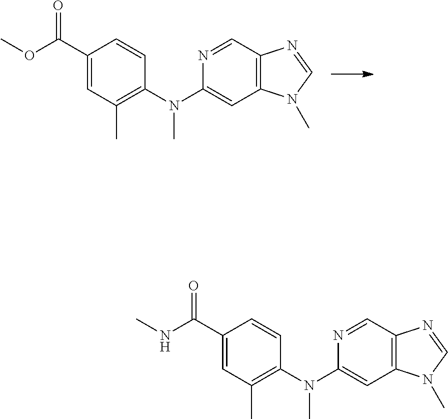 [SMILES: COC(=O)c1ccc(N(C)c2cc3c(cn2)ncn3C)c(C)c1 ; CNC(=O)c1ccc(N(C)c2cc3c(cn2)ncn3C)c(C)c1]

Methyl 3-methyl-4-(methyl(1-methyl-1H-imidazo[4,5-c]pyridin-6-yl)amino)benzoate (62 mg, 0.2 mmol) was weighed into a microwave tube and dissolved in a 33 wt % solution of MeNH₂ in EtOH (2 mL). The reaction vessel was capped and heated to 150° C. with microwave irradiation for 1 h. The reaction mixture was concentrated in vacuo and purified by preparative HPLC to give the desired compound.

¹H NMR δ (ppm)(CHCl₃-d): 8.72 (1H, d, ArH), 7.76 (1H, d, ArH), 7.66 (1H, d, ArH), 7.64 (1H, s, ArH), 7.27 (1H, d, ArH), 6.29 (1H, m, NH), 5.95 (1H, d, ArH), 3.58 (3H, s, CH3), 3.45 (3H, s, CH3), 3.04 (3H, d, CH3), 2.17 (3H, s, CH3).

### Compound 31: 3-Methyl-4-(methyl(1-methyl-1H-imidazo[4,5-c]pyridin-6-yl)amino)benzonitrile and

### Compound 32: 3-Methyl-4-(methyl(1-methyl-1H-imidazo[4,5-c]pyridin-6-yl)amino)benzamide

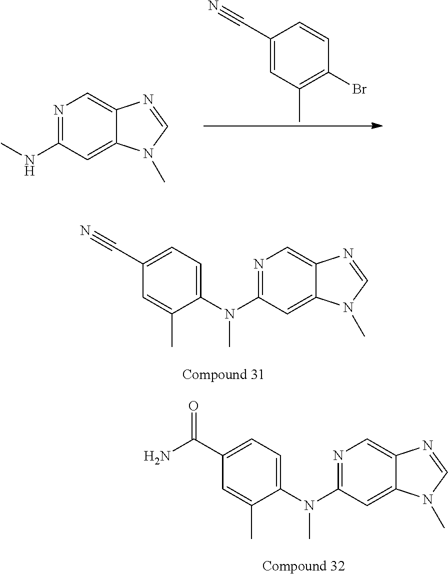

N,1-Dimethyl-1H-imidazo[4,5-c]pyridin-6-amine (150 mg, 0.92 mmol), 4-bromo-3-methylbenzonitrile (198 mg, 1.01 mmol), 2-dicyclohexylphosphino-2′,4′,6′-triisopropylbiphenyl (44 mg, 0.092 mmol) and sodium tert-butoxide (133 mg, 1.38 mmol) were stirred in toluene (4 mL) and N₂(g) was bubbled through the mixture for 10 min. Tris(dibenzylideneacetone)dipalladium(0) (42 mg, 0.046 mmol) was added and the resulting mixture heated in a sealed tube at 100° C. for 1 h. The reaction mixture was cooled to room temperature and DCM and water were added. The aqueous phase was extracted with DCM and the combined organics filtered through a hydrophobic frit and then concentrated in vacuo. The residue was dissolved in DCM (5 mL) and passed through a PL-Thiol MP SPE+ column. DCM was passed through the column and the filtrate concentrated in vacuo. The residue was purified by preparative HPLC to give the desired compounds 3-Methyl-4-(methyl(1-methyl-1H-imidazo[4,5-c]pyridin-6-yl)amino)benzonitrile (compound 31) and 3-Methyl-4-(methyl(1-methyl-1H-imidazo[4,5-c]pyridin-6-yl)amino)benzamide (compound 32)

### Compounds 33/38/45: N-(4-(4-Methoxybenzyloxy)-2-methylphenyl)-N,1-dimethyl-1H-imidazo[4,5-d]pyridin-6-amine (Compound 33) and 3-Methyl-4-(methyl(1-methyl-1H-imidazo[4,5-c]pyridin-6-yl)amino)phenol (Compound 38) and N-(4-Methoxy-2-methylphenyl)-N,1-dimethyl-1H-imidazo[4,5-d]pyridin-6-amine (Compound 45)

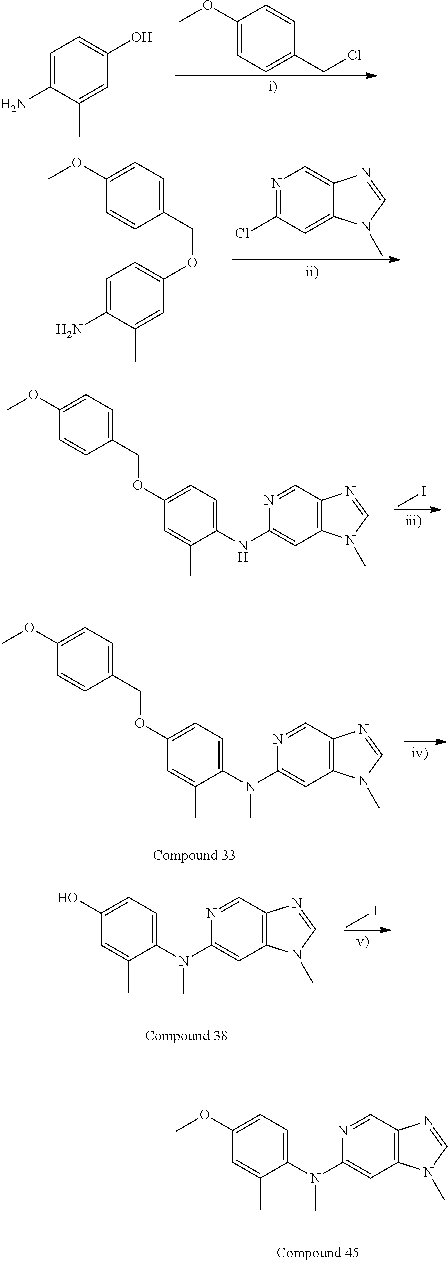

### Step i): 4-(4-Methoxybenzyloxy)-2-methylaniline

1-(Chloromethyl)-4-methoxybenzene (17 g, 0.1 mol) was added to a stirred suspension of cesium carbonate (48.9 g, 0.15 mol) and 4-amino-3-methylphenol (12.2 g, 0.1 mol) in DMF (200 mL) and the resulting suspension was stirred at room temperature for 1 d. The mixture was concentrated in vacuo and the residue was treated with water and extracted with DCM. The combined extracts were dried (MgSO₄), filtered and concentrated in vacuo to give a brown oil which was purified by column chromatography using silica gel (40% to 75% EtOAc in isohexanes) to give the desired compound.

¹H NMR δ (ppm)(DMSO-d₆): 7.32 (2H, d, ArH), 6.97-6.89 (2H, m, ArH), 6.64 (1H, d, ArH), 6.61-6.50 (2H, m, ArH), 4.85 (2H, s, CH2), 4.36 (2H, s, NH₂), 3.75 (3H, s, CH3), 2.03 (3H, s, CH3).

### Step ii) N-(4-(4-Methoxybenzyloxy)-2-methylphenyl)-1-methyl-1H-imidazo[4,5-c]pyridin-6-amine

6-Chloro-1-methyl-1H-imidazo[4,5-c]pyridine (2.52 g, 0.015 mol) was stirred with 4,5-bis(diphenylphosphino)-9,9-dimethylxanthene (0.86 g, 0.0015 mol), sodium tert-butoxide (2.16 g, 0.0225 mol) and 4-(4-methoxybenzyloxy)-2-methylaniline (3.68 g, 0.015 mol) in dry 1,4-dioxane (50 mL) under N₂ (g) for 30 min. Tris(dibenzylideneacetone)dipalladium(0) (0.68 g, 0.00075 mol) was added and the mixture was stirred at 100° C. for 4.5 h. The mixture was concentrated in vacuo and the residue was treated with water (50 mL) and extracted into EtOAc (2×300 mL). The combined extracts were washed with brine, dried (MgSO₄), filtered and concentrated in vacuo to give a brown solid which was recrystalised from isopropanol to give the desired compound.

¹H NMR δ (ppm)(DMSO-d₆): 8.43 (1H, d, ArH), 7.98 (1H, s, NH), 7.75 (1H, s, ArH), 7.39 (2H, d, ArH), 7.27 (1H, d, ArH), 7.00-6.90 (3H, m, ArH), 6.82 (1H, dd, ArH), 6.38 (1H, d, ArH), 5.00 (2H, s, CH), 3.77 (3H, s, CH3), 3.64 (3H, s, CH3), 2.17 (3H, s, CH3).

LCMS (15 cm_Bicarb_GeminiNX_HPLC_MeCN) Rt 9.63 (min) m/z 375 (MH⁺).

### Step iii): N-(4-(4-Methoxybenzyloxy)-2-methylphenyl)-N,1-dimethyl-1H-imidazo[4,5-c]pyridin-6-amine

Sodium hydride (0.38 g of 60% dispersion in mineral oil, 9.4 mmol) was added to a stirred solution of N-(4-(4-methoxybenzyloxy)-2-methylphenyl)-1-methyl-1H-imidazo[4,5-c]pyridin-6-amine (3.25 g, 8.6 mmol) in dry DMF (80 mL) under nitrogen. The reaction mixture was stirred at room temperature for 1 h before the addition of iodomethane (1.83 g, 12.9 mmol) and stirring was then continued for 5 h. The reaction mixture was concentrated in vacuo and the residue was treated with water (50 mL) and extracted with EtOAc, the combined extracts were dried (MgSO₄), filtered and concentrated in vacuo to give the desired compound (3.3 g) as a brown oil which was used without further purification. A sample of this material (100 mg) was purified by preparative HPLC to give the desired compound (Compound 33)

¹H NMR δ (ppm)(DMSO-d₆): 8.49 (1H, d, ArH), 7.96 (1H, s, ArH), 7.41 (2H, d, ArH), 7.11 (1H, d, ArH), 7.02-6.90 (4H, m, ArH), 6.04 (1H, s, ArH), 5.03 (2H, s, CH2), 3.77 (3H, s, CH3), 3.57 (3H, s, CH3), 3.32 (3H, d, CH3), 2.12-1.99 (3H, m, CH3).

LCMS (15 cm_Bicarb_GeminiNX_HPLC_MeCN) Rt 10.11 (min) m/z 389 (MH⁺).

### Step iv): 3-Methyl-4-(methyl(1-methyl-1H-imidazo[4,5-c]pyridin-6-yl)amino)phenol

N-(4-(4-Methoxybenzyloxy)-2-methylphenyl)-N,1-dimethyl-1H-imidazo[4,5-c]pyridin-6-amine (3.2 g) was dissolved in DCM (15 mL) and trifluoroacetic acid (15 mL) was added. The resulting solution was allowed to stand for 4 h. The reaction was concentrated in vacuo and the residue was loaded onto a 70 g SCX cartridge and washed with MeOH until the washings were neutral. The product was eluted with MeOH/ammonia to give the desired compound (Compound 38). A sample of this material (100 mg) was purified by preparative HPLC to give the desired compound (28 mg).

¹H NMR δ (ppm)(DMSO-d₆): 9.38 (1H, s, OH), 8.49 (1H, d, ArH), 7.94 (1H, s, ArH), 6.98 (1H, d, ArH), 6.74 (1H, d, ArH), 6.68 (1H, dd, ArH), 5.97 (1H, d, ArH), 3.55 (3H, s, CH3), 3.37-3.24 (3H, m, CH3), 1.99 (3H, s, CH3).

LCMS (10 cm_Formic_ACE-AR_HPLC_MeOH_Slow) Rt 8.92 (min) m/z 269 (MH⁺).

### Step v): N-(4-Methoxy-2-methylphenyl)-N,1-dimethyl-1H-imidazo[4,5-c]pyridin-6-amine

Iodomethane (0.11 g, 0.8 mmol) was added to a stirred suspension of 3-methyl-4-(methyl(1-methyl-1H-imidazo[4,5-c]pyridin-6-yl)amino)phenol (0.107 g, 0.4 mmol) and cesium carbonate (0.195 g, 0.6 mmol) in DMF (1 mL). The mixture was stirred at 50° C. for 6 h, filtered and purified by preparative HPLC to give the desired compound (Compound 45).

¹H NMR δ (ppm)(DMSO-d₆): 8.49 (1H, d, ArH), 8.02-7.86 (1H, m, ArH), 7.10 (1H, m, ArH), 6.93 (1H, d, ArH), 6.86 (1H, dd, ArH), 6.03 (1H, d, ArH), 3.88-3.68 (3H, m, CH3), 3.57 (3H, s, CH3), 3.35-3.28 (3H, m, CH3), 2.15-1.98 (3H, m, CH3).

LCMS (15 cm_Formic_ASCENTIS_HPLC_MeCN) Rt 7.00 (min) m/z 283 (MH⁺).

### Compound 34: N-Ethyl-N-(2-ethylphenyl)-1-methyl-1H-imidazo[4,5-c]pyridin-6-amine

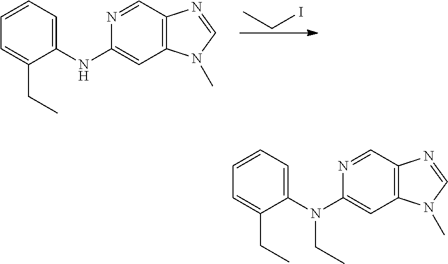

A solution of N-(2-ethylphenyl)-1-methyl-1H-imidazo[4,5-c]pyridin-6-amine (200 mg, 0.79 mmol) in DMF (3 mL) was added drop wise to a suspension of sodium hydride (60% dispersion in mineral oil, 48 mg, 1.19 mmol) in DMF (1 mL) and stirred at room temperature for 1 h. Iodoethane (76 μL, 0.95 mmol) was added and the reaction mixture stirred for 1 h at room temperature. The reaction mixture was partitioned between DCM and water, the layers were separated and the aqueous layer extracted with DCM. The organics were combined, filtered through a hydrophobic filter then concentrated in vacuo. The resulting residue was purified by preparative HPLC to give the desired compound.

¹H NMR δ (ppm)(DMSO-d₆): 8.50 (1H, d, ArH), 7.95 (1H, s, ArH), 7.46-7.42 (1H, m, ArH), 7.36-7.29 (2H, m, ArH), 7.21-7.16 (1H, m, ArH), 5.91 (1H, d, ArH), 3.89 (2H, d, CH), 3.53 (3H, s, CH3), 2.50-2.40 (2H, m, CH), 1.21-1.05 (6H, m, CH3).

LCMS (15 cm_Formic_ASCENTIS_HPLC_MeCN) Rt 7.26 (min) m/z 281 (MH⁺).

### Compound 37: N-Cyclopropyl-N-(2-ethylphenyl)-1-methyl-1H-imidazo[4,5-c]pyridin-6-amine

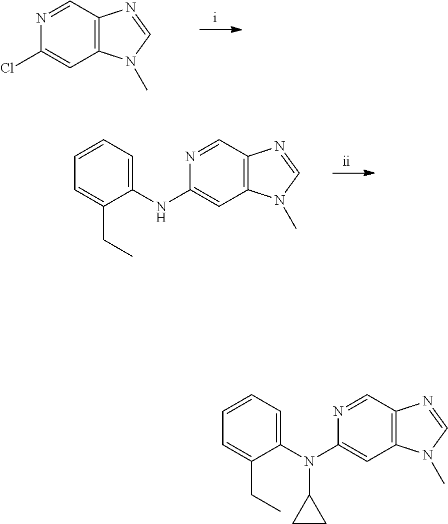 [SMILES: Cn1cnc2cnc(Cl)cc21 ; CCc1ccccc1Nc1cc2c(cn1)ncn2C ; CCc1ccccc1N(c1cc2c(cn1)ncn2C)C1CC1]

### Step i): N-(2-Ethylphenyl)-1-methyl-1H-imidazo[4,5-c]pyridin-6-amine

To stirred degassed 1,4-dioxane (40 mL) was added 6-chloro-1-methyl-1H-imidazo[4,5-c]pyridine (3 g, 0.018 mol), 2-ethylaniline (2.44 mL, 0.0198 mol), sodium tert-butoxide (2.6 g, 0.027 mol), tris(dibenzylideneacetone)dipalladium(0) (0.824 g, 0.9 mmol) and 4,5-bis(diphenylphosphino)-9,9-dimethylxanthene (1.04 g, 0.0018 mol). The reaction was heated to 90° C. for 1 d and cooled to room temperature. The reaction mixture was filtered through Celite, washed through with EtOAc and partitioned between EtOAc and water. The layers were separated and the aqueous layer further extracted with EtOAc, the organics were combined, dried (MgSO₄), filtered and concentrated in vacuo to give the desired compound, which was used in the next step without further purification.

¹H NMR δ (ppm)(DMSO-d₆): 8.48 (1H, s, NH), 8.02 (1H, s, ArH), 7.94-7.78 (1H, m, ArH), 7.68-7.44 (1H, m, ArH), 7.24-7.11 (2H, m, ArH), 7.08-7.00 (1H, m, ArH), 6.66 (1H, d, ArH), 3.68 (3H, s, CH3), 2.70-2.60 (2H, m, CH2), 1.16-1.07 (3H, m, CH3).

### Step ii): N-Cyclopropyl-N-(2-ethylphenyl)-1-methyl-1H-imidazo[4,5-c]pyridin-6-amine

A warm mixture of 2,2′-bipyridine (124 mg, 0.79 mmol) and copper(II) acetate (144 mg 0.79 mmol) in CHCl₃ (3 mL) was added quickly to a vigorously stirred solution of N-(2-ethylphenyl)-1-methyl-1H-imidazo[4,5-c]pyridin-6-amine (200 mg, 0.79 mmol), cyclopropylboronic acid (136 mg, 1.58 mmol) and sodium carbonate (168 mg, 1.58 mmol) at room temperature. The reaction mixture was stirred in air for 1 d. NH₄Cl solution (sat. aq. 8 mL) was added and the mixture stirred for 10 min at room temperature. The reaction mixture was diluted with DCM and washed with water. The layers were separated, the aqueous layer extracted with DCM, organics were combined and dried (MgSO₄) concentrated in vacuo and the residue was purified by preparative HPLC to give the desired compound.

¹H NMR δ (ppm)(DMSO-d₆): 8.38 (1H, d, ArH), 8.03 (1H, s, ArH), 7.33-7.29 (1H, m, ArH), 7.25-7.20 (2H, m, ArH), 7.09-7.05 (1H, m, ArH), 6.86 (1H, d, ArH), 3.74 (3H, s, CH3), 3.03-2.97 (1H, m, CH), 2.41-2.33 (2H, m, CH), 1.07-1.00 (3H, m, CH), 0.92-0.86 (2H, m, CH), 0.51-0.45 (2H, m, CH).

LCMS (10 cm_ESCI_Bicarb_MeCN) Rt 3.16 (min) m/z 293 (MH⁺).

### Compound 40: 3-methyl-4-(methyl(1-methyl-1H-imidazo[4,5-c]pyridin-6-yl)amino)benzoic acid, and

### Compound 50: N-(2-methoxyethyl)-3-methyl-4-(methyl(1-methyl-1H-imidazo[4,5-c]pyridin-6-yl)amino)benzamide

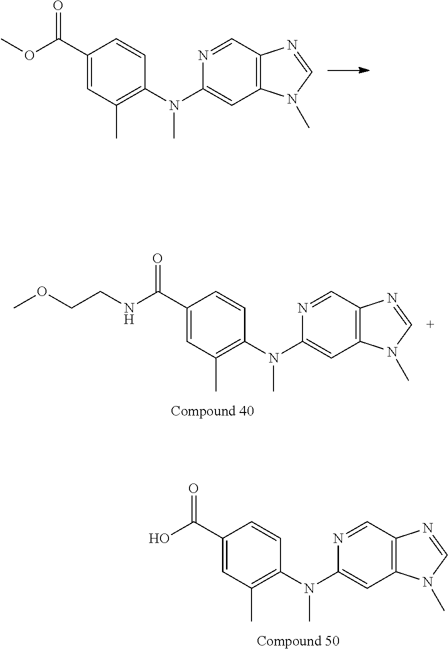 [SMILES: COC(=O)c1ccc(N(C)c2cc3c(cn2)ncn3C)c(C)c1 ; COCCNC(=O)c1ccc(N(C)c2cc3c(cn2)ncn3C)c(C)c1 ; Cc1cc(C(=O)O)ccc1N(C)c1cc2c(cn1)ncn2C]

Trimethyl aluminium (0.11 mL, 0.225 mmol) was added to a solution of 2-methoxyethanamine (20 μL, 0.225 mmol) in CHCl₃ (1 mL) under a N₂ (g) atmosphere. The reaction was stirred at room temperature for 20 min then methyl 3-methyl-4-(methyl(1-methyl-1H-imidazo[4,5-c]pyridin-6-yl)amino)benzoate (70 mg, 0.225 mmol) in CHCl₃ (1 mL) was added and the reaction was heated to 50° C. for 1 d. The reaction mixture was diluted with EtOAc and washed with 1 M Na₂CO₃ aq. solution then brine. The organic phase was dried (MgSO₄), filtered and concentrated in vacuo. The crude material was purified by preparative HPLC to give the desired compounds: N-(2-Methoxyethyl)-3-methyl-4-(methyl(1-methyl-1H-imidazo[4,5-c]pyridin-6-yl)amino)benzamide (Compound 50), and 3-Methyl-4-(methyl(1-methyl-1H-imidazo[4,5-c]pyridin-6-yl)amino)benzoic acid (Compound 40).

### Compound 41:2-(Methyl(1-methyl-1H-imidazo[4,5-c]pyridin-6-yl)amino)benzonitrile

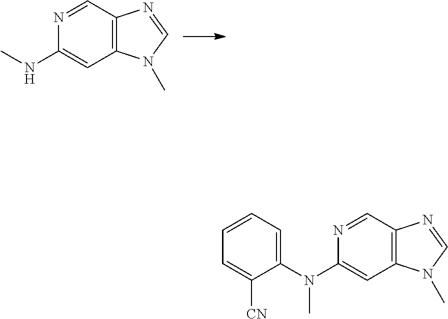 [SMILES: CNc1cc2c(cn1)ncn2C ; CN(c1cc2c(cn1)ncn2C)c1ccccc1C#N]

N,1-Dimethyl-1H-imidazo[4,5-c]pyridin-6-amine (200 mg, 1.23 mmol), 2-bromobenzonitrile (248 mg, 1.36 mmol), 2-dicyclohexylphosphino-2′,4′,6′-triisopropylbiphenyl (59 mg, 0.12 mmol) and sodium tert-butoxide (177 mg, 1.85 mmol) were stirred in toluene (5 mL) and N₂ (g) was bubbled through the mixture for 10 min Tris(dibenzylideneacetone)dipalladium(0) (56 mg, 0.062 mmol) was added and the resulting mixture heated in a sealed tube at 100° C. for 2 h. The reaction mixture was cooled to room temperature and DCM and water were added. The aqueous phase was extracted with DCM and the combined organics were filtered through a hydrophobic frit and concentrated in vacuo. The residue was dissolved in DCM (5 mL) and passed through a PL-Thiol MP SPE+ column. DCM was passed through the column and the filtrate concentrated in vacuo. The residue was purified by preparative HPLC to give the desired compound.

¹H NMR δ (ppm)(DMSO-d₆): 8.49 (1H, d, ArH), 8.11 (1H, s, ArH), 7.82 (1H, dd, ArH), 7.73 (1H, ddd, ArH), 7.45 (1H, dd, ArH), 7.38-7.33 (1H, m, ArH), 6.87 (1H, d, ArH), 3.75 (3H, s, CH3), 3.47 (3H, s, CH3).

LCMS (10 cm_ESCI_Bicarb_MeCN) Rt 2.88 (min) m/z 264 (MH⁺).

Compound 68: N-(2-Chlorophenyl)-N,1-dimethyl-1H-imidazo[4,5-c]pyridin-6-amine

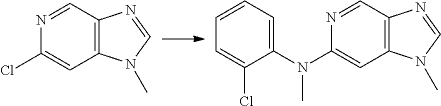 [SMILES: Cn1cnc2cnc(Cl)cc21 ; CN(c1cc2c(cn1)ncn2C)c1ccccc1Cl]

6-Chloro-1-methyl-1H-imidazo[4,5-c]pyridine (200 mg, 1.19 mmol), 2-chloro-N-methylaniline (185 mg, 1.31 mmol), 2-dicyclohexylphosphino-2′,4′,6′-triisopropylbiphenyl (57 mg, 0.12 mmol) and sodium tert-butoxide (172 mg, 1.79 mmol) were stirred in 1,4-dioxane (5 mL) and N₂(g) was bubbled through the mixture for 10 min. Tris(dibenzylideneacetone)dipalladium(0) (54 mg, 0.06 mmol) was added and the dark mixture heated in a sealed tube at 100° C. for 2 h. The reaction mixture was cooled to room temperature and DCM and water were added. The aqueous phase was extracted with DCM and the combined organics filtered through a hydrophobic frit and then concentrated in vacuo. The residue was dissolved in DCM (5 mL) and passed through a PL-Thiol MP SPE+ column. DCM was passed through the column and the filtrate concentrated in vacuo. The residue was purified by preparative HPLC to give the desired compound.

¹H NMR δ (ppm)(DMSO-d₆): 8.47 (1H, s, ArH), 8.02 (1H, s, ArH), 7.59 (1H, d, ArH), 7.47-7.38 (2H, m, ArH), 7.39-7.31 (1H, m, ArH), 6.39 (1H, s, ArH), 3.66 (3H, s, CH₃), 3.35 (3H, s, CH₃).

LCMS (10 cm_ESCI_Bicarb_MeCN) Rt 3.22 (min) m/z 273 (MH⁺).

### Compound 71: N,1-Dimethyl-N-(2-methyl-4-(1H-pyrazol-4-yl)phenyl)-1H-imidazo[4,5-c]pyridin-6-amine

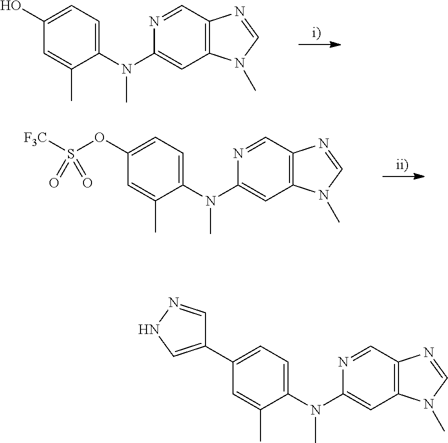

### Step i): 3-Methyl-4-(methyl(1-methyl-1H-imidazo[4,5-c]pyridin-6-yl)amino)phenyl trifluoromethanesulfonate

Triethyl amine (0.125 mL, 0.89 mmol) was added to a solution of 3-methyl-4-(methyl(1-methyl-1H-imidazo[4,5-c]pyridin-6-yl)amino)phenol (200 mg, 0.746 mmol) and N-phenyl bis(trifluoromethanesulphonimide) (293 mg, 0.82 mmol) in DMF (4 mL). The reaction was stirred at room temperature for 16 h then diluted with EtOAc then washed with water then brine. The organic layer was dried (MgSO₄), filtered and concentrated in vacuo to give the desired compound which was used in the next step without further purification.

### Step ii): N,1-Dimethyl-N-(2-methyl-4-(1H-pyrazol-4-yl)phenyl)-1H-imidazo[4,5-c]pyridin-6-amine

[1,1′-Bis(diphenylphosphino)ferrocene]dichloropalladium(II) (20 mg, 0.022 mmol) was added to a solution of 3-methyl-4-(methyl(1-methyl-1H-imidazo[4,5-c]pyridin-6-yl)amino)phenyl trifluoromethanesulfonate (90 mg, 0.22 mmol), (1H-pyrazol-4-yl)boronic acid (37 mg, 0.33 mmol) and cesium carbonate (3.7 M aq. soln, 120 μL, 0.45 mmol) in DMF (3 mL). The reaction was heated to 80° C. for 4 h then diluted with EtOAc then washed with water then brine. The organic layer was dried (MgSO₄), filtered and concentrated in vacuo. The crude material was purified by preparative HPLC to give the desired compound.

¹H NMR δ (ppm)(CHCl₃-d): 8.73 (1H, d, ArH), 7.89 (2H, s, ArH), 7.66 (1H, s, NH), 7.49-7.42 (2H, m, ArH), 7.23 (2H, d, ArH), 5.99 (1H, d, ArH), 3.59 (3H, s, CH₃), 3.47 (3H, s, CH₃), 2.19 (3H, s, CH₃).

LCMS (10 cm_ESCI_Formic_MeCN) Rt 2.17 (min) m/z 319 (MH⁺).

### Compound 74: N-(4-(4-methoxybenzyl)-2-methylphenyl)-N,1-dimethyl-1H-imidazo[4,5-c]pyridin-6-amine

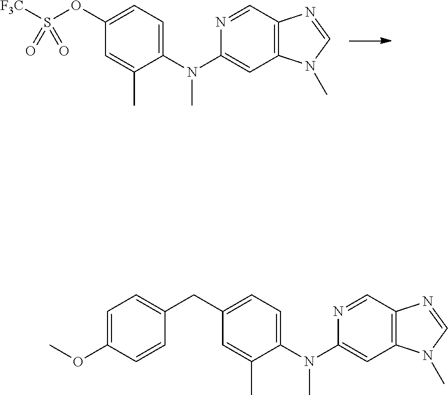 [SMILES: Cc1cc(OS(=O)(=O)C(F)(F)F)ccc1N(C)c1cc2c(cn1)ncn2C ; COc1ccc(Cc2ccc(N(C)c3cc4c(cn3)ncn4C)c(C)c2)cc1]

(4-Methoxybenzyl)zinc bromide (0.8 mL, 0.4 mmol, 0.5 M in THF) was added to a solution of [1,1′-bis(diphenylphosphino)ferrocene]dichloropalladium(II) (9 mg, 0.01 mmol) and 3-methyl-4-(methyl(1-methyl-1H-imidazo[4,5-c]pyridin-6-yl)amino)phenyl trifluoromethanesulfonate (40 mg, 0.1 mmol) in THF (1 mL). The reaction was heated to 70° C. for 4 h then diluted with EtOAc and washed with water then brine. The organic layer was dried (MgSO₄), filtered and concentrated in vacuo. The crude material was purified by preparative HPLC to give the desired compound.

¹H NMR δ (ppm)(CHCl₃-d): 8.71 (1H, s, ArH), 7.60 (1H, s, ArH), 7.20-7.05 (5H, m, ArH), 6.87 (2H, d, ArH), 5.89 (1H, s, ArH), 3.94 (2H, s, CH₂), 3.80 (3H, s, CH₃), 3.56 (3H, s, CH₃), 3.42 (3H, s, CH₃), 2.10 (3H, s, CH₃).

LCMS (10 cm_Formic_ACE-AR_HPLC_MeOH_Slow) Rt 12.13 (min) m/z 373 (MH⁺).

### Compound 80: (3-Ethyl-4-(methyl(1-methyl-1H-imidazo[4,5-c]pyridin-6-yl)amino)phenyl)-MeOH; and

### Compound 81: (3-Ethyl-4-(methyl(1-methyl-1H-imidazo[4,5-c]pyridin-6-yl)amino)phenyl)MeOH

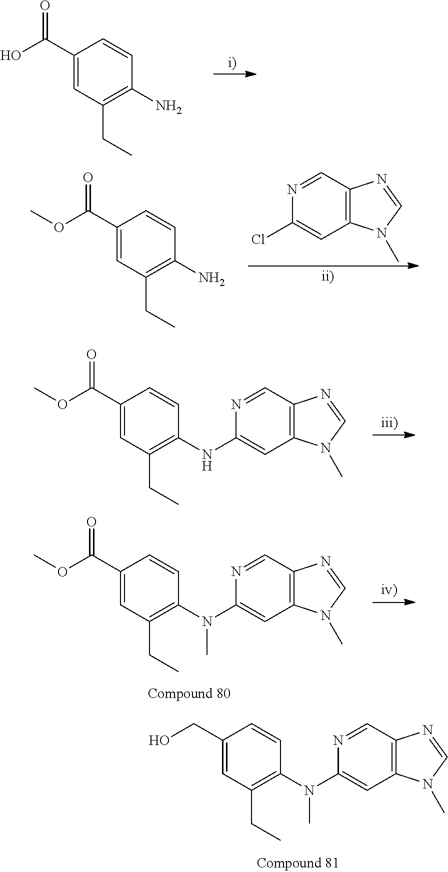

### Step i): Methyl 4-amino-3-ethylbenzoate

Hydrochloric acid (4 mL, 4 M solution in 1,4-dioxane) was added to a solution of 4-amino-3-ethylbenzoic acid (1.5 g) in MeOH (20 mL) in a Kymax tube. The tube was sealed and heated to 65° C. for 16 h. The reaction was concentrated in vacuo, diluted with EtOAc and washed with 1 M Na₂CO₃ aq. solution then brine. The organic layer was dried (MgSO₄), filtered and concentrated in vacuo to give the desired compound which was used in the next step without further purification.

### Step ii): Methyl 3-ethyl-4-(1-methyl-1H-imidazo[4,5-c]pyridin-6-ylamino)benzoate

Methyl 4-amino-3-ethylbenzoate (0.9 g, 5.02 mmol), 6-chloro-1-methyl-1H-imidazo[4,5-c]pyridine (0.802 g, 4.788 mmol) and sodium tert-butoxide (0.689 g, 7.18 mmol) were suspended in 1,4-dioxane (40 mL). The reaction was stirred under an atmosphere of N₂ (g) for 20 min then tris(dibenzylideneacetone)dipalladium(0) (219 mg, 0.23 mmol) and 2-dicyclohexylphosphino-2′,4′,6′-triisopropylbiphenyl (228 mg, 0.478 mmol) were added. The reaction was heated to 85° C. for 4 h then concentrated in vacuo. The crude material was dissolved in MeOH (40 mL) and HCl (4 mL, 4 M in dioxane) and the reaction was refluxed for 4 h to re-esterify the hydrolysed ester. The reaction was concentrated in vacuo, diluted with EtOAc and washed with water then brine. The organic layer was dried (MgSO₄), filtered and concentrated in vacuo. The residue was purified by column chromatography using silica gel and eluting with DCM:MeOH 97:3 to give the desired compound.

### Step iii): Methyl 3-ethyl-4-(methyl(1-methyl-1H-imidazo[4,5-c]pyridin-6-yl)amino)benzoate (Compound 80)

Sodium hydride (160 mg, 3.97 mmol, 60% dispersion in mineral oil) was added to a solution of methyl 3-ethyl-4-(1-methyl-1H-imidazo[4,5-c]pyridin-6-ylamino)benzoate (823 mg, 2.65 mmol) in DMF (10 mL) at 0° C. The reaction was stirred under an N₂ (g) atmosphere until effervescence stopped. Iodomethane (0.24 mL, 3.97 mmol) was added and the reaction was allowed to warm gradually to room temperature. The reaction was diluted with EtOAc and washed with water then brine. The organic layer was dried (MgSO₄), filtered and concentrated in vacuo. The residue was purified by column chromatography using silica gel and eluting with DCM:MeOH 98:2 to give the desired compound.

¹H NMR δ (ppm)(CHCl₃-d): 8.73 (1H, s, ArH), 8.08 (1H, s, ArH), 7.97 (1H, dd, ArH), 7.64 (1H, s, ArH), 7.27 (1H, d, ArH), 5.93 (1H, d, ArH), 3.95 (3H, s, CH₃), 3.57 (3H, s, CH₃), 3.47 (3H, s, CH₃), 2.56 (2H, q, CH₂), 1.19 (3H, t, CH₃).

LCMS (15 cm_Bicarb_GeminiNX_HPLC_MeCN) Rt 9.47 (min) m/z 325 (MH⁺).

### Step iv): (3-Ethyl-4-(methyl(1-methyl-1H-imidazo[4,5-c]pyridin-6-yl)amino)phenyl)MeOH (Compound 81)

Methyl 3-ethyl-4-(methyl(1-methyl-1H-imidazo[4,5-c]pyridin-6-yl)amino)benzoate (300 mg, 0.92 mmol) was dissolved in THF (3 mL) and cooled to 0° C. under N₂ (g). Lithium aluminum hydride (0.92 mL, 0.92 mmol, 1 M in THF) was added slowly and the reaction mixture was allowed to warm to room temperature over 1 h. Concentrated NaOH solution (aq. 3 mL) was added slowly followed by water (3 mL). The reaction mixture was extracted with EtOAc the organic phase was washed with water and brine then dried (MgSO₄), filtered and concentrated in vacuo. The crude material was purified by column chromatography using silica gel and eluting with DCM:MeOH 97:3 to give the desired compound.

¹H NMR δ (ppm)(CHCl₃-d): 8.70 (1H, d, ArH), 7.61 (1H, s, ArH), 7.41 (1H, d, ArH), 7.30 (1H, m, ArH), 7.20 (1H, d, ArH), 5.90 (1H, d, ArH), 4.76 (2H, s, CH2), 3.56 (3H, s, CH3), 3.44 (3H, s, CH3), 2.54 (2H, q, CH2), 2.25 (1H, s, OH), 1.16 (3H, t, CH3).

LCMS (10 cm_ESCI_Formic_MeCN) Rt 2.15 (min) m/z 297 (MH⁺).

### Compound 85: 2-(7-(Methyl(1-methyl-1H-imidazo[4,5-c]pyridin-6-yl)amino)-3-oxo-2H-benzo[b][1,4]oxazin-4(3H)-yl)acetonitrile

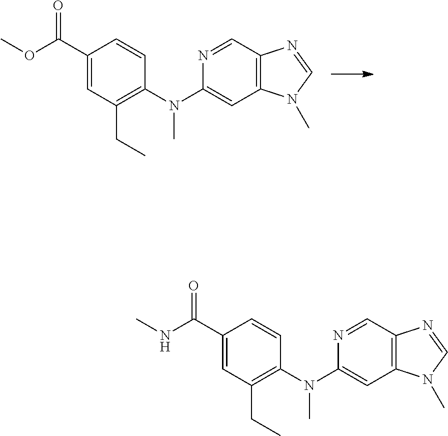 [SMILES: CCc1cc(C(=O)OC)ccc1N(C)c1cc2c(cn1)ncn2C ; CCc1cc(C(=O)NC)ccc1N(C)c1cc2c(cn1)ncn2C]

Methyl amine (33 wt % in ethanol) (3 mL) was added to methyl 3-ethyl-4-(methyl(1-methyl-1H-imidazo[4,5-c]pyridin-6-yl)amino)benzoate (40 mg, 0.12 mmol) in a microwave tube. The reaction was sealed and heated in the microwave to 150° C. for 1 h. The reaction was concentrated in vacuo and the residue was purified by preparative HPLC to give the desired compound.

¹H NMR δ (ppm)(DMSO-d₆): 8.48 (1H, s, ArH), 8.44 (1H, d, ArH), 8.00 (1H, s, ArH), 7.85 (1H, d, ArH), 7.75 (1H, dd, ArH), 7.26 (1H, d, ArH), 6.24 (1H, m, NH), 3.62 (3H, s, CH₃), 3.35 (3H, s, CH₃), 2.81 (3H, d, CH₃), 2.50 (2H, m, CH₂), 1.12 (3H, t, CH₃).

LCMS (10 cm_ESCI_Bicarb_MeCN) Rt 2.30 (min) m/z 324 (MH⁺).

### Compound 86: N-(2-Cyanoethyl)-3-ethyl-4-(methyl(1-methyl-1H-imidazo[4,5-c]pyridin-6-yl)amino)benzamide

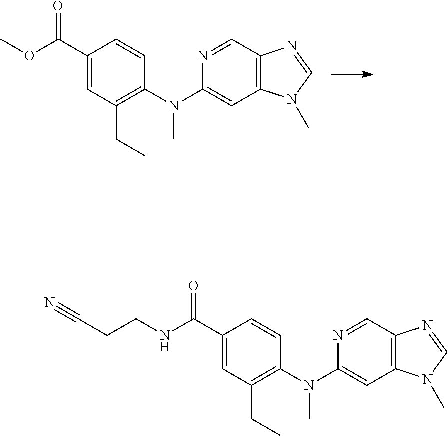 [SMILES: CCc1cc(C(=O)OC)ccc1N(C)c1cc2c(cn1)ncn2C ; CCc1cc(C(=O)NCCC#N)ccc1N(C)c1cc2c(cn1)ncn2C]

3-Aminopropionitrile (17 μL, 0.23 mmol) was added to a solution of bis(trimethylaluminum)-1,4-diazobicyclo[2.2.2]octane adduct (60 mg, 0.231 mmol) in THF (1 mL). The reaction was heated to 70° C. for 30 min and then methyl 3-ethyl-4-(methyl(1-methyl-1H-imidazo[4,5-c]pyridin-6-yl)amino)benzoate (50 mg, 0.154 mmol) in THF (1 mL) was added and the reaction mixture was heated at 70° C. under an N₂ (g) atmosphere for 1 d. The reaction was diluted with EtOAc then the organic phase was washed with NaHCO₃ (1 M aq. solution) and brine then dried (MgSO₄), filtered and concentrated in vacuo. The residue was purified by preparative HPLC to give the desired compound.

¹H NMR δ (ppm)(CHCl₃-d): 8.71 (1H, d, ArH), 7.85 (1H, d, ArH), 7.76-7.64 (3H, m, ArH), 6.97 (1H, t, NH), 5.94 (1H, d, ArH), 3.75 (2H, m, CH₂), 3.59 (3H, s, CH₃), 3.45 (3H, s, CH₃), 2.80 (2H, t, CH₂), 2.55 (2H, q, CH₂), 1.17 (3H, t, CH₃).

### Compound 87: 4-Ethyl-N,N-dimethyl-3-(methyl(1-methyl-1H-imidazo[4,5-c]pyridin-6-yl)amino)benzamide

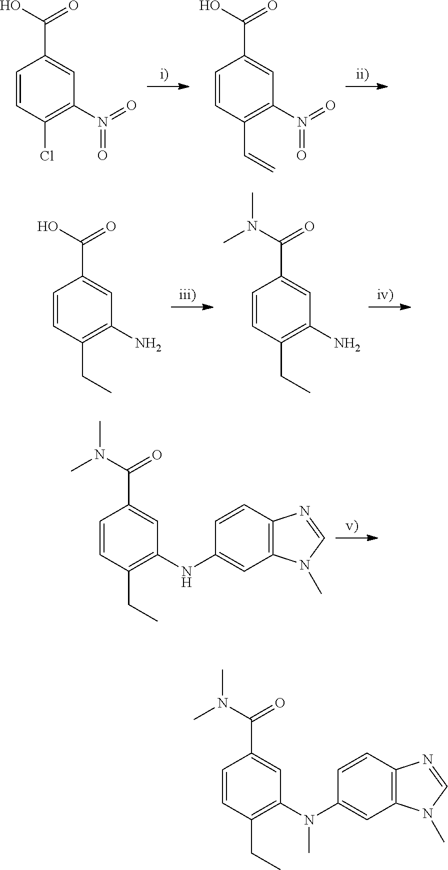

### Step i) 3-Nitro-4-vinylbenzoic acid

A mixture of 4-chloro-3-nitrobenzoic acid (5 g, 0.0248 mol), potassium vinyl trifluoroborate (3.67 g, 0.0273 mol) and cesium carbonate (20 g, 0.062 mol) was stirred in DMF (100 mL) and water (20 mL) under N₂ for 20 min. At this point, 1,1′-bis(diphenylphosphino)ferrocene]dichloropalladium(II) (2.2 g, 2.69 mmol) was added and the mixture was stirred at 100° C. for 18 h. The solvent was removed in vacuo and the residue was dissolved in water and filtered through Celite. The filtrate was acidified (pH 1) with 2 M HCl (aqueous) and the resulting solid was filtered, washed with water and dried in a vacuum oven to give the desired compound.

¹H NMR δ (ppm)(DMSO-d₆): 13.62 (1H, s, OH), 8.40 (1H, d, ArH), 8.20 (1H, dd, ArH), 7.95 (1H, d, ArH), 7.07 (1H, m, CH), 6.04 (1H, d, CH), 5.69-5.60 (1H, m, CH).

### Step ii) 3-Amino-4-ethylbenzoic acid

Palladium on carbon (10% wt., 1.5 g) was added to a solution of 3-nitro-4-vinylbenzoic acid

(1.2 g, 0.0062 mol) and ammonium formate (3.78 g, 0.06 mol) in MeOH (20 mL) under N₂. The mixture was gently refluxed for 2 h and the cooled reaction mixture was filtered through Celite. The filtrate was concentrated in vacuo and the residue was treated with water (4 mL), filtered and dried to give the desired compound.

¹H NMR δ (ppm)(DMSO-d₆): 12.40 (1H, s, OH), 7.23 (1H, d, ArH), 7.09 (1H, dd, ArH), 7.01 (1H, d, ArH), 5.07 (2H, s, NH₂), 2.53-2.44 (2H, q, CH₂), 1.13 (3H, t, CH₃).

### Step iii) 3-Amino-4-ethyl-N,N-dimethylbenzamide

Dimethylamine (1.5 mL of a 2 M solution in MeOH, 0.003 mol) was added to a stirred solution of 3-amino-4-ethylbenzoic acid (0.247 g, 0.0015 mol), Et₃N (0.45 g, 0.0045 mol) and (2-(7-aza-1H-benzoazole-1-yl)-1,1,3,3-tetramethyluronium hexafluorophosphate) (0.627 g, 0.00165 mol) in DMF (5 mL). After stirring for 6 h at room temperature the reaction mixture was concentrated in vacuo and the residue was dissolved in EtOAc and washed with saturated aqueous sodium carbonate solution. The organics were dried (MgSO₄) and concentrated in vacuo. The residue was dissolved in MeOH and loaded onto a 10 g SCX column. Methanol was washed through and the compound was eluted with 7 N NH₃ in MeOH:MeOH (1:4). The eluent was concentrated in vacuo to give the desired compound which was used in the next step without further purification.

¹H NMR δ (ppm)(DMSO-d₆): 6.95 (1H, d, ArH), 6.61 (1H, d, ArH), 6.49 (1H, dd, ArH), 4.99 (2H, s, NH2), 3.07-2.74 (6H, m, CH₃), 2.49-2.40 (2H, m, CH), 1.13 (3H, m, CH₃).

### Step iv) 4-Ethyl-N,N-dimethyl-3-(1-methyl-1H-imidazo[4,5-c]pyridin-6-ylamino)benzamide

A mixture of 4,5-bis(diphenylphosphino)-9,9-dimethylxanthene (0.08 g), 6-chloro-1-methyl-1H-imidazo[4,5-c]pyridine (0.23 g, 0.0014 mol), 3-amino-4-ethyl-N,N-dimethylbenzamide (0.27 g, 0.0014 mol) and sodium tert-butoxide (0.20 g, 0.0021 mol) in 1,4-dioxane (15 mL) was stirred under N₂ for 20 min. Tris(dibenzylideneacetone)dipalladium(0) (0.06 g) was added and the mixture was stirred at 100° C. for 7 h. The reaction mixture was concentrated in vacuo and the residue was treated with water and extracted into EtOAc. The organics were washed with brine, dried (MgSO₄) and concentrated in vacuo to give the desired compound.

¹H NMR δ (ppm)(CHCl₃-d): 8.69 (1H, d, ArH), 7.71 (1H, s, ArH), 7.52 (1H, d, ArH), 7.32 (1H, m, ArH), 7.16 (1H, dd, ArH), 6.63 (1H, d, ArH), 6.36 (1H, s, NH), 3.68 (3H, s, CH3), 3.09 (3H, s, CH₃), 3.03 (3H, s, CH₃), 2.69 (2H, q, CH₂), 1.24 (3H, t, CH₃).

LCMS (10 cm_ESCI_Formic_MeCN) Rt 2.21 (min) m/z 324 (MH⁺).

### Step v): 4-Ethyl-N,N-dimethyl-3-(methyl(1-methyl-1H-imidazo[4,5-c]pyridin-6-yl)amino)benzamide

4-Ethyl-N,N-dimethyl-3-(1-methyl-1H-imidazo[4,5-c]pyridin-6-ylamino)benzamide (0.3 g, 0.009 mol) was dissolved in DMF (10 mL) and sodium hydride (0.037 g, 60% dispersion in oil, 0.009 mol) was added. The mixture was stirred for 10 min at room temperature before the addition of iodomethane (0.128 g, 0.009 mol) and stirring was continued for 16 h. The mixture was filtered and the filtrate was purified by preparative HPLC to give the desired compound.

¹H NMR δ (ppm)(CHCl₃-d): 8.72 (1H, d, ArH), 7.63 (1H, s, ArH), 7.46-7.37 (2H, m, ArH), 7.29 (1H, dd, ArH), 5.94 (1H, d, ArH), 3.57 (3H, s, CH₃), 3.46 (3H, s, CH₃), 3.07 (6H, m, CH₃), 2.56 (2H, q, CH₂), 1.17 (3H, t, CH₃).

LCMS (10 cm_ESCI_Formic_MeCN) Rt 2.26 (min) m/z 338 (MH⁺).

### Compound 93: N-(2-Ethyl-4-(1-methyl-1H-pyrazol-4-yl)phenyl)-N,1-dimethyl-1H-imidazo[4,5-d]pyridin-6-amine

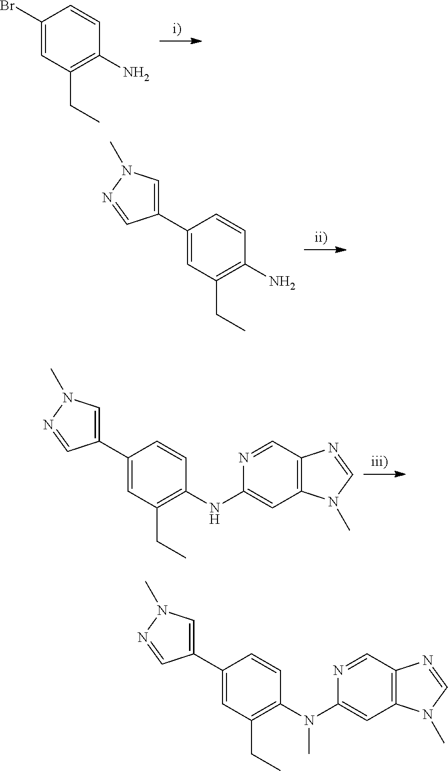

### Step i) 2-Ethyl-4-(1-methyl-1H-pyrazol-4-yl)aniline

4-Bromo-2-ethylaniline (0.5 g, 2.5 mmol), 1-methyl-1H-pyrazol-4-ylboronic acid (0.38 g, 3 mmol) and cesium carbonate (2.4 g, 7.5 mmol) were suspended in 1,4-dioxane (10 mL) and water (2 mL) and the reaction mixture was then degassed (N₂) for 5 min. After which [1,1′-bis(diphenylphosphino)ferrocene]dichloropalladium(II) (0.102 g, 0.125 mmol) was added and the reaction mixture was heated to 95° C. for 1 d. The reaction was cooled to room temperature and filtered through Celite, washed through with DCM. and the organics were washed with water, the layers were separated with the aqueous layer further extracted with DCM. The organics were combined, dried (hydrophobic filter) and concentrated in vacuo. The resulting residue was dissolved in DCM and allowed to load under gravity onto a 10 g SCX column, washed with DCM and MeOH and eluted with 7 N NH₃ solution in MeOH:MeOH (1:5). The eluent was concentrated in vacuo and the resulting residue was purified using column chromatography on silica gel and eluting with 0-75% EtOAc in isohexanes. The fractions containing product were combined and concentrated in vacuo to give the desired compound.

### Step ii) N-(2-Ethyl-4-(1-methyl-1H-pyrazol-4-yl)phenyl)-1-methyl-1H-imidazo[4,5-c]pyridin-6-amine

To stirred degassed (N₂) 1,4-dioxane (10 mL) was added 2-ethyl-4-(1-methyl-1H-pyrazol-4-yl)aniline (400 mg, 2.00 mmol), 6-chloro-1-methyl-1H-imidazo[4,5-c]pyridine (303 mg, 1.8 mmol), tris(dibenzylideneacetone)dipalladium(0) (82 mg, 0.09 mmol), 2-dicyclohexylphosphino-2′,4′,6′-triisopropylbiphenyl (86 mg, 0.18 mmol) and sodium tert-butoxide (260 mg, 2.7 mmol). The reaction mixture was heated to 100° C. for 1 d, cooled to room temperature, filtered through Celite and washed through with DCM. The reaction mixture was washed with water and the layers were separated and the aqueous layer further extracted with DCM. The organics were combined, dried (hydrophobic filter) and concentrated in vacuo and the resulting residue was purified by column chromatography using silica gel and eluting with 0-5% MeOH in DCM. The fractions containing product were combined and concentrated in vacuo to give the desired compound.

¹H NMR δ (ppm)(DMSO-d₆): 8.47 (1H, s, NH), 8.07 (1H, s, ArH), 8.02 (1H, s, ArH), 7.89-7.76 (2H, m, ArH), 7.48-7.40 (2H, m, ArH), 7.37-7.33 (1H, m, ArH), 6.64 (1H, s, ArH), 3.87 (3H, s, CH₃), 3.67 (3H, s, CH₃), 2.70-2.60 (2H, m, CH₂), 1.20-1.11 (3H, m, CH₃).

LCMS (10 cm_ESCI_Bicarb_MeCN) Rt 2.67 (min) m/z 333 (MH⁺).

### Step iii) N-(2-Ethyl-4-(1-methyl-1H-pyrazol-4-yl)phenyl)-N,1-dimethyl-1H-imidazo[4,5-c]pyridin-6-amine

Sodium hydride (60% dispersion in mineral oil, 36 mg, 0.9 mmol) was added to a stirred solution of N-(2-ethyl-4-(1-methyl-1H-pyrazol-4-yl)phenyl)-1-methyl-1H-imidazo[4,5-c]pyridin-6-amine (200 mg, 0.60 mmol) in DMF (3 mL) at 0° C. and allowed to warm to room temperature over 1 h. Iodomethane (42 μL, 0.66 mmol) was added and the reaction mixture was stirred at room temperature for 1 h. The reaction mixture was partitioned between DCM and water, the layers were separated and the aqueous layer further extracted with DCM. The organics were combined and dried (hydrophobic filter), concentrated in vacuo and the resulting residue was purified by preparative HPLC to give the desired compound.

¹H NMR δ (ppm)(DMSO-d₆): 8.49 (1H, s, ArH), 8.17 (1H, s, ArH), 7.97 (1H, s, ArH), 7.89 (1H, s, ArH), 7.58-7.56 (1H, m, ArH), 7.50-7.46 (1H, m, ArH), 7.18-7.12 (1H, m, ArH), 6.14 (1H, s, ArH), 3.88 (3H, s, CH₃), 3.58 (3H, s, CH₃), 3.35 (3H, s, CH₃), 2.50-2.41 (2H, m, CH₂), 1.17-1.08 (3H, m, CH₃).

LCMS (10 cm_ESCI_Bicarb_MeCN) Rt 2.39 (min) m/z 347 (MH⁺).

### Compound 101: 1-(3-Ethyl-4-(methyl(1-methyl-1H-imidazo[4,5-c]pyridin-6-yl)amino)phenyl)ethanol

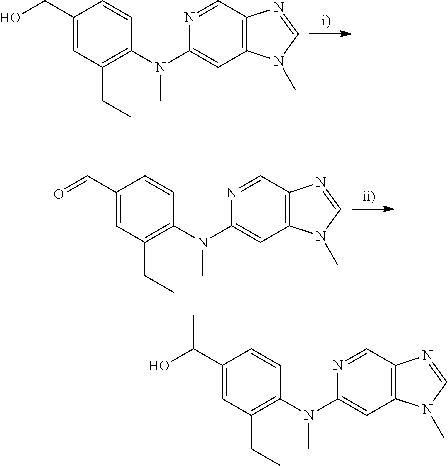

### Step i): 3-Ethyl-4-(methyl(1-methyl-1H-imidazo[4,5-c]pyridin-6-yl)amino)benzaldehyde

(3-Ethyl-4-(methyl(1-methyl-1H-imidazo[4,5-c]pyridin-6-yl)amino)phenyl)MeOH (57 mg, 0.19 mmol) was stirred in DCM (2 mL) at room temperature. Manganese(II) oxide (167 mg, 1.9 mmol) was added and the reaction mixture was stirred at room temperature for 1 d. The reaction mixture was filtered through Celite and the filtrate concentrated in vacuo to give the desired compound which was used in the next step without further purification.

### Step ii): 1-(3-Ethyl-4-(methyl(1-methyl-1H-imidazo[4,5-c]pyridin-6-yl)amino) phenyl)ethanol

3-Ethyl-4-(methyl(1-methyl-1H-imidazo[4,5-c]pyridin-6-yl)amino)benzaldehyde (30 mg, 0.102 mmol) was dissolved in THF (2 mL) and cooled to 0° C. under N₂. Methylmagnesium bromide (0.145 mL, 0.204 mmol, 1.4 M solution in THF) was added slowly and the reaction mixture allowed to warm to room temperature over 1 h. The reaction was quenched with NH₄Cl (saturated aqueous solution) and extracted with EtOAc. The organic layer was washed with brine, dried (MgSO₄), filtered and concentrated in vacuo. The crude material was purified by preparative HPLC to give the desired compound.

¹H NMR δ (ppm)(CHCl₃-d): 8.72 (1H, s, ArH), 7.60 (1H, s, ArH), 7.40 (1H, d, ArH), 7.31 (1H, dd, ArH), 7.19 (1H, d, ArH), 5.89 (1H, s, ArH), 4.97 (1H, q, CH), 3.56 (3H, s, CH₃), 3.44 (3H, s, CH₃), 2.54 (2H, q, CH₂), 1.95 (1H, s, OH), 1.55 (3H, d, CH₃), 1.17 (3H, t, CH₃).

LCMS (10 cm_ESCI_Formic_MeCN) Rt 2.26 (min) m/z 311 (MH⁺).

### Compound 103: 2-(3-Ethyl-4-(methyl(1-methyl-1H-imidazo[4,5-c]pyridin-6-yl)amino)phenyl)propan-2-ol

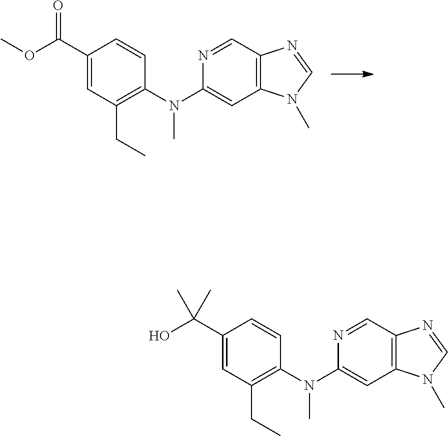 [SMILES: CCc1cc(C(=O)OC)ccc1N(C)c1cc2c(cn1)ncn2C ; CCc1cc(C(C)(C)O)ccc1N(C)c1cc2c(cn1)ncn2C]

Methyl 3-ethyl-4-(methyl(1-methyl-1H-imidazo[4,5-c]pyridin-6-yl)amino)benzoate (53 mg, 0.16 mmol) was dissolved in THF (1 mL) and cooled to 0° C. under N₂. Methylmagnesium bromide (0.408 mL, 0.57 mmol, 1.4 M in THF) was added slowly and the reaction mixture was allowed to warm to room temperature over 1 h. The reaction was quenched with NH₄Cl (saturated aqueous solution) and extracted with EtOAc. The organic layer was washed with brine, dried (MgSO₄), filtered and concentrated in vacuo. The crude material was purified by preparative HPLC to give the desired compound.

¹H NMR δ (ppm)(CHCl₃-d): 8.71 (1H, d, ArH), 7.60 (1H, s, ArH), 7.53 (1H, d, ArH), 7.40 (1H, dd, ArH), 7.16 (1H, d, ArH), 5.89 (1H, d, ArH), 3.56 (3H, s, CH₃), 3.44 (3H, s, CH₃), 2.54 (2H, q, CH₂), 2.01 (1H, s, OH), 1.66 (6H, s, CH₃), 1.17 (3H, t, CH₃).

LCMS (15 cm_Formic_ASCENTIS_HPLC_MeCN) Rt 6.99 (min) m/z 325 (MH⁺).

### Compound 105: N-(2-Ethyl-4-(methoxymethyl)phenyl)-N,1-dimethyl-1H-imidazo[4,5-c]pyridin-6-amine

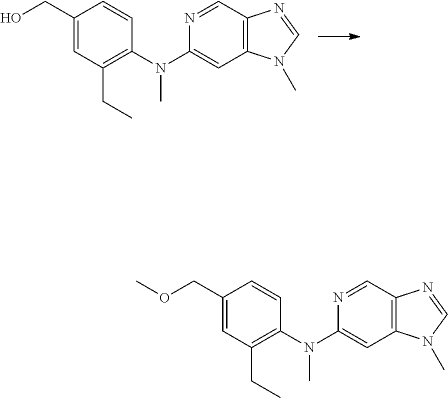 [SMILES: CCc1cc(CO)ccc1N(C)c1cc2c(cn1)ncn2C ; CCc1cc(COC)ccc1N(C)c1cc2c(cn1)ncn2C]

(3-Ethyl-4-(methyl(1-methyl-1H-imidazo[4,5-c]pyridin-6-yl)amino)phenyl)MeOH (33 mg, 0.11 mmol) was dissolved in DMF (2 mL) and cooled to 0° C. under N₂. At this point, sodium hydride (60% dispersion in mineral oil, 7 mg, 0.167 mmol) was added and the reaction mixture was allowed to warm to room temperature and stirred for 1 h. Iodomethane (10 mL, 7 mg, 0.167 mmol) was added and the reaction mixture was allowedrt for 1 d. The reaction was quenched with NH₄Cl (saturated aqueous solution) and extracted with EtOAc. The organic layer was washed with brine, dried (MgSO₄), filtered and concentrated in vacuo. The crude material was purified by preparative HPLC to give the desired compound.

¹H NMR δ (ppm)(CHCl₃-d): 8.73 (1H, d, ArH), 7.60 (1H, s, ArH), 7.38 (1H, d, ArH), 7.30-7.25 (1H, m, ArH), 7.19 (1H, d, ArH), 5.88 (1H, s, ArH), 4.49 (2H, s, CH₂), 3.54 (3H, s, CH₃), 3.50 (3H, s, CH₃), 3.44 (3H, s, CH₃), 2.54 (2H, q, CH₂), 1.16 (3H, t, CH₃).

LCMS (15 cm_Bicarb_GeminiNX_HPLC_MeCN) Rt 9.29 (min) m/z 311 (MH⁺).

### Compound 109: (4-Ethyl-3-(methyl(1-methyl-1H-imidazo[4,5-c]pyridin-6-yl)amino)phenyl)MeOH

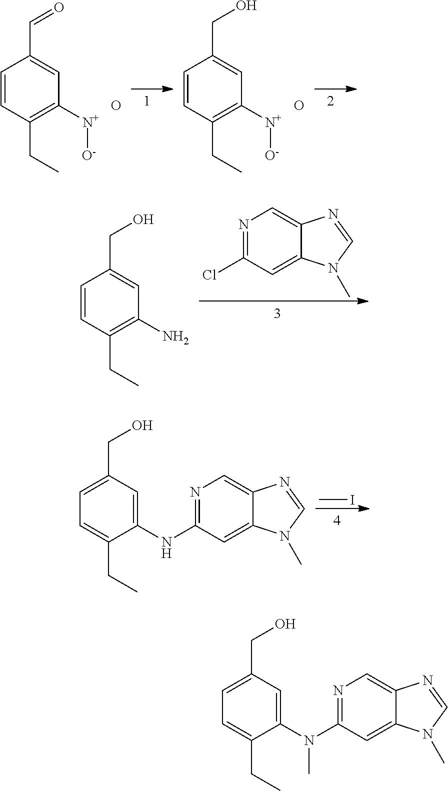

### Step 1: (4-Ethyl-3-nitrophenyl)MeOH

A solution of 4-ethyl-3-nitrobenzaldehyde (1.07 g, 0.006 mol) in MeOH (15 mL) was treated with sodium borohydride (0.25 g, 0.0066 mol) and the resulting solution was stirred at room temperature for 1.5 h. The mixture was concentrated in vacuo and the residue was treated with water (20 mL) and extracted with DCM. The organics were dried (MgSO₄), filtered and concentrated in vacuo to give the desired compound.

¹H NMR δ (ppm)(CHCl₃-d): 7.98-7.88 (1H, m, ArH), 7.61-7.52 (1H, m, ArH), 7.38 (1H, d, ArH), 4.77 (2H, d, CH2), 2.93 (2H, q, CH2), 1.90 (1H, t, OH), 1.38-1.26 (3H, m, CH3).

### Step 2: (3-Amino-4-ethylphenyl)MeOH

Palladium-on-carbon (10% wt., 1.4 g) was added to a stirred solution of (4-ethyl-3-nitrophenyl)MeOH (1.05 g, 0.0058 mol) and ammonium formate (2.9 g, 0.046 mol) in MeOH (20 mL) under N2. The mixture was gently refluxed for 8 h before filtering through Celite and concentrating in vacuo. The residue was triturated with water (5 mL), filtered and dried to give the desired compound.

¹H NMR δ (ppm)(DMSO-d₆): 6.93-6.81 (1H, m, ArH), 6.58 (1H, s, ArH), 6.53-6.38 (1H, m, ArH), 4.90 (1H, s, OH), 4.74 (2H, s, NH2), 4.32 (2H, s, CH2), 2.41 (2H, q, CH2), 1.17-1.04 (3H, m, CH3).

### Step 3: (4-Ethyl-3-(1-methyl-1H-imidazo[4,5-c]pyridin-6-ylamino)phenyl)MeOH

To stirred degassed (N2) 1,4-dioxane (30 mL) was added (3-amino-4-ethylphenyl)MeOH (440 mg, 2.90 mmol), 6-chloro-1-methyl-1H-imidazo[4,5-c]pyridine (480 mg, 2.9 mmol), tris(dibenzylideneacetone)dipalladium(0) (0.12 mg, 0.145 mmol), 4,5-bis(diphenylphosphino)-9,9-dimethylxanthene (160 mg, 0.29 mmol) and sodium tert-butoxide (690 mg, 0.725 mmol). The reaction mixture was heated to 100° C. for 7 h, cooled to room temperature, concentrated in vacuo and ethyl acetate and water were added. The ethyl acetate was separated, dried (MgSO₄), filtered and concentrated in vacuo. The residue was dissolved in MeOH and loaded onto a 20 g SCX cartridge which was washed with MeOH and eluted with dilute ammonia in MeOH to give the desired compound.

### Step 4: (4-Ethyl-3-(methyl(1-methyl-1H-imidazo[4,5-c]pyridin-6-yl)amino)phenyl)MeOH

Sodium hydride (60% in oil, 0.0368 g, 0.92 mmol) was added to a stirred solution of (4-ethyl-3-(1-methyl-1H-imidazo[4,5-c]pyridin-6-ylamino)phenyl)MeOH (260 mg, 0.92 mmol) in dry DMF (2.5 mL). After stirring at room temperature for 10 min, iodomethane (130 mg, 0.92 mmol) was added and stirring was continued for 4 h at room temperature. The reaction mixture was filtered and the filtrate was purified by preparative HPLC to give the desired compound.

¹H NMR δ (ppm)(CHCl₃-d): 8.71 (1H, s, ArH), 7.60 (1H, s, ArH), 7.39 (1H, d, ArH), 7.33 (1H, d, ArH), 7.21 (1H, s, ArH), 5.89 (1H, s, ArH), 4.70 (2H, s, CH2), 3.55 (3H, s, CH3), 3.45 (3H, s, CH3), 2.52 (2H, q, CH2), 1.99 (1H, m, OH), 1.15 (3H, t, CH3).

LCMS (15 cm_Formic_ASCENTIS_HPLC_MeCN) Rt 6.87 (min) m/z 297 (MH+).

### Compound 110: N-(2-Ethyl-4-((1-(methylsulfonyl)pyrrolidin-3-yl)oxy)phenyl)-N,1-dimethyl-1H-imidazo[4,5-c]pyridin-6-amine

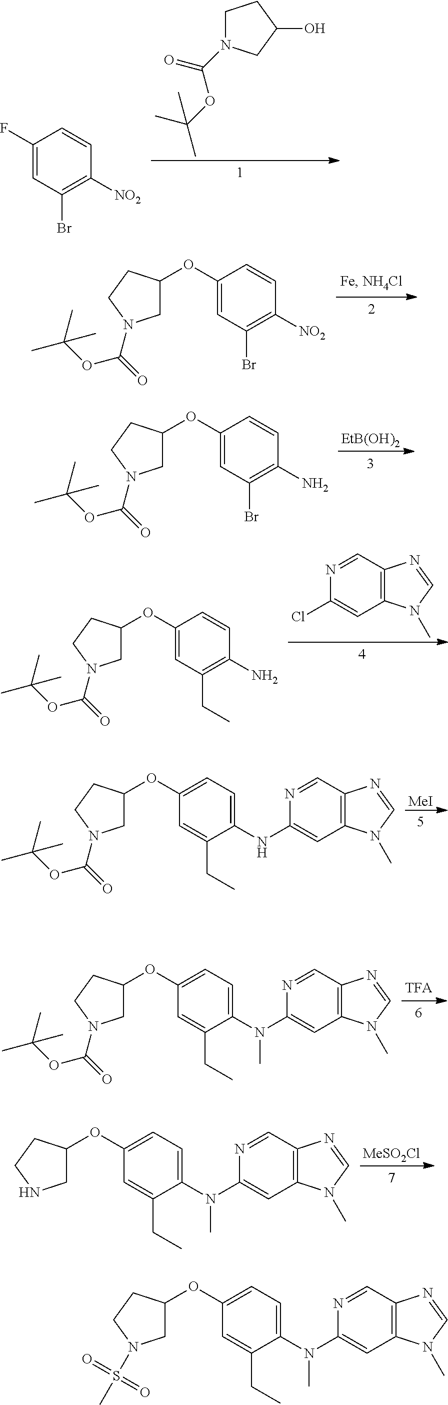

### Step 1: tert-Butyl 3-(3-bromo-4-nitrophenoxy)pyrrolidine-1-carboxylate

1-Bromo-5-fluoro-2-nitrobenzene (1 g, 4.54 mmol), tert-butyl 3-hydroxypyrrolidine-1-carboxylate (1.02 g, 5.45 mmol) and tetrabutylammonium hydrogen sulfate (154 mg, 0.45 mmol) were stirred vigorously in NaOH (1 M aqueous solution, 20 mL) and DCM (20 mL) for 1 d. The layers were separated and the aqueous was extracted with DCM. The combined organics were dried (hydrophobic frit) and concentrated in vacuo. The residue was purified by column chromatography using silica gel and eluting with 0-65% EtOAc in isohexanes to give the desired compound

### Step 2: tert-Butyl 3-(4-amino-3-bromophenoxy)pyrrolidine-1-carboxylate

The nitro-benzene (2.7 mmol), NH₄Cl (217 mg, 4.05 mmol) and iron powder (753 mg, 13.5 mmol) were suspended in THF (3 mL), ethanol (3 mL) and water (1 mL). The stirred reaction mixture was heated to 90° C. for 1 h. The reaction mixture was filtered through Celite, washed through with DCM and concentrated in vacuo. The resulting residue was partitioned between DCM and sat. aq. NaHCO₃ solution. The layers were separated and the aqueous layer extracted with DCM, the organics were combined and dried (hydrophobic filter) and concentrated in vacuo. The resulting residue was purified using column chromatography on silica gel to give the desired compound.

### Step 3: tert-Butyl 3-(4-amino-3-ethylphenoxy)pyrrolidine-1-carboxylate

tert-Butyl 3-(4-amino-3-bromophenoxy)pyrrolidine-1-carboxylate (983 mg, 2.75 mmol), ethyl boronic acid (245 mg, 3.3 mmol), [1,1′-bis(diphenylphosphino)ferrocene]dichloropalladium(II) (112 mg, 0.14 mmol) and K₃PO₄ (1.75 g, 8.25 mmol) were heated in 1,4-dioxane (20 mL) at reflux for 1 d. The reaction mixture was cooled to room temperature and filtered through Celite, washed through with DCM. and the organics were washed with water, dried (hydrophobic frit) and concentrated in vacuo. The residue was loaded onto a SCX column in DCM. The column was washed with MeOH and DCM and eluted with 7 N NH₃ in MeOH:MeOH (1:5). The eluent was concentrated in vacuo. The residue was purified by column chromatography using silica gel and eluting with 0-75% EtOAc in isohexanes to give the desired compound.

### Step 4: tert-Butyl 3-(3-ethyl-4-((1-methyl-1H-imidazo[4,5-c]pyridin-6-yl)amino)phenoxy)pyrrolidine-1-carboxylate

Prepared using method A to give the desired compound.

¹H NMR δ (ppm)(DMSO-d₆): 8.42 (1H, d, ArH), 7.97 (1H, s, NH), 7.73 (1H, s, ArH), 7.27 (1H, d, ArH), 6.84 (1H, d, ArH), 6.78 (1H, dd, ArH), 6.39 (1H, s, ArH), 4.98 (1H, s, CH), 3.64 (3H, s, CH3), 3.60-3.32 (4H, m, CH), 2.57 (2H, q, CH2), 2.19-2.02 (2H, s, CH), 1.41 (9H, s, CH3), 1.10 (3H, t, CH3).

### Step 5: tert-Butyl 3-(3-ethyl-4-(methyl(1-methyl-1H-imidazo[4,5-c]pyridin-6-yl)amino)phenoxy)pyrrolidine-1-carboxylate

Prepared using method B to give the desired compound.

### Step 6: N-(2-Ethyl-4-(pyrrolidin-3-yloxy)phenyl)-N,1-dimethyl-1H-imidazo[4,5-c]pyridin-6-amine

tert-Butyl 3-(3-ethyl-4-(methyl(1-methyl-1H-imidazo[4,5-c]pyridin-6-yl)amino)phenoxy)pyrrolidine-1-carboxylate (120 mg, 0.27 mmol) was stirred in DCM (3 mL) and trifluoroacetic acid (0.3 mL) at room temperature for 1 h. The reaction mixture was concentrated in vacuo, dissolved in DCM and loaded onto a SCX column. The column was washed with MeOH and DCM and eluted with 7 N NH₃ in MeOH:MeOH (1:5) to give the desired compound.

### Step 7N-(2-Ethyl-4-O-(methylsulfonyl)pyrrolidin-3-yl)oxy)phenyl)-N,1-dimethyl-1H-imidazo[4,5-c]pyridin-6-amine

Methylsulfonyl chloride (20 μL, 0.25 mmol) was added to a solution of N-(2-ethyl-4-(pyrrolidin-3-yloxy)phenyl)-N,1-dimethyl-1H-imidazo[4,5-c]pyridin-6-amine (80 mg, 0.23 mmol) and triethylamine (0.39 mL, 0.28 mmol) in DCM (3 mL) at 0° C. The reaction mixture was allowed to warm to room temperature over 1 d. The reaction mixture was concentrated in vacuo and purified by preparative HPLC to give the desired compound.

¹H NMR δ (ppm)(DMSO-d₆): 8.48 (1H, s, ArH), 7.96 (1H, s, ArH), 7.11 (1H, d, ArH), 6.95 (1H, d, ArH), 6.91-6.86 (1H, m, ArH), 6.06 (1H, s, ArH), 5.09 (1H, s, CH), 3.66-3.59 (1H, m, CH), 3.58 (3H, s, CH3), 3.32 (3H, s, CH3), 2.99-2.87 (3H, m, CH), 2.46-2.36 (2H, m, CH), 2.31-2.20 (1H, m, CH), 2.19-2.11 (1H, m, CH), 1.15-1.05 (3H, m, CH3), CH3 under water peak).

LCMS (15 cm_Formic_ASCENTIS_HPLC_MeCN) Rt 7.23 (min) m/z 430 (MH+).

### Compound 115: N-(5-(3-Ethyl-4-(methyl(1-methyl-1H-imidazo[4,5-c]pyridin-6-yl)amino)phenyl)pyrimidin-2-yl)acetamide

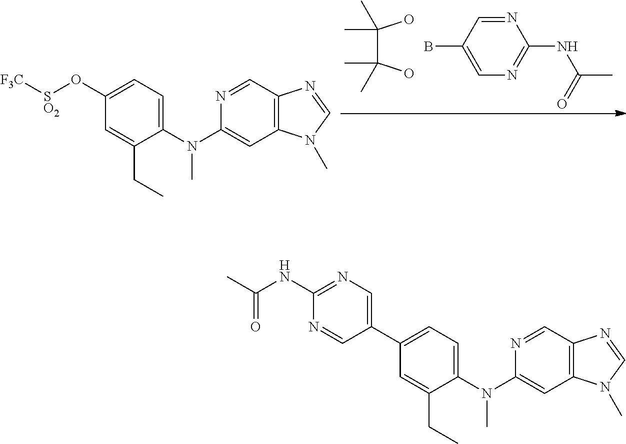

3-Ethyl-4-(methyl(1-methyl-1H-imidazo[4,5-c]pyridin-6-yl)amino)phenyl trifluoromethanesulfonate (129 mg, 0.31 mmol), 2-acetamidopyrimidine-5-boronic acid pinacol ester (98 mg, 0.37 mmol), [1,1′-bis(diphenylphosphino)ferrocene]dichloropalladium(II) (13 mg, 0.016 mmol) and cesium carbonate (303 mg, 0.93 mmol) were heated at reflux in 1,4-dioxane (3 mL) and water (1 mL) for 2 h. The reaction mixture was cooled to room temperature, filtered through Celite and washed through with DCM. The organics were washed with water, dried (hydrophobic frit), concentrated in vacuo and purified by preparative HPLC to give the desired compound.

¹H NMR δ (ppm)(DMSO-d₆): 10.65 (1H, s, NH), 9.03 (2H, s, ArH), 8.48 (1H, d, ArH), 8.01-7.94 (1H, m, ArH), 7.76 (1H, d, ArH), 7.67 (1H, dd, ArH), 7.29 (1H, d, ArH), 6.28 (1H, s, ArH), 3.62 (3H, s, CH3), 3.36 (3H, s, CH3), 2.53-2.44 (2H, m, CH2), 2.21 (3H, s, CH3), 1.17-1.08 (3H, m, CH3).

LCMS (15 cm_Bicarb_GeminiNX_HPLC_MeCN) Rt 8.28 (min) m/z 402 (MH+).

### Compound 116: 4-Ethyl-N-(2-methoxyethyl)-3-(methyl(1-methyl-1H-imidazo[4,5-c]pyridin-6-yl)amino)benzamide

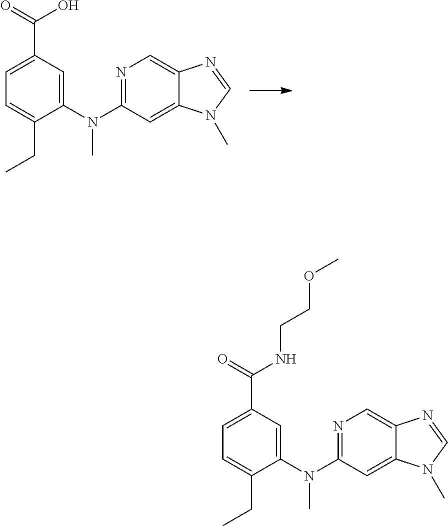 [SMILES: CCc1ccc(C(=O)O)cc1N(C)c1cc2c(cn1)ncn2C ; CCc1ccc(C(=O)NCCOC)cc1N(C)c1cc2c(cn1)ncn2C]

(2-(7-Aza-1H-benzotriazole-1-yl)-1,1,3,3-tetramethyluronium hexafluorophosphate) (0.084 g, 0.22 mmol) was added to a stirred solution 4-ethyl-3-(methyl(1-methyl-1H-imidazo[4,5-c]pyridin-6-yl)amino)benzoic acid (0.062 g, 0.2 mmol) and triethylamine (0.06 g, 0.6 mmol) in DMF (1 mL) and the resulting solution was stirred at room temperature for 30 min. 2-Methoxyethanamine (0.019 g, 0.4 mmol) was added and the reaction was stirred at room temperature for 1 d. The reaction mixture was filtered and the filtrate was purified by preparative HPLC to give the desired compound.

¹H NMR δ (ppm)(CHCl₃-d): 8.65 (1H, s, ArH), 7.75-7.64 (3H, m, ArH), 7.45 (1H, d, ArH), 6.59 (1H, m, NH), 6.00 (1H, s, ArH), 3.67-3.59 (5H, m, CH2, CH3), 3.56 (2H, t, CH2), 3.59-3.37 (3H, m, CH3), 3.37 (3H, s, CH3), 2.55 (2H, q, CH2), 1.17 (3H, t, CH3).

LCMS (15 cm_Bicarb_GeminiNX_HPLC_MeCN) Rt 8.38 (min) m/z 368 (MH+).

### Compound 122: N-(2-Ethyl-5-(methoxymethyl)phenyl)-N,1-dimethyl-1H-imidazo[4,5-c]pyridin-6-amine

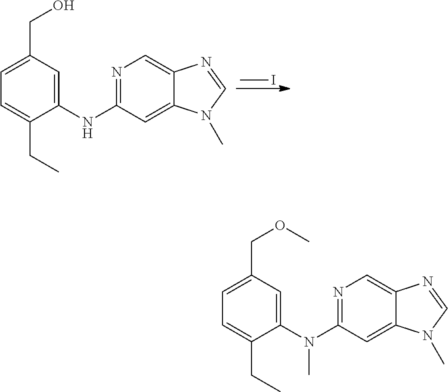

Sodium hydride (60% in oil, 0.097 g, 2.43 mmol) was added to a stirred solution of (4-ethyl-3-(1-methyl-1H-imidazo[4,5-c]pyridin-6-ylamino)phenyl)MeOH (230 mg, 0.81 mmol) in dry DMF (2.5 mL) at room temperature. After stirring for 10 min, iodomethane (340 mg, 2.43 mmol) was added and stirring was continued for 4 h at room temperature. The reaction mixture was filtered and the filtrate was purified by preparative HPLC to give the desired compound.

¹H NMR δ (ppm)(DMSO-d₆): 8.49 (1H, d, ArH), 7.98 (1H, s, ArH), 7.37 (1H, d, ArH), 7.24 (1H, dd, ArH), 7.12 (1H, d, ArH), 6.13 (1H, d, ArH), 4.39 (2H, s, CH2), 3.59 (3H, s, CH3), 3.39-3.24 (5H, m, CH2, CH3), 2.54-2.41 (3H, m, CH3), 1.09 (3H, t, CH3).

LCMS (10 cm_ESCI_Formic_MeCN) Rt 2.39 (min) m/z 311 (MH+).

### Compound 132: 5-(3-Ethyl-4-(methyl(1-methyl-1H-imidazo[4,5-c]pyridin-6-yl)amino)phenyl)pyridin-2-ol

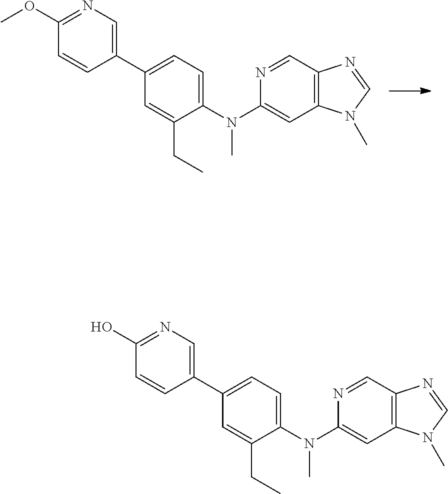 [SMILES: CCc1cc(-c2ccc(OC)nc2)ccc1N(C)c1cc2c(cn1)ncn2C ; CCc1cc(-c2ccc(O)nc2)ccc1N(C)c1cc2c(cn1)ncn2C]

Boron tribromide (2.7 mmol, 0.26 mL) was added to a solution of N-(2-ethyl-4-(6-methoxypyridin-3-yl)phenyl)-N,1-dimethyl-1H-imidazo[4,5-c]pyridin-6-amine (100 mg, 0.27 mmol) in DCM (5 mL) at 78° C. under an atmosphere of nitrogen. The reaction mixture was allowed to warm to room temperature over 1 d. The reaction mixture was cooled to 78° C. and quenched with sat. aqueous K₂CO₃ solution and diluted with DCM. A solid precipitate was filtered off and purified by preparative HPLC to give the desired compound.

¹H NMR δ (ppm)(DMSO-d₆): 8.49-8.48 (1H, m, ArH), 7.98 (1H, s, ArH), 7.92-7.87 (1H, m, ArH), 7.78-7.76 (1H, m, ArH), 7.57-7.55 (1H, m, ArH), 7.51-7.47 (1H, m, ArH), 7.22-7.18 (1H, m, ArH), 6.47-6.42 (1H, m, ArH), 6.20 (1H, s, ArH), 3.61 (3H, s, CH3), 2.50-2.43 (2H, m, CH2), 1.17-1.08 (3H, m, CH3), (CH3 under water peak), (OH not observed).

LCMS (10 cm_Formic_ACE 3 C18 AR_HPLC_MeCN) Rt 6.93 (min) m/z 360 (MH+).

### Compound 134: 5-(3-Ethyl-4-(methyl(1-methyl-1H-imidazo[4,5-c]pyridin-6-yl)amino)phenyl)pyrimidin-2-ol

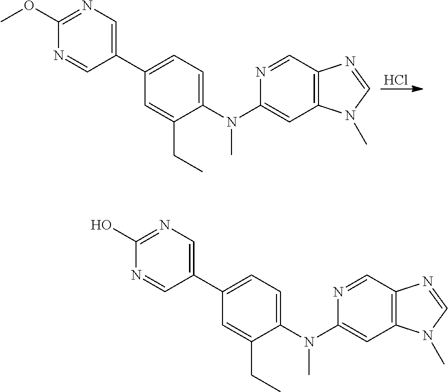 [SMILES: CCc1cc(-c2cnc(OC)nc2)ccc1N(C)c1cc2c(cn1)ncn2C ; Cl ; CCc1cc(-c2cnc(O)nc2)ccc1N(C)c1cc2c(cn1)ncn2C]

A stirred solution of N-(2-ethyl-4-(2-methoxypyrimidin-5-yl)phenyl)-N,1-dimethyl-1H-imidazo[4,5-c]pyridin-6-amine (55 mg, 0.15 mmol) in MeOH (3 mL), containing conc. HCl (4 drops), was heated to reflux for 6 d. The reaction mixture was cooled to room temperature and DCM and water were added. The pH was adjusted to pH 7 using NaOH (1 M, aqueous) and the aqueous layer was extracted with DCM. The organics were dried (hydrophobic frit), concentrated in vacuo and purified by preparative HPLC to give the desired compound.

¹H NMR δ (ppm)(DMSO-d₆): 12.22 (1H, s, OH), 8.68 (2H, s, ArH), 8.47 (1H, d, ArH), 7.98 (1H, s, ArH), 7.63 (1H, d, ArH), 7.54 (1H, dd, ArH), 7.22 (1H, d, ArH), 6.22 (1H, s, ArH), 3.60 (3H, s, CH3), 3.35 (3H, s, CH3), 2.52-2.44 (2H, m, CH2), 1.12 (3H, t, CH3).

LCMS (10 cm_ESCI_Formic_MeCN) Rt 2.05 (min) m/z 361 (MH+).

### Compound 136: N-(2-Ethyl-4-((4-(methylsulfonyl)piperazin-1-yl)methyl)phenyl)-N,1-dimethyl-1H-imidazo[4,5-c]pyridin-6-amine

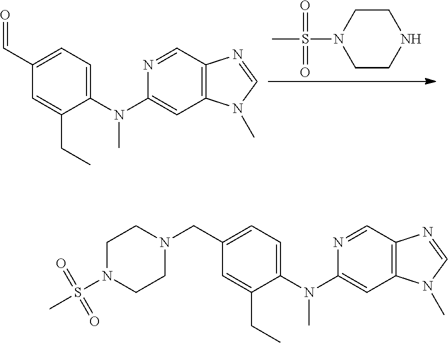

3-Ethyl-4-(methyl(1-methyl-1H-imidazo[4,5-c]pyridin-6-yl)amino)benzaldehyde (100 mg, 0.34 mmol), 1-methylsulfonylpiperizine (167 mg, 1.02 mmol) and (polystyrylmethyl)trimethylammoniumcyanoborohydride (4 mmol/g loading, 250 mg, 1.02 mmol) were shaken in DCM (3 mL) and acetic acid (0.3 mL) for 1 d. The beads were filtered off and the filtrate was concentrated in vacuo and purified by preparative HPLC to give the desired compound

¹H NMR δ (ppm)(DMSO-d₆): 8.47 (1H, d, ArH), 7.96 (1H, s, ArH), 7.30 (1H, d, ArH), 7.23 (1H, dd, ArH), 7.12 (1H, d, ArH), 6.10 (1H, s, ArH), 3.59 (3H, s, CH3), 3.57 (2H, s, CH2), 3.33 (3H, s, CH3), 3.15-3.11 (4H, m, CH), 2.87 (3H, s, CH3), 2.52-2.39 (6H, m, CH2, CH), 1.07 (3H, t, CH3).

LCMS (10 cm_ESCI_Bicarb_MeCN) Rt 3.18 (min) m/z 443 (MH+).

### Compound 137: 3-Ethyl-4-(methyl(1-methyl-1H-imidazo[4,5-c]pyridin-6-yl)amino)benzamide

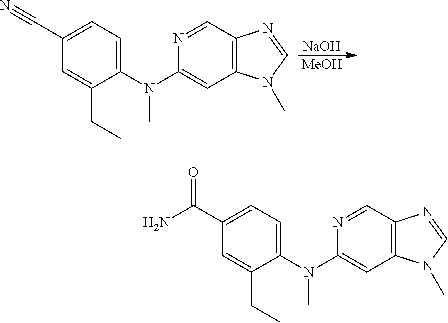

3-Ethyl-4-(methyl(1-methyl-1H-imidazo[4,5-c]pyridin-6-yl)amino)benzonitrile (100 mg, 0.34 mmol) was heated in NaOH (1 M, aqueous, 2 mL) and MeOH (2 mL) at 45° C. for 2 d. The reaction mixture was cooled to room temperature and DCM and water were added. The aqueous layer was extracted with DCM and the combined organics were washed with water, dried (hydrophobic filter), concentrated in vacuo and purified by preparative HPLC to give the desired compound.

¹H NMR δ (ppm)(DMSO-d₆): 8.47 (1H, d, ArH), 8.01-7.94 (2H, m, ArH, NH), 7.88 (1H, d, ArH), 7.78 (1H, dd, ArH), 7.33 (1H, s, NH), 7.23 (1H, d, ArH), 6.24 (1H, d, ArH), 3.60 (3H, s, CH3), 3.34 (3H, s, CH3), 2.52-2.42 (2H, m, CH2), 1.10 (3H, t, CH3).

LCMS (10 cm_ESCI_Bicarb_MeCN) Rt 2.62 (min) m/z 310 (MH+).

### Compound 151: N-(2-Ethyl-4-(thiazol-5-yl)phenyl)-N,1-dimethyl-1H-imidazo[4,5-c]pyridin-6-amine

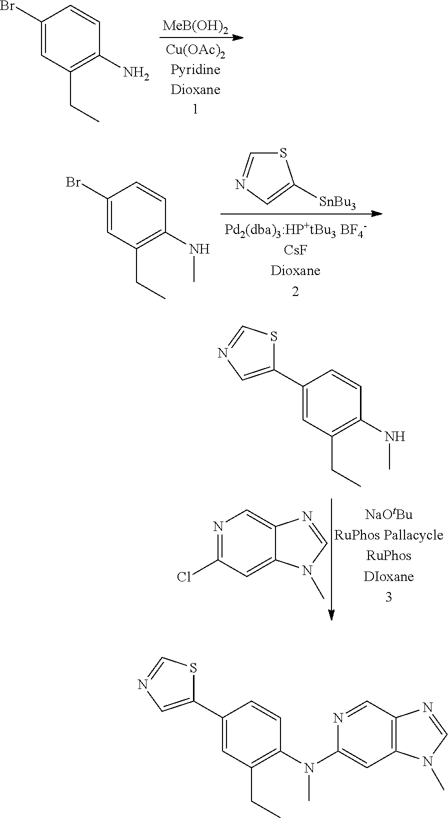

### Step 1: 4-Bromo-2-ethyl-N-methylaniline

A mixture of 2-ethyl-4-bromoaniline (2.84 mL, 20 mmol), copper(II) acetate (3.0 g, 50 mmol) and pyridine (5.66 mL, 70 mmol) in 1,4-dioxane (240 mL) were stirred at room temperature for 15 min. Methylboronic acid (3.0 g, 50 mmol) was added and the resulting mixture was heated to reflux for 48 h. The mixture was cooled to room temperature, filtered through Celite and concentrated in vacuo. Purification by silica chromatography (eluent, 10% EtOAc:iso-hexane) gave the desired compound.

### Step 2: 2-Ethyl-N-methyl-4-(thiazol-5-yl)aniline

A stirred mixture of 4-bromo-2-ethyl-N-methylaniline (0.520 g, 2.42 mmol), 5-(tributylstannyl)thiazole (1.0 g, 2.66 mmol), cesium fluoride (0.811 g, 5.32 mmol) and tris(dibenzylideneacetone)dipalladium/tri-tert-butyl phosphonium tetrafluoroborate mixture (mole ratio: 1/1.2) (0.158 g, 0.12 mmol) in 1,4-dioxane (5 mL) was degassed under N2(g) for 10 min. The reaction mixture was heated to reflux for 24 h, cooled to room temperature and filtered through Celite. After concentrating in vacuo the resulting residue was dissolved in DMSO (1 mL) and purified by preparative HPLC to give the desired compound.

### Step 3: N-(2-Ethyl-4-(thiazol-5-yl)phenyl)-N,1-dimethyl-1H-imidazo[4,5-c]pyridin-6-amine

At room temperature, 6-chloro-1-methyl-1H-imidazo[4,5-c]pyridine (0.135 g, 0.80 mmol) was stirred with 2-dicyclohexylphosphino-2′,4′,6′-triisopropylbiphenyl (0.017 g, 0.0365 mmol), chloro (2-dicyclohexylphosphino-2′,4′,6′-triisopropyl-1,1′-biphenyl) [2-(2-aminoethyl)phenyl]palladium(II) (0.027 g, 0.0365 mmol), sodium tert-butoxide (0.105 g, 1.10 mmol) and 2-ethyl-N-methyl-4-(thiazol-5-yl)aniline (0.160 g, 0.73 mmol) in 1,4-dioxane (5 mL) and degassed under N₂ (g) for 10 min. At this point the reaction was stirred at 100° C. for 8 h. The mixture was cooled to room temperature and filtered through Celite and concentrated in vacuo. The resulting residue was dissolved in DMSO (1 mL) and purified by preparative HPLC to give the desired compound.

¹H NMR δ (ppm)(DMSO-d₆): 9.09 (1H, d, ArH), 8.47 (1H, d, ArH), 8.35-8.33 (1H, m, ArH), 7.98 (1H, s, ArH), 7.67 (1H, d, ArH), 7.60 (1H, dd, ArH), 7.24 (1H, d, ArH), 6.26 (1H, d, ArH), 3.61 (3H, s, CH3), 2.48-2.42 (2H, m, CH2), 1.12 (3H, t, CH3), (CH3 under water peak).

LCMS (10 cm_ESCI_Formic_MeCN) Rt 2.42 (min) m/z 350 (MH+).

### Compound 156: 3-(3-(3-Ethyl-4-(methyl(1-methyl-1H-imidazo[4,5-c]pyridin-6-yl)amino)phenyl)-1,2,4-oxadiazol-5-yl)propanenitrile

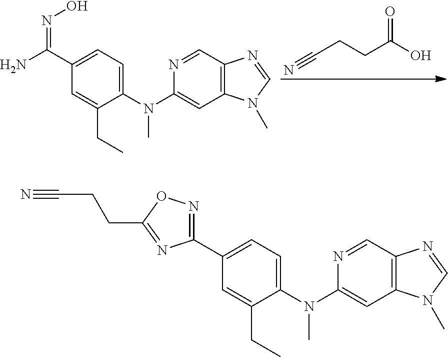

3-Ethyl-N′-hydroxy-4-(methyl(1-methyl-1H-imidazo[4,5-c]pyridin-6-yl)amino)benzimidamide (100 mg, 0.31 mmol), 3-cyanopropanoic acid (34 mg, 0.34 mmol), (2-(7-aza-1H-benzotriazole-1-yl)-1,1,3,3-tetramethyluronium hexafluorophosphate) (177 mg, 0.47 mmol) and diisopropylethylamine (0.22 mL, 1.24 mmol) were stirred in DMF (3 mL) at 55° C. for 3 d. The crude reaction mixture was purified by preparative HPLC to give the desired compound.

¹H NMR δ (ppm)(DMSO-d₆): 8.48 (1H, d, ArH), 8.00-7.97 (2H, m, ArH), 7.92 (1H, dd, ArH), 7.38 (1H, d, ArH), 6.33 (1H, d, ArH), 3.62 (3H, s, CH3), 3.45-3.40 (2H, m, CH), 3.31 (3H, s, CH3), 3.13-3.05 (2H, m, CH), 2.55-2.48 (2H, m, CH2), 1.12 (3H, t, CH3).

LCMS (15 cm_Formic_ASCENTIS_HPLC_MeCN) Rt 7.45 (min) m/z 388 (MH⁺).

### Compound 159: 1-(3-Ethyl-4-(methyl(1-methyl-1H-imidazo[4,5-c]pyridin-6-yl)amino)phenyl)-2,2,2-trifluoroethanol

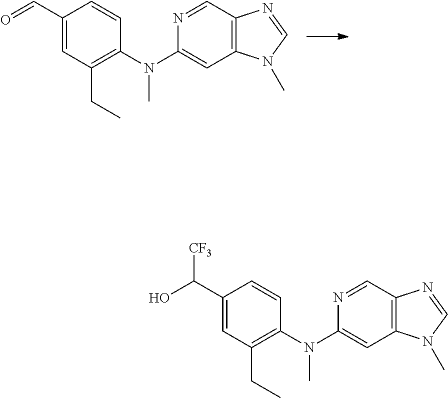 [SMILES: CCc1cc(C=O)ccc1N(C)c1cc2c(cn1)ncn2C ; CCc1cc(C(O)C(F)(F)F)ccc1N(C)c1cc2c(cn1)ncn2C]

Tetrabutylammonium fluoride (1 drop, cat.) was added to a solution of 3-ethyl-4-(methyl(1-methyl-1H-imidazo[4,5-c]pyridin-6-yl)amino)benzaldehyde (75 mg, 0.26 mmol) and trimethyl(trifluoromethyl)silane (40 mg, 0.31 mmol) in tetrahydrofuran (5 mL). The reaction was stirred at room temperature for 1 h and trimethyl(trifluoromethyl)silane (80 mg, 0.612 mmol) was added. The reaction was stirred for a further 3 h at room temperature then quenched by the addition of aqueous HCl solution (4 drops, 2 M). The reaction was concentrated in vacuo and purified by preparative HPLC to give the desired compound.

¹H NMR δ (ppm)(DMSO-d₆): 8.47 (1H, s, ArH), 7.99 (1H, s, ArH), 7.50 (1H, s, ArH), 7.41 (1H, d, ArH), 7.21 (1H, d, ArH), 6.84 (1H, s, OH), 6.23 (1H, s, ArH), 5.19 (1H, m, CH), 3.62 (3H, s, CH3), 3.33 (3H, s, CH3), 2.46 (2H, q, CH2), 1.09 (3H, t, CH3).

LCMS (10 cm_ESCI_Bicarb_MeCN) Rt 2.86 (min) m/z 365 (MH⁺).

### Compound 173: N-(2-Ethyl-4-((2-methyl-1H-imidazol-1-yl)methyl)phenyl)-N,1-dimethyl-1H-imidazo[4,5-c]pyridin-6-amine

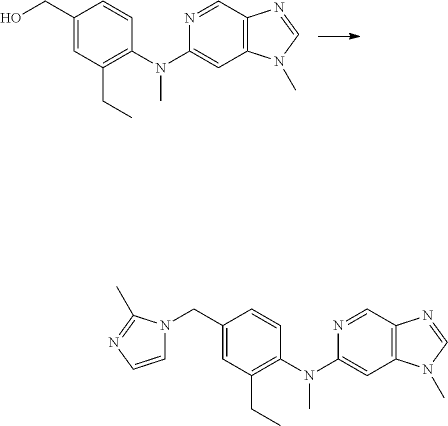 [SMILES: CCc1cc(CO)ccc1N(C)c1cc2c(cn1)ncn2C ; CCc1cc(Cn2ccnc2C)ccc1N(C)c1cc2c(cn1)ncn2C]

(3-Ethyl-4-(methyl(1-methyl-1H-imidazo[4,5-c]pyridin-6-yl)amino)phenyl)MeOH (40 mg, 0.14 mmol) was dissolved in DMF (2 mL). Bis-(2-methyl-1H-imidazol-1-yl)methanone (128 mg, 0.68 mmol) was added and the reaction mixture was heated to 100° C. for 5 h. The reaction was allowed to cool and the crude material was purified by preparative HPLC to give the desired compound.

¹H NMR δ (ppm)(CHCl₃-d): 8.71 (1H, s, ArH), 7.62 (1H, s, ArH), 7.17 (1H, d, ArH), 7.10 (1H, s, ArH), 7.03-6.95 (2H, m, ArH), 6.93 (1H, s, ArH), 5.89 (1H, s, ArH), 5.08 (2H, s, CH2), 3.58 (3H, s, CH3), 3.42 (3H, s, CH3), 2.50 (2H, q, CH2), 2.41 (3H, s, CH3), 1.11 (3H, t, CH3).

LCMS (15 cm_Bicarb_GeminiNX_HPLC_MeCN) Rt 8.55 (min) m/z 361 (MH+).

### Compound 175: 3-Ethyl-4-(methyl(1-methyl-1H-imidazo[4,5-c]pyridin-6-yl)amino)-N-(1-(methylsulfonyl)azetidin-3-yl)benzamide

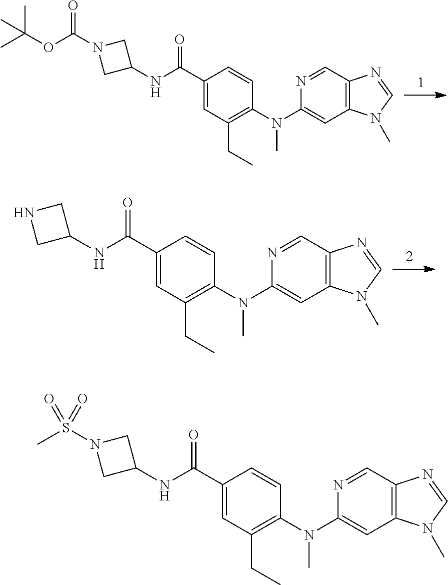 [SMILES: CCc1cc(C(=O)NC2CN(C(=O)OC(C)(C)C)C2)ccc1N(C)c1cc2c(cn1)ncn2C ; CCc1cc(C(=O)NC2CNC2)ccc1N(C)c1cc2c(cn1)ncn2C ; CCc1cc(C(=O)NC2CN(S(C)(=O)=O)C2)ccc1N(C)c1cc2c(cn1)ncn2C]

### Step 1: N-(Azetidin-3-yl)-3-ethyl-4-(methyl(1-methyl-1H-imidazo[4,5-c]pyridin-6-yl)amino)benzamide

A solution of trifluoroacetic acid:DCM (1:1, 10 mL) containing water (3 drops) was added dropwise to tert-butyl 3-(3-ethyl-4-(methyl(1-methyl-1H-imidazo[4,5-c]pyridin-6-yl)amino)benzamido) azetidine-1-carboxylate. The reaction mixture was stirred at room temperature for 2 h then concentrated in vacuo. The residue was dissolved in DCM and stirred with MP-carbonate for 10 mins The mixture was filtered, concentrated in vacuo and used in the next step without further purification.

¹H NMR δ (ppm)(CHCl₃-d): 8.72 (1H, s, ArH), 7.85 (1H, s, ArH), 7.69 (1H, d, ArH), 7.63 (1H, s, ArH), 7.26 (1H, d, ArH), 6.76 (1H, d, NH), 5.92 (1H, s, ArH), 5.00 (1H, m, CH), 4.07 (2H, m, CH), 3.64-3.56 (2H, m, CH), 3.53 (3H, s, CH3), 3.49 (3H, s, CH3), 2.56 (2H, q, CH2), 1.80 (1H, s, NH), 1.19 (3H, t, CH3).

LCMS (15 cm_Bicarb_GeminiNX_HPLC_MeCN) Rt 7.85 (min) m/z 365 (MH+).

### Step 2: 3-Ethyl-4-(methyl(1-methyl-1H-imidazo[4,5-c]pyridin-6-yl)amino)-N-(1-(methylsulfonyl)azetidin-3-yl)benzamide

Methanesulphonyl chloride (15 μL, 0.187 mmol) was added to a solution of N-(azetidin-3-yl)-3-ethyl-4-(methyl(1-methyl-1H-imidazo[4,5-c]pyridin-6-yl)amino)benzamide (62 mg, 0.17 mmol) and triethylamine (47 μL, 0.34 mmol) in DCM (3 mL). The reaction mixture was stirred at room temperature for 4 h and concentrated in vacuo. The residue was purified by preparative HPLC to give the desired compound.

¹H NMR δ (ppm)(CHCl₃-d): 8.69 (1H, s, ArH), 7.83 (1H, s, ArH), 7.71-7.66 (2H, m, ArH), 7.27 (1H, d, ArH), 6.95 (1H, d, NH), 5.96 (1H, s, ArH), 4.97-4.86 (1H, m, CH), 4.23 (2H, m, CH), 4.09 (2H, dd, CH), 3.59 (3H, s, CH3), 3.45 (3H, s, CH3), 2.95 (3H, s, CH3), 2.55 (2H, q, CH2), 1.17 (3H, t, CH3).

LCMS (15 cm_Bicarb_GeminiNX_HPLC_MeCN) Rt 8.35 (min) m/z 443 (MH⁺).

### Compound 176: N-(2-Ethyl-4-(1-(methylsulfonyl)-1,2,3,6-tetrahydropyridin-4-yl)phenyl)-N,1-dimethyl-1H-imidazo[4,5-c]pyridin-6-amine

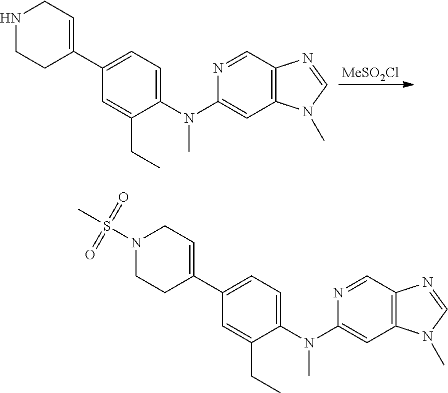

Methanesulfonyl chloride (26 μL, 0.46 mmol) was added to a solution of N-(2-ethyl-4-(1,2,3,6-tetrahydropyridin-4-yl)phenyl)-N,1-dimethyl-1H-imidazo[4,5-c]pyridin-6-amine (78 mg, 0.22 mmol) in DCM (1.5 mL) at 0° C. and stirred for 10 min. Triethylamine (47 μL, 0.46 mmol) was added and the reaction was stirred at room temperature for 1 d. The reaction mixture was concentrated in vacuo and the resulting residue was purified by preparative HPLC to give the desired compound.

¹H NMR δ (ppm)(CHCl₃-d): 8.72 (1H, d, ArH), 7.62 (1H, s, ArH), 7.39 (1H, d, ArH), 7.36-7.25 (1H, m, ArH), 7.19 (1H, d, ArH), 6.16-6.13 (1H, m, CH), 5.92 (1H, d, ArH), 4.01 (2H, m, CH), 3.59-3.52 (5H, m, CH3, CH), 3.44 (3H, s, CH3), 2.88 (3H, s, CH3), 2.75-2.71 (2H, m, CH), 2.54 (2H, q, CH2), 1.17 (3H, t, CH3).

LCMS (15 cm_Bicarb_GeminiNX_HPLC_MeCN) Rt 9.22 (min) m/z 426 (MH+).

### Compound 177: N-(2-Ethyl-4-(5-methyl-1,3,4-oxadiazol-2-yl)phenyl)-N,1-dimethyl-1H-imidazo[4,5-d]pyridin-6-amine

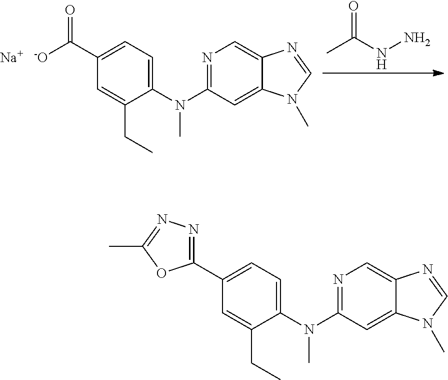

Phosphorous(V) oxychloride (6 mL) was added to a mixture of sodium 3-ethyl-4-(methyl(1-methyl-1H-imidazo[4,5-c]pyridin-6-yl)amino)benzoate (0.099 g, 0.3 mmol) and acetohydrazide (0.11 g, 1.5 mmol) and the resulting mixture was stirred at 100° C. for 7 h. The excess phosphorous(V) oxychloride was removed in vacuo and the residue was stirred with EtOAc and water. The layers were separated, dried (MgSO₄), filtered and concentrated in vacuo. The residue was purified by preparative HPLC to give the desired compound.

¹H NMR δ (ppm)(DMSO-d₆): 8.49 (1H, d, ArH), 8.03 (1H, s, ArH), 7.96 (1H, d, ArH), 7.89 (1H, dd, ArH), 7.40 (1H, d, ArH), 6.39 (1H, d, ArH), 3.65 (3H, s, CH3), 3.39 (3H, s, CH3), 2.61 (3H, s, CH3), 2.56-2.48 (2H, m, CH2), 1.14 (3H, t, CH3).

LCMS (10 cm_ESCI_Bicarb_MeCN) Rt 2.72 (min) m/z 349 (MH⁺).

### Compound 178: N-(2-Ethyl-4-(3-methyl-1,2,4-oxadiazol-5-yl)phenyl)-N,1-dimethyl-1H-imidazo[4,5-d]pyridin-6-amine

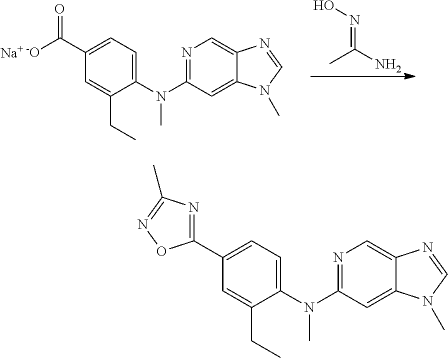

Sodium 3-ethyl-4-(methyl(1-methyl-1H-imidazo[4,5-c]pyridin-6-yl)amino)benzoate (0.099 g, 0.3 mmol) was stirred at room temperature with 1-(3-dimethylaminopropyl)-3-ethylcarbodiimide hydrochloride (0.0767 g, 0.4 mmol) and 2-hydroxypyridine-1-oxide (0.044 g, 0.4 mmol) in DMF (1.5 mL) for 1.5 h. N′-Hydroxyacetimidamide (0.0296 g, 0.4 mmol) was added and the mixture was heated to 60° C. for 7 h. The reaction mixture was concentrated in vacuo and the residue was dissolved in acetic acid (2.5 mL) and heated to 95° C. for 6 h. The reaction mixture was concentrated in vacuo and the residue purified by preparative HPLC to give the desired compound.

¹H NMR δ (ppm)(DMSO-d₆): 8.49 (1H, d, ArH), 8.08-7.98 (3H, m, ArH), 7.43 (1H, d, ArH), 6.47 (1H, d, ArH), 3.67 (3H, s, CH3), 3.39 (3H, s, CH3), 2.58 (2H, m, CH2), 2.44 (3H, s, CH3), 1.14 (3H, t, CH3).

LCMS (10 cm_ESCI_Bicarb_MeCN) Rt 3.00 (min) m/z 349 (MH⁺).

### Compound 179: N-Cyano-3-ethyl-4-(methyl(1-methyl-1H-imidazo[4,5-c]pyridin-6-yl)amino)benzamide

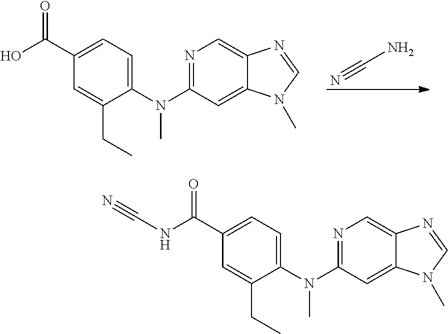

(2-(7-Aza-1H-benzotriazole-1-yl)-1,1,3,3-tetramethyluronium hexafluorophosphate) (0.19 g, 0.5 mmol) was added to a stirred suspension of 3-ethyl-4-(methyl(1-methyl-1H-imidazo[4,5-c]pyridin-6-yl)amino)benzoic acid (0.124 g, 0.4 mmol) and N-ethyl-N-isopropylpropan-2-amine (0.15 g, 1.2 mmol) in DMF (3 mL) at room temperature and the resulting solution was stirred at room temperature for 10 min. Cyanamide (0.025 g, 0.6 mmol) was added and stirring was continued for 20 h. The crude reaction mixture was purified by preparative HPLC to give the desired compound.

¹H NMR δ (ppm)(DMSO-d₆): 8.51 (1H, d, ArH), 8.19 (1H, s, ArH), 7.94 (1H, d, ArH), 7.85 (1H, dd, ArH), 7.33 (1H, d, ArH), 6.55 (1H, s, ArH), 3.70 (3H, s, CH3), 3.39 (3H, s, CH3), 2.54-2.44 (2H, m, CH), 1.12 (3H, t, CH3), (NH not observed).

LCMS (10 cm_ESCI_Bicarb_MeCN) Rt 2.20 (min) m/z 335 (MH⁺).

### Compound 181: N-(2-Ethyl-4-((2-methyl-1H-imidazol-1-yl)methyl)phenyl)-N,1-dimethyl-1H-imidazo[4,5-c]pyridin-6-amine

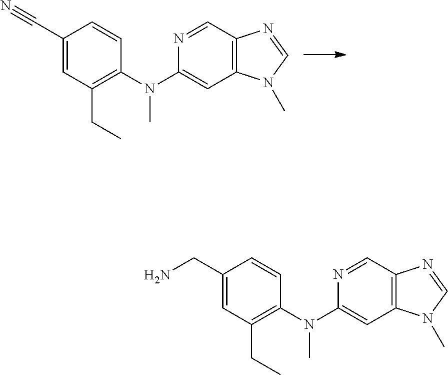 [SMILES: CCc1cc(C#N)ccc1N(C)c1cc2c(cn1)ncn2C ; CCc1cc(CN)ccc1N(C)c1cc2c(cn1)ncn2C]

3-Ethyl-4-(methyl(1-methyl-1H-imidazo[4,5-c]pyridin-6-yl)amino)benzonitrile (1 g, 3.43 mmol) was dissolved in 7 N NH₃ solution in MeOH (70 mL) to give a 0.05 M solution which was passed through an H-Cube (80° C., 80 Bar, flow rate: 1 mL min-1, H₂: controlled) fitted with a Raney Nickel. The solution was passed through the H-Cube once more under the same conditions to give complete conversion. The reaction mixture was concentrated in vacuo to give the desired compound.

¹H NMR δ (ppm)(CHCl₃-d): 8.72 (1H, s, ArH), 7.60 (1H, s, ArH), 7.34 (1H, s, ArH), 7.24 (1H, m, ArH), 7.17 (1H, d, ArH), 5.88 (1H, s, ArH), 3.94 (2H, s, CH2), 3.55 (3H, s, CH3), 3.44 (3H, s, CH3), 2.53 (2H, q, CH2), 1.62 (2H, s, NH₂), 1.16 (3H, t, CH3).

LCMS (15 cm_Bicarb_GeminiNX_HPLC_MeCN) Rt 8.07 (min) m/z 296 (MH⁺).

### Compound 182: N-(3-Ethyl-4-(methyl(1-methyl-1H-imidazo[4,5-c]pyridin-6-yl)amino)benzyl)acetamide

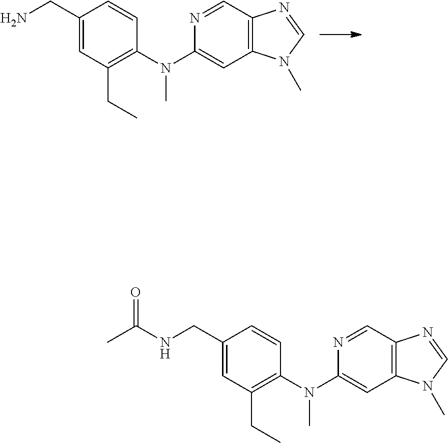 [SMILES: CCc1cc(CN)ccc1N(C)c1cc2c(cn1)ncn2C ; CCc1cc(CNC(C)=O)ccc1N(C)c1cc2c(cn1)ncn2C]

Acetyl chloride (16 μL, 0.22 mmol) was added to a solution of N-(2-ethyl-4-((2-methyl-1H-imidazol-1-yl)methyl)phenyl)-N,1-dimethyl-1H-imidazo[4,5-c]pyridin-6-amine (60 mg, 0.203 mmol) and triethylamine (85 μL, 0.61 mmol) in DCM (2 mL). The reaction mixture was stirred at room temperature for 1 d and concentrated in vacuo. The residue was purified by preparative HPLC to give the desired compound.

¹H NMR δ (ppm)(CHCl₃-d): 8.70 (1H, s, ArH), 7.61 (1H, s, ArH), 7.30-7.13 (3H, m, ArH), 5.91 (1H, s, ArH), 5.89 (1H, s, NH), 4.49 (2H, d, CH2), 3.58 (3H, s, CH3), 3.42 (3H, s, CH3), 2.52 (2H, q, CH2), 2.08 (3H, s, CH3), 1.15 (3H, t, CH3).

LCMS (15 cm_Bicarb_GeminiNX_HPLC_MeCN) Rt 8.18 (min) m/z 338 (MH+).

### Compound 184: N-(2-Ethyl-4-(2-morpholinopyrimidin-5-yl)phenyl)-N,1-dimethyl-1H-imidazo[4,5-d]pyridin-6-amine

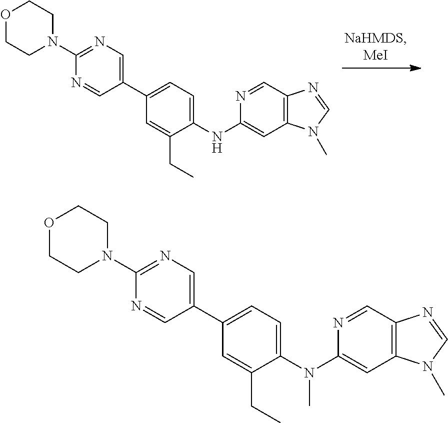 [SMILES: CCc1cc(-c2cnc(N3CCOCC3)nc2)ccc1Nc1cc2c(cn1)ncn2C ; CCc1cc(-c2cnc(N3CCOCC3)nc2)ccc1N(C)c1cc2c(cn1)ncn2C]

Sodium hexamethyldisilazide (1 M in THF, 0.40 mL, 0.40 mmol) was added dropwise to a solution of N-(2-ethyl-4-(2-morpholinopyrimidin-5-yl)phenyl)-1-methyl-1H-imidazo[4,5-c]pyridin-6-amine (150 mg, 0.36 mmol) in dry DMF (3 mL) at 0° C. After 10 min at 0° C. iodomethane (25 μL, 0.40 mmol) was added and the reaction mixture was allowed to warm to room temperature over 30 min. The reaction mixture was concentrated in vacuo and purified by preparative HPLC to give the desired compound.

¹H NMR δ (ppm)(DMSO-d₆): 8.80 (2H, s, ArH), 8.49 (1H, d, ArH), 8.03-7.96 (1H, m, ArH), 7.66 (1H, d, ArH), 7.57 (1H, dd, ArH), 7.25 (1H, d, ArH), 6.26 (1H, s, ArH), 3.79-3.75 (4H, m, CH), 3.72-3.68 (4H, m, CH), 3.62 (3H, s, CH3), 3.36 (3H, s, CH3), 2.55-2.44 (2H, m, CH2), 1.20-1.09 (3H, m, CH3).

LCMS (15 cm_Bicarb_GeminiNX_HPLC_MeCN) Rt 9.59 (min) m/z 430 (MH+).

### Compound 185: 3-Ethyl-4-(methyl(1-methyl-1H-imidazo[4,5-c]pyridin-6-yl)amino)benzoic acid

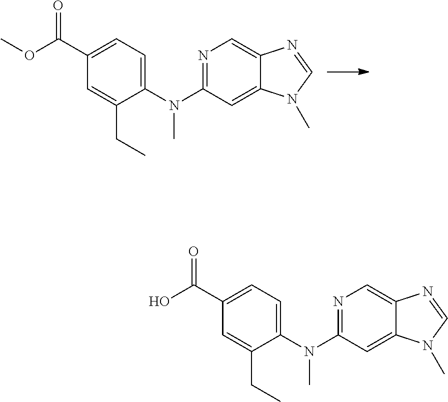 [SMILES: CCc1cc(C(=O)OC)ccc1N(C)c1cc2c(cn1)ncn2C ; CCc1cc(C(=O)O)ccc1N(C)c1cc2c(cn1)ncn2C]

Methyl 3-ethyl-4-(methyl(1-methyl-1H-imidazo[4,5-c]pyridin-6-yl)amino)benzoate (1.95 g, 6 mmol) was refluxed in MeOH (15 mL) with sodium hydroxide (0.24 g, 6 mmol) for 24 h. The mixture was concentrated in vacuo and the residue was treated with water (15 mL) to give a cloudy solution which was washed with EtOAc (10 mL) and neutralised (pH 7) with 2 M HCl aqueous solution to give an off-white powder which was filtered, washed with water and dried in vacuo to give the desired compound.

¹H NMR δ (ppm)(DMSO-d₆): 12.90 (1H, s, OH), 8.49 (1H, d, ArH), 8.02 (1H, s, ArH), 7.95 (1H, d, ArH), 7.86 (1H, dd, ArH), 7.30 (1H, d, ArH), 6.33 (1H, s, ArH), 3.64 (3H, s, CH3), 3.42-3.28 (3H, m, CH3), 2.53-2.44 (2H, m, CH2), 1.10 (3H, t, CH3).

LCMS (15 cm_Bicarb_GeminiNX_HPLC_MeCN) Rt 7.20 (min) m/z 311 (MH+).

### Compound 186: N-(2-Ethyl-4-(tetrahydro-2H-pyran-4-yl)phenyl)-N,1-dimethyl-1H-imidazo[4,5-c]pyridin-6-amine

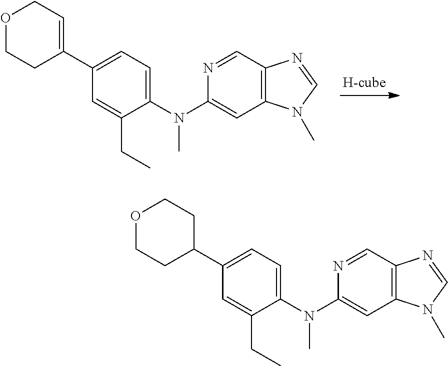

A solution of N-(4-(3,6-dihydro-2H-pyran-4-yl)-2-ethylphenyl)-N,1-dimethyl-1H-imidazo[4,5-c]pyridin-6-amine (150 mg, 0.43 mmol) in MeOH (9 mL) was passed through an H-Cube (60° C., 60 Bar, flow rate: 1 mL min-1, H₂: controlled) fitted with a 10 wt. % palladium on carbon twice. The resultant solution was concentrated in vacuo and purified by preparative HPLC to give the desired compound.

¹H NMR δ (ppm)(CHCl₃-d): 8.72 (1H, s, ArH), 7.60 (1H, s, ArH), 7.18-7.12 (3H, m, ArH), 5.88 (1H, d, ArH), 4.15-4.09 (2H, m, CH), 3.63-3.48 (5H, m, CH3, CH), 3.44 (3H, s, CH3), 2.85-2.76 (1H, m, CH), 2.52 (2H, q, CH2), 1.90-1.81 (4H, m, CH), 1.16 (3H, t, CH3).

LCMS (15 cm_Bicarb_GeminiNX_HPLC_MeCN) Rt 9.49 (min) m/z 351 (MH+).

### Compound 187: N-(2-Ethyl-4-(piperidin-4-yl)phenyl)-N,1-dimethyl-1H-imidazo[4,5-c]pyridin-6-amine

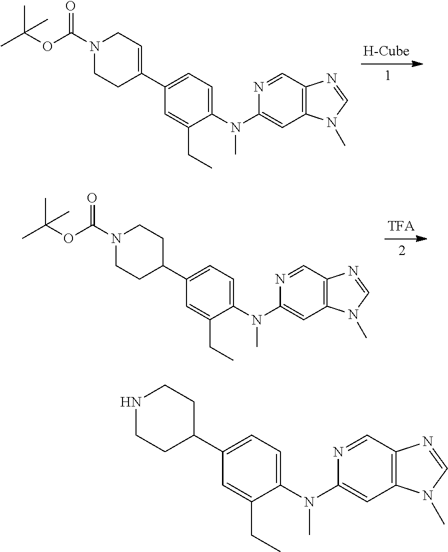 [SMILES: CCc1cc(C2=CCN(C(=O)OC(C)(C)C)CC2)ccc1N(C)c1cc2c(cn1)ncn2C ; CCc1cc(C2CCN(C(=O)OC(C)(C)C)CC2)ccc1N(C)c1cc2c(cn1)ncn2C ; CCc1cc(C2CCNCC2)ccc1N(C)c1cc2c(cn1)ncn2C]

### Step 1: tert-Butyl 4-(3-ethyl-4-(methyl(1-methyl-1H-imidazo[4,5-c]pyridin-6-yl)amino)phenyl)piperidine-1-carboxylate

A solution of tert-butyl 4-(3-ethyl-4-(methyl(1-methyl-1H-imidazo[4,5-c]pyridin-6-yl)amino)phenyl)-5,6-dihydropyridine-1(2H)-carboxylate (0.5 g, 1.12 mmol) in MeOH (25 mL) was passed through an H-Cube (60° C., 60 Bar, flow rate: 1 mL min-1, H₂: controlled) fitted with a 10 wt. % palladium on carbon. The resultant solution was concentrated in vacuo to give the desired compound.

¹H NMR δ (ppm)(CHCl₃-d): 8.72 (1H, d, ArH), 7.60 (1H, s, ArH), 7.20 (1H, s, ArH), 7.14-7.11 (2H, m, ArH), 5.87 (1H, d, ArH), 4.28 (2H, s, CH), 3.56 (3H, s, CH3), 3.43 (3H, s, CH3), 2.69 (2H, m, CH), 2.64 (1H, m, CH), 2.51 (2H, q, CH2), 1.90 (2H, d, CH), 1.67 (2H, s, CH), 1.50 (9H, s, CH3), 1.15 (3H, t, CH3).

LCMS (10 cm_ESCI_Bicarb_MeCN) Rt 3.77 (min) m/z 450 (MH+).

### Step 2: N-(2-Ethyl-4-(piperidin-4-yl)phenyl)-N,1-dimethyl-1H-imidazo[4,5-c]pyridin-6-amine

tert-Butyl 4-(3-ethyl-4-(methyl(1-methyl-1H-imidazo[4,5-c]pyridin-6-yl)amino)phenyl)piperidine-1-carboxylate (0.4 g, 0.89 mmol) was dissolved in DCM (5 mL). Trifluoroacetic acid (0.5 mL) was added and the reaction mixture was stirred at room temperature for 2 h. The reaction mixture was concentrated in vacuo. The resulting residue was dissolved in DCM and allowed to load under gravity onto a 10 g SCX column, washed with DCM and MeOH and eluted with 7 N NH₃ solution in MeOH:MeOH (1:5). The eluent was concentrated in vacuo. Of the material obtained, 100 mg was purified by preparative HPLC to give the desired compound.

¹H NMR δ (ppm)(CHCl₃-d): 8.72 (1H, d, ArH), 8.65 (1H, s, ArH), 7.62 (1H, s, ArH), 7.23 (1H, s, ArH), 7.16 (2H, d, ArH), 5.88 (1H, d, NH), 3.63-3.46 (5H, m, CH3, CH), 3.43 (3H, s, CH3), 3.03-2.92 (2H, m, CH), 2.85-2.73 (2H, m, CH), 2.66 (1H, s, CH), 2.57-2.47 (2H, m, CH2), 2.12-2.04 (2H, m, CH), 1.15 (3H, t, CH3).

LCMS (15 cm_Formic_ASCENTIS_HPLC_MeCN) Rt 7.04 (min) m/z 350 (MH+).

### Compound 190: N-(3-Ethyl-4-(methyl(1-methyl-1H-imidazo[4,5-c]pyridin-6-yl)amino)benzyl)methane sulfonamide

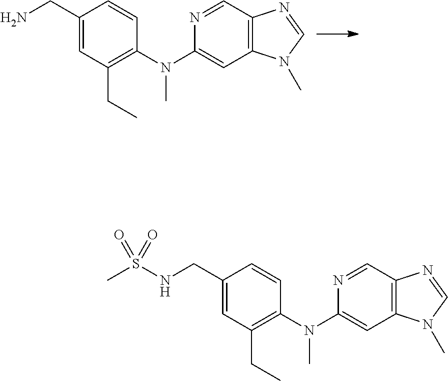 [SMILES: CCc1cc(CN)ccc1N(C)c1cc2c(cn1)ncn2C ; CCc1cc(CNS(C)(=O)=O)ccc1N(C)c1cc2c(cn1)ncn2C]

Methanesulphonyl chloride (17 μL, 0.22 mmol) was added to a solution of N-(2-ethyl-4-((2-methyl-1H-imidazol-1-yl)methyl)phenyl)-N,1-dimethyl-1H-imidazo[4,5-c]pyridin-6-amine (60 mg, 0.203 mmol) and triethylamine (85 μL, 0.61 mmol) in DCM (2 mL). The reaction mixture was stirred at room temperature for 1 d and concentrated in vacuo. The residue was purified by preparative HPLC to give the desired compound.

¹H NMR δ (ppm)(CHCl₃-d): 8.70 (1H, s, ArH), 7.62 (1H, s, ArH), 7.37 (1H, s, ArH), 7.31 (2H, m, ArH), 5.90 (1H, s, ArH), 4.69 (1H, t, NH), 4.38 (2H, d, CH2), 3.58 (3H, s, CH3), 3.43 (3H, s, CH3), 3.00 (3H, s, CH3), 2.53 (2H, q, CH2), 1.16 (3H, t, CH3).

LCMS (15 cm_Formic_ASCENTIS_HPLC_MeCN) Rt 7.93 (min) m/z 374 (MH+).

### Compound 191: Methyl 3-ethyl-4-(methyl(1-methyl-1H-imidazo[4,5-c]pyridin-6-yl)amino)benzyl carbamate

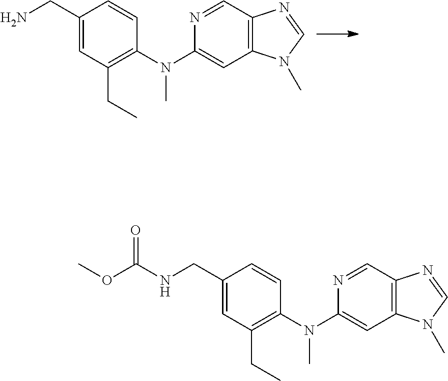 [SMILES: CCc1cc(CN)ccc1N(C)c1cc2c(cn1)ncn2C ; CCc1cc(CNC(=O)OC)ccc1N(C)c1cc2c(cn1)ncn2C]

Methyl chloroformate (17 μL, 0.22 mmol) was added to a solution of N-(2-ethyl-4-((2-methyl-1H-imidazol-1-yl)methyl)phenyl)-N,1-dimethyl-1H-imidazo[4,5-c]pyridin-6-amine (60 mg, 0.203 mmol) and triethylamine (85 μL, 0.61 mmol) in DCM (2 mL). The reaction mixture was stirred at room temperature for 1 d and concentrated in vacuo. The residue was purified by preparative HPLC to give the desired compound.

¹H NMR δ (ppm)(CHCl₃-d): 8.70 (1H, s, ArH), 7.61 (1H, s, ArH),7.28 (1H, s, ArH), 7.20 (2H, m, ArH), 5.89 (1H, s, ArH), 5.15 (1H, s, NH), 4.43 (2H, d, CH2), 3.74 (3H, s, CH3), 3.57 (3H, s, CH3), 3.42 (3H, s, CH3), 2.51 (2H, q, CH2), 1.15 (3H, t, CH3).

LCMS (15 cm_Bicarb_GeminiNX_HPLC_MeCN) Rt 8.73 (min) m/z 354 (MH+).

### Compound 192: 1-Ethyl-3-(3-ethyl-4-(methyl(1-methyl-1H-imidazo[4,5-c]pyridin-6-yl)amino)benzyl)urea

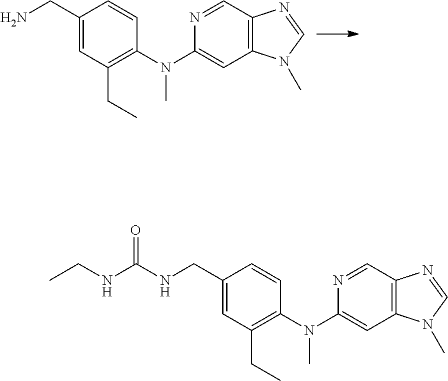 [SMILES: CCc1cc(CN)ccc1N(C)c1cc2c(cn1)ncn2C ; CCNC(=O)NCc1ccc(N(C)c2cc3c(cn2)ncn3C)c(CC)c1]

Ethyl isocyanate (18 μL, 0.22 mmol) was added to a solution of N-(2-ethyl-4-((2-methyl-1H-imidazol-1-yl)methyl)phenyl)-N,1-dimethyl-1H-imidazo[4,5-c]pyridin-6-amine (60 mg, 0.203 mmol) and triethylamine (85 μL, 0.61 mmol) in DCM (2 mL). The reaction mixture was stirred at room temperature for 1 d and concentrated in vacuo. The residue was purified by preparative HPLC to give the desired compound.

¹H NMR δ (ppm)(CHCl₃-d): 8.66 (1H, s, ArH), 7.61 (1H, s, ArH), 7.27 (1H, s, ArH), 7.21 (1H, d, ArH), 7.12 (1H, d, ArH), 5.94 (1H, s, ArH), 5.03 (1H, t, NH), 4.72 (1H, t, NH), 4.40 (2H, d, CH2), 3.58 (3H, s, CH3), 3.40 (3H, s, CH3), 3.25 (2H, m, CH2), 2.49 (2H, q, CH2), 1.17-1.08 (6H, m, CH3).

LCMS (15 cm_Bicarb_GeminiNX_HPLC_MeCN) Rt 8.34 (min) m/z 367 (MH+).

### Compound 193: 5-(3-Ethyl-4-(methyl(1-methyl-1H-imidazo[4,5-c]pyridin-6-yl)amino)phenyl)-1,3,4-thiadiazol-2-amine

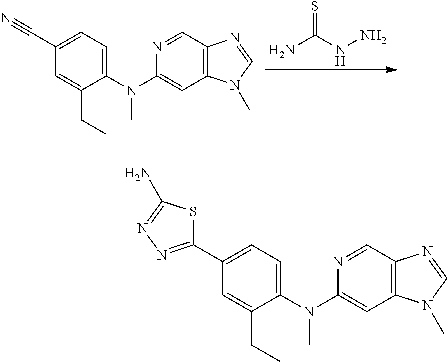

3-Ethyl-4-(methyl(1-methyl-1H-imidazo[4,5-c]pyridin-6-yl)amino)benzonitrile (100 mg, 0.34 mmol) and hydrazinecarbothioamide (49 mg, 0.54 mmol) were heated at reflux in trifluoroacetic acid (1.5 mL) for 1 d. The reaction mixture was cooled to room temperature and DCM and water were added. The pH was adjusted to pH 10 using 1M NaOH aq. solution. The aqueous was extracted with DCM, the combined organics were dried (hydrophobic frit) and concentrated in vacuo. The residue was purified by preparative HPLC to give the desired compound.

¹H NMR δ (ppm)(CHCl₃-d): 8.74 (1H, d, ArH), 7.88 (1H, d, ArH), 7.70 (1H, m, ArH), 7.63 (1H, s, ArH), 7.27 (1H, s, ArH), 5.96 (1H, d, ArH), 5.17 (2H, s, NH2), 3.58 (3H, s, CH3), 3.47 (3H, s, CH3), 2.58 (2H, q, CH2), 1.20 (3H, t, CH3).

LCMS (10 cm_ESCI_Bicarb_MeCN) Rt 2.44 (min) m/z 366 (MH+).

### Compound 195: 3-(4-(3-Ethyl-4-(methyl(1-methyl-1H-imidazo[4,5-c]pyridin-6-yl)amino)phenyl)-5,6-dihydropyridin-1(2H)-yl)-3-oxopropanenitrile

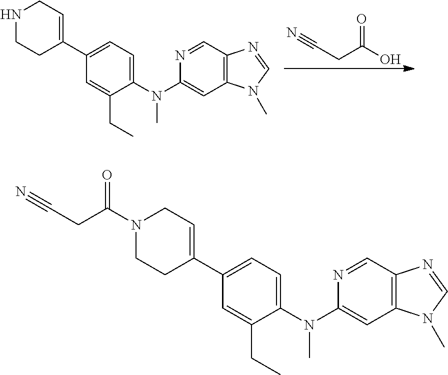

N-(2-Ethyl-4-(1,2,3,6-tetrahydropyridin-4-yl)phenyl)-N,1-dimethyl-1H-imidazo[4,5-c]pyridin-6-amine (110 mg, 0.32 mmol), 2-cyanoacetic acid (30 mg, 0.35 mmol), (2-(7-aza-1H-benzotriazole-1-yl)-1,1,3,3-tetramethyluronium hexafluorophosphate) (146 mg, 0.38 mmol) and diisopropylethylamine (0.22 mL, 1.28 mmol) were stirred in DMF (3 mL) at room temperature for 1 h. The crude reaction mixture was purified by preparative HPLC to give the desired compound.

### Alternative Synthesis for Compound 195: 3-(4-(3-Ethyl-4-(methyl(1-methyl-1H-imidazo[4,5-c]pyridin-6-yl)amino)phenyl)-5,6-dihydropyridin-1(2H)-yl)-3-oxopropanenitrile

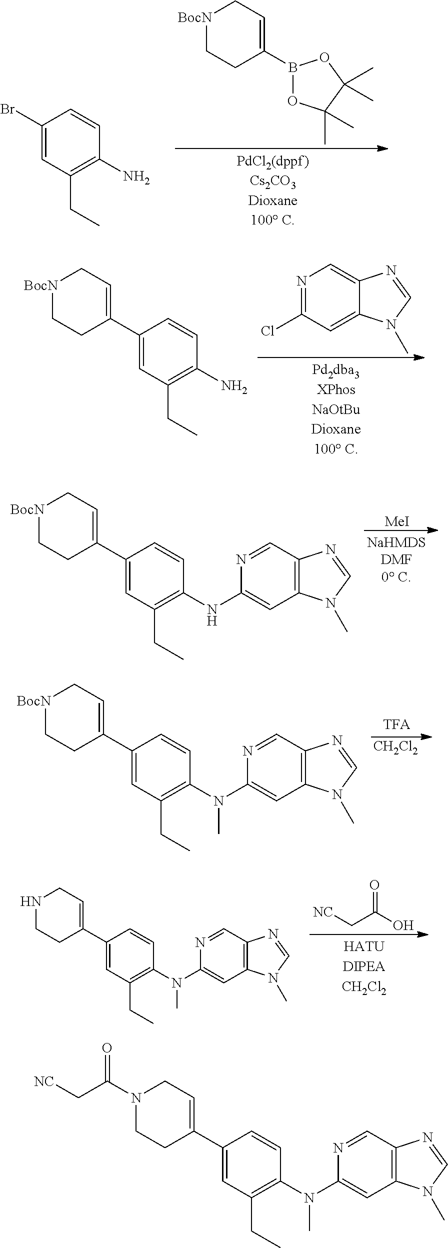

### Step 1: tert-butyl 4-(4-amino-3-ethylphenyl)-5,6-dihydropyridine-1(2H)-carboxylate

To a stirred mixture of 4-bromo-2-ethylaniline (13.5 g, 0.0675 mol), tert-butyl 4-(4,4,5,5-tetramethyl-1,3,2-dioxaborolan-2-yl)-5,6-dihydropyridine-1(2H)-carboxylate (25 g, 0.081 mmol), cesium carbonate (65 g, 0.2 mmol) in dioxane (350 mL) and water (60 mL) was added [1,1′-bis(diphenylphosphino)ferrocene]dichloropalladium(II) (2.5 g, 0.003 mol). The resulting mixture was flushed with N₂(g) and then heated to 100° C. for 18 h. After this time, the reaction mixture was cooled to room temperature and filtered through Hydroflo Super Cel® and then concentrated in vacuo. The residue was diluted with DCM (100 mL), filtered through Celite and then a hydrophobic frit. After concentrating in vacuo, purification by chromatography (eluent: 5% to 25% EtOAc in isohexane) gave the desired compound.

¹H NMR δ (ppm)(DMSO-d₆): 7.05-6.99 (2H, m), 6.59 (1H, d), 5.91 (1H, s), 4.92 (2H, s), 3.98 (2H, s), 3.53 (3H, s), 2.03 (1H, s), 1.46 (9H, t), 1.26-1.09 (5H, m).

### Step 2: tert-Butyl 4-(3-ethyl-4-((1-methyl-1H-imidazo[4,5-c]pyridin-6-yl)amino)phenyl)-5,6-dihydropyridine-1 (2H)-carboxylate

6-Chloro-1-methyl-1H-imidazo[4,5-c]pyridine (6.7 g, 0.04 mol), tert-butyl 4-(4-amino-3-ethylphenyl)-5,6-dihydropyridine-1(2H)-carboxylate (13.2 g, 0.044 mol), 2-dicyclohexylphosphino-2′,4′,6′-triisopropylbiphenyl (2.08 g, 0.004 mol), tris(dibenzylideneacetone)dipalladium(0) (2.0 g, 0.002 mol) and sodium tert-butoxide (6.4 g, 0.067 mol) were combined in degassed (N₂) dioxane (370 mL) and heated to 100° C. for 1.5 h. After this time the reaction mixture was cooled to room temperature, and filtered through Hydroflo Super Cel® and concentrated in vacuo. The resulting residue was purified by chromatography (eluent: 0 to 10% MeOH in DCM) to give the desired compound.

¹H NMR δ (ppm)(CHCl₃-d): 8.70 (1H, d), 7.70 (1H, s), 7.44 (1H, d), 7.33 (1H, d), 6.58 (1H, d), 6.31 (1H, s), 4.10 (2H, s), 2.73-2.61 (2H, m), 2.57 (2H, s), 1.67 (3H, s), 1.51 (9H, s), 1.29-1.21 (5H, m). 2H under solvent peak.

### Step 3: tert-butyl 4-(3-ethyl-4-(methyl(1-methyl-1H-imidazo[4,5-c]pyridin-6-yl)amino)phenyl)-5,6-dihydropyridine-1 (2H)-carboxylate

To a stirred solution of tert-butyl 4-(3-ethyl-4-((1-methyl-1H-imidazo[4,5-c]pyridin-6-yl)amino)phenyl)-5,6-dihydropyridine-1(2H)-carboxylate (8.02 g, 18.50 mmol) in DMF (80 mL) at 0° C., under a nitrogen atmosphere, was added sodium bis(trimethylsilyl)amide (1.0 M solution in THF, 20.3 mL, 20.35 mmol) dropwise. After stirring for 20 min, a solution of iodomethane (1.27 mL, 20.35 mmol) in DMF (5 mL) was added dropwise. After stirring at room temperature for 1.5 h, the reaction mixture was diluted with water and then concentrated in vacuo. The residue was diluted with water and DCM and separated using a hydrophobic frit and the organics were then concentrated in vacuo to give the desired compound which was used without further purification.

¹H NMR δ (ppm)(CHCl₃-d): 8.73-8.68 (1H, m), 7.60 (1H, s), 7.40 (1H, d), 7.32 (1H, dd), 7.19-7.08 (1H, m), 5.91-5.87 (1H, m), 4.11 (2H, s), 3.67 (2H, t), 3.73-3.34 (3H, m), 3.44 (3H, s), 2.57-2.48 (2H, m), 1.67 (2H, s), 1.50 (9H, s), 1.15 (3H, q). 1H under solvent peak.

### Step 4: N-(2-ethyl-4-(1,2,3,6-tetrahydropyridin-4-yl)phenyl)-N,1-dimethyl-1H-imidazo amine

To a stirred solution of tert-butyl 4-(3-ethyl-4-(methyl(1-methyl-1H-imidazo[4,5-c]pyridin-6-yl)amino)phenyl)-5,6-dihydropyridine-1(2H)-carboxylate (7.51 g, 16.78 mmol) in DCM (75 mL) was added trifluoroacetic acid (15 mL) and the resulting mixture was stirred for 8 h. After this time, the reaction mixture was concentrated in vacuo. The resulting residue was filtered through an SCX column, eluting with NH₃ (7M in MeOH): MeOH. Concentration in vacuo gave the desired compound which was used without further purification.

¹H NMR δ (ppm)(CHCl₃-d): 8.72 (1H, d), 7.60 (1H, s), 7.44-7.37 (1H, m), 7.33 (1H, dd), 7.16 (1H, d), 6.23-6.20 (1H, m), 5.90 (1H, d), 3.58 (2H, q), 3.58-3.50 (3H, m), 3.49 (1H, s), 3.51-3.36 (3H, m), 3.18-3.08 (2H, m), 2.58-2.48 (5H, m), 1.20-1.11 (3H, m). NH not observed.

### Step 5: 3-(4-(3-Ethyl-4-(methyl(1-methyl-1H-imidazo[4,5-c]pyridin-6-yl)amino)phenyl)-5,6-dihydropyridin-1(2H)-yl)-3-oxopropanenitrile

To a stirred solution of 2-cyanoacetic acid (1.45 g, 17.03 mmol) in DCM (110 mL) was added O-(7-azabenzotriazol-1-yl)-N,N,N′,N′-tetramethyluronium hexafluorophosphate (7.06 g, 18.58 mmol) and the mixture was stirred for 20 min. After this time N,N-diisopropylethylamine (10.8 mL, 61.92 mmol) followed by N-(2-ethyl-4-(1,2,3,6-tetrahydropyridin-4-yl)phenyl)-N,1-dimethyl-1H-imidazo[4,5-c]pyridin-6-amine (5.38 g, 15.48 mmol) were added and the resulting mixture was stirred for 8 h. After this time, the reaction mixture was diluted with water and the layers separated. The organics were washed with water, saturated sodium bicarbonate, dried (MgSO₄) and concentrated in vacuo. The resulting residue was purified by chromatography (eluent: 2% to 10% MeOH in EtOAc) to give the crude product. This was dissolved in DCM and washed with water (x9), dried (MgSO₄) and concentrated in vacuo to give the desired compound.

¹H NMR δ (ppm)(DMSO-d₆): 8.51 (1H, s, ArH), 8.14 (1H, s, ArH), 7.48 (1H, dd, ArH), 7.43-7.37 (1H, m, ArH), 7.20 (1H, d, ArH), 6.36 (1H, s, CH), 6.23 (1H, d, ArH), 4.16 (2H, s, CH), 4.10 (2H, s, CH), 3.74-3.51 (5H, m, CH, CH3), 3.36 (3H, s, CH3), 2.66-2.42 (4H, m, CH, CH2), 1.11 (3H, t, CH3).

LCMS (15 cm_Bicarb_GeminiNX_HPLC_CH3CN) t_{R} 8.89 (min) m/z 415 (MH⁺).

### Compound 207: N-(Cyanomethyl)-4-(3-ethyl-4-(methyl(1-methyl-1H-imidazo[4,5-c]pyridin-6-yl)amino)phenyl)-N-methyl-5,6-dihydropyridine-1(2H)-carboxamide

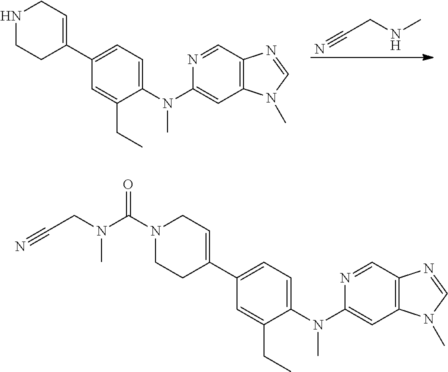

2-(Methylamino)acetonitrile (22 mg, 0.32 mmol) and triethylamine (45 μL, 0.32 mmol) were stirred in DCM (2 mL) at room temperature. Bis(trichloromethyl)carbonate (32 mg, 0.11 mmol) was added and the mixture stirred at room temperature for 1 h. Triethylamine (89 μL, 0.64 mmol) was added followed by a solution of N-(2-ethyl-4-(1,2,3,6-tetrahydropyridin-4-yl)phenyl)-N,1-dimethyl-1H-imidazo[4,5-c]pyridin-6-amine (111 mg, 0.32 mmol) in DCM (2 mL) and the reaction mixture was stirred at room temperature for 1 h. DCM and water were added and the mixture was filtered through a hydrophobic frit. The filtrate was concentrated in vacuo and purified by preparative HPLC to give the desired compound.

¹H NMR δ (ppm)(DMSO-d₆): 8.49 (1H, d, ArH), 7.98 (1H, s, ArH), 7.45 (1H, d, ArH), 7.38 (1H, dd, ArH), 7.16 (1H, d, ArH), 6.23 (1H, s, CH), 6.14 (1H, s, ArH), 4.14 (2H, s, CH2), 3.96 (2H, d, CH), 3.59 (3H, s, CH3), 3.49-3.42 (2H, m, CH2), 3.33 (3H, s, CH3), 2.90 (3H, s, CH3), 2.63-2.37 (4H, m, CH, CH2), 1.10 (3H, t, CH3).

LCMS (10 cm_ESCI_Bicarb_MeCN) Rt 2.94 (min) m/z 444 (MH+).

### Compound 211: N-(2-Ethyl-4-(2-(methylamino)pyrimidin-5-yl)phenyl)-N,1-dimethyl-1H-imidazo[4,5-d]pyridin-6-amine

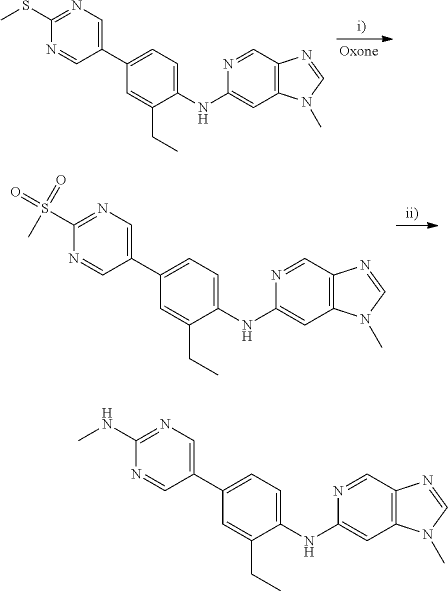 [SMILES: CCc1cc(-c2cnc(SC)nc2)ccc1Nc1cc2c(cn1)ncn2C ; CCc1cc(-c2cnc(S(C)(=O)=O)nc2)ccc1Nc1cc2c(cn1)ncn2C ; CCc1cc(-c2cnc(NC)nc2)ccc1Nc1cc2c(cn1)ncn2C]

### Step i: N-(2-Ethyl-4-(2-(methylsulfonyl)pyrimidin-5-yl)phenyl)-N,1-dimethyl-1H-imidazo[4,5-c]pyridin-6-amine

N-(2-Ethyl-4-(2-(methylthio)pyrimidin-5-yl)phenyl)-N,1-dimethyl-1H-imidazo[4,5-c]pyridin-6-amine (100 mg, 0.26 mmol) was stirred in THF (2 mL) at room temperature. A solution of Oxone (315 mg, 0.51 mmol) in water (4 mL) was added dropwise and the reaction mixture stirred at room temperature for 1 d. DCM and water were added and the organic washed with aq. NaHCO₃ solution, dried (hydrophobic frit) and concentrated in vacuo to give the desired compound.

### Step ii: N-(2-Ethyl-4-(2-(methylamino)pyrimidin-5-yl)phenyl)-N,1-dimethyl-1H-imidazo[4,5-c]pyridin-6-amine

N-(2-Ethyl-4-(2-(methylsulfonyl)pyrimidin-5-yl)phenyl)-N,1-dimethyl-1H-imidazo[4,5-c]pyridin-6-amine (100 mg, 0.24 mmol) and methylamine (2 M in THF, 1 mL, 2 mmol) were heated to 75° C. using microwave irradiation for 30 min. The reaction mixture was concentrated in vacuo and the residue was purified by preparative HPLC to give the desired compound.

¹H NMR δ (ppm)(CHCl₃-d): 8.74 (1H, d, ArH), 8.60 (2H, s, ArH), 7.62 (1H, s, ArH), 7.48 (1H, d, ArH), 7.41 (1H, m, ArH), 7.31 (1H, m, ArH), 5.97 (1H, d, ArH), 5.20 (1H, s, NH), 3.59 (3H, s, CH3), 3.47 (3H, s, CH3), 3.08 (3H, d, CH3), 2.59 (2H, q, CH2), 1.20 (3H, t, CH3).

LCMS (10 cm_ESCI_Bicarb_MeCN) Rt 2.70 (min) m/z 374 (MH+).

### Compound 212: N-(2-Ethyl-4-(2-(oxetan-3-yloxy)pyrimidin-5-yl)phenyl)-N,1-dimethyl-1H-imidazo[4,5-c]pyridin-6-amine

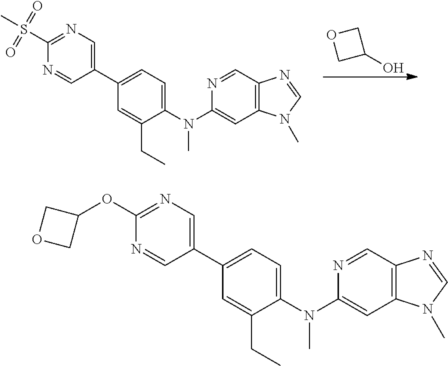

N-(2-Ethyl-4-(2-(methylsulfonyl)pyrimidin-5-yl)phenyl)-N,1-dimethyl-1H-imidazo[4,5-c]pyridin-6-amine (100 mg, 0.24 mmol), oxetan-3-ol (21 μL, 0.35 mmol) and potassium tert-butoxide (93 mg, 0.83 mmol) were heated at reflux in THF (4 mL) for 1 d. DCM and water were added and the layers separated with a hydrophobic frit. The filtrated was concentrated in vacuo and the residue was purified by preparative HPLC to give the desired compound.

¹H NMR δ (ppm)(CHCl₃-d): 8.78-8.70 (3H, m, ArH), 7.64 (1H, s, ArH), 7.52 (1H, d, ArH), 7.45 (1H, m, ArH), 7.33 (1H, d, ArH), 6.00 (1H, d, ArH), 5.72-5.66 (1H, m, CH), 5.03 (2H, m, CH), 4.85 (2H, m, CH), 3.61 (3H, s, CH3), 3.47 (3H, s, CH3), 2.60 (2H, q, CH2), 1.21 (3H, t, CH3).

LCMS (15 cm_Formic_ASCENTIS_HPLC_MeCN) Rt 7.22 (min) m/z 417 (MH+).

### Compound 213: N-Ethyl-4-(3-ethyl-4-(methyl(1-methyl-1H-imidazo[4,5-c]pyridin-6-yl)amino)phenyl)-5,6-dihydropyridine-1(2H)-carboxamide

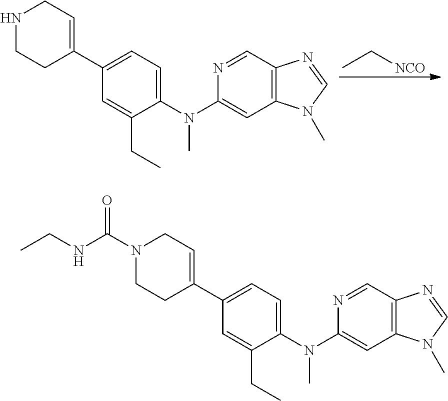

Ethyl isocyanate (27 μL, 0.34 mmol) was added to a solution of N-(2-ethyl-4-(1,2,3,6-tetrahydropyridin-4-yl)phenyl)-N,1-dimethyl-1H-imidazo[4,5-c]pyridin-6-amine (78 mg, 0.23 mmol) and triethylamine (27 μL, 0.34 mmol) in DCM (1.8 mL) at room temperature. The reaction mixture was stirred at room temperature for 2 h and concentrated in vacuo. The resulting residue was purified by preparative HPLC to give the desired compound.

¹H NMR δ (ppm)(CHCl₃-d): 8.72 (1H, d, ArH), 7.61 (1H, s, ArH), 7.40 (1H, d, ArH), 7.32 (1H, m, ArH), 7.19 (1H, s, ArH), 6.11 (1H, s, CH), 5.91 (1H, d, ArH), 4.42 (1H, s, NH), 4.06-4.03 (2H, m, CH), 3.67 (2H, m, CH), 3.56 (3H, s, CH3), 3.45 (3H, s, CH3), 3.38-3.30 (2H, m, CH), 2.63 (2H, d, CH), 2.54 (2H, q, CH2), 1.18 (6H, t, CH3).

### Compound 216: Cyanomethyl 4-(3-ethyl-4-(methyl(1-methyl-1H-imidazo[4,5-c]pyridin-6-yl)amino)phenyl)-5,6-dihydropyridine-1(2H)-carboxylate

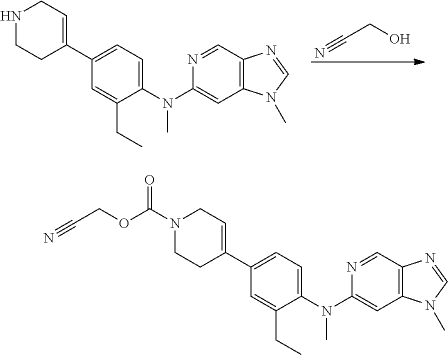

2-Hydroxyacetonitrile solution (70% in water, 30 μL, 0.37 mmol) and triethylamine (52 μL, 0.37 mmol) were stirred in DCM (2 mL) at room temperature. Bis(trichloromethyl)carbonate (37 mg, 0.12 mmol) was added and the mixture stirred at room temperature for 1 h. Triethylamine (0.1 mL, 0.74 mmol) was added followed by a solution of N-(2-ethyl-4-(1,2,3,6-tetrahydropyridin-4-yl)phenyl)-N,1-dimethyl-1H-imidazo[4,5-c]pyridin-6-amine (130 mg, 0.37 mmol) in DCM (2 mL) and the reaction mixture was stirred at room temperature for 4 d. DCM and water were added and the mixture was filtered through a hydrophobic frit. The filtrate was concentrated in vacuo and purified by preparative HPLC to give the desired compound.

¹H NMR δ (ppm)(DMSO-d₆): 8.49 (1H, d, ArH), 7.98 (1H, s, ArH), 7.45 (1H, d, ArH), 7.37 (1H, dd, ArH), 7.16 (1H, ArH), 6.22 (1H, d, CH), 6.15 (1H, s, ArH), 4.99 (2H, s, CH2), 4.12 (2H, s, CH), 3.71-3.56 (5H, m, CH, CH3), 3.34 (3H, s, CH3), 2.61-2.38 (4H, m, CH, CH2), 1.16-1.06 (3H, m, CH3).

LCMS (10 cm_ESCI_Bicarb_MeCN) Rt 3.12 (min) m/z 431 (MH+).

### Compound 222: 2-(4-(3-Ethyl-4-(methyl(1-methyl-1H-imidazo[4,5-c]pyridin-6-yl)amino)phenyl)-1H-pyrazol-1-yl)acetamide

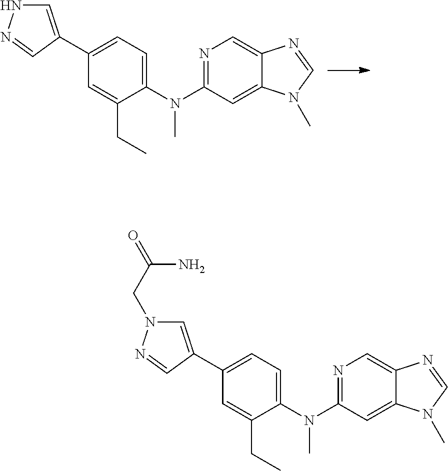 [SMILES: CCc1cc(-c2cn[nH]c2)ccc1N(C)c1cc2c(cn1)ncn2C ; CCc1cc(-c2cnn(CC(N)=O)c2)ccc1N(C)c1cc2c(cn1)ncn2C]

Sodium bis(trimethylsilyl)amide solution (1 M in THF, 0.33 mL, 0.33 mmol) was added to a solution of N-(2-ethyl-4-(1H-pyrazol-4-yl)phenyl)-N,1-dimethyl-1H-imidazo[4,5-c]pyridin-6-amine (99 mg, 0.3 mmol) in DMF (0.7 mL) and the resulting solution was stirred at room temperature for 15 min. 2-Bromoacetamide (41 mg, 0.3 mmol) was added and stirring was continued for 5 h. The resulting mixture was filtered and the filtrate was purified by preparative HPLC to give the desired compound.

¹H NMR δ (ppm)(CHCl₃-d): 8.75-8.72 (1H, m, ArH), 7.94 (1H, d, ArH), 7.78 (1H, d, ArH), 7.62 (1H, s, ArH), 7.53-7.49 (1H, m, ArH), 7.43 (1H, dd, ArH), 7.29-7.18 (1H, m, ArH), 6.26 (1H, s, NH), 5.94 (1H, d, ArH), 5.53 (1H, s, NH), 4.87 (2H, s, CH2), 3.57 (3H, s, CH3), 3.46 (3H, s, CH3), 2.56 (2H, q, CH2), 1.19 (3H, t, CH3).

LCMS (15 cm_Bicarb_GeminiNX_HPLC_MeCN) Rt 8.13 (min) m/z 390 (MH+).

### Compound 234: 4-(3-Ethyl-4-(methyl(1-methyl-1H-imidazo[4,5-c]pyridin-6-yl)amino)phenyl)-N-(1,3,4-thiadiazol-5-yl)-5,6-dihydropyridine-1(2H)-carboxamide

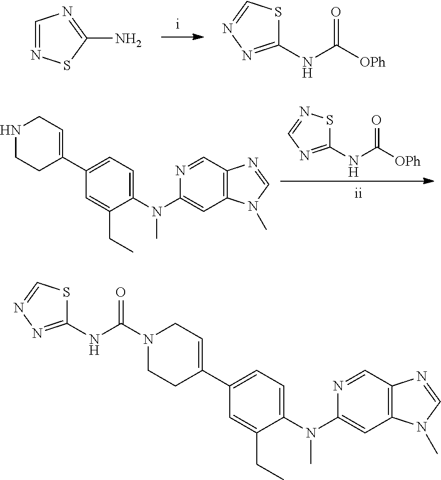

### Step i): [1,3,4]Thiadiazol-5-yl-carbamic acid phenyl ester

Phenyl chloroformate (1.26 g, 8.1 mmol) was added slowly to a mixture of 1,3,4-thiadiazol-5-amine (0.93 g, 8.1 mmol) and 4-dimethylaminopyridine (0.05 g, 0.41 mmol) in pyridine (10 mL) at 0° C. The reaction mixture was stirred at room temperature for 3 h and poured into iced water. The resulting precipitate was filtered, washed with water and dried in vacuo to give the desired compound.

### Step ii): 4-(3-Ethyl-4-(methyl(1-methyl-1H-imidazo[4,5-c]pyridin-6-yl)amino)phenyl)-N-(1,3,4-thiadiazol-5-yl)-5,6-dihydropyridine-1(2H)-carboxamide

A solution of N-(2-ethyl-4-(1,2,3,6-tetrahydropyridin-4-yl)phenyl)-N,1-dimethyl-1H-imidazo[4,5-c]pyridin-6-amine (compound 187) (0.1 g, 0.29 mmol), phenyl 1,2,4-thiadiazol-5-ylcarbamate (64 mg, 0.29 mmol) and diisopropylethylamine (51 mL, 0.29 mmol) in DMF (3 mL) was stirred at room temperature for 1 d. The crude reaction mixture was purified by preparative HPLC to give the desired compound.

¹H NMR δ (ppm)(CHCl₃-d): 9.5 (1H, s, NH), 8.72 (1H, d, ArH), 8.25 (1H, s, ArH), 7.64 (1H, s, ArH), 7.40 (1H, d, ArH), 7.32 (1H, dd, ArH), 7.20 (1H, d, ArH), 6.14 (1H, s, CH), 5.94 (1H, s, ArH), 4.28 (2H, s, CH), 3.89-3.83 (2H, m, CH), 3.58 (3H, s, CH3), 3.45 (3H, s, CH3), 2.73 (2H, s, CH), 2.55 (2H, q, CH2), 1.17 (3H, t, CH3).

LCMS (10 cm_ESCI_Formic_MeCN) t_{R} 2.59 (min) m/z 475 (MH⁺).

### Compound 252: tert-Butyl 3-ethyl-4-(methyl(1-methyl-1H-imidazo[4,5-c]pyridin-6-yl)amino)benzylcarbamate

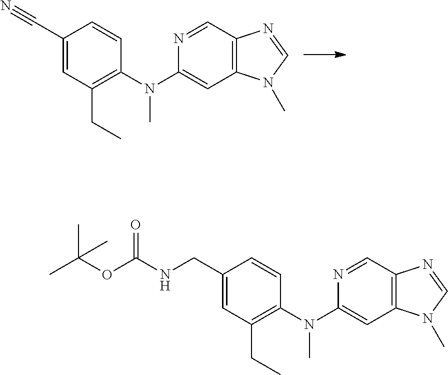 [SMILES: CCc1cc(C#N)ccc1N(C)c1cc2c(cn1)ncn2C ; CCc1cc(CNC(=O)OC(C)(C)C)ccc1N(C)c1cc2c(cn1)ncn2C]

3-Ethyl-4-(methyl(1-methyl-1H-imidazo[4,5-c]pyridin-6-yl)amino)benzonitrile (1 g, 3.4 mmol) was stirred in MeOH (20 mL) and cooled in a salt/ice bath. Then di-tert-butyl dicarbonate (1.48 g, 6.8 mmol) and NiCl₂.6H₂O (190 mg, 0.68 mmol) were added, followed by NaBH₄ (1.28 g, 34 mmol) which was added slowly over 20 mins. The reaction mixture was allowed to warm to room temperature and concentrated in vacuo. The residue was dissolved in EtOAc and the organics were washed with sat. aq. NaHCO₃ solution, brine, dried (MgSO₄), filtered and concentrated in vacuo. A small sample was purified by preparative HPLC to give the desired compound.

¹H NMR δ (ppm)(CHCl₃-d): 8.73 (1H, d, ArH), 7.63 (1H, s, ArH), 7.32-7.13 (3H, m, ArH), 5.90 (1H, s, ArH), 4.99 (1H, s, NH), 4.39 (2H, d, CH2), 3.58 (3H, s, CH3), 3.45 (3H, s, CH3), 2.53 (2H, q, CH2), 1.51 (9H, s, CH3), 1.16 (3H, t, CH3).

LCMS (10 cm_ESCI_Bicarb_MeCN) Rt 3.21 (min) m/z 396 (MH+).

### Compound 254: N-(3-Ethyl-4-(methyl(1-methyl-1H-imidazo[4,5-c]pyridin-6-yl)amino)benzyl)-2-methoxyacetamide

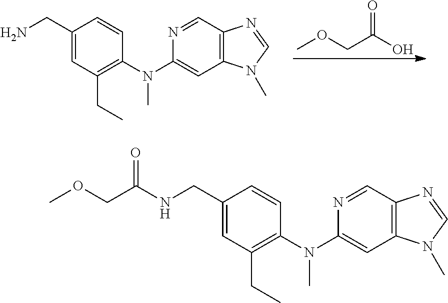

2-Methoxyacetic acid (48 mL, 0.56 mmol), 2-(7-aza-1H-benzotriazole-1-yl)-1,1,3,3-tetramethyluronium hexafluorophosphate (231 mg, 0.61 mmol) and diisopropylethylamine (0.22 mL, 1.28 mmol) were stirred in DMF (1 mL) at room temperature for 30 min. Then N-(4-(aminomethyl)-2-ethylphenyl)-N,1-dimethyl-1H-imidazo[4,5-c]pyridin-6-amine (150 mg, 0.51 mmol) in DMF (1.5 mL) was added and the reaction mixture stirred at room temperature for 8 h. The crude reaction mixture was purified by preparative HPLC to give the desired compound.

¹H NMR δ (ppm)(DMSO-d₆): 8.54 (1H, s, ArH), 8.48-8.36 (2H, m, NH, ArH), 7.35 (1H, s, ArH), 7.28-7.19 (2H, m, ArH), 6.67 (1H, s, ArH), 4.37 (2H, d, CH2), 3.90 (2H, s, CH2), 3.73 (3H, s, CH3), 3.37 (3H, s, CH3), 3.36 (3H, s, CH3), 2.45 (2H, q, CH2), 1.10 (3H, t, CH3).

LCMS (10 cm_ESCI_Bicarb_MeCN) Rt 2.46 (min) m/z 368 (MH+).

### Compound 257: 4-(3-Ethyl-4-(methyl(1-methyl-1H-imidazo[4,5-c]pyridin-6-yl)amino)phenyl)-N-(1,2,4-thiadiazol-5-yl)-5,6-dihydropyridine-1(2H)-carboxamide

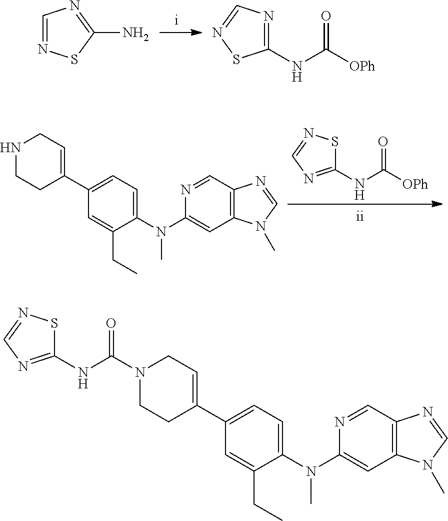

### Step i): [1,2,4]Thiadiazol-5-yl-carbamic acid phenyl ester

Phenyl chloroformate (1.26 g, 8.1 mmol) was added slowly to a mixture of 1,2,4-thiadiazol-5-amine (0.93 g, 8.1 mmol) and 4-dimethylaminopyridine (0.05 g, 0.41 mmol) in pyridine (10 mL) at 0° C. The reaction mixture was stirred at room temperature for 3 h and poured into ice water. The resulting precipitate was filtered, washed with water and dried in vacuo to give the desired compound.

### Step ii): 4-(3-ethyl-4-(methyl(1-methyl-1H-imidazo[4,5-c]pyridin-6-yl)amino)phenyl)-N-(1,2,4-thiadiazol-5-yl)-5,6-dihydropyridine-1(2H)-carboxamide

A solution of N-(2-ethyl-4-(1,2,3,6-tetrahydropyridin-4-yl)phenyl)-N,1-dimethyl-1H-imidazo[4,5-c]pyridin-6-amine (0.1 g, 0.29 mmol), phenyl 1,2,4-thiadiazol-5-ylcarbamate (64 mg, 0.29 mmol) and diisopropylethylamine (51 μL, 0.29 mmol) in DMF (3 mL) was stirred at room temperature for 1 d. The crude reaction mixture was purified by preparative HPLC to give the desired compound.

¹H NMR δ (ppm)(CHCl₃-d): 9.5 (1H, s, NH), 8.72 (1H, d, ArH), 8.25 (1H, s, ArH), 7.64 (1H, s, ArH), 7.40 (1H, d, ArH), 7.32 (1H, dd, ArH), 7.20 (1H, d, ArH), 6.14 (1H, s, CH), 5.94 (1H, s, ArH), 4.28 (2H, s, CH), 3.89-3.83 (2H, m, CH), 3.58 (3H, s, CH3), 3.45 (3H, s, CH3), 2.73 (2H, s, CH), 2.55 (2H, q, CH2), 1.17 (3H, t, CH3).

LCMS (10 cm_ESCI_Formic_MeCN) Rt 2.59 (min) m/z 475 (MH+).

### Compound 258: 1-{3-Ethyl-4-[methyl-(1-methyl-1H-imidazo[4,5-c]pyridin-6-yl)-amino]-benzyl}-3-cyano-urea

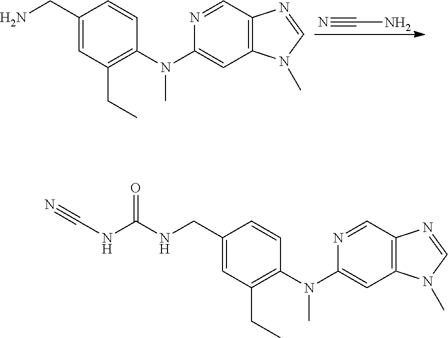

Cyanamide (23 mg, 0.55 mmol) and triethylamine (50 μL, 0.55 mmol) were stirred in DCM (2 mL) at room temperature. Bis(trichloromethyl)carbonate (50 mg, 0.17 mmol) was added and the mixture stirred at room temperature for 1 h. Triethylamine (0.1 mL, 1.0 mmol) was added followed by a solution of N-(4-(aminomethyl)-2-ethylphenyl)-N,1-dimethyl-1H-imidazo[4,5-c]pyridin-6-amine (166 mg, 0.56 mmol) in DCM (2 mL) and the reaction mixture was stirred at room temperature for 8 h. After this time, DCM and water were added and the mixture was filtered through a hydrophobic frit. The filtrate was concentrated in vacuo and purified by preparative HPLC to give the desired compound.

¹H NMR δ (ppm)(DMSO-d₆): 10.3 (1H, s, NH), 8.48 (1H, s, ArH), 7.98 (1H, s, ArH), 7.64 (1H, s, NH), 7.31-7.24 (1H, m, ArH), 7.23-7.11 (2H, m, ArH), 6.19-6.13 (1H, m, ArH), 4.28 (2H, d, CH2), 3.60 (3H, s, CH3), 3.33 (3H, s, CH3), 2.44 (2H, m, q, CH2), 1.09 (3H, t, CH3).

LCMS (25 cm_Acidic_Prodigy_HPLC) Rt 9.12 (min) m/z 364 (MH+).

### Compound 261: N-(3-ethyl-4-(methyl(1-methyl-1H-imidazo[4,5-c]pyridin-6-yl)amino)benzyl)-N-methylmethanesulfonamide

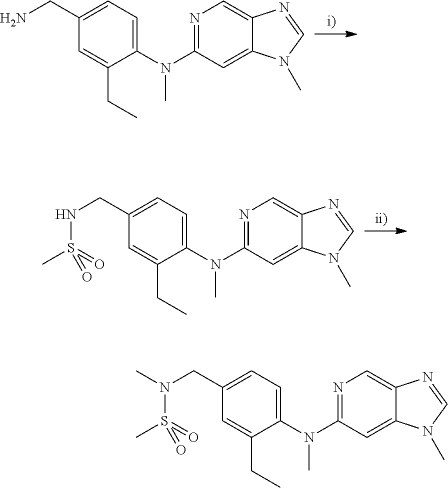 [SMILES: CCc1cc(CN)ccc1N(C)c1cc2c(cn1)ncn2C ; CCc1cc(CNS(C)(=O)=O)ccc1N(C)c1cc2c(cn1)ncn2C ; CCc1cc(CN(C)S(C)(=O)=O)ccc1N(C)c1cc2c(cn1)ncn2C]

### Step i): N-{3-Ethyl-4-[methyl-(1-methyl-1H-imidazo[4,5-c]pyridin-6-yl)-amino]-benzyl}-methanesulfonamide

Intermediate 8 (1 g) was dissolved in 20 mL DCM. MsCl (1.05 equiv, 271 μL) and Et₃N (2 equiv, 918 μL) were added and the reaction mixture was stirred overnight. Water was added and the two layer were separated. The organic layer was evaporated and the crude desired compound was used as such in the next reaction.

### Step ii): N-(3-ethyl-4-(methyl(1-methyl-1H-imidazo[4,5-c]pyridin-6-yl)amino)benzyl)-N-methylmethanesulfonamide

The product from the previous reaction was dissolved in THF (100 mL). NaH (2 equiv, 264 mg) was added in small portions. After 10 min, MeI (1.2 equiv, 250 μL) was added and the reaction mixture was stirred overnight at room temperature. LCMS showed full conversion. The mixture was evaporated and purified by column chromatography using EtOAc as a solvent, yielding the desired compound.

### Compound 262: [4-(1,1-Dioxoisothiazolidin-2-ylmethyl)-2-ethyl-phenyl]-methyl-(1-methyl-1H-imidazo[4,5-c]pyridin-6-yl)-amine

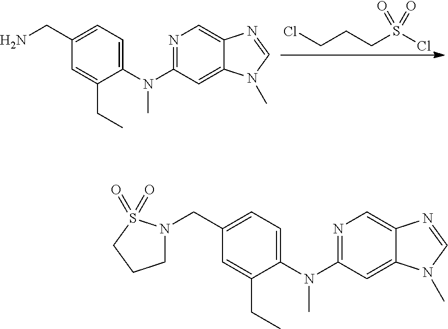

Intermediate 8 (1 g) was dissolved in THF (20 mL). Et₃N (1.5 equiv, 690 μL) and the sulfonyl chloride (1 equiv, 400 μL) were added and the mixture was stirred at room temperature. After 30 min, NaH (1.5 equiv, 200 mg) was added and the mixture was stirred for an additional 2 h. LCMS showed completion of the reaction. The mixture was evaporated and the crude was extracted using EtOAc and H₂O. The organic phase was evaporated and purified by chromatography using EtOAc/MeOH 9/1. The compound was further purified by preparative HPLC to yield the desired compound.

### Compound 264: N-(2-Methoxy-4-(thiophen-2-yl)phenyl)-1-methyl-1H-imidazo[4,5-c]pyridin-6-amine

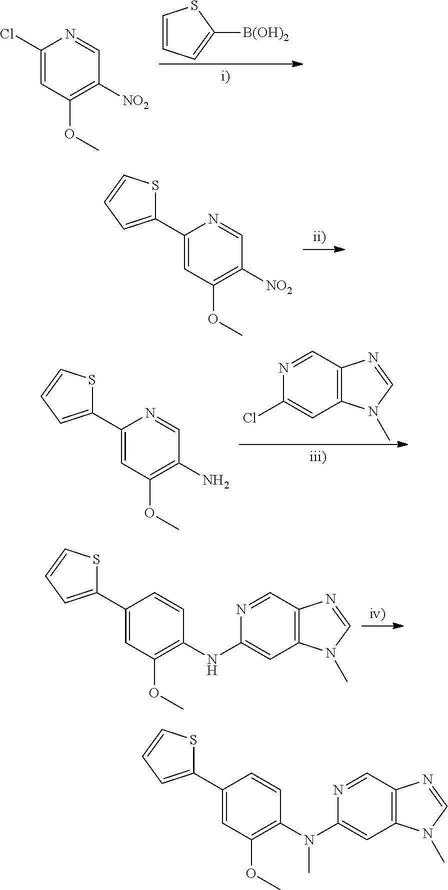

### Step i): 4-Methoxy-5-nitro-2-(thiophen-2-yl)pyridine

2-chloro-4-methoxy-5-nitropyridine (0.6 g, 3.19 mmol), thiophene-2-boronic acid (0.45 g, 3.5 mmol), [1,1′-bis(diphenylphosphino)ferrocene]dichloropalladium(II) (0.26 g, 0.32 mmol) and cesium carbonate (1.55 g, 4.8 mmol) in DMF (12 mL) were stirred and heated in a sealed tube at 90° C. for 18 h. The reaction mixture was cooled to room temperature, filtered through Celite and washed through with DCM. The filtrate was washed with water, dried (hydrophobic frit) and concentrated in vacuo. The residue was purified by column chromatography using silica gel and eluting with EtOAc in hexane (0-30%) to give the desired compound.

### Step ii): 4-Methoxy-6-(thiophen-2-yl)pyridin-3-amine

Iron powder (466 mg, 8.32 mmol) was added to a stirred suspension of ammonium chloride (133 mg, 2.49 mmol) and 4-methoxy-5-nitro-2-(thiophen-2-yl)pyridine (393 mg, 1.66 mmol) in a mixture of THF (3 mL), ethanol (3 mL) and water (1 mL). The resulting suspension was stirred at 90° C. for 1 h and cooled to room temperature. The mixture was filtered through Celite, washed through with DCM and the filtrate was washed with sat. aq. NaHCO₃ solution. The organics were dried (hydrophobic frit) and concentrated in vacuo to give the desired compound.

### Step iii): N-(2-Methoxy-4-(thiophen-2-yl)phenyl)-1-methyl-1H-imidazo[4,5-c]pyridin-6-amine

To stirred degassed (N₂) 1,4-dioxane (5 mL) was added 4-methoxy-6-(thiophen-2-yl)pyridin-3-amine (140 mg, 0.67 mmol), 6-chloro-1-methyl-1H-imidazo[4,5-c]pyridine (100 mg, 0.6 mmol), tris(dibenzylideneacetone)dipalladium(0) (28 mg, 0.03 mmol), 2-dicyclohexylphosphino-2′,4′,6′-triisopropylbiphenyl (29 mg, 0.09 mmol) and sodium tert-butoxide (86 mg, 0.9 mmol). The reaction mixture was heated to 100° C. for 1 d, cooled to room temperature, filtered through Celite and washed through with DSM. The reaction mixture was washed with water and the layers were separated, the aqueous layer was further extracted with DCM. The organics were combined, dried (hydrophobic filter) and concentrated in vacuo and the resulting residue was purified by column chromatography using silica and eluting with MeOH in EtOAc (0-10%) to give the desired compound.

### Step iv): N-(2-Methoxy-4-(thiophen-2-yl)phenyl)-1-methyl-1H-imidazo[4,5-c]pyridin-6-amine

Sodium hydride (60% in mineral oil, 10 mg, 0.25 mmol) was added to a solution of N-(2-methoxy-4-(thiophen-2-yl)phenyl)-1-methyl-1H-imidazo[4,5-c]pyridin-6-amine (64 mg, 0.19 mmol) in DMF (3 mL) at 0° C. and was allowed to warm to room temperature over 30 min. Iodomethane (17 μL, 0.28 mmol) was added and the solution stirred at room temperature for 45 min. Water and DCM was added and the aqueous extracted with DCM. The combined organics were dried (hydrophobic frit) and concentrated in vacuo. The residue was purified by preparative HPLC to give the desired compound.

¹H NMR δ (ppm) (CHCl₃-d): 8.75 (1H, s, ArH), 8.43 (1H, s, ArH), 7.67 (1H, s, ArH), 7.62 (1H, s, ArH), 7.45 (1H, d, ArH), 7.32 (1H, s, ArH), 7.18-7.14 (1H, m, ArH), 6.22 (1H, s, ArH), 3.93 (3H, s, CH3), 3.64 (3H, s, CH3), 3.48 (3H, s, CH3).

LCMS (10 cm_ESCI_Bicarb_MeCN) t_{R} 3.17 (min) m/z 352 (MH+).

### Compound 265: 3-(4-(3-Methoxy-4-(methyl(1-methyl-1H-imidazo[4,5-c]pyridin-6-yl)amino)phenyl)piperidin-1-yl)-3-oxopropanenitrile

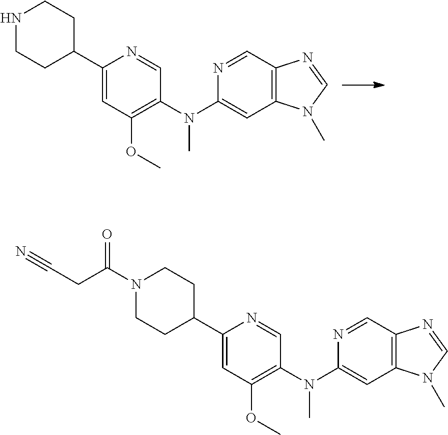 [SMILES: COc1cc(C2CCNCC2)ncc1N(C)c1cc2c(cn1)ncn2C ; COc1cc(C2CCN(C(=O)CC#N)CC2)ncc1N(C)c1cc2c(cn1)ncn2C]

To a stirred solution of N-(2-methoxy-4-(piperidin-4-yl)phenyl)-N,1-dimethyl-1H-imidazo[4,5-c]pyridin-6-amine (120 mg, 0.34 mmol) and (2-(7-aza-1H-benzotriazole-1-yl)-1,1,3,3-tetramethyluronium hexafluorophosphate) (155 mg, 0.40 mmol) in DMF (3 mL) was added diisopropylethylamine (0.24 mL, 1.36 mmol) followed by 2-cyanoacetic acid (32 mg, 0.37 mmol). The reaction mixture was stirred at room temperature for 2 h and then purified by preparative HPLC to give the desired compound.

LCMS (15 cm_Bicarb_GeminiNX_HPLC_CH3CN) t_{R} 7.99 (min) m/z 420 (MH⁺).

### Compound 268: tert-Butyl (1-(3-fluoro-4-(methyl(1-methyl-1H-imidazo[4,5-c]pyridin-6-yl)amino)phenyl)ethyl)carbamate

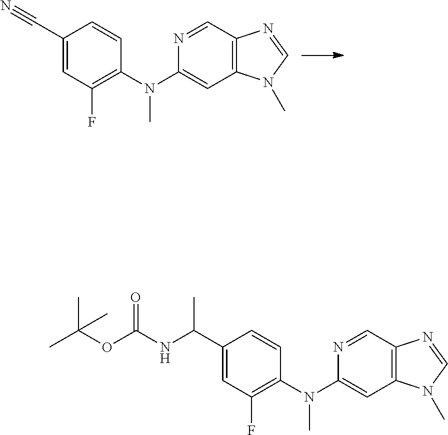 [SMILES: CN(c1cc2c(cn1)ncn2C)c1ccc(C#N)cc1F ; CC(NC(=O)OC(C)(C)C)c1ccc(N(C)c2cc3c(cn2)ncn3C)c(F)c1]

3-Fluoro-4-(methyl(1-methyl-1H-imidazo[4,5-c]pyridin-6-yl)amino)benzonitrile (200 mg, 0.71 mmol) and cerium chloride (175 mg, 0.71 mmol) were dissolved in tetrahydrofuran (20 mL) and stirred and cooled to 78° C. under N₂. Methyl lithium (1.6 M in Et₂O, 0.88 mL, 1.42 mmol) was added dropwise and the reaction mixture allowed to warm to room temperature. After 1 h di-tert-butyl dicarbonate (155 mg, 0.71 mmol) was added and the mixture stirred at room temperature for 1 d. The reaction mixture was concentrated in vacuo and EtOAc and water were added. The aqueous layer was extracted with EtOAc and the combined organics were dried (MgSO₄), filtered and concentrated in vacuo. The residue was purified by preparative HPLC to give the desired compound.

LCMS (15 cm_Bicarb_GeminiNX_HPLC_CH3CN) t_{R} 9.77 (min) m/z 400 (MH⁺).

### Compound 269: N-(3-Fluoro-4-(methyl(1-methyl-1H-imidazo[4,5-c]pyridin-6-yl)amino)benzyl)methanesulfonamide

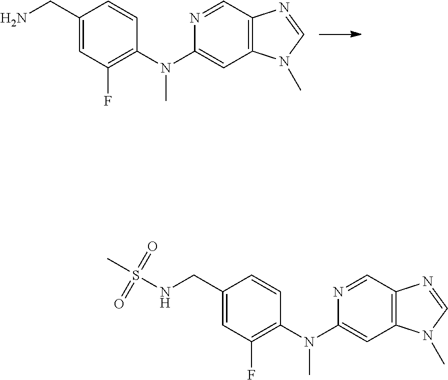 [SMILES: CN(c1cc2c(cn1)ncn2C)c1ccc(CN)cc1F ; CN(c1cc2c(cn1)ncn2C)c1ccc(CNS(C)(=O)=O)cc1F]

Triethylamine (0.44 mL, 3.14 mmol) was added to a solution of N-(4-(aminomethyl)-2-fluorophenyl)-N,1-dimethyl-1H-imidazo[4,5-c]pyridin-6-amine (0.32 g, 1.12 mmol) and methane sulfonylchloride (95 μL, 1.23 mmol) in DCM (10 mL) and the reaction mixture stirred at room temperature for 1 d. The reaction mixture was concentrated in vacuo and purified by preparative HPLC to give the desired compound.

¹H NMR δ (ppm)(DMSO-d₆): 8.44 (1H, s, ArH), 8.01 (1H, s, ArH), 7.60 (1H, s, ArH), 7.35 (1H, t, NH), 7.24-7.16 (2H, m, ArH), 6.55 (1H, s, ArH), 4.17 (2H, d, CH2), 3.65 (3H, s, CH3), 3.34 (3H, s, CH3), 2.90 (3H, s, CH3).

LCMS (15 cm_Bicarb_GeminiNX_HPLC_CH3CN) t_{R} 8.30 (min) m/z 364 (MH⁺).

### Compound 270:1-(3-ethyl-4-(methyl(1-methyl-1H-imidazo[4,5-c]pyridin-6-yl)amino)benzyl)-3-methylurea

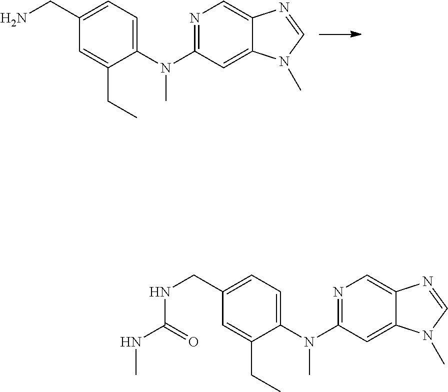 [SMILES: CCc1cc(CN)ccc1N(C)c1cc2c(cn1)ncn2C ; CCc1cc(CNC(=O)NC)ccc1N(C)c1cc2c(cn1)ncn2C]

Intermediate 8 (100 mg) was dissolved in THF (3 mL). Triphosgene (0.33 equiv, 33 mg) was added to the reaction under inert atmosphere. Et₃N (2 equiv, 92 μL) was added and the reaction mixture was stirred for 15 min at room temperature. A 2M solution of MeNH₂ in THF was added (1 equiv, 165 μL) and the mixture was stirred for 2 h at room temperature. LCMS showed presence of the desired end product. The reaction mixture was evaporated and the crude was extracted with DCM/H₂O. The organic layer was purified by preparative HPLC to yield the desired end compound.

### Compound 271 N-(2-ethyl-4-methoxyphenyl)-N,1-dimethyl-1H-imidazo[4,5-c]pyridin-6-amine; and compound 272 N-(4-ethoxy-2-ethylphenyl)-N,1-dimethyl-1H-imidazo[4,5-c]pyridin-6-amine

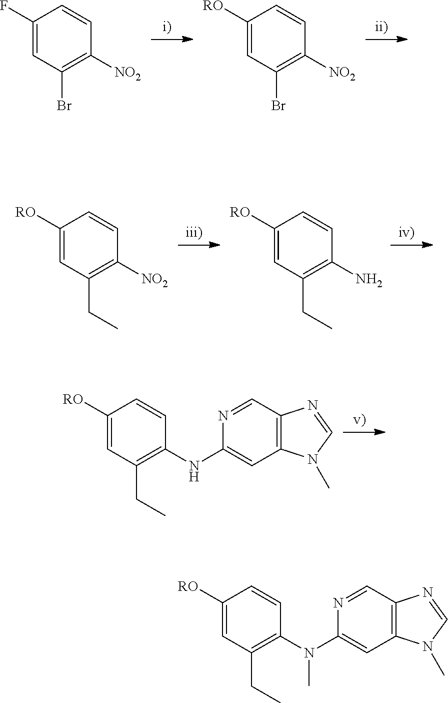

### Step i

(This procedure was used for both compound 271 and compound 272) 2-Bromo-4-fluoro-1-nitro-benzene (500 mg, 2.28 mmol, 1.0 equiv) was dissolved in a 1:1 mixture of DCM and NaOH aqueous solution (1N) (10 mL), alcohol (MeOH or EtOH) was added in large excess (2 mL). A catalytic amount of TBAB (3 mg) was added and the reaction was stirred at room temperature for 16 h. LCMS showed completion of the reaction. The mixture was partitioned between water and DCM. The aqueous layer was extracted with DCM (3×50 mL). The combined organic layer was washed with 0.1M HCl solution (4×30 mL), dried over Na₂SO₄, filtered and concentrated in vacuo to afford the desired material.

### Step ii)

2-Bromo-4-methoxy-1-nitro-benzene (582 mg, 2.52 mmol, 1.0 equiv), ethyl boronic acid (205 mg, 2.77 mmol, 1.1 equiv), Potassium carbonate (1.04 g, 7.56 mmol, 3.0 equiv) and PdCl₂(dppf) (103 mg, 0.13 mmol, 0.05 equiv) were dissolved in 1,4-dioxane/Water (4:1) (10 mL) and the mixture was stirred at 85° C. overnight. LCMS showed no traces of starting material. The reaction was cooled down to room temperature and concentrated in vacuo to remove volatiles. The residue was diluted in water and brine (50 mL) and the compound was extracted with DCM (3×50 mL). The combined organic layer was dried over Na₂SO₄, filtered and concentrated in vacuo to afford the crude material, which was used as such.

The same procedure was used for compound 272.

### Step iii)

2-ethyl-4-methoxy-1-nitro-benzene (489 mg, 1.0 equiv) was dissolved in acetic acid (40 mL) and stirred at 50° C. Zinc powder (1.75 g, 8.91 mmol, 10 equiv) was added and the mixture was heated to 80° C. After 1 h, LCMS showed full conversion. The reaction was cooled down, filtered and concentrated in vacuo. The residue was quenched with sat. NaHCO₃ solution and the compound was extracted with ethyl acetate (3×60 mL). The combined organic layer was dried over Na₂SO₄, filtered and concentrated in vacuo. The crude material was purified by column chromatography using the following condition: EtOAc/Petroleum ether (1:4) (10 CV), EtOAc/Petroleum ether (3:7) (10 CV), EtOAc/Petroleum ether (1:1) (10 CV), EtOAc (10 CV) which led to the desired product.

The same procedure was used for compound 272.

### Step iv)

2-Ethyl-4-methoxy-phenylamine (63 mg, 0.42 mmol, 1.0 equiv), 6-Chloro-1-methyl-1H-imidazo[4,5-c]-pyridine (77 mg, 0.46 mmol, 1.1 equiv), Cs₂CO₃ (411 mg, 1.26 mmol, 3.0 equiv), BINAP (26 mg, 0.04 mmol, 0.1 equiv) and Pd₂(dba)₃ (19 mg, 0.02 mmol, 0.05 equiv) were dissolved in dry dioxane (5 mL) under N₂ atmosphere. The mixture was sonicated for 5 nm under N₂ flow and then refluxed until full conversion was observed by LCMS. It was then cooled down and concentrated to dryness, diluted in DCM and washed with water (3×20 mL). The aqueous layer was extracted with DCM (3×30 mL) and the combined organic layer was dried over Na₂SO₄, filtered and concentrated to dryness. Purification was performed by column chromatography using the following eluant: EtOAc (20 CV), MeOH/EtOAc (1:19) (10 CV), MeOH/EtOAc (1:9) (10 CV) to afford the desired compound.

The same procedure was used for compound 272.

### Step v

(2-Ethyl-4-methoxy-phenyl)-(1-methyl-1H-imidazo[4,5-c]pyridin-6-yl)-amine (20 mg, 0.07 mmol, 1.0 equiv) was dissolved in dry THF (5 mL) under N₂ atmosphere, NaH (60% in mineral oil) (5 mg, 0.21 mmol, 3.0 equiv) was added and the reaction was stirred for 10 nm. MeI (8 μL, 0.08 mmol, 1.1 equiv) was added and the reaction mixture was stirred for 50 h under N₂. LCMS showed full conversion toward the expected material. The reaction was quenched with H2O (1 mL) and concentrated in vacuo to remove volatiles. The residue was diluted with DCM and washed with H₂O (3×20 mL). The organic layer was dried over Na₂SO₄, filtered and concentrated in vacuo. The crude material was submitted to preparatory chromatography for purification.

The same procedure was used for compound 272.

### Compound 273: N-(2-Methoxy-4-(piperidin-4-yl)phenyl)-N,1-dimethyl-1H-imidazo[4,5-c]pyridin-6-amine

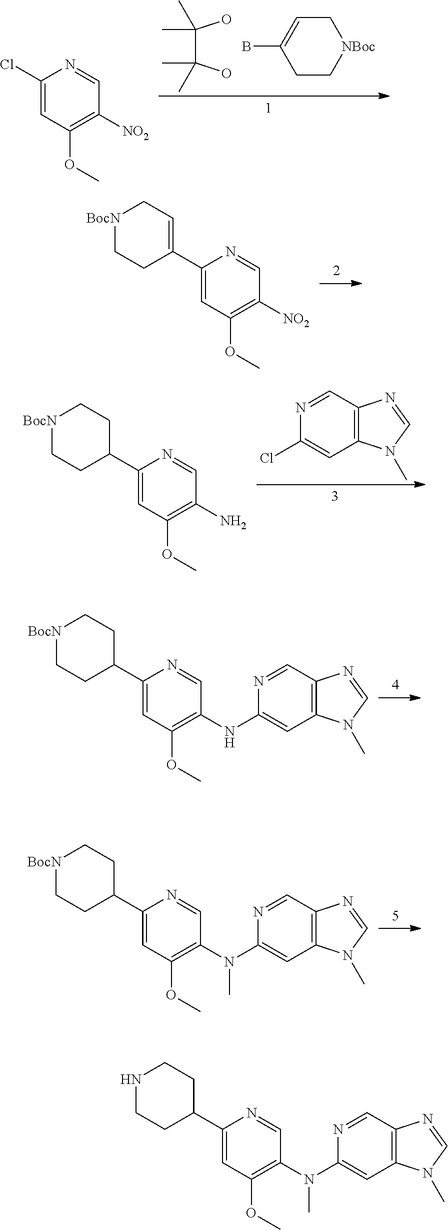

### Step 1: tert-Butyl 4-methoxy-5-nitro-5′,6′-dihydro-[2,4′-bipyridine]-1′(2′H)-carboxylate

As described for compound 264, step 1

### Step 2: tert-Butyl 4-(5-amino-4-methoxypyridin-2-yl)piperidine-1-carboxylate

tert-Butyl 4-methoxy-5-nitro-5′,6′-dihydro-[2,4′-bipyridine]-1′(2′H)-carboxylate (808 mg, 2.41 mmol) and 10% Pd/C (50 mg) in EtOH (20 mL) were subjected to H2(g) at atmospheric pressure. After 20 h the catalyst was filtered off and the filtrated concentrated in vacuo to give the desired compound.

### Step 3: tert-Butyl 4-(3-methoxy-4-((1-methyl-1H-imidazo[4,5-c]pyridin-6-yl)amino)phenyl)piperidine-1-carboxylate

As described for compound 264, step 3

### Step 4: tert-Butyl 4-(3-methoxy-4-(methyl(1-methyl-1H-imidazo[4,5-c]pyridin-6-yl)amino)phenyl)piperidine-1-carboxylate

As described for compound 264, step 4

LCMS (15 cm_Bicarb_GeminiNX_HPLC_CH3CN) tR 9.20 (min) m/z 453 (MH+).

### Step 5: N-(2-Methoxy-4-(piperidin-4-yl)phenyl)-N,1-dimethyl-1H-imidazo[4,5-c]pyridin-6-amine

tert-Butyl 4-(3-methoxy-4-(methyl(1-methyl-1H-imidazo[4,5-c]pyridin-6-yl)amino)phenyl)piperidine-1-carboxylate (440 mg, 0.97 mmol) was stirred in DCM (5 mL) and trifluoroacetic acid (2 mL) at room temperature for 4 h. The mixture was concentrated in vacuo and loaded onto an SCX column in DCM. MeOH (100 mL) was passed through the column and the compound was eluted with 7N NH3 in MeOH:MeOH (1:4). The filtrate was concentrated in vacuo to give the desired compound

### Compound 274: N-(2-(Difluoromethoxy)-4-(1-methyl-1H-pyrazol-4-yl)phenyl)-N,1-dimethyl-1H-imidazo[4,5-c]pyridin-6-amine

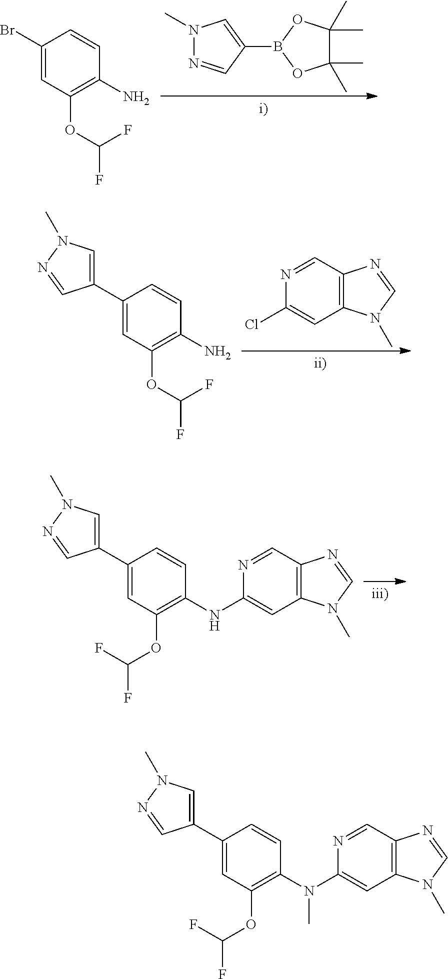

N-(2-(Difluoromethoxy)-4-(1-methyl-1H-pyrazol-4-yl)phenyl)-N,1-dimethyl-1H-imidazo[4,5-c]pyridin-6-amine was prepared using the conditions described in method G and B and starting with 4-bromo-2-(difluoromethoxy)aniline.

¹H NMR δ (ppm)(CHCl₃-d): 8.71 (1H, s, ArH), 7.79 (1H, s, ArH), 7.70 (1H, s, ArH), 7.66 (1H, s, ArH), 7.45-7.33 (3H, m, ArH), 6.44 (1H, t, CH), 6.33 (1H, s, ArH), 3.99 (3H, s, CH3), 3.70 (3H, s, CH3), 3.46 (3H, s, CH3).

LCMS (15 cm_Formic_ASCENTIS_HPLC_CH3CN) t_{R} 7.30 (min) m/z 385 (MH+).

### Compound 275: 6-(4-methoxybenzyl)-1-methyl-1H-imidazo[4,5-c]pyridine

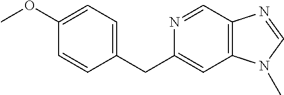 [SMILES: COc1ccc(Cc2cc3c(cn2)ncn3C)cc1]

(4-methoxybenzyl)zinc(II) chloride in solution in THF (0.45 mmol) was added to a solution of intermediate 1 (0.29 mmol) and Pd(PPh₃)₄ (0.09 mmol) in THF (1 mL). The reaction heated at 70° C. for 12 h. After completion, water and DCM were added and this mixture was filtered through a phase separator. The organic layers were concentrated under reduced pressure. The crude product was purified by preparative HPLC to give the desired compound.

Analytical: Waters Acquity UPLC BEH C18 1.7 μm, 2.1 mm ID×50 mm L (Part No. 186002350).

### Compound 286: 4-{3-Difluoromethoxy-4-[methyl-(1-methyl-1H-imidazo[4,5-c]pyridin-6-yl)-amino]-phenyl}-piperidine-1-carboxylic acid tert-butyl ester; and

### Compound 287: N-(2-(Difluoromethoxy)-4-(1-(methylsulfonyl)piperidin-4-yl)phenyl)-N,1-dimethyl-1H-imidazo[4,5-c]pyridin-6-amine

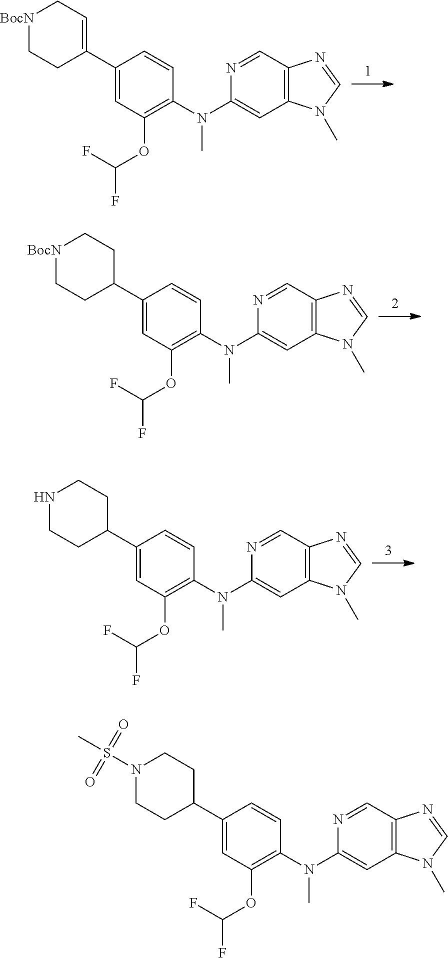

### Step 1: tert-Butyl 4-(3-(difluoromethoxy)-4-(methyl(1-methyl-1H-imidazo[4,5-c]pyridin-6-yl)amino)phenyl)piperidine-1-carboxylate

tert-Butyl 4-(3-(difluoromethoxy)-4-(methyl(1-methyl-1H-imidazo[4,5-c]pyridin-6-yl)amino)phenyl)-5,6-dihydropyridine-1(2H)-carboxylate (prepared using the conditions described in synthesis of intermediate 6 and starting with 4-bromo-2-(difluoromethoxy)aniline) (0.88 g, 1.81 mmol) and ammonium formate (1.14 g, 18.1 mmol) were dissolved in MeOH (50 mL). 10% Pd/C (0.2 g, 0.18 mmol) was added and the mixture stirred and heated at 80° C. for 1 d. The mixture was cooled to room temperature and filtered through Celite. Water and DCM were added to the filtrate and the aqueous was extracted with DCM, the combined organics were dried (MgSO₄), filtered and concentrated in vacuo to give the desired compound.

LCMS (10 cm_ESCI_Formic_MeCN) t_{R} 3.00 (min) m/z 388 (MH⁺).

### Step 2: N-(2-(Difluoromethoxy)-4-(piperidin-4-yl)phenyl)-N,1-dimethyl-1H-imidazo[4,5-c]pyridin-6-amine

tert-Butyl 4-(3-(difluoromethoxy)-4-(methyl(1-methyl-1H-imidazo[4,5-c]pyridin-6-yl)amino)phenyl)piperidine-1-carboxylate (0.42 g, 0.86 mmol) was stirred in DCM (4 mL) and trifluoroacetic acid (0.77 mL) at room temperature for 1 d. The reaction mixture was concentrated in vacuo and used in the next step without further purification.

### Step 3: N-(2-(Difluoromethoxy)-4-(1-(methylsulfonyl)piperidin-4-yl)phenyl)-N,1-dimethyl-1H-imidazo[4,5-c]pyridin-6-amine

N-(2-(Difluoromethoxy)-4-(piperidin-4-yl)phenyl)-N,1-dimethyl-1H-imidazo[4,5-c]pyridin-6-amine (115 mg, 0.30 mmol) and triethylamine (87 μL, 0.62 mmol) were stirred in DCM (5 mL) at 0° C. Methane sulfonylchloride (72 μL, 0.93 mmol) was added and the reaction mixture stirred at room temperature for 1 d. The reaction mixture was concentrated in vacuo and purified by preparative HPLC to give the desired compound.

¹H NMR δ (ppm)(DMSO-d₆): 8.41 (1H, d, ArH), 7.99 (1H, s, ArH), 7.27 (1H, d, ArH), 7.21-7.14 (2H, m, ArH), 6.99 (1H, t, CH), 6.50 (1H, d, ArH), 3.71-3.62 (5H, m, CH, CH3), 2.88 (3H, s, CH3), 2.74-2.63 (1H, m, CH), 2.84-2.76 (2H, m, CH), 2.47 (3H, s, CH3), 1.91 (2H, d, CH), 1.74-1.66 (2H, m, CH).

LCMS (15 cm_Bicarb_GeminiNX_HPLC_CH3CN) t_{R} 9.11 (min) m/z 466 (MH⁺).

### Compound 276: N-(2-Methoxy-4-(1-(methylsulfonyl)piperidin-4-yl)phenyl)-N,1-dimethyl-1H-imidazo[4,5-c]pyridin-6-amine

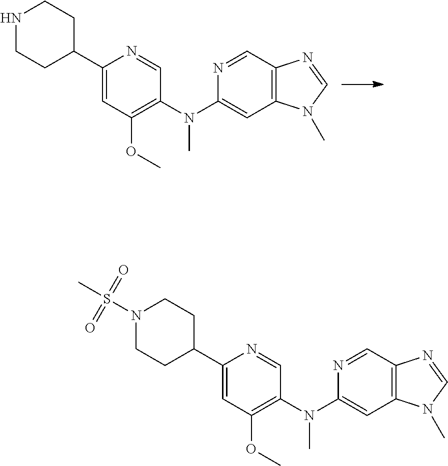 [SMILES: COc1cc(C2CCNCC2)ncc1N(C)c1cc2c(cn1)ncn2C ; COc1cc(C2CCN(S(C)(=O)=O)CC2)ncc1N(C)c1cc2c(cn1)ncn2C]

Methane sulfonylchloride (37 μL, 0.48 mmol) was added to a stirred solution of N-(2-methoxy-4-(piperidin-4-yl)phenyl)-N,1-dimethyl-1H-imidazo[4,5-c]pyridin-6-amine (140 mg, 0.40 mmol) and triethylamine (66 μL, 0.47 mmol) in DCM (3 mL) at 0° C. and allowed to warm to room temperature over 1 h. The reaction mixture was concentrated in vacuo and purified by preparative HPLC to give the desired compound.

¹H NMR δ (ppm)(DMSO-d₆): 8.43 (1H, s, ArH), 8.24 (1H, s, ArH), 8.01 (1H, s, ArH), 7.10 (1H, s, ArH), 6.43 (1H, s, ArH), 3.79 (3H, s, CH3), 3.76-3.64 (5H, m, CH2, CH3), 3.30 (3H, s, CH3), 2.94-2.79 (6H, m, CH, CH2, CH3), 2.03 (2H, d, CH2), 1.87-1.80 (2H, m, CH2).

LCMS (15 cm_Bicarb_GeminiNX_HPLC_CH3CN) t_{R} 8.19 (min) m/z 431 (MH+).

### Compound 277 [2-Fluoro-4-(1-methanesulfonyl-azetidin-3-yl)-phenyl]-methyl-(1-methyl-1H-imidazo[4,5-c]pyridin-6-yl)-amine; and

### Compound 278: 1-(3-{3-Fluoro-4-[methyl-(1-methyl-M-imidazo[4,5-c]pyridin-6-yl)-amino]-phenyl}-azetidin-1-yl)-ethanone

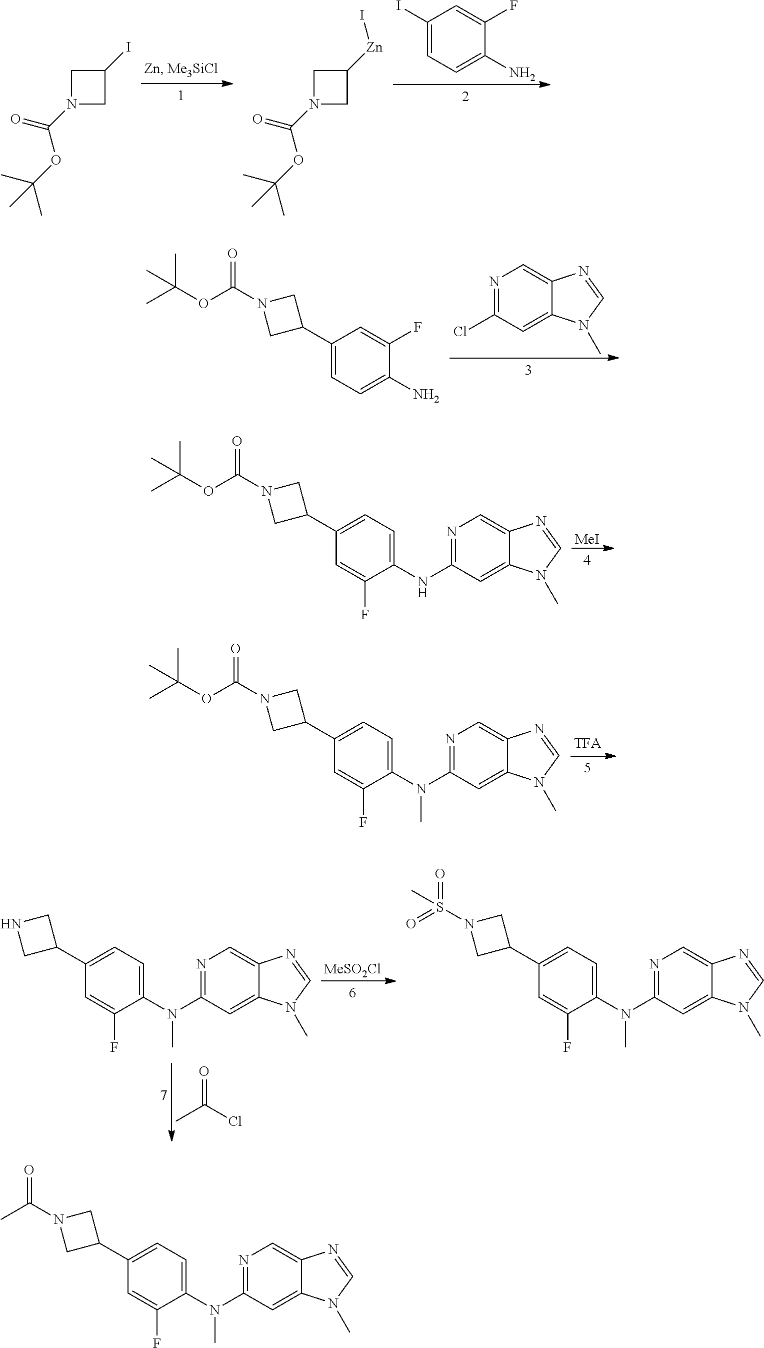

### Step 1: (1-(tert-Butoxycarbonyl)azetidin-3-yl)zinc(II) iodide

Zinc dust (<10 μM, 20.3 g) was stirred with 1M HCl (100 mL). After 2 h the suspension was filtered and the solid was washed with water (×2) then ethanol (×2) and finally with diethyl ether (×2). The solid was dried under vacuum and stored under nitrogen. Zinc dust (washed, 0.60 g, 9.16 mmol) was vigorously stirred in dimethylacetamide (4 mL) under nitrogen and the resulting suspension was heated to 65° C. Trimethylsilylchloride (0.12 g, 0.14 mL, 1.14 mmol) and 1,2-dibromoethane (0.098 mL, 1.14 mmol) were added and stirring was continued for 40 min. A solution of tert-butyl 3-iodoazetidine-1-carboxylate (2.0 g, 7.06 mmol) in dimethylacetamide (4 mL) was then added dropwise to the reaction mixture over 0.5 h. The resultant suspension was stirred at 65° C. for 0.5 h and allowed to cool to room temperature. The reaction mixture was used in the next step without work-up.

### Step 2: tert-Butyl 3-(4-amino-3-fluorophenyl)azetidine-1-carboxylate

2-Fluoro-4-iodoaniline (1.0 g, 4.24 mmol) in dimethylacetamide (8 mL) was added to the reaction mixture from Step 1 followed by [1,1′-bis(diphenylphosphino)ferrocene]dichloropalladium(II) (0.098 g, 0.12 mmol) and copper(I) iodide (0.05 g, 0.26 mmol). The reaction mixture was stirred and heated to 85° C. for 1.5 h then cooled to room temperature and quenched with sat. aq ammonium chloride solution. The mixture was extracted with EtOAc (×2) and the combined organics were dried (MgSO₄) and evaporated in vacuo to give the desired compound.

### Step 3: tert-Butyl 3-(3-fluoro-4-(1-methyl-1H-imidazo[4,5-c]pyridin-6-ylamino)phenyl)azetidine-1-carboxylate

6-Chloro-1-methyl-1H-imidazo[4,5-c]pyridine (0.38 g, 2.29 mmol), tert-butyl 3-(4-amino-3-fluorophenyl)azetidine-1-carboxylate (0.67 g, 2.51 mmol), cesium carbonate (2.23 g, 6.86 mmol), tris(dibenzylideneacetone)dipalladium(0) (0.052 g, 0.057 mmol) and 2,2′-bis(diphenylphosphino)-1,1′-binaphthyl (0.071, 0.11 mmol) were heated in 1,4-dioxane at 110° C. under nitrogen in a sealed tube for 20 h. The mixture was evaporated in vacuo and the residue was partitioned between DCM and water. The organic phase was separated and the aqueous phase extracted with DCM (×3), the combined organic phases were dried (MgSO₄) and evaporated in vacuo. The residue was purified by flash chromatography (5% EtOAc in isohexane to 100% EtOAc and then with 10% MeOH in DCM) to give the desired compound.

### Step 4: tert-butyl 3-(3-fluoro-4-(methyl(1-methyl-1H-imidazo[4,5-c]pyridin-6-yl)amino)phenyl)azetidine-1-carboxylate

Sodium hexamethyldisilazide (1.57 mL of a 1.0 M solution in THF, 1.57 mmol) was added dropwise to a stirred solution of tert-butyl 3-(3-fluoro-4-(1-methyl-1H-imidazo[4,5-c]pyridin-6-ylamino)phenyl)azetidine-1-carboxylate (0.57 g, 1.43 mmol) in DMF (10 mL) at 0° C. After 0.5 h at 0° C. iodomethane (0.097 g, 1.57 mmol) was added and the reaction mixture was allowed to warm to room temperature over 2 h. The reaction mixture was evaporated in vacuo and the residue was purified by flash chromatography (0 to 10% MeOH in DCM) to give the desired compound.

### Step 5: N-(4-(Azetidin-3-yl)-2-fluorophenyl)-N,1-dimethyl-1H-imidazo[4,5-c]pyridin-6-amine

To a stirred solution of tert-butyl 3-(3-fluoro-4-(methyl(1-methyl-1H-imidazo[4,5-c]pyridin-6-yl)amino)phenyl)azetidine-1-carboxylate (0.062 g, 1.43 mmol) in DCM was added trifluoroacetic acid (5 mL). The resultant solution was stirred at room temperature for 4 h before being evaporated in vacuo. The residue was loaded onto a 5 g SCX cartridge, washed with MeOH and eluted with 7N NH₃ solution in MeOH to give the desired compound.

### Step 6: N-(2-Fluoro-4-(1-(methylsulfonyl)azetidin-3-yl)phenyl)-N,1-dimethyl-1H-imidazo[4,5-c]pyridin-6-amine

Methanesulfonyl chloride (0.11 mL, 1.48 mmol) was added to a stirred solution of N-(4-(azetidin-3-yl)-2-fluorophenyl)-N,1-dimethyl-1H-imidazo[4,5-c]pyridin-6-amine (0.22 g, 0.71 mmol) and triethylamine (0.21 mL, 1.48 mmol) in DCM (5 mL) at 0° C. and allowed to warm to room temperature over 2 h. The reaction mixture was concentrated in vacuo and purified by preparative HPLC to give the desired compound.

¹H NMR δ (ppm)(DMSO-d₆): 8.43 (1H, d, ArH), 8.01 (1H, s, ArH), 7.38-7.26 (2H, m, ArH), 7.22 (1H, dd, ArH), 6.59 (1H, s, ArH), 4.21-4.13 (2H, m, CH), 3.99-3.84 (3H, m, CH), 3.66 (3H, s, CH3), 3.34 (3H, s, CH3), 3.06 (3H, s, CH3).

LCMS (10 cm_ESCI_Bicarb_MeCN) t_{R} 2.92 (min) m/z 390 (MH⁺).

Step 7: 1-(3-(3-Fluoro-4-(methyl(1-methyl-1H-imidazo[4,5-c]pyridin-6-yl)amino)phenyl)azetidin-1-yl)ethanone

Acetyl chloride (91 μL, 1.28 mmol) was added to a solution of N-(4-(azetidin-3-yl)-2-fluorophenyl)-N,1-dimethyl-1H-imidazo[4,5-c]pyridin-6-amine (0.20 g, 0.64 mmol) and triethylamine (0.18 mL, 1.28 mmol) in DCM (5 mL) at 0° C. and allowed to warm to room temperature over 2 h. The reaction mixture was concentrated in vacuo and purified by preparative HPLC to give the desired compound.

LCMS (15 cm_Formic_ASCENTIS_HPLC_CH3CN) t_{R} 6.89 (min) m/z 354 (MH⁺).

### Compound 279: 4-{3-Fluoro-4-[methyl-(1-methyl-M-imidazo[4,5-c]pyridin-6-yl)-amino]-phenyl}-piperidine-1-carboxylic acid tert-butyl ester

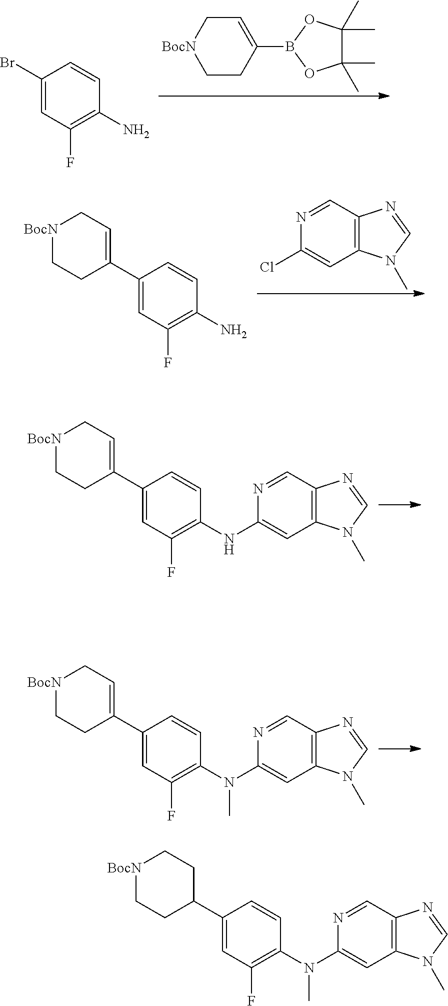

tert-Butyl 4-(3-fluoro-4-(methyl(1-methyl-1H-imidazo[4,5-c]pyridin-6-yl)amino)phenyl)piperidine-1-carboxylate was prepared using the conditions described for the preparation of Intermediate 6 starting from 4-bromo-2-fluoroaniline.

LCMS (10 cm_Formic_ACE 3 C18 AR_HPLC_CH3CN) t_{R} 9.67 (min) m/z 440 (MH⁺).

### Compound 280: N-(4-Ethyl-6-(1-methyl-1H-pyrazol-4-yl)pyridin-3-yl)-N,1-dimethyl-1H-imidazo[4,5-c]pyridin-6-amine

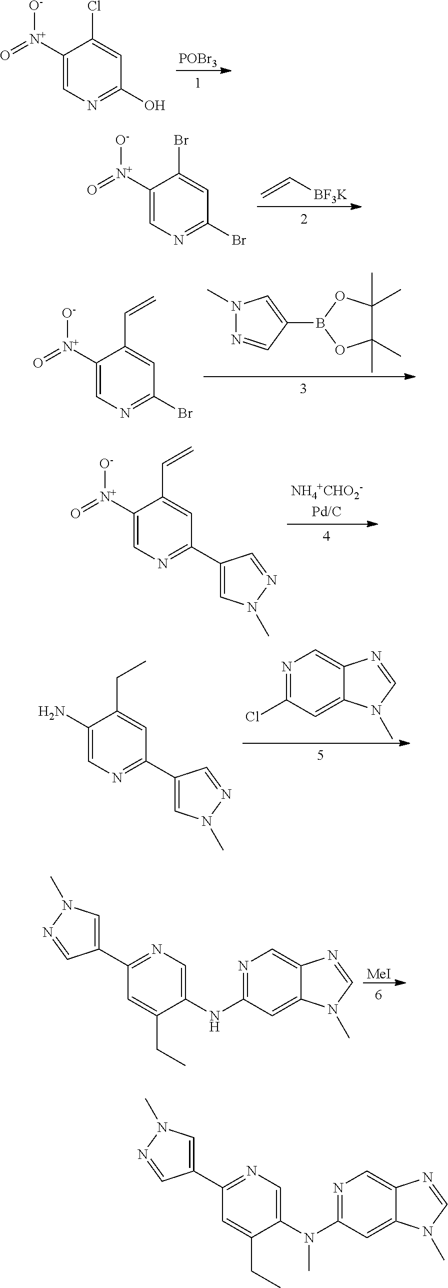

### Step 1: 2,4-Dibromo-5-nitropyridine

4-Chloro-5-nitropyridin-2-ol (3.06 g, 0.017 mol) and phosphorus(V) oxybromide (2.9 g, 0.01 mol) were stirred and refluxed in acetonitrile (70 mL) for 1 h, a further 1 g of phosphorus(V) oxybromide was added and reflux was continued for 1 h. The reaction mixture was poured on ice (400 mL) and extracted with EtOAc (3×100 mL). The layers were separated and the combined organics dried (hydrophobic frit) and concentrated in vacuo to give a brown solid which was recrystalised from isopropanol to give the desired compound.

### Step 2: 2-Bromo-5-nitro-4-vinylpyridine

A mixture of 2,4-dibromo-5-nitropyridine (1.48 g, 5.3 mmol), potassium trifluoro(vinyl)borate (0.6 g, 4.5 mmol) and sodium carbonate (1.68 g, 15.9 mmol) in dimethoxymethane (50 mL) and water (20 mL) was degassed (N₂). Tetrakis(triphenylphosphine)palladiun(0) (50 mg) was added and the mixture was stirred at 75° C. for 18 h. The reaction mixture was added to water (100 mL) and extracted with EtOAc (3×80 mL), the combined extracts were washed with saturated sodium chloride solution (80 mL), dried (MgSO₄), filtered and concentrated in vacuo. The residue was purified by flash chromatography (50% toluene in isohexane to toluene) to give the desired compound.

### Step 3: 2-(1-Methyl-1H-pyrazol-4-yl)-5-nitro-4-vinylpyridine

A stirred mixture of 2-bromo-5-nitro-4-vinylpyridine (250 mg, 1.09 mmol), 1-methyl-4-(4,4,5,5-tetramethyl-1,3,2-dioxaborolan-2-yl)-1H-pyrazole (210 mg, 1 mmol), sodium carbonate (2 mL of a 2 M solution in water) and dimethoxyethane (4 mL) was degassed (N₂). Tetrakis(triphenylphosphine)palladiun(0) (20 mg) was added and the mixture was heated using microwave irradiation at 100° C. for 10 min. The reaction mixture was added to water (30 mL) and extracted with EtOAc (3×20 mL), the combined extracts were dried (MgSO₄) and concentrated in vacuo to give an orange oil which was purified by flash chromatography (5% EtOAc in toluene to 100% EtOAc) to give the desired compound.

### Step 4: 4-Ethyl-6-(1-methyl-1H-pyrazol-4-yl)pyridin-3-amine

A stirred solution of 2-(1-methyl-1H-pyrazol-4-yl)-5-nitro-4-vinylpyridine (118 mg, 0.51 mmol) and ammonium formate (600 mg) in MeOH was degassed (N₂). 10% Pd/C (7 mg) was added and the mixture was refluxed for 1.5 h. The solvent was evaporated in vacuo and the residue was purified by flash chromatography (EtOAc to 10% 7N NH₃ in MeOH in EtOAc) to give the desired compound.

### Step 5: N-(4-Ethyl-6-(1-methyl-1H-pyrazol-4-yl)pyridin-3-yl)-1-methyl-1H-imidazo[4,5-c]pyridin-6-amine

A stirred mixture of 4-ethyl-6-(1-methyl-1H-pyrazol-4-yl)pyridin-3-amine (36 mg, 0.17 mmol), 6-chloro-1-methyl-1H-imidazo[4,5-c]pyridine (30 mg, 0.18 mmol), 2-dicyclohexylphosphino-2′,4′,6′-triisopropylbiphenyl (12 mg) and sodium tert-butoxide (30 mg, 0.3 mmol) in 1,4-dioxane (7 mL) was degassed (N₂). Tris(dibenzylideneacetone)dipalladium(0) (10 mg) was added and the mixture was refluxed for 3.5 h. The mixture was evaporated in vacuo and the residue was purified by flash chromatography (DCM to 20% 7N NH₃ in MeOH in DCM) to give the desired compound.

### Step 6: N-(4-Ethyl-6-(1-methyl-1H-pyrazol-4-yl)pyridin-3-yl)-N,1-dimethyl-1H-imidazo[4,5-c]pyridin-6-amine

A stirred solution of N-(4-ethyl-6-(1-methyl-1H-pyrazol-4-yl)pyridin-3-yl)-1-methyl-1H-imidazo[4,5-c]pyridin-6-amine (38 mg, 0.114 mmol) in dimethylformamide (3 mL) was treated with sodium hydride (60% in mineral oil, 7 mg, 0.17 mmol) and the mixture was stirred for 10 min. A solution of iodomethane (25 mg, 0.18 mmol) in dimethylformamide (1 mL) was added and stirring was continued for 2 h at room temperature. The mixture was treated with water (0.5 mL) and evaporated in vacuo. The residue was purified by flash chromatography (DCM to 20% 7N NH₃ in MeOH in DCM) and further purified by preparative HPLC to give the desired compound.

¹H NMR δ (ppm)(DMSO-d₆): 8.47 (1H, s, ArH), 8.29 (2H, d, ArH), 8.01 (2H, d, ArH), 7.65 (1H, s, ArH), 6.37 (1H, s, ArH), 3.90 (3H, s, CH3), 3.65 (3H, s, CH3), 3.38 (3H, s, CH3), 2.56-2.44 (2H, q, CH2), 1.14 (3H, t, CH3).

LCMS (10 cm_ESCI_Bicarb_MeCN) t_{R} 2.38 (min) m/z 348 (MH⁺).

### Compound 283: {3-Fluoro-4-[methyl-(1-methyl-1H-imidazo[4,5-c]pyridin-6-yl)-amino]-benzyl}-carbamic acid methyl ester

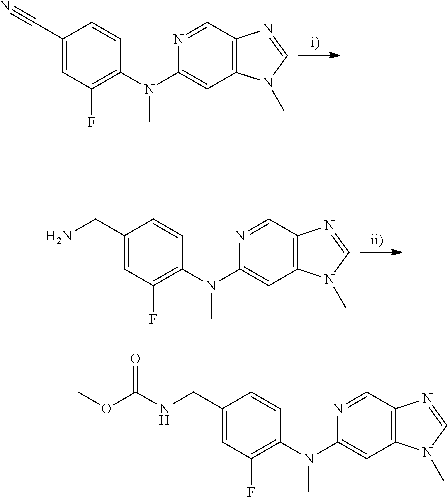

### Step i: {3-Fluoro-4-[methyl-(1-methyl-1H-imidazo[4,5-c]pyridin-6-yl)-amino]-benzyl}-amine

NiCl₂.6H₂O (0.85 g, 3.56 mmol) and trifluoroacetic acid (2.11 mL) was added to a solution of 3-fluoro-4-(methyl(1-methyl-1H-imidazo[4,5-c]pyridin-6-yl)amino)benzonitrile (1 g, 3.56 mmol) in MeOH (40 mL) at 0° C. NaBH₄ (1.08 g, 28.4 mmol) was added slowly and the mixture stirred at 0° C. for 2 h and room temperature for 1 d. The mixture was filtered through Celite and the filtrate concentrated in vacuo. EtOAc and NaOH solution (2 M, aq.) were added. The aqueous layer was extracted with EtOAc and the combined organics were dried (MgSO₄), filtered and concentrated in vacuo to give the desired compound which was used in the next step without further purification.

### Step ii: {3-Fluoro-4-[methyl-(1-methyl-1H-imidazo[4,5-c]pyridin-6-yl)-amino]-benzyl}-carbamic acid methyl ester

Triethylamine (0.44 mL, 3.14 mmol) was added to a solution of N-(4-(aminomethyl)-2-fluorophenyl)-N,1-dimethyl-1H-imidazo[4,5-c]pyridin-6-amine (0.32 g, 1.12 mmol) and methyl chloroformate (95 μL, 1.23 mmol) in DCM (10 mL) and the reaction mixture stirred at room temperature for 1 d. The reaction mixture was concentrated in vacuo and purified by preparative HPLC to give the desired compound.

¹H NMR δ (ppm)(DMSO-d₆): 8.43 (1H, d, ArH), 8.00 (1H, s, ArH), 7.75-7.69 (1H, m, ArH), 7.31 (1H, t, NH), 7.13-7.07 (2H, m, ArH), 6.55 (1H, s, ArH), 4.18 (2H, d, CH2), 3.65 (3H, s, CH3), 3.54 (3H, s, CH3), 2.47 (3H, s, CH3).

LCMS (10 cm_ESCI_Bicarb_MeCN) t_{R} 2.43 (min) m/z 344 (MH⁺).

### Compound 289: N-(4-Ethyl-6-(thiophen-2-yl)pyridin-3-A-N,1-dimethyl-1H-imidazo[4,5-c]pyridin-6-amine

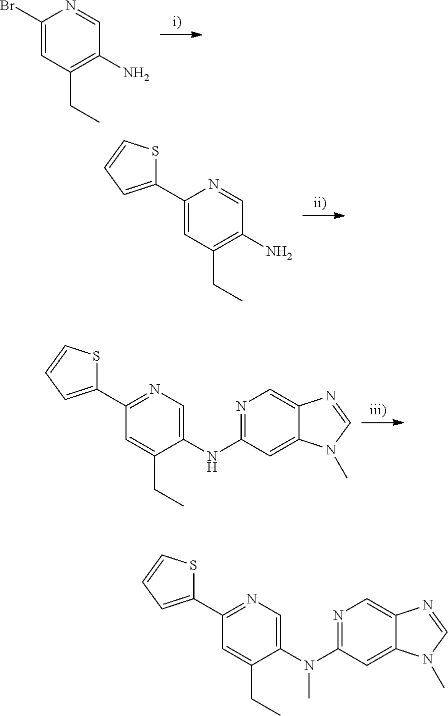

### Step i): 4-Ethyl-6-(thiophen-2-yl)pyridin-3-amine

A stirred mixture of 6-bromo-4-ethylpyridin-3-amine (200 mg, 1 mmol), 2-thiophene boronic acid (140 mg, 1.1 mmol), tetrakis(triphenylphosphine)palladiun(0) (25 mg) and sodium carbonate (1.5 mL of a 2 M solution in water) in dimethoxyethane (3 mL) was heated at 120° C. for 30 min using microwave irradiation. The reaction mixture was cooled to room temperature and EtOAc and water were added. The aqueous was extracted with EtOAc, the combined organics were dried (MgSO₄), filtered and concentrated in vacuo. The residue was purified by flash chromatography (0 to 10% 7N NH₃ solution in MeOH in EtOAc) to give the desired compound.

### Step ii): N-(4-Ethyl-6-(thiophen-2-yl)pyridin-3-yl)-1-methyl-1H-imidazo[4,5-c]pyridin-6-amine

A stirred mixture of 4-ethyl-6-(thiophen-2-yl)pyridin-3-amine (0.15 g, 0.71 mmol), 6-chloro-1-methyl-1H-imidazo[4,5-c]pyridine (0.12 g, 0.71 mmol), sodium tert-butoxide (175 mg), tris(dibenzylideneacetone)dipalladium(0) (23 mg) and 2,2′-bis(diphenylphosphino)-1,1′-binaphthyl (22 mg) was heated in 1,4-dioxane (5 mL) at 100° C. in a sealed tube for 1 d. The mixture was evaporated in vacuo and the residue was partitioned between DCM and water. The organic phase was separated and the aqueous phase extracted with DCM (×3), the combined organic phases were dried (MgSO₄) and evaporated in vacuo. The residue was purified by flash chromatography (7N NH₃ solution in MeOH in DCM, 0-10%) to give the desired compound.

### Step iii): N-(4-Ethyl-6-(thiophen-2-yl)pyridin-3-yl)-N,1-dimethyl-1H-imidazo[4,5-c]pyridin-6-amine

Sodium hydride (60% in mineral oil, 20 mg, 0.5 mmol) was added to a stirred solution of N-(4-ethyl-6-(thiophen-2-yl)pyridin-3-yl)-1-methyl-1H-imidazo[4,5-c]pyridin-6-amine (145 mg, 0.43 mmol) in DMF (4 mL) and stirred at room temperature for 20 min. A solution of iodomethane (50 μL, 0.8 mmol) in DMF (1 mL) was added and the solution was stirred at room temperature for 2 h. Water and DCM was added and the aqueous extracted with DCM. The combined organics were dried (hydrophobic frit) and concentrated in vacuo. The residue was purified by preparative HPLC to give the desired compound.

¹H NMR δ (ppm)(DMSO-d₆): 8.48 (1H, s, ArH), 8.31 (1H, s, ArH), 8.02 (1H, s, ArH), 7.92 (1H, s, ArH), 7.84 (1H, d, ArH), 7.64 (1H, d, ArH), 7.21-7.17 (1H, m, ArH), 6.44 (1H, s, ArH), 3.67 (3H, s, CH3), 3.39 (3H, s, CH3), 2.54-2.42 (2H, m, CH2), 1.15 (3H, t, CH3).

LCMS (10 cm_ESCI_Bicarb_MeCN) t_{R} 3.45 (min) m/z 350 (MH⁺).

### Compound 291: 3-{3-Ethyl-4-[methyl-(1-methyl-1H-imidazo[4,5-c]pyridin-6-yl)-amino]-benzylamino}-pyrrolidine-1-carboxylic acid tert-butyl ester

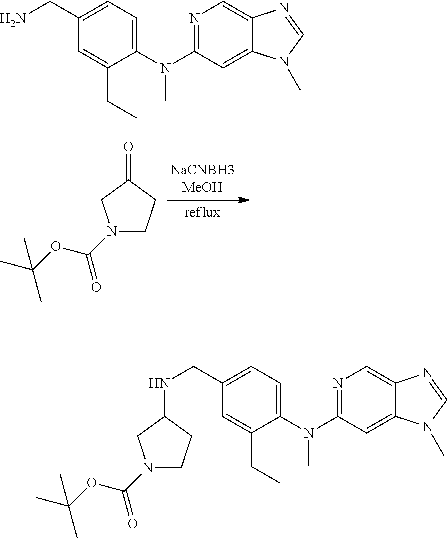

Intermediate 8 (500 mg) was dissolved in 10 mL dry MeOH. The ketone (1 equiv, 305 mg) and NaCNBH₃ (2 equiv, 210 mg) were added and the reaction mixture was stirred at reflux for 16 h. LCMS showed 100% desired compound. The reaction mixture was evaporated and the crude was purified by preparative HPLC.

### Compound 293: [2-Ethyl-4-(pyrrolidin-3-ylaminomethyl)-phenyl]-methyl-(1-methyl-1H-imidazo[4,5-d]pyridin-6-yl)-amine

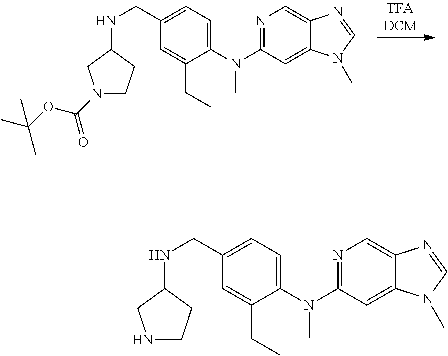 [SMILES: CCc1cc(CNC2CCN(C(=O)OC(C)(C)C)C2)ccc1N(C)c1cc2c(cn1)ncn2C ; CCc1cc(CNC2CCNC2)ccc1N(C)c1cc2c(cn1)ncn2C]

Compound 291 (50 mg) was dissolved in DCM (1 mL) and TFA was added (250 μL). The mixture was stirred for 2 h after which the mixture was evaporated under reduced pressure to yield the desired compound.

### Compound 295: 2-(3-ethyl-4-(methyl(1-methyl-1H-imidazo[4,5-c]pyridin-6-yl)amino)phenoxy)acetonitrile

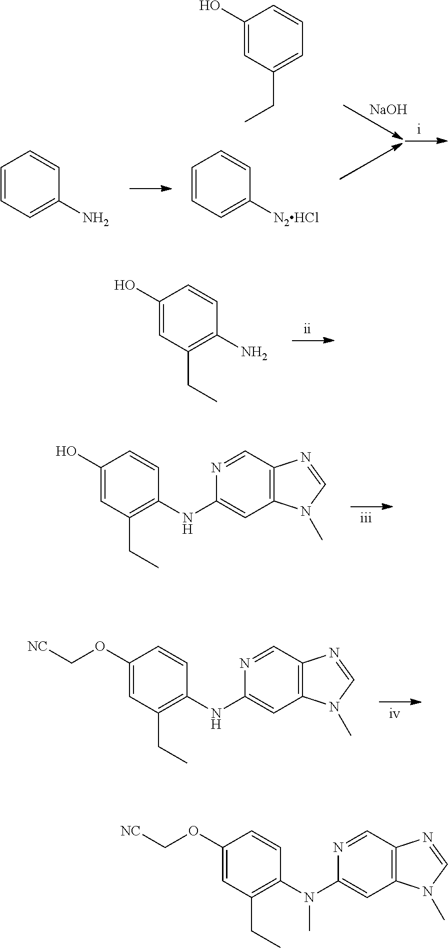

### Step i: 4-Amino-3-ethyl-phenol

Aniline (410 μL, 4.51 mmol, 1.1 equiv) was dissolved in water (3 mL) with concentrated HCl (755 μL, 9.43 mmol, 2.3 equiv) at 0° C. NaNO₂ (325 mg, 4.72 mmol, 1.15 equiv) was added and the mixture was stirred for 15 nm. The diazoted aniline was added to 3-Ethyl-phenol (500 μL, 4.10 mmol, 1.0 equiv) in 6% aqueous NaOH (5 mL) under vigourous stirring. After 5 nm, it was allowed to stand for 30 nm. LCMS showed formation of the azo dye. The precipitate was filtrated and washed with water (5×10 mL). The filtrate was acidified with H₂SO₄ (exothermic reaction, use an ice bath). The azo dye was extracted with diethyl ether (3×40 mL). The combined organic layer was dried over Na₂SO₄, filtered and concentrated in vacuo to afford the azo dye. The azo dye was diluted in MeOH (20 mL) and an aqueous solution of Na₂S₂O₄ (1M, 2.5 equiv) was added. The mixture was stirred at 65° C. for 3 h. The reaction was cooled down to room temperature. A precipitate formed, it was filtered and washed with MeOH. The filtrate was concentrated in vacuo and the residue was diluted in EtOAc and washed with H₂O (3×30 mL). The compound was then extracted in EtOAc (4×30 mL and the combined organic layer was dried over Na₂SO₄, filtered and concentrated in vacuo to afford the desired compound.

### Step ii: 3-Ethyl-4-(1-methyl-1H-imidazo[4,5-c]pyridin-6-ylamino)-phenol

4-Amino-3-ethyl-phenol (100 mg, 0.73 mmol, 1.0 equiv), 6-Chloro-1-methyl-1H-imidazo[4,5-c]-pyridine (134 mg, 0.80 mmol, 1.1 equiv), Cs₂CO₃ (714 mg, 2.19 mmol, 3.0 equiv), BINAP (46 mg, 0.07 mmol, 0.1 equiv), and Pd₂(dba)₃ (33 mg, 0.04 mmol, 0.05 equiv) were dissolved in dry dioxane (5 mL) under N₂ atmosphere. The mixture was sonicated for 5 nm under N₂ flow and then refluxed for 20 h. Full conversion was observed by LCMS. It was then cooled down and concentrated to dryness, diluted in DCM and washed with water (3×20 mL). The aqueous layer was extracted with DCM (3×30 mL) and the combined organic layer was dried over Na₂SO₄, filtered and concentrated to dryness. Purification was performed by column chromatography using the following eluant: EtOAc/Petroleum Ether (1:1) to EtOAc (over 5 CV), MeOH/EtOAc (1:19) (10 CV), MeOH/EtOAc (1:9) (10 CV) to afford the desired compound.

### Step iii: [3-Ethyl-4-(1-methyl-1H-imidazo[4,5-c]pyridin-6-ylamino)-phenoxy]-acetonitrile

3-Ethyl-4-(1-methyl-1H-imidazo[4,5-c]pyridin-6-ylamino)-phenol (45 mg, 0.17 mmol, 1.0 equiv) was dissolved in dry THF (5 mL) under N₂ atmosphere. Cs₂CO₃ (111 mg, 0.34 mmol, 2 equiv) was added and the mixture was stirred for 10 nm. Chloro-acetonitrile (12 μL, 0.19 mmol, 1.1 equiv) was added and the mixture was heated up to 40° C. for 50 h. LCMS showed full conversion toward the desired compound. The reaction was stopped, cooled down to room temperature and concentrated in vacuo. The residue was diluted in DCM and washed with water (3×30 mL). The compound was extracted with DCM (3×30 mL) and the combined organic layer was dried over Na₂SO₄, filtered and concentrated in vacuo to afford the crude material which was used as such.

### Step iv: 2-(3-ethyl-4-(methyl(1-methyl-1H-imidazo[4,5-c]pyridin-6-yl)amino)phenoxy)acetonitrile

[3-Ethyl-4-(1-methyl-1H-imidazo[4,5-c]pyridin-6-ylamino)-phenoxy]-acetonitrile (52 mg, 0.17 mmol, 1.0 equiv) was dissolved in dry THF (5 mL) under N₂ atmosphere. NaH (60% in mineral oil) (20 mg, 0.51 mmol, 3 equiv) was added and the mixture was stirred for 10 nm. MeI (16 μL, 0.25 mmol, 1.5 equiv) was added and the mixture was stirred for 16 h. The reaction was quenched with water, concentrated in vacuo and the residue was diluted in DCM. The solution was washed with water (3×30 mL), the compound was extracted with DCM (3×30 mL) and the combined organic layer was dried over Na₂SO₄, filtered and concentrated at rotavap to afford 72 mg of crude material. Purification by column chromatography using the following eluant: EtOAc (15 CV), MeOH/EtOAc (1:19) (15 CV), MeOH/EtOAc (1:9) (15 CV) led to the desired compound.

### Compound 296: 1-cyclopropyl-N-(2-ethylphenyl)-N-methyl-1H-imidazo[4,5-c]pyridin-6-amine

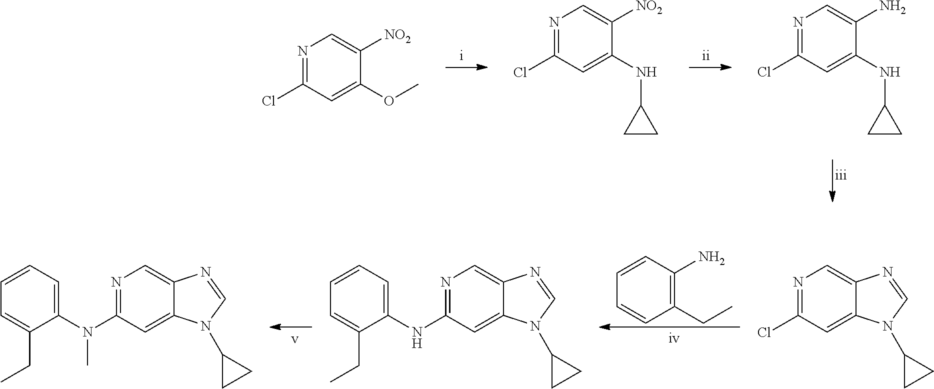

### Step 1): (2-chloro-5-nitro-pyridin-4-yl)-cyclopropyl-amine

To a solution of 2-chloro-4-methoxy-5-nitropyridine (0.5 g, 2.67 mmol, 1.0 equiv) in dry THF (3.0 mL) was added cyclopropylamine (0.31 g, 5.32 mmol, 2.0 equiv). The reaction was stirred for 2 h at room temperature. The reaction mixture was diluted with water and the compound was extracted with ethyl acetate. The organic layer was dried over MgSO₄, filtered and removed under vacuum to afford the desired compound.

### Step): 6-chloro-N-4-cyclopropyl-pyridine-3,4-diamine

To a solution of (2-chloro-5-nitro-pyridin-4-yl)-cyclopropyl-amine (0.57 g, 2.67 mmol, 1.0 equiv) in acetic acid (10 mL) at 50° C., iron powder (0.60 g, 10.74 mmol, 4.0 equiv.) was added portion wise to allow the correct stirring. The reaction mixture was stirred at 80° C. for 24 h. The mixture was cooled down to room temperature, and filtered through Celite. The solid pad was washed with ethyl acetate. The filtrate was concentrated under vacuum, and the residual mass was neutralized with saturated aqueous solution of NaHCO₃. The compound was extracted with ethyl acetate. The organic layer was washed with water, dried over MgSO₄, filtered and removed the solvent under vacuum to afford the desired compound.

Step iii): 6-Chloro-1-cyclopropyl-1H-imidazo[4,5-c]pyridine

To a solution of 6-chloro-N4-cyclopropyl-pyridine-3,4-diamine (0.46 g, 2.67 mmol, 1.0 equiv.) in trimethylorthoformate (3 mL) was added formic acid (0.2 mL), and the reaction was stirred at 100° C. for 18 h. The reaction was cooled down to room temperature, diluted with ethyl acetate, and washed with water. The organic layer was dried over MgSO₄, filtered and removed under vacuum to afford the desired compound.

### Step iv): (1-cyclopropyl-1H-imidazo[4,5-c]pyridin-6-yl)-(2-ethyl-phenyl)-amine

6-Chloro-1-cyclopropyl-1H-imidazo[4,5-c]pyridine (0.46 g, 2.67 mmol, 1.0 equiv.), 2-ethylaniline (0.50 mL, 4.01 mmol, 1.5 equiv.), and cesium carbonate (2.67 g, 8.01 mmol, 3.0 equiv.) were dissolved in dry dioxane (10.0 mL), and the mixture was purged with nitrogen under sonication for min. The palladium catalyst (0.50 g, 0.54 mmol, 0.2 equiv.) and the ligand (0.68 g, 1.08 mmol, 0.4 equiv.) were added to the reaction mixture and purged again with nitrogen under sonication for 5 min. The reaction mixture was stirred at 110° C. for 15 h. The reaction was cooled down to room temperature, diluted with ethyl acetate, and washed with water. The organic layer was dried over MgSO₄, filtered and removed under vacuum to afford the desired compound.

### Step v): 1-cyclopropyl-N-(2-ethylphenyl)-N-methyl-1H-imidazo[4,5-c]pyridin-6-amine

To a solution of (1-cyclopropyl-1H-imidazo[4,5-c]pyridin-6-yl)-(2-ethyl-phenyl)-amine (0.71 g, 2.67 mmol, 1.0 equiv.) in THF (3.0 mL) at 0° C., sodium hydride 60% dispersion in mineral oil (0.13 g, 3.20 mmol, 1.2 equiv.) was added portion wise, and the mixture was stirred for 30 min. Iodomethane (0.21 mL, 3.20 mmol, 1.2 equiv.) was added and the mixture was allowed to warm to room temperature. The reaction was stirred for 15 h. The reaction was diluted with ethyl acetate and washed with water. The organic layer was dried over MgSO₄, filtered and removed under vacuum. The crude of the reaction was purified by semipreparative LCMS (XSelect CSH Prep OBD Column, C18 19×100 mm 5 μm (Waters); gradient: acetonitrile/0.1% formic acid in water, from 5% to 30% in 5 min) to afford the desired product. ESI-MS: 293.0 [M+1].

### Compound 297: 1-ethyl-N-(2-ethylphenyl)-N-methyl-1H-imidazo[4,5-c]pyridin-6-amine

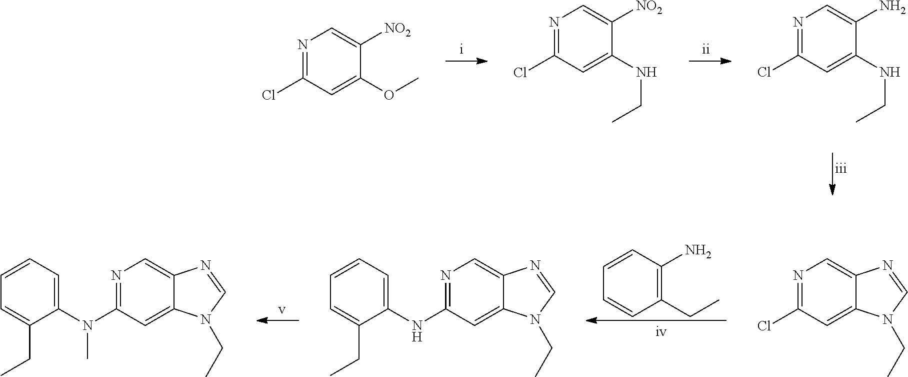

### Step i): 2-chloro-4-ethylamino-5-nitropyridine

To a solution of 2-chloro-4-methoxy-5-nitropyridine (0.5 g, 2.67 mmol, 1.0 equiv) in dry THF (3.0 mL) was added a solution of ethylamine 70% in water (0.342 mL, 5.32 mmol, 2.0 equiv). The reaction was stirred for 2 h at room temperature. The reaction mixture was diluted with water and the compound was extracted with ethyl acetate. The organic layer was dried over MgSO₄, filtered and removed under vacuum to afford the desired compound.

### Step ii): 5-amino-2-chloro-4-ethylaminopyridine

To a solution of 2-chloro-4-ethylamino-5-nitropyridine (0.54 g, 2.67 mmol, 1.0 equiv) in acetic acid (10 mL) at 50° C., iron powder (0.60 g, 10.74 mmol, 4.0 equiv.) was added portionwise to allow the correct stirring. The reaction mixture was stirred at 80° C. for 24 h. The mixture was cooled down to room temperature, and filtered through Celite. The solid pad was washed with ethyl acetate. The filtrate was concentrated under vacuum, and the residual mass was neutralized with saturated aqueous solution of NaHCO₃. The compound was extracted with ethyl acetate. The organic layer was washed with water, dried over MgSO₄, filtered and removed the solvent under vacuum to afford the desired compound.

### Step iii): 6-Chloro-1-ethyl-1H-imidazo[4,5-c]pyridine

To a solution of 5-amino-2-chloro-4-ethylaminopyridine (0.46 g, 2.67 mmol, 1.0 equiv.) in trimethylorthoformate (3 mL) was added formic acid (0.2 mL), and the reaction was stirred at 100° C. for 18 h. The reaction was cooled down to room temperature, diluted with ethyl acetate, and washed with water. The organic layer was dried over MgSO₄, filtered and removed under vacuum to afford the desired compound.

### Step iv): (1-ethyl-1H-imidazo[4,5-c]pyridin-6-yl)-(2-ethyl-phenyl)-amine

6-Chloro-1-ethyl-1H-imidazo[4,5-c]pyridine (0.23 g, 1.27 mmol, 1.0 equiv.), 2-ethylaniline (0.158 mL, 1.91 mmol, 1.5 equiv.), and cesium carbonate (1.24 g, 3.81 mmol, 3.0 equiv.) were dissolved in dry dioxane (2.0 mL), and the mixture was purged with nitrogen under sonication for 5 min. The palladium catalyst (0.11 g, 0.12 mmol, 0.1 equiv.) and the ligand (0.15 g, 0.24 mmol, 0.2 equiv.) were added to the reaction mixture and purged again with nitrogen under sonication for 5 min. The reaction mixture was stirred at 110° C. for 15 h. The reaction was cooled down to room temperature, diluted with ethyl acetate, and washed with water. The organic layer was dried over MgSO₄, filtered and removed under vacuum to afford the compound.

### Step v): 1-ethyl-N-(2-ethylphenyl)-N-methyl-1H-imidazo[4,5-c]pyridin-6-amine

To a solution of (1-ethyl-1H-imidazo[4,5-c]pyridin-6-yl)-(2-ethyl-phenyl)-amine (0.34 g, 1.27 mmol, 1.0 equiv.) in THF (2.0 mL) at 0° C., sodium hydride 60% dispersion in mineral oil (0.062 g, 1.54 mmol, 1.2 equiv.) was added portion wise, and the mixture was stirred for 30 min. Iodomethane (0.098 mL, 1.54 mmol, 1.2 equiv.) was added and the mixture was allowed to warm to room temperature. The reaction was stirred for 15 h. The reaction was diluted with ethyl acetate and washed with water. The organic layer was dried over MgSO₄, filtered and removed under vacuum. The crude of the reaction was purified by semipreparative LCMS (XBridge Prep OBD Column, C18 19×100 mm 5 μm (Waters); gradient: acetonitrile/0.5% NH3 in water, from 30% to 55% in 5 min) to afford the desired product. ¹H-NMR (400 MHz, DMSO) δ (ppm): 8.48 (1H, d, ArH), 8.03 (1H, s, ArH), 7.39 (1H, m, ArH), 7.29 (2H, m, ArH), 7.18 (1H m, ArH), 6.11 (1H, d, ArH), 4.00 (2H, q, CH2), 3.34 (3H, s, CH3), 2.44 (2H, q, CH2), 1.25 (3H, t, CH3), 1.08 (3H, t, CH3). ESI-MS: 280.9 [M+1].

### Compound 300: {4-[(1,1-Dioxo-tetrahydro-thiophen-3-ylamino)-methyl]-2-ethyl-phenyl}-methyl-(1-methyl-1H-imidazo[4,5-c]pyridin-6-yl)-amine

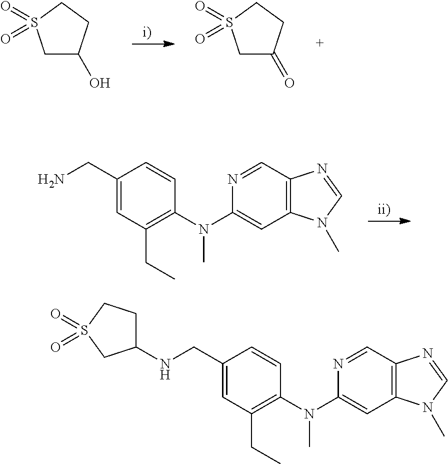 [SMILES: O=S1(=O)CCC(O)C1 ; O=C1CCS(=O)(=O)C1 ; CCc1cc(CN)ccc1N(C)c1cc2c(cn1)ncn2C ; CCc1cc(CNC2CCS(=O)(=O)C2)ccc1N(C)c1cc2c(cn1)ncn2C]

### Step (i) 1,1-Dioxo-tetrahydro-thiophen-3-one

MnO₂ (35 equiv, 2.2 g) was added to the alcohol (1 equiv, 100 mg) in DCM (20 mL) and the reaction was stirred at room temperature for 16 h. The reaction mixture was filtered and concentrated in vacuo. The residue was used in the next step without further purification.

### Step (ii): {4-[(1,1-Dioxo-tetrahydro-thiophen-3-ylamino)-methyl]-2-ethyl-phenyl}-methyl-(1-methyl-1H-imidazo[4,5-c]pyridin-6-yl)-amine

### Compound 181 (1 equiv, 100 mg) was dissolved in 2 mL dry MeOH. The ketone (1.2 equiv) and NaCNBH₃ (6 equiv, 126 mg) were added and the reaction mixture was stirred at reflux for 3 days. The reaction mixture was concentrated and the crude was purified by preparative HPLC.

### Compound 301: 5-{4-Ethyl-5-[methyl-(1-methyl-1H-imidazo[4,5-c]pyridin-6-yl)-amino]-pyridin-2-yl}-thiophene-2-carbonitrile

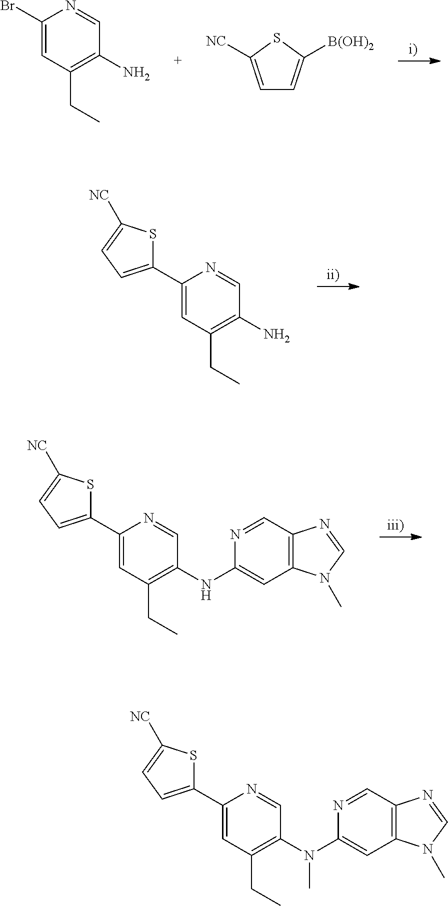

### Step i): (5-(5-Amino-4-ethyl-pyridin-2-yl)-thiophene-2-carbonitrile

A mixture of 4-bromo-2-ethylaniline (1 equiv, 100 mg), the boronic acid (1.1 equiv, 84 mg), potassium carbonate (3 equiv, 207 mg) and [1,1′-bis(diphenylphosphino)ferrocene]dichloropalladium(II) (0.1 equiv, 41 mg) in 1,4-dioxane (4 mL) and water (1 mL) was heated at 80° C. for 16 h. After completion of the reaction, the reaction mixture was concentrated in vacuo and purified by silica chromatography (EtOAc/petrol ether; 20:80 to 60:40) to give the desired product. LC-MS: [M+H]⁺230.0.

### Step (ii): 5-[4-Ethyl-5-(1-methyl-1H-imidazo[4,5-c]pyridin-6-ylamino)-pyridin-2-yl]-thiophene-2-carbonitrile

A mixture of the aniline (1 equiv, 40 mg), 6-chloro-1-methyl-1H-imidazo[4,5-c]pyridine (1.1 equiv, 32 mg), BINAP (0.2 equiv, 44 mg), Cs₂CO₃ (3 equiv, 325 mg) and tris(dibenzylideneacetone)dipalladium(0) (0.1 equiv, 24 mg) in dry 1,4-dioxane (10 mL), degassed under (N₂), was heated at reflux temperature for 16 h. The solution was concentrated in vacuo and purified by silica chromatography to give the desired product.

LC-MS: [M+H]⁺361.0.

Step 5-{4-Ethyl-5-[methyl-(1-methyl-1H-imidazo[4,5-c]pyridin-6-yl)-amino]-pyridin-2-yl}-thiophene-2-carbonitrile

Sodium hydride (60% in mineral oil, 2 equiv, 7 mg) was added to a solution of the amine (1 equiv, 33 mg) in THF (5 mL) and the mixture was stirred for 5 min at room temperature. Iodomethane (2 equiv, 14 μL) was added and the solution stirred for 16 h. The resulting mixture was diluted in DCM, washed with brine, dried over MgSO₄ and concentrated to dryness. The residue was purified by silica chromatography to give the desired product.

### Compound 302: (5-{4-Ethyl-5-[methyl-(1-ethyl-1H-imidazo[4,5-c]pyridin-6-yl)-amino]-pyridin-2-yl}-thiophen-2-ylmethyl)-carbamic acid tert-butyl ester

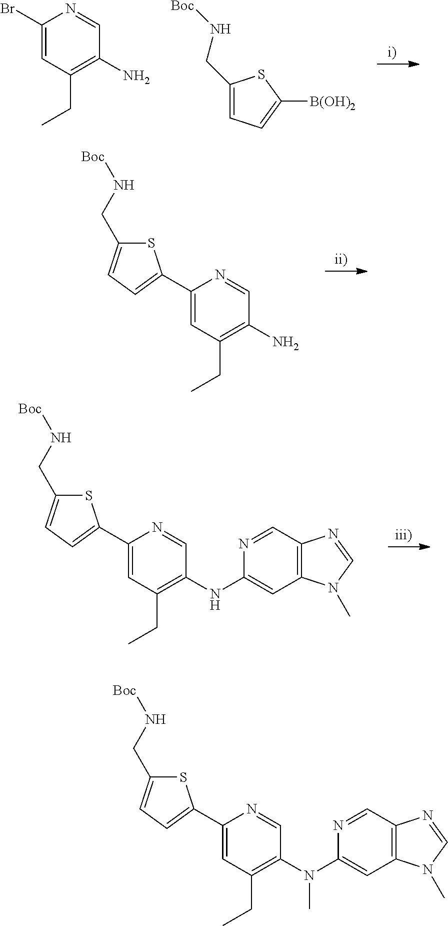

### Step i) and ii): N-(2-Methoxy-4-(thiophen-2-yl)phenyl)-1-methyl-1H-imidazo[4,5-c]pyridin-6-amine {5-[4-Ethyl-5-(1-methyl-1H-imidazo[4,5-c]pyridin-6-ylamino)-pyridin-2-yl]-thiophen-2-ylmethyl}-carbamic acid tert-butyl ester

The same procedures described for the preparation of compound 301 are used.

### Step (iii): (5-{4-Ethyl-5-[methyl-(1-ethyl-1H-imidazo[4,5-c]pyridin-6-yl)-amino]-pyridin-2-yl}-thiophen-2-ylmethyl)-carbamic acid tert-butyl ester

Sodium hydride (60% in mineral oil, 3 equiv, 10 mg) was added to a solution of the amine (1 equiv, 38 mg) in THF (5 mL) and the mixture was stirred for 5 min at room temperature. Iodomethane (1.1 equiv, 6 μL) was added and the solution stirred for 16 h. The mixture was diluted in DCM, washed with brine, dried over MgSO₄ and concentrated to dryness to give a mixture of both mono- and di-methylated products that were purified by preparative HPLC.

### Compound 303: [6-(5-Aminomethyl-thiophen-2-yl)-4-ethyl-pyridin-3-yl]-methyl-(1-methyl-1H-imidazo[4,5-c]pyridin-6-yl)-amine

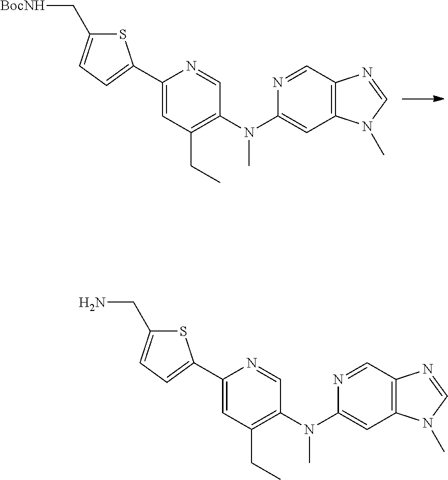 [SMILES: CCc1cc(-c2ccc(CNC(=O)OC(C)(C)C)s2)ncc1N(C)c1cc2c(cn1)ncn2C ; CCc1cc(-c2ccc(CN)s2)ncc1N(C)c1cc2c(cn1)ncn2C]

The Boc-protected amine (1 equiv, 5 mg) was dissolved in DCM (0.5 mL) and TFA was added (0.5 mL). The mixture was stirred for 5 min after which the mixture was concentrated in vacuo to give the desired compound.

### Compound 307: [5-(4-Chloro-phenyl)-2-ethyl-2H-pyrazol-3-yl]-methyl-(1-methyl-1H-imidazo[4,5-c]pyridin-6-yl)-amine

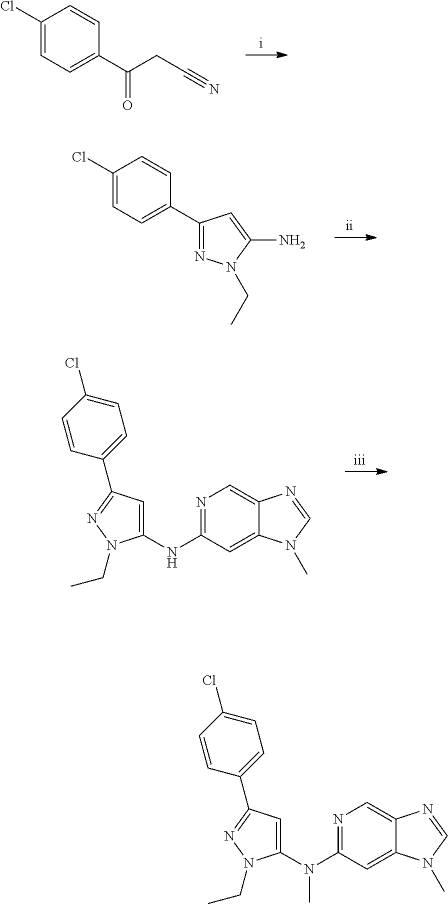 [SMILES: N#CCC(=O)c1ccc(Cl)cc1 ; CCn1nc(-c2ccc(Cl)cc2)cc1N ; CCn1nc(-c2ccc(Cl)cc2)cc1Nc1cc2c(cn1)ncn2C ; CCn1nc(-c2ccc(Cl)cc2)cc1N(C)c1cc2c(cn1)ncn2C]

### Step i): 5-(4-Chloro-phenyl)-2-ethyl-2H-pyrazol-3-ylamine

A solution of 3-(4-Chloro-phenyl)-3-oxo-propionitrile (1 equiv, 126 mg) and ethyl hydrazine oxalate (1 equiv, 106 mg) in a 0.2 M solution of HCl in MeOH (5 mL, made from a 5-6 M HCl in i-PrOH) was heated at reflux for 24 h. After completion of the reaction, the mixture was cooled at room temperature, basified with aq. 1 M NaOH (pH 12) and extracted with EtOAc (3×). The combined organics was dried (Na₂SO₄), filtered and concentrated in vacuo to give the desired product.

### Step ii) and iii): [5-(4-Chloro-phenyl)-2-ethyl-2H-pyrazol-3-yl]-methyl-(1-methyl-1H-imidazo[4,5-c]pyridin-6-yl)-amine

The same conditions described for the preparation of compound 301 are used.

### Compound 308: 3-Cyanomethyl-3-{4-ethyl-3-[methyl-(1-methyl-1H-imidazo[4,5-c]pyridin-6-yl)-amino]-pyrazol-1-yl}-azetidine-1-carboxylic acid tert-butyl ester

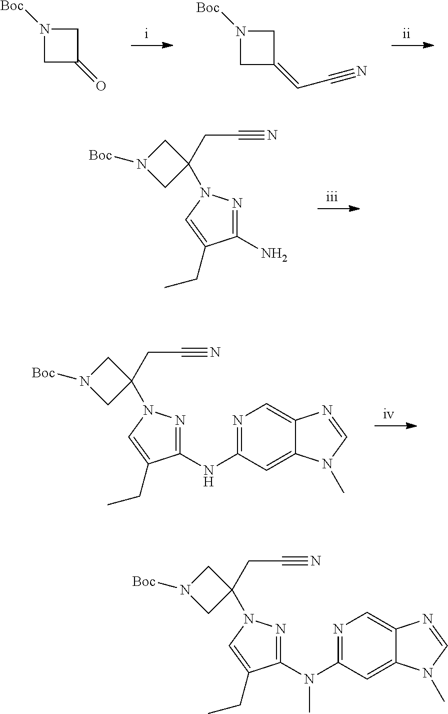 [SMILES: CC(C)(C)OC(=O)N1CC(=O)C1 ; CC(C)(C)OC(=O)N1CC(=CC#N)C1 ; CCc1cn(C2(CC#N)CN(C(=O)OC(C)(C)C)C2)nc1N ; CCc1cn(C2(CC#N)CN(C(=O)OC(C)(C)C)C2)nc1Nc1cc2c(cn1)ncn2C ; CCc1cn(C2(CC#N)CN(C(=O)OC(C)(C)C)C2)nc1N(C)c1cc2c(cn1)ncn2C]

### Step i): 3-Cyanomethylene-azetidine-1-carboxylic acid tert-butyl ester

Cyanomethyl-phosphonic acid diethyl ester (1.1 eq, 6.3 mL) was slowly added to a solution of NaH (60% in mineral oil, 0.98 equiv, 1.4 g) in dry THF (250 mL) under N₂ at 0° C. The mixture was stirred for 30 min at room temperature, cooled down to 0° C. and 3-oxo-azetidine-1-carboxylic acid tert-butyl ester (1 equiv, 6.1 g) was added. The reaction was stirred at room temperature until completion after what the mixture was concentrated in vacuo and purified by silica chromatography (EtOAc/petrol ether; 10:90 to 30:70) to give the desired compound.

### Step ii): 3-(3-Amino-4-ethyl-pyrazol-1-yl)-3-cyanomethyl-azetidine-1-carboxylic acid tert-butyl ester

A solution of 4-ethyl-1H-pyrazol-3-ylamine oxalate (1 equiv, 1.85 g), 3-cyanomethylene-azetidine-1-carboxylic acid tert-butyl ester (1.1 equiv, 1.92 g), DBU (3 equiv, 4.2 mL) in CH₃CN (40 mL) was stirred at room temperature for 16 h. The reaction mixture was concentrated and purified by silica chromatography (EtOAc/petrol ether; 40:60 to 100:0) to give the desired compound.

### Step iii) and iv).: [5-(4-Chloro-phenyl)-2-ethyl-2H-pyrazol-3-yl]-methyl-(1-methyl-1H-imidazo[4,5-c]pyridin-6-yl)-amine

The same conditions described for the preparation of compound 301 are used.

### Compound 310: (3-{4-Ethyl-3-[methyl-(1-methyl-1H-imidazo[4,5-c]pyridin-6-yl)-mino]-pyrazol-1-yl}-1-methanesulfonyl-azetidin-3-yl)-acetonitrile

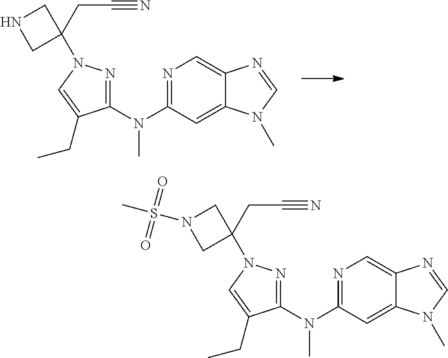

Mesityl chloride (1.5 equiv, 26 μL) was added to a solution of the azetidine (1 equiv, 78 mg) and NEt₃ (2.1 equiv, 64 μL) in DCM (4 mL) and the reaction mixture was stirred at room temperature for 2 h. The solution was concentrated and purified by preparative HPLC to give the desired compound.

### Compound 311: [4-Ethyl-6-(5-methyl-thiophen-2-yl)-pyridin-3-yl]-methyl-(1-methyl-1H-imidazo[4,5-c]pyridin-6-yl)-amine

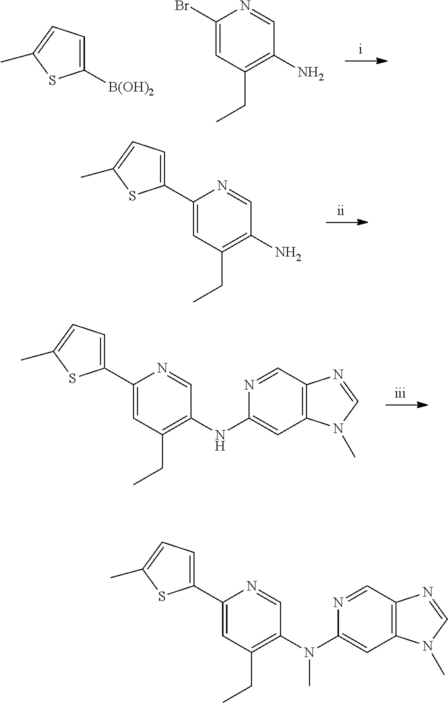

### Step i): 4-Ethyl-6-(5-methyl-thiophen-2-yl)-pyridin-3-ylamine

A mixture of the boronic acid (1.1 equiv, 155 mg), the bromopyridine (1 equiv, 200 mg), Pd(PPh₃)₄ (0.02 equiv, 23 mg) in DME (3 mL) and aq. 2M Na₂CO₃ (1.5 mL) was irradiated in microwave Biotage Initiator at 130° C. for 30 min. The resulting mixture was diluted with water (10 mL) and extracted with EtOAc (3×15 mL). The residue was purified by preparative HPLC to give the desired compound.

Step ii) [4-Ethyl-6-(5-methyl-thiophen-2-yl)-pyridin-3-yl]-(1-methyl-1H-imidazo[4,5-c]pyridin-6-yl)-amine

A mixture of the aniline (1 equiv, 60 mg), the 6-chloro-1-methyl-1H-imidazo[4,5-c]pyridine (1 equiv, 41 mg), 2-dicyclohexylphosphino-2′,4′,6′-triisopropylbiphenyl (0.1 equiv, 13 mg), tris(dibenzylideneacetone)dipalladium (0) (0.1 equiv, 25 mg) and Cs₂CO₃ (2 equiv 179 mg) in dioxane (3.5 mL) was irradiated in microwave Biotage Initiator at 150° C. for 45 min. The resulting mixture was diluted with water (10 mL) and extracted with EtOAc (3×15 mL). The residue was purified by silica chromatography (EtOAc/cyclohexane; 0:100 to 10:90) to give the desired compound.

Step (iii) [4-Ethyl-6-(5-methyl-thiophen-2-yl)-pyridin-3-yl]-(1-methyl-1H-imidazo[4,5-c]pyridin-6-yl)-amine

NaH (1 equiv, 5.7 mg) was added to the amine (1 equiv, 50 mg) in THF (2 mL) and stirred at room temperature for 15 min. MeI (1 equiv, 9 μL) was then added and resulting mixture was stirred at room temperature for 5 h. The resulting mixture was concentrated and purified by silica chromatography (MeOH/DCM; 0:100 to 10:90) to give the desired compound.

### Compound 312: [4-Methoxy-6-(1,3,5-trimethyl-1H-pyrazol-4-yl)-pyridin-3-yl]-methyl-(1-ethyl-1H-imidazo[4,5-c]pyridin-6-yl)-amine

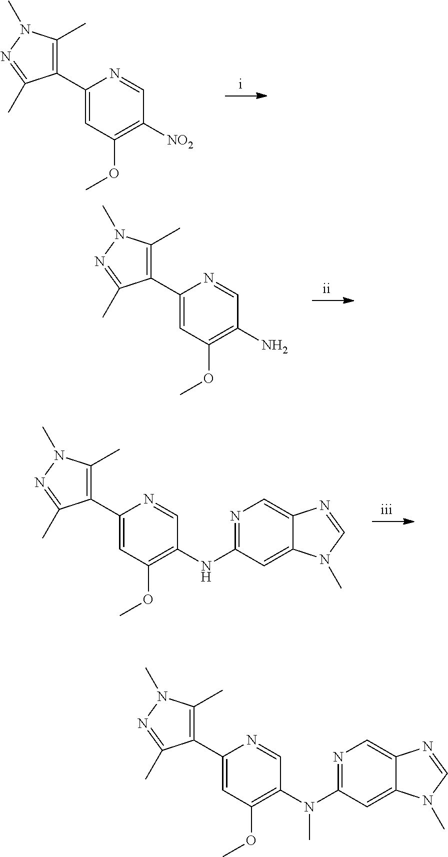

### Step (i): 4-Methoxy-6-(1,3,5-trimethyl-1H-pyrazol-4-yl)-pyridin-3-ylamine

A sealed vessel containing a mixture of 4-methoxy-5-nitro-2-(1,3,5-trimethyl-1H-pyrazol-4-yl)-pyridine (1 equiv, 640 mg), iron powder (5 equiv, 681 mg) and NH₄Cl (1.5 equiv, 196 mg) in THF (4.5 mL), EtOH (4.5 mL) and water (1.5 mL) was heated at 100° C. for 3 h. Water was added, the reaction mixture was extracted with EtOAc (3×) and the combined organics were dried and concentrated. The residue was dissolved in MeOH and purified on a SCX column to give the desired compound.

### Step (ii). (4-Methoxy-6-thiophen-2-yl-pyridin-3-yl)-(1-methyl-1H-imidazo[4,5-c]pyridin-6-yl)-amine

A degassed mixture of the amine (1 equiv, 200 mg), 6-chloro-1-methyl-1H-imidazo[4,5-c]pyridine (1 equiv, 144 mg), Cs₂CO₃ (2 equiv, 561 mg), Xphos (0.15 equiv, 62 mg) and Pd₂(dba)₃ (0.1 equiv, 79 mg) in dry 1.4-dioxane (5 mL) was heated at 120° C. for 5 h. Water (10 mL) was added and the reaction mixture was extracted with EtOAc (3×). The combined organics were dried, concentrated and the residue was purified by silica chromatography (EtOAc/cyclohexane; 0:100 to 50:50) to give the desired compound.

### Step (iii): [4-Methoxy-6-(1,3,5-trimethyl-1H-pyrazol-4-yl)-pyridin-3-yl]-methyl-(1-ethyl-1H-imidazo[4,5-c]pyridin-6-yl)-amine

Sodium hydride (1 equiv, 5.5 mg) was added to the amine (1 equiv, 50 mg) in THF (2 mL) and stirred at room temperature for 15 min. MeI (1 equiv, 9 μL) was then added and resulting mixture was stirred at room temperature for 5 h. The mixture was diluted in water, extracted with EtOAc (3×) and the resulting residue was purified by silica chromatography (EtOAc/cyclohexane; 0:100 to 50:50) to give the desired compound.

### Compound 313: (4-Methoxy-6-morpholin-4-yl-pyridin-3-yl)-methyl-(1-methyl-1H-imidazo[4,5-c]pyridine-6-yl)-amine

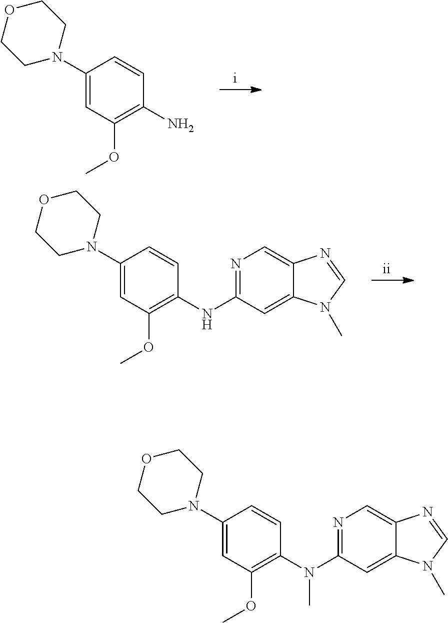

Synthesised following procedure used for compound 312 (step (ii) and (iii)).

### Compound 314: [2-Methoxy-5-(piperidine-1-sulfonyl)-phenyl]-methyl-(1-methyl-1H-imidazo[4,5-d]pyridin-6-yl)-amine

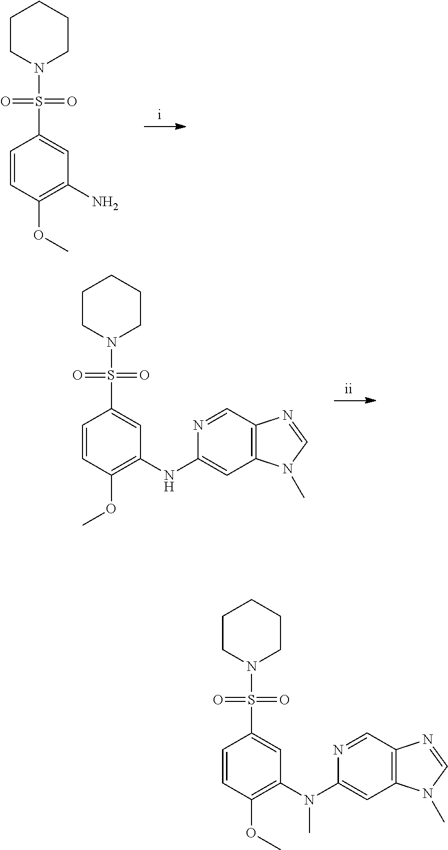

### Step i): 4-Ethyl-6-(5-methyl-thiophen-2-yl)-pyridin-3-ylamine

Synthesised following the same conditions used for the preparation of compound 312 (Step (ii)/(iii))

### Compound 315: [2-Methoxy-4-(morpholine-4-sulfonyl)-phenyl]-methyl-(1-methyl-1H-imidazo[4,5-d]pyridin-6-yl)-amine

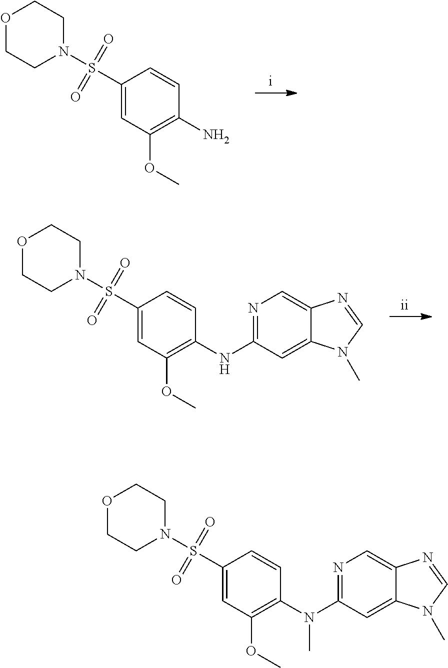 [SMILES: COc1cc(S(=O)(=O)N2CCOCC2)ccc1N ; COc1cc(S(=O)(=O)N2CCOCC2)ccc1Nc1cc2c(cn1)ncn2C ; COc1cc(S(=O)(=O)N2CCOCC2)ccc1N(C)c1cc2c(cn1)ncn2C]

Synthesised following the same conditions used for the preparation of compound 312 (Step ii)/(iii))

### Compound 316: 3-{4-Ethyl-3-[methyl-(1-methyl-1H-imidazo[4,5-c]pyridin-6-yl)-amino]-pyrazol-1-yl}-propionitrile

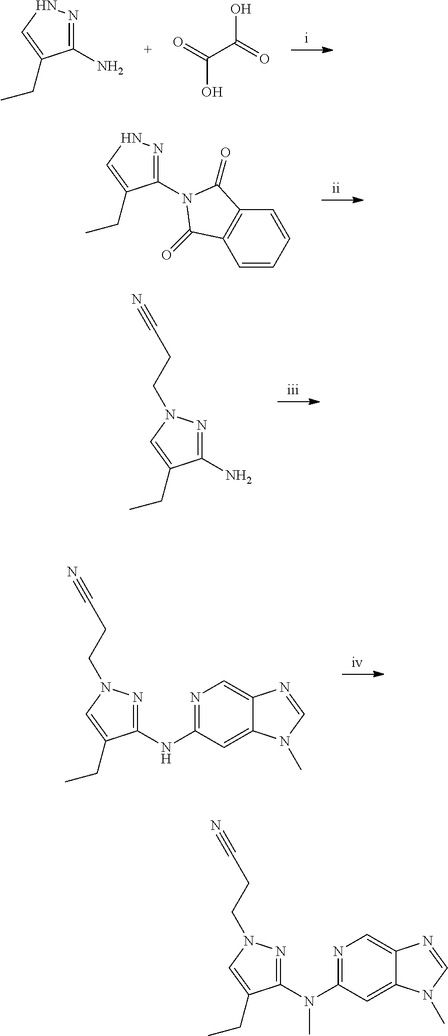

### Step i): 2-(4-Ethyl-1H-pyrazol-3-yl)-isoindole-1,3-dione

A mixture of 3-amino-4-ethyl pyrazole oxalate (1 equiv, 1.5 g) and phtalic anhydride (1 equiv, 1.16 g) in glacial acetic acid (12 mL) was heated at 120° C. for 3 h. Once the mixture was cooled at room temperature, water was added and the resulting precipitate was collected by filtration. The residue was then purified by silica chromatography (MeOH/DCM) to give the desired compound.

### Step ii): 3-(3-Amino-4-ethyl-pyrazol-1-yl)-propionitrile

DBU (0.5 equiv, 155 μL) was added to a solution of the pyrazole (1 equiv, 500 mg) and acrylonitrile (1.5 equiv, 206 μL) in dry acetonitrile (3 mL) and the reaction mixture was stirred at room temperature for 2 h after what the solvent was removed in vacuo. The residue was then redissolved in MeOH (10 mL), hydrazine hydrate (3 equiv, 301 μL) was added and the solution was heated at reflux for 1 h. The resulting mixture was concentrated and the residue purified by silica chromatography (MeOH/DCM; 2:98 to 15:85) to give the desired product.

Step iii): 3-[4-Ethyl-3-(1-methyl-1H-imidazo[4,5-c]pyridin-6-ylamino)-pyrazol-1-yl]-propionitrile

A degassed mixture of the aminopyrazole (1.1 equiv, 90 mg), 6-chloro-1-methyl-1H-imidazo[4,5-c]pyridine (1 equiv, 84 mg), Pd₂(dba)₃ (0.1 equiv, 46 mg), xantphos (0.3 equiv, 86 mg) and Cs₂CO₃ (2 equiv, 324 mg) in dry dioxane (3 mL) under inert atmosphere was heated at 110° C. for 36 h. The reaction mixture was filtered over a pad of Celite, washed with MeOH and concentrated. The residue was purified by silica chromatography (MeOH/DCM; 2:98 to 30:70) to give the desired compound.

### Step iv): 3-{4-Ethyl-3-[methyl-(1-methyl-1H-imidazo[4,5-c]pyridin-6-yl)-amino]pyrazol-1-yl}-propionitrile

Methyl iodide (1.3 equiv, 9.5 μL) was added to a mixture of NaH (1.3 equiv, 6.1 mg) and the amine (1 equiv, 35 mg) in THF (1.5 mL) at 0° C. The mixture was warmed up to room temperature and stirred for 16 h. Water was added to the reaction mixture and the solution was extracted with EtOAc (3×), dried and concentrated. The residue was purified by silica chromatography (MeOH/DCM; 3:97 to 30:70) to give the desired compound.

### Compound 317: N-{4-Ethyl-5-[methyl-(1-methyl-1H-imidazo[4,5-c]pyridin-6-yl)-amino]-pyridin-2-ylmethyl}-N-methyl-methane sulfonamide

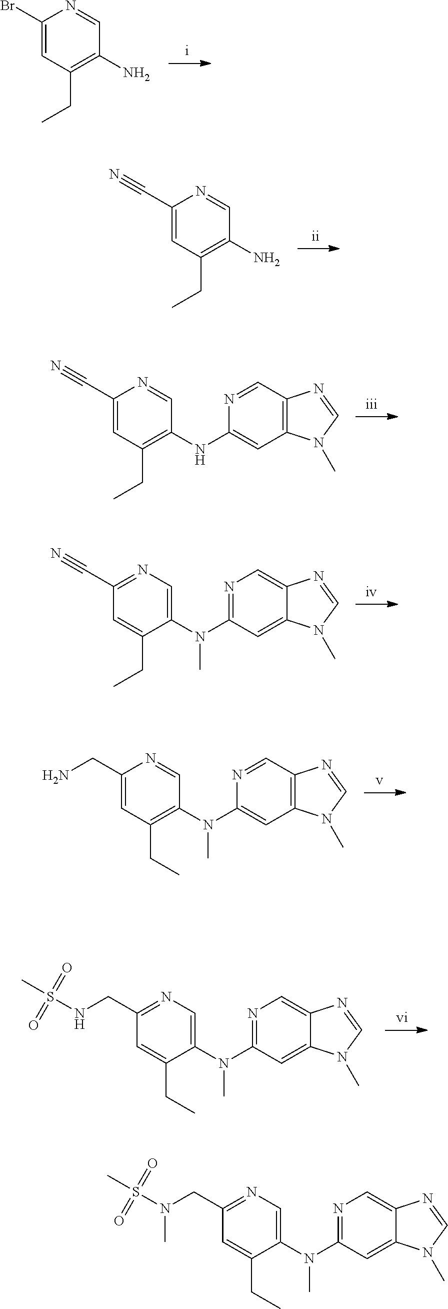

### Step i): 5-Amino-4-ethyl-pyridine-2-carbonitrile

Solution of the bromopyridine (1 equiv, 3 g) and zinc cyanide (0.75 equiv, 1.31 g) in DMF (30 mL) and DMA (10 mL) under inert atmosphere was stirred at 90° C. After 15 min tetrakis(triphenylphosphine) palladium(0) (0.1 equiv, 1.72 g) was added and reaction mixture was stirred at 90° C. for 3 h. The resulting mixture was diluted with water (300 mL) and extracted with EtOAc (5×50 mL). The residue was purified by silica chromatography (EtOAc/cyclohexane; 0:100 to 30:70) to give the desired compound.

### Step ii): 4-Ethyl-5-(1-methyl-1H-imidazo[4,5-c]pyridin-6-ylamino)-pyridine-2-carbonitrile

A mixture of the aniline (1 equiv, 615 mg), 6-chloro-1-methyl-1H-imidazo[4,5-c]pyridine (1 equiv, 700 mg), 2-dicyclohexylphosphino-2′,4′,6′-triisopropylbiphenyl (0.1 equiv, 199 mg), tris(dibenzylideneacetone)-dipalladium (0) (0.1 equiv, 383 mg) and Cs₂CO₃ (2 equiv, 2.72 g) in DMF (8 mL) was stirred at 110° C. for 16 h. The residue was purified by silica chromatography (MeOH/EtOAc; 0:100 to 20:80) to give the desired compound.

### Step iii): 4-Ethyl-5-[methyl-(1-methyl-1H-imidazo[4,5-c]pyridin-6-yl)-amino]-pyridine-2-carbonitrile

NaH (1 equiv, 39.5 mg) was added to a solution of the amine (1 equiv, 275 mg) in DMF (5 mL) at 0° C. After 15 min MeI (1 equiv, 62 μL) was added and resulting mixture was stirred at 0° C. for 3 h. The mixture was concentrated, diluted with water (30 mL) and extracted with EtOAc (5×10 mL). The combined organics were evaporated and the residue purified by silica chromatography (MeOH/DCM; 0:100 to 5:95) to give the desired compound.

### Step iv): (6-Aminomethyl-4-ethyl-pyridin-3-yl)-methyl-(1-methyl-1H-imidazo[4,5-c]pyridin-6-yl)-amine

To a solution of the nitrile (1 equiv, 225 mg) in dry MeOH (10 mL) was added NiCl₂.6H₂O (1 equiv, 183.0 mg) and compound TFA (8 equiv, 472 μL). The mixture was cooled to 0° C. and NaBH₄ (8 equiv, 233.0 mg) was added and the solution was stirred for 2 h. The mixture was filtered over Celite and the filtrate was evaporated in vacuo to give crude product. The residue was put on a previously conditioned SCX Column (1 g). The column was washed with MeOH (3×5 mL) and then with 7N NH₃ solution in MeOH (10 mL) to retrieve the desired product.

### Step v): N-{4-Ethyl-5-[methyl-(1-methyl-1H-imidazo[4,5-c]pyridin-6-yl)-amino]-pyridin-2-ylmethyl}-methanesulfonamide

Triethylamine (2 equiv, 45 μL) was added to a solution of the amine (1 equiv, 150 mg) in DCM (1 mL) followed by the addition of MsCl (1 equiv, 20 μL) and the reaction mixture was stirred at room temperature overnight. The solution was concentrated to give the requivuired product that was used in the next step without further purification.

### Step (vi): N-{4-Ethyl-5-[methyl-(1-methyl-1H-imidazo[4,5-c]pyridin-6-yl)-amino]-pyridin-2-ylmethyl}-N-methyl-methane sulfonamide

NaH (1 equiv, 8.7 mg) was added to a solution of the sulfonamide (1 equiv, 60 mg) in DMF (1 mL) at 0° C. After 15 min, MeI (1 equiv, 14 μL) was added and resulting mixture was stirred at 0° C. for 4 h. The mixture was concentrated and the residue purified by silica chromatography (MeOH/DCM; 0:100 to 100:0) to give the desired compound.

### Compound 318: 1-{4-Ethyl-5-[methyl-(1-methyl-1H-imidazo[4,5-c]pyridin-6-yl)-amino]-pyridin-2-yl}-pyrrolidin-2-one

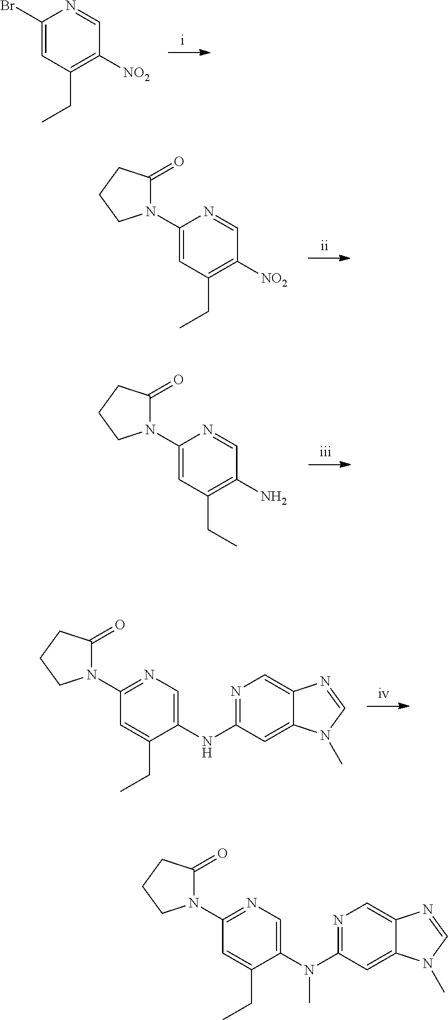 [SMILES: CCc1cc(Br)ncc1[N+](=O)[O-] ; CCc1cc(N2CCCC2=O)ncc1[N+](=O)[O-] ; CCc1cc(N2CCCC2=O)ncc1N ; CCc1cc(N2CCCC2=O)ncc1Nc1cc2c(cn1)ncn2C ; CCc1cc(N2CCCC2=O)ncc1N(C)c1cc2c(cn1)ncn2C]

### Step i): 1-(4-Ethyl-5-nitro-pyridin-2-yl)-pyrrolidin-2-one

A sealed tube containing copper(I) iodide (0.05 equiv, 12 mg), potassium carbonate (2 equiv, 359 mg), pyrrolidinone (2 equiv, 221 mg), (1S,2S)—N,N′-dimethyl-1,2-cyclohexanediamine (0.2 equiv, 37 mg) and 2-bromo-4-ethyl-5-nitro-pyridine (1 equiv, 300 mg) in toluene (10 mL) under nitrogen was heated at 110° C. for 16 h. Reaction mixture was diluted with DCM (20 mL), washed with water (20 mL), dried and concentrated. The residue was purified by silica chromatography (MeOH/DCM; 1:99 to 10:90) to give the desired product.

### Step ii): 1-(5-Amino-4-ethyl-pyridin-2-yl)-pyrrolidin-2-one

A mixture of 1-(4-ethyl-5-nitro-pyridin-2-yl)-pyrrolidin-2-one (1 equiv, 45 mg), zinc (3 equiv, 38 mg) in aq. sat. NH₄Cl solution (5 mL) and EtOH (5 mL) was stirred at 80° C. for 30 min. Water (30 mL) was added and the reaction mixture was extracted with DCM (2×20 mL). The combined organics was washed with brine, dried and concentrated. The residue was purified by silica chromatography (MeOH/DCM; 1:99 to 10:90) to give the desired product.

### Step iii) and iv): 1-{4-Ethyl-5-[methyl-(1-methyl-1H-imidazo[4,5-c]pyridin-6-yl)-amino]-pyridin-2-yl}-pyrrolidin-2-one

Synthesised following the same conditions used for compound 317 (step ii) and iii)).

### Compound 319: N-{5-Ethyl-2-fluoro-4-[methyl-(1-methyl-1H-imidazo[4,5-c]pyridin-6-yl)-amino]-benzyl}-N-methyl-methanesulfonamide

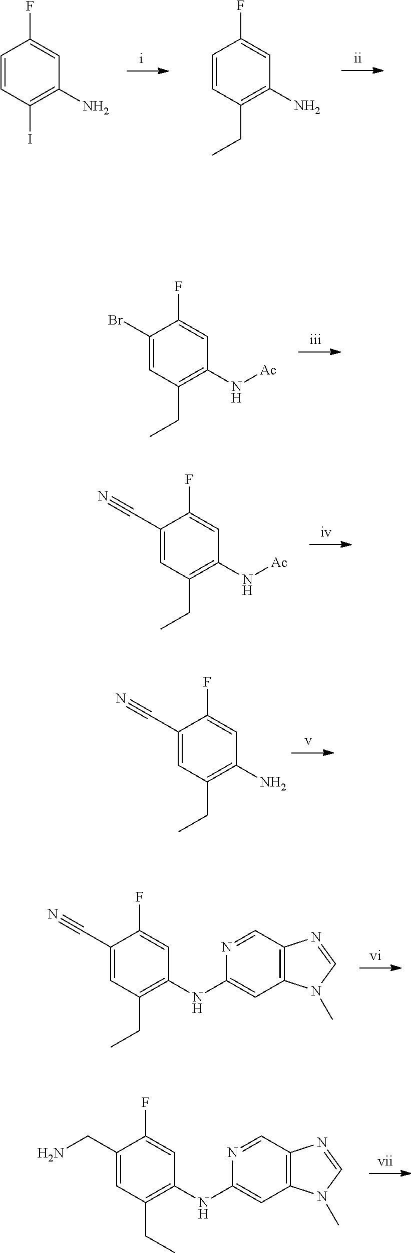

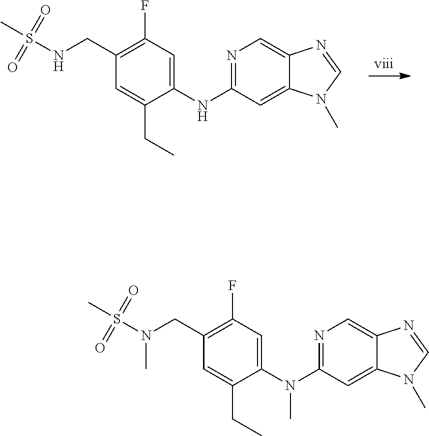 [SMILES: CCc1cc(CNS(C)(=O)=O)c(F)cc1Nc1cc2c(cn1)ncn2C ; CCc1cc(CN(C)S(C)(=O)=O)c(F)cc1N(C)c1cc2c(cn1)ncn2C]

### Step i): 2-Ethyl-5-fluoro-phenylamine

To a mixture of Cs₂CO₃ (6 equiv, 20.6 g) and PdCl₂dppf (0.1 equiv, 0.86 g) in dry DMF (65 mL) under N₂ was added 5-fluoro-2-iodo-phenylamine (1 equiv, 2.5 g) and triethylborane (1M in THF, 1.3 equiv, 13.7 mL). The reaction mixture was heated in a sealed flask at 55° C. for 3 h. The reaction mixture was then filtered over a celite pad, poured into 500 mL of water, extracted with Et₂O (3×150 mL), dried (Na₂SO₄) and concentrated in vacuo. The residue was purified by silica chromatography (EtOAc/cHex; 3:97 to 18:82) to give the desired compound.

### Step ii): N-(4-Bromo-2-ethyl-5-fluoro-phenyl)-acetamide

A solution of the aniline (1 equiv, 1.09 g) and acetic anhydride (1.1 equiv, 0.8 mL) in toluene (10 mL) was heated at reflux for 1 h after what the solution was concentrated in vacuo. The residue was dissolved in glacial acetic acid (7 mL) and bromine (1 equiv, 0.4 mL) was added dropwised and stirred at room temperature for 16 h. The reaction mixture was poured in water (200 mL) and the precipitate formed was filtered, washed with water and dried to afford the desired compound.

### Step iii): N-(4-Cyano-2-ethyl-5-fluoro-phenyl)-acetamide

A suspension of N-(4-bromo-2-ethyl-5-fluoro-phenyl)-acetamide (1 equiv, 1.5 g) and CuCN (1 equiv, 516 mg) in DMF (15 mL) was heated in a sealed tube at 150° C. for 20 h. The reaction mixture was filtered over a Celite pad, the filtrate was poured on water (200 mL) and the resulting precipitate was collected and dried. The residue was redissolved in DCM (50 mL), the suspension filtered and the filtrated concentrated to give the desired product that was used in the next step without further purification.

### Step iv): 4-Amino-5-ethyl-2-fluoro-benzonitrile

A solution of the acetamide (1 equiv, 680 mg) in EtOH (5 mL) and conc. HCl solution (5 mL) was refluxed for 40 min. The resulting mixture was concentrated, dissolved in aq. sat. NaHCO₃ solution (50 mL) and extracted with DCM (3×20 mL). The combined organics were dried, concentrated and purified by silica chromatography (EtOAc/cyclohexane; 8:92 to 50:50) to give the desired compound.

### Step v) and vi): (4-Aminomethyl-2-ethyl-5-fluoro-phenyl)-(1-methyl-1H-imidazo[4,5-c]pyridin-6-yl)-amine

Synthesised following the same conditions used for compound 317 (step ii) and iv))

### Step (vii): N-{5-Ethyl-2-fluoro-4-[methyl-(1-methyl-1H-imidazo[4,5-c]pyridin-6-yl)-amino]-benzyl}-methanesulfon-amide

Pyridine (2 equiv, 6.7 μL) was added to a solution of the benzylamine (1 equiv, 26 mg) in DCM (5 mL) followed by MsCl (1.1 equiv, 6.7 μL) and the solution was stirred at room temperature for 16 h. The reaction mixture was concentrated to give the desired product.

### Step (viii): N-{5-Ethyl-2-fluoro-4-[methyl-(1-methyl-1H-imidazo[4,5-c]pyridin-6-yl)-amino]-benzyl}-N-methyl-methanesulfonamide

NaH (2 equiv, 4.2 mg) was added to a solution of the sulfonamide (1 equiv, 20 mg) in THF (1 mL). After 15 min, MeI (2 equiv, 6.6 μL) was added and resulting mixture was stirred at room temperature for 50 h. The reaction mixture was concentrated to give the desired compound.

### Compound 320: {6-[1-(Difluoro-methanesulfonyl)-azetidin-3-yl]-4-ethyl-pyridin-3-yl}-methyl-(1-methyl-1H-imidazo[4,5-c]pyridin-6-yl)-amine

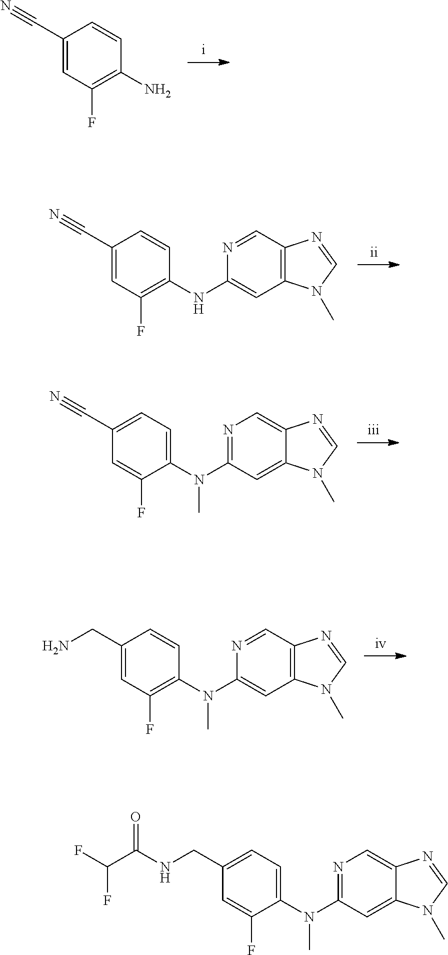 [SMILES: N#Cc1ccc(N)c(F)c1 ; Cn1cnc2cnc(Nc3ccc(C#N)cc3F)cc21 ; CN(c1cc2c(cn1)ncn2C)c1ccc(C#N)cc1F ; CN(c1cc2c(cn1)ncn2C)c1ccc(CN)cc1F ; CN(c1cc2c(cn1)ncn2C)c1ccc(CNC(=O)C(F)F)cc1F]

### Step i) to iii): (4-Aminomethyl-2-fluoro-phenyl)-methyl-(1-methyl-1H-imidazo[4,5-c]pyridin-6-yl)-amine

Synthesised following the same conditions used for compound 317 (step ii) to iv))

### Step iv): {6-[7-(Difluoro-methanesulfonyl)-azetidin-3-yl]-4-ethyl-pyridin-3-yl}-methyl-(1-methyl-1H-imidazo[4,5-c]pyridin-6-yl)-amine

Difluoroacetic anhydride (0.5 equiv, 4.4 μL) was added to a solution of the benzylamine (1 equiv, 20 mg) in THF (1 mL), and the reaction mixture was stirred at room temperature for 30 min. The mixture was concentrated and purified by preparative HPLC to give the desired compound.

### Compound 321: 5-Ethyl-4-[methyl-(1-methyl-1H-imidazo[4,5-c]pyridin-6-yl)-amino]-2-trifluoromethyl-benzonitrile

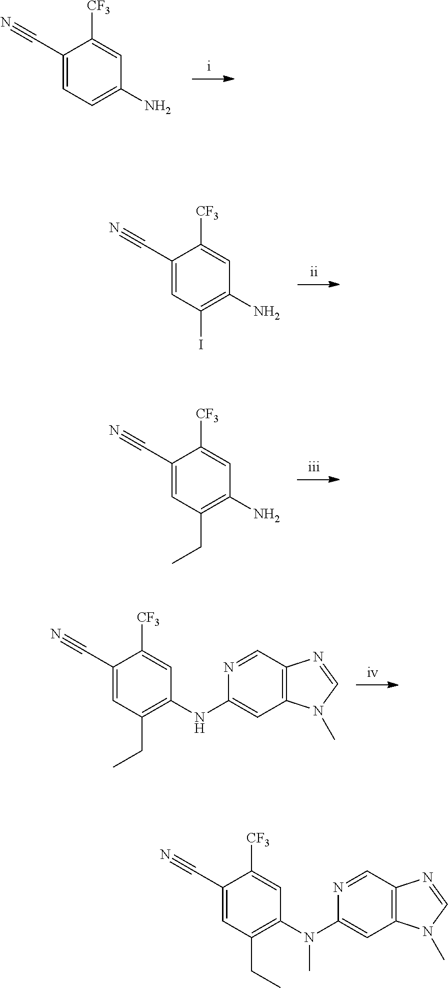 [SMILES: N#Cc1ccc(N)cc1C(F)(F)F ; N#Cc1cc(I)c(N)cc1C(F)(F)F ; CCc1cc(C#N)c(C(F)(F)F)cc1N ; CCc1cc(C#N)c(C(F)(F)F)cc1Nc1cc2c(cn1)ncn2C ; CCc1cc(C#N)c(C(F)(F)F)cc1N(C)c1cc2c(cn1)ncn2C]

### Step i): 4-Amino-5-iodo-2-trifluoromethyl-benzonitrile

To a suspension of 4-amino-2-trifluoromethyl-benzonitrile (1 equiv, 1 g), potassium iodide (0.66 equiv, 0.59 g) and potassium iodate (0.33 equiv, 3.45 g) in MeOH (10 mL) and H₂O (20 mL) was added aq. 1M HCl solution (10 mL) dropwise over 30 min. The resulting mixture was stirred at room temperature for 6 days. The reaction mixture was quenched by the addition of aq. sat. NaHCO₃ solution and extracted with DCM (3×100 mL). The organic layers were combined, washed with aq. sat. Na₂S₂O₃ (2×100 mL) and dried over Na₂SO₄/MgSO₄. After evaporation in vacuo, 4-amino-5-iodo-2-trifluoromethyl-benzonitrile was obtained.

### Step ii): 4-Amino-5-ethyl-2-trifluoromethyl-benzonitrile

To a mixture of Cs₂CO₃ (6 equiv, 9.4 g) and PdCl₂dppf (0.1 equiv, 0.39 g) in dry DMF (30 mL) under N₂ was added 4-amino-5-iodo-2-trifluoromethyl-benzonitrile (1 equiv, 1.5 g) and triethylborane (1M in THF, 1.3 equiv, 6.25 mL). The reaction mixture was heated in a sealed flask at 55° C. for 3 h. The reaction mixture was then filtered over a celite pad, poured into 500 mL of water, extracted with EtOAc (3×150 mL), dried (Na₂SO₄) and concentrated in vacuo. The residue was purified by silica chromatography (EtOAc/cHex; 0:100 to 30:70) to give the desired compound

### Step iii) and iv): 5-Ethyl-4-[methyl-(1-methyl-1H-imidazo[4,5-c]pyridin-6-yl)-amino]-2-trifluoromethyl-benzonitrile

Synthesised following the same conditions used for compound 317 (step ii) and iii))

### Compound 322: 3-Ethyl-5-fluoro-4-[methyl-(1-methyl-M-imidazo[4,5-c]pyridin-6-yl)-amino]-benzonitrile

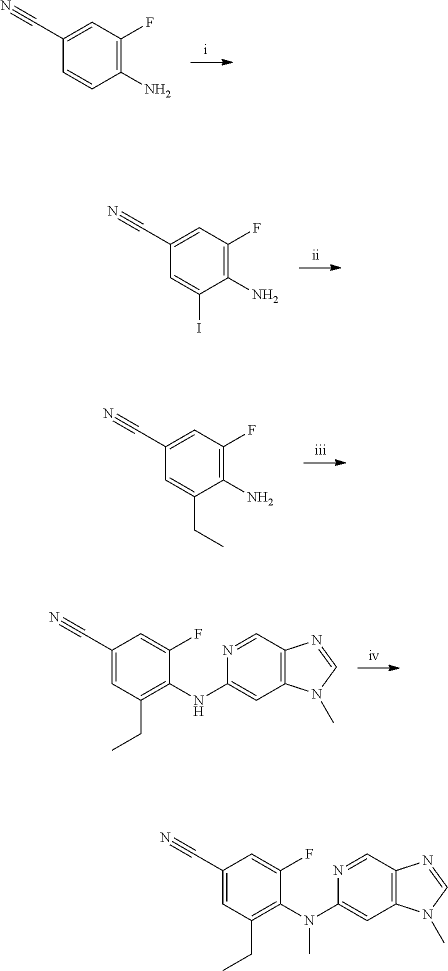 [SMILES: N#Cc1ccc(N)c(F)c1 ; N#Cc1cc(F)c(N)c(I)c1 ; CCc1cc(C#N)cc(F)c1N ; CCc1cc(C#N)cc(F)c1Nc1cc2c(cn1)ncn2C ; CCc1cc(C#N)cc(F)c1N(C)c1cc2c(cn1)ncn2C]

### Step i): 4-Amino-3-fluoro-5-iodo-benzonitrile

To 4-amino-3-fluoro-benzonitrile (1 equiv, 4.06 g) in DCM (100 mL) was added ICl (1.33 equiv, 6.5 g) in DCM (40 mL) and the mixture was stirred at room temperature for 16 h. The reaction was quenched by adding aq. sat. sodium thiosulfate solution and the aqueous solution was extracted with DCM. The combined organics was washed with water, brine, was dried (Na₂SO₄) and concentrated in vacuo to give the desired compound.

### Step ii): 4-Amino-3-ethyl-5-fluoro-benzonitrile

To a mixture of Cs₂CO₃ (6 equiv, 11.5 g) and PdCl₂dppf (0.1 equiv, 0.48 g) in dry DMF (36 mL) under N₂ was added 4-amino-3-fluoro-5-iodo-benzonitrile (1 equiv, 1.56 g) and triethylborane (1M in THF, 1.3 equiv, 7.67 mL). The reaction mixture was heated in a sealed flask at 55° C. for 3 h. The reaction mixture was then filtered over a celite pad, poured into 250 mL of water, extracted with DCM (3×60 mL), dried (Na₂SO₄) and concentrated in vacuo. The residue was purified by silica chromatography (EtOAc/cHex; 0:100 to 25:75) to give the desired compound.

### Step iii) and iv): 3-Ethyl-5-fluoro-4-[methyl-(1-methyl-1H-imidazo[4,5-c]pyridin-6-yl)-amino]-benzonitrile

Synthesised following the same conditions used for compound 317 (step ii) and iii)).

### Compound 323: N-{3-Ethyl-5-fluoro-4-[methyl-(1-methyl-1H-imidazo[4,5-c]pyridin-6-yl)-amino]-benzyl}-methanesulfonamide

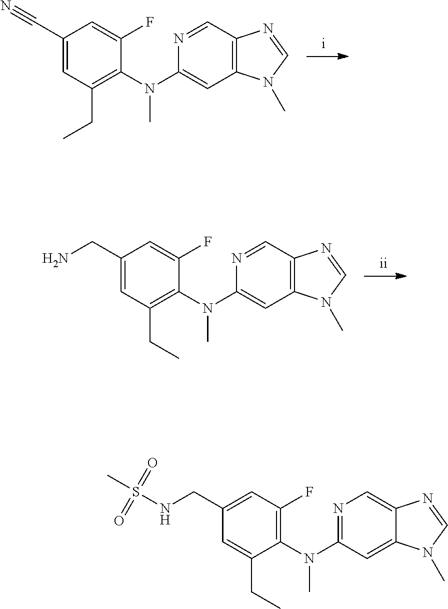 [SMILES: CCc1cc(C#N)cc(F)c1N(C)c1cc2c(cn1)ncn2C ; CCc1cc(CN)cc(F)c1N(C)c1cc2c(cn1)ncn2C ; CCc1cc(CNS(C)(=O)=O)cc(F)c1N(C)c1cc2c(cn1)ncn2C]

Synthesised following the same conditions used for compound 319 (step vi) and vii)).

### Compound 324: N-{3-Ethyl-5-fluoro-4-[methyl-(1-methyl-1H-imidazo[4,5-c]pyridin-6-yl)-amino]-benzyl}-C,C-difluoro-methanesulfonamide

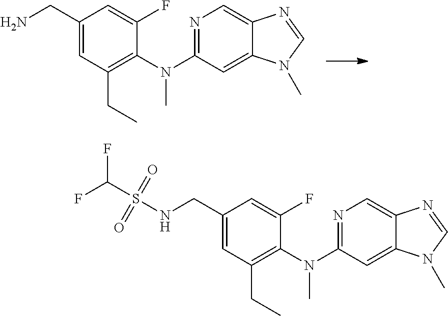

Dry pyridine (3 equiv 35 μL) was added to a solution of the benzylamine (1 equiv, 45 mg) in dry DCM (3 mL) at 0° C., followed by difluoro-methanesulfonyl chloride (1 equiv, 13 μL). The reaction mixture was then stirred at room temperature for 2 h after what it was diluted with water (5 mL) and extracted with DCM (3×10 mL). Organic layers are combined, passed through phase separator and evaporated under reduced pressure. The residue was purified by silica chromatography (MeOH/EtOAc; 0:100 to 20:80) to give the desired compound.

### Compound 325: N-{5-Ethyl-2-fluoro-4-[methyl-(1-methyl-1H-imidazo[4,5-c]pyridin-6-yl)-amino]-benzyl}-methanesulfonamide

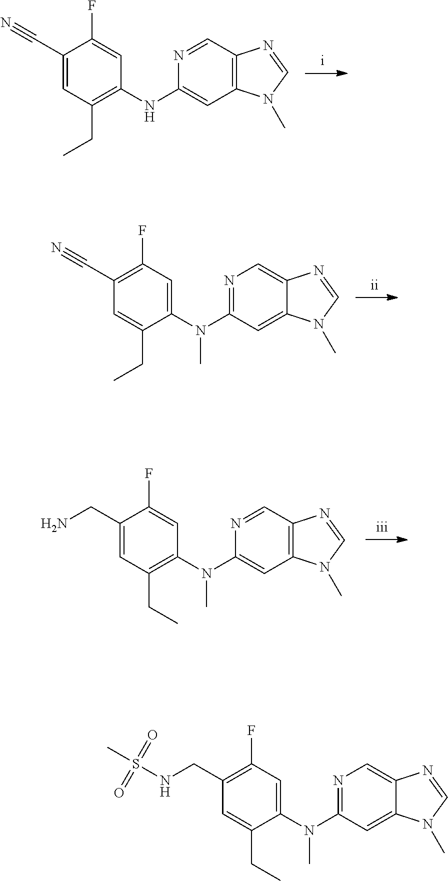 [SMILES: CCc1cc(C#N)c(F)cc1Nc1cc2c(cn1)ncn2C ; CCc1cc(C#N)c(F)cc1N(C)c1cc2c(cn1)ncn2C ; CCc1cc(CN)c(F)cc1N(C)c1cc2c(cn1)ncn2C ; CCc1cc(CNS(C)(=O)=O)c(F)cc1N(C)c1cc2c(cn1)ncn2C]

Synthesised following the same conditions used for compound 319 (step vi) and vii)) and compound 317 (step iii)).

### Compound 326: N-{5-Ethyl-2-fluoro-4-[methyl-(1-methyl-M-imidazo[4,5-c]pyridin-6-yl)-amino]-benzyl}-2,2-difluoro-acetamide

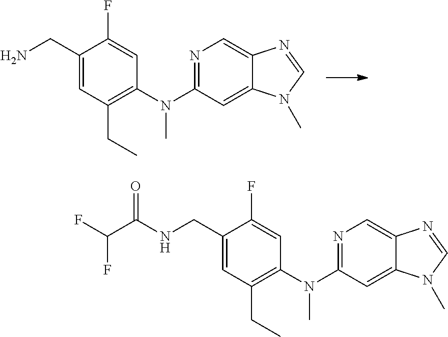

Difluoroacetic anhydride (1 equiv, 9.5 μL) was added to a solution of (4-Aminomethyl-2-ethyl-5-fluoro-phenyl)-methyl-(1-methyl-1H-imidazo[4,5-c]pyridin-6-yl)-amine (1 equiv, 24 mg) in dry DCM (3 mL) at 0° C. The reaction mixture was then stirred at room temperature for 2 h after what it was diluted with water (5 mL) and extracted with DCM (3×10 mL). The organic layers were combined, passed through phase separator and evaporated under reduced pressure. The residue was purified by silica chromatography (MeOH/EtOAc; 0:100 to 20:80) to give the desired compound.

### Compound 327: N-{5-Ethyl-2-fluoro-4-[methyl-(1-methyl-M-imidazo[4,5-c]pyridin-6-yl)-amino]-benzyl}-C,C-difluoro-methanesulfonamide

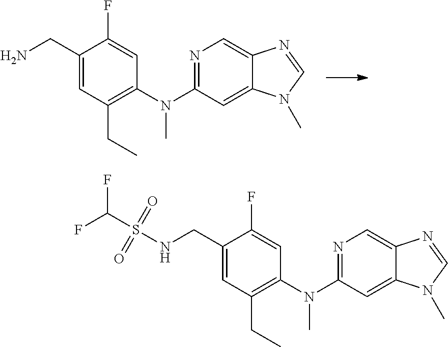

Dry pyridine (3 equiv 29 μL) was added to a solution of the benzylamine (1 equiv, 38 mg) in dry DCM (3 mL) at 0° C., followed by difluoro-methanesulfonyl chloride (1 equiv, 11 μL). The reaction mixture was then stirred at room temperature for 2 h after what it was diluted with water (5 mL) and extracted with DCM (3×10 mL). Organic layers were combined, passed through phase separator and evaporated under reduced pressure. The residue was purified by silica chromatography (MeOH/EtOAc; 0:100 to 20:80) to give the desired compound.

### Compound 328: [4-Ethyl-6-(1-methanesulfonyl-azetidin-3-yl)-pyridin-3-yl]-methyl-(1-methyl-1H-imidazo[4,5-c]pyridin-6-yl)-amine

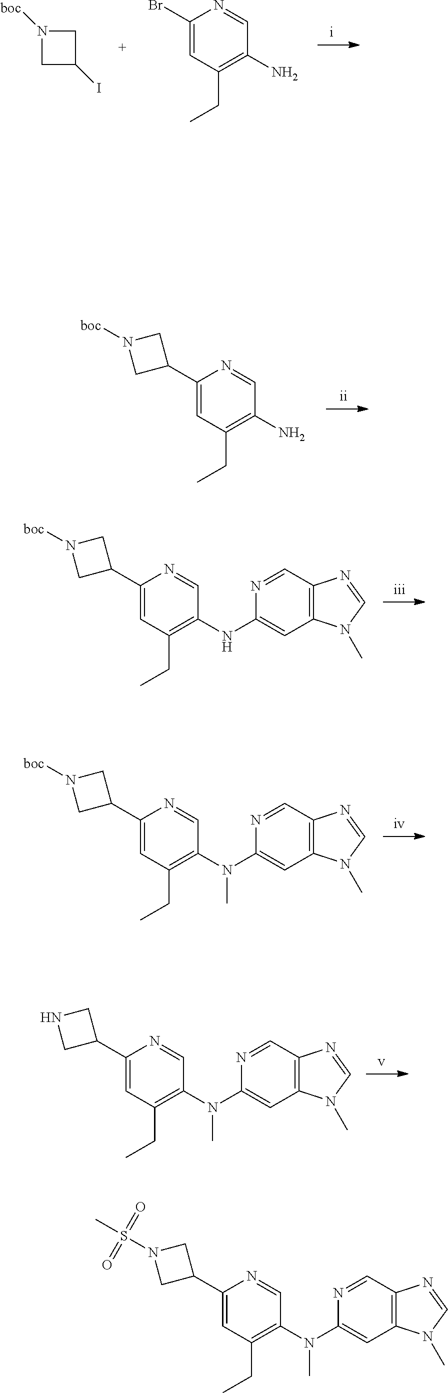

### Step i): 3-(5-Amino-4-ethyl-pyridin-2-yl)-azetidine-1-carboxylic acid tert-butyl ester

N-Boc-azetidine-iodide (3 equiv, 42 g) was dissolved in DMA (40 mL) and heated at 65° C. Under atmosphere of nitrogen, Rieke zinc (3.1 equiv, 200 mL, 9.9 g) was added dropwise over 10 min and stirred for 20 min at 65° C. under nitrogen. In another flask 6-bromo-4-ethyl-pyridin-3-ylamine (1 equiv, 10 g), copper(I) iodide (0.01 equiv, 100 mg) and PdCl₂dppf (0.03 equiv, 1.13 g) were dissolved in DMA (40 mL) and heated at 85° C. under atmosphere of nitrogen. The reaction mixture from the first flask was added via a cannula to the second flask over 10 min. The resulting reaction mixture was stirred at 85° C. for 5 min. The reaction was then quenched with aq. sat. NH₄Cl solution and extracted with EtOAc (3×200 mL). The combined organics was dried and concentrated under reduced pressure. The residue was purified by column chromatography (EtOAc/petroleum ether 40-60; 0:100 to 100:0) to give the desired product.

### Step (ii): 3-[4-Ethyl-5-(1-methyl-1H-imidazo[4,5-c]pyridin-6-ylamino)-pyridin-2-yl]-azetidine-1-carboxylic acid tert-butyl ester

A mixture of 6-chloro-1-methyl-1H-imidazo[4,5-c]pyridine (1.1 equiv, 1.56 g), 3-(5-Amino-4-ethyl-pyridin-2-yl)-azetidine-1-carboxylic acid tert-butyl ester (1.0 equiv, 2.35 g) and Cs₂CO₃ (3.0 equiv, 8.29 g) in dry 1,4-dioxane (10 mL) was purged with nitrogen. Afterwards, Pd₂ dba₃ (0.1 equiv, 0.78 g) and BINAP (0.2 equiv, 1.06 g) were added, the reaction mixture was purged again with nitrogen and was stirred at 110° C. After 18 h, water was added and the mixture was extracted with EtOAc (3×). The organics were combined, dried and evaporated under reduced pressure to afford the desired product.

### Step (iii): 3-{4-Ethyl-5-[methyl-(1-methyl-1H-imidazo[4,5-c]pyridin-6-yl)-amino]-pyridin-2-yl}-azetidine-1-carboxylic acid tert-butyl ester

Sodium hydride (60% dispersion in mineral oil, 2.6 equiv, 1.76 g) was added to a solution of 3-[4-ethyl-5-(1-methyl-1H-imidazo[4,5-c]pyridin-6-ylamino)-pyridin-2-yl]-azetidine-1-carboxylic acid tert-butyl ester (1.0 equiv, 6.92 g) was dissolved in dry THF (100 mL) at 0° C. After 30 min, iodomethane (2.0 equiv, 2.1 mL) was added and the reaction was stirred at room temperature for 16 h. The reaction was quenched with cold water; the compound was extracted with DCM, dried and concentrated under vacuum. The compound was purified by silica flash chromatography (Interchim, Puriflash 450) (EtOAc/petroleum ether 40-60; 5:95 to 100:0) to afford the pure product.

### Step (iv): (6-Azetidin-3-yl-4-ethyl-pyridin-3-yl)-methyl-(1-methyl-1H-imidazo[4,5-c]pyridin-6-yl)-amine

3-{4-Ethyl-5-[methyl-(1-methyl-1H-imidazo[4,5-c]pyridin-6-yl)-amino]-pyridin-2-yl}-azetidine-1-carboxylic acid tert-butyl ester (1.0 equiv, 7.16 g) was added to a mixture TFA/DCM (1:1) (100 mL) and stirred at room temperature for 2 h. The resulting mixture was concentrated under vacuum. The residue was purified by a SCX column: the column was equilibrated with a solution of 5% AcOH in MeOH, the impurities were eluted with MeOH and the compound was eluted with a solution 2N NH₃ in MeOH to afford the pure product.

### Step (v): [4-Ethyl-6-(1-methanesulfonyl-azetidin-3-yl)-pyridin-3-yl]-methyl-(1-methyl-1H-imidazo[4,5-c]pyridin-6-yl)-amine

Triethylamine (1.1 equiv, 3 mL) was added to a solution of [4-Ethyl-6-(1-methanesulfonyl-azetidin-3-yl)-pyridin-3-yl]-methyl-(1-methyl-1H-imidazo[4,5-c]pyridin-6-yl)-amine (1.0 equiv, 6.3 g) in DCM (290 mL) at 0° C. A solution of methylsulfonyl chloride 1M in DCM (1.1 equiv, 21.5 mL) was added dropwise over 30 min and the resulting mixture was stirred at room temperature for 15 min. The mixture was diluted with DCM and washed with cold water. The organic layer was dried over Na₂SO₄, filtered and concentrated under vacuum. The residue was purified by silica flash chromatography (Interchim, Puriflash 450) (MeOH/EtOAc; 3:97 to 10:90) to afford the desired compound.

### Compound 329: {6-[1-(Difluoro-methanesulfonyl)-azetidin-3-yl]-4-ethyl-pyridin-3-yl}-methyl-(1-methyl-1H-imidazo[4,5-c]pyridin-6-yl)-amine

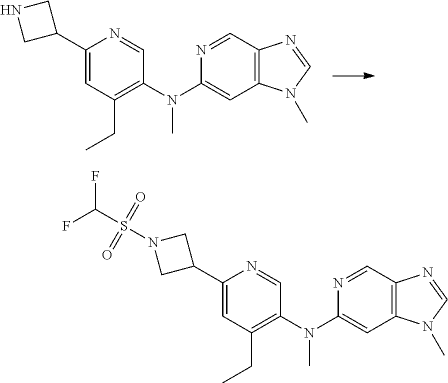

Dry pyridine (5 equiv 19 μL) was added to a solution of (6-azetidin-3-yl-4-ethyl-pyridin-3-yl)-methyl-(1-methyl-1H-imidazo[4,5-c]pyridin-6-yl)-amine (1 equiv, 15 mg) in dry DCM (2 mL). The reaction mixture was stirred at room temperature for 15 min followed by the addition dropwise of difluoro-methanesulfonyl chloride (1 equiv, 19 μL) at 0° C. After 15 min, the reaction mixture was diluted with water (5 mL) and extracted with DCM (3×10 mL). Organic layers are combined, passed through phase separator and evaporated under reduced pressure. The residue was purified by silica chromatography (MeOH/EtOAc; 0:100 to 20:80) to give the desired compound.

### Compound 330: {4-Ethyl-6-[1-(propane-2-sulfonyl)-azetidin-3-yl]-pyridin-3-yl}-methyl-(1-methyl-1H-imidazo[4,5-c]pyridin-6-yl)-amine

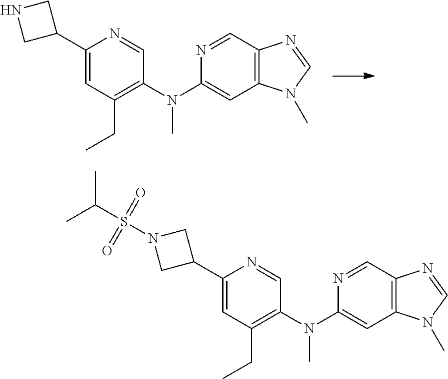

Dry pyridine (5 equiv 33 μL) was added to a solution of (6-azetidin-3-yl-4-ethyl-pyridin-3-yl)-methyl-(1-methyl-1H-imidazo[4,5-c]pyridin-6-yl)-amine (1 equiv, 35 mg) in dry DCM (3 mL) at 0° C. followed by the addition of isopropylsulfonyl chloride (1.2 equiv, 10 μL). The reaction mixture was then stirred at room temperature after what it was diluted with water (5 mL) and extracted with DCM (3×10 mL). Organic layers were combined, passed through phase separator and evaporated under reduced pressure. The residue was purified by silica chromatography (MeOH/EtOAc; 0:100 to 20:80) to give the desired compound.

### Compound 331: (3-{4-Ethyl-5-[methyl-(1-methyl-1H-imidazo[4,5-c]pyridin-6-yl)-amino]-pyridin-2-yl}-azetidin-1-yl)-acetonitrile

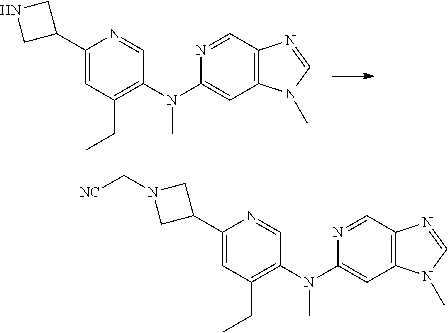

Sodium hydride (1 equiv, 5.5 mg) was added to a solution of the azetidine (1 equiv, 50 mg) in THF (2 mL) followed by the addition of MeI (1 equiv, 9.6 μL). After 2 h the reaction mixture was diluted with water and extracted with EtOAc (3×). The organics were concentrated to give the desired compound. The combined organics was dried and concentrated. The residue was purified by silica chromatography (MeOH/DCM; 0:100 to 20:80) to give the desired compound.

### Compound 332: 1-(3-{4-Ethyl-5-[methyl-(1-methyl-1H-imidazo[4,5-c]pyridin-6-yl)-amino]-pyridin-2-yl}-azetidin-1-yl)-ethanone

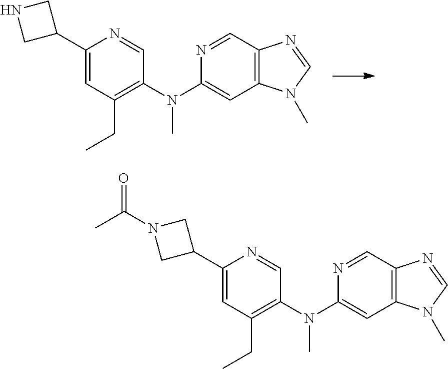

Dry pyridine (2 equiv 23 μL) was added to a solution of (6-azetidin-3-yl-4-ethyl-pyridin-3-yl)-methyl-(1-methyl-1H-imidazo[4,5-c]pyridin-6-yl)-amine (1 equiv, 50 mg) in dry DCM (3 mL) at 0° C. followed by the addition of acetylchloride (1 equiv, 10 μL). The reaction mixture was then stirred at room temperature for 2 h after what it was diluted with water (5 mL) and extracted with DCM (3×10 mL). The organic layers were combined, passed through phase separator and evaporated under reduced pressure. The residue was purified by silica chromatography (MeOH/DCM; 0:100 to 20:80) to give the desired compound.

### Compound 333: 1-(3-{4-Ethyl-5-[methyl-(1-methyl-1H-imidazo[4,5-c]pyridin-6-yl)-amino]-pyridin-2-yl}-azetidin-1-yl)-2,2-difluoro-ethanone

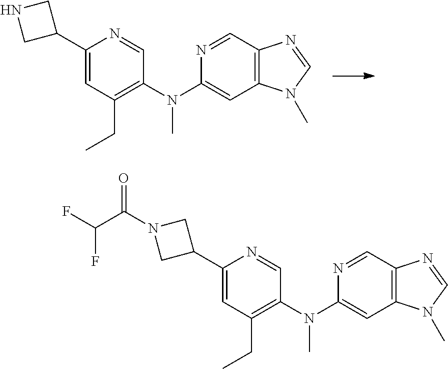

Difluoroacetic anhydride (1 equiv, 17 μL) was added to a solution of (6-azetidin-3-yl-4-ethyl-pyridin-3-yl)-methyl-(1-methyl-1H-imidazo[4,5-c]pyridin-6-yl)-amine (1 equiv, 44 mg) in dry DCM (3 mL) at 0° C. The reaction mixture was then stirred at room temperature for 2 h after what it was diluted with water (5 mL) and extracted with DCM (3×10 mL). The organic layers were combined, passed through phase separator and evaporated under reduced pressure. The residue was purified by silica chromatography (MeOH/EtOAc; 0:100 to 20:80) to give the desired compound.

### Compound 334: 1-(3-{4-Ethyl-5-[methyl-(1-methyl-1H-imidazo[4,5-c]pyridin-6-yl)-amino]-pyridin-2-yl}-azetidin-1-yl)-2,2,2-trifluoro-ethanone

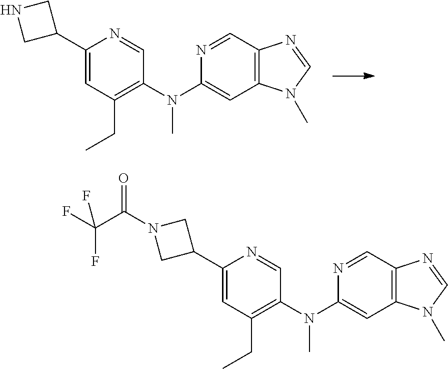

TFA (1 equiv 11 μL) was added to a solution of (6-azetidin-3-yl-4-ethyl-pyridin-3-yl)-methyl-(1-methyl-1H-imidazo[4,5-c]pyridin-6-yl)-amine (1 equiv, 46 mg) in dry DCM (3 mL). After 3 h, the reaction mixture was diluted with water (5 mL) and extracted with DCM (3×10 mL). The organic layers were combined, passed through phase separator and evaporated under reduced pressure. The residue was purified by silica chromatography (MeOH/EtOAc; 0:100 to 20:80) to give the desired compound.

### Compound 335: 3-(3-{4-Ethyl-5-[methyl-(1-methyl-1H-imidazo[4,5-c]pyridin-6-yl)-amino]-pyridin-2-yl}-azetidin-1-yl)-3-oxo-propionitrile

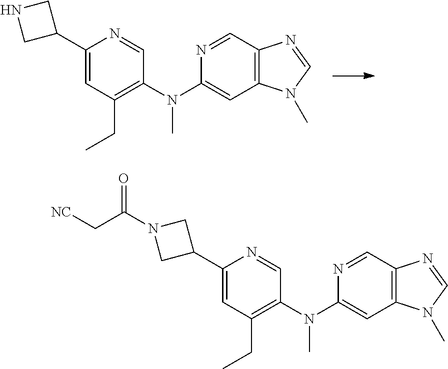

Cyanoacetic acid (1.5 equiv, 31 mg) and EDC (1.5 equiv, 71 mg) were added to a solution of the azetidine (1.0 equiv, 80 mg) in DCM (3 mL) followed by the addition of DIPEA (1.5 equiv, 64 μL). The reaction was stirred at room temperature until full conversion, the solution was then diluted with DCM, washed with aq. sat. NaHCO₃, dried and concentrated. The residue was triturated (Et₂O, i-Pr₂O) to give the desired compound.

### Compound 336: 1-(3-{4-Ethyl-5-[methyl-(1-methyl-1H-imidazo[4,5-c]pyridin-6-yl)-amino]-pyridin-2-yl}-azetidin-1-yl)-propan-1-one

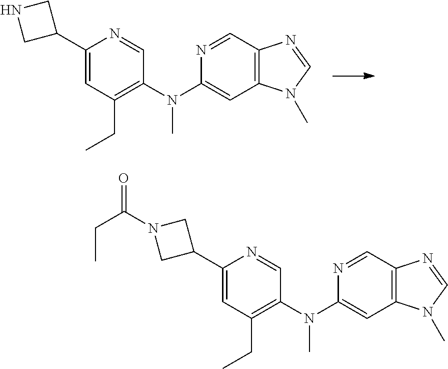

Propionic acid (1.5 equiv, 69 mg) and EDC (1.5 equiv, 179 mg) were added to a solution of the azetidine (1.0 equiv, 200 mg) in DCM (6 mL) followed by the addition of DIPEA (1.5 equiv, 162 μL). The reaction was stirred at room temperature until full conversion after what, the solution was diluted with DCM, washed with aq. sat. NaHCO₃, dried and concentrated. The residue was purified by silica chromatography (MeOH/EtOAc; 0:100 to 10:90) to give the desired compound.

### Compound 337: (3-{4-Ethyl-5-[methyl-(1-methyl-1H-imidazo[4,5-c]pyridin-6-yl)-amino]-pyridin-2-yl}-azetidin-1-yl)-(tetrahydro-pyran-4-yl)-methanone

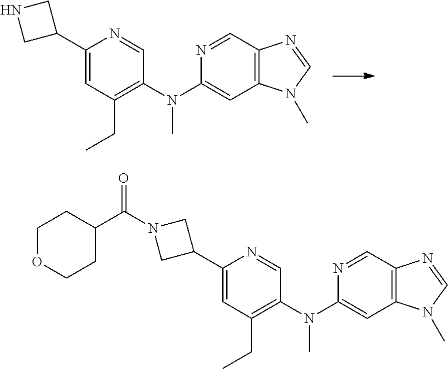

Tetrahydro-pyran-4-carboxylic acid (1.5 equiv, 121 mg) and EDC (1.5 equiv, 179 mg) were added to a solution of the azetidine (1.0 equiv, 200 mg) in DCM (6 mL) followed by the addition of DIPEA (1.5 equiv, 162 μL). The reaction was stirred at room temperature until full conversion, the solution was then diluted with DCM, washed with aq. sat. NaHCO₃ solution, dried and concentrated. The residue was triturated (Et₂O, petrol ether) to give the desired compound.

### Compound 338: 1-(3-{4-Ethyl-5-[methyl-(1-methyl-1H-imidazo[4,5-c]pyridin-6-yl)-amino]-pyridin-2-yl}-azetidin-1-yl)-4,4,4-trifluoro-butan-1-one

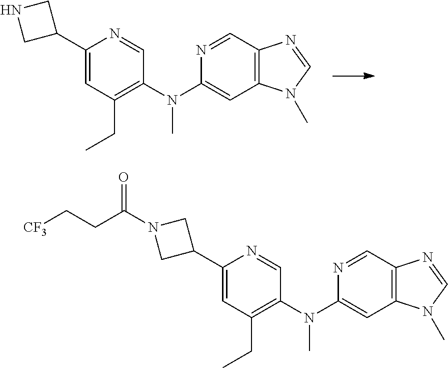

4,4,4-Trifluoro-butyric acid (1.5 equiv, 132 mg) and EDC (1.5 equiv, 179 mg) were added to a solution of the azetidine (1.0 equiv, 200 mg) in DCM (6 mL) followed by the addition of DIPEA (1.5 equiv, 162 μL). The reaction was stirred at room temperature until full conversion, the solution was then diluted with DCM, washed with aq. sat. NaHCO₃ solution, dried and concentrated. The residue was purified by silica chromatography (MeOH/EtOAc; 0:100 to 10:90) to give the desired compound.

### Compound 339: 3-{4-Ethyl-5-[methyl-(1-methyl-1H-imidazo[4,5-c]pyridin-6-yl)-amino]-pyridin-2-yl}-azetidine-1-carboxylic acid methyl ester

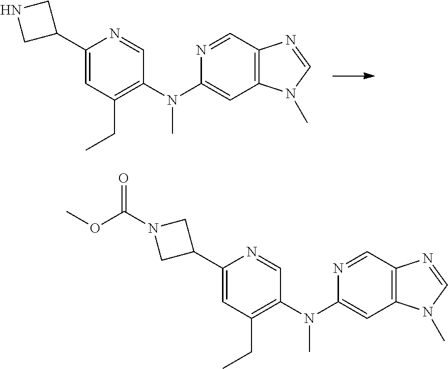

A solution of methyl chloroformate (1 equiv, 24 μL) in DCM (2 mL) was added dropwised to a previously cooled solution (0° C.) of the azetidine (1 equiv, 100 mg) and DIPEA (1 equiv, 54 μL) in DCM (4 mL). The reaction was stirred at 0° C. until consumption of the azetidine. The mixture was diluted with DCM, quenched with water. The layer were separated, the organic washed with aq. sat. NaHCO₃, dried and concentrated. Due to the presence of an impurity, the residue was dissolved in HCOOH (4 mL) and the resulting mixture was stirred at 85° C. The mixture was then concentrated, diluted in DCM, washed with aq. sat. NaHCO₃ solution, dried and concentrated. The residue was triturated (Et₂O, i-Pr₂O, petroleum ether) to give the desired compound.

### Compound 340: (3,3-Difluoro-cyclobutyl)-(3-{4-ethyl-5-[methyl-(1-methyl-1H-imidazo[4,5-c]pyridin-6-yl)-amino]-pyridin-2-yl}-azetidin-1-A-methanone

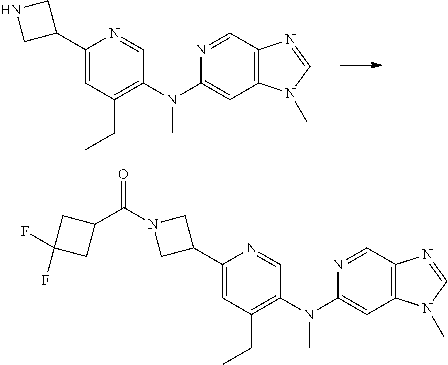

A solution of 3,3-difluoro-cyclobutanecarboxylic acid (1 equiv, 84 mg) and EDC (1 equiv, 119 mg) in DCM (2 mL) at 0° C. was added dropwised to a solution of the azetidine (1 equiv, 200 mg) in DCM (4 mL) at 0° C. The reaction was stirred until full consumption of the azetidine, the solution then was diluted with DCM, washed with aq. sat. NaHCO₃ solution, dried and concentrated. The residue was purified by silica chromatography (MeOH/EtOAc; 0:100 to 5:95) to give the desired compound.

### Compound 341: {4-Ethyl-6-[1-(3,3,3-trifluoro-propyl)-azetidin-3-yl]-pyridin-3-yl}-methyl-(1-methyl-1H-imidazo[4,5-c]pyridin-6-yl)-amine

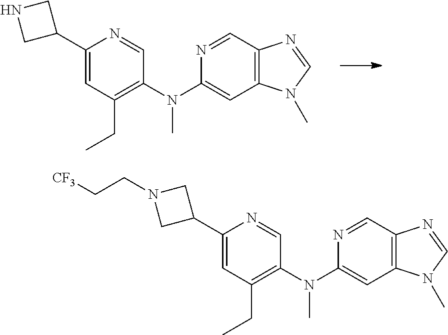

A mixture of the azetidine (1 equiv, 20 mg), 3-bromo-1,1,1-trifluoro-propane (1.9 equiv, 20 mg) and K₂CO₃ (2.3 equiv, 20 mg) was stirred in MeCN (0.5 mL) at 70° C. After completion, the solution was diluted with DCM, washed with aq. sat. NaHCO₃ solution, dried and concentrated. The residue was purified by preparative HPLC to give the desired compound.

### Compound 342: {4-Ethyl-6-[1-(2,2,2-trifluoro-ethyl)-azetidin-3-yl]-pyridin-3-yl}-methyl-(1-methyl-1H-imidazo[4,5-c]pyridin-6-yl)-amine

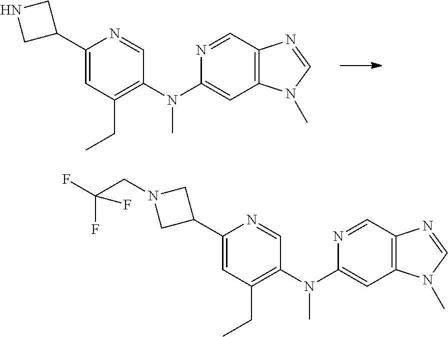

A mixture of the azetidine (1 equiv, 20 mg), 1,1,1-trifluoro-2-iodo-ethane (4.6 equiv, 60 mg) and K₂CO₃ (2.3 equiv, 20 mg) was stirred in MeCN (0.5 mL) at 70° C. After completion, the solution was diluted with DCM, washed with aq. sat. NaHCO₃ solution, dried and concentrated. The residue was purified by preparative HPLC to give the desired compound.

### Compound 343: N-{4-Ethyl-5-[methyl-(1-methyl-1H-imidazo[4,5-c]pyridin-6-yl)-amino]-pyridin-2-ylmethyl}-C,C-difluoro-methane sulfonamide

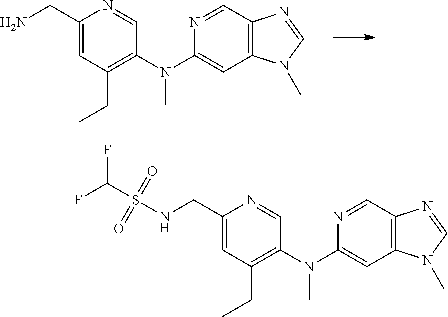

Dry pyridine (3 equiv 45 μL) was added to a solution of the benzylamine (1 equiv, 50 mg) in dry DCM (1 mL) followed by difluoro-methanesulfonyl chloride (1 equiv, 15 μL) and the reaction was stirred overnight at room temperature. The reaction mixture was concentrated and the residue purified by preparative HPLC to yield the desired compound.

### Compound 344: N-Cyanomethyl-N-{4-ethyl-5-[methyl-(1-methyl-1H-imidazo[4,5-c]pyridin-6-yl)-amino]-pyridin-2-ylmethyl}-methanesulfonamide

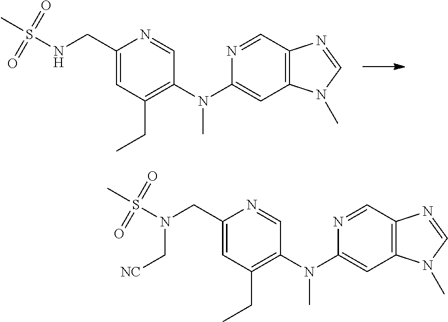

Sodium hydride (2 equiv, 17 mg) was added to a solution of the sulfonamide (1 equiv, 60 mg) in DMF (1 mL) at 0° C. After 15 min, iodoacetonitrile (1 equiv, 32 μL) was added and the solution was stirred at room temperature for 6 h. The reaction mixture was concentrated in vacuo and purified by preparative HPLC to give the desired compound.

### Compound 345: (E)-3-{4-[Methyl-(1-methyl-1H-imidazo[4,5-c]pyridin-6-yl)-amino]-phenyl}-acrylonitrile

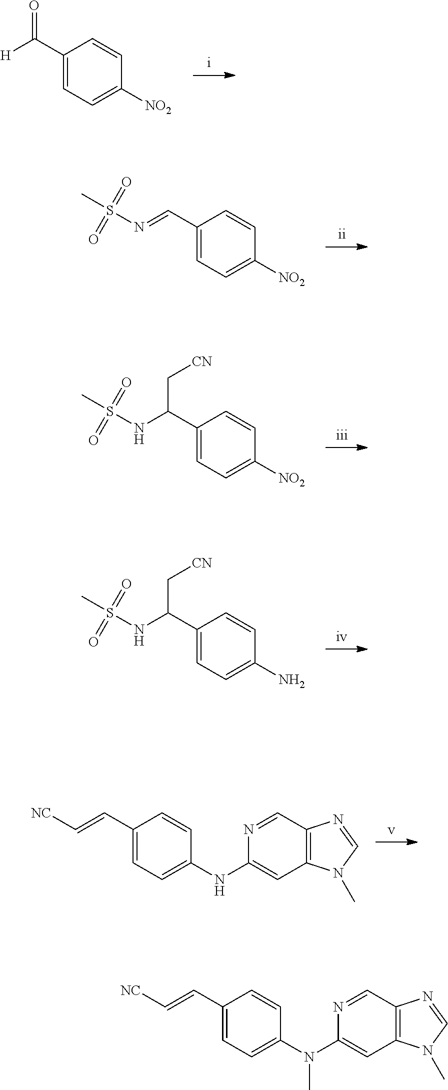 [SMILES: O=Cc1ccc([N+](=O)[O-])cc1 ; CS(=O)(=O)/N=C/c1ccc([N+](=O)[O-])cc1 ; CS(=O)(=O)NC(CC#N)c1ccc([N+](=O)[O-])cc1 ; CS(=O)(=O)NC(CC#N)c1ccc(N)cc1 ; Cn1cnc2cnc(Nc3ccc(/C=C/C#N)cc3)cc21 ; CN(c1ccc(/C=C/C#N)cc1)c1cc2c(cn1)ncn2C]

### Step (i): N-[7-(4-Nitro-phenyl)-meth-(E)-ylidene]-methanesulfonamide

A mixture of 4-nitro-benzaldehyde (1 equiv. 10 g), methanesulfonamide equiv, 6.1 g) and tetraethoxysilane (1.05 equiv, 15.5 mL) was heated at 120° C. for 16 h. The reaction mixture was cooled to room temperature and crystallized with EtOAc/petrol ether. The resulting solid was collected by filtration and dried the desired compound.

### Step (ii): N-[2-Cyano-1-(4-nitro-phenyl)-ethyl]-methanesulfonamide

A mixture of N-(4-nitro-benzylidene)-methanesulfonamide (1 equiv, 2 g), trimethylsilylacetonitrile (1.4 equiv, 1.7 mL) and lithium acetate (0.1 equiv, 0.06 g) was stirred at room temperature for 16 h. Aq. sat. NH₄Cl solution was added and the product extracted with EtOAc (5×). The combined organics was concentrated and purified by silica chromatography (EtOAc/cyclohexane; 0:100 to 100:0) to give the desired product.

### Step (iii): N-[1-(4-Amino-phenyl)-2-cyano-ethyl]-methanesulfonamide

Iron powder (3 equiv, 124 mg) was added to a stirred suspension of ammonium chloride (3 equiv, 119 mg) and the nitrobenzene (1 equiv, 200 mg) in a mixture of acetone (5 mL) and water (1 mL) and the resulting mixture was stirred at 65° C. for 4 h. The mixture was filtered through Celite and washed with EtOAc (5×2 mL). The filtrate was washed with aq. sat. NaHCO₃ (15 mL) and the organic layer was dried and concentrated. The residue was added on a previously conditioned SCX Column (1 g), washed with MeOH (3×5 mL) and then with 7N NH₃ solution in MeOH (10 mL) to retrieve the desired compound.

### Step (iv) and (v): (E)-3-{4-[Methyl-(1-methyl-1H-imidazo[4,5-c]pyridin-6-yl)-amino]-phenyl}-acrylonitrile

Synthesised following the same conditions used for compound 317 (step (ii) and (iii))

### Compound 346: 1-(3-{3-Ethyl-4-[methyl-(1-methyl-1H-imidazo[4,5-c]pyridin-6-yl)-amino]-phenyl)-azetidin-1-yl)-2,2-difluoro-ethanone

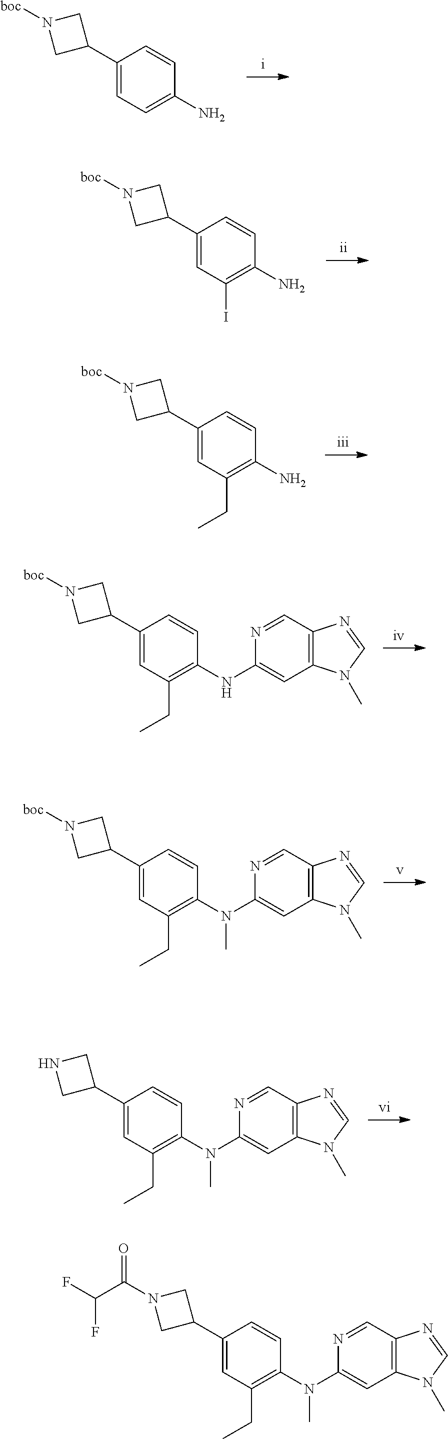

### Step (i): 3-(4-Amino-3-iodo-phenyl}-azetidine-1-carboxylic acid tert-butyl ester

Iodine (1 equiv, 0.1 g) was added to a solution of 3-(4-amino-phenyl)-azetidine-1-carboxylic acid tert-butyl ester (1 equiv, 0.1 g) and Ag₂SO₄ (1 equiv, 0.12 EtOH (2 ml.) and the reaction mixture was stirred at room temperature for 30 min after what the mixture was filtered and concentrated. The residue was diluted in DCM, washed with aq. sat. Na₂SO₃ solution (3×), dried and concentrated. Purification by silica chromatography (EtOAc/petrol ether; 1:10 afforded the desired compound.

### Step (ii): 3-(4-Amino-3-ethyl-phenyl)-azetidine-1-carboxylic acid tert-butyl ester

The aniline derivative (1 equiv, 0.7 g) and triethylborane (1 M in THF, 1.3 equiv, 2.4 mL) were added to a suspension of Cs₂CO₃ (6 equiv, 3.7 g) and PdCl₂dppf (0.1 equiv, 152 mg) in dry DMF (3 mL) and the reaction mixture was stirred at 55° C. for 30 min. The reaction mixture was diluted in EtOAc and washed with water (3×), dried and concentrated. The residue was purified by silica chromatography (EtOAc/petrol; 1:4) to give the desired compound.

### Step (iii) and (iv): 3-{3-Ethyl-4-[methyl-(1-methyl-1H-imidazo[4,5-c]pyridin-6-yl)-amino]-phenyl}-azetidine-1-carboxylic acid tert-butyl ester

A mixture of the aniline derivative (1 equiv, 1.63 g), 6-chloro-1-methyl-1H-imidazo[4,5-c]pyridine (1.1 equiv, 1.48 g), BINAP (0.3 equiv, 1 g), Cs₂CO₃ (3 equiv, 5.77 g) and tris(dibenzylideneacetone)dipalladium(0) (0.15 equiv, 0.81 mg) in dry 1,4-dioxane (20 mL), degassed under (N₂), was heated at 110° C. for 4 h. The solution was concentrated; the residue was diluted in DCM, washed with water (3×) and concentrated in vacuo. NaH (60%, 2 equiv, 472 mg) was added to a solution of the residue in THF (20 mL). After 5 min, MeI (2 equiv, 0.74 mL) was added and the solution was stirred at room temperature for 16 h. After concentrated, the residue was diluted with DCM, washed with water (3×), dried and purified by silica chromatography (MeOH/EtOAc; 5:95) to give the desired product.

### Step (v): (4-Azetidin-3-yl-2-ethyl-phenyl)-methyl-(1-methyl-1H-imidazo[4,5-c]pyridin-6-yl)-amine

The Boc-azetidine (1 equiv, 1.49 equiv) was stirred in TEA 10 mL) and DCM (10 for 5 min. The solution was concentrated and purified through a SCX column equivuilibrated with AcOH/1\400H (5:95) and eluted with MeOH and NH₃ (2. Min McOH) to afford the product.

### Step (vi): 1-(3-{3-Ethyl-4-[methyl-(1-methyl-M-imidazo[4,5-c]pyridin-6-yl)-amino]-phenyl}-azetidin-1-yl)-2,2-difluoro-ethanone

Oxalyl chloride (1.8 equiv, 29 μL) was added to difluoroacetic acid (1.5 equiv, 27 mg) in DCM (3 mL) followed by a drop of DMF. After stirring at room temperature for 40 min, pyridine (2 mL) and the azetidine (1 equiv, 100 mg) and the mixture stirred at room temperature for 16 h. The reaction mixture was diluted with DCM, washed with aq. sat. NaHCO₃ solution, dried and concentrated. Purification by silica chromatography (7M NH₃ solution in MeOH/EtOAc; 10:90) afforded the desired compound.

### Compound 347: 1-(3-{3-Ethyl-4-[methyl-(1-methyl-1H-imidazo[4,5-c]pyridin-6-yl)-amino]-phenyl}-azetidin-1-yl)-2,2,2-trifluoro-ethanone

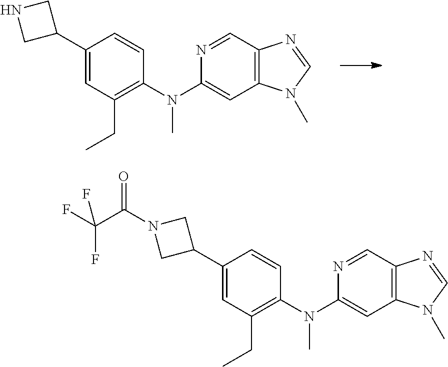

Oxalyl chloride (1.8 equiv, 29 μL) was added to trifluoroacetic acid (1.5 equiv, 32 mg) in DCM (3 mL) followed by a drop of DMF. After stirring at room temperature for 40 min, pyridine (2 mL) and the azetidine (1 equiv, 100 mg) and the mixture stirred at room temperature for 16 h. The reaction mixture was diluted with DCM, washed with aq. sat. NaHCO₃, dried and concentrated.

Purification by silica chromatography (7M NH₃ solution in MeOH/EtOAc; 10:90) afforded the desired compound.

### Compound 348: 1-(3-{3-Ethyl-4-[methyl-(1-methyl-1H-imidazo[4,5-c]pyridin-6-yl)-amino]-phenyl}-azetidine-1-carbonyl)-cyclopropanecarbonitrile

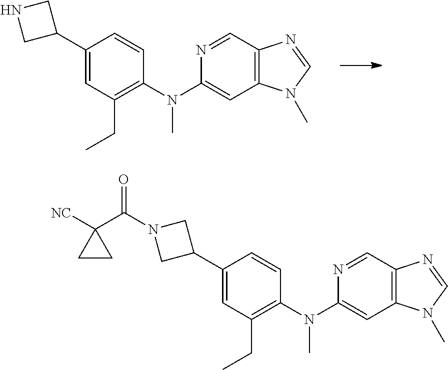 [SMILES: CCc1cc(C2CNC2)ccc1N(C)c1cc2c(cn1)ncn2C ; CCc1cc(C2CN(C(=O)C3(C#N)CC3)C2)ccc1N(C)c1cc2c(cn1)ncn2C]

Oxalyl chloride (1.8 equiv, 29 μL) was added to 1-cyano-cyclopropanecarboxylic acid (1.5 equiv, 31 mg) in DCM (3 mL) followed by a drop of DMF. After stirring at room temperature for 40 min, pyridine (2 mL) and the azetidine derivative (1 equiv, 100 mg) and the mixture stirred at room temperature for 16 h. The reaction mixture was diluted with DCM, washed with aq. sat. NaHCO₃ solution, dried and concentrated. Purification by silica chromatography (7M NH₃ solution in MeOH/EtOAc; 10:90) afforded the desired compound.

### Compound 349: {2-Ethyl-4-[1-(2-methoxy-ethanesulfonyl)-azetidin-3-yl]-phenyl}-methyl-(1-methyl-1H-imidazo[4,5-c]pyridin-6-yl)-amine

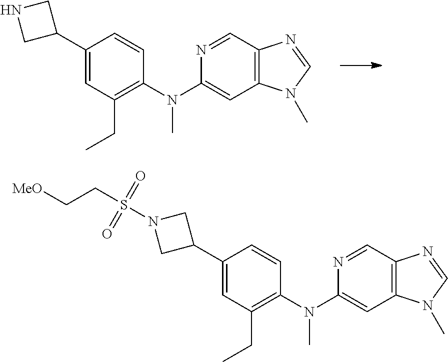

2-Methoxy-ethanesulfonyl chloride (1.1 equiv, 33 mg) was added to a solution of the azetidine derivative (1 equiv, 100 mg) and pyridine (2 mL) in DCM (3 mL) at 0° C. and the mixture was stirred at room temperature for 16 h. The reaction mixture was diluted with DCM, washed with aq. sat. NaHCO₃ solution, dried and concentrated. Purification by silica chromatography (EtOAc) afforded the desired compound.

### Compound 350:1-(3-{3-Ethyl-4-[methyl-(1-methyl-1H-imidazo[4,5-c]pyridin-6-yl)-amino]-phenyl}-azetidin-1-yl)-2-methoxy-ethanone

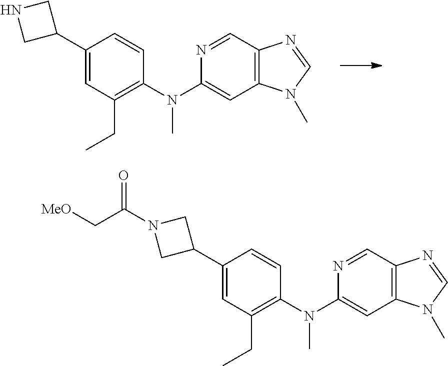

Methoxy-acetyl chloride (1.3 equiv, 27 mg) was added to a solution of the azetidine derivative (1 equiv, 100 mg) and pyridine (2 mL) in DCM (3 mL) and the mixture was stirred at room temperature for 1 h. The reaction mixture was diluted with DCM, washed with aq. sat. NaHCO₃ solution, dried and concentrated. Purification by silica chromatography (2N NH₃ solution in MeOH/EtOAc; 10:90) afforded the desired compound.

### Compound 351: (3-{3-Ethyl-4-[methyl-(1-methyl-1H-imidazo[4,5-c]pyridin-6-yl)-amino]-phenyl}-azetidin-1-yl)-acetonitrile

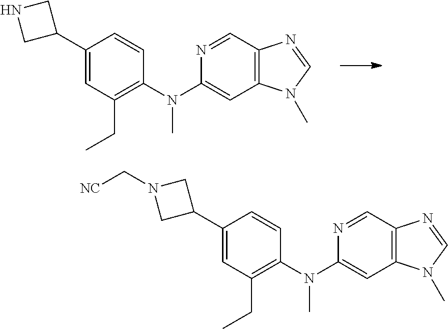

Sodium hydride (60%, 1.1 equiv, 8 mg) was added to the azetidine derivative (1 equiv, 100 mg) in THF (2 mL) After 5 min, bromoacetonitrile (1.3 equiv, 30 mg) was added and the solution was stirred at 60° C. for 16 h. The reaction mixture was quenched with water and concentrated to dryness. Purification by silica chromatography (2N NH₃ solution in MeOH/EtOAc; 0:100 to 10:90) afforded the desired compound.

### Compound 352: {2-Ethyl-4-[1-(2,2,2-trifluoro-ethanesulfonyl)-azetidin-3-yl]-phenyl}-methyl-(1-methyl-1H-imidazo[4,5-c]pyridin-6-yl)-amine

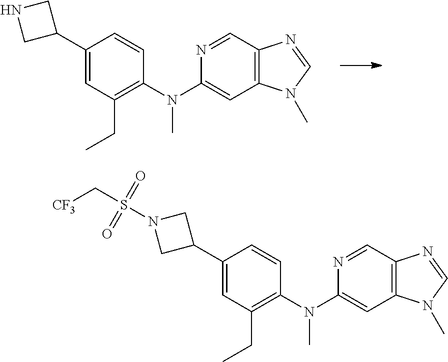

Trifluoro-ethanesulfonyl chloride (1.1 equiv, 401 mg) was added dropwised to a solution of the azetidine derivative (1 equiv, 644 mg) and pyridine (1 mL) in DCM (2 mL) at 0° C. After 1 h, the reaction mixture was diluted with DCM, washed with aq. sat. NaHCO₃ solution, dried and concentrated. Purification by silica chromatography (EtOAc/petrol; 90:10 then MeOH/EtOAc; 5:95) afforded the desired compound.

### Compound 353: (3-{3-Ethyl-4-[methyl-(1-methyl-1H-imidazo[4,5-c]pyridin-6-yl)-amino]-phenyl}-azetidine-1-sulfonyl)-acetonitrile

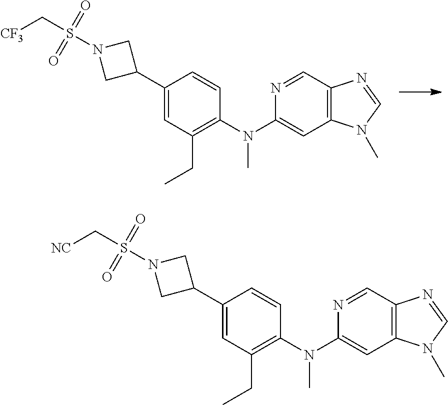

A solution of trifluoroethanesulfonamide (1 equiv, 740 mg) in 7N NH₃ in MeOH (20 mL) was stirred at room temperature for 1.5 h. The mixture was concentrated, rediluted in DCM and filtered. The filtrate was concentrated and the residue purified by silica chromatography (EtOAc; 100) to give the desired compound.

### Compound 354: {4-[1-(Difluoro-methanesulfonyl)-azetidin-3-yl]-2-ethyl-phenyl}-methyl-(1-methyl-1H-imidazo[4,5-c]pyridin-6-yl)-amine

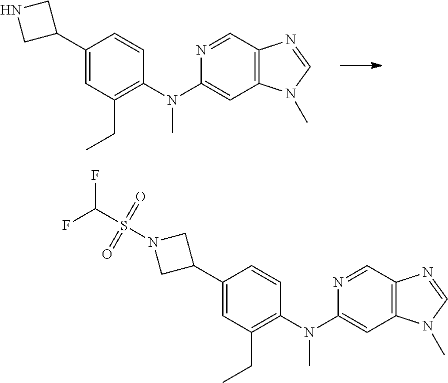

Difluoro-methanesulfonyl chloride (1.1 equiv, 102 mg) was added dropwised to a solution of the azetidine derivative (1 equiv, 200 mg) and pyridine (1 mL) in DCM (1 mL) at 0° C. After 5 min, the reaction mixture was diluted with DCM, washed with aq. sat. NaHCO₃, dried and concentrated. Purification by silica chromatography (EtOAc/petrol; 65:35 to 90:10) afforded the desired compound.

### Compound 355: N-Cyanomethyl-N-{3-fluoro-4-[methyl-(1-methyl-1H-imidazo[4,5-c]pyridin-6-A-amino]-benzyl}-methanesulfonamide

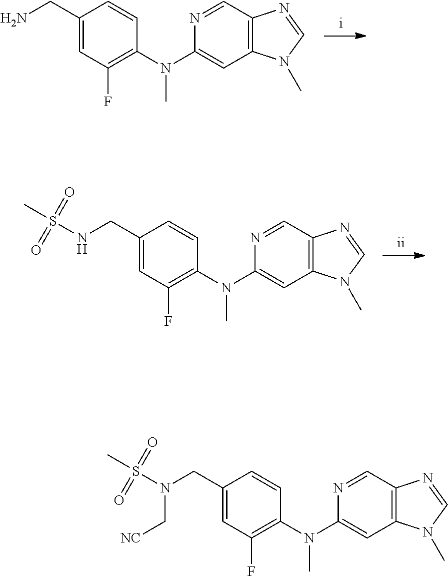 [SMILES: CN(c1cc2c(cn1)ncn2C)c1ccc(CN)cc1F ; CN(c1cc2c(cn1)ncn2C)c1ccc(CNS(C)(=O)=O)cc1F ; CN(c1cc2c(cn1)ncn2C)c1ccc(CN(CC#N)S(C)(=O)=O)cc1F]

### Step (i): (N-{3-Fluoro-4-[methyl-(1-methyl-1H-imidazo[4,5-c]pyridin-6-yl)-amino]-benzyl}-methane-sulfonamide

Triethylamine (1 equiv, 0.49 mL) was added. dropwised to a solution of the benzylamine (1 equiv, 1.01 g) and methanesulfonyl chloride (1 equiv, 0.27 mL) in THF (40 mL) at 0° C. The mixture was then stirred at room temperature until completion. The mixture was diluted with water and the compound was extracted with EtOAc, dried and concentrated. Purification by silica chromatography (MeOH:DCM; 0.5:99.5 to 10:90) afforded the desired compound.

### Step (ii): N-Cyanomethyl-N-{3-fluoro-4-[methyl-(1-methyl-1H-imidazo[4,5-c]pyridin-6-yl)-amino]-benzyl}-methanesulfonamide

Sodium hydride (1.5 equiv, 20 mg) was added to a solution of (N-{3-fluoro-4-[methyl-(1-methyl-1H-imidazo[4,5-c]pyridin-6-yl)-amino]-benzyl}-methanesulfonamide (1 equiv, 119 mg) in THF (1 mL) at 0° C. After 30 min, bromoacetonitrile (1.5 equiv, 34 μL) was added and the reaction was stirred at room temperature for 16 h. The mixture was diluted with EtOAc, washed with water, dried and concentrated. Purification by silica chromatography (MeOH/DCM; 0:100 to 10:90) afforded the desired compound.

### Compound 356: N-{3-Ethyl-5-fluoro-4-[methyl-(1-methyl-M-imidazo[4,5-c]pyridin-6-yl)-amino]-benzyl}-N-methyl-methanesulfonamide

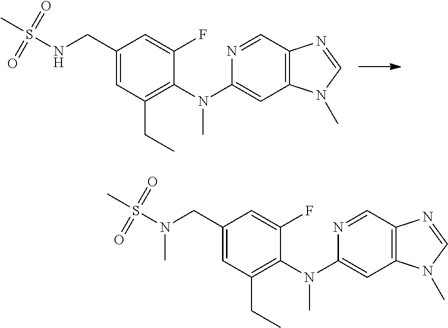

Sodium hydride (1.5 equiv, 10 mg) was added to a solution of N-{3-ethyl-5-fluoro-4-[methyl-(1-methyl-1H-imidazo[4,5-c]pyridin-6-yl)-amino]-benzyl}-methane sulfonamide (1 equiv, 60 mg) in THF (1 mL) at 0° C. After 30 min, methyl iodide (1.5 equiv, 15 μL) was added and the reaction was stirred at room temperature for 4 h. The mixture was diluted with EtOAc, washed with water, dried and concentrated. Purification preparative HPLC afforded the desired compound.

### Compound 357: N-Cyanomethyl-N-{3-ethyl-5-fluoro-4-[methyl-(1-methyl-1H-imidazo[4,5-c]pyridin-6-yl)-amino]-benzyl}-methanesulfonamide

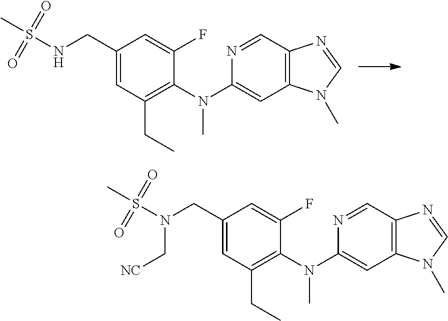

Sodium hydride (1.5 equiv, 10 mg) was added to a solution of N-{3-ethyl-5-fluoro-4-[methyl-(1-methyl-1H-imidazo[4,5-c]pyridin-6-yl)-amino]-benzyl}-methane sulfonamide (1 equiv, 60 mg) in THF (1 mL) at 0° C. After 30 min, iodoacetonitrile (1.5 equiv, 17 μL) was added and the reaction was stirred at room temperature for 4 h. The mixture was diluted with EtOAc, washed with water, dried and concentrated. Purification preparative HPLC afforded the desired compound.

### Compound 358: N-{3-Fluoro-4-[methyl-(1-methyl-1H-imidazo[4,5-c]pyridin-6-yl)-amino]-benzyl}-N-methyl-methanesulfonamide

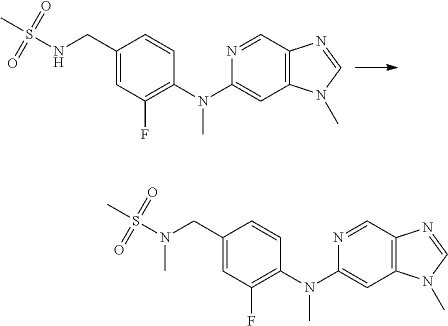 [SMILES: CN(c1cc2c(cn1)ncn2C)c1ccc(CNS(C)(=O)=O)cc1F ; CN(c1cc2c(cn1)ncn2C)c1ccc(CN(C)S(C)(=O)=O)cc1F]

Sodium hydride (1.5 equiv, 20 mg) was added to a solution of N-{3-fluoro-4-[methyl-(1-methyl-1H-imidazo[4,5-c]pyridin-6-yl)-amino]-benzyl}-methanesulfonamide (1 equiv, 119 mg) in THF (1 mL) at 0° C. After 30 min, methyl iodide (1.5 equiv, 31 μL) was added and the reaction was stirred at room temperature for 16 h. The mixture was diluted with EtOAc, washed with water, dried and concentrated. Purification by silica chromatography (MeOH/DCM; 0:100 to 10:90) afforded the desired compound.

### Compound 359: N-{3-Fluoro-4-[methyl-(1-methyl-1H-imidazo[4,5-c]pyridin-6-yl)-amino]-benzyl}-N-methoxymethyl-methanesulfonamide

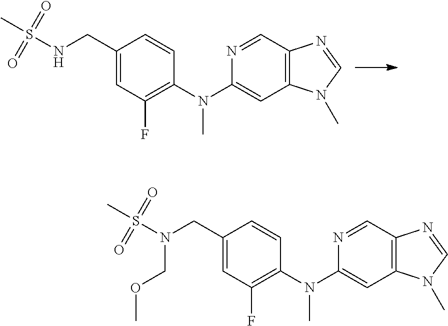 [SMILES: CN(c1cc2c(cn1)ncn2C)c1ccc(CNS(C)(=O)=O)cc1F ; COCN(Cc1ccc(N(C)c2cc3c(cn2)ncn3C)c(F)c1)S(C)(=O)=O]

Sodium hydride (1.5 equiv, 20 mg) was added to a solution of N-{3-fluoro-4-[methyl-(1-methyl-1H-imidazo[4,5-c]pyridin-6-yl)-amino]-benzyl}-methanesulfonamide (1 equiv, 119 mg) in THF (1 mL) at 0° C. After 30 min, bromo-methoxy-methane (1.5 equiv, 40 μL) was added and the reaction was stirred at room temperature for 16 h. The mixture was diluted with EtOAc., washed with water, dried and concentrated. Purification by silica chromatography (MeOH/DCM; 0:100 to 10:90) afforded the desired compound.

### Compound 360: N-{3-Fluoro-4-[methyl-(1-methyl-1H-imidazo[4,5-c]pyridin-6-yl)-amino]-benzyl}-N-methoxymethyl-methanesulfonamide

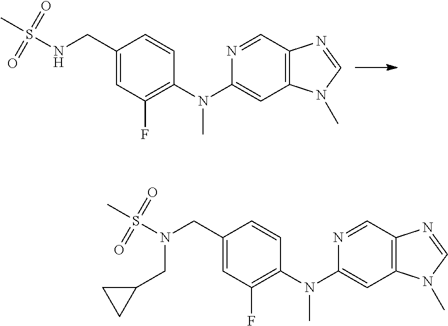 [SMILES: CN(c1cc2c(cn1)ncn2C)c1ccc(CNS(C)(=O)=O)cc1F ; CN(c1cc2c(cn1)ncn2C)c1ccc(CN(CC2CC2)S(C)(=O)=O)cc1F]

Sodium hydride (1.5 equiv, 20 mg) was added to a solution of N-{3-fluoro-4-[methyl-(1-methyl-1H-imidazo[4,5-c]pyridin-6-yl)-amino]-benzyl}-methanesulfonamide (1 equiv, 119 mg) in THF (1 mL) at 0° C. After 30 min, bromomethyl-cyclopropane (1.5 equiv, 43 μL) was added and the reaction was stirred at room temperature until completion. The mixture was diluted with EtOAc, washed with water, dried and concentrated. Purification by silica chromatography (MeOH/DCM; 0:100 to 10:90) afforded the desired compound.

### Compound 362 and 363: 3-{3-Difluoromethoxy-4-[methyl-(1-methyl-1H-imidazo[4,5-]pyridin-6-yl)-amino]-phenyl}-azetidine-1-carboxylic acid tert-butyl ester and [2-Difluoromethoxy-4-(1-methanesulfonyl-azetidin-3-yl)-phenyl]-ethyl-(1-methyl-1H-imidazo[4,5-c]pyridin-6-yl)-amine

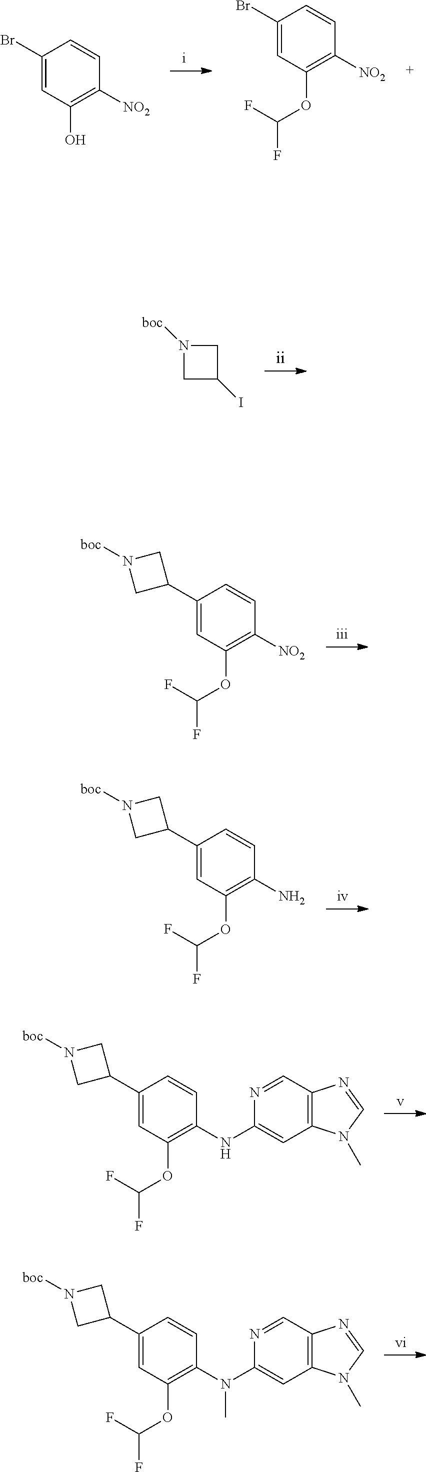

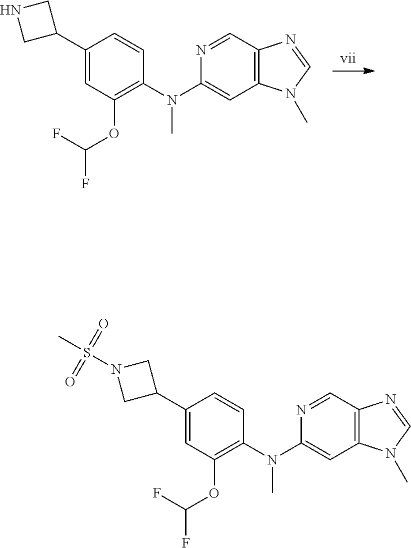 [SMILES: CN(c1cc2c(cn1)ncn2C)c1ccc(C2CNC2)cc1OC(F)F ; CN(c1cc2c(cn1)ncn2C)c1ccc(C2CN(S(C)(=O)=O)C2)cc1OC(F)F]

### Step (i): 4-Bromo-2-difluoromethoxy-1-nitro-benzene

The phosphonate (2 equiv, 8.2 mL) was added to a solution of 5-bromo-2-nitro-phenol (1 equiv, 5.0 g) and KOH (20 equiv, 25 g) in MeCN (115 mL) and water (115 mL) at −30° C. The reaction was then allowed to warm to room temperature. After 1 h, the mixture was diluted with Et₂O, the layer separated and the aqueous extracted with more Et₂O. The combined organics was dried and concentrated to give the desired compound.

### Step (ii): 3-(3-Difluoromethoxy-4-nitro-phenyl)-azetidine-1-carboxylic acid tert-butyl ester

N-Boc-azetidine-iodide (2.5 equiv, 2.7 g) was dissolved in DMA (6 mL) and heated to 65° C. Under atmosphere of argon, Rieke zinc (2.5 equiv, 12.5 mL) was added dropwise over 20 min and stirred for 10 more min. In another flask 4-bromo-2-fluoronitrobenzene (1 equiv 1.0 g), copper(I) iodide (0.06 equiv, 45 mg) and PdCl₂dppf (0.03 equiv, 100 mg) were dissolved in DMA (2 mL) and heated to 85° C. under argon. The reaction mixture from the first flask was added dropwise to second flask over 20 min with a cannula and the resulting mixture was stirred at 85° C. for 18 h. The reaction was then quenched with aq. sat. NH₄Cl solution and extracted with EtOAc (3×). The organics were combined, dried (Na₂SO₄) and concentrated under reduced pressure. The residue was purified by silica chromatography (EtOAc/petrol ether; 0:100 to 20:80) to give the desired compound.

### Step (iii): 3-(4-Amino-3-difluoromethoxy-phenyl)-azetidine-1-carboxylic acid tert-butyl ester

HCO₂H (5 mL) was added to a mixture of the nitroaryl (1 equiv, 2.2 g), Zn powder (10 equiv, 2.45 g) and NH₄Cl (catalytic amount) in MeOH (20 mL) and the reaction was stirred at 80° C. After 30 min, the reaction mixture was filtered and the filtrate concentrated. The residue was diluted with DCM, washed with aq. sat. NaHCO₃ solution, dried and concentrated. Purification by silica chromatography (EtOAc/petrol ether; 0:100 to 30:70) afforded the desired compound.

### Step (iv): 3-[3-Difluoromethoxy-4-(1-methyl-1H-imidazo[4,5-c]pyridin-6-ylamino)-phenyl]-azetidine-1-carboxylic acid tert-butyl ester

A mixture of the aniline derivative (1 equiv, 670 mg), 6-chloro-1-methyl-1H-imidazo[4,5-c]pyridine (1.15 equiv, 410 mg), BINAP (0.3 equiv, 400 mg), Cs₂CO₃ (3.0 equiv, 2.08 g) and tris(dibenzylideneacetone)dipalladium(0) (0.2 equiv, 390 mg) in dry 1,4-dioxane (5 mL), degassed under (N₂), was heated at 100° C. for 5 h. The solution was diluted with DCM, filtered through Celite and the filtrate was concentrated.

### Step (v): 3-{3-Difluoromethoxy-4-[methyl-(1-methyl-1H-imidazo[4,5-c]pyridin-6-yl)-amino]-phenyl}-azetidine-1-carboxylic acid tert-butyl ester

Sodium hydride (60%, 3 equiv, 110 mg) was added to a solution of the amine (1 equiv, 1.8 g) in THF (10 mL) followed by the addition of MeI (3 equiv, 0.28 mL) and the reaction mixture was stirred at room temperature. After 1 h, water was added and the solution extracted with EtOAc, dried and concentrated. Purification by silica chromatography (EtOAc/petrol ether; 50:50 to 100:0 then MeOH/EtOAc; 0:100 to 5:95) delivered the expected compound.

### Step (vi): (4-Azetidin-3-yl-2-difluoromethoxy-phenyl)-methyl-(1-methyl-1H-imidazo[4,5-c]pyridin-6-yl)-amine

Trifluoroacetic acid (5 mL) was added to a solution of the Boc-protected azetidine derivative (1 equiv, 350 mg) in DCM (5 mL) and the mixture was stirred at room temperature. After 1.5 h, the mixture was diluted with DCM, washed with water, dried and concentrated.

### Step (vi): [2-Difluoromethoxy-4-(1-methanesulfonyl-azetidin-3-yl)-phenyl]-ethyl-(1-methyl-1H-imidazo[4,5-c]pyridin-6-yl)-amine

Methanesulfonyl chloride (1.1 equiv, 25 μg) was slowly added to a solution of the azetidine derivative (1 equiv, 100 mg) and DIPEA (3 equiv, 140 μL) in DCM (1 mL) at 0° C. After 30 min, the reaction mixture was diluted with DCM, washed with water, dried and concentrated. Purification by silica chromatography (MeOH/DCM; 0:100 to 5:95) afforded the desired compound.

### Compound 364: {2-Difluoromethoxy-4-[1-(2,2,2-trifluoro-ethyl)-azetidin-3-yl]-phenyl}-methyl-(1-methyl-1H-imidazo[4,5-c]pyridin-6-yl)-amine

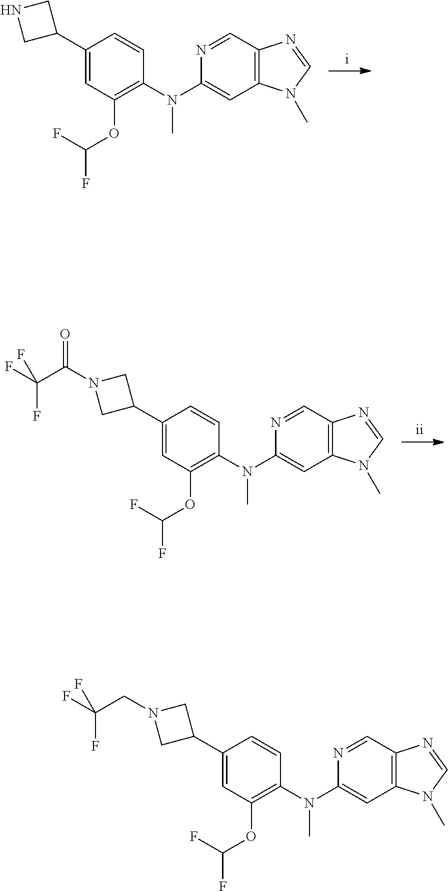 [SMILES: CN(c1cc2c(cn1)ncn2C)c1ccc(C2CNC2)cc1OC(F)F ; CN(c1cc2c(cn1)ncn2C)c1ccc(C2CN(C(=O)C(F)(F)F)C2)cc1OC(F)F ; CN(c1cc2c(cn1)ncn2C)c1ccc(C2CN(CC(F)(F)F)C2)cc1OC(F)F]

### Step (i): 1-(3-{3-Difluoromethoxy-4-[methyl-(1-methyl-1H-imidazo[4,5-c]pyridin-6-yl)-amino]-phenyl}-azetidin-1-yl)-2,2,2-trifluoro-ethanone

Trifluoroacetic acid (1.05 equiv, 148 μL) was slowly added to a solution of the azetidine derivative (1 equiv, 50 mg) and DIPEA (3 equiv, 73 μL) in DCM at 0° C. and the reaction was stirred at room temperature for 1.5 h. The reaction mixture was then diluted with DCM, washed with water, dried and concentrated.

### Step (ii): {2-Difluoromethoxy-4-[1-(2,2,2-trifluoro-ethyl)-azetidin-3-yl]-phenyl}-methyl-(1-methyl-1H-imidazo[4,5-c]pyridin-6-yl)-amine

Borane (1.0 M in THF, 4 equiv, 0.88 mL) was added to a solution of the amide (1 equiv, 100 mg) in THF (5 mL) and the mixture was heated at 50° C. After 2 h, the mixture was diluted with DCM, washed with water, dried and concentrated. Purification by preparative HPLC afforded the desired compound.

### Compound 365: {4-[1-(2,2-Difluoro-ethyl)-azetidin-3-yl]-2-difluoromethoxy-phenyl}-methyl-(1-methyl-1H-imidazo[4,5-c]pyridin-6-yl)-amine

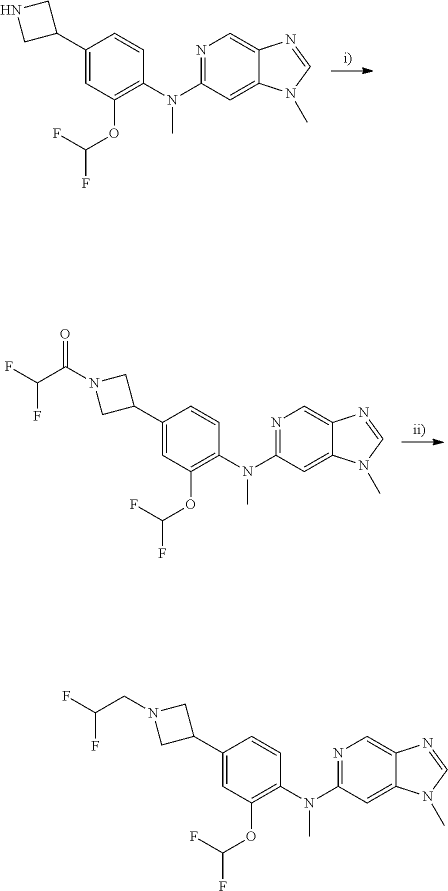 [SMILES: CN(c1cc2c(cn1)ncn2C)c1ccc(C2CNC2)cc1OC(F)F ; CN(c1cc2c(cn1)ncn2C)c1ccc(C2CN(C(=O)C(F)F)C2)cc1OC(F)F ; CN(c1cc2c(cn1)ncn2C)c1ccc(C2CN(CC(F)F)C2)cc1OC(F)F]

Synthesis followed the same conditions used for compound 364.

### Compound 366: Azetidin-1-yl-(3-{3-difluoromethoxy-4-[methyl-(1-methyl-1H-imidazo[4,5-c]pyridin-6-yl)-amino]-phenyl}-azetidin-1-yl)-methanone

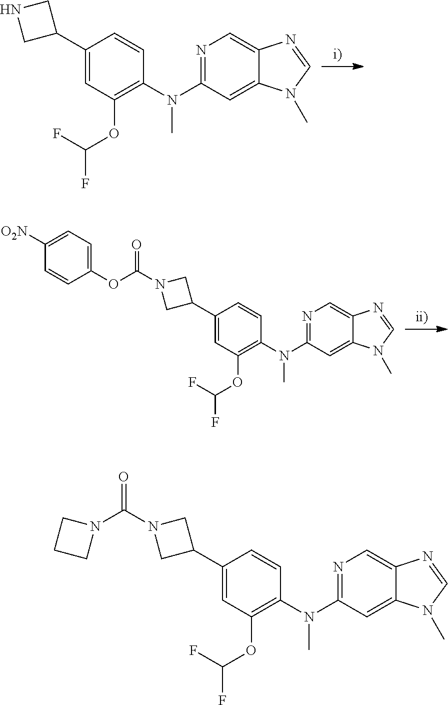 [SMILES: CN(c1cc2c(cn1)ncn2C)c1ccc(C2CNC2)cc1OC(F)F ; CN(c1cc2c(cn1)ncn2C)c1ccc(C2CN(C(=O)Oc3ccc([N+](=O)[O-])cc3)C2)cc1OC(F)F ; CN(c1cc2c(cn1)ncn2C)c1ccc(C2CN(C(=O)N3CCC3)C2)cc1OC(F)F]

### Step (i): 3-{3-Difluoromethoxy-4-[methyl-(1-methyl-1H-imidazo[4,5-c]pyridin-6-yl)-amino]-phenyl}-azetidine-1-carboxylic acid 4-nitro-phenyl ester

4-Nitrophenylchloroformate (1.5 equiv, 340 mg) was added to a solution of the azetidine derivative (1 equiv, 500 mg), and DIPEA (2 equiv, 0.49 mL) in DCM (5 mL) and the reaction mixture was stirred room temperature for 30 min. The solution was diluted with DCM, washed with aq. sat. NaHCO₃ solution, dried and concentrated. Purification by silica chromatography (MeOH/DCM, 0:100 to 10:90) afforded the desired product.

### Step (ii): Azetidin-1-yl-(3-{3-difluoromethoxy-4-[methyl-(1-methyl-M-imidazo[4,5-c]pyridin-6-yl)-amino]-phenyl}-azetidin-1-yl)-methanone

A mixture of the nitrophenylester (1 equiv, 100 mg), azetidine derivative (1.3 equiv, 15 mg) and DIPEA (2 equiv, 70 μL) in DCM (1 mL) was stirred at room temperature for 5 h. The mixture was diluted with DCM washed with sat. NaHCO₃ solution, dried and concentrated. Purification by preparative HPLC gave the desired compound.

### Compound 367: (3-{3-Difluoromethoxy-4-[methyl-(1-methyl-1H-imidazo[4,5-c]pyridin-6-yl)-amino]-phenyl}-azetidin-1-yl)-piperidin-1-yl-methanone

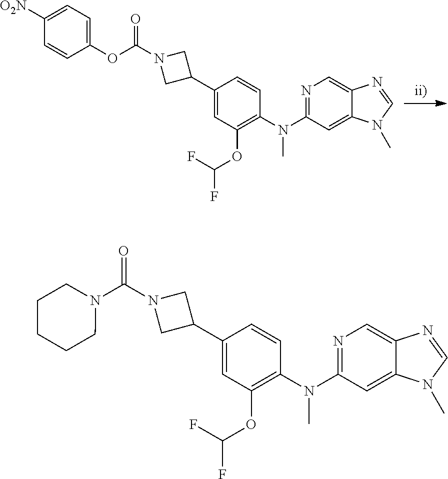 [SMILES: CN(c1cc2c(cn1)ncn2C)c1ccc(C2CN(C(=O)Oc3ccc([N+](=O)[O-])cc3)C2)cc1OC(F)F ; CN(c1cc2c(cn1)ncn2C)c1ccc(C2CN(C(=O)N3CCCCC3)C2)cc1OC(F)F]

A mixture the nitrophenylester (1 equiv, 100 mg), piperidine (1.3 equiv, 25 μL) and DIPEA (2 equiv, 70 μL) in DCM (1 mL) was stirred at room temperature for 5 h. The misture was diluted with DCM, washed with aq. sat. NaHCO₃ solution, dried and concentrated. Purification by preparative HPLC afforded the desired compound.

### Compound 368: (3-{3-Difluoromethoxy-4-[methyl-(1-methyl-1H-imidazo[4,5-c]pyridin-6-yl)-amino]-phenyl}-azetidin-1-yl)-piperidin-1-yl-methanone

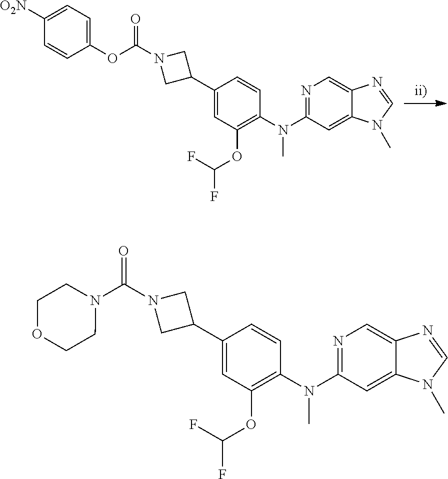 [SMILES: CN(c1cc2c(cn1)ncn2C)c1ccc(C2CN(C(=O)Oc3ccc([N+](=O)[O-])cc3)C2)cc1OC(F)F ; CN(c1cc2c(cn1)ncn2C)c1ccc(C2CN(C(=O)N3CCOCC3)C2)cc1OC(F)F]

A mixture the nitrophenylester (1 equiv, 100 mg), morpholine (1.3 equiv, 22 μL) and DIPEA (2 equiv, 70 μL) in DCM (1 mL) was stirred at room temperature for 5 h. The mixture was diluted with DCM, washed with aq. sat. NaHCO₃ solution, dried and concentrated. Purification by preparative HPLC afforded the desired compound.

### Compound 369: 3-{3-Difluoromethoxy-4-[methyl-(1-methyl-1H-imidazo[4,5-c]pyridin-yl)-amino]-phenyl}-azetidine-1-sulfonic acid dimethylamide

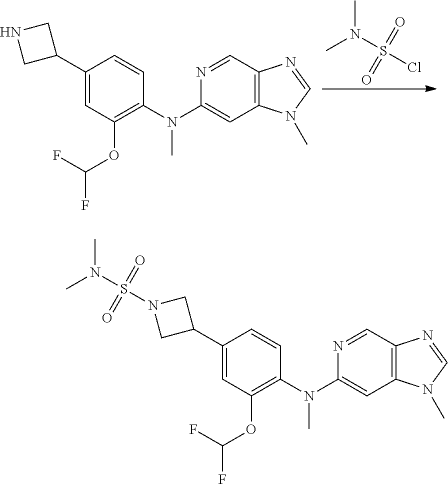

The corresponding sulfamoyl chloride (1.1 equiv, 16 μL) in DCM (0.5 mL) was slowly added to a solution of the azetidine derivative (1 equiv, 50 mg) and DIPEA (3 equiv, 75 μL) in DCM (0.5 mL) at 0° C. After 1 h, the reaction mixture was diluted with DCM, washed with water, dried and concentrated. Purification by preparative HPLC afforded the desired compound.

### Compound 370: {4-[1-(Difluoro-methanesulfonyl)-azetidin-3-yl]-2-difluoromethoxy-henyl}-methyl-(1-methyl-1H-imidazo[4,5-c]pyridin-6-yl)-amine

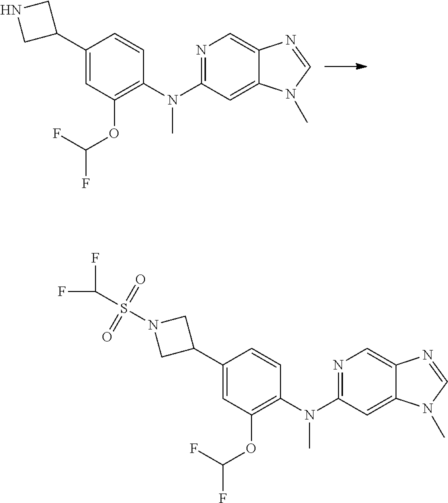 [SMILES: CN(c1cc2c(cn1)ncn2C)c1ccc(C2CNC2)cc1OC(F)F ; CN(c1cc2c(cn1)ncn2C)c1ccc(C2CN(S(=O)(=O)C(F)F)C2)cc1OC(F)F]

The difluoro-methanesulfonyl chloride (1.1 equiv, 30 μL) in DCM (1 mL) was slowly added to a solution of the azetidine (1 equiv, 100 mg) and DIPEA (2 equiv, 100 μL) in DCM (1 mL) at 0° C. After 1 h, the reaction mixture was diluted with DCM, washed with water, dried and concentrated. Purification by preparative HPLC afforded the desired compound.

### Compound 371: {2-Difluoromethoxy-4-[1-(morpholine-4-sulfonyl)-azetidin-3-yl]-phenyl}-methyl-(1-methyl-1H-imidazo[4,5-c]pyridin-6-yl)-amine

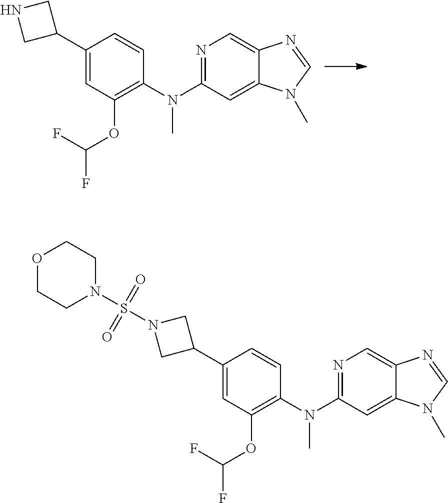 [SMILES: CN(c1cc2c(cn1)ncn2C)c1ccc(C2CNC2)cc1OC(F)F ; CN(c1cc2c(cn1)ncn2C)c1ccc(C2CN(S(=O)(=O)N3CCOCC3)C2)cc1OC(F)F]

Morpholine-4-sulfonyl chloride (1.1 equiv, 204 μL) in DCM (1 mL) was slowly added to a solution of the azetidine derivative (1 equiv, 100 mg) and DIPEA (2 equiv, 100 μL) in DCM (1 mL) at 0° C. After 1 h, the reaction mixture was diluted with DCM, washed with water, dried and concentrated. Purification by preparative HPLC afforded the desired compound.

### Compound 372: {2-Difluoromethoxy-4-[1-(piperidine-1-sulfonyl)-azetidin-3-yl]-phenyl}-methyl-(1-methyl-1H-imidazo[4,5-c]pyridin-6-yl)-amine

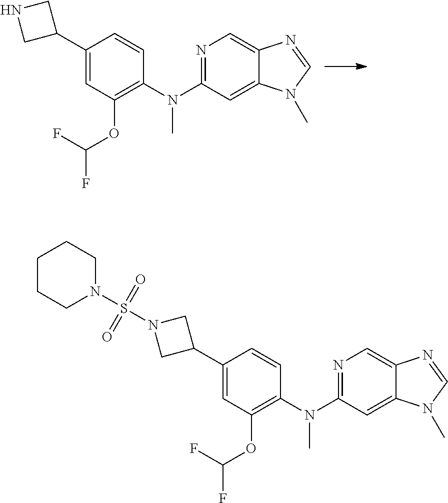 [SMILES: CN(c1cc2c(cn1)ncn2C)c1ccc(C2CNC2)cc1OC(F)F ; CN(c1cc2c(cn1)ncn2C)c1ccc(C2CN(S(=O)(=O)N3CCCCC3)C2)cc1OC(F)F]

Piperidine-1-sulfonyl chloride (1.1 equiv, 202 μL) in DCM (1 mL) was slowly added to a solution of the azetidine derivative (1 equiv, 100 mg) and DIPEA (3 equiv, 100 μL) in DCM (1 mL) at 0° C. After 1 h, the reaction mixture was diluted with DCM, washed with water, dried and concentrated. Purification by preparative HPLC afforded the desired compound.

### Compound 373: {2-Difluoromethoxy-4-[1-(propane-2-sulfonyl)-azetidin-3-yl]-phenyl}-methyl-(1-methyl-1H-imidazo[4,5-c]pyridin-6-yl)-amine

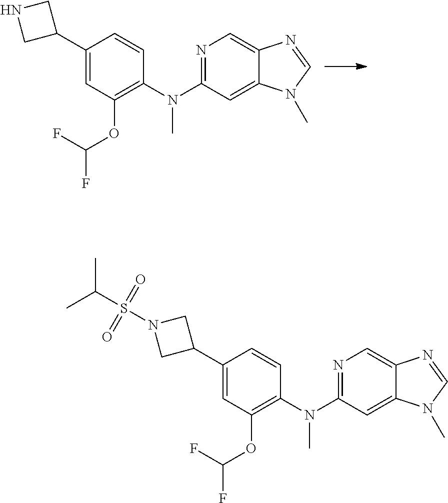 [SMILES: CN(c1cc2c(cn1)ncn2C)c1ccc(C2CNC2)cc1OC(F)F ; CC(C)S(=O)(=O)N1CC(c2ccc(N(C)c3cc4c(cn3)ncn4C)c(OC(F)F)c2)C1]

Propane-2-sulfonyl chloride (1.5 equiv, 50 μL) in DCM (1 mL) was slowly added to a solution of the azetidine derivative (1 equiv, 100 mg) and DIPEA (3 equiv, 150 μL) in DCM (1 mL) at 0° C. After 1 h, the reaction mixture was diluted with DCM, washed with water, dried and concentrated. Purification by preparative HPLC afforded the desired compound.

### Compound 374: 2-(3-{3-Difluoromethoxy-4-[methyl-(1-methyl-1H-imidazo[4,5-c]pyridin-6-yl)-amino]-phenyl}-azetidin-1-yl)-acetamide

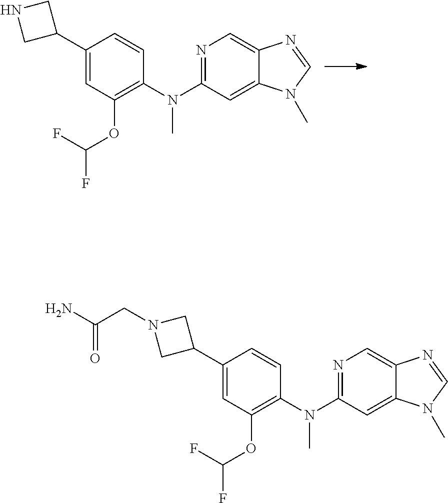 [SMILES: CN(c1cc2c(cn1)ncn2C)c1ccc(C2CNC2)cc1OC(F)F ; CN(c1cc2c(cn1)ncn2C)c1ccc(C2CN(CC(N)=O)C2)cc1OC(F)F]

To a solution of the azetidine derivative (1 equiv, 100 mg) and NaH (2 equiv, 25 mg) in dry THF (1 mL) was added the chloroacetamide (1.5 equiv, 40 mg) at 0° C. The mixture was then allowed to warm to room temperature. After 30 min, water was added and the mixture extracted with EtOAc. The organic layer was washed with aq. sat. NaHCO₃ solution, dried and concentrated. Purification by preparative HPLC afforded the desired compound.

### Compound 375

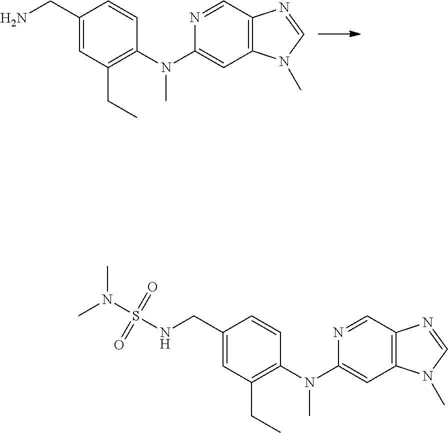 [SMILES: CCc1cc(CN)ccc1N(C)c1cc2c(cn1)ncn2C ; CCc1cc(CNS(=O)(=O)N(C)C)ccc1N(C)c1cc2c(cn1)ncn2C]

Triethylamine (1.5 equiv, 71 μL) was added slowly to a solution of the benzylamine derivative (1 equiv, 100 mg) and dimethylsulfamoyl chloride (1.5 equiv, 54 μL) in THF (5 mL) at 0° C. and the reaction mixture was stirred for 18 h at room temperature. The mixture was concentrated and purification by preparative HPLC afforded the desired compound.

### Compound 376

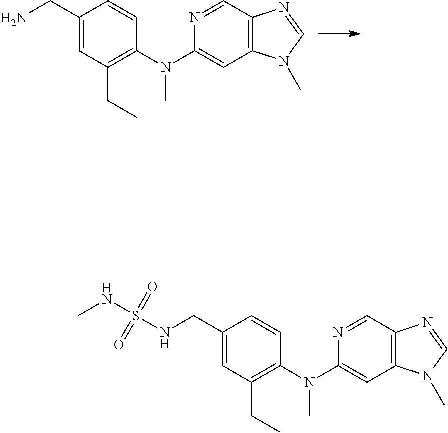 [SMILES: CCc1cc(CN)ccc1N(C)c1cc2c(cn1)ncn2C ; CCc1cc(CNS(=O)(=O)NC)ccc1N(C)c1cc2c(cn1)ncn2C]

triethylamine (1 equiv, 47 μL) was added slowly to a solution of the benzylamine derivative (1 equiv, 100 mg) and dimethylsulfamoyl chloride (1 equiv, 29 μL) in THF (5 mL) at 0° C. and the reaction mixture was stirred for 18 h at room temperature. The mixture was concentrated and purification by preparative HPLC afforded the desired compound.

### Compound 377: 4,4-Difluoro-cyclohexanecarboxylic acid 3-ethyl-4-[methyl-(1-methyl-1H-imidazo[4,5-c]pyridin-6-yl)-amino]-benzylamide

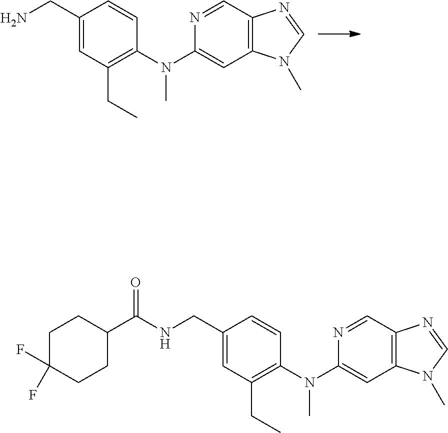 [SMILES: CCc1cc(CN)ccc1N(C)c1cc2c(cn1)ncn2C ; CCc1cc(CNC(=O)C2CCC(F)(F)CC2)ccc1N(C)c1cc2c(cn1)ncn2C]

N-Methylmorpholine (4 equiv, 75 μL), TBTU (2 equiv, 109 mg) and 4,4-difluoro-cyclohexanecarboxylic acid (1 equiv, 28 mg) were added to the benzylamine derivative (1 equiv, 50 mg) in THF (4 mL) and the mixture was heated at 60° C. After 16 h, the solvent was evaporated, water was added and the product was extracted with DCM. The organic layer was dried and concentrated. Solvent was evaporated under reduced pressure. Purification by preparative HPLC afforded the desired compound.

### Compound 378: [4-(3,3-Difluoro-azetidin-1-ylmethyl)-2-ethyl-phenyl]-methyl-(1-methyl-1H-imidazo[4,5-c]pyridin-6-yl)-amine

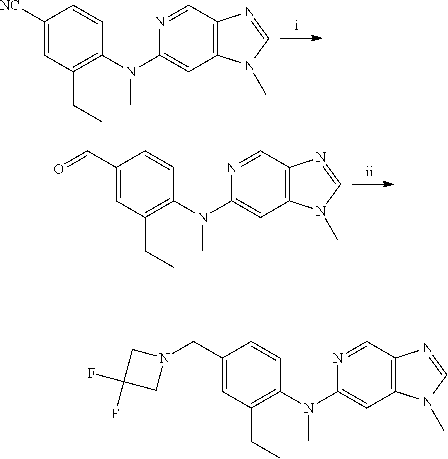

### Step (i): 3-Ethyl-4-[methyl-(1-methyl-1H-imidazo[4,5-c]pyridin-6-yl)-amino]-benzaldehyde

Dibal-H (1M in cyclohexane, 3 equiv, 1 mL) was added dropwised to a solution of the benzylamine (1 equiv, 100 mg) in toluene (10 mL) at −78° C. After 1.5 h, the mixture was warmed up at 0° C., Rochelle's salt (1 mL) was added and the reaction mixture was stirred at 0° C. for 30 min. The solution was diluted with EtOAc, the layer separated and the organics dried and concentrated. The residue was rediluted in THF (3 mL), aq. 2M HCl solution (2 mL) was added and the mixture was stirred at room temperature for 2 h. The mixture was brought to pH: 5-6 and product was extracted with ethyl acetate, dried on Na₂SO₄, filtered and concentrated. Purification by silica flash chromatography (MeOH/EtOAc) gave the product.

### Step (ii): [4-(3,3-Difluoro-azetidin-1-ylmethyl)-2-ethyl-phenyl]-methyl-(1-methyl-1H-imidazo[4,5-e]pyridin-6-yl)-amine

A solution of the aldehyde (1 equiv, 80 mg), 3,3-difluoro-azetidine hydrochloride (1.3 equiv, 46 mg) and AcOH (136 μL) in MeOH (4 mL) and DCM (2.7 mL) was stirred at room temperature for 3 h. Na(CN)BH₃ (0.5 equiv, 9 mg) was added and the mixture was stirred at room temperature. After 2 h, the reaction was concentrated and purification by preparative HPLC afforded the desired compound.

### Compound 379: {4-[(2,2-Difluoro-ethylamino)-methyl]-2-ethyl-phenyl}-methyl-(1-ethyl-1H-imidazo[4,5-c]pyridin-6-yl)-amine

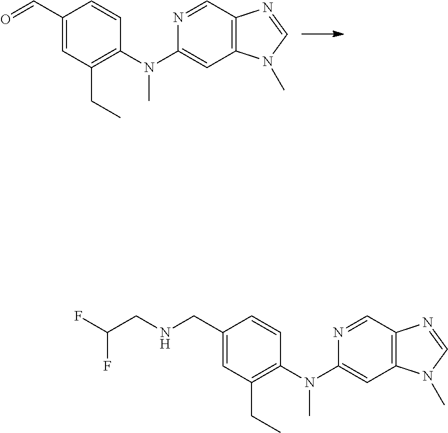 [SMILES: CCc1cc(C=O)ccc1N(C)c1cc2c(cn1)ncn2C ; CCc1cc(CNCC(F)F)ccc1N(C)c1cc2c(cn1)ncn2C]

A solution of the aldehyde derivative (1 equiv, 70 mg), 2,2-difluoro-ethylamine (1.3 equiv, 22 μL) and AcOH (135 μL) in MeOH (4 mL) and DCM (2.7 mL) was stirred at room temperature for 3 h. Na(CN)BH₃ (0.5 equiv, 8 mg) was added and the mixture was stirred at room temperature. After 2 h, the reaction was concentrated and purification by preparative HPLC afforded the desired compound.

### Compound 380: {4-[(2,2-Difluoro-ethylamino)-methyl]-2-ethyl-phenyl}-methyl-(1-ethyl-1H-imidazo[4,5-c]pyridin-6-yl)-amine

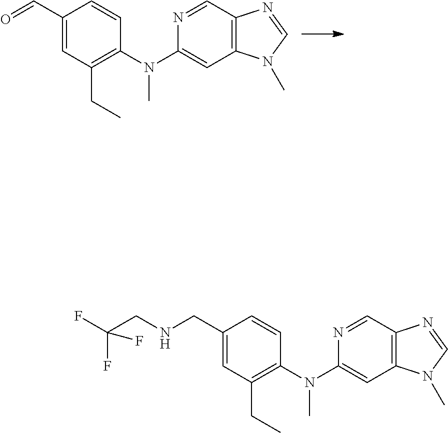 [SMILES: CCc1cc(C=O)ccc1N(C)c1cc2c(cn1)ncn2C ; CCc1cc(CNCC(F)(F)F)ccc1N(C)c1cc2c(cn1)ncn2C]

A solution of the aldehyde derivative (1 equiv, 70 mg), 2,2,2-trifluoro-ethylamine (1.3 equiv, 25 μL) and AcOH (135 μL) in MeOH (4 mL) and DCM (2.7 mL) was stirred at room temperature for 3 h. Na(CN)BH₃ (0.5 equiv, 8 mg) was added and the mixture was stirred at room temperature. After 2 h, the reaction was concentrated and purification by preparative HPLC afforded the desired compound.

### Compound 381: 1-{3-Ethyl-4-[methyl-(1-methyl-1H-imidazo[4,5-c]pyridin-6-yl)-amino]-enzyl}-azetidine-3-carbonitrile

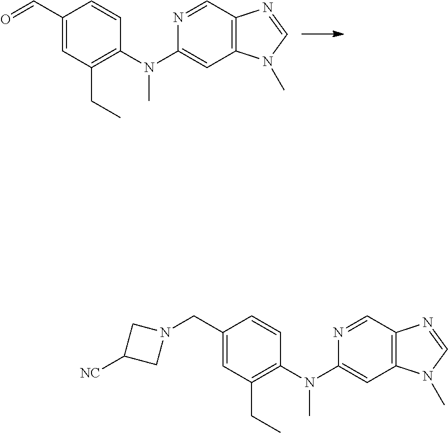 [SMILES: CCc1cc(C=O)ccc1N(C)c1cc2c(cn1)ncn2C ; CCc1cc(CN2CC(C#N)C2)ccc1N(C)c1cc2c(cn1)ncn2C]

A solution of the aldehyde derivative (1 equiv, 110 mg), azetidine-3-carbonitrile (1.3 equiv, 59 mg) and AcOH (150 μL) in MeOH (5.5 mL) and DCM (4.0 mL) was stirred at room temperature for 3 h. Na(CN)BH₃ (0.5 equiv, 12 mg) was added and the mixture was stirred at room temperature. After 2 h, the reaction was concentrated and purification by preparative HPLC afforded the desired compound.

### Compound 382: [4-(3,3-Dimethyl-azetidin-1-ylmethyl)-2-ethyl-phenyl]-methyl-(1-methyl-1H-imidazo[4,5-c]pyridin-6-yl)-amine

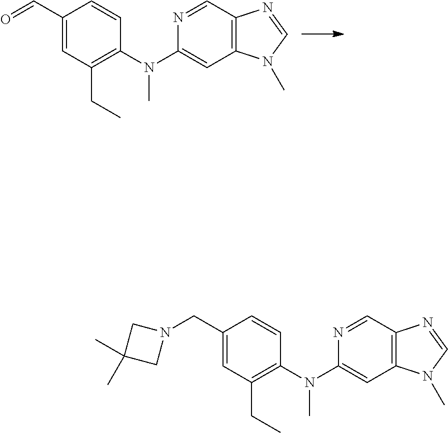 [SMILES: CCc1cc(C=O)ccc1N(C)c1cc2c(cn1)ncn2C ; CCc1cc(CN2CC(C)(C)C2)ccc1N(C)c1cc2c(cn1)ncn2C]

A solution of the aldehyde derivative (1 equiv, 110 mg), 3,3-dimethyl-azetidine (1.3 equiv, 60 mg) and AcOH (150 μL) in MeOH (5.5 mL) and DCM (4.0 mL) was stirred at room temperature for 3 h. Na(CN)BH₃ (0.5 equiv, 12 mg) was added and the mixture was stirred at room temperature. After 2 h, the reaction was concentrated and purification by preparative HPLC afforded the desired compound.

### Compound 383: (4-Ethyl-1′-methanesulfonyl-1′,2′,3′,4′,5′,6′-hexahydro-[2,4′]bipyridinyl-5-yl)-methyl-(1-methyl-1H-imidazo[4,5-c]pyridin-6-yl)-amine

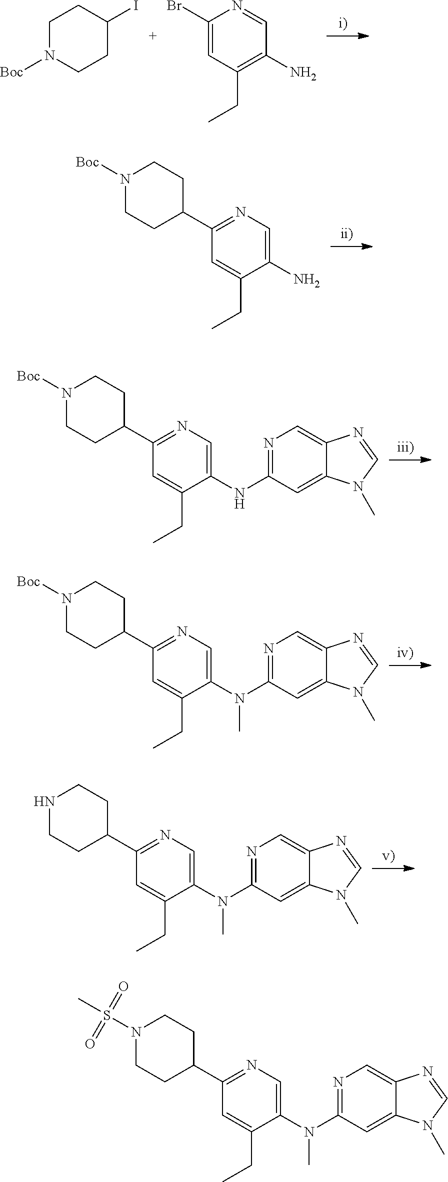

Synthesised following the same conditions used for compound 328 using iodo-piperidine.

### Compound 384: {4-Ethyl-5-(methyl-(1-methyl-1H-imidazo[4,5-c]pyridin-6-yl)-amino)-3′,4′,5′,6′-tetrahydro-2′H-(2,4′]bipyridinyl-1′-yl}-acetonitrile

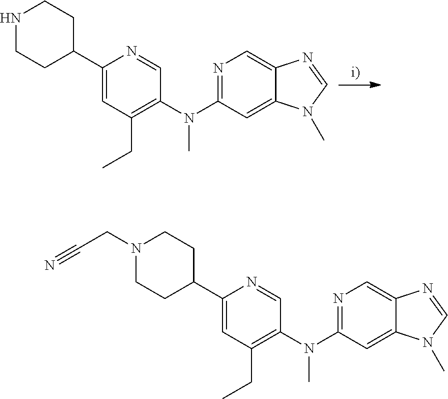 [SMILES: CCc1cc(C2CCNCC2)ncc1N(C)c1cc2c(cn1)ncn2C ; CCc1cc(C2CCN(CC#N)CC2)ncc1N(C)c1cc2c(cn1)ncn2C]

Bromoacetonitrile (1 equiv, 4 μL) was added to a mixture of the piperidine derivative (1 equiv, 20 mg) and K₂CO₃ (2 equiv, 16 mg) in MeCN (0.5 mL) and the reaction was stirred at room temperature for 16 h. The mixture was diluted with EtOAc, washed with water (3×), dried and concentrated. Purification by silica chromatography (NH₃/MeOH/EtOAc; 0.1:1:5) afforded the desired compound.

### Compound 385: 1-{3-Ethyl-4-[methyl-(1-methyl-1H-imidazo[4,5-c]pyridin-6-yl)-amino]-phenyl}-cyclopropanecarbonitrile

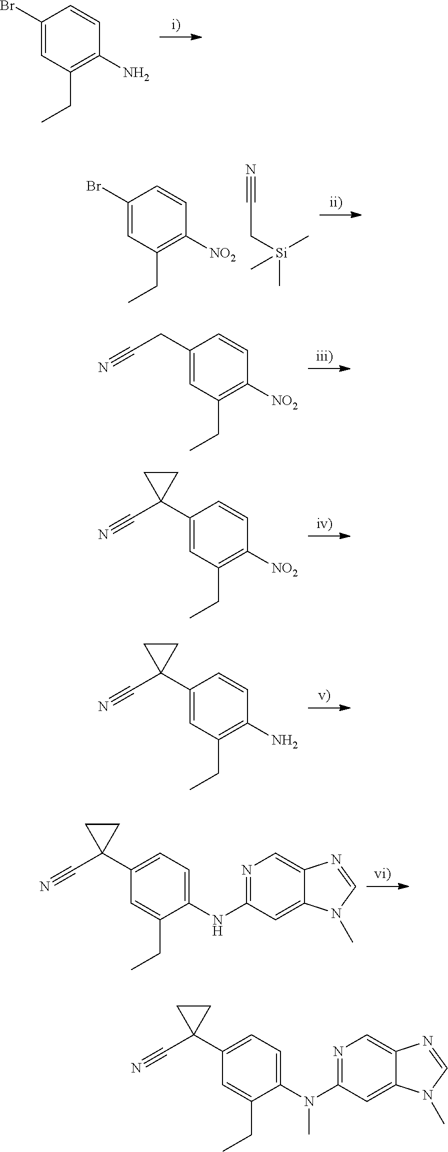

### Step (i): 4-Bromo-2-ethyl-1-nitro-benzene

TFAA (4.8 equiv, 34 mL) was added to a solution of H₂O₂ (4 equiv, 20 mL) in DCM (100 mL) at 0° C. After 5 min, a solution 4-bromo-2-ethyl aniline (1 equiv, 7.1 mL) in DCM (200 mL) was added dropwise over 30 min and the reaction was heated to reflux for 2 h. Aq. sat NaHCO₃ solution was added, organic layer separated, dried and concentrated. The residue was purified by silica chromatography (EtOAc/cyclohexane) to afford the intermediate.

### Step (ii): (3-Ethyl-4-nitro phenyl)-acetonitrile

An extra dry vial charged with xanthphos (0.02 equiv, 151 mg), Pd₂(dba)₃ (0.01 equiv, 119 mg) and the bromoaryl (1 equiv, 3 g) was flushed with argon and dry DMF (13 mL) was added. Trimethylsilyl acetonitrile (1.2 equiv, 2.1 mL) was added, followed by ZnF₂ (0.6 equiv, 809 mg), the mixture was again flushed with argon and the mixture was heated in a microwave at 150° C. for 5 h. The reaction mixture was diluted with EtOAc, washed with water, dried and concentrated. Purification by silica chromatography (EtOAc/cyclohexane; 0:100 to 30:70) afforded the desired product.

### Step (iii): 1-(3-Ethyl-4-nitro-phenyl)-cyclopropanecarbonitrile

Aq. NaOH solution (50%, 1 mL) was added dropwised to a solution of the arylacetonitrile (1 equiv, 500 mg), TBAB (1 equiv, 847 mg) and 1,2-dibromoethane (3 equiv, 0.68 mL) MeCN (5 mL) after what the mixture was heated to 40° C. for 2 h. Water was added and the solution was extracted with DCM (3×), dried and concentrated. Purification by silica chromatography (EtOAc/cyclohexane; 0:100 to 30:70) afforded the desired product.

### Step (iv): 1-(4-Amino-3-ethyl-phenyl)-cyclopropanecarbonitrite

Pd/C (10%, 41 mg) was added to the nitroaryl (1 equiv, 410 mg) in dry methanol (8 mL) and the mixture was stirred under hydrogen atmosphere at room temperature for 3 h. The reaction mixture was filtered through Celite to afford desired product.

### Step (v): 1-[3-Ethyl-4-(1-methyl-1H-imidazo[4,5-c]pyridin-6-ylamino)-phenyl]-cyclopropane carbonitrite

A degassed mixture of the amine derivative (1.1 equiv, 119 mg), 6-chloro-1-methyl-1H-imidazo[4,5-c]pyridine (1 equiv, 98 mg), Cs₂CO₃ (2 equiv, 378 mg), Xphos (0.3 equiv, 83 mg) and palladium(II) acetate (0.1 equiv, 13 mg) in dry toluene (5 mL) was heated at 110° C. for 18 h. The mixture was filtered through Celite and concentrated. The residue was purified by silica chromatography (MeOH/DCM; 0:100 to 10:90) to give the desired compound.

### Step (vi): 1-{3-Ethyl-4-[methyl-(1-methyl-M-imidazo[4,5-c]pyridin-6-yl)-amino]-phenyl}-cyclopropanecarbonitrile

Sodium hydride (1.1 equiv, 8 mg) was added to a solution of the amine derivative (1 equiv, 57 mg) in DMF (3 mL) at 0° C. After 15 min, MeI (1 equiv, 12 μL) was added and resulting mixture was stirred at 0° C. After 4 h, water was added and the reaction mixture extracted with EtOAc (3×), dried and concentrated. The residue was purified by silica chromatography (MeOH/DCM; 0:100 to 10:90) to give the desired compound.

### Compound 386: 1-(3,3-Dimethyl-azetidin-1-yl)-2-{3-ethyl-4-[methyl-(1-methyl-1H-imidazo[4,5-c]pyridin-6-yl)-amino]-phenyl}-ethanone

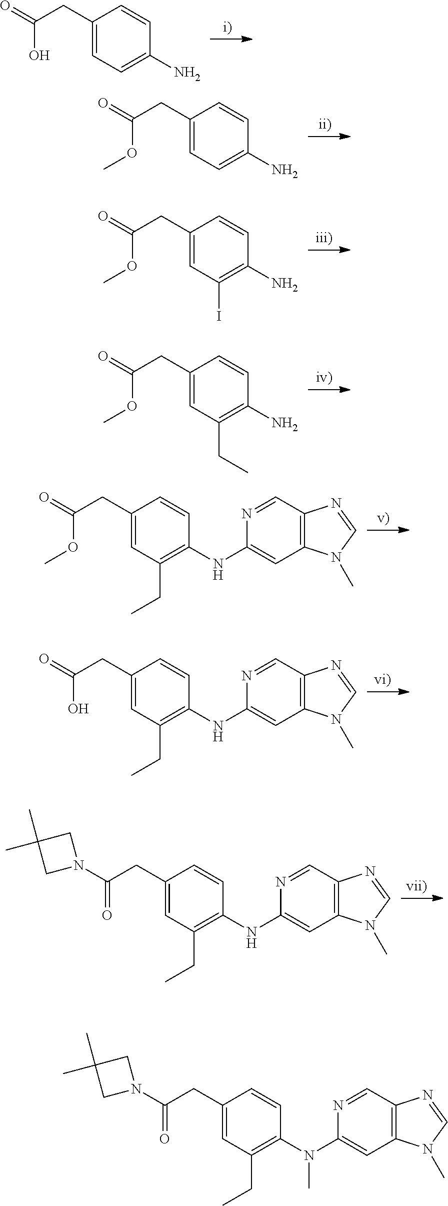

### Step (i): (4-Amino-phenyl)-acetic acid methyl ester

To a solution of 4-aminophenylacetic acid (1 equiv, 10 g) in MeOH (150 mL) was added aq. 37% HCl (25 mL). The mixture was left to stir at room temperature over 3 days. The reaction mixture was then quenched with aq. sat. of NaHCO₃, MeOH was evaporated under reduced pressure and the aqueous layer was extracted with EtOAc (4×). The combined organics were dried and concentrated to give the desired product.

### Step (ii): (4-Amino-3-iodo-phenyl)-acetic acid methyl ester

ICl (1M in DCM, 1.4 equiv, 15 mL) was added to a solution of the ester (1 equiv, 2.0 g) in DCM (200 mL). After 4 h, aq. sat. Na₂S₂O₃ was added and the solution was extracted with DCM (3×). The combined organics were washed with water, brine, dried and concentrated. The residue was purified by silica chromatography (EtOAc/cyclohexane; 3:97 to 30:70) to afford the desired product.

### Step (iii) and (iv): [3-Ethyl-4-(1-methyl-1H-imidazo[4,5-c]pyridin-6-ylamino)-phenyl]-acetic acid methyl ester

Synthesised following the same conditions used for compound 346 (step (ii)) and compound 385 (step (v)).

### Step (v): [3-Ethyl-4-(1-methyl-1H-imidazo[4,5-c]pyridin-6-ylamino)-phenyl]-acetic acid

LiOH (5 equiv, 49 mg) was added to a solution of [3-ethyl-4-(1-methyl-1H-imidazo[4,5-c]pyridin-6-ylamino)-phenyl]-acetic acid methyl ester (1 equiv, 133 mg) in MeOH (2.5 mL) and water (1 mL) and the mixture was heated at 45° C. for 2.5 h. The reaction mixture was concentrated into a smaller volume, water was added and mixture extracted with DCM. The aqueous layer was neutralised with aq. 1N HCl solution and evaporated to dryness.

### Step (vi): 1-(3,3-Dimethyl-azetidin-1-yl)-2-[3-ethyl-4-(1-methyl-1H-imidazo[4,5-c]pyridin-6-ylamino)-phenyl]-ethanone

DIPEA (5 equiv, 0.36 mL) was added to a mixture of the acid (1 equiv, 195 mg), 3,3-dimethyl-azetidine hydrochloride (1.1 equiv, 55 mg), TBTU (1.1 equiv, 145 mg) in dry DMF (2 mL) at 0° C. and the reaction was stirred at room temperature for 3 h. The reaction mixture was diluted with EtOAc, washed with aq. sat. NaHCO₃ solution, water and brine. The organic phase was dried and concentrated. The residue was purified by silica chromatography (MeOH/EtOAc; 0:100 to 20:80) to give the desired compound.

### Step (vii): 1-(3,3-Dimethyl-azetidin-1-yl)-2-{3-ethyl-4-[methyl-(1-methyl-1H-imidazo[4,5-c]pyridin-6-yl)-amino]-phenyl}-ethanone

Synthesised following the same conditions used for compound 385 step (O).

### Compound 387: N-(2-Cyano-1-{3-ethyl-4-[methyl-(1-methyl-1H-imidazo[4,5-c]pyridin-6-yl)-amino]-phenyl}-ethyl)-methanesulfonamide

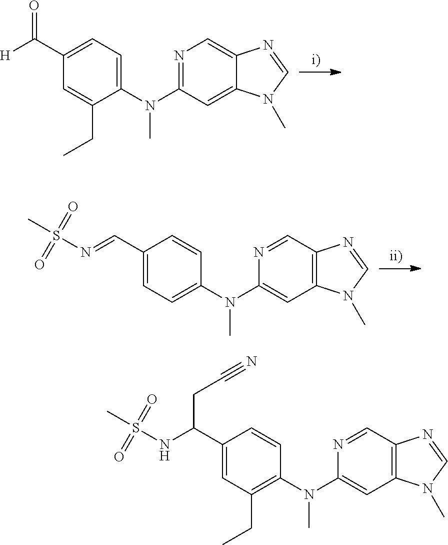

Synthesised following the same conditions used for compound 342.

### Compound 388: N-Cyanomethyl-N-{3-ethyl-4-[methyl-(1-methyl-1H-imidazo[4,5-c]pyridin-6-yl)-amino]-benzyl}-methanesulfonamide

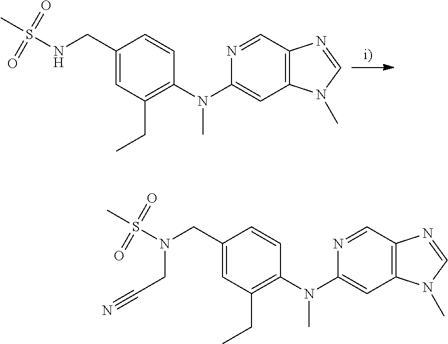 [SMILES: CCc1cc(CNS(C)(=O)=O)ccc1N(C)c1cc2c(cn1)ncn2C ; CCc1cc(CN(CC#N)S(C)(=O)=O)ccc1N(C)c1cc2c(cn1)ncn2C]

Sodium hydride (1 equiv, 3.2 mg) was added to a solution of the sulfonamide (1 equiv, 30 mg) in DMF (0.5 mL) at 0° C. After 15 min, chloroacetonitrile (1 equiv, 5 μL) was added and the solution was stirred at room temperature. After 30 min, water was added and the mixture extracted with DCM, dried and concentrated to give the desired compound.

### Compound 389: (3-{3-Ethyl-4-[methyl-(1-methyl-1H-imidazo[4,5-c]pyridin-6-yl)-amino]-phenyl}-oxetan-3-yl)-acetonitrile

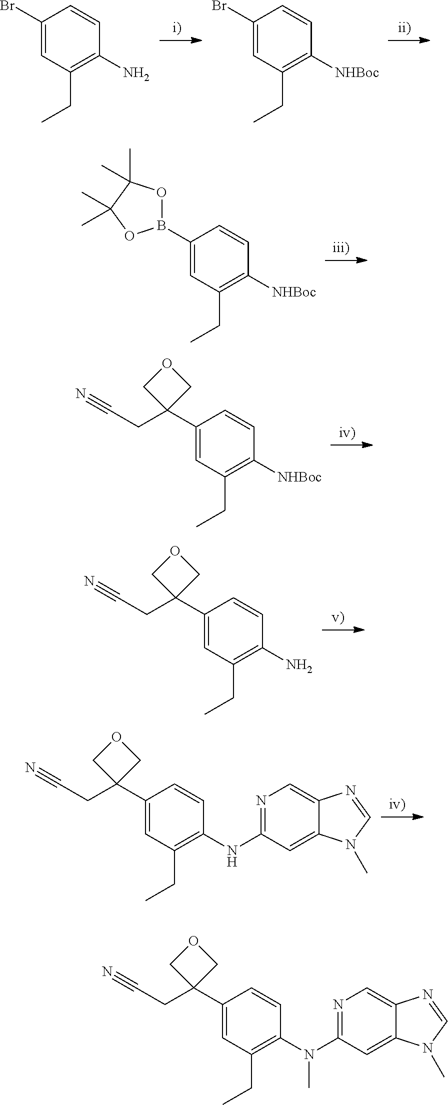

### Step (i): (4-Bromo-2-ethyl-phenyl)-carbamic acid tert-butyl ester

A solution of 4-bromo-2-ethyl aniline (1 equiv, 0.7 mL) and boc-anhydride (1.5 equiv, 1.6 g) in EtOH (7.7 mL) was stirred at room temperature for 18 h. The reaction mixture was concentrated and the residue was dissolved in EtOAc, washed with aq. sat. NaHCO₃ solution, dried and concentrated to give the desired compound.

### Step (ii): [2-Ethyl-4-(4,4,5,5-tetramethyl-[1,3,2]dioxaborolan-2-yl)-phenyl]-carbamic acid tert-butyl ester

To a mixture of the bromo-aryl (1 equiv, 1 g), bis(pinacolato)diboron (1.1 equiv, 0.94 g), KOAc (2.5 equiv, 0.82 g) in dry dioxane (21 mL) was added PdCl₂dppf (0.02 equiv, 55 mg). The mixture was degased under inert atmosphere and heated at 80° C. for 18 h. After filtration through Celite, the mixture was concentrated. Purification by silica chromatography (EtOAc/cyclohexane; 0:100 to 20:80) gave the desired compound.

### Step (iii): [4-(3-Cyanomethyl-oxetan-3-yl)-2-ethyl-phenyl]-carbamic acid tert-butyl ester

2-(Oxetan-3-ylidene)acetonitrile (1 equiv, 50 mg) and the boronic ester derivative (1.5 equiv, 278 mg) were added to a solution of chloro(1,5-cyclooctadiene)rhodium(I) dimer (0.05 equiv, 13 mg) and aq. 1.5M KOH solution (0.46 mL) in 1,4-dioxane (2 mL) and the reaction mixture was stirred at room temperature for 48 h. Brine was added and the mixture extracted with EtOAc (3×). The combined organics were dried and concentrated. Purification by silica chromatography (EtOAc/cyclohexane; 0:100 to 30:70) gave the desired compound.

### Step (iv): [[3-(4-Amino-3-ethyl-phenyl)-oxetan-3-yl]-acetonitrile

TFA (20 equiv, 0.37 mL) was added to a solution of the Boc-protected amine derivative (1 equiv, 77 mg) in dry DCM (2 mL) at 0° C. and the solution was stirred at room temperature for 2 h. Purification by SCX column afforded the product.

### Step (v) and (vi): (3-{3-Ethyl-4-[methyl-(1-methyl-1H-imidazo[4,5-c]pyridin-6-yl)-amino]-phenyl}-oxetan-3-yl)-acetonitrile

Synthesised following the same conditions used for compound 317 (step ii) and compound 385 (step (v)).

### Compound 390: {3-Ethyl-4-[methyl-(1-methyl-1H-imidazo[4,5-c]pyridin-6-yl)-amino]-phenyl}-(4-methanesulfonyl-piperazin-1-yl)-acetonitrile

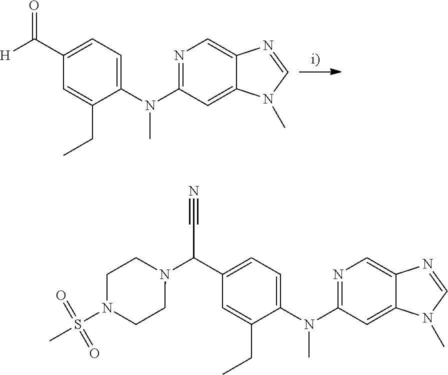 [SMILES: CCc1cc(C=O)ccc1N(C)c1cc2c(cn1)ncn2C ; CCc1cc(C(C#N)N2CCN(S(C)(=O)=O)CC2)ccc1N(C)c1cc2c(cn1)ncn2C]

A mixture of the aldehyde derivative (1 equiv, 45 mg), 1-methanesulfonyl piperazine (1 equiv, 25 mg) and zinc cyanide (1 equiv, 18 mg) in EtOH (0.6 mL) and AcOH (0.3 mL) was irradiated in microwave Biotage Initiator at 100° C. for 30 min. Purification by silica chromatography (MeOH/DCM; 0:100 to 20:80) afforded the desired compound.

### Compound 391: 1-(Cyano-{3-ethyl-4-[methyl-(1-methyl-1H-imidazo[4,5-c]pyridin-6-yl)-amino]-phenyl}-methyl)-azetidine-3-carbonitrile

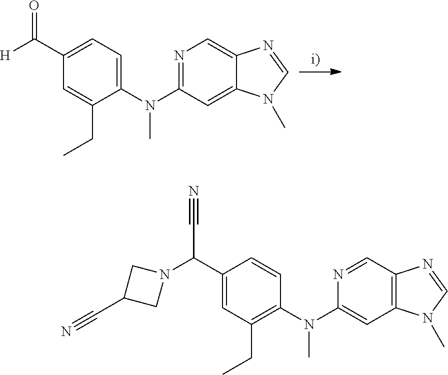 [SMILES: CCc1cc(C=O)ccc1N(C)c1cc2c(cn1)ncn2C ; CCc1cc(C(C#N)N2CC(C#N)C2)ccc1N(C)c1cc2c(cn1)ncn2C]

A mixture of the aldehyde detivative (1 equiv, 50 mg), 3-cyanoazetidine hydrochloride (1 equiv, 20 mg), zinc cyanide (1 equiv, 20 mg) and DIPEA (1 equiv, 30 μL) in EtOH (0.3 mL) and AcOH (0.3 mL) was irradiated in microwave Biotage Initiator at 100° C. for 30 min. Purification by silica chromatography (MeOH/DCM; 0:100 to 20:80) afforded the desired compound.

### Compound 392: N-[2-(3-{3-Ethyl-4-[methyl-(1-methyl-1H-imidazo[4,5-c]pyridin-6-yl)-amino]-phenyl}-oxetan-3-yl)-ethyl]-methanesulfonamide

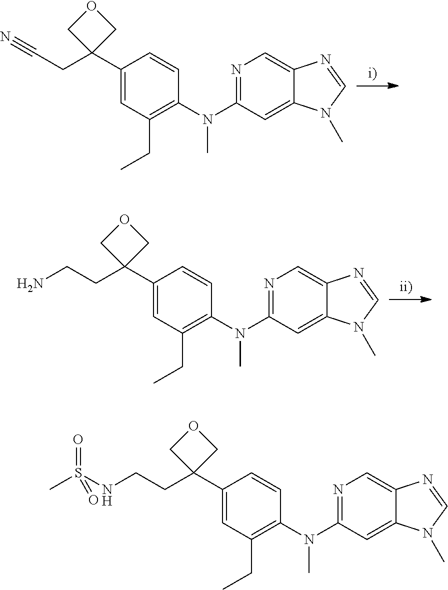 [SMILES: CCc1cc(C2(CC#N)COC2)ccc1N(C)c1cc2c(cn1)ncn2C ; CCc1cc(C2(CCN)COC2)ccc1N(C)c1cc2c(cn1)ncn2C ; CCc1cc(C2(CCNS(C)(=O)=O)COC2)ccc1N(C)c1cc2c(cn1)ncn2C]

### Step (i): {4-[3-(2-Amino-ethyl)-oxetan-3-yl]-2-ethyl-phenyl}-methyl-(1-methyl-1H-imidazo[4,5-c]pyridin-6-yl)-amine

Raney-Nickel (50% in H₂O, 3.4 mL) was added to a solution of the nitrile derivative (1 equiv, 285 mg) in THF (10 mL) and the mixture was stirred under an atmosphere of hydrogen at room temperature for 18 h. The mixture was filtered and concentrated to afford the product.

### Step (ii): N-[2-(3-{3-Ethyl-4-[methyl-(1-methyl-1H-imidazo[4,5-c]pyridin-6-yl)-amino]-phenyl}-oxetan-3-A-ethyl]-methanesulfonamide

Triethylamine (1.1 equiv, 63 μL) was added to a solution of the amine (1 equiv, 150 mg) in DCM (5 mL) at 0° C. After 10 min, methanesulfonyl chloride (1.1 equiv, 35 μL) was added and the mixture was stirred for 1 h. Water was then added and the residual mixture was extracted with DCM (3×), dried and concentrated. Purification by silica chromatography (MeOH/DCM; 0:100 to 10:90) afforded the desired compound.

### Compound 393: {3-Ethyl-4-[methyl-(1-methyl-1H-imidazo[4,5-c]pyridin-6-yl)-amino]-phenyl}-morpholin-4-yl-acetonitrile

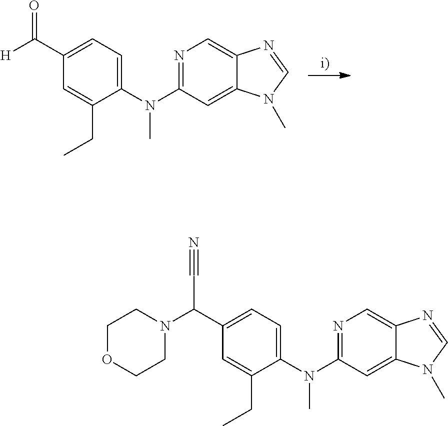 [SMILES: CCc1cc(C=O)ccc1N(C)c1cc2c(cn1)ncn2C ; CCc1cc(C(C#N)N2CCOCC2)ccc1N(C)c1cc2c(cn1)ncn2C]

A mixture of the aldehyde derivative (1 equiv, 25 mg), morpholine (1 equiv, 7 μL) and zinc cyanide (1 equiv, 10 mg) in EtOH (0.6 mL) and AcOH (0.2 mL) was irradiated in microwave Biotage Initiator at 80° C. for 15 min. Water was added and the mixture extracted with DCM, dried and concentrated. Purification by silica chromatography (MeOH/DCM; 0:100 to 4:96) afforded the desired compound.

### Compound 394: {3-Ethyl-4-[methyl-(1-methyl-1H-imidazo[4,5-c]pyridin-6-yl)-amino]-phenyl}-morpholin-4-yl-acetonitrile

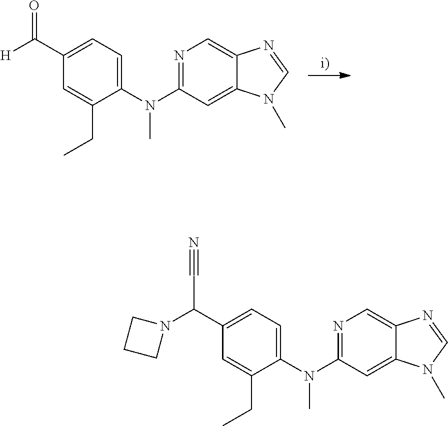 [SMILES: CCc1cc(C=O)ccc1N(C)c1cc2c(cn1)ncn2C ; CCc1cc(C(C#N)N2CCC2)ccc1N(C)c1cc2c(cn1)ncn2C]

A mixture of the aldehyde deivative (1 equiv, 30.6 mg), azetidine (1 equiv, 7 μL) and zinc cyanide (1 equiv, 12.2 mg) in EtOH (0.75 mL) and AcOH (0.25 mL) was irradiated in microwave Biotage Initiator at 80° C. for 15 min. Water was added and the mixture extracted with DCM, dried and concentrated. Purification by silica chromatography (MeOH/DCM; 0:100 to 4:96) afforded the desired compound.

### Compound 395: (4,4-Difluoro-piperidin-1-yl)-{3-ethyl-4-[methyl-(1-methyl-1H-imidazo[4,5-c]pyridin-6-yl)-amino]-phenyl}-acetonitrile

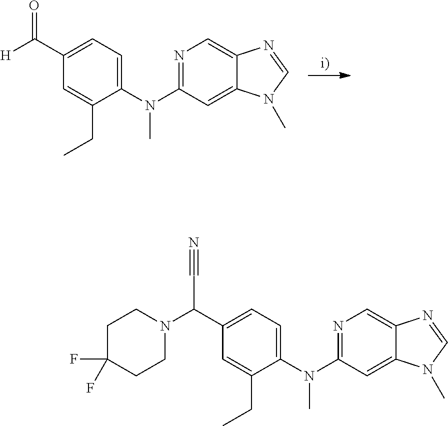 [SMILES: CCc1cc(C=O)ccc1N(C)c1cc2c(cn1)ncn2C ; CCc1cc(C(C#N)N2CCC(F)(F)CC2)ccc1N(C)c1cc2c(cn1)ncn2C]

A mixture of the aldehyde derivative (1 equiv, 50 mg), 4,4-difluoropiperidine hydrochloride (1 equiv, 27 mg), zinc cyanide (1 equiv, 20 mg) and DIPEA (1 equiv, 30 μL) in EtOH (0.3 mL) and AcOH (0.3 mL) was irradiated in microwave Biotage Initiator at 100° C. for 15 min. Purification by silica chromatography (MeOH/DCM; 0:100 to 20:80) afforded the desired compound.

### Compound 396: (4-Acetyl-piperazin-1-yl)-{3-ethyl-4-[methyl-(1-methyl-1H-imidazo[4,5-c]pyridin-6-yl)-amino]phenyl}-acetonitrile

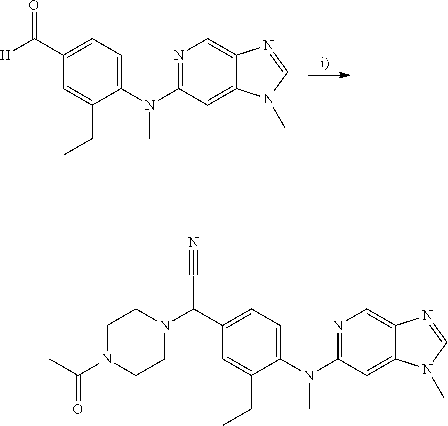 [SMILES: CCc1cc(C=O)ccc1N(C)c1cc2c(cn1)ncn2C ; CCc1cc(C(C#N)N2CCN(C(C)=O)CC2)ccc1N(C)c1cc2c(cn1)ncn2C]

A mixture of the aldehyde derivative (1 equiv, 50 mg), 1-acetylpiperazine (1 equiv, 22 mg) and zinc cyanide (1 equiv, 20 mg) in EtOH (0.6 mL) and AcOH (0.3 mL) was irradiated in microwave Biotage Initiator at 100° C. for 15 min. Purification by silica chromatography (MeOH/DCM; 0:100 to 20:80) afforded the desired compound.

### Compound 397 and 398: N-[2-(3-{3-Ethyl-4-[methyl-(1-methyl-1H-imidazo[4,5-c]pyridin-6-yl)-amino]-phenyl}-oxetan-3-yl)-ethyl]-C,C-difluoro-methanesulfonamide and difluoro-methanesulfonic acid 3-{3-ethyl-4-[methyl-(1-methyl-1H-imidazo[4,5-c]pyridin-6-yl)-amino]-phenyl}-pyrrolidin-3-ylmethyl ester

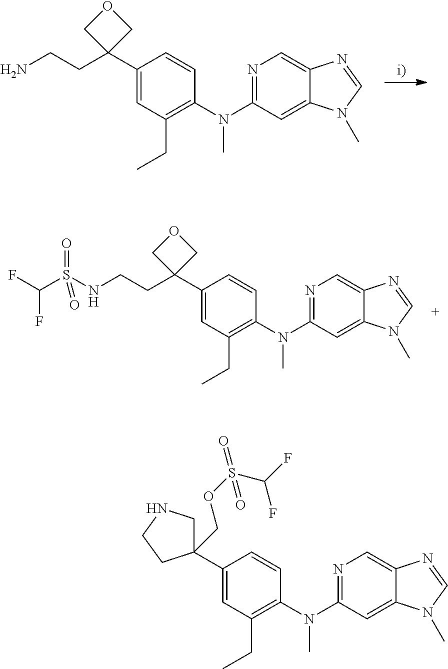 [SMILES: CCc1cc(C2(CCN)COC2)ccc1N(C)c1cc2c(cn1)ncn2C ; CCc1cc(C2(CCNS(=O)(=O)C(F)F)COC2)ccc1N(C)c1cc2c(cn1)ncn2C ; CCc1cc(C2(COS(=O)(=O)C(F)F)CCNC2)ccc1N(C)c1cc2c(cn1)ncn2C]

Pyridine (1.1 equiv, 36 μL) was added to a solution of the amine derivative (1 equiv, 145 mg) in DCM (5 mL) at 0° C. After 10 min, 1,1-difluoromethanesulfonyl chloride (1.1 equiv, 39 μL) was added portionwised and the mixture was stirred for 1.5 h. Water was then added and the residual mixture was extracted with DCM (3×), dried and concentrated. Purification by preparative HPLC afforded the isolation of both compounds.

### Compound 399: 1-{4-Ethyl-5-[methyl-(1-methyl-1H-imidazo[4,5-c]pyridin-6-yl)-amino]-pyridin-2-yl}-azetidine-3-carboxylic acid methyl ester

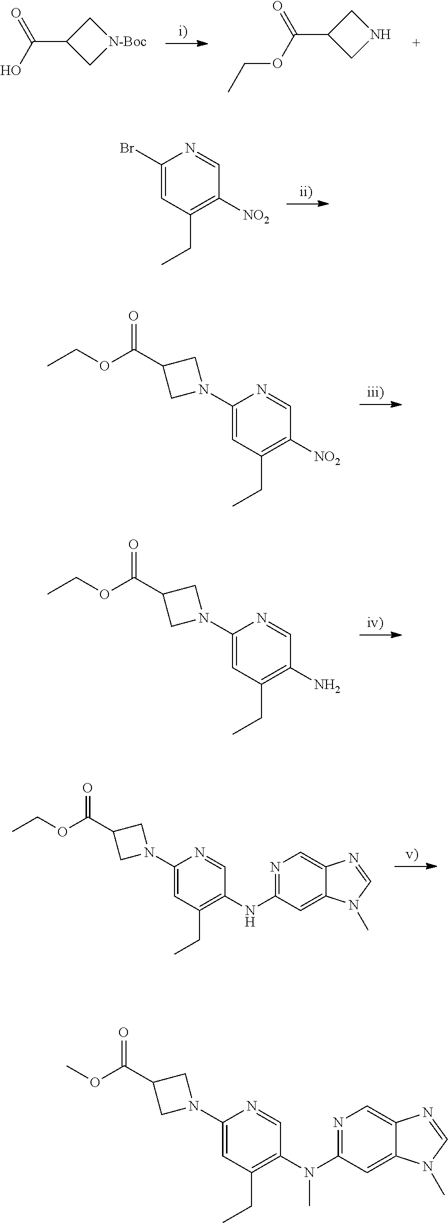

### Step (i): Azetidine-3-carboxylic acid ethyl ester

Thionyl chloride (1.2 equiv, 0.22 mL) was added dropwised to a stirred solution of azetidine-1,3-dicarboxylic acid mono-tert-butyl ester (1 equiv, 500 mg) in EtOH (7 mL) at −10° C. and the reaction mixture was stirred room temperature for 16 h. Concentration afforded the desired compound.

### Step (ii): 1-(4-Ethyl-5-nitro-pyridin-2-yl)-azetidine-3-carboxylic acid ethyl ester

A mixture of 2-bromo-4-ethyl-5-nitro-pyridine (1 equiv, 522 mg), the azetidine (1.1 equiv, 321 mg) and potassium carbonate (2.5 equiv, 859 mg) in dry DMF (4 mL) was stirred and then 3 h at 70° C. Water was added and the aqueous extracted with CH₂Cl₂ (3×). The combined organics were dried and concentrated. Purification by silica chromatography (EtOAc/hexane; 0:100 to 30:70) gave the desired compound.

### Step (iii): 1-(5-Amino-4-ethyl-pyridin-2-yl)-azetidine-3-carboxylic acid ethyl ester

Palladium on carbon (10% wt., 4.5 mg) was added to a solution of the nitroaryl (1 equiv, 30 mg) in EtOH (0.5 mL) and the mixture was stirred under a hydrogen atmosphere at room temperature for 16 h.

The reaction mixture was filtered through Celite and the filtrate was concentrated to afford the desired product.

### Step (iv): 1-[4-Ethyl-5-(1-methyl-1H-imidazo[4,5-c]pyridin-6-ylamino)-pyridin-2-yl]-azetidine-3-carboxylic acid methyl ester

A mixture of the aniline (1.1 equiv, 189 mg), 6-chloro-1-methyl-1H-imidazo[4,5-c]pyridine (1 equiv, 116 mg), BINAP (0.2 equiv, 86 mg), Cs₂CO₃ (3.0 equiv, 675 mg) and tris(dibenzylideneacetone)dipalladium(0) (0.1 equiv, 63 mg) in dry 1,4-dioxane (2.4 mL), degassed under (N₂), was heated at 110° C. for 16 h. The reaction mixture was filtered, and the residue washed with MeOH. The combined filtrates were concentrated, water was added and the mixture washed with CH₂Cl₂. The aqueous layer was evaporated to dryness under reduced pressure to yield the acid form that was used in the next step without further purification The residue was dissolved in MeOH (10 mL), HCl (1 mL, 4 M in 1,4-dioxane) was added and the reaction was heated at 68° C. for 1 h. The reaction mixture was concentrated, diluted with EtOAc and washed with aq. sat. NaHCO₃ and brine. The combined organics was dried and concentrated to afford the product.

### Step (v): 1-[4-Ethyl-5-(1-methyl-1H-imidazo[4,5-c]pyridin-6-ylamino)-pyridin-2-yl]-azetidine-3-carboxylic acid ethyl ester

Synthesised following the same conditions used for compound 385 (step (v)).

### Compound 400:1-{4-Ethyl-5-[methyl-(1-methyl-1H-imidazo[4,5-c]pyridin-6-yl)-amino]-pyridin-2-yl}-azetidine-3-carboxylic acid methylamide

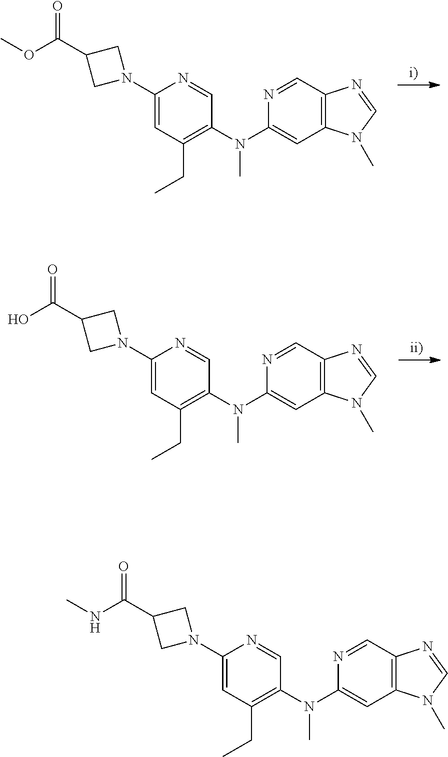 [SMILES: CCc1cc(N2CC(C(=O)OC)C2)ncc1N(C)c1cc2c(cn1)ncn2C ; CCc1cc(N2CC(C(=O)O)C2)ncc1N(C)c1cc2c(cn1)ncn2C ; CCc1cc(N2CC(C(=O)NC)C2)ncc1N(C)c1cc2c(cn1)ncn2C]

### Step (i): 1-{4-Ethyl-5-[methyl-(1-methyl-1H-imidazo[4,5-c]pyridin-6-yl)-amino]-pyridin-2-yl}-azetidine-3-carboxylic acid

Aq. 0.1 M NaOH (2 equiv, 3.0 mL) was added to a solution of the methyl ester derivative (1 equiv, 57 mg) in tert-BuOH (3.0 mL) and the reaction mixture was stirred at room temperature for 15 min. Reaction mixture was concentrated, pH adjusted to 5 with aq. 1 N HCl and the mixture concentrated.

### Step (ii): 1-{4-Ethyl-5-[methyl-(1-methyl-1H-imidazo[4,5-c]pyridin-6-yl)-amino]-pyridin-2-yl}-azetidine-3-carboxylic acid methylamide

A mixture of PS-Carbodiimide resin (1.3 equiv, 146 mg), the obtained carboxylic acid derivative (1 equiv, 55 mg) and HOBt (0.7 equiv, 16 mg) in DCM (2 mL) and DMF (0.2 mL) was stirred at room temperature for 5 min followed by the addition of methylamine hydrochloride (1.2 equiv, 12 mg) in DCM (2 mL) and DIPEA (1.2 equiv, 31 μL). The reaction mixture was stirred at room temperature for 18 h. The mixture was filtered and was with DCM and MeOH and the filtered was concentrated. The residue was redissolved in DCM and washed with water, aq. sat. NaHCO₃, dried and concentrated. Purification by silica chromatography (7 N NH₃ solution in MeOH/MeOH/DCM; 0/0/100 to 1/9/90) afforded the desired compound.

### Compound 401: 1-{4-Ethyl-5-[methyl-(1-methyl-1H-imidazo[4,5-c]pyridin-6-yl)-amino]-pyridin-2-yl}-azetidine-3-carboxylic acid cyanomethyl-amide

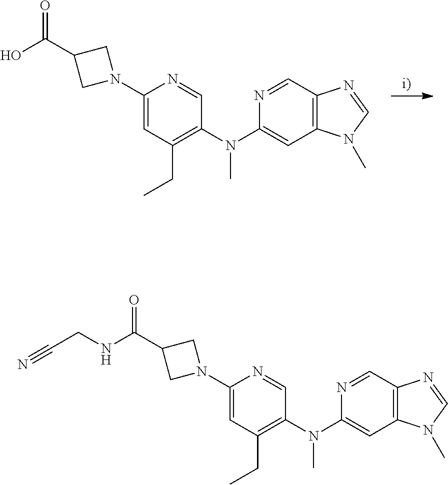 [SMILES: CCc1cc(N2CC(C(=O)O)C2)ncc1N(C)c1cc2c(cn1)ncn2C ; CCc1cc(N2CC(C(=O)NCC#N)C2)ncc1N(C)c1cc2c(cn1)ncn2C]

A mixture of PS-Carbodiimide resin (1.3 equiv, 80 mg), the carboxylic acid derivative (1 equiv, 30 mg) and HOBt (0.7 equiv, 8.8 mg) in DCM (1 mL) and DMF (0.1 mL) was stirred at room temperature for 5 min followed by the addition of aminoacetonitrile hydrochloride (1.2 equiv, 9.1 mg) in DCM (1 mL) and DIPEA (1.2 equiv, 17 μL). The reaction mixture was stirred at 40° C. for 3 days. The mixture was filtered and was with DCM and MeOH and the filtrated was concentrated. The residue was redissolved in DCM and washed with water, aq. sat. NaHCO₃, dried and concentrated. Purification by silica chromatography (7 N NH₃ solution in MeOH/MeOH/DCM; 0/0/100 to 1/9/90) afforded the desired compound.

### Compound 402: 1-{4-Ethyl-5-[methyl-(1-methyl-1H-imidazo[4,5-c]pyridin-6-yl)-amino]-pyridin-2-yl}-azetidine-3-carbonitrile

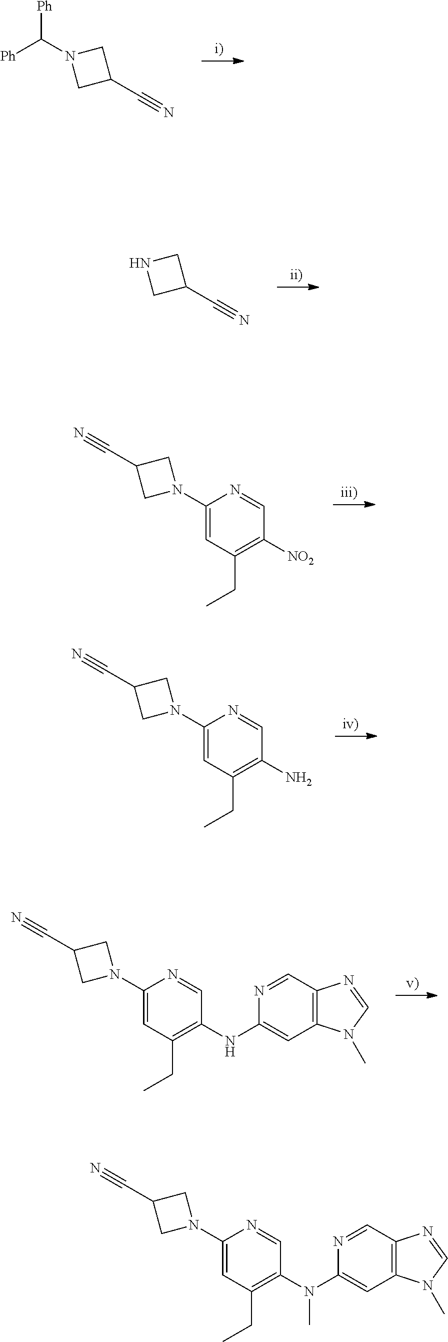 [SMILES: N#CC1CN(C(c2ccccc2)c2ccccc2)C1 ; N#CC1CNC1 ; CCc1cc(N2CC(C#N)C2)ncc1[N+](=O)[O-] ; CCc1cc(N2CC(C#N)C2)ncc1N ; CCc1cc(N2CC(C#N)C2)ncc1Nc1cc2c(cn1)ncn2C ; CCc1cc(N2CC(C#N)C2)ncc1N(C)c1cc2c(cn1)ncn2C]

### Step (i): Azetidine-3-carbonitrile

1-Chloroethyl chloroformate (2 equiv, 0.87 mL) was added to a solution of 1-benzhydryl-azetidine-3-carbonitrile (1 equiv, 1 g) in dry MeCN (88 mL) and the mixture was refluxed for 3 h afterwhat the mixture was concentrated. The residue was dissolved in MeOH (130 mL) and the mixture was refluxed for another 1 h. The solvent was evaporated to give the crude material that was used in the next step without further purification.

### Step (ii): 1-(4-Ethyl-5-nitro-pyridin-2-yl)-azetidine-3-carbonitrile

Synthesised following the same conditions used for compound 399 (step (O).

### Step (iii): 1-(5-Amino-4-ethyl-pyridin-2-yl)-azetidine-3-carbonitrile

Palladium on carbon (10% wt., 0.05 equiv, 15 mg) was added to a solution of the nitroaryl (1 equiv, 65 mg) in dry dioxane (1.7 mL) and the mixture was stirred under a hydrogen atmosphere at room temperature for 6 h. The reaction mixture was filtered through Celite and the filtrate was concentrated to afford the desired product.

### Step (iv) and (v): 1-{4-Ethyl-5-[methyl-(1-methyl-1H-imidazo[4,5-c]pyridin-6-yl)-amino]-pyridin-2-yl}-azetidine-3-carbonitrile

Synthesised following the same conditions used for compound 328 (step (ii)) and compound 385 (step (v)).

### Compound 403: 3,5-Difluoro-4-[methyl-(1-methyl-1H-imidazo[4,5-c]pyridin-6-yl)-amino]-benzonitrile

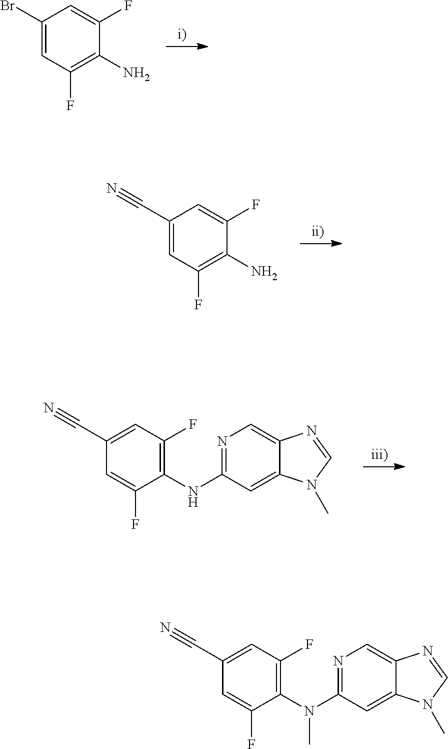 [SMILES: Nc1c(F)cc(Br)cc1F ; N#Cc1cc(F)c(N)c(F)c1 ; Cn1cnc2cnc(Nc3c(F)cc(C#N)cc3F)cc21 ; CN(c1cc2c(cn1)ncn2C)c1c(F)cc(C#N)cc1F]

### Step (i): 4-Amino-3,5-difluoro-benzonitrile

A solution of 4-bromo-2,6-difluorophenylamine (1 equiv, 1 g) and Zn(CN)₂ (0.75 equiv, 423 mg) in dry DMF (10.5 mL) and DMA (3.7 mL) was stirred under argon atmosphere for 10 min and heated up to 90° C. before Pd(PPh₃)₄ (0.15 equiv, 833 mg) was added and the reaction mixture was stirred at 90° C. for 18 h. The resulting mixture was diluted with EtOAc (3×) and washed with brine. The combined organics was dried and concentrate. Purification by silica chromatography (EtOAc/cyclohexane; 0/100 to 25/75) afforded the desired product.

### Step (i)i and (iii): 3,5-Difluoro-4-[methyl-(1-methyl-1H-imidazo[4,5-c]pyridin-6-yl)-amino]-benzonitrile

Synthesised following the same conditions used for compound 328 (step (ii)) and compound 385 (step (v)).

### Compound 404: N-{3,5-Difluoro-4[methyl-(1-methyl-1H-imidazo[4,5-c]pyridin-6-yl)-amino]-benzyl}-methanesulfonamide

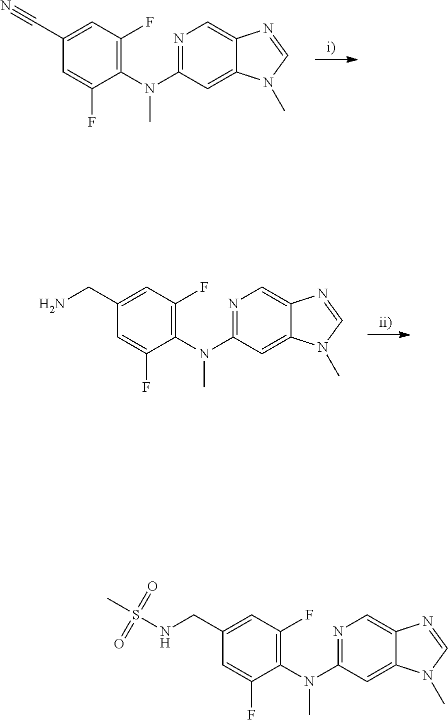 [SMILES: CN(c1cc2c(cn1)ncn2C)c1c(F)cc(C#N)cc1F ; CN(c1cc2c(cn1)ncn2C)c1c(F)cc(CN)cc1F ; CN(c1cc2c(cn1)ncn2C)c1c(F)cc(CNS(C)(=O)=O)cc1F]

### Step (i): (4-Aminomethyl-2,6-difluoro-phenyl)-methyl-(1-methyl-1H-imidazo[4,5-c]pyridin-6-yl)-amine

NiCl₂*6H₂O (1 equiv, 48 mg) and TFA (8 equiv, 124 μL) were added to a solution the nitrile (1 equiv, 60 mg) in dry MeOH (5.5 mL) at 0° C. followed by the slow addition of NaBH₄ (6 equiv, 46 mg). The reaction mixture was stirred at 0° C. for 1 h and then at room temperature for 1 h afterwhat the reaction mixture was loaded onto an SCX coloumn, eluted first with MeOH and then with 2M NH₃ in MeOH to isolate the desired compound.

### Step (ii): N-{3,5-Difluoro-4-[methyl-(1-methyl-1H-imidazo[4,5-c]pyridin-6-yl)-amino]-benzyl}-methanesulfonamide

Triethylamine (3.0 equiv, 77 μL) was added to a solution of the benzylamine (1.0 equiv, 56 mg) in DCM (2 mL) at 0° C. After 15 min, methylsulfonyl chloride (1.1 equiv, 16 μL) was added and the reaction mixture was stirred at room temperature for 3 h. The mixture was diluted with DCM and washed with water. The organic layer was dried and concentrated to afford the desired compound.

### Compound 405: {3,5-Difluoro-4-[methyl-(1-methyl-1H-imidazo[4,5-c]pyridin-6-yl)-amino]-phenyl}-acetonitrile

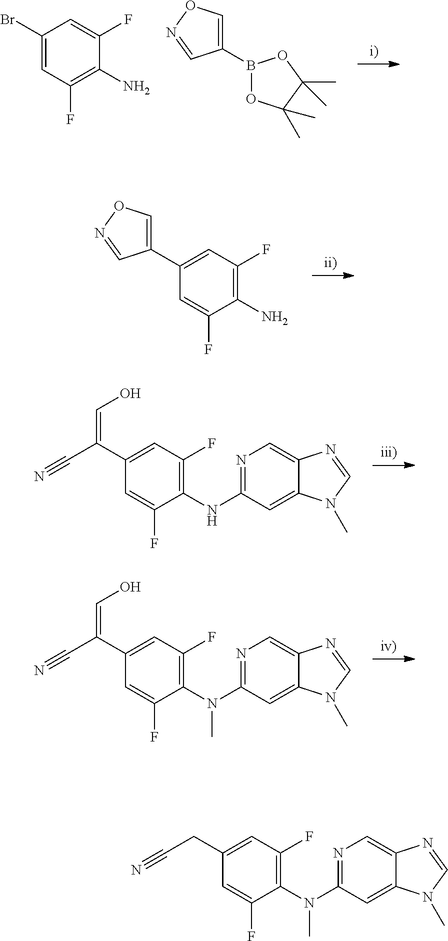

### Step (i): 2,6-Difluoro-4-isoxazol-4-yl-phenylamine

Potassium fluoride (3 equiv, 0.83 g) in water (1 mL) was added to a mixture of 4-isoxazoleboronic acid pinacol ester (1.1 equiv, 1.03 g) and 4-bromo-2,6-difluoroaniline (1.0 equiv, 1 g) in DMF (5 mL), followed by the addition of PdCl₂(dppf) (0.1 equiv, 351 mg). The reaction was stirred at 50° C. for 2 h. The reaction mixture was diluted with EtOAc, washed with water, dried and concentrated. Purification by silica chromatography (EtOAc/cyclohexane; 0:100 to 20:80) gave the desired compound.

### Step (ii)): (E)-2-[3,5-Difluoro-4-(1-methyl-1H-imidazo[4,5-c]pyridin-6-ylamino)-phenyl]-3-hydroxy-acrylonitrile

The aniline derivative (1.0 equiv, 91 mg), 6-chloro-1-methyl-1H-imidazo[4,5-c]pyridine (1 equiv, 78 mg), 2,2′-bis(diphenylphosphino)-1,1′-binaphthyl (0.3 equiv, 87 mg) and Cs₂CO₃ (2.5 equiv, 303 mg) were stirred in DMF (1 mL) and degased under nitrogen followed by the addition of tris(dibenzylideneacetone)-dipalladium(0) (0.1 equiv, 42 mg). The reaction mixture was heated at 100° C. for 18 h after what it was filtered through Celite, washed with MeOH and concentrated. The compound was used as such in the next step.

### Step (iii) and (iv): {3,5-Difluoro-4-[methyl-(1-methyl-1H-imidazo[4,5-c]pyridin-6-yl)-amino]-phenyl}-acetonitrile

Sodium hydride (2.0 equiv, 37 mg) was added to a solution of the amine in dry DMF (1 mL) at 0° C. After 30 min, MeI (1.5 equiv, 39 μL) was added and the solution was stirred at room temperature for 72 h. The solution was concentrated, water was added and the solution neutralized with 1N HCl solution. Used as such in the next step. MeOH (7 mL) was added to this solution and the resulting mixture was heated at 130° C. in a Biotage microwave initiator for 30 min. The methanol was removed in vacuo and the mixture was extracted with EtOAc (3×). The combined organics were dried and concentrated. Purification by silica chromatography (MeOH/EtOAc; 0:100 to 20:80) afforded the desired compound.

### Compound 406: {3-Ethyl-4-[methyl-(1-methyl-1H-imidazo[4,5-c]pyridin-6-yl)-amino]-phenyl}-acetonitrile

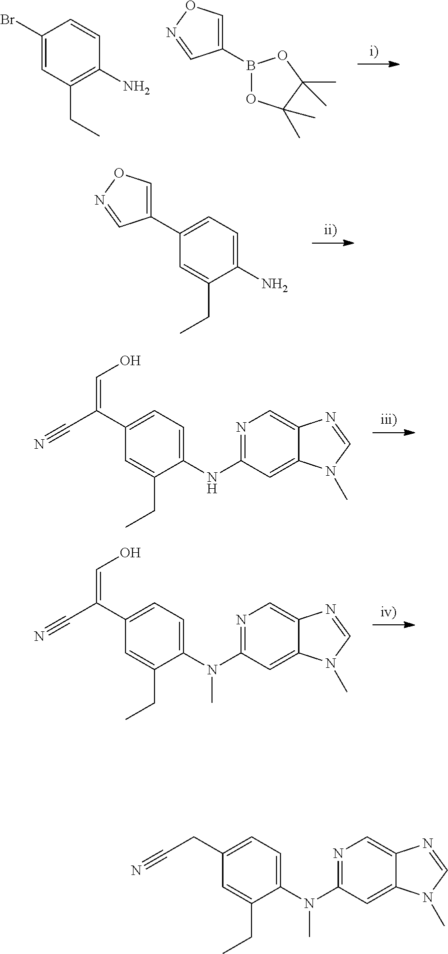

### Step (i) and (ii): (E)-2-[3-Ethyl-4-(1-methyl-1H-imidazo[4,5-c]pyridin-6-ylamino)-phenyl]-3-hydroxy-acrylonitrile

Synthesised following the same conditions used for compound 405 (step (i) and (O).

### Step (iii): (E)-2-{4-Ethyl-5-[methyl-(1-methyl-1H-imidazo[4,5-c]pyridin-6-yl)-amino]-pyridin-2-yl}-3-hydroxy-acrylonitrile

Sodium hydride (2.0 equiv, 5.5 mg) was added to a solution of the amine derivative (1 equiv, 40 mg) in dry DMF (0.7 mL) at 0° C. After 30 min, MeI (1.1 equiv, 9 μL) was added and the solution was stirred for 1 h. Water was added and the mixture extracted with EtOAc (3×), dried and concentrated. Silica chromatography (MeOH/EtOAc; 5:95 to 50:50) afforded the desired compound.

### Step (iv): {3-Ethyl-4-[methyl-(1-methyl-1H-imidazo[4,5-c]pyridin-6-yl)-amino]-phenyl}-acetonitrile

The previous compound (1 equiv, 10 mg) in MeOH (1.3 mL) and water (0.5 mL) was heated at 140° C. for 30 min in a microwave reactor after what the reaction mixture was concentrated and purified by preparative HPLC to afford the desired product.

### Compound 407: {4-Ethyl-5-[methyl-(1-methyl-1H-imidazo[4,5-c]pyridin-6-yl)-amino]-pyridin-2-yl}-acetonitrile

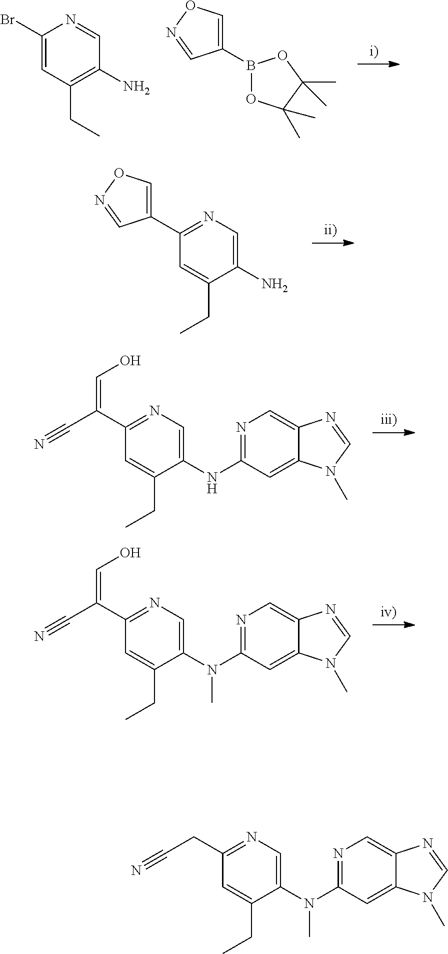

### Step (i) and (ii): (E)-2-[3-Ethyl-4-(1-methyl-1H-imidazo[4,5-c]pyridin-6-ylamino)-phenyl]-3-hydroxy-acrylonitrile

Synthesised following the same conditions used for compound 405 (step (i) and (O).

### Step (iii) and (iv): {3-Ethyl-4-[methyl-(1-methyl-1H-imidazo[4,5-c]pyridin-6-yl)-amino]-phenyl}-acetonitrile

Sodium hydride (3.6 equiv, 16.4 mg) was added to a solution of the amine (1 equiv, 36 mg) in dry DMF (1 mL) at 0° C. After 30 min, MeI (2.0 equiv, 14 μL) was added and the solution was stirred at 5° C. for 18 h. Water (2 mL) was added and washed with EtOAc. To the aqueous layer, MeOH (2 mL) was added and this solution was heated at 90° C. for 3 h. The methanol was removed in vacuo and the mixture was extracted with EtOAc (3×). The combined organics were dried and concentrated. Purification by silica chromatography (MeOH/EtOAc; 0:100 to 10:90) afforded the desired compound

### Compound 408: 3-{3,5-Difluoro-4-[methyl-(1-methyl-1H-imidazo[4,5-c]pyridin-6-yl)-amino]-phenyl)-azetidine-1-carboxylic acid tert-butyl ester

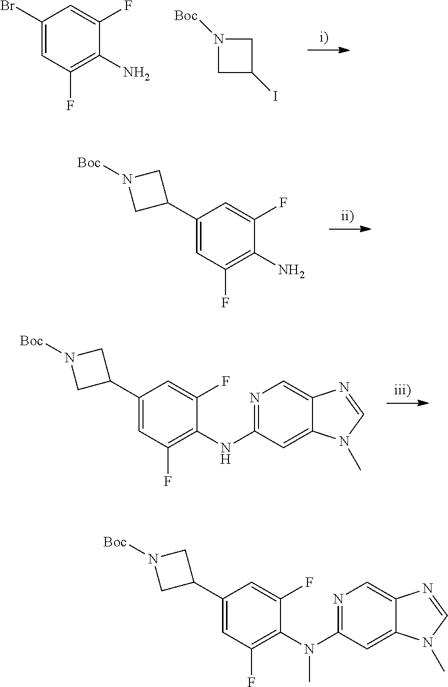 [SMILES: Nc1c(F)cc(Br)cc1F ; CC(C)(C)OC(=O)N1CC(I)C1 ; CC(C)(C)OC(=O)N1CC(c2cc(F)c(N)c(F)c2)C1 ; Cn1cnc2cnc(Nc3c(F)cc(C4CN(C(=O)OC(C)(C)C)C4)cc3F)cc21 ; CN(c1cc2c(cn1)ncn2C)c1c(F)cc(C2CN(C(=O)OC(C)(C)C)C2)cc1F]

### Step (i): 3-(4-Amino-3,5-difluoro-phenyl}-azetidine-1-carboxylic acid tert-butyl ester

N-Boc-azetidine-iodide (1.5 equiv, 4.1 g) was dissolved in DMA (10 mL) and heated at 65° C. Under atmosphere of nitrogen, Rieke zinc (5 g/100 mL, 2 equiv, 25 mL) was added dropwise over 20 min and stirred for 20 min at 65° C. under nitrogen. In another flask 4-bromo-2,6-difluoro-phenylamine (1 equiv, 2.0 g), copper(I) iodide (0.06 equiv, 113 mg) and Pd(dppf)Cl₂ (0.03 equiv, 220 mg) were dissolved in DMA (10 mL) and heated at 85° C. under atmosphere of nitrogen. The reaction mixture from the first flask was added via a cannule to the second flask over 10 min. The resulting reaction mixture was stirred at 85° C. for 3 h. The reactionwass then quenched with aq. sat. NH₄Cl and extracted with EtOAc (3×200 mL). The combined organics was dried and concentrated under reduced pressure. The residue was purified by column chromatography (EtOAc/petroleum ether 40-60; 0:100 to 100:0) to give the expected aniline.

### Step (ii) and (iii): 3-{3,5-Difluoro-4-[methyl-(1-methyl-1H-imidazo[4,5-c]pyridin-6-yl)-amino]-phenyl}-azetidine-1-carboxylic acid tert-butyl ester

Synthesised following the same conditions used for compound 328 (step (ii)) and compound 385 (step (v)).

### Compound 409: [2,6-Difluoro-4-(1-methanesulfonyl-azetidin-3-yl)-phenyl]-methyl-(1-methyl-1H-imidazo[4,5-c]pyridin-6-yl)-amine

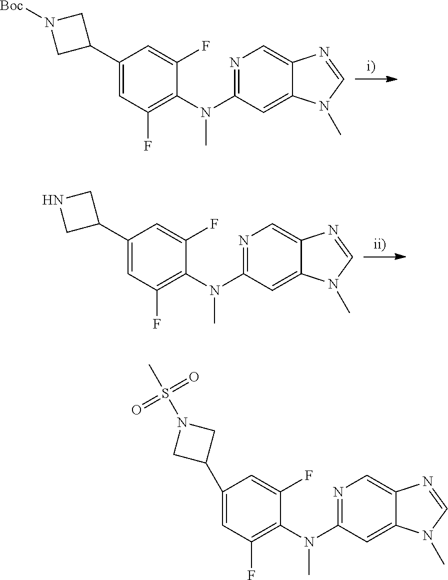

### Step (i): 3 (4-Azetidin-3-yl-2,6-difluoro-phenyl)-methyl-(1-methyl-1H-imidazo[4,5-c]pyridin-6-yl)-amine

HCl (4M solution in 1,4-dioxane, 20 equiv, 2.17 mL) was added to the boc-protected azetidine derivative (1 equiv, 170 mg) in DCM (3 mL). After 2 h, the reaction mixture was concentrated and the product purified by SCX column.

### Step (ii): [2,6-Difluoro-4-(1-methanesulfonyl-azetidin-3-yl)-phenyl]-methyl-(1-methyl-1H-imidazo[4,5-c]pyridin-6-yl)-amine

Pyridine (5 equiv, 34 μL) was added to the azetidine (1 equiv, 28 mg) in DCM (1 mL) followed by the addition of methanesulfonyl chloride (1.20 equiv, 7.9 μL). After stirred for 2 h at room temperature, water was added and the mixture was extracted with DCM (3×), dried and concentrated. Purification by preparative HPLC afforded the desired compound.

### Compound 410: {3-Ethyl-4-[methyl-(1-methyl-1H-imidazo[4,5-c]pyridin-6-yl)-amino]-phenyl}-(3-oxo-piperazin-1-yl)-acetonitrile

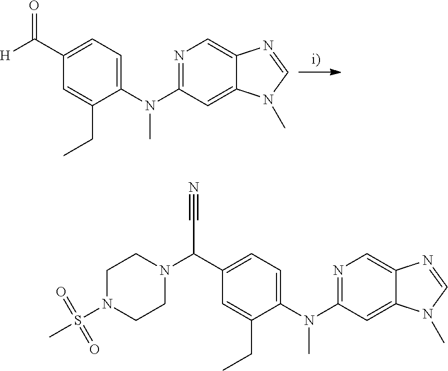 [SMILES: CCc1cc(C=O)ccc1N(C)c1cc2c(cn1)ncn2C ; CCc1cc(C(C#N)N2CCN(S(C)(=O)=O)CC2)ccc1N(C)c1cc2c(cn1)ncn2C]

A mixture of the aldehyde derivative (1 equiv, 80 mg), piperatin-2-one (3 equiv, 82 mg) and zinc cyanide (3 equiv, 96 mg) in EtOH (1.6 mL) and AcOH (0.8 mL) was irradiated in microwave Biotage Initiator at 80° C. for 15 min. The reaction was quenched with water, extracted with DCM (3×), dried and concentrated. Purification by silica chromatography (MeOH/DCM; 0:100 to 10:90) afforded the desired compound.

### Compound 411: [6-(3,3-Difluoro-pyrrolidin-1-yl)-4-ethyl-pyridin-3-yl]-methyl-(1-methyl-1H-imidazo[4,5-c]pyridin-6-yl)-amine

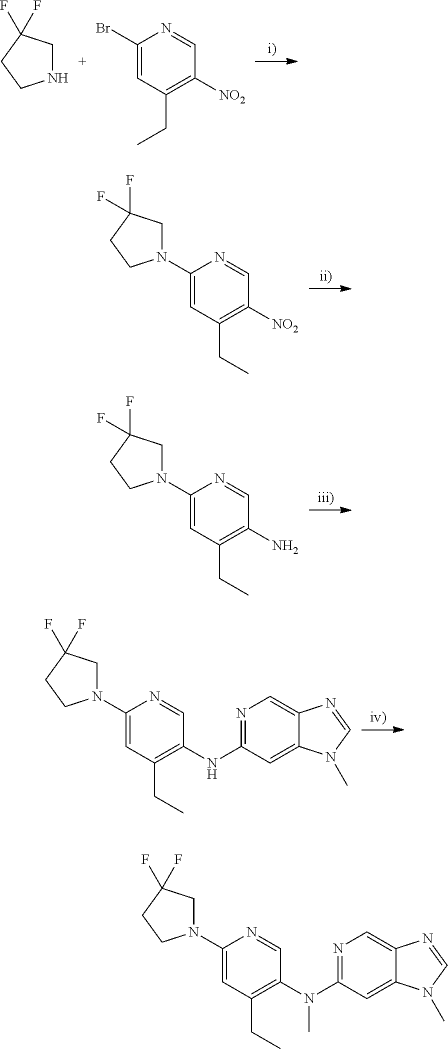

Synthesised following the same conditions used for compound 399.

### Compound 412: 1-{4-Ethyl-5-[methyl-(1-methyl-1H-imidazo[4,5-c]pyridin-6-yl)-amino]-pyridin-2-yl}-azetidine-3-carboxylic acid ethylamide

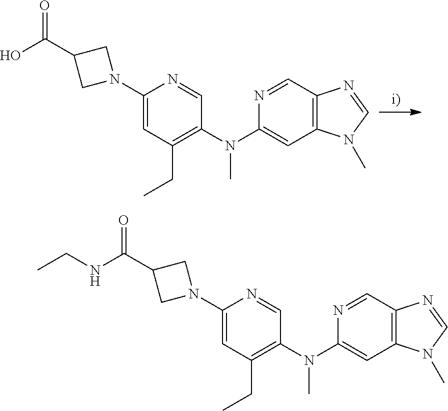

A mixture of PS-Carbodiimide resin (1.3 equiv, 147 mg), the carboxylic acid (1 equiv, 55 mg) and HOBt (0.7 equiv, 16 mg) in DCM (3.7 mL) and DMF (0.2 mL) was stirred at room temperature for 5 min followed by the addition of ethylamine (2M solution in THF, 2.0 equiv, 0.15 mL). The reaction mixture was stirred at room temperature for 18 h. The mixture was filtered and washed with DCM and MeOH and the filtrated was concentrated. The residue was redissolved in DCM and washed with water, aq. sat. NaHCO₃ solution, dried and concentrated. Purification by silica chromatography (7 N NH₃ solution in MeOH/MeOH/DCM; 0:0:100 to 1:9:90) afforded the desired compound.

### Compound 413: [6-(3,3-Difluoro-azetidin-1-yl)-4-ethyl-pyridin-3-yl]-methyl-(1-methyl-1H-imidazo[4,5-c]pyridin-6-yl)-amine

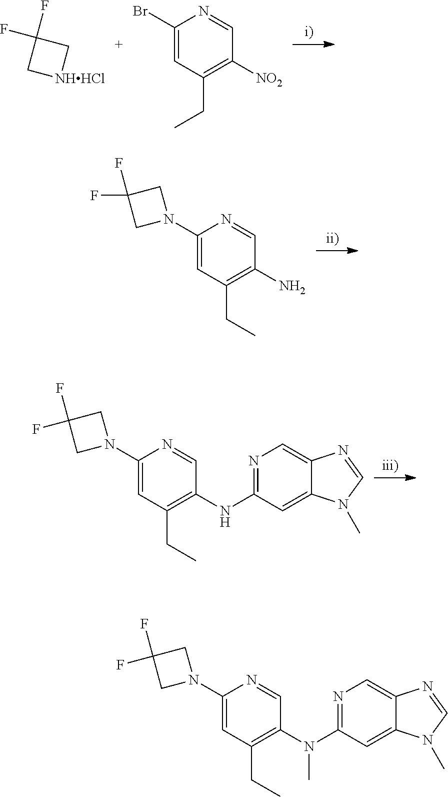

Synthesised following the same conditions used for compound 399.

### Compound 414: [4-Ethyl-6-((R)-3-fluoro-pyrrolidin-1-yl)-pyridin-3-yl]-methyl-(1-methyl-1H-imidazo[4,5-c]pyridin-6-yl)-amine

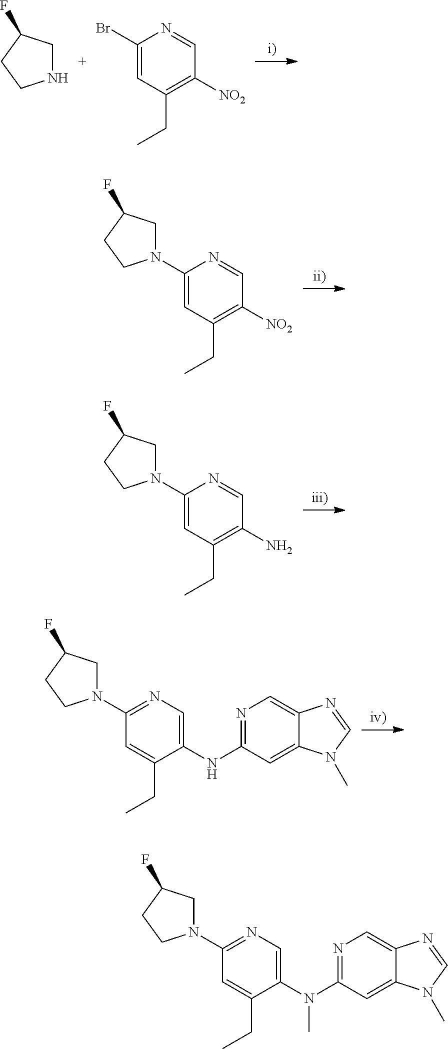 [SMILES: F[C@@H]1CCNC1 ; CCc1cc(Br)ncc1[N+](=O)[O-] ; CCc1cc(N2CC[C@@H](F)C2)ncc1[N+](=O)[O-] ; CCc1cc(N2CC[C@@H](F)C2)ncc1N ; CCc1cc(N2CC[C@@H](F)C2)ncc1Nc1cc2c(cn1)ncn2C ; CCc1cc(N2CC[C@@H](F)C2)ncc1N(C)c1cc2c(cn1)ncn2C]

Synthesised following the same conditions used for compound 399.

### Compound 415: [4-Ethyl-6-((S)-3-fluoro-pyrrolidin-1-yl)-pyridin-3-yl]-methyl-(1-methyl-1H-imidazo[4,5-c]pyridin-6-yl)-amine

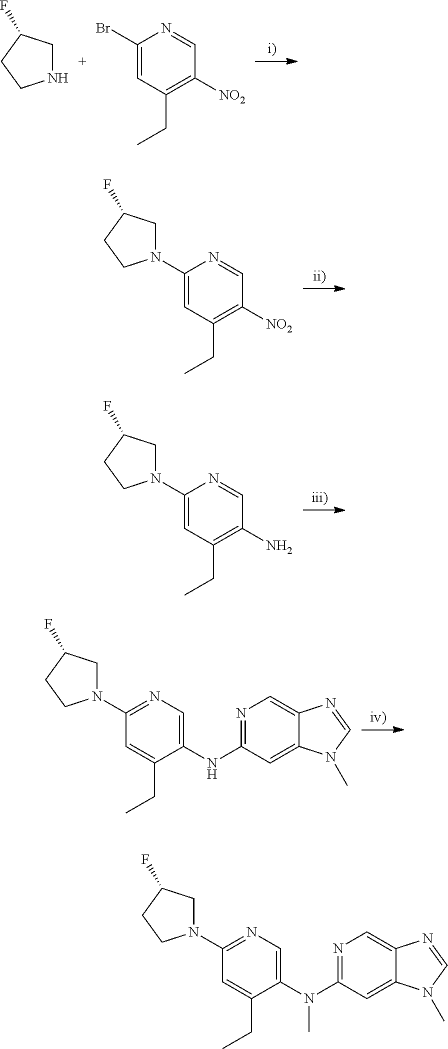 [SMILES: F[C@H]1CCNC1 ; CCc1cc(Br)ncc1[N+](=O)[O-] ; CCc1cc(N2CC[C@H](F)C2)ncc1[N+](=O)[O-] ; CCc1cc(N2CC[C@H](F)C2)ncc1N ; CCc1cc(N2CC[C@H](F)C2)ncc1Nc1cc2c(cn1)ncn2C ; CCc1cc(N2CC[C@H](F)C2)ncc1N(C)c1cc2c(cn1)ncn2C]

Synthesised following the same conditions used for compound 399.

### Compound 416: 4′-Ethyl-5′-[methyl-(1-methyl-1H-imidazo[4,5-c]pyridin-6-yl)-amino]-3,4,5,6-tetrahydro-2H-[1,2′]bipyridinyl-3-carbonitrile

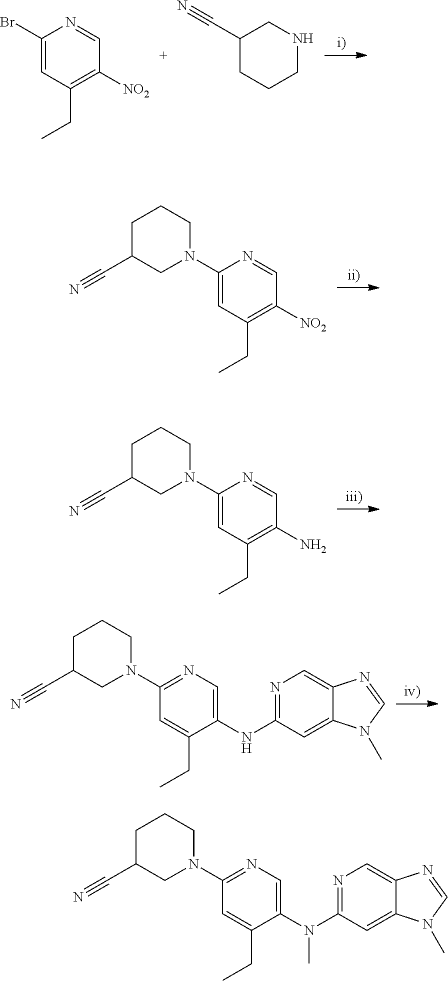

Synthesised following the same conditions used for compound 399.

### Compound 417: [4-Ethyl-6-((S)-3-methyl-pyrrolidin-1-yl)-pyridin-3-yl]-methyl-(1-methyl-1H-imidazo[4,5-c]pyridin-6-yl)-amine

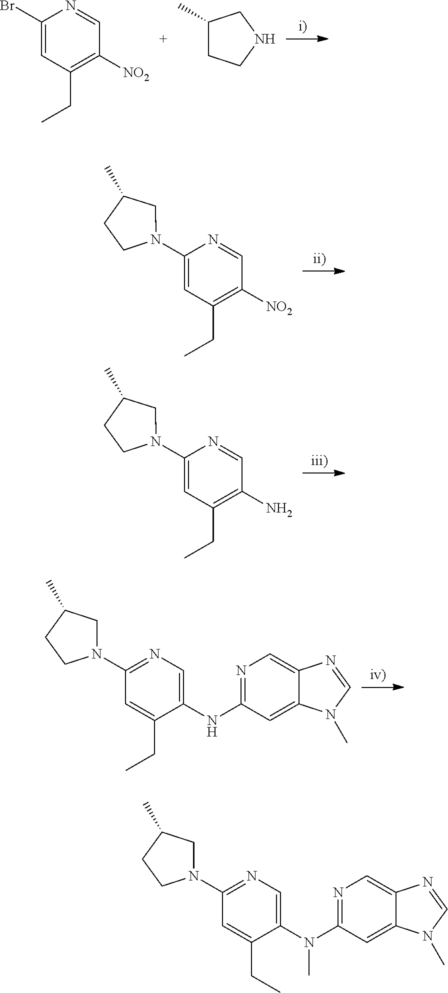

Synthesised following the same conditions used for compound 399.

### Compound 418: 1-{4-Ethyl-5-[methyl-(1-methyl-1H-imidazo[4,5-c]pyridin-6-yl)-amino]-pyridin-2-yl}-pyrrolidine-3-carbonitrile

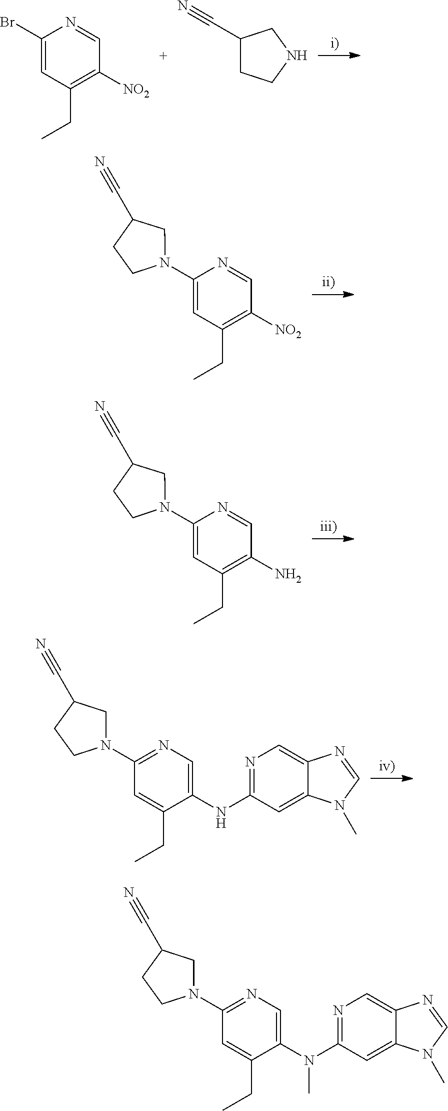

Synthesised following the same conditions used for compound 399.

### Compound 419: [2-Ethyl-4-(2-oxa-6-aza-spiro[3.3]hept-6-ylmethyl)-phenyl]-methyl-(1-methyl-1H-imidazo[4,5-c]pyridin-6-yl)-amine

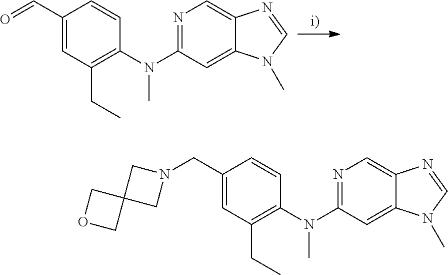 [SMILES: CCc1cc(C=O)ccc1N(C)c1cc2c(cn1)ncn2C ; CCc1cc(CN2CC3(COC3)C2)ccc1N(C)c1cc2c(cn1)ncn2C]

A solution of the aldehyde derivative (1 equiv, 110 mg), 2-oxa-6-aza-spiro[3.3]heptane oxalate salt (1.3 equiv, 72 mg) and AcOH (150 μL) in MeOH (5.5 mL) and DCM (4 mL) was stirred at room temperature for 3 h. Na(CN)BH₃ (0.5 equiv, 12 mg) was added and the mixture was stirred at room temperature. After 16 h, the reaction was concentrated and purification by preparative HPLC afforded the desired compound.

### Compound 420: [2-Ethyl-4-(3-methanesulfonyl-pyrrolidin-1-ylmethyl)-phenyl]-methyl-(1-methyl-1H-imidazo[4,5-c]pyridin-6-yl)-amine

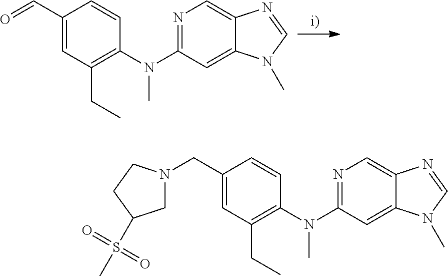 [SMILES: CCc1cc(C=O)ccc1N(C)c1cc2c(cn1)ncn2C ; CCc1cc(CN2CCC(S(C)(=O)=O)C2)ccc1N(C)c1cc2c(cn1)ncn2C]

A solution of the aldehyde derivative (1 equiv, 110 mg), 3-methanesulfonyl-pyrrolidine (1.3 equiv, 75 mg) and AcOH (150 μL) in MeOH (5.5 mL) and DCM (4 mL) was stirred at room temperature for 3 h. Na(CN)BH₃ (0.5 equiv, 12 mg) was added and the mixture was stirred at room temperature. After 16 h, the reaction was concentrated and purification by preparative HPLC afforded the desired compound.

### Compound 421: 4-{3-Ethyl-4-[methyl-(1-methyl-1H-imidazo[4,5-c]pyridin-6-yl)-amino]-benzyl}-piperazin-2-one

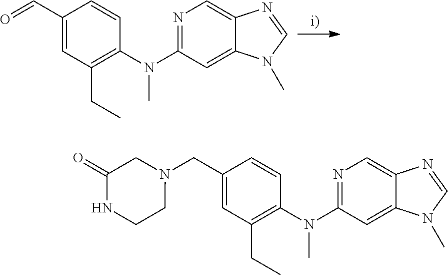

A solution of the aldehyde derivative (1 equiv, 100 mg), piperazin-2-one (1.3 equiv, 44 mg) and AcOH (150 μL) in MeOH (4 mL) and DCM (2.7 mL) was stirred at room temperature for 3 h. Na(CN)BH₃ (0.5 equiv, 11 mg) was added and the mixture was stirred at room temperature. After 16 h, the reaction was concentrated and purification by preparative HPLC afforded the desired compound.

### Compound 422: [4-(4,4-Difluoro-piperidin-1-ylmethyl)-2-ethyl-phenyl]-methyl-(1-methyl-1H-imidazo[4,5-c]pyridin-6-yl)-amine

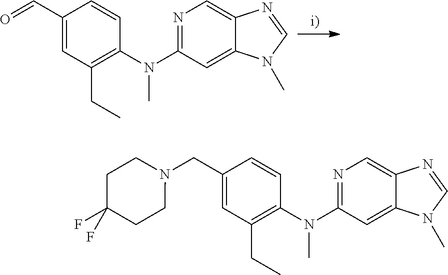

A solution of the aldehyde (1 equiv, 80 mg), 4,4′-difluoropiperidine (1.3 equiv, 55 mg) and AcOH (120 μL) in MeOH (3.75 mL) and DCM (2.5 mL) was stirred at room temperature for 3 h. Na(CN)BH₃ (0.5 equiv, 9 mg) was added and the mixture was stirred at room temperature. After 2 h, the reaction was concentrated and purification by preparative HPLC afforded the desired compound.

### Compound 423

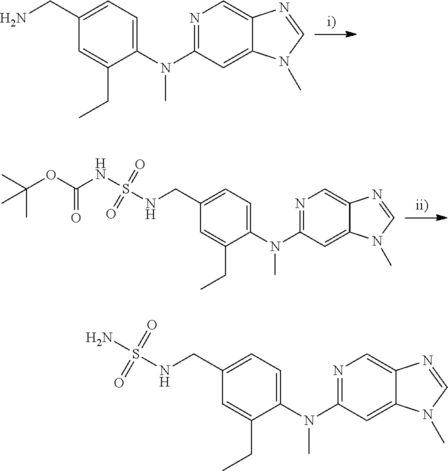 [SMILES: CCc1cc(CN)ccc1N(C)c1cc2c(cn1)ncn2C ; CCc1cc(CNS(=O)(=O)NC(=O)OC(C)(C)C)ccc1N(C)c1cc2c(cn1)ncn2C ; CCc1cc(CNS(N)(=O)=O)ccc1N(C)c1cc2c(cn1)ncn2C]

### Step (i)

A solution of tBuOH (33 μL) in DCM (0.2 mL) was added dropwised to a solution of chlorosulfonyl isocyanate (1 equiv, 30 μL) in DCM (0.2 mL) at 0° C. and the mixture was stirred at room temperature for 30 min. This mixture was added to a mixture of benzylamine (1 equiv, 100 mg), NEt₃ (1.1 equiv, 52 μL) in DCM (0.7 mL) at 0° C. and the resulting mixture was stirred at room temperature for 6 h. The mixture was concentrated, diluted with water and extracted with EtOAc. The organic extract was dried and concentrated to give the desired compound.

### Step (ii)

HCl (4M in dioxane, 0.4 mL) was added to a solution of the Boc-protected sulfamoyl (1 equiv, 161 mg) in 1,4-dioxane (0.5 mL) and the reaction was stirred at room temperature for 1.5 h. The mixture was then concentrated and purified by preparative HPLC to afford the desired compound.

### Compound 424: 3,3-Difluoro-azetidine-1-sulfonic acid 3-ethyl-4-[methyl-(1-methyl-1H-imidazo[4,5-c]pyridin-6-yl)-amino]-benzylamide

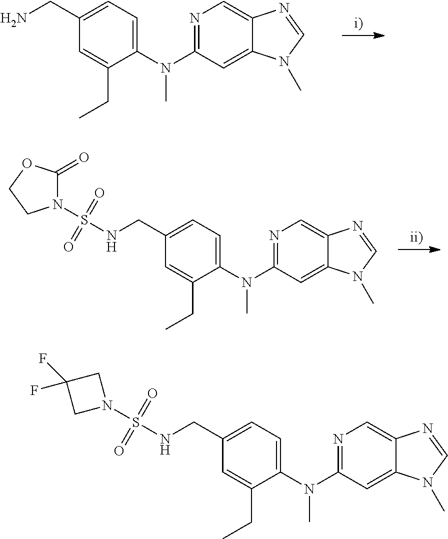

### Step (i)

A solution of 2-bromoethanol (1 equiv, 24 μL) in DCM (0.19 mL) was added dropwised to a solution of chlorosulfonyl isocyanate (1 equiv, 30 μL) in DCM (0.68 mL) at 0° C. and the mixture was stirred at 0° C. for 30 min. This mixture was added to a mixture of benzylamine (1 equiv, 100 mg), NEt₃ (2 equiv, 95 μL) in DCM (0.38 mL) at 0° C. and the resulting mixture was stirred at room temperature for 30 min. The mixture was washed with 2M HCl (3×) and water and the solvent was removed under vacuum. The aqueous layer was neutralized and concentrated. Both organic and aqueous fraction were combined and purified by silica chromatography (7M NH₃ in MeOH/ErOAc) to afford the desired compound.

### Step (ii)

Difluoroazetidine hydrochloride (1.1 equiv, 26 mg) and NEt₃ (2.2 nequiv, 56 μL) were added to a solution of the sulfamoyl (1 equiv, 80 mg) in acetonitrile (2 mL) and the mixture was stirred at 85° C. for 18 h. The resulting mixture was then concentrated and purified by preparative HPLC to afford the desired compound.

### Compound 425: 3,3-Difluoro-pyrrolidine-1-sulfonic acid 3-ethyl-4-[methyl-(1-methyl-1H-imidazo[4,5-c]pyridin-6-yl)-amino]-benzylamide

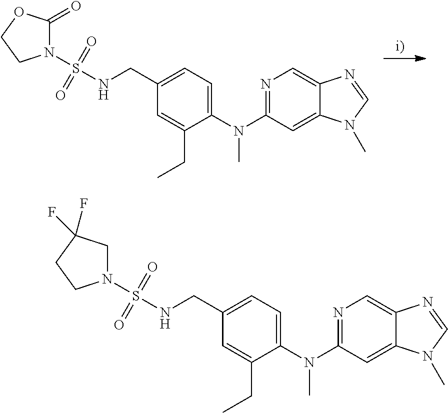 [SMILES: CCc1cc(CNS(=O)(=O)N2CCOC2=O)ccc1N(C)c1cc2c(cn1)ncn2C ; CCc1cc(CNS(=O)(=O)N2CCC(F)(F)C2)ccc1N(C)c1cc2c(cn1)ncn2C]

3,3-Difluoro-pyrrolidine hydrochloride (1.1 equiv, 29 mg) and NEt₃ (2.2 equiv, 56 μL) were added to a solution of the sulfamoyl (1 equiv, 80 mg) in acetonitrile (2 mL) and the mixture was stirred at 85° C. for 18 h. The resulting mixture was then concentrated and purified by preparative HPLC to afford the desired compound.

### Compound 426: Morpholine-4-sulfonic acid 3-ethyl-4-[methyl-(1-methyl-1H-imidazo[4,5-c]pyridin-6-yl)-amino]-benzylamide

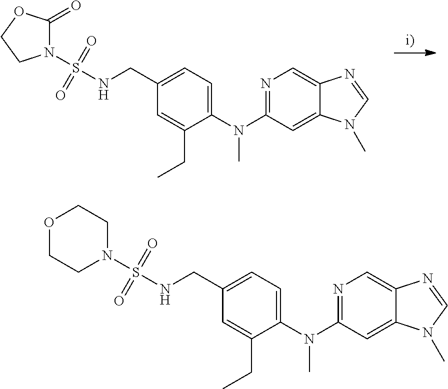 [SMILES: CCc1cc(CNS(=O)(=O)N2CCOC2=O)ccc1N(C)c1cc2c(cn1)ncn2C ; CCc1cc(CNS(=O)(=O)N2CCOCC2)ccc1N(C)c1cc2c(cn1)ncn2C]

Morpholine (1.1 equiv, 18 μL) and NEt₃ (2.2 nequiv, 56 μL) were added to a solution of the sulfamoyl (1 equiv, 80 mg) in acetonitrile (2 mL) and the mixture was stirred at 85° C. for 18 h. The resulting mixture was then concentrated and purified by preparative HPLC to afford the desired compound.

### Compound 427: (2-Difluoromethoxy-4-methyl-phenyl)-methyl-(1-methyl-1H-imidazo[4,5-c]pyridin-6-yl)-amine

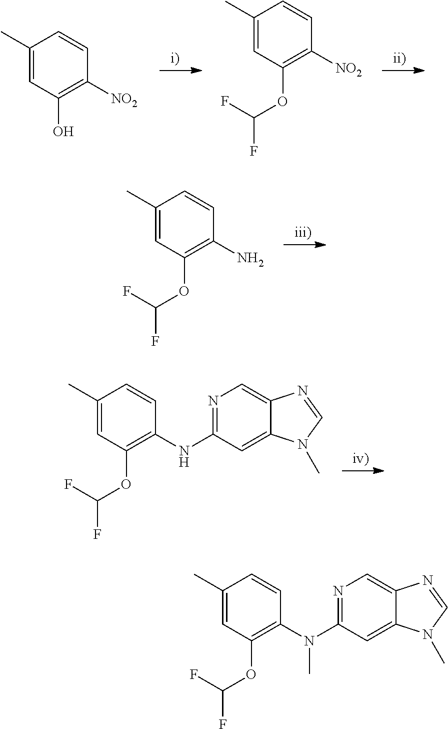 [SMILES: Cc1ccc([N+](=O)[O-])c(O)c1 ; Cc1ccc([N+](=O)[O-])c(OC(F)F)c1 ; Cc1ccc(N)c(OC(F)F)c1 ; Cc1ccc(Nc2cc3c(cn2)ncn3C)c(OC(F)F)c1 ; Cc1ccc(N(C)c2cc3c(cn2)ncn3C)c(OC(F)F)c1]

### Step (i): 2-Difluoromethoxy-4-methyl-1-nitro-benzene

Diethyl(bromodifluoromethyl)phosphonate (2 equiv, 2.5 mL) was added to a cooled (−78° C.) solution of 5-methyl-2-nitrophenol (1 equiv, 1.1 g) and KOH (20 equiv, 7.8 g) in MeCN/water (72 mL; 1:1) and the reaction mixture was allowed to warm to room temperature. After 30 min, the mixture was diluted with Et₂O, the organic phase was separated and the water phase was washed with a further amount of Et₂O. The combined organics were dried and concentrated. The residue was purified by silica chromatography (EtOAc/cyclohexane; 2:95 to 50:50) to give the desired product.

### Step (ii): 2-Difluoromethoxy-4-methyl-phenylamine

A mixture of the nitro-aryl (1 equiv, 50 mg) and palladium on carbon (10% wt., 7.5 mg) in EtOH (0.6 mL) was stirred under a hydrogen atmosphere at room temperature for 2 h. The reaction mixture was filtered through Celite and the filtrate was concentrated to afford the desired product.

### Step (iii): (2-Difluoromethoxy-4-methyl-phenyl)-(1-methyl-1H-imidazo[4,5-c]pyridin-6-yl)-amine

A degassed mixture of the amine (1 equiv, 28 mg), 6-chloro-1-methyl-1H-imidazo[4,5-c]pyridine (1 equiv, 27 mg), Cs₂CO₃ (2.5 equiv, 132 mg), Xphos (0.3 equiv, 23 mg) and Pd₂(dba)₃ (0.1 equiv, 15 mg) in dry DMF (1 mL) was heated at 110° C. for 18 h. Water was added and the reaction mixture was extracted with EtOAc (3×). The combined organics were dried, concentrated and the residue was purified by silica chromatography (MeOH/EtOAc; 0:100 to 10:90) to give the desired compound.

### Step iv: (2-Difluoromethoxy-4-methyl-phenyl)-methyl-(1-methyl-1H-imidazo[4,5-c]pyridin-6-yl)-amine

Synthesised following the same conditions used for compound 385 (step (v)).

### Compound 428: 3-Difluoromethoxy-4-[methyl-(1-methyl-1H-imidazo[4,5-c]pyridin-6-yl)-amino]-benzonitrile

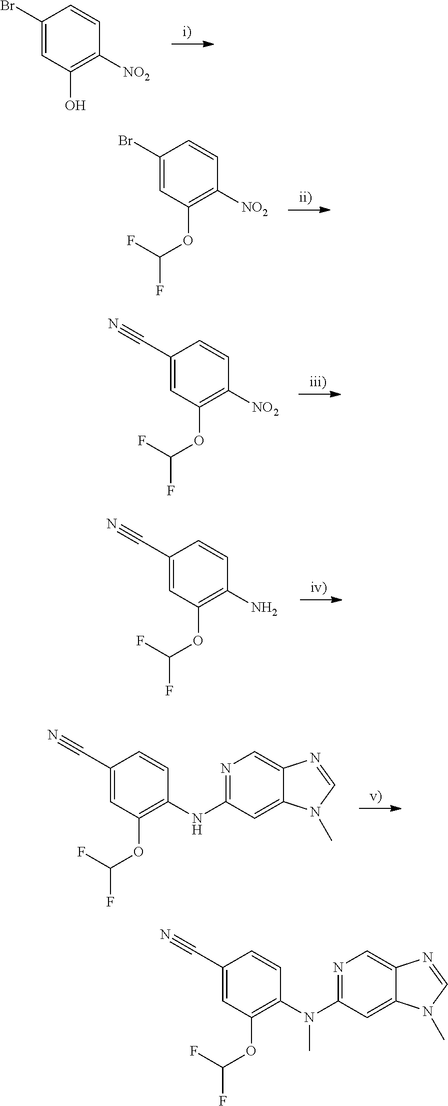 [SMILES: O=[N+]([O-])c1ccc(Br)cc1O ; O=[N+]([O-])c1ccc(Br)cc1OC(F)F ; N#Cc1ccc([N+](=O)[O-])c(OC(F)F)c1 ; N#Cc1ccc(N)c(OC(F)F)c1 ; Cn1cnc2cnc(Nc3ccc(C#N)cc3OC(F)F)cc21 ; CN(c1cc2c(cn1)ncn2C)c1ccc(C#N)cc1OC(F)F]

### Step (i): 4-Bromo-2-difluoromethoxy-1-nitro-benzene

Synthesised following the same conditions used for compound 427 (step (i)).

### Step (ii): 3-Difluoromethoxy-4-nitro-benzonitrile

A solution of the bromoaryl (1 equiv, 1.04 g) and Zn(CN)₂ (0.75 equiv, 0.34 g) in dry DMF (8.3 mL) and DMA (2.9 mL) was stirred at room temperature under argon for 10 min and then heated to 90° C. for 24 h. Water was added and the mixture extracted with EtOAc (3×), dried and concentrated. Purification by silica chromatography (EtOAc/cyclohexane; 3:97 to 30:70) gave the desired compound.

### Step (iii), (iv) and (v): 3-Difluoromethoxy-4-[methyl-(1-methyl-1H-imidazo[4,5-c]pyridin-6-yl)-amino]-benzonitrile

Synthesised following the same conditions used for compound 427 (step (ii)), compound 328 (step (ii)) and compound 385 (step (v)).

### Compound 429: N-{3-Difluoromethoxy-4-[methyl-(1-methyl-1H-imidazo[4,5-c]pyridin-6-yl)-amino]-benzyl}-methanesulfonamide

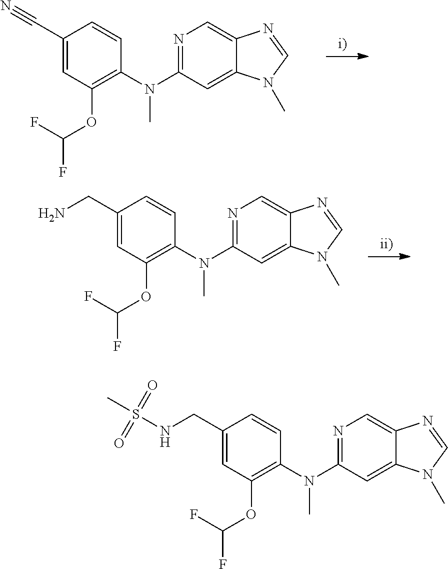

Synthesised following the same conditions used for compound 404.

### Compound 430: N-{3-Difluoromethoxy-4-[methyl-(1-methyl-1H-imidazo[4,5-c]pyridin-6-yl)-amino]-benzyl}-N-methyl-methanesulfonamide

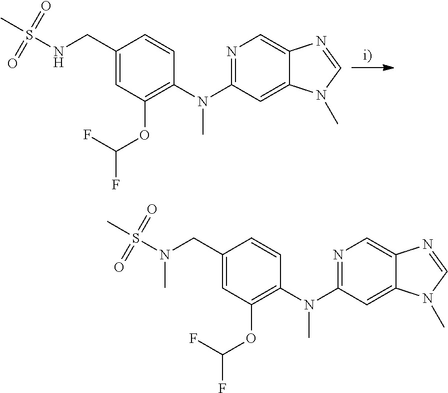

Sodium hydride (60%, 1.5 equiv, 3 mg) was added to a solution of the sulfonamide (1 equiv, 21 mg) in DMF (1 mL) at 0° C. After 20 min, MeI (1.15 equiv, 4 μL) was added and the reaction mixture was stirred at room temperature for 16 h. Water was added and the mixture was extracted with EtOAc (3×), dried and concentrated. Purification by silica chromatography (MeOH/DCM; 0.5:99.5 to 5:95) afforded the desired compound.

### Compound 431: (1-Difluoromethyl-1H-imidazo[4,5-c]pyridin-6-yl)-(2-ethyl-phenyl)-methyl-amine

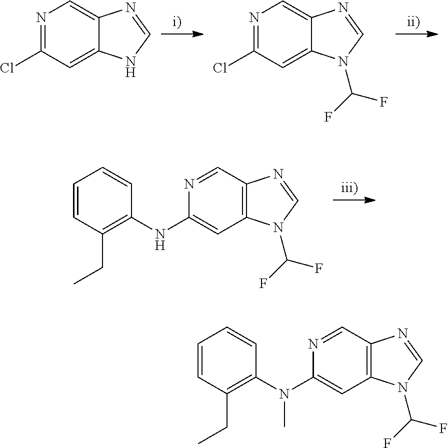 [SMILES: Clc1cc2[nH]cnc2cn1 ; FC(F)n1cnc2cnc(Cl)cc21 ; CCc1ccccc1Nc1cc2c(cn1)ncn2C(F)F ; CCc1ccccc1N(C)c1cc2c(cn1)ncn2C(F)F]

### Step (i): 6-Chloro-1-difluoromethyl-1H-imidazo[4,5-c]pyridine

Diethyl(bromodifluoromethyl)phosphonate (2 equiv, 0.46 mL) was added to a cooled (0° C.) solution of 6-chloro-1H-imidazo[4,5-c]pyridine (1 equiv, 200 mg), KOH (20 equiv, 1.5 g) in MeCN (10 mL) and H₂O (10 mL) and the reaction mixture was allowed to warm to room temperature. After 30 min, the resulting mixture was diluted with DCM, the layer were separated and the aqueous further extracted with DCM (3×), dried and concentrated. Purification by silica chromatography (EtOAc/petrol ether; 0:100 to 30:70) afforded a mixture of two isomers.

### Step (ii) and (iii): (1-Difluoromethyl-1H-imidazo[4,5-c]pyridin-6-yl)-(2-ethyl-phenyl)-amine

A mixture of 2-ethylaniline (1 equiv, 50 mg), imidazolopyridine (1.15 equiv, 100 mg), BINAP (0.3 equiv, 80 mg) and Cs₂CO₃ (3 equiv, 420 mg) in dry 1,4-dioxane (1.5 mL) was degassed for 10 min followed by the addition of Pd₂ dba₃ (0.2 equiv, 80 mg). The mixture was degassed and then stirred at 100° C. for 16 h. The mixture was diluted with DCM, filtered through Celite and the filtrate concentrated. The residue was rediluted in DCM, washed with aq. sat. NaHCO₃ solution, dried, filtered and concentrated. The crude was dissolved in dry THF (1 mL) followed by the addition of NaH (3 equiv, 50 mg) and iodomethane (3 equiv, 80 μL) and the mixture was stirred at room temperature for 3 h. The resulting mixture was diluted with DCM, washed with water, with aq. sat. NaHCO₃ solution, dried and concentrated. Purification preparative HPLC afforded the desired compound.

### Compound 432: (6-Cyclohexyl-4-ethyl-pyridin-3-yl)-methyl-(1-methyl-1H-imidazo[4,5-c]pyridin-6-yl)-amine

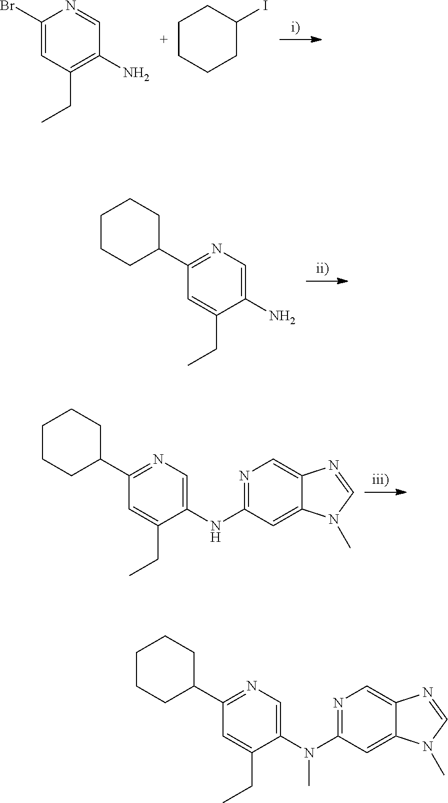

### Step (i): 6-Cyclohexyl-4-ethyl-pyridin-3-ylamine

Iodo-cyclohexane (1.9 equiv, 150 mg) was dissolved in THF (4 mL) and heated at 65° C. Under atmosphere of nitrogen, Rieke zinc (5 g/100 mL, 2.0 equiv, 0.84 mL) was added dropwise and stirred for 3 h at 65° C. under nitrogen. In another flask 6-bromo-4-ethyl-pyridin-3-ylamine (1 equiv, 72 mg), copper(I) iodide (0.1 equiv, 6.8 mg) and PdCl₂dppf (0.05 equiv, 14.6 mg) were dissolved in THF (4 mL) and heated at 85° C. under atmosphere of nitrogen. The reaction mixture from the first flask was added dropwised via a cannule to the second flask. The resulting reaction mixture was stirred at 85° C. for 2 h. The reaction was then quenched with water and extracted with EtOAc (3×). The combined organics was dried and concentrated under reduced pressure. The residue was purified by column chromatography (MeOH/DCM; 0:100 to 10:90) to give the expected aniline.

### Step (ii) and (iii): (6-Cyclohexyl-4-ethyl-pyridin-3-yl)-methyl-(1-methyl-1H-imidazo[4,5-c]pyridin-6-yl)-amine

Synthesised following the same conditions used for compound 328 (step (ii)) and compound 385 (step (v)).

### Compound 433: (6-Cyclopentyl-4-ethyl-pyridin-3-yl)-methyl-(1-methyl-1H-imidazo[4,5-c]pyridin-6-yl)-amine

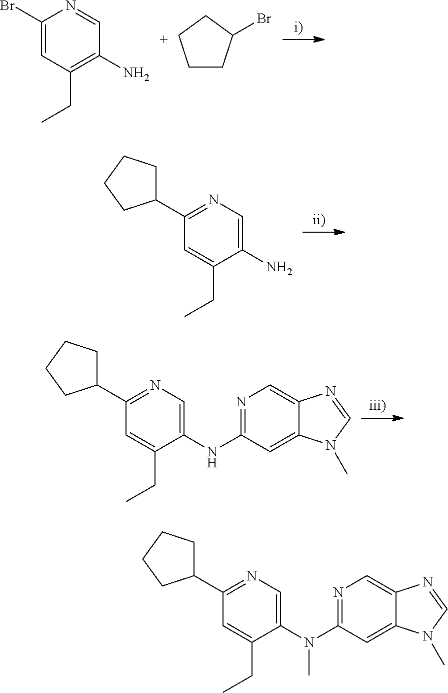

### Step (i): 6-Cyclopentyl-4-ethyl-pyridin-3-ylamine

Bromo-cyclopentane (3 equiv, 445 mg) was dissolved in THF (4 mL) and heated at 65° C. Under atmosphere of nitrogen, Rieke zinc (5 g/100 mL, 2.9 equiv, 3.7 mL) was added dropwise and stirred for 2 h at 65° C. under nitrogen. In another flask 6-bromo-4-ethyl-pyridin-3-ylamine (1 equiv, 200 mg), copper(I) iodide (0.1 equiv, 18.8 mg) and PdCl₂dppf (0.05 equiv, 40.4 mg) were dissolved in THF (4 mL) and heated at 85° C. under atmosphere of nitrogen. The reaction mixture from the first flask was added dropwised via a cannule to the second flask. The resulting reaction mixture was stirred at 85° C. for 18 h. The reaction was then quenched with water and extracted with EtOAc (3×). The combined organics was dried and concentrated under reduced pressure. The residue was purified by column chromatography (MeOH/DCM; 0:100 to 10:90) to give the expected aniline.

### Step (ii) and (iii): ((6-Cyclopentyl-4-ethyl-pyridin-3-yl)-methyl-(1-methyl-1H-imidazo[4,5-c]pyridin-6-yl)-amine

Synthesised following the same conditions used for compound 328 (step (ii)) and compound 385 (step (v)).

### Compound 434: (6-Cyclobutyl-4-ethyl-pyridin-3-yl)-methyl-(1-methyl-1H-imidazo[4,5-c]pyridin-6-yl)-amine

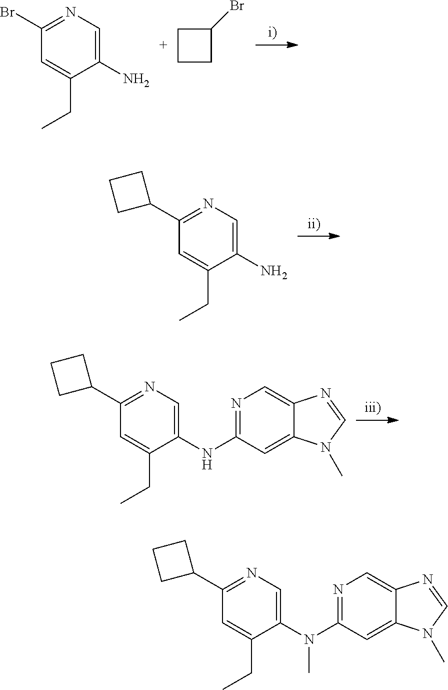

Synthesised following the same conditions used for compound 328 (step (ii)) and compound 385 (step (v)).

### Compound 435: 1-(Cyano-{3-ethyl-4-[methyl-(1-methyl-1H-imidazo[4,5-c]pyridin-6-yl)-amino]-phenyl}-methyl)-piperidine-4-carbonitrile

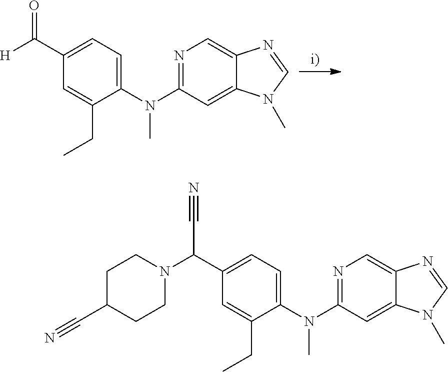 [SMILES: CCc1cc(C=O)ccc1N(C)c1cc2c(cn1)ncn2C ; CCc1cc(C(C#N)N2CCC(C#N)CC2)ccc1N(C)c1cc2c(cn1)ncn2C]

A mixture of the aldehyde derivative (1 equiv, 50 mg), 4-cyanopiperidine (1 equiv, 20 mg), zinc cyanide (1 equiv, 20 mg) and DIPEA (1 equiv, 30 μL) in EtOH (0.6 mL) and AcOH (0.3 mL) was irradiated in microwave Biotage Initiator at 100° C. for 30 min. Purification by silica chromatography (EtOAc) afforded the desired compound.

### Compound 436: [1-(Cyano-{3-ethyl-4-[methyl-(1-methyl-1H-imidazo[4,5-c]pyridin-6-yl)-amino]-phenyl}-methyl)-azetidin-3-yl]-carbamic acid tert-butyl ester

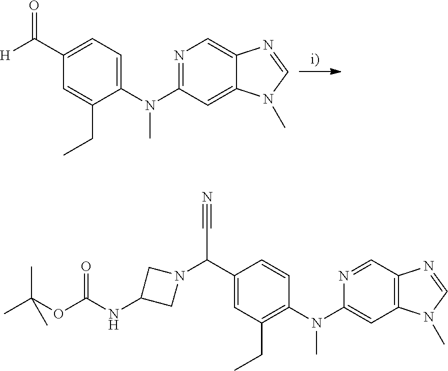 [SMILES: CCc1cc(C=O)ccc1N(C)c1cc2c(cn1)ncn2C ; CCc1cc(C(C#N)N2CC(NC(=O)OC(C)(C)C)C2)ccc1N(C)c1cc2c(cn1)ncn2C]

A mixture of the aldehyde derivative (1 equiv, 50 mg), 3-boc-aminoazetidine hydrochloride (1 equiv, 35 mg), zinc cyanide (1 equiv, 20 mg) and DIPEA (1 equiv, 30 μL) in EtOH (0.3 mL) and AcOH (0.3 mL) was irradiated in microwave Biotage Initiator at 100° C. for 20 min. Purification by silica chromatography (EtOAc) afforded the desired compound.

### Compound 437: {3-Ethyl-4-[methyl-(1-methyl-1H-imidazo[4,5-c]pyridin-6-yl)-amino]-phenyl}-(4-hydroxymethyl-piperidin-1-yl)-acetonitrile

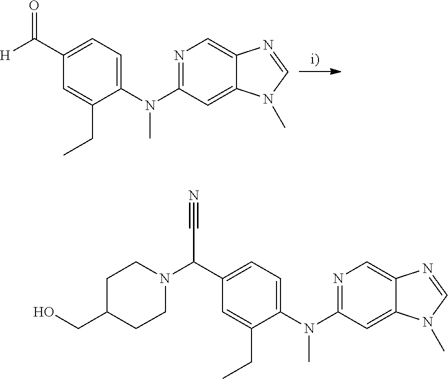 [SMILES: CCc1cc(C=O)ccc1N(C)c1cc2c(cn1)ncn2C ; CCc1cc(C(C#N)N2CCC(CO)CC2)ccc1N(C)c1cc2c(cn1)ncn2C]

A mixture of the aldehyde derivative (1 equiv, 50 mg), 4-piperidinmethanol (1 equiv, 20 mg), zinc cyanide (1 equiv, 20 mg) and DIPEA (1 equiv, 30 μL) in EtOH (0.3 mL) and AcOH (0.3 mL) was irradiated in microwave Biotage Initiator at 100° C. for 30 min. Purification by silica chromatography (EtOAc) afforded the desired compound.

### Compound 438: {3-Ethyl-4-[methyl-(1-methyl-1H-imidazo[4,5-c]pyridin-6-yl)-amino]-phenyl}-((R)-3-fluoro-pyrrolidin-1-yl)-acetonitrile

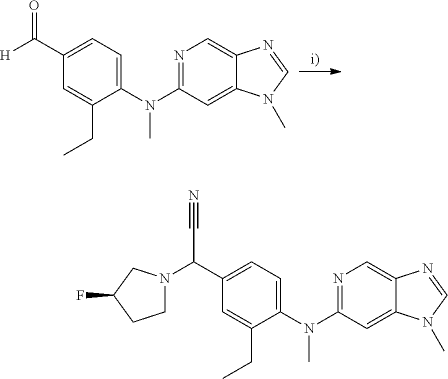 [SMILES: CCc1cc(C=O)ccc1N(C)c1cc2c(cn1)ncn2C ; CCc1cc(C(C#N)N2CC[C@@H](F)C2)ccc1N(C)c1cc2c(cn1)ncn2C]

A mixture of the aldehyde derivative (1 equiv, 50 mg), (S)-3-fluoropyrrolidine hydrochloride (1 equiv, 21 mg), zinc cyanide (1 equiv, 20 mg) and DIPEA (1 equiv, 30 μL) in EtOH (0.3 mL) and AcOH (0.3 mL) was irradiated in microwave Biotage Initiator at 100° C. for 30 min. Purification by silica chromatography (EtOAc) afforded the desired compound.

### Compound 439: {3-Ethyl-4-[methyl-(1-methyl-1H-imidazo[4,5-c]pyridin-6-yl)-amino]-phenyl}-((R)-2-methoxymethyl-pyrrolidin-1-yl)-acetonitrile

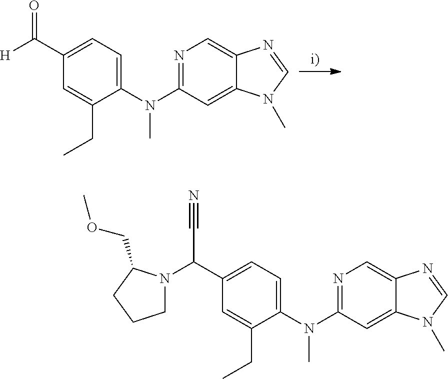

A mixture of the aldehyde derivative (1 equiv, 50 mg), (R)-2-(methoxymethyl) pyrrolidine (1 equiv, 21 μL), zinc cyanide (1 equiv, 20 mg) and DIPEA (1 equiv, 30 μL) in EtOH (0.3 mL) and AcOH (0.3 mL) was irradiated in microwave Biotage Initiator at 100° C. for 30 min. Purification by silica chromatography (EtOAc) afforded the desired compound.

### Compound 440: (3,3-Difluoro-azetidin-1-yl)-{3-ethyl-4-[methyl-(1-methyl-1H-imidazo[4,5-c]pyridin-6-yl)-amino]-phenyl}-acetonitrile

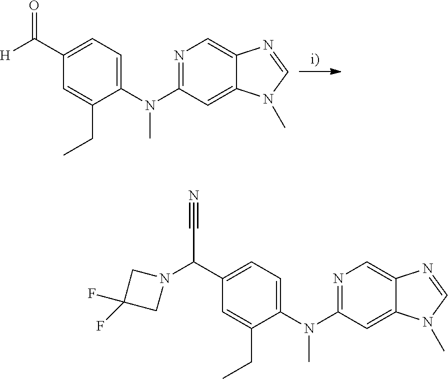 [SMILES: CCc1cc(C=O)ccc1N(C)c1cc2c(cn1)ncn2C ; CCc1cc(C(C#N)N2CC(F)(F)C2)ccc1N(C)c1cc2c(cn1)ncn2C]

A mixture of the aldehyde derivative (1 equiv, 50 mg), 3,3-difluoroazetidine hydrochloride (1 equiv, 22 mg), zinc cyanide (1 equiv, 20 mg) and DIPEA (1 equiv, 30 μL) in EtOH (0.3 mL) and AcOH (0.3 mL) was irradiated in microwave Biotage Initiator at 100° C. for 30 min. Purification by silica chromatography (EtOAc) afforded the desired compound.

### Compound 441: (3,3-Difluoro-pyrrolidin-1-yl)-{3-ethyl-4-[methyl-(1-methyl-1H-imidazol[4,5-d]pyridin-6-yl)-amino]-phenyl}-acetonitrile

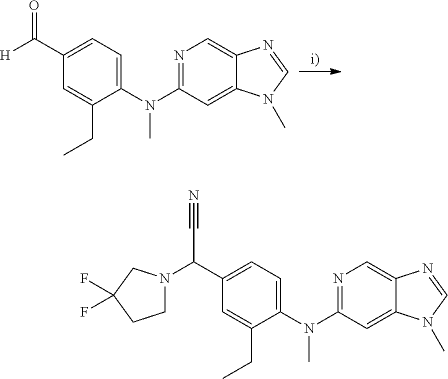 [SMILES: CCc1cc(C=O)ccc1N(C)c1cc2c(cn1)ncn2C ; CCc1cc(C(C#N)N2CCC(F)(F)C2)ccc1N(C)c1cc2c(cn1)ncn2C]

A mixture of the aldehyde derivative (1 equiv, 50 mg), 3,3-difluoropyrrolidine hydrochloride (1 equiv, 24 mg), zinc cyanide (1 equiv, 20 mg) and DIPEA (1 equiv, 30 μL) in EtOH (0.3 mL) and AcOH (0.3 mL) was irradiated in microwave Biotage Initiator at 100° C. for 30 min. Purification by silica chromatography (EtOAc) afforded the desired compound.

### Compound 442: [6-(3-Amino-azetidin-1-yl)-4-ethyl-pyridin-3-yl]-methyl-(1-methyl-1H-imidazo[4,5-d]pyridin-6-yl)-amine

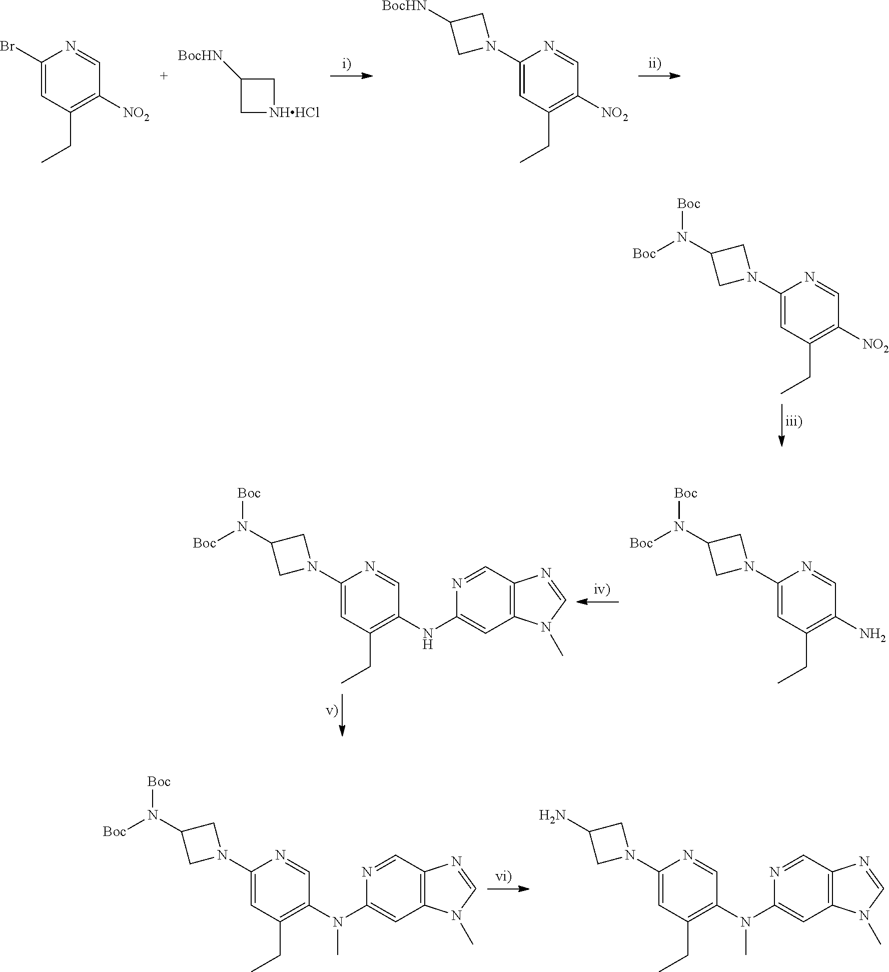

### Step i): [(4-Ethyl-5-nitro-pyridin-2-yl)-azetidin-3-yl]-carbamic acid tert-butyl ester

A mixture of 2-bromo-4-ethyl-5-nitro-pyridine (1 equiv, 101 mg), azetidin-3-yl-carbamic acid tert-butyl ester hydrochloride (1.1 equiv, 100 mg) and K₂CO₃ (2.5 equiv, 151 mg) in DMF (0.73 mL) was stirred at 70° C. for 1 h. Water was added, the mixture was extracted with DCM (3×), dried and concentrated.

### Step ii)

A mixture of the Boc-protected amine (1 equiv, 132 mg), Boc₂O (3.2 equiv, 285 mg) and DMAP (0.4 equiv, 20 mg) in MeCN (1.4 mL) was heated at reflux temperature for 4 h. After removal of the solvent, water was added and the mixture extracted with DCM (3×), dried and concentrated. Purification by silica chromatography (EtOAc/hexane; 0:100 to 30:70) afforded the compound.

### Step iii), iv) and v)

Synthesised following the same conditions used for compound 399.

### Step vi): [6-(3-Amino-azetidin-1-yl)-4-ethyl-pyridin-3-yl]-methyl-(1-methyl-1H-imidazo[4,5-c]pyridin-6-yl)-amine

TFA (0.55 mL) was added to a solution of the bis-Boc-protected amine (1 equiv, 55 mg) in DCM (1.4 mL) and the mixture was stirred at room temperature for 18 h. Purification by SCX column afforded the desired compound.

### Compound 443: N-(1-{4-Ethyl-5-[methyl-(1-methyl-1H-imidazo[4,5-c]pyridin-6-yl)-amino]-pyridin-2-yl}-azetidin-3-yl)-methAnesulfonamide

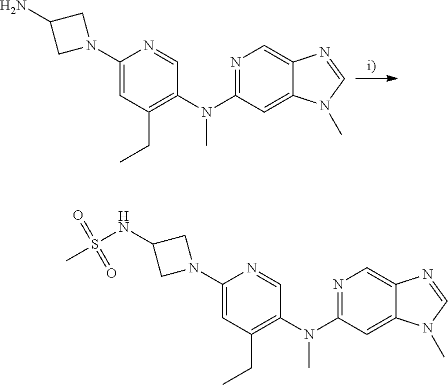 [SMILES: CCc1cc(N2CC(N)C2)ncc1N(C)c1cc2c(cn1)ncn2C ; CCc1cc(N2CC(NS(C)(=O)=O)C2)ncc1N(C)c1cc2c(cn1)ncn2C]

Methanesulfonyl chloride (1.1 equiv, 9 μL) was added portiowised to a solution of the amine (1 equiv, 28 mg) and NEt₃ (3 equiv, 42 μL) at 0° C. After stirring at 0° C. for 3 h, water was added, the resulting mixture extracted with DCM (3×), dried and concentrated. Purification by silica chromatography (MeOH/DCM; 0:100 to 3:97) afforded the desired compound.

### Compound 444: {4-Ethyl-5-[methyl-(1-methyl-1H-imidazo[4,5-c]pyridin-6-yl)-amino]-pyridin-2-yl}-morpholin-4-yl-acetonitrile

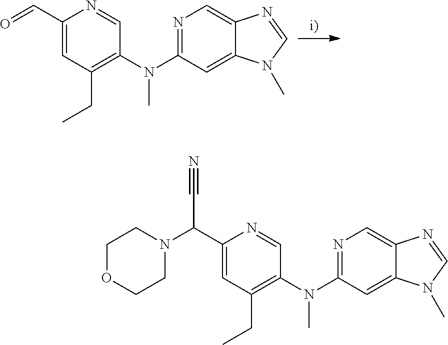 [SMILES: CCc1cc(C=O)ncc1N(C)c1cc2c(cn1)ncn2C ; CCc1cc(C(C#N)N2CCOCC2)ncc1N(C)c1cc2c(cn1)ncn2C]

Morpholine (3 equiv, 44 μL) was added to a mixture of the aldehyde (1 equiv, 49 mg) and LiClO₄ (2 equiv, 35 mg) in MeCN (0.9 mL). After stirring 5 min at room temperature, TMSCN (1.2 equiv, 25 μL) was added and the resulting mixture was stirred at room temperature for 18 h. After concentration, the residue was purified by silica chromatography (MeOH/DCM; 0:100 to 8:92) to afford the desired compound.

### Compound 445: 1-(Cyano-{3-ethyl-4-[methyl-(1-methyl-1H-imidazo[4,5-c]pyridin-6-yl)-amino]-phenyl}-methyl)-pyrrolidine-3-carbonitrile

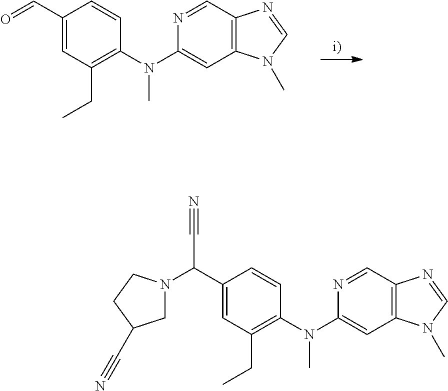 [SMILES: CCc1cc(C=O)ccc1N(C)c1cc2c(cn1)ncn2C ; CCc1cc(C(C#N)N2CCC(C#N)C2)ccc1N(C)c1cc2c(cn1)ncn2C]

Pyrrolidine-3-carbonitirile hydrochloride (3 equiv, 68 mg) was added to a mixture of the aldehyde derivative (1 equiv, 50 mg) and LiClO₄ (2 equiv, 36 mg) in MeCN (0.9 mL). After stilling 5 min at room temperature, TMSCN (1.2 equiv, 26 μL) was added and the resulting mixture was stirred at room temperature for 18 h. After concentration, the residue was purified by silica chromatography (EtOAc/cyclohexane; 0:100 to 100:0) to afford the desired compound.

### Compound 384: {4-Ethyl-5-(methyl-(1-methyl-1H-imidazo[4,5-c]pyridin-6-yl)-amino)-3′,4′,5′,6′-tetrahydro-2′H-[2,4′]bipyridinyl-1′-yl}-acetonitrile

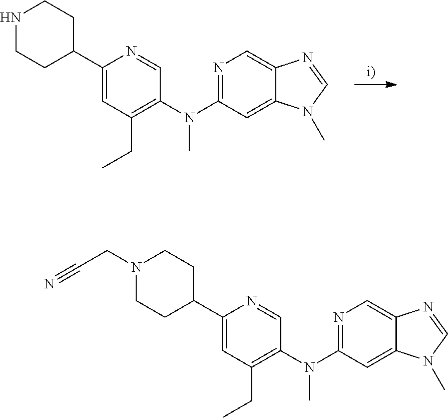 [SMILES: CCc1cc(C2CCNCC2)ncc1N(C)c1cc2c(cn1)ncn2C ; CCc1cc(C2CCN(CC#N)CC2)ncc1N(C)c1cc2c(cn1)ncn2C]

Bromoacetonitrile (1 equiv, 4 μL) was added to a mixture of the piperidine derivative (1 equiv, 20 mg) and K₂CO₃ (2 equiv, 16 mg) in MeCN (0.5 mL) and the reaction was stirred at room temperature for 16 h. The mixture was diluted with EtOAc, washed with water (3×), dried and concentrated. Purification by silica chromatography (NH₃/MeOH/EtOAc; 0.1:1:5) afforded the desired compound.

### Compound 446: (3,3-Dimethyl-azetidin-1-yl)-{3-ethyl-4-[methyl-(1-methyl-1H-imidazo[4,5-c]pyridin-6-yl)-amino]-phenyl}-acetonitrile

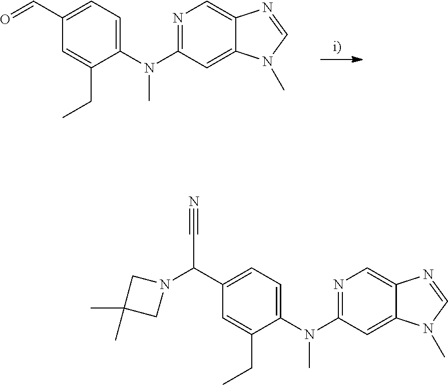 [SMILES: CCc1cc(C=O)ccc1N(C)c1cc2c(cn1)ncn2C ; CCc1cc(C(C#N)N2CC(C)(C)C2)ccc1N(C)c1cc2c(cn1)ncn2C]

A mixture of the aldehyde derivative (1 equiv, 50 mg), 3-dimethyl-azetidine hydrochloride (3 equiv, 62 mg), zinc cyanide (1 equiv, 20 mg) and DIPEA (3 equiv, 89 μL) in EtOH (2.4 mL) and AcOH (3.1 mL) was irradiated in microwave Biotage Initiator at 100° C. for 20 min. Water was added, the mixture extracted with DCM (3×), dried and concentrated. Purification by silica chromatography (EtOAc) afforded the desired compound.

### Compound 447: {3-Ethyl-4-[methyl-(1-methyl-1H-imidazo[4,5-c]pyridin-6-yl)-amino]-phenyl}-(4-hydroxy-piperidin-1-yl)-acetonitrile

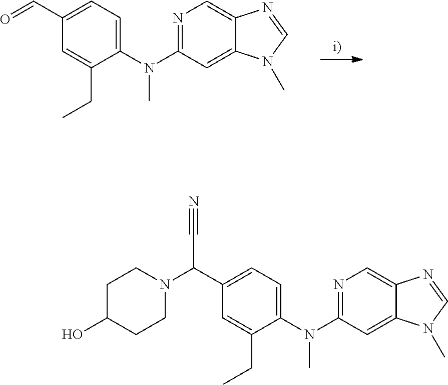 [SMILES: CCc1cc(C=O)ccc1N(C)c1cc2c(cn1)ncn2C ; CCc1cc(C(C#N)N2CCC(O)CC2)ccc1N(C)c1cc2c(cn1)ncn2C]

Piperidin-4-ol (3 equiv, 31 mg) was added to a mixture of the aldehyde deriavtive (1 equiv, 30 mg) and LiClO₄ (2 equiv, 22 mg) in MeCN (0.5 mL). After stirring 5 min at room temperature, TMSCN (1.2 equiv, 17 μL) was added and the resulting mixture was stirred at room temperature for 18 h. After concentration, the residue was purified by silica chromatography (MeOH/DCM; 0:100 to 20:80) to afford the desired compound.

### Compound 448: {3-Ethyl-4-[methyl-(1-methyl-1H-imidazo[4,5-c]pyridin-6-yl)-amino]-phenyl}-((R)-3-methoxy-pyrrolidin-1-yl)-acetonitrile

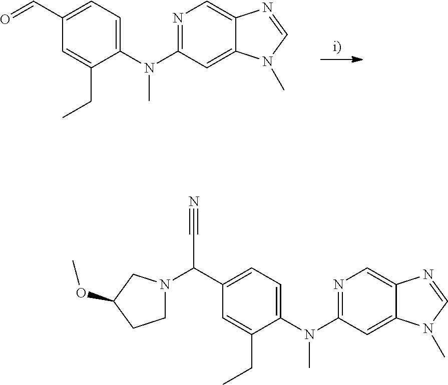 [SMILES: CCc1cc(C=O)ccc1N(C)c1cc2c(cn1)ncn2C ; CCc1cc(C(C#N)N2CC[C@@H](OC)C2)ccc1N(C)c1cc2c(cn1)ncn2C]

(R)-3-Methoxy-pyrrolidine hydrochloride (3 equiv, 70 mg) and DIPEA (3 equiv, 89 μL) were added to a mixture of the aldehyde (1 equiv, 50 mg) and LiClO₄ (2 equiv, 36 mg) in MeCN (0.9 mL). After stirring 5 min at room temperature, TMSCN (1.2 equiv, 28 μL) was added and the resulting mixture was stirred at room temperature for 18 h. After concentration, the residue was purified by silica chromatography (EtOAc; 100) to afford the desired compound.

### Compound 449: {3-Ethyl-4-[methyl-(1-methyl-1H-imidazo[4,5-c]pyridin-6-yl)-amino]-phenyl}-(4-methyl-piperazin-1-yl)-acetonitrile

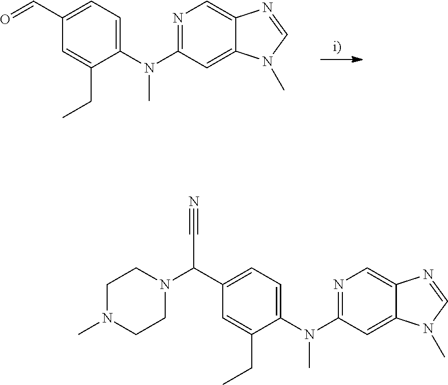 [SMILES: CCc1cc(C=O)ccc1N(C)c1cc2c(cn1)ncn2C ; CCc1cc(C(C#N)N2CCN(C)CC2)ccc1N(C)c1cc2c(cn1)ncn2C]

1-Methyl-piperazine (3 equiv, 45 μL) was added to a mixture of the aldehyde derivative (1 equiv, 40 mg) and LiClO₄ (2 equiv, 29 mg) in MeCN (0.7 mL). After stirring 5 min at room temperature, TMSCN (1.2 equiv, 22 μL) was added and the resulting mixture was stirred at room temperature for 18 h. After concentration, the residue was purified by silica chromatography (MeOH/DCM; 0:100 to 5:95) to afford the desired compound.

### Compound 450: {3-Ethyl-4-[methyl-(1-methyl-1H-imidazo[4,5-c]pyridin-6-yl)-amino]-phenyl}-(3-hydroxy-azetidin-1-yl)-acetonitrile

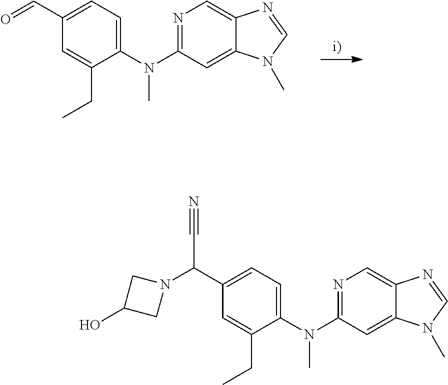 [SMILES: CCc1cc(C=O)ccc1N(C)c1cc2c(cn1)ncn2C ; CCc1cc(C(C#N)N2CC(O)C2)ccc1N(C)c1cc2c(cn1)ncn2C]

Azetidin-3-ol (3 equiv, 44 mg) and DIPEA (3 equiv, 71 μL) were added to a mixture of the aldehyde derivative (1 equiv, 40 mg) and LiClO₄ (2 equiv, 29 mg) in MeCN (0.7 mL). After stirring 5 min at room temperature, TMSCN (1.2 equiv, 22 μL) was added and the resulting mixture was stirred at room temperature for 18 h. After concentration, the residue was purified by silica chromatography (MeOH/DCM; 0:100 to 5:95) to afford the desired compound.

### Compound 451: 1-(Cyano-{3-ethyl-4-[methyl-(1-methyl-1H-imidazo[4,5-c]pyridin-6-yl)-amino]-phenyl}-methyl)-piperidine-4-carboxylic acid amide

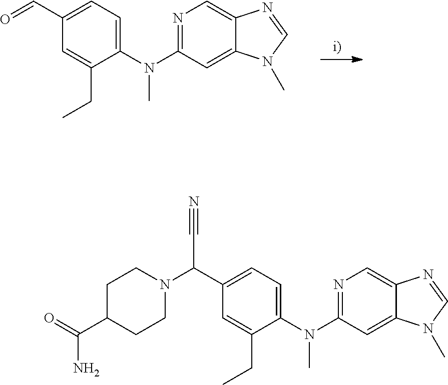 [SMILES: CCc1cc(C=O)ccc1N(C)c1cc2c(cn1)ncn2C ; CCc1cc(C(C#N)N2CCC(C(N)=O)CC2)ccc1N(C)c1cc2c(cn1)ncn2C]

Piperidine-4-carboxylic acid amide (3 equiv, 52 mg) was added to a mixture of the aldehyde derivative (1 equiv, 40 mg) and LiClO₄ (2 equiv, 29 mg) in MeCN (0.7 mL). After stirring 5 min at room temperature, TMSCN (1.2 equiv, 22 μL) was added and the resulting mixture was stirred at room temperature for 18 h. After concentration, the residue was purified by silica chromatography (MeOH/DCM; 0:100 to 5:95) to afford the desired compound.

### Compound 452: {3-Ethyl-4-[methyl-(1-methyl-1H-imidazo[4,5-c]pyridin-6-yl)-amino]-phenyl}-(4-methanesulfonyl-piperidin-1-yl)-acetonitrile

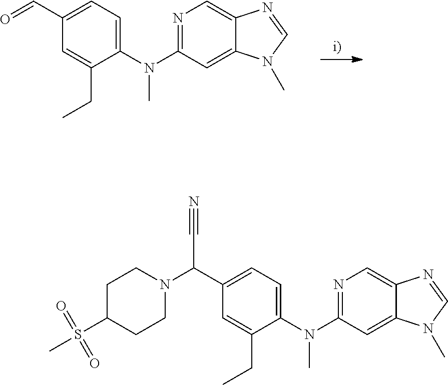 [SMILES: CCc1cc(C=O)ccc1N(C)c1cc2c(cn1)ncn2C ; CCc1cc(C(C#N)N2CCC(S(C)(=O)=O)CC2)ccc1N(C)c1cc2c(cn1)ncn2C]

4-Methanesulfonyl-piperidine (3 equiv, 67 mg) was added to a mixture of the aldehyde derivative (1 equiv, 40 mg) and LiClO₄ (2 equiv, 29 mg) in MeCN (0.7 mL). After stirring 5 min at room temperature, TMSCN (1.2 equiv, 22 μL) was added and the resulting mixture was stirred at room temperature for 18 h. After concentration, the residue was purified by silica chromatography (MeOH/DCM; 0:100 to 5:95) to afford the desired compound.

### Compound 453: 1-(Cyano-{3-ethyl-4-[methyl-(1-methyl-1H-imidazo[4,5-c]pyridin-6-yl)-amino]-phenyl}-methyl)-azetidine-3-carboxylic acid ethyl ester

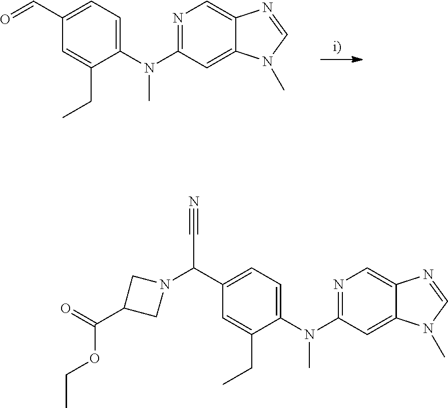 [SMILES: CCc1cc(C=O)ccc1N(C)c1cc2c(cn1)ncn2C ; CCOC(=O)C1CN(C(C#N)c2ccc(N(C)c3cc4c(cn3)ncn4C)c(CC)c2)C1]

Azetidine-3-carboxylic acid ethyl ester hydrochloride (3 equiv, 67 mg) and DIPEA (3 equiv, 71 μL) were added to a mixture of the aldehyde derivative (1 equiv, 40 mg) and LiClO₄ (2 equiv, 29 mg) in MeCN (0.7 mL). After stirring 5 min at room temperature, TMSCN (1.2 equiv, 22 μL) was added and the resulting mixture was stirred at room temperature for 18 h. After concentration, the residue was purified by silica chromatography (MeOH/DCM; 0:100 to 5:95) to afford the desired compound.

### Compound 454: N-[1-(Cyano-{3-ethyl-4-[methyl-(1-methyl-1H-imidazo[4,5-c]pyridin-6-yl)-amino]-phenyl}-methyl)-azetidin-3-yl]-propionamide

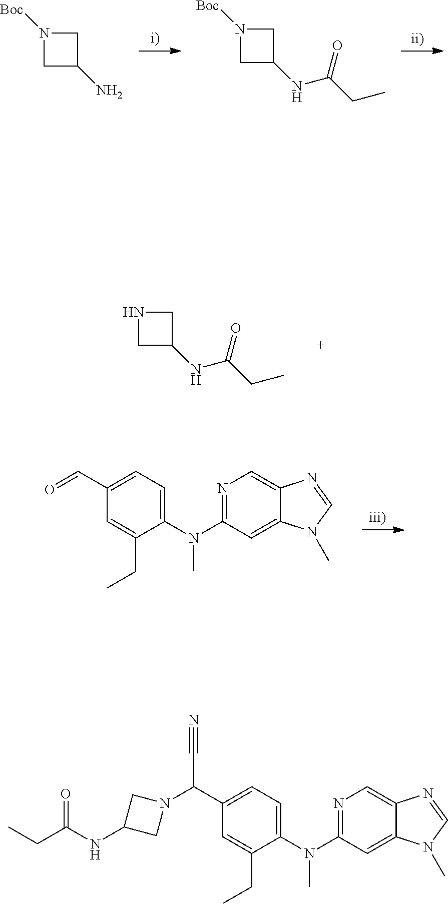

### Step (i): 3-Propionylamino-azetidine-1-carboxylic acid tert-butyl ester

Triethylamine (3 equiv, 0.4 mL) was added to a solution of 3-amino-azetidine-1-carboxylic acid tert-butyl ester (1 equiv, 166 mg) in DCM (10 mL). After 15 min, propionyl chloride (1.1 equiv, 92 μL) was added and the reaction mixture was stirred at room temperature for 16 h. Water was added and the resulting mixture was extracted with DCM (3×), dried and concentrated.

### Step (ii): N-Azetidin-3-yl-propionamide

Trifluoroacetic acide (0.7 mL) was added to a solution of the Boc-azetidine (1 equiv, 212 mg) in DCM (13 mL) and the reaction was stirred at room temperature for 16 h. After concentration, the residue was purified through a SCX column to lead the desired product.

### Step (iii): N-[7-(Cyano-{3-ethyl-4-[methyl-(1-methyl-1H-imidazo[4,5-c]pyridin-6-yl)-amino]-phenyl}-methyl)-azetidin-3-yl]-propionamide

The azetidine derivative (3 equiv, 56 mg) was added to a mixture of the aldehyde derivative (1 equiv, 43 mg) and LiClO₄ (2 equiv, 31 mg) in MeCN (0.7 mL). After stirring 5 min at room temperature, TMSCN (1.2 equiv, 24 μL) was added and the resulting mixture was stirred at room temperature for 18 h. After concentration, the residue was purified by silica chromatography (MeOH/DCM; 0:100 to 2:98) to afford the desired compound.

### Compound 455: N-[1-(Cyano-{3-ethyl-4-[methyl-(1-methyl-1H-imidazo[4,5-c]pyridin-6-yl)-amino]-phenyl}-methyl)-azetidin-3-yl]-methanesulfonamide

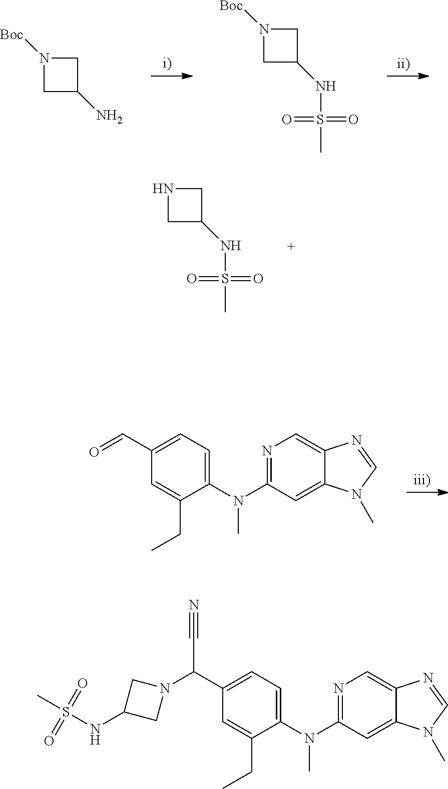

### Step (i): 3-Methanesulfonylamino-azetidine-1-carboxylic acid tert-butyl ester

Triethylamine (3 equiv, 0.4 mL) was added to a solution of 3-amino-azetidine-1-carboxylic acid tert-butyl ester (1 equiv, 167 mg) in DCM (10 mL). After 15 min, methanesulfonyl chloride (1.1 equiv, 83 μL) was added and the reaction mixture was stirred at room temperature for 16 h. Water was added and the resulting mixture was extracted with DCM (3×), dried and concentrated.

### Step (ii): N-Azetidin-3-yl-methanesulfonamide

Trifluoroacetic acid (0.7 mL) was added to a solution of the Boc-azetidine (1 equiv, 232 mg) in DCM (13 mL) and the reaction was stirred at room temperature for 16 h. After concentration, the residue was purified through a SCX column to lead the desired product.

### Step (iii): N-[1-(Cyano-{3-ethyl-4-[methyl-(1-methyl-1H-imidazo[4,5-c]pyridin-6-yl)-amino]-phenyl}-methyl)-azetidin-3-yl]-methanesulfonamide

The azetidine derivative (3 equiv, 98 mg) was added to a mixture of the aldehyde (1 equiv, 64 mg) and LiClO₄ (2 equiv, 46 mg) in MeCN (1.1 mL). After stirring 5 min at room temperature, TMSCN (1.2 equiv, 36 μL) was added and the resulting mixture was stirred at room temperature for 18 h. After concentration, the residue was purified by silica chromatography (MeOH/DCM; 0:100 to 2:98) to afford the desired compound.

### Compound 456: (4-Ethyl-pyrimidin-5-yl)-methyl-(1-methyl-1H-imidazo[4,5-c]pyridin-6-yl)-amine

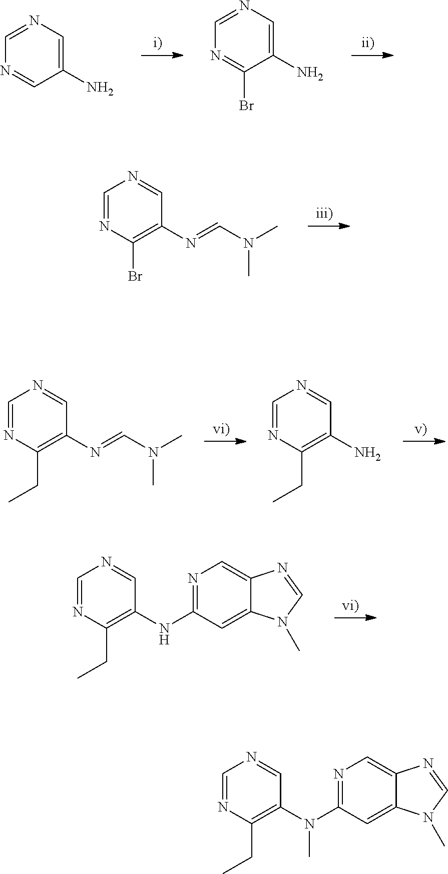

### Step (i): 4-Bromo-pyrimidin-5-ylamine

Benzyltrimethylammonium tribromide (1.1 equiv, 4.5 g) was added portionwised to a stirred solution of 5-aminopyrimidine (1 equiv, 1 g) in DCM (50 mL) and MeOH (10 mL) at 0° C. The mixture was warmed up to room temperature and stirred for 90 min. Aq. sat. NaHCO₃ was added (pH 8), the organic was separated and the aqueous extracted with EtOAc (3×). The combined organics were dried and concentrated. Purification by silica chromatography (EtOAc/cyclohexane; 0:100 to 50:50) gave the desired compound.

### Step (ii)

A solution of 4-bromo-pyrimidin-5-ylamine (1 equiv, 400 mg) and N,N-dimethylformamide dimethylacetal (1.6 mL) in MeOH (35 mL) was refluxed for 2 h. The mixture was concentrated to give the compound.

### Step (iii)

Diethylzinc (1M in hexanes, 2.5 equiv, 5.3 mL) was added to a degassed mixture of the bromoaryl (1 equiv, 480 mg), tri-o-tolylphosphine (0.61 equiv, 388 mg) and Pd(OAc)₂ (0.19 equiv, 89 mg) in DMF (8 mL) and the reaction was heated at 90° C. for 10 min. Water was then added and the resulting mixture was extracted with EtOAc (3×), dried and concentrated. Purification by silica chromatography (MeOH/DCM; 0:100 to 8:92) afforded the desired compound.

### Step (iv): 4-Ethyl-pyrimidin-5-ylamine

The dimethyl-formamidine (1 equiv, 178 mg) and K₂CO₃ (2.5 equiv, 345 g) in dioxane (15 mL) and water (15 mL) was heated at reflux for 72 h. The mixture was then extracted with EtOAc (3×), dried and concentrated. Purification by silica chromatography (MeOH/DCM; 0:100 to 10:90) led to the product.

### Step (v) and (vi): (4-Ethyl-pyrimidin-5-yl)-methyl-(1-methyl-1H-imidazo[4,5-c]pyridin-6-yl)-amine

Synthesis followed the same conditions used for compound 328 (step (ii)) and compound 385 (step (v)).

### Compound 457: 3-{6-Ethyl-5-[methyl-(1-methyl-1H-imidazo[4,5-c]pyridin-6-yl)-amino]-pyrazin-2-yl}-azetidine-1-carboxylic acid tert-butyl ester

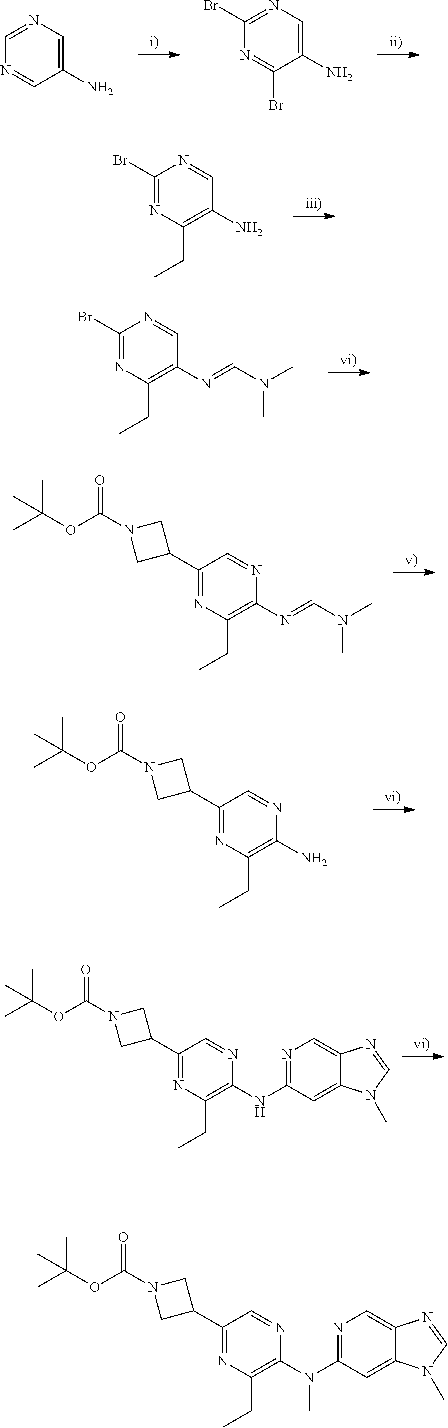

### Step (i): 2,4-Dibromo-pyrimidin-5-ylamine

N-bromo succinamide (2.1 equiv, 7.9 mg) was added to a solution of pyrazin-2-ylamine (1 equiv, 2 g) in DMSO (80 mL) and water (2 mL) at 0° C. After 1 h, the reaction was warmed to room temperature and stirred for 16 h. Water was added and the mixture extracted with EtOAc (4×), dried and concentrated. Purification by silica chromatography (EtOAc/cyclohexane; 0:100 to 50:50) afforded the desired compound.

### Step (ii): 2,4-Dibromo-pyrimidin-5-ylamine

Triethylborane (1M solution in THF, 1.01 equiv, 7.2 mL) was added to a mixture of the dibromoaryl (1 equiv, 1.8 g), Cs₂CO₃ (6 equiv, 13.9 g) and PdCl₂dppf (0.1 equiv, 581 mg) in DMF (36 mL) and the reaction mixture was stirred at 60° C. for 8 h. Water was added, the resulting mixture extracted with EtOAc (4×), dried and concentrated. Purification by silica chromatography (EtOAc/cyclohexane; 0:100 to 25:75) afforded the desired compound.

### Step (iii)

A solution of 4-bromo-3-ethyl-pyrazin-2-ylamine (1 equiv, 424 mg) and N,N-dimethylformamide dimethylacetal (1.45 mL) in MeOH (20 mL) was refluxed for 2 h. Water was added, the resulting mixture extracted with EtOAc (3×), dried and concentrated. Purification by silica chromatography (EtOAc/cyclohexane; 5:95 to 40:60) afforded the desired compound.

### Step (iv): 4-Ethyl-pyrimidin-5-ylamine

Iodine (0.2 equiv, 30 mg) was added to a degassed and dried suspension of zinc powder (3 equiv, 114 mg) and 3-iodo-azetidine-1-carboxylic acid tert-butyl ester (3 equiv, 493 mg) in dry DMA (1 mL) and the mixture was stirred at 90° C. for 6 h. This mixture was then cannulated to a solution of the bromoaryl (1 equiv, 147 mg), Pd(PPh₃)₄ (0.1 equiv, 40 mg) in DMA (1 mL) and the resulting mixture was stirred at 90° C. for 18 h. Water was added, the resulting mixture extracted with EtOAc (3×), dried and concentrated. Purification by silica chromatography (EtOAc/cyclohexane; 30:70 to 100:0) afforded the desired compound.

### Step (v): 3-(5-Amino-6-ethyl-pyrazin-2-yl)-azetidine-1-carboxylic acid tert-butyl ester

The dimethyl-formamidine (1 equiv, 102 mg) and K₂CO₃ (2.5 equiv, 103 g) in dioxane (5 mL) and water (5 mL) was heated at reflux for 24 h. The mixture was then extracted with EtOAc (3×), dried and concentrated. Purification by silica chromatography (EtOAc/cyclohexane; 15:75 to 100:0) led to the product.

### Step (vi) and (vii): 3-{6-Ethyl-5-[methyl-(1-methyl-1H-imidazo[4,5-c]pyridin-6-yl)-amino]-pyrazin-2-yl}-azetidine-1-carboxylic acid tert-butyl ester

Synthesis followed the same conditions used for compound 328 (step (ii)) and compound 385 (step (v)).

### Compound 458: 3-{4-Ethyl-5-[methyl-(1-methyl-1H-imidazo[4,5-c]pyridin-6-yl)-amino]-pyridin-2-yl}-1-methanesulfonyl-azetidine-3-carbonitrile

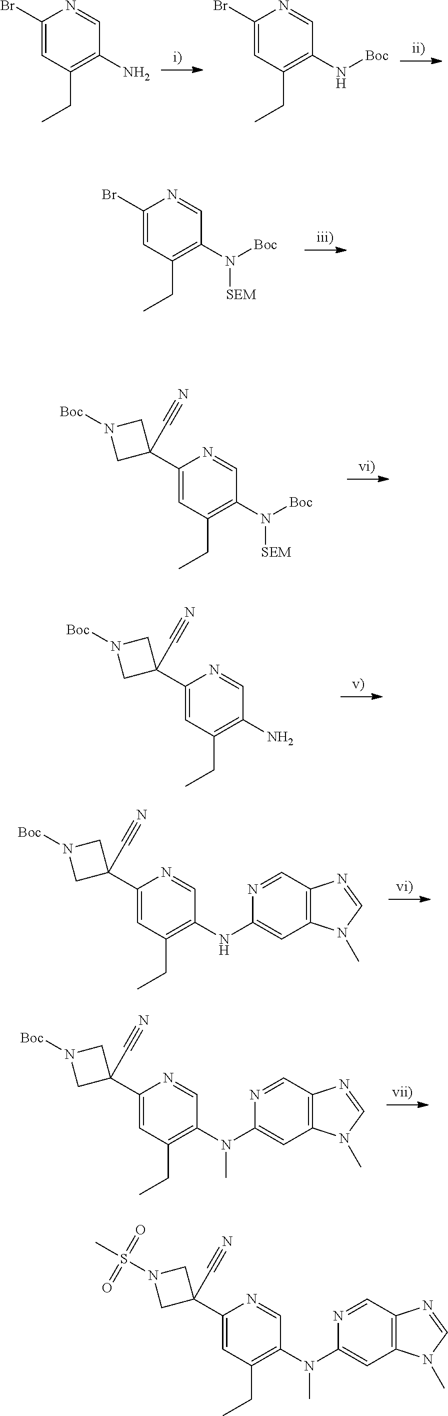

### Step (i): (6-Bromo-4-ethyl-pyridin-3-yl)-carbamic acid tert-butyl ester

NaHMDS (1M in THF, 2.1 equiv, 10.4 mL) was added dropwised to a solution of 6-bromo-4-ethyl-pyridin-3-ylamine (1 equiv, 1 g) and Boc₂O (1.1 equiv, 1.19 g) in dry THF at 0° C. The reaction was allowed to warm to room temperature and was stirred for 16 h. The reaction was quenched with aq. sat. NH₄Cl, extracted with EtOAc (3×). The combined organics was washed with brine, dried and concentrated. Purification by silica chromatography (EtOAc/cyclohexane; 10:90 to 70:30) led to the product.

### Step (ii): (6-Bromo-4-ethyl-pyridin-3-yl)-(2-trimethylsilanyl-ethoxymethyl)-carbamic acid tert-butyl ester

Sodium hydride (60%, 1.1 equiv, 167 mg) was added to a solution of the Boc-amine (1.1 equiv, 1.14 g) in DMF (20 mL) at 0° C. The mixture was warmed-up to room temperature and was stirred for 1 h, then SEMC1 (1.05 equiv, 0.7 mL) was added and the mixture was stirred at for 16 h. Water was added, the resulting mixture extracted with Et₂O (3×), dried and concentrated. Purification by silica chromatography (EtOAc/cyclohexane; 0:100 to 20:80) afforded the desired compound.

### Step (iii): 3-{5-[tert-Butoxycarbonyl-(2-trimethylsilanyl-ethoxymethyl)-amino]-4-ethyl-pyridin-2-yl}-3-cyano-azetidine-1-carboxylic acid tert-butyl ester

NaHMDS (1M in THF, 1.6 equiv, 3.2 mL) was added dropwised to a solution of the bromoaryl (1 equiv, 852 mg) and 3-cyano-azetidine-1-carboxylic acid tert-butyl ester (1 equiv, 360 mg) in toluene (4 mL) at 0° C. The mixture was warmed-up to room temperature and was stirred for 2 h. Water was added, the resulting mixture extracted with EtOAc (3×), dried and concentrated. Purification by silica chromatography (EtOAc/cyclohexane; 0:100 to 30:70) afforded the desired compound.

### Step (iv): 3-(5-Amino-4-ethyl-pyridin-2-yl)-3-cyano-azetidine-1-carboxylic acid tert-butyl ester

A solution of 4M HCl in 1,4-dioxane (10 mL) was added to a solution of 3-{5-[tert-Butoxycarbonyl-(2-trimethylsilanyl-ethoxymethyl)-amino]-4-ethyl-pyridin-2-yl}-3-cyano-azetidine-1-carboxylic acid tert-butyl ester (1 equiv, 600 mg) in dioxane (15 mL) and the mixture was stirred at room temperature for 2 h. After concentration, the residue was purified through SCX column and concentrated. To the purified compound in DCM (15 mL) was added NEt₃ (1.2 equiv, 188 μL) and a solution of Boc anhydride (1.05 equiv, 260 mg) in DCM (5 mL) and this reaction mixture was stirred at room temperature for 30 min. Water was added, the resulting mixture extracted with DCM (3×), dried and concentrated. Purification by silica chromatography (EtOAc/cyclohexane; 20:80 to 75:25) afforded the desired compound.

### Step (v) and (vi): 3-Cyano-3-{4-ethyl-5-[methyl-(1-methyl-1H-imidazo[4,5-c]pyridin-6-yl)-amino]-pyridin-2-yl}-azetidine-1-carboxylic acid tert-butyl ester

Synthesised following the same conditions used for compound 328 (step ii) and compound 385 (step v).

### Step (vii): 3-{4-Ethyl-5-[methyl-(1-methyl-1H-imidazo[4,5-c]pyridin-6-yl)-amino]-pyridin-2-yl}-1-methanesulfonyl-azetidine-3-carbonitrile

A solution of 4M HCl in dioxane (335 μL) was added to a solution of the boc-protected azetidine derivative (1 equiv, 40 mg) in 1,4-dioxane (3 mL) and the mixture was stirred at room temperature for 16 h. After concentration, the residue was purified through SCX column and concentrated To the purified compound redissolved in DCM (4 mL) was added pyridine (3 equiv, 21 μL) followed by methanesulfonyl chloride (1.2 equiv, 8.3 μL) and the resulting mixture was stirred at room temperature for 1 h. Water was added, the resulting mixture extracted with DCM (3×), dried and concentrated. Purification by silica chromatography (MeOH/DCM; 0:100 to 3.5:96.5) afforded the desired compound

A compound of the invention and the comparative examples that have been prepared according to the synthetic methods described herein are listed in Table I below. The NMR spectral data of the compounds of the invention and some of the comparative examples is given in Table II.

TABLE I
Compound of the invention.

| Cpd | | | | | MS |
|---|---|---|---|---|---|
| # | Structure | Name | Mtd | MW | Ms'd |
| 1 | 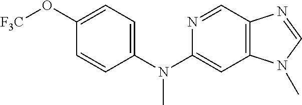 [SMILES: CN(c1ccc(OC(F)(F)F)cc1)c1cc2c(cn1)ncn2C] | N,1-dimethyl-N-(4- (trifluoromethoxy)phenyl)- 1H-imidazo[4,5- c]pyridin-6-amine | A | 322 | 322.9 |
| 2 | 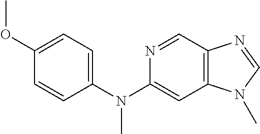 [SMILES: COc1ccc(N(C)c2cc3c(cn2)ncn3C)cc1] | N-(4-methoxyphenyl)- N,1-dimethyl-1H- imidazo[4,5-c]pyridin-6- amine | A | 268 | 269 |
| 3 | 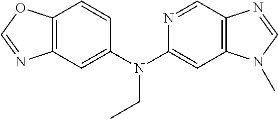 [SMILES: CCN(c1ccc2ocnc2c1)c1cc2c(cn1)ncn2C] | N-ethyl-N-(1-methyl-1H- imidazo[4,5-c]pyridin-6- yl)benzo[d]oxazol-5- amine | A′ and C | 293 | 294 |
| 4 | 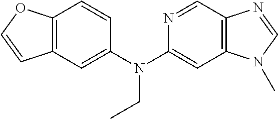 [SMILES: CCN(c1ccc2occc2c1)c1cc2c(cn1)ncn2C] | N-(benzofuran-5-yl)-N- ethyl-1-methyl-1H- imidazo[4,5-c]pyridin-6- amine | A′ and C | 292 | 293 |
| 5 | 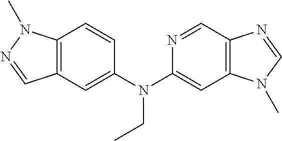 [SMILES: CCN(c1ccc2c(cnn2C)c1)c1cc2c(cn1)ncn2C] | N-ethyl-1-methyl-N-(1- methyl-1H-indazol-5-yl)- 1H-imidazo[4,5- c]pyridin-6-amine | A′ and C | 306 | 307 |
| 6 | 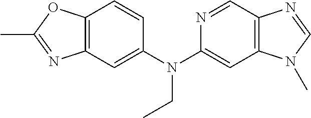 [SMILES: CCN(c1ccc2oc(C)nc2c1)c1cc2c(cn1)ncn2C] | N-ethyl-2-methyl-N-(1- methyl-1H-imidazo[4,5- c]pyridin-6- yl)benzo[d]oxazol-5- amine | A′ and C | 307 | 308 |
| 7 | 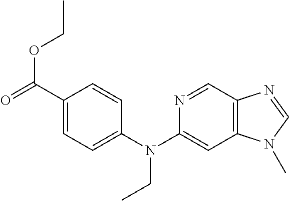 [SMILES: CCOC(=O)c1ccc(N(CC)c2cc3c(cn2)ncn3C)cc1] | ethyl 4-(ethyl(1-methyl- 1H-imidazo[4,5- c]pyridin-6- yl)amino)benzate | A′ and C | 324 | 325 |
| 8 | 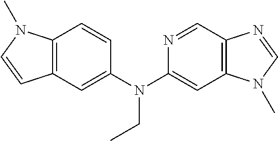 [SMILES: CCN(c1ccc2c(ccn2C)c1)c1cc2c(cn1)ncn2C] | N-ethyl-1-methyl-N-(1- methyl-1H-indol-5-yl)- 1H-imidazo[4,5- c]pyridin-6-amine | A′ and C | 305 | 306 |
| 9 | 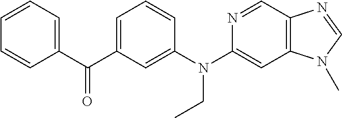 [SMILES: CCN(c1cccc(C(=O)c2ccccc2)c1)c1cc2c(cn1)ncn2C] | (3-(ethyl(1-methyl-1H- imidazo[4,5-c]pyridin-6- yl)amino)phenyl)(phenyl) methanone | A′ and C | 356 | 357 |
| 10 | 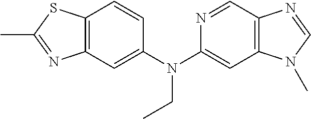 [SMILES: CCN(c1ccc2sc(C)nc2c1)c1cc2c(cn1)ncn2C] | N-ethyl-2-methyl-N-(1- methyl-1H-imidazo[4,5- c]pyridin-6- yl)benzo[d]thiazol-5- amine | A′ and C | 323 | 324 |
| 11 | 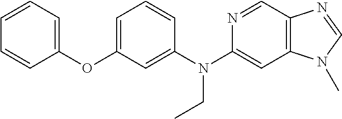 [SMILES: CCN(c1cccc(Oc2ccccc2)c1)c1cc2c(cn1)ncn2C] | N-ethyl-1-methyl-N-(3- phenoxyphenyl)-1H- imidazo[4,5-c]pyridin-6- amine | A′ and C | 344 | 345 |
| 12 | 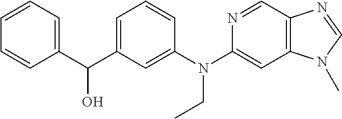 [SMILES: CCN(c1cccc(C(O)c2ccccc2)c1)c1cc2c(cn1)ncn2C] | (3-(ethyl(1-methyl-1H- imidazo[4,5-c]pyridin-6- yl)amino)phenyl)(phenyl) MeOH | A′ and C | 358 | 359 |
| 13 | 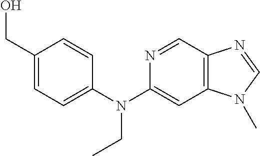 [SMILES: CCN(c1ccc(CO)cc1)c1cc2c(cn1)ncn2C] | (4-(ethyl(1-methyl-1H- imidazo[4,5-c]pyridin-6- yl)amino)phenyl)MeOH | Described above | 282 | 283 |
| 14 | 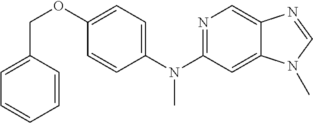 [SMILES: CN(c1ccc(OCc2ccccc2)cc1)c1cc2c(cn1)ncn2C] | N-(4-(benzyloxy)phenyl)- N,1-dimethyl-1H- imidazo[4,5-c]pyridin-6- amine | A′ and B | 344 | 345 |
| 15 | 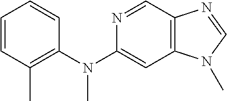 [SMILES: Cc1ccccc1N(C)c1cc2c(cn1)ncn2C] | N,1-dimethyl-N-o-tolyl 1H-imidazo[4,5- c]pyridin-6-amine | A and B | 252 | 253 |
| 16 | 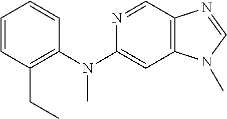 [SMILES: CCc1ccccc1N(C)c1cc2c(cn1)ncn2C] | N-(2-ethylphenyl)-N,1- dimethyl-1H-imidazo[4,5- c]pyridin-6-amine | Described above | 266 | 267 |
| 17 | 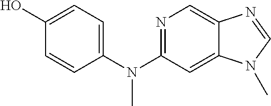 [SMILES: CN(c1ccc(O)cc1)c1cc2c(cn1)ncn2C] | 4-(methyl(1-methyl-1H- imidazo[4,5-c]pyridin-6- yl)amino)phenol | Described above | 254 | 255 |
| 18 | 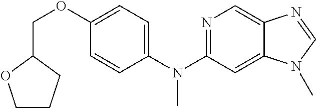 [SMILES: CN(c1ccc(OCC2CCCO2)cc1)c1cc2c(cn1)ncn2C] | N,1-dimethyl-N-(4- ((tetrahydrofuran-2- yl)methoxy)phenyl)-1H- imidazo[4,5-c]pyridin-6- amine | A′, B and F | 338 | 339 |
| 19 | 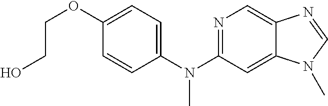 [SMILES: CN(c1ccc(OCCO)cc1)c1cc2c(cn1)ncn2C] | 2-(4-(methyl(1-methyl- 1H-imidazo[4,5- c]pyridin-6- yl)amino)phenoxy)ethanol | A, B and F | 298 | 299 |
| 20 | 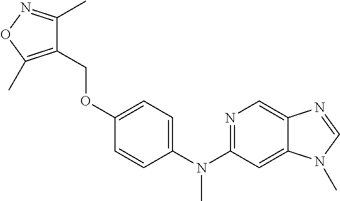 [SMILES: Cc1noc(C)c1COc1ccc(N(C)c2cc3c(cn2)ncn3C)cc1] | N-(4-((3,5- dimethylisoxazol-4- yl)methoxy)phenyl)-N,1- dimethyl-1H-imidazo[4,5- c]pyridin-6-amine | A, B and F | 363 | 364 |
| 21 | 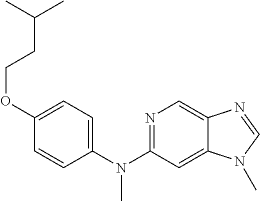 [SMILES: CC(C)CCOc1ccc(N(C)c2cc3c(cn2)ncn3C)cc1] | N-(4- (isopentyloxy)phenyl)- N,1-dimethyl-1H- imidazo[4,5-c]pyridin-6- amine | A′, B and F | 324 | 325 |
| 22 | 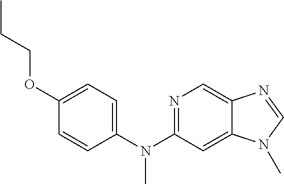 [SMILES: CCCOc1ccc(N(C)c2cc3c(cn2)ncn3C)cc1] | N,1-dimethyl-N-(4- propoxyphenyl)-1H- imidazo[4,5-c]pyridin-6- amine | A′, B and F | 296 | 297 |
| 23 | 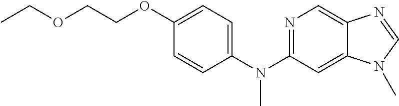 [SMILES: CCOCCOc1ccc(N(C)c2cc3c(cn2)ncn3C)cc1] | N-(4-(2- ethoxyethoxy)phenyl)- N,1-dimethyl-1H- imidazo[4,5-c]pyridin-6- amine | A′, B and F | 326 | 327 |
| 24 | 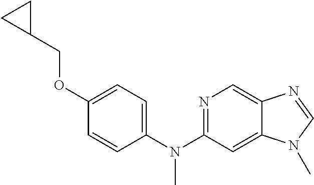 [SMILES: CN(c1ccc(OCC2CC2)cc1)c1cc2c(cn1)ncn2C] | N-(4- (cyclopropylmethoxy)phen- yl)-N,1-dimethyl-1H- imidazo[4,5-c]pyridin-6- amine | A′, B and F | 308 | 309 |
| 25 | 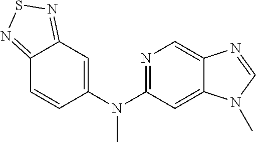 [SMILES: CN(c1ccc2nsnc2c1)c1cc2c(cn1)ncn2C] | N-methyl-N-(1-methyl- 1H-imidazo[4,5- c]pyridin-6- yl)benzo[c][1,2,5]thiadiazol- 5-amine | A′, B and F | 296 | 297 |
| 26 | 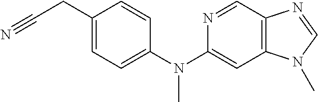 [SMILES: CN(c1ccc(CC#N)cc1)c1cc2c(cn1)ncn2C] | 2-(4-(methyl(1-methyl- 1H-imidazo[4,5- c]pyridin-6- yl)amino)phenyl)acetonitrile | Described above | 277 | 278 |
| 27 | 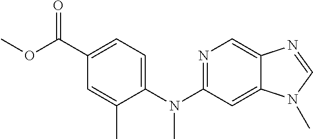 [SMILES: COC(=O)c1ccc(N(C)c2cc3c(cn2)ncn3C)c(C)c1] | methyl 3-methyl-4- (methyl(1-methyl-1H- imidazo[4,5-c]pyridin-6- yl)amino)benzate | Described above | 310 | 311 |
| 28 | 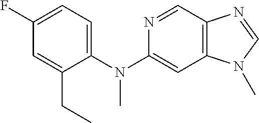 [SMILES: CCc1cc(F)ccc1N(C)c1cc2c(cn1)ncn2C] | N-(2-ethyl-4- fluorophenyl)-N,1- dimethyl-1H-imidazo[4,5- c]pyridin-6-amine | Described above | 284 | 285 |
| 29 | 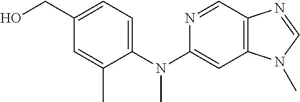 [SMILES: Cc1cc(CO)ccc1N(C)c1cc2c(cn1)ncn2C] | (3-methyl-4-(methyl(1- methyl-1H-imidazo[4,5- c]pyridin-6- yl)amino)phenyl)MeOH | Described above | 282 | 283 |
| 30 | 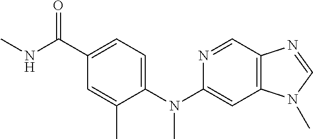 [SMILES: CNC(=O)c1ccc(N(C)c2cc3c(cn2)ncn3C)c(C)c1] | N,3-dimethyl-4- (methyl(1-methyl-1H- imidazo[4,5-c]pyridin-6- yl)amino)benzamide | Described above | 309 | 310 |
| 31 | 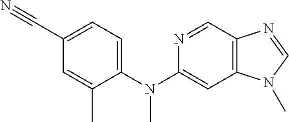 [SMILES: Cc1cc(C#N)ccc1N(C)c1cc2c(cn1)ncn2C] | 3-methyl-4-(methyl(1- methyl-1H-imidazo[4,5- c]pyridin-6- yl)amino)benzonitrile | Described above | 277 | 278 |
| 32 | 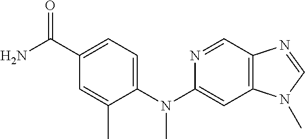 [SMILES: Cc1cc(C(N)=O)ccc1N(C)c1cc2c(cn1)ncn2C] | 3-methyl-4-(methyl(1- methyl-1H-imidazo[4,5- c]pyridin-6- yl)amino)benzamide | Described above | 295 | 296 |
| 33 | 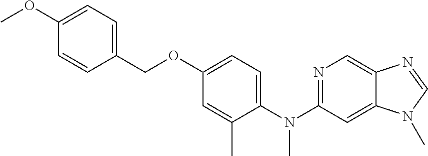 [SMILES: COc1ccc(COc2ccc(N(C)c3cc4c(cn3)ncn4C)c(C)c2)cc1] | N-(4-(4- methoxybenzyloxy)-2- methylphenyl)-N,1- dimethyl-1H-imidazo[4,5- c]pyridin-6-amine | Described above | 388 | 389 |
| 34 | 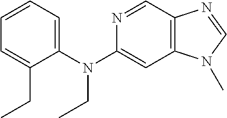 [SMILES: CCc1ccccc1N(CC)c1cc2c(cn1)ncn2C] | N-ethyl-N-(2- ethylphenyl)-1-methyl- 1H-imidazo[4,5- c]pyridin-6-amine | Described above | 280 | 281 |
| 35 | 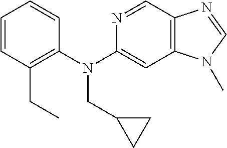 [SMILES: CCc1ccccc1N(CC1CC1)c1cc2c(cn1)ncn2C] | N-(cyclopropylmethyl)- N-(2-ethylphenyl)-1- methyl-1H-imidazo[4,5- c]pyridin-6-amine | A and E | 306 | 307 |
| 36 | 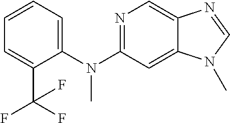 [SMILES: CN(c1cc2c(cn1)ncn2C)c1ccccc1C(F)(F)F] | N,1-dimethyl-N-(2- (trifluoromethyl)phenyl)- 1H-imidazo[4,5- c]pyridin-6-amine | A″ and B | 306 | 307 |
| 37 | 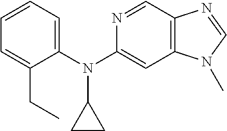 [SMILES: CCc1ccccc1N(c1cc2c(cn1)ncn2C)C1CC1] | N-cyclopropyl-N-(2- ethylphenyl)-1-methyl- 1H-imidazo[4,5- c]pyridin-6-amine | Described above | 292 | 293 |
| 38 | 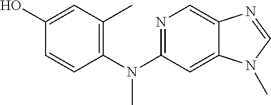 [SMILES: Cc1cc(O)ccc1N(C)c1cc2c(cn1)ncn2C] | 3-methyl-4-(methyl(1- methyl-1H-imidazo[4,5- c]pyridin-6- yl)amino)phenol | Described above | 268 | 269 |
| 39 | 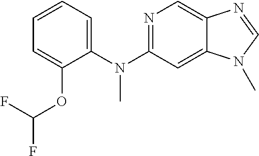 [SMILES: CN(c1cc2c(cn1)ncn2C)c1ccccc1OC(F)F] | N-(2- (difluoromethoxy)phenyl)- N,1-dimethyl-1H- imidazo[4,5-c]pyridin-6- amine | A″ and B | 304 | 305 |
| 40 | 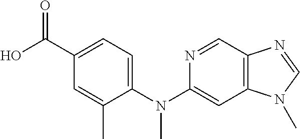 [SMILES: Cc1cc(C(=O)O)ccc1N(C)c1cc2c(cn1)ncn2C] | 3-methyl-4-(methyl(1- methyl-1H-imidazo[4,5- c]pyridin-6- yl)amino)benzic acid | Described above | 296 | 297 |
| 41 | 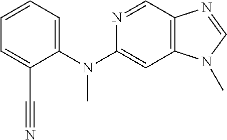 [SMILES: CN(c1cc2c(cn1)ncn2C)c1ccccc1C#N] | 2-(methyl(1-methyl-1H- imidazo[4,5-c]pyridin-6- yl)amino)benzonitrile | Described above | 263 | 264 |
| 42 | 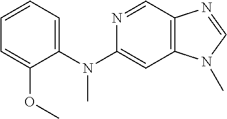 [SMILES: COc1ccccc1N(C)c1cc2c(cn1)ncn2C] | N-(2-methoxyphenyl)- N,1-dimethyl-1H- imidazo[4,5-c]pyridin-6- amine | Same as 60 | 268 | 269 |
| 43 | 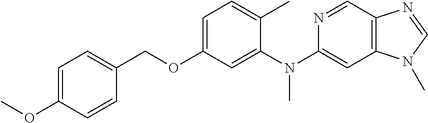 [SMILES: COc1ccc(COc2ccc(C)c(N(C)c3cc4c(cn3)ncn4C)c2)cc1] | N-(5-(4- methoxybenzyloxy)-2- methylphenyl)-N,1- dimethyl-1H-imidazo[4,5- c]pyridin-6-amine | Same as 33 | 388 | 389 |
| 44 | 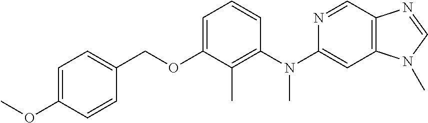 [SMILES: COc1ccc(COc2cccc(N(C)c3cc4c(cn3)ncn4C)c2C)cc1] | N-(3-(4- methoxybenzyloxy)-2- methylphenyl)-N,1- dimethyl-1H-imidazo[4,5- c]pyridin-6-amine | Same as 33 | 388 | 389 |
| 45 | 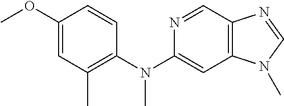 [SMILES: COc1ccc(N(C)c2cc3c(cn2)ncn3C)c(C)c1] | N-(4-methoxy-2- methylphenyl)-N,1- dimethyl-1H-imidazo[4,5- c]pyridin-6-amine | Described above | 282 | 283 |
| 46 | 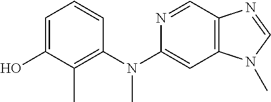 [SMILES: Cc1c(O)cccc1N(C)c1cc2c(cn1)ncn2C] | 2-methyl-3-(methyl(1- methyl-1H-imidazo[4,5- c]pyridin-6- yl)amino)phenol | Same as 38 | 268 | 269 |
| 47 | 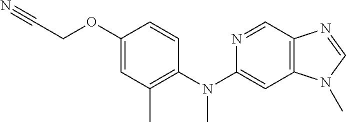 [SMILES: Cc1cc(OCC#N)ccc1N(C)c1cc2c(cn1)ncn2C] | 2-(3-methyl-4-(methyl(1- methyl-1H-imidazo[4,5- c]pyridin-6- yl)amino)phenoxy)acetoni- trile | Same as 45 | 307 | 308 |
| 48 | 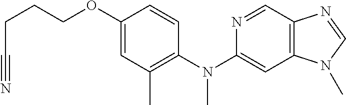 [SMILES: Cc1cc(OCCCC#N)ccc1N(C)c1cc2c(cn1)ncn2C] | 4-(3-methyl-4-(methyl(1- methyl-1H-imidazo[4,5- c]pyridin-6- yl)amino)phenoxy)butane nitrile | Same as 45 | 335 | 336 |
| 49 | 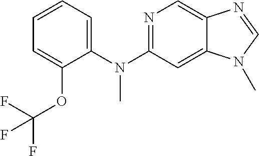 [SMILES: CN(c1cc2c(cn1)ncn2C)c1ccccc1OC(F)(F)F] | N,1-dimethyl-N-(2- (trifluoromethoxy)phenyl)- 1H-imidazo[4,5- c]pyridin-6-amine | A″ and B | 322 | 323 |
| 50 | 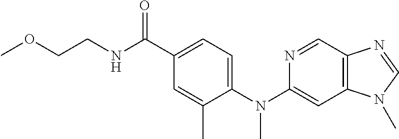 [SMILES: COCCNC(=O)c1ccc(N(C)c2cc3c(cn2)ncn3C)c(C)c1] | N-(2-methoxyethyl)-3- methyl-4-(methyl(1- methyl-1H-imidazo[4,5- c]pyridin-6- yl)amino)benzamide | Described above | 353 | 354 |
| 51 | 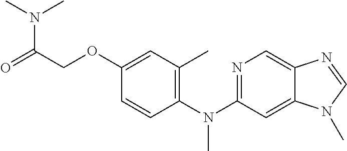 [SMILES: Cc1cc(OCC(=O)N(C)C)ccc1N(C)c1cc2c(cn1)ncn2C] | N,N-dimethyl-2-(3- methyl-4-(methyl(1- methyl-1H-imidazo[4,5- c]pyridin-6- yl)amino)phenoxy)acetamide | Same as 45 | 353 | 354 |
| 52 | 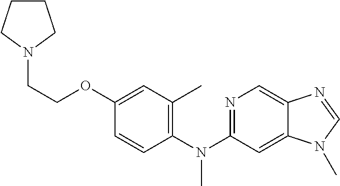 [SMILES: Cc1cc(OCCN2CCCC2)ccc1N(C)c1cc2c(cn1)ncn2C] | N,1-dimethyl-N-(2- methyl-4-(2-(pyrrolidin-1- yl)ethoxy)phenyl)-1H- imidazo[4,5-c]pyridin-6- amine | Same as 45 | 365 | 366 |
| 53 | 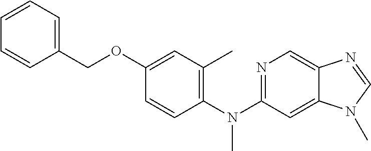 [SMILES: Cc1cc(OCc2ccccc2)ccc1N(C)c1cc2c(cn1)ncn2C] | N-(4-(benzyloxy)-2- methylphenyl)-N,1- dimethyl-1H-imidazo[4,5- c]pyridin-6-amine | Same as 45 | 358 | 359 |
| 54 | 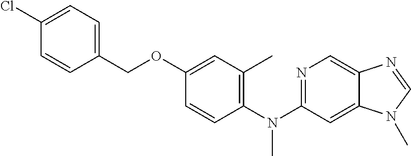 [SMILES: Cc1cc(OCc2ccc(Cl)cc2)ccc1N(C)c1cc2c(cn1)ncn2C] | N-(4-(4- chlorobenzyloxy)-2- methylphenyl)-N,1- dimethyl-1H-imidazo[4,5- c]pyridin-6-amine | Same as 45 | 393 | 393 |
| 55 | 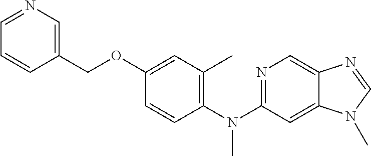 [SMILES: Cc1cc(OCc2cccnc2)ccc1N(C)c1cc2c(cn1)ncn2C] | N,1-dimethyl-N-(2- methyl-4-(pyridin-3- ylmethoxy)phenyl)-1H- imidazo[4,5-c]pyridin-6- amine | Same as 45 | 359 | 360 |
| 56 | 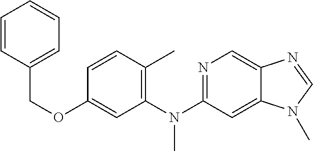 [SMILES: Cc1ccc(OCc2ccccc2)cc1N(C)c1cc2c(cn1)ncn2C] | N-(5-(benzyloxy)-2- methylphenyl)-N,1- dimethyl-1H-imidazo[4,5- c]pyridin-6-amine | Same as 45 | 358 | 359 |
| 57 | 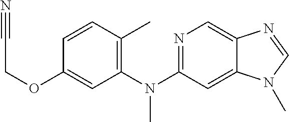 [SMILES: Cc1ccc(OCC#N)cc1N(C)c1cc2c(cn1)ncn2C] | 2-(4-methyl-3-(methyl(1- methyl-1H-imidazo[4,5- c]pyridin-6- yl)amino)phenoxy)acetoni- trile | Same as 45 | 307 | 308 |
| 58 | 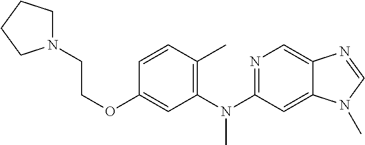 [SMILES: Cc1ccc(OCCN2CCCC2)cc1N(C)c1cc2c(cn1)ncn2C] | N,1-dimethyl-N-(2- methyl-5-(2-(pyrrolidin-1- yl)ethoxy)phenyl)-1H- imidazo[4,5-c]pyridin-6- amine | Same as 45 | 365 | 466 |
| 59 | 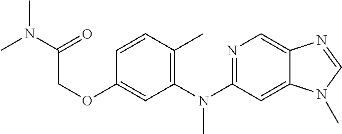 [SMILES: Cc1ccc(OCC(=O)N(C)C)cc1N(C)c1cc2c(cn1)ncn2C] | N,N-dimethyl-2-(4- methyl-3-(methyl(1- methyl-1H-imidazo[4,5- c]pyridin-6- yl)amino)phenoxy)acetamide | Same as 45 | 353 | 354 |
| 60 | 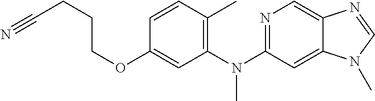 [SMILES: Cc1ccc(OCCCC#N)cc1N(C)c1cc2c(cn1)ncn2C] | 4-(4-methyl-3-(methyl(1- methyl-1H-imidazo[4,5- c]pyridin-6- yl)amino)phenoxy)butane nitrile | Same as 45 | 335 | 336 |
| 61 | 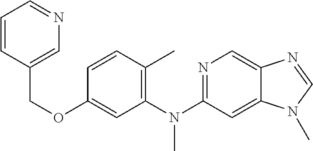 [SMILES: Cc1ccc(OCc2cccnc2)cc1N(C)c1cc2c(cn1)ncn2C] | N,1-dimethyl-N-(2- methyl-5-(pyridin-3- ylmethoxy)phenyl)-1H- imidazo[4,5-c]pyridin-6- amine | Same as 45 | 359 | 360 |
| 62 | 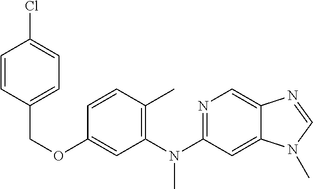 [SMILES: Cc1ccc(OCc2ccc(Cl)cc2)cc1N(C)c1cc2c(cn1)ncn2C] | N-(5-(4- chlorobenzyloxy)-2- methylphenyl)-N,1- dimethyl-1H-imidazo[4,5- c]pyridin-6-amine | Same as 45 | 393 | 393 |
| 63 | 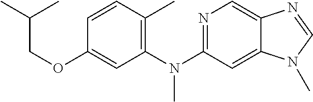 [SMILES: Cc1ccc(OCC(C)C)cc1N(C)c1cc2c(cn1)ncn2C] | N-(5-isobutoxy-2- methylphenyl)-N,1- dimethyl-1H-imidazo[4,5- c]pyridin-6-amine | Same as 45 | 324 | 325 |
| 64 | 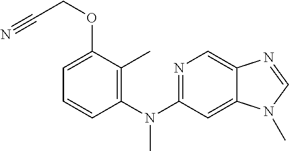 [SMILES: Cc1c(OCC#N)cccc1N(C)c1cc2c(cn1)ncn2C] | 2-(2-methyl-3-(methyl(1- methyl-1H-imidazo[4,5- c]pyridin-6- yl)amino)phenoxy)acetoni- trile | Same as 45 | 307 | 308 |
| 65 | 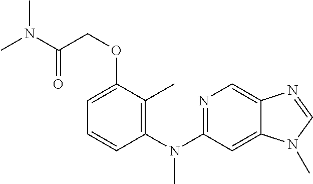 [SMILES: Cc1c(OCC(=O)N(C)C)cccc1N(C)c1cc2c(cn1)ncn2C] | N,N-dimethyl-2-(2- methyl-3-(methyl(1- methyl-1H-imidazo[4,5- c]pyridin-6- yl)amino)phenoxy)acetamide | Same as 45 | 353 | 354 |
| 66 | 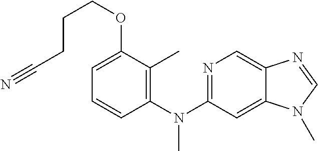 [SMILES: Cc1c(OCCCC#N)cccc1N(C)c1cc2c(cn1)ncn2C] | 4-(2-methyl-3-(methyl(1- methyl-1H-imidazo[4,5- c]pyridin-6- yl)amino)phenoxy)butane nitrile | Same as 45 | 335 | 336 |
| 67 | 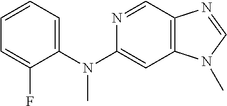 [SMILES: CN(c1cc2c(cn1)ncn2C)c1ccccc1F] | N-(2-fluorophenyl)-N,1- dimethyl-1H-imidazo[4,5- c]pyridin-6-amine | Same as 41 | 256 | 257 |
| 68 | 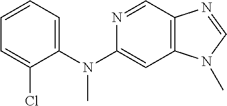 [SMILES: CN(c1cc2c(cn1)ncn2C)c1ccccc1Cl] | N-(2-chlorophenyl)-N,1- dimethyl-1H-imidazo[4,5- c]pyridin-6-amine | Described above | 273 | 273 |
| 69 | 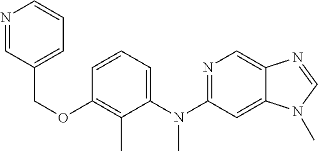 [SMILES: Cc1c(OCc2cccnc2)cccc1N(C)c1cc2c(cn1)ncn2C] | N,1-dimethyl-N-(2- methyl-3-(pyridin-3- ylmethoxy)phenyl)-1H- imidazo[4,5-c]pyridin-6- amine | Same as 45 | 359 | 360 |
| 70 | 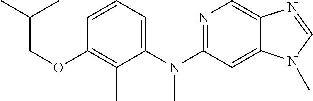 [SMILES: Cc1c(OCC(C)C)cccc1N(C)c1cc2c(cn1)ncn2C] | N-(3-isobutoxy-2- methylphenyl)-N,1- dimethyl-1H-imidazo[4,5- c]pyridin-6-amine | Same as 45 | 324 | 325 |
| 71 | 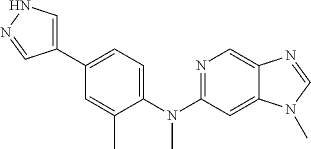 [SMILES: Cc1cc(-c2cn[nH]c2)ccc1N(C)c1cc2c(cn1)ncn2C] | N,1-dimethyl-N-(2- methyl-4-(1H-pyrazol-4- yl)phenyl)-1H- imidazo[4,5-c]pyridin-6- amine | Described above | 318 | 319 |
| 72 | 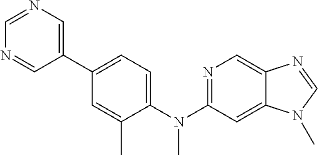 [SMILES: Cc1cc(-c2cncnc2)ccc1N(C)c1cc2c(cn1)ncn2C] | N,1-dimethyl-N-(2- methyl-4-(pyrimidin-5- yl)phenyl)-1H- imidazo[4,5-c]pyridin-6- amine | Same as 71 | 330 | 331 |
| 73 | 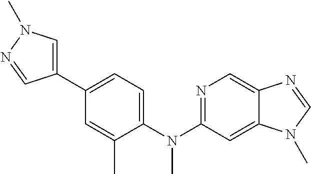 [SMILES: Cc1cc(-c2cnn(C)c2)ccc1N(C)c1cc2c(cn1)ncn2C] | N,1-dimethyl-N-(2- methyl-4-(1-methyl-1H- pyrazol-4-yl)phenyl)-1H- imidazo[4,5-c]pyridin-6- amine | Same as 71 | 332 | 333 |
| 74 | 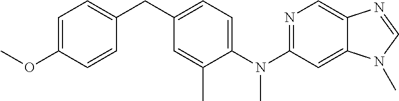 [SMILES: COc1ccc(Cc2ccc(N(C)c3cc4c(cn3)ncn4C)c(C)c2)cc1] | N-(4-(4-methoxybenzyl)- 2-methylphenyl)-N,1- dimethyl-1H-imidazo[4,5- c]pyridin-6-amine | Described above | 372 | 373 |
| 75 | 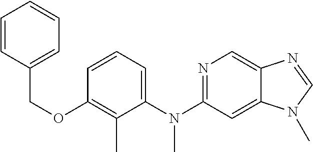 [SMILES: Cc1c(OCc2ccccc2)cccc1N(C)c1cc2c(cn1)ncn2C] | N-(3-(benzyloxy)-2- methylphenyl)-N,1- dimethyl-1H-imidazo[4,5- c]pyridin-6-amine | Same as 45 | 358 | 359 |
| 76 | 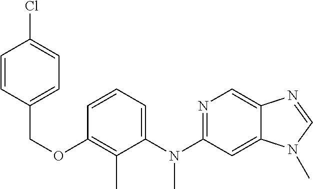 [SMILES: Cc1c(OCc2ccc(Cl)cc2)cccc1N(C)c1cc2c(cn1)ncn2C] | N-(3-(4- chlorobenzyloxy)-2- ethylphenyl)-N,1- dimethyl-1H-imidazo[4,5- c]pyridin-6-amine | Same as 45 | 393 | 393 |
| 77 | 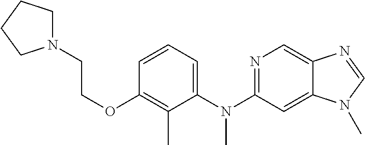 [SMILES: Cc1c(OCCN2CCCC2)cccc1N(C)c1cc2c(cn1)ncn2C] | N,1-dimethyl-N-(2- methyl-3-(2-(pyrrolidin-1- yl)ethoxy)phenyl)-1H- imidazo[4,5-c]pyridin-6- amine | Same as 45 | 365 | 366 |
| 78 | 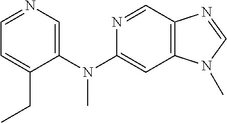 [SMILES: CCc1ccncc1N(C)c1cc2c(cn1)ncn2C] | N-(4-ethylpyridin-3-yl)- N,1-dimethyl-1H- imidazo[4,5-c]pyridin-6- amine | A″ and B using inter- mediate 3 | 267 | 268 |
| 79 | 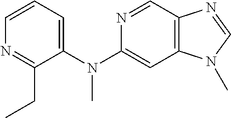 [SMILES: CCc1ncccc1N(C)c1cc2c(cn1)ncn2C] | N-(2-ethylpyridin-3-yl)- N,1-dimethyl-1H- imidazo[4,5-c]pyridin-6- amine | A″ and B using inter- mediate 3 | 267 | 268 |
| 80 | 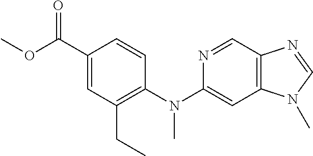 [SMILES: CCc1cc(C(=O)OC)ccc1N(C)c1cc2c(cn1)ncn2C] | methyl 3-ethyl-4- (methyl(1-methyl-1H- imidazo[4,5-c]pyridin-6- yl)amino)benzate | Described above | 324 | 325 |
| 81 | 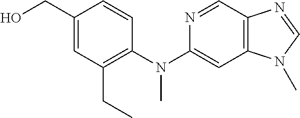 [SMILES: CCc1cc(CO)ccc1N(C)c1cc2c(cn1)ncn2C] | (3-ethyl-4-(methyl(1- methyl-1H-imidazo[4,5- c]pyridin-6- yl)amino)phenyl)MeOH | Described above | 296 | 297 |
| 82 | 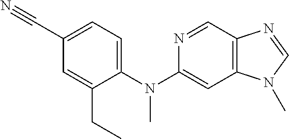 [SMILES: CCc1cc(C#N)ccc1N(C)c1cc2c(cn1)ncn2C] | 3-ethyl-4-(methyl(1- methyl-1H-imidazo[4,5- c]pyridin-6- yl)amino)benzonitrile | A″ and B | 291 | 292 |
| 83 | 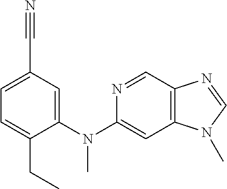 [SMILES: CCc1ccc(C#N)cc1N(C)c1cc2c(cn1)ncn2C] | 4-ethyl-3-(methyl(1- methyl-1H-imidazo[4,5- c]pyridin-6- yl)amino)benzonitrile | Same as compound 28 | 291 | 292 |
| 84 | 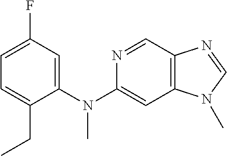 [SMILES: CCc1ccc(F)cc1N(C)c1cc2c(cn1)ncn2C] | N-(2-ethyl-5- fluorophenyl)-N,1- dimethyl-1H-imidazo[4,5- c]pyridin-6-amine | Same as compound 28 | 284 | 285 |
| 85 | 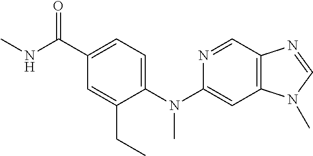 [SMILES: CCc1cc(C(=O)NC)ccc1N(C)c1cc2c(cn1)ncn2C] | 3-ethyl-N-methyl-4- (methyl(1-methyl-1H- imidazo[4,5-c]pyridin-6- yl)amino)benzamide | Described above | 323 | 324 |
| 86 | 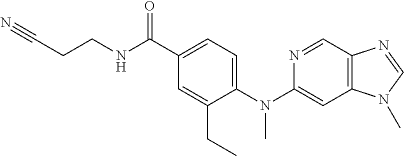 [SMILES: CCc1cc(C(=O)NCCC#N)ccc1N(C)c1cc2c(cn1)ncn2C] | N-(2-cyanoethyl)-3-ethyl- 4-(methyl(1-methyl-1H- imidazo[4,5-c]pyridin-6- yl)amino)benzamide | Described above | 362 | 363 |
| 87 | 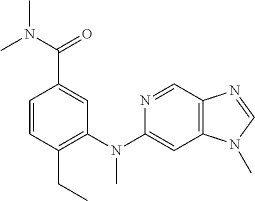 [SMILES: CCc1ccc(C(=O)N(C)C)cc1N(C)c1cc2c(cn1)ncn2C] | 4-ethyl-N,N-dimethyl-3- (methyl(1-methyl-1H- imidazo[4,5-c]pyridin-6- yl)amino)benzamide | Described above | 337 | 338 |
| 88 | 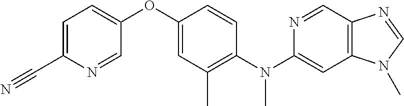 [SMILES: Cc1cc(Oc2ccc(C#N)nc2)ccc1N(C)c1cc2c(cn1)ncn2C] | 5-(3-methyl-4-(methyl(1- methyl-1H-imidazo[4,5- c]pyridin-6- yl)amino)phenoxy)picolino- nitrile | Method I | 370 | 371 |
| 89 | 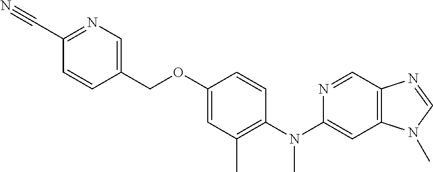 [SMILES: Cc1cc(OCc2ccc(C#N)nc2)ccc1N(C)c1cc2c(cn1)ncn2C] | 5-((3-methyl-4-(methyl(1- methyl-1H-imidazo[4,5- c]pyridin-6- yl)amino)phenoxy)methyl) picolinonitrile | Same as compound 45 | 384 | 385 |
| 90 | 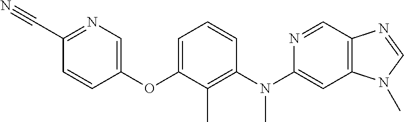 [SMILES: Cc1c(Oc2ccc(C#N)nc2)cccc1N(C)c1cc2c(cn1)ncn2C] | 5-(2-methyl-3-(methyl(1- methyl-1H-imidazo[4,5- c]pyridin-6- yl)amino)phenoxy)picolino- nitrile | I | 370 | 371 |
| 91 | 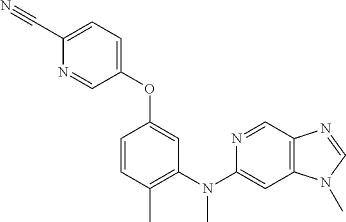 [SMILES: Cc1ccc(Oc2ccc(C#N)nc2)cc1N(C)c1cc2c(cn1)ncn2C] | 5-(4-methyl-3-(methyl(1- methyl-1H-imidazo[4,5- c]pyridin-6- yl)amino)phenoxy)picolino- nitrile | I | 370 | 371 |
| 92 | 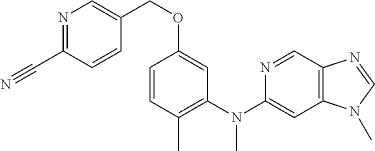 [SMILES: Cc1ccc(OCc2ccc(C#N)nc2)cc1N(C)c1cc2c(cn1)ncn2C] | 5-((4-methyl-3-(methyl(1- methyl-1H-imidazo[4,5- c]pyridin-6- yl)amino)phenoxy)methyl) picolinonitrile | Same as 45 | 384 | 385 |
| 93 | 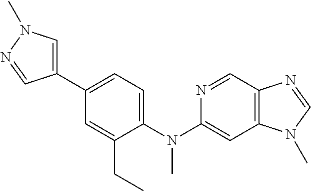 [SMILES: CCc1cc(-c2cnn(C)c2)ccc1N(C)c1cc2c(cn1)ncn2C] | N-(2-ethyl-4-(1-methyl- 1H-pyrazol-4-yl)phenyl)- N,1-dimethyl-1H- imidazo[4,5-c]pyridin-6- amine | Described above | 346 | 347 |
| 94 | 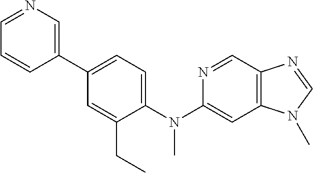 [SMILES: CCc1cc(-c2cccnc2)ccc1N(C)c1cc2c(cn1)ncn2C] | N-(2-ethyl-4-(pyridin-3- yl)phenyl)-N,1-dimethyl- 1H-imidazo[4,5- c]pyridin-6-amine | G | 343 | 344 |
| 95 | 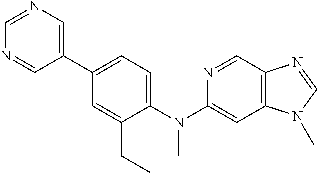 [SMILES: CCc1cc(-c2cncnc2)ccc1N(C)c1cc2c(cn1)ncn2C] | N-(2-ethyl-4-(pyrimidin- 5-yl)phenyl)-N,1- dimethyl-1H-imidazo[4,5- c]pyridin-6-amine | G | 344 | 345 |
| 96 | 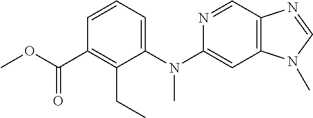 [SMILES: CCc1c(C(=O)OC)cccc1N(C)c1cc2c(cn1)ncn2C] | methyl 2-ethyl-3- (methyl(1-methyl-1H- imidazo[4,5-c]pyridin-6- yl)amino)benzate | A″ and B | 324 | 325 |
| 97 | 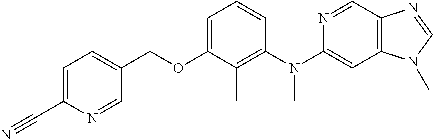 [SMILES: Cc1c(OCc2ccc(C#N)nc2)cccc1N(C)c1cc2c(cn1)ncn2C] | 5-((2-methyl-3-(methyl(1- methyl-1H-imidazo[4,5- c]pyridin-6- yl)amino)phenoxy)methyl) picolinonitrile | Same as 45 | 384 | 385 |
| 98 | 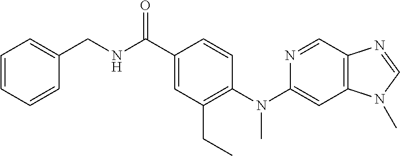 [SMILES: CCc1cc(C(=O)NCc2ccccc2)ccc1N(C)c1cc2c(cn1)ncn2C] | N-benzyl-3-ethyl-4- (methyl(1-methyl-1H- imidazo[4,5-c]pyridin-6- yl)amino)benzamide | Same as 86 | 399 | 400 |
| 99 | 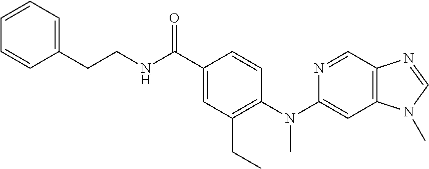 [SMILES: CCc1cc(C(=O)NCCc2ccccc2)ccc1N(C)c1cc2c(cn1)ncn2C] | 3-ethyl-4-(methyl(1- methyl-1H-imidazo[4,5- c]pyridin-6-yl)amino)-N- phenethylbenzamide | Same as 86 | 414 | 414 |
| 100 | 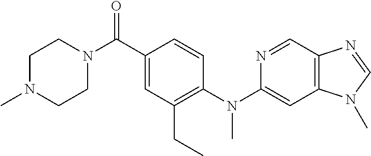 [SMILES: CCc1cc(C(=O)N2CCN(C)CC2)ccc1N(C)c1cc2c(cn1)ncn2C] | (3-ethyl-4-(methyl(1- methyl-1H-imidazo[4,5- c]pyridin-6- yl)amino)phenyl)(4- methylpiperazin-1- yl)methanone | H | 393 | 393 |
| 101 | 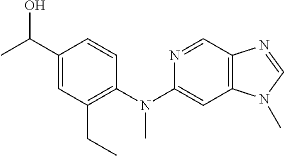 [SMILES: CCc1cc(C(C)O)ccc1N(C)c1cc2c(cn1)ncn2C] | 1-(3-ethyl-4-(methyl(1- methyl-1H-imidazo[4,5- c]pyridin-6- yl)amino)phenyl)ethanol | Described above | 310 | 311 |
| 102 | 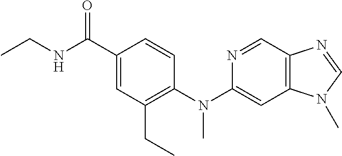 [SMILES: CCNC(=O)c1ccc(N(C)c2cc3c(cn2)ncn3C)c(CC)c1] | N,3-diethyl-4-(methyl(1- methyl-1H-imidazo[4,5- c]pyridin-6- yl)amino)benzamide | H | 337 | 338 |
| 103 | 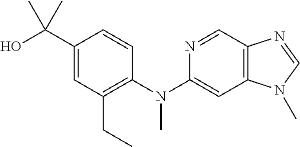 [SMILES: CCc1cc(C(C)(C)O)ccc1N(C)c1cc2c(cn1)ncn2C] | 2-(3-ethyl-4-(methyl(1- methyl-1H-imidazo[4,5- c]pyridin-6- yl)amino)phenyl)propan- 2-ol | Described above | 324 | 325 |
| 104 | 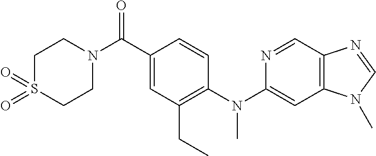 [SMILES: CCc1cc(C(=O)N2CCS(=O)(=O)CC2)ccc1N(C)c1cc2c(cn1)ncn2C] | (1,1-Dioxothiomorpholin- 4-yl)-{3-ethyl-4-[methyl- (1-methyl-1H- imidazo[4,5-c]pyridin-6- yl)-amino]-phenyl}- methanone | H | 428 | 428 |
| 105 | 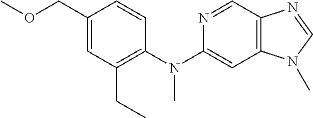 [SMILES: CCc1cc(COC)ccc1N(C)c1cc2c(cn1)ncn2C] | N-(2-ethyl-4- (methoxymethyl)phenyl)- N,1-dimethyl-1H- imidazo[4,5-c]pyridin-6- amine | Described above | 310 | 311 |
| 106 | 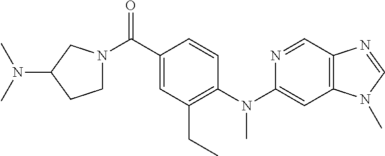 [SMILES: CCc1cc(C(=O)N2CCC(N(C)C)C2)ccc1N(C)c1cc2c(cn1)ncn2C] | (3- (dimethylamino)pyrrolidin- 1-yl)(3-ethyl-4- (methyl(1-methyl-1H- imidazo[4,5-c]pyridin-6- yl)amino)phenyl)methanone | H | 407 | 407 |
| 107 | 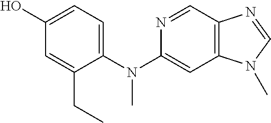 [SMILES: CCc1cc(O)ccc1N(C)c1cc2c(cn1)ncn2C] | 3-ethyl-4-(methyl(1- methyl-1H-imidazo[4,5- c]pyridin-6- yl)amino)phenol | Described above | 282 | 283 |
| 108 | 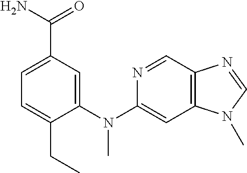 [SMILES: CCc1ccc(C(N)=O)cc1N(C)c1cc2c(cn1)ncn2C] | 4-ethyl-3-(methyl(1- methyl-1H-imidazo[4,5- c]pyridin-6- yl)amino)benzamide | M | 309 | 310 |
| 109 | 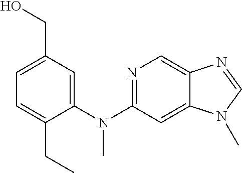 [SMILES: CCc1ccc(CO)cc1N(C)c1cc2c(cn1)ncn2C] | (4-ethyl-3-(methyl(1- methyl-1H-imidazo[4,5- c]pyridin-6- yl)amino)phenyl)MeOH | Described above | 296 | 297 |
| 110 | 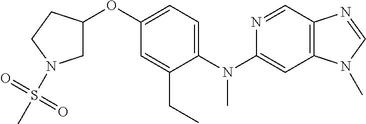 [SMILES: CCc1cc(OC2CCN(S(C)(=O)=O)C2)ccc1N(C)c1cc2c(cn1)ncn2C] | N-(2-ethyl-4-(1- (methylsulfonyl)pyrrolidin- 3-yloxy)phenyl)-N,1- dimethyl-1H-imidazo[4,5- c]pyridin-6-amine | Described above | 430 | 430 |
| 111 | 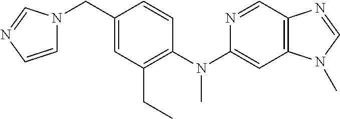 [SMILES: CCc1cc(Cn2ccnc2)ccc1N(C)c1cc2c(cn1)ncn2C] | N-(4-((1H-imidazol-1- yl)methyl)-2- ethylphenyl)-N,1- dimethyl-1H-imidazo[4,5- c]pyridin-6-amine | O | 346 | 347 |
| 112 | 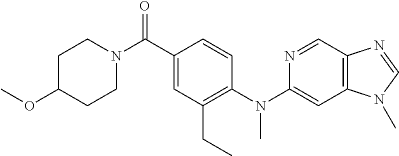 [SMILES: CCc1cc(C(=O)N2CCC(OC)CC2)ccc1N(C)c1cc2c(cn1)ncn2C] | (3-ethyl-4-(methyl(1- methyl-1H-imidazo[4,5- c]pyridin-6- yl)amino)phenyl)(4- methoxypiperidin-1- yl)methanone | Y | 408 | 408 |
| 113 | 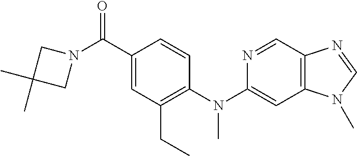 [SMILES: CCc1cc(C(=O)N2CC(C)(C)C2)ccc1N(C)c1cc2c(cn1)ncn2C] | (3,3-dimethylazetidin-1- yl)(3-ethyl-4-(methyl(1- methyl-1H-imidazo[4,5- c]pyridin-6- yl)amino)phenyl)methanone | Y | 377 | 378 |
| 114 | 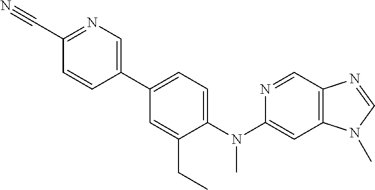 [SMILES: CCc1cc(-c2ccc(C#N)nc2)ccc1N(C)c1cc2c(cn1)ncn2C] | 5-(3-ethyl-4-(methyl(1- methyl-1H-imidazo[4,5- c]pyridin-6- yl)amino)phenyl)picolino- nitrile | F | 368 | 369 |
| 115 | 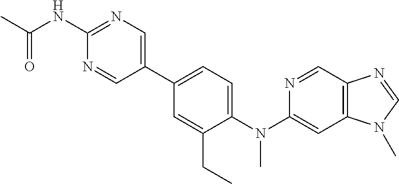 [SMILES: CCc1cc(-c2cnc(NC(C)=O)nc2)ccc1N(C)c1cc2c(cn1)ncn2C] | N-(5-(3-ethyl-4- (methyl(1-methyl-1H- imidazo[4,5-c]pyridin-6- yl)amino)phenyl)pyrimidin- 2-yl)acetamide | Described above | 401 | 402 |
| 116 | 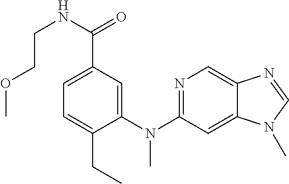 [SMILES: CCc1ccc(C(=O)NCCOC)cc1N(C)c1cc2c(cn1)ncn2C] | 4-ethyl-N-(2- methoxyethyl)-3- (methyl(1-methyl-1H- imidazo[4,5-c]pyridin-6- yl)amino)benzamide | Described above | 367 | 368 |
| 117 | 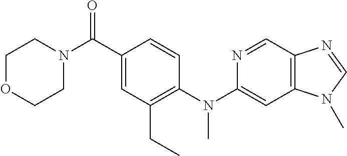 [SMILES: CCc1cc(C(=O)N2CCOCC2)ccc1N(C)c1cc2c(cn1)ncn2C] | (3-ethyl-4-(methyl(1- methyl-1H-imidazo[4,5- c]pyridin-6- yl)amino)phenyl)(morpho- lino)methanone | Y | 379 | 380 |
| 118 | 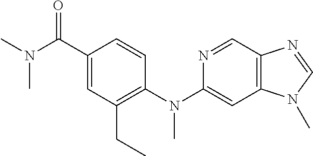 [SMILES: CCc1cc(C(=O)N(C)C)ccc1N(C)c1cc2c(cn1)ncn2C] | 3-ethyl-N,N-dimethyl-4- (methyl(1-methyl-1H- imidazo[4,5-c]pyridin-6- yl)amino)benzamide | Y | 337 | 338 |
| 119 | 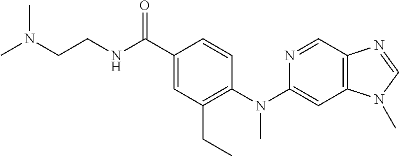 [SMILES: CCc1cc(C(=O)NCCN(C)C)ccc1N(C)c1cc2c(cn1)ncn2C] | N-(2- (dimethylamino)ethyl)-3- ethyl-4-(methyl(1-methyl- 1H-imidazo[4,5- c]pyridin-6- yl)amino)benzamide | Y | 380 | 381 |
| 120 | 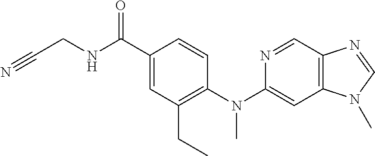 [SMILES: CCc1cc(C(=O)NCC#N)ccc1N(C)c1cc2c(cn1)ncn2C] | N-(cyanomethyl)-3-ethyl- 4-(methyl(1-methyl-1H- imidazo[4,5-c]pyridin-6- yl)amino)benzamide | Y | 348 | 349 |
| 121 | 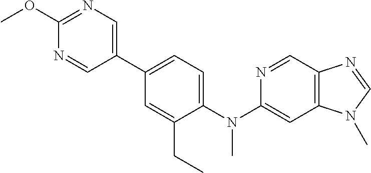 [SMILES: CCc1cc(-c2cnc(OC)nc2)ccc1N(C)c1cc2c(cn1)ncn2C] | N-(2-ethyl-4-(2- methoxypyrimidin-5- yl)phenyl)-N,1-dimethyl- 1H-imidazo[4,5- c]pyridin-6-amine | F | 374 | 375 |
| 122 | 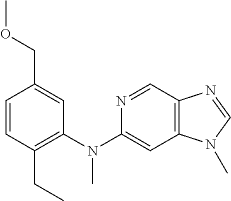 [SMILES: CCc1ccc(COC)cc1N(C)c1cc2c(cn1)ncn2C] | N-(2-ethyl-5- (methoxymethyl)phenyl)- N,1-dimethyl-1H- imidazo[4,5-c]pyridin-6- amine | Described above | 310 | 311 |
| 123 | 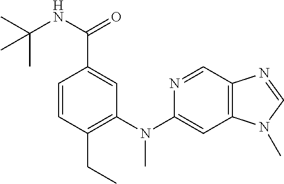 [SMILES: CCc1ccc(C(=O)NC(C)(C)C)cc1N(C)c1cc2c(cn1)ncn2C] | N-tert-butyl-4-ethyl-3- (methyl(1-methyl-1H- imidazo[4,5-c]pyridin-6- yl)amino)benzamide | Y | 365 | 366 |
| 124 | 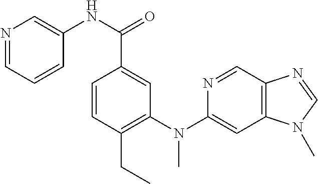 [SMILES: CCc1ccc(C(=O)Nc2cccnc2)cc1N(C)c1cc2c(cn1)ncn2C] | 4-ethyl-3-(methyl(1- methyl-1H-imidazo[4,5- c]pyridin-6-yl)amino)-N- (pyridin-3-yl)benzamide | Y | 386 | 387 |
| 125 | 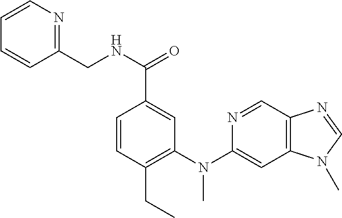 [SMILES: CCc1ccc(C(=O)NCc2ccccn2)cc1N(C)c1cc2c(cn1)ncn2C] | 4-ethyl-3-(methyl(1- methyl-1H-imidazo[4,5- c]pyridin-6-yl)amino)-N- (pyridin-2- ylmethyl)benzamide | Y | 400 | 401 |
| 126 | 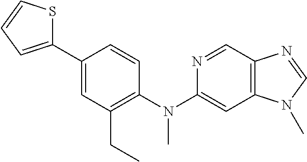 [SMILES: CCc1cc(-c2cccs2)ccc1N(C)c1cc2c(cn1)ncn2C] | N-(2-ethyl-4-(thiophen-2- yl)phenyl)-N,1-dimethyl- 1H-imidazo[4,5- c]pyridin-6-amine | F | 348 | 349 |
| 127 | 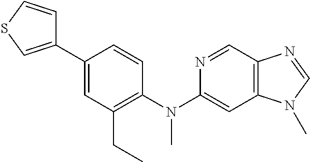 [SMILES: CCc1cc(-c2ccsc2)ccc1N(C)c1cc2c(cn1)ncn2C] | N-(2-ethyl-4-(thiophen-3- yl)phenyl)-N,1-dimethyl- 1H-imidazo[4,5- c]pyridin-6-amine | F | 348 | 349 |
| 128 | 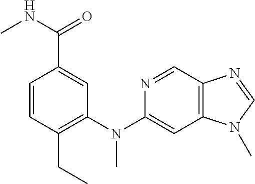 [SMILES: CCc1ccc(C(=O)NC)cc1N(C)c1cc2c(cn1)ncn2C] | 4-ethyl-N-methyl-3- (methyl(1-methyl-1H- imidazo[4,5-c]pyridin-6- yl)amino)benzamide | Y | 323 | 324 |
| 129 | 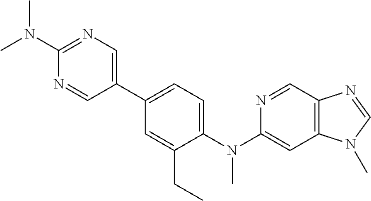 [SMILES: CCc1cc(-c2cnc(N(C)C)nc2)ccc1N(C)c1cc2c(cn1)ncn2C] | N-(4-(2- (dimethylamino)pyrimidin- 5-yl)-2-ethylphenyl)- N,1-dimethyl-1H- imidazo[4,5-c]pyridin-6- amine | N | 387 | 388 |
| 130 | 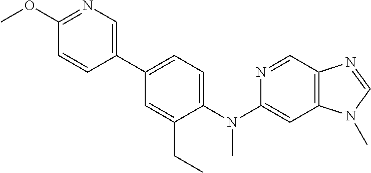 [SMILES: CCc1cc(-c2ccc(OC)nc2)ccc1N(C)c1cc2c(cn1)ncn2C] | N-(2-ethyl-4-(6- methoxypyridin-3- yl)phenyl)-N,1-dimethyl- 1H-imidazo[4,5- c]pyridin-6-amine | F | 373 | 374 |
| 131 | 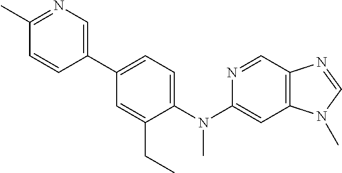 [SMILES: CCc1cc(-c2ccc(C)nc2)ccc1N(C)c1cc2c(cn1)ncn2C] | N-(2-ethyl-4-(6- methylpyridin-3- yl)phenyl)-N,1-dimethyl- 1H-imidazo[4,5- c]pyridin-6-amine | F | 357 | 358 |
| 132 | 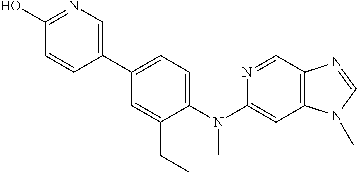 [SMILES: CCc1cc(-c2ccc(O)nc2)ccc1N(C)c1cc2c(cn1)ncn2C] | 5-(3-ethyl-4-(methyl(1- methyl-1H-imidazo[4,5- c]pyridin-6- yl)amino)phenyl)pyridin- 2-ol | Described above | 359 | 360 |
| 133 | 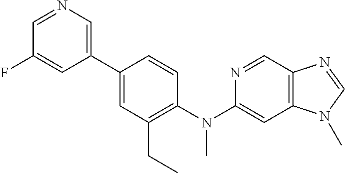 [SMILES: CCc1cc(-c2cncc(F)c2)ccc1N(C)c1cc2c(cn1)ncn2C] | N-(2-ethyl-4-(5- fluoropyridin-3- yl)phenyl)-N,1-dimethyl- 1H-imidazo[4,5- c]pyridin-6-amine | F | 361 | 362 |
| 134 | 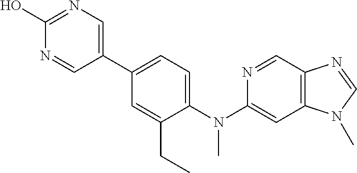 [SMILES: CCc1cc(-c2cnc(O)nc2)ccc1N(C)c1cc2c(cn1)ncn2C] | 5-(3-ethyl-4-(methyl(1- methyl-1H-imidazo[4,5- c]pyridin-6- yl)amino)phenyl)pyrimidin- 2-ol | Described above | 360 | 361 |
| 135 | 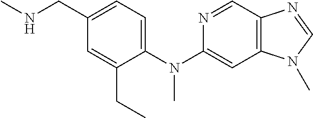 [SMILES: CCc1cc(CNC)ccc1N(C)c1cc2c(cn1)ncn2C] | N-(2-ethyl-4- ((methylamino)methyl) phenyl)-N,1-dimethyl-1H- imidazo[4,5-c]pyridin-6- amine | J | 309 | 310 |
| 136 | 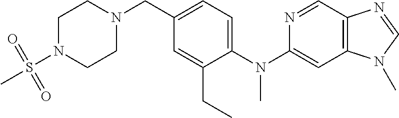 [SMILES: CCc1cc(CN2CCN(S(C)(=O)=O)CC2)ccc1N(C)c1cc2c(cn1)ncn2C] | N-(2-ethyl-4-((4- (methylsulfonyl)piperazin- 1-yl)methyl)phenyl)-N,1- dimethyl-1H-imidazo[4,5- c]pyridin-6-amine | Described above | 443 | 443 |
| 137 | 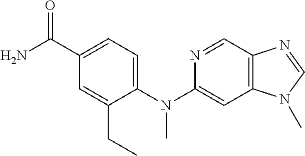 [SMILES: CCc1cc(C(N)=O)ccc1N(C)c1cc2c(cn1)ncn2C] | 3-ethyl-4-(methyl(1- methy-1H-imidazo[4,5- c]pyridin-6- yl)amino)benzamide | Described above | 309 | 310 |
| 138 | 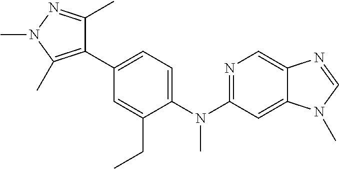 [SMILES: CCc1cc(-c2c(C)nn(C)c2C)ccc1N(C)c1cc2c(cn1)ncn2C] | N-(2-ethyl-4-(1,3,5- trimethyl-1H-pyrazol-4- yl)phenyl)-N,1-dimethyl- 1H-imidazo[4,5- c]pyridin-6-amine | F | 374 | 375 |
| 139 | 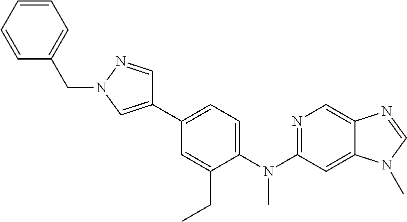 [SMILES: CCc1cc(-c2cnn(Cc3ccccc3)c2)ccc1N(C)c1cc2c(cn1)ncn2C] | N-(4-(1-benzyl-1H- pyrazol-4-yl)-2- ethylphenyl)-N,1- dimethyl-1H-imidazo[4,5- c]pyridin-6-amine | F | 423 | 423 |
| 140 | 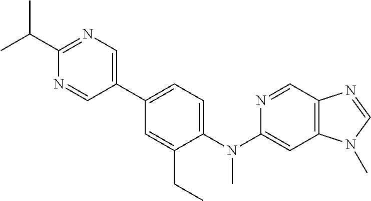 [SMILES: CCc1cc(-c2cnc(C(C)C)nc2)ccc1N(C)c1cc2c(cn1)ncn2C] | N-(2-ethyl-4-(2- isopropylpyrimidin-5- yl)phenyl)-N,1-dimethyl- 1H-imidazo[4,5- c]pyridin-6-amine | F | 387 | 387 |
| 141 | 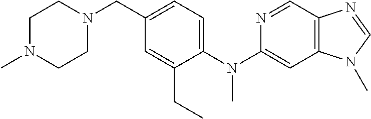 [SMILES: CCc1cc(CN2CCN(C)CC2)ccc1N(C)c1cc2c(cn1)ncn2C] | N-(2-ethyl-4-((4- methylpiperazin-1- yl)methyl)phenyl)-N,1- dimethyl-1H-imidazo[4,5- c]pyridin-6-amine | J | 379 | 379 |
| 142 | 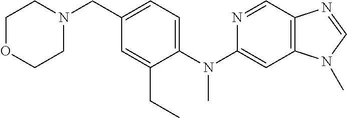 [SMILES: CCc1cc(CN2CCOCC2)ccc1N(C)c1cc2c(cn1)ncn2C] | N-(2-ethyl-4- (morpholinomethyl)phenyl)- N,1-dimethyl-1H- imidazo[4,5-c]pyridin-6- amine | J | 365 | 366 |
| 143 | 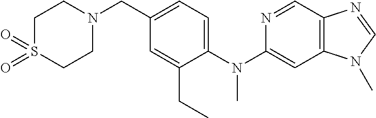 [SMILES: CCc1cc(CN2CCS(=O)(=O)CC2)ccc1N(C)c1cc2c(cn1)ncn2C] | [4-(1,1- Dioxothiomorpholin-4- ylmethyl)-2-ethyl- phenyl]-methyl-(1- methyl-1H-imidazo[4,5- c]pyridin-6-yl)-amine | J | 414 | 414 |
| 144 | 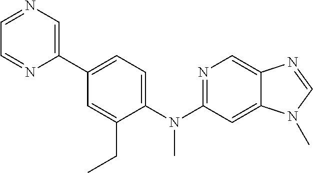 [SMILES: CCc1cc(-c2cnccn2)ccc1N(C)c1cc2c(cn1)ncn2C] | N-(2-ethyl-4-(pyrazin-2- yl)phenyl)-N,1-dimethyl- 1H-imidazo[4,5- c]pyridin-6-amine | F | 344 | 345 |
| 145 | 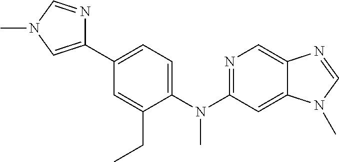 [SMILES: CCc1cc(-c2cn(C)cn2)ccc1N(C)c1cc2c(cn1)ncn2C] | N-(2-ethyl-4-(1-methyl- 1H-imidazol-4- yl)phenyl)-N,1-dimethyl- 1H-imidazo[4,5- c]pyridin-6-amine | F | 346 | 347 |
| 146 | 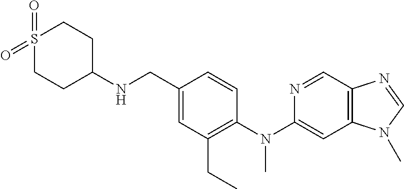 [SMILES: CCc1cc(CNC2CCS(=O)(=O)CC2)ccc1N(C)c1cc2c(cn1)ncn2C] | {4-[(1,1-Dioxo- hexahydrothiopyran-4- ylamino)-methyl]-2-ethyl- phenyl}-methyl-(1- methyl-1H-imidazo[4,5- c]pyridin-6-yl)-amine | J | 428 | 428 |
| 147 | 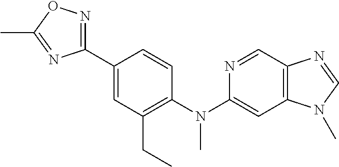 [SMILES: CCc1cc(-c2noc(C)n2)ccc1N(C)c1cc2c(cn1)ncn2C] | N-(2-ethyl-4-(5-methyl- 1,2,4-oxadiazol-3- yl)phenyl)-N,1-dimethyl- 1H-imidazo[4,5- c]pyridin-6-amine | F | 348 | 349 |
| 148 | 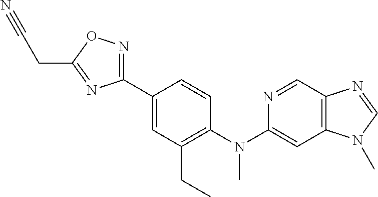 [SMILES: CCc1cc(-c2noc(CC#N)n2)ccc1N(C)c1cc2c(cn1)ncn2C] | 2-(3-(3-ethyl-4-(methyl(1- methyl-1H-imidazo[4,5- c]pyridin-6- yl)amino)phenyl)-1,2,4- oxadiazol-5- yl)acetonitrile | L | 373 | 374 |
| 149 | 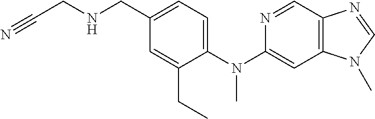 [SMILES: CCc1cc(CNCC#N)ccc1N(C)c1cc2c(cn1)ncn2C] | 2-(3-ethyl-4-(methyl(1- methyl-1H-imidazo[4,5- c]pyridin-6- yl)amino)benzylamino) acetonitrile | J | 334 | 335 |
| 150 | 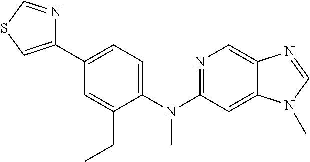 [SMILES: CCc1cc(-c2cscn2)ccc1N(C)c1cc2c(cn1)ncn2C] | N-(2-ethyl-4-(thiazol-4- yl)phenyl)-N,1-dimethyl- 1H-imidazo[4,5- c]pyridin-6-amine | L | 349 | 350 |
| 151 | 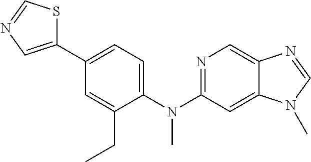 [SMILES: CCc1cc(-c2cncs2)ccc1N(C)c1cc2c(cn1)ncn2C] | N-(2-ethyl-4-(thiazol-5- yl)phenyl)-N,1-dimethyl- 1H-imidazo[4,5- c]pyridin-6-amine | Described above | 349 | 350 |
| 152 | 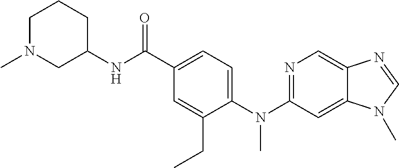 [SMILES: CCc1cc(C(=O)NC2CCCN(C)C2)ccc1N(C)c1cc2c(cn1)ncn2C] | 3-ethyl-4-(methyl(1- methyl-1H-imidazo[4,5- c]pyridin-6-yl)amino)-N- (1-methylpiperidin-3- yl)benzamide | Y | 407 | 407 |
| 153 | 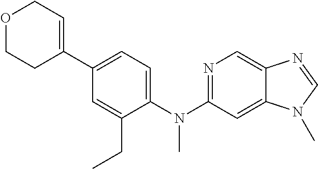 [SMILES: CCc1cc(C2=CCOCC2)ccc1N(C)c1cc2c(cn1)ncn2C] | N-(4-(3,6-dihydro-2H- pyran-4-yl)-2- ethylphenyl)-N,1- dimethyl-1H-imidazo[4,5- c]pyridin-6-amine | F | 348 | 349 |
| 154 | 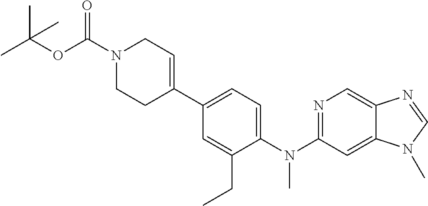 [SMILES: CCc1cc(C2=CCN(C(=O)OC(C)(C)C)CC2)ccc1N(C)c1cc2c(cn1)ncn2C] | tert-butyl 4-(3-ethyl-4- (methyl(1-methyl-1H- imidazo[4,5-c]pyridin-6- yl)amino)phenyl)-5,6- dihydropyridine-1(2H)- carboxylate | F | 448 | 448 |
| 155 | 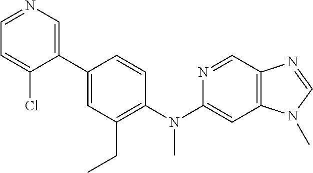 [SMILES: CCc1cc(-c2cnccc2Cl)ccc1N(C)c1cc2c(cn1)ncn2C] | N-(4-(4-chloropyridin-3- yl)-2-ethylphenyl)-N,1- dimethyl-1H-imidazo[4,5- c]pyridin-6-amine | F | 378 | 378 |
| 156 | 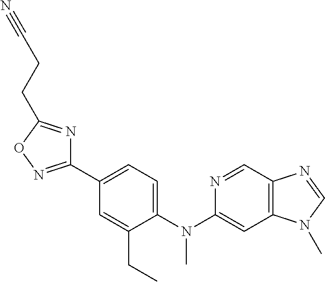 [SMILES: CCc1cc(-c2noc(CCC#N)n2)ccc1N(C)c1cc2c(cn1)ncn2C] | 3-(3-(3-ethyl-4-(methyl(1- methyl-1H-imidazo[4,5- c]pyridin-6- yl)amino)phenyl)-1,2,4- oxadiazol-5- yl)propanenitrile | described above | 387 | 388 |
| 157 | 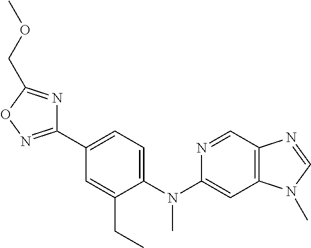 [SMILES: CCc1cc(-c2noc(COC)n2)ccc1N(C)c1cc2c(cn1)ncn2C] | N-(2-ethyl-4-(5- (methoxymethyl)-1,2,4- oxadiazol-3-yl)phenyl)- N,1-dimethyl-1H- imidazo[4,5-c]pyridin-6- amine | L | 378 | 379 |
| 158 | 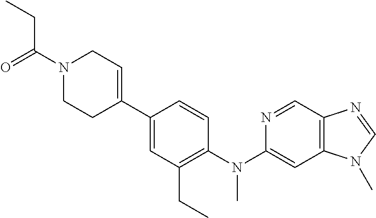 [SMILES: CCC(=O)N1CC=C(c2ccc(N(C)c3cc4c(cn3)ncn4C)c(CC)c2)CC1] | 1-(4-(3-ethyl-4-(methyl(1- methyl-1H-imidazo[4,5- c]pyridin-6- yl)amino)phenyl)-5,6- dihydropyridin-1(2H)- yl)propan-1-one | K | 404 | 404 |
| 159 | 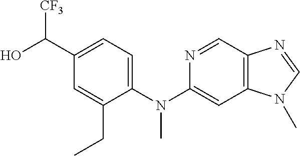 [SMILES: CCc1cc(C(O)C(F)(F)F)ccc1N(C)c1cc2c(cn1)ncn2C] | 1-(3-ethyl-4-(methyl(1- methyl-1H-imidazo[4,5- c]pyridin-6- yl)amino)phenyl)-2,2,2- trifluoroethanol | Described above | 364 | 365 |
| 160 | 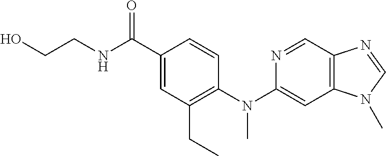 [SMILES: CCc1cc(C(=O)NCCO)ccc1N(C)c1cc2c(cn1)ncn2C] | 3-ethyl-N-(2- hydroxyethyl)-4- (methyl(1-methyl-1H- imidazo[4,5-c]pyridin-6- yl)amino)benzamide | Y | 353 | 354 |
| 161 | 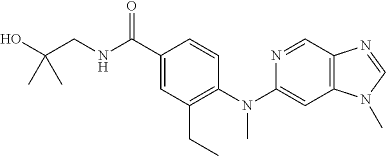 [SMILES: CCc1cc(C(=O)NCC(C)(C)O)ccc1N(C)c1cc2c(cn1)ncn2C] | 3-ethyl-N-(2-hydroxy-2- methylpropyl)-4- (methyl(1-methyl-1H- imidazo[4,5-c]pyridin-6- yl)amino)benzamide | Y | 381 | 382 |
| 162 | 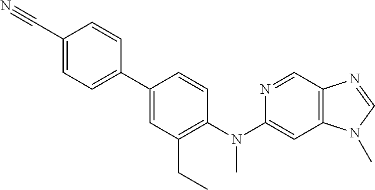 [SMILES: CCc1cc(-c2ccc(C#N)cc2)ccc1N(C)c1cc2c(cn1)ncn2C] | 3′-ethyl-4′-(methyl(1- methyl-1H-imidazo[4,5- c]pyridin-6- yl)amino)biphenyl-4- carbonitrile | Method F | 367 | 368 |
| 163 | 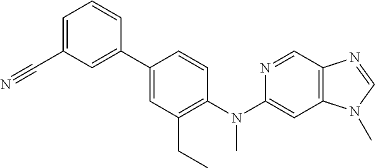 [SMILES: CCc1cc(-c2cccc(C#N)c2)ccc1N(C)c1cc2c(cn1)ncn2C] | 3′-ethyl-4′-(methyl(1- methyl-1H-imidazo[4,5- c]pyridin-6- yl)amino)biphenyl-3- carbonitrile | F | 367 | 368 |
| 164 | 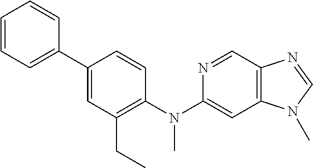 [SMILES: CCc1cc(-c2ccccc2)ccc1N(C)c1cc2c(cn1)ncn2C] | N-(3-ethylbiphenyl-4-yl)- N,1-dimethyl-1H- imidazo[4,5-c]pyridin-6- amine | F | 342 | 343 |
| 165 | 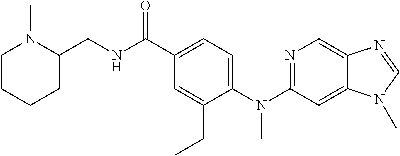 [SMILES: CCc1cc(C(=O)NCC2CCCCN2C)ccc1N(C)c1cc2c(cn1)ncn2C] | 3-ethyl-4-(methyl(1- methyl-1H-imidazo[4,5- c]pyridin-6-yl)amino)-N- ((1-methylpiperidin-2- yl)methyl)benzamide | Y | 421 | 421 |
| 166 | 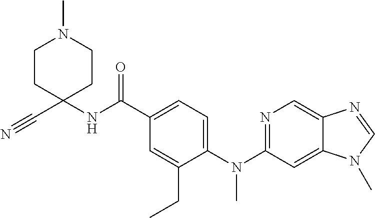 [SMILES: CCc1cc(C(=O)NC2(C#N)CCN(C)CC2)ccc1N(C)c1cc2c(cn1)ncn2C] | N-(4-cyano-1- methylpiperidin-4-yl)-3- ethyl-4-(methyl(1-methyl- 1H-imidazo[4,5- c]pyridin-6- yl)amino)benzamide | Y | 432 | 432 |
| 167 | 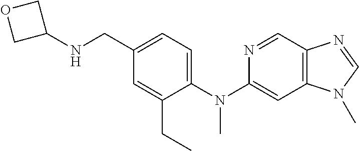 [SMILES: CCc1cc(CNC2COC2)ccc1N(C)c1cc2c(cn1)ncn2C] | N-(2-ethyl-4-((oxetan-3- ylamino)methyl)phenyl)- N,1-dimethyl-1H- imidazo[4,5-c]pyridin-6- amine | J | 351 | 352 |
| 168 | 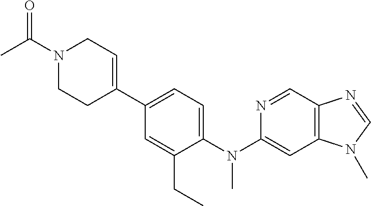 [SMILES: CCc1cc(C2=CCN(C(C)=O)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)-5,6- dihydropyridin-1(2H)- yl)ethanone | K | 390 | 390 |
| 169 | 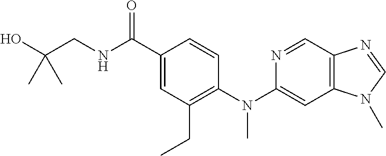 [SMILES: CCc1cc(C(=O)NCC(C)(C)O)ccc1N(C)c1cc2c(cn1)ncn2C] | 1-(3-ethyl-4-(methyl(1- methyl-1H-imidazo[4,5- c]pyridin-6- yl)amino)benzylamino)-2- methylpropan-2-ol | J | 367 | 368 |
| 170 | 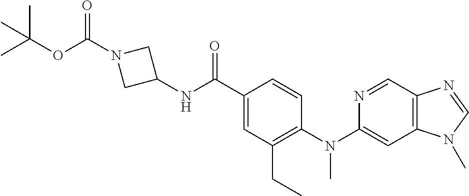 [SMILES: CCc1cc(C(=O)NC2CN(C(=O)OC(C)(C)C)C2)ccc1N(C)c1cc2c(cn1)ncn2C] | tert-butyl 3-(3-ethyl-4- (methyl(1-methyl-1H- imidazo[4,5-c]pyridin-6- yl)amino)benzamido)azetidine- 1-carboxylate | Described above | 465 | 465 |
| 171 | 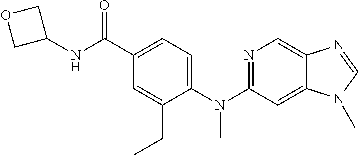 [SMILES: CCc1cc(C(=O)NC2COC2)ccc1N(C)c1cc2c(cn1)ncn2C] | 3-ethyl-4-(methyl(1- methyl-1H-imidazo[4,5- c]pyridin-6-yl)amino)-N- (oxetan-3-yl)benzamide | Y | 365 | 366 |
| 172 | 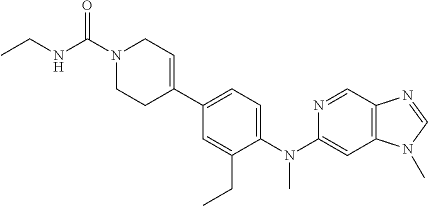 [SMILES: CCNC(=O)N1CC=C(c2ccc(N(C)c3cc4c(cn3)ncn4C)c(CC)c2)CC1] | N-ethyl-4-(3-ethyl-4- (methyl(1-methyl-1H- imidazo[4,5-c]pyridin-6- yl)amino)phenyl)-5,6- dihydropyridine-1(2H)- carboxamide | V | 419 | 419 |
| 173 | 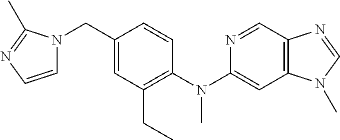 [SMILES: CCc1cc(Cn2ccnc2C)ccc1N(C)c1cc2c(cn1)ncn2C] | N-(2-ethyl-4-((2-methyl- 1H-imidazol-1- yl)methyl)phenyl)-N,1- dimethyl-1H-imidazo[4,5- c]pyridin-6-amine | Described above | 360 | 361 |
| 174 | 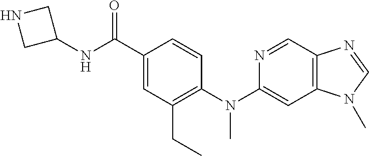 [SMILES: CCc1cc(C(=O)NC2CNC2)ccc1N(C)c1cc2c(cn1)ncn2C] | N-(azetidin-3-yl)-3-ethyl- 4-(methyl(1-methyl-1H- imidazo[4,5-c]pyridin-6- yl)amino)benzamide | Described above | 364 | 365 |
| 175 | 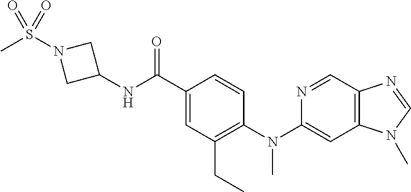 [SMILES: CCc1cc(C(=O)NC2CN(S(C)(=O)=O)C2)ccc1N(C)c1cc2c(cn1)ncn2C] | 3-ethyl-4-(methyl(1- methyl-1H-imidazo[4,5- c]pyridin-6-yl)amino)-N- (1- (methylsulfonyl)azetidin- 3-yl)benzamide | Described above | 443 | 443 |
| 176 | 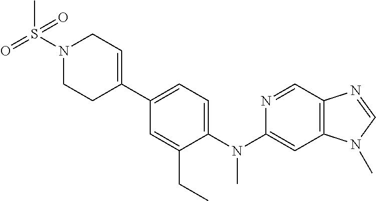 [SMILES: CCc1cc(C2=CCN(S(C)(=O)=O)CC2)ccc1N(C)c1cc2c(cn1)ncn2C] | N-(2-ethyl-4-(1- (methylsulfonyl)-1,2,3,6- tetrahydropyridin-4- yl)phenyl)-N,1-dimethyl- 1H-imidazo[4,5- c]pyridin-6-amine | S | 426 | 426 |
| 177 | 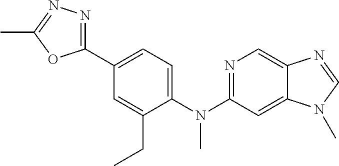 [SMILES: CCc1cc(-c2nnc(C)o2)ccc1N(C)c1cc2c(cn1)ncn2C] | N-(2-ethyl-4-(5-methyl- 1,3,4-oxadiazol-2- yl)phenyl)-N,1-dimethyl- 1H-imidazo[4,5- c]pyridin-6-amine | Described above | 348 | 349 |
| 178 | 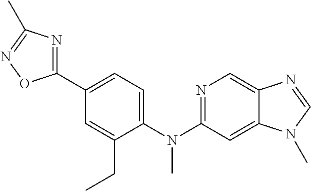 [SMILES: CCc1cc(-c2nc(C)no2)ccc1N(C)c1cc2c(cn1)ncn2C] | N-(2-ethyl-4-(3-methyl- 1,2,4-oxadiazol-5- yl)phenyl)-N,1-dimethyl- 1H-imidazo[4,5- c]pyridin-6-amine | Described above | 348 | 349 |
| 179 | 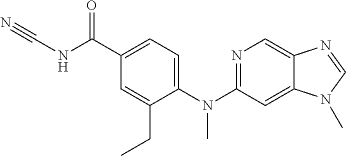 [SMILES: CCc1cc(C(=O)NC#N)ccc1N(C)c1cc2c(cn1)ncn2C] | N-cyano-3-ethyl-4- (methyl(1-methyl-1H- imidazo[4,5-c]pyridin-6- yl)amino)benzamide | Described above | 334 | 335 |
| 180 | 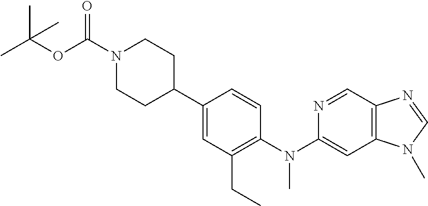 [SMILES: CCc1cc(C2CCN(C(=O)OC(C)(C)C)CC2)ccc1N(C)c1cc2c(cn1)ncn2C] | tert-butyl 4-(3-ethyl-4- (methyl(1-methyl-1H- imidazo[4,5-c]pyridin-6- yl)amino)phenyl)piperidine- 1-carboxylate | Described above to make 187 | 450 | 450 |
| 181 | 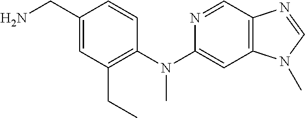 [SMILES: CCc1cc(CN)ccc1N(C)c1cc2c(cn1)ncn2C] | N-(4-(aminomethyl)-2- ethylphenyl)-N,1- dimethyl-1H-imidazo[4,5- c]pyridin-6-amine | Described above | 295 | 296 |
| 182 | 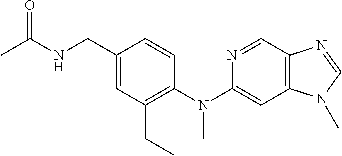 [SMILES: CCc1cc(CNC(C)=O)ccc1N(C)c1cc2c(cn1)ncn2C] | N-(3-ethyl-4-(methyl(1- methyl-1H-imidazo[4,5- c]pyridin-6- yl)amino)benzyl)acetamide | Described above | 337 | 338 |
| 183 | 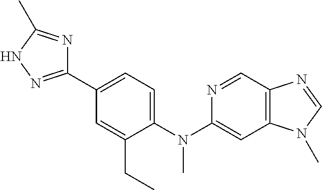 [SMILES: CCc1cc(-c2n[nH]c(C)n2)ccc1N(C)c1cc2c(cn1)ncn2C] | N-(2-ethyl-4-(5-methyl- 1H-1,2,4-triazol-3- yl)phenyl)-N,1-dimethyl- 1H-imidazo[4,5- c]pyridin-6-amine | R | 347 | 348 |
| 184 | 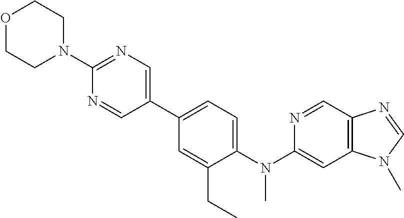 [SMILES: CCc1cc(-c2cnc(N3CCOCC3)nc2)ccc1N(C)c1cc2c(cn1)ncn2C] | N-(2-ethyl-4-(2- morpholinopyrimidin-5- yl)phenyl)-N,1-dimethyl- 1H-imidazo[4,5- c]pyridin-6-amine | Described above | 430 | 430 |
| 185 | 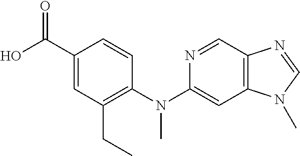 [SMILES: CCc1cc(C(=O)O)ccc1N(C)c1cc2c(cn1)ncn2C] | 3-ethyl-4-(methyl(1- methyl-1H-imidazo[4,5- c]pyridin-6- yl)amino)benzic acid | Described above | 310 | 311 |
| 186 | 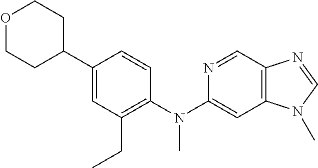 [SMILES: CCc1cc(C2CCOCC2)ccc1N(C)c1cc2c(cn1)ncn2C] | N-(2-ethyl-4-(tetrahydro- 2H-pyran-4-yl)phenyl)- N,1-dimethyl-1H- imidazo[4,5-c]pyridin-6- amine | Described above | 350 | 351 |
| 187 | 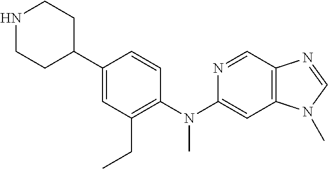 [SMILES: CCc1cc(C2CCNCC2)ccc1N(C)c1cc2c(cn1)ncn2C] | N-(2-ethyl-4-(piperidin-4- yl)phenyl)-N,1-dimethyl- 1H-imidazo[4,5- c]pyridin-6-amine | Described above | 349 | 350 |
| 188 | 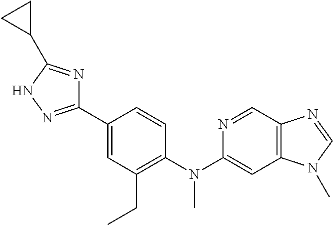 [SMILES: CCc1cc(-c2n[nH]c(C3CC3)n2)ccc1N(C)c1cc2c(cn1)ncn2C] | N-(4-(5-cyclopropyl-1H- 1,2,4-triazol-3-yl)-2- ethylphenyl)-N,1- dimethyl-1H-imidazo[4,5- c]pyridin-6-amine | R | 373 | 374 |
| 189 | 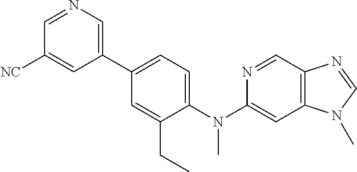 [SMILES: CCc1cc(-c2cncc(C#N)c2)ccc1N(C)c1cc2c(cn1)ncn2C] | 5-(3-ethyl-4-(methyl(1- methyl-1H-imidazo[4,5- c]pyridin-6- yl)amino)phenyl)nicotino- nitrile | F | 368 | 369 |
| 190 | 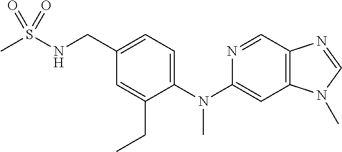 [SMILES: CCc1cc(CNS(C)(=O)=O)ccc1N(C)c1cc2c(cn1)ncn2C] | N-(3-ethyl-4-(methyl(1- methyl-1H-imidazo[4,5- c]pyridin-6- yl)amino)benzyl)methane sulfonamide | Described above | 373 | 374 |
| 191 | 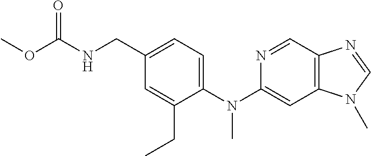 [SMILES: CCc1cc(CNC(=O)OC)ccc1N(C)c1cc2c(cn1)ncn2C] | methyl 3-ethyl-4- (methyl(1-methyl-1H- imidazo[4,5-c]pyridin-6- yl)amino)benzylcarbamate | Described above | 353 | 354 |
| 192 | 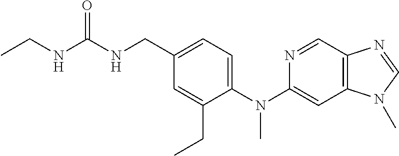 [SMILES: CCNC(=O)NCc1ccc(N(C)c2cc3c(cn2)ncn3C)c(CC)c1] | 1-ethyl-3-(3-ethyl-4- (methyl(1-methyl-1H- imidazo[4,5-c]pyridin-6- yl)amino)benzyl)urea | Described above | 366 | 367 |
| 193 | 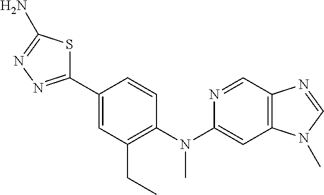 [SMILES: CCc1cc(-c2nnc(N)s2)ccc1N(C)c1cc2c(cn1)ncn2C] | 5-(3-ethyl-4-(methyl(1- methyl-1H-imidazo[4,5- c]pyridin-6- yl)amino)phenyl)-1,3,4- thiadiazol-2-amine | Described above | 365 | 366 |
| 194 | 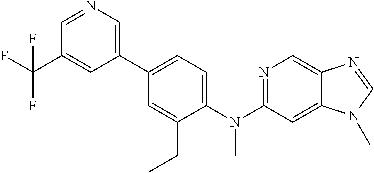 [SMILES: CCc1cc(-c2cncc(C(F)(F)F)c2)ccc1N(C)c1cc2c(cn1)ncn2C] | N-(2-ethyl-4-(5- (trifluoromethyl)pyridin- 3-yl)phenyl)-N,1- dimethyl-1H-imidazo[4,5- c]pyridin-6-amine | F | 411 | 412 |
| 195 | 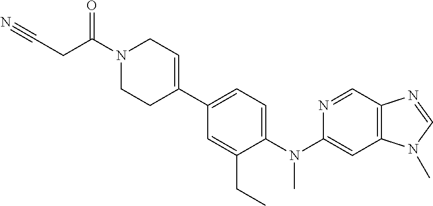 [SMILES: CCc1cc(C2=CCN(C(=O)CC#N)CC2)ccc1N(C)c1cc2c(cn1)ncn2C] | 3-(4-(3-ethyl-4-(methyl(1- methyl-1H-imidazo[4,5- c]pyridin-6- yl)amino)phenyl)-5,6- dihydropyridin-1(2H)-yl)- 3-oxopropanenitrile | Described above | 415 | 415 |
| 196 | 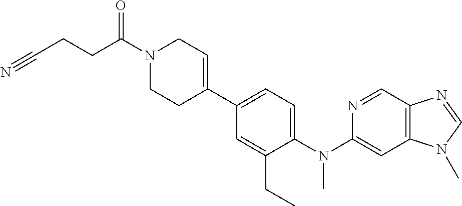 [SMILES: CCc1cc(C2=CCN(C(=O)CCC#N)CC2)ccc1N(C)c1cc2c(cn1)ncn2C] | 4-(4-(3-ethyl-4-(methyl(1- methyl-1H-imidazo[4,5- c]pyridin-6- yl)amino)phenyl)-5,6- dihydropyridin-1(2H)-yl)- 4-oxobutanenitrile | K | 429 | 429 |
| 197 | 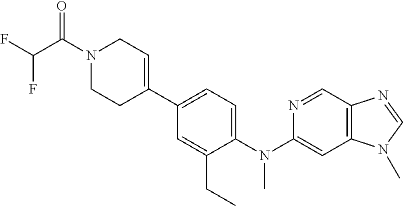 [SMILES: CCc1cc(C2=CCN(C(=O)C(F)F)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)-5,6- dihydropyridin-1(2H)-yl)- 2,2-difluoroethanone | K | 425 | 426 |
| 198 | 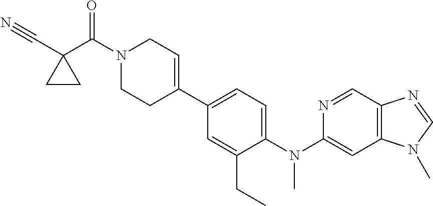 [SMILES: CCc1cc(C2=CCN(C(=O)C3(C#N)CC3)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)-1,2,3,6- tetrahydropyridine-1- carbonyl)cyclopropane- carbonitrile | K | 441 | 441 |
| 199 | 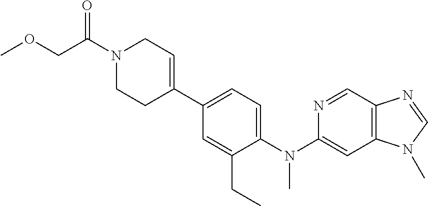 [SMILES: CCc1cc(C2=CCN(C(=O)COC)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)-5,6- dihydropyridin-1(2H)-yl)- 2-methoxyethanone | K | 420 | 420 |
| 200 | 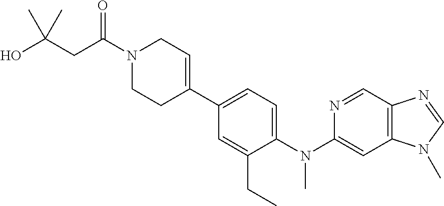 [SMILES: CCc1cc(C2=CCN(C(=O)CC(C)(C)O)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)-5,6- dihydropyridin-1(2H)-yl)- 3-hydroxy-3- methylbutan-1-one | K | 448 | 448 |
| 201 | 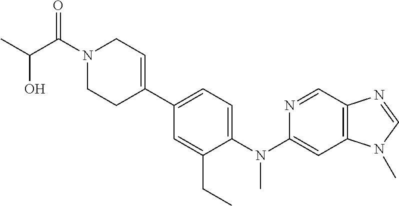 [SMILES: CCc1cc(C2=CCN(C(=O)C(C)O)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)-5,6- dihydropyridin-1(2H)-yl)- 2-hydroxypropan-1-one | K | 420 | 420 |
| 202 | 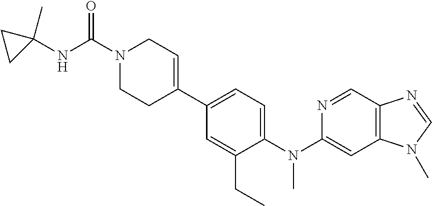 [SMILES: CCc1cc(C2=CCN(C(=O)NC3(C)CC3)CC2)ccc1N(C)c1cc2c(cn1)ncn2C] | 4-(3-ethyl-4-(methyl(1- methyl-1H-imidazo[4,5- c]pyridin-6- yl)amino)phenyl)-N-(1- methylcyclopropyl)-5,6- dihydropyridine-1(2H)- carboxamide | S | 445 | 445 |
| 203 | 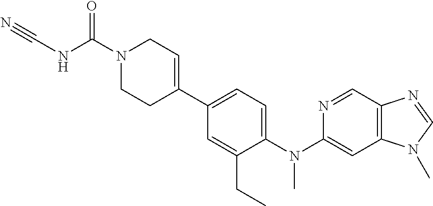 [SMILES: CCc1cc(C2=CCN(C(=O)NC#N)CC2)ccc1N(C)c1cc2c(cn1)ncn2C] | N-cyano-4-(3-ethyl-4- (methyl(1-methyl-1H- imidazo[4,5-c]pyridin-6- yl)amino)phenyl)-5,6- dihydropyridine-1(2H)- carboxamide | S | 416 | 416 |
| 204 | 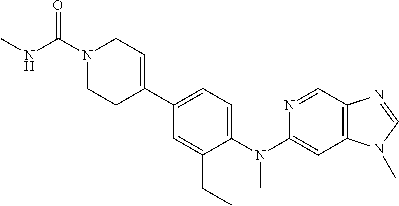 [SMILES: CCc1cc(C2=CCN(C(=O)NC)CC2)ccc1N(C)c1cc2c(cn1)ncn2C] | 4-(3-ethyl-4-(methyl(1- methyl-1H-imidazo[4,5- c]pyridin-6- yl)amino)phenyl)-N- methyl-5,6- dihydropyridine-1(2H)- carboxamide | V | 405 | 405 |
| 205 | 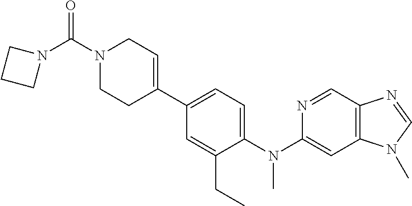 [SMILES: CCc1cc(C2=CCN(C(=O)N3CCC3)CC2)ccc1N(C)c1cc2c(cn1)ncn2C] | azetidin-1-yl(4-(3-ethyl-4- (methyl(1-methyl-1H- imidazo[4,5-c]pyridin-6- yl)amino)phenyl)-5,6- dihydropyridin-1(2H)- yl)methanone | S | 431 | 431 |
| 206 | 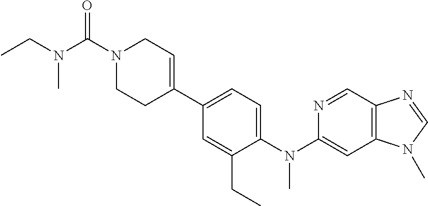 [SMILES: CCc1cc(C2=CCN(C(=O)N(C)CC)CC2)ccc1N(C)c1cc2c(cn1)ncn2C] | N-ethyl-4-(3-ethyl-4- (methyl(1-methyl-1H- imidazo[4,5-c]pyridin-6- yl)amino)phenyl)-N- methyl-5,6- dihydropyridine-1(2H)- carboxamide | S | 433 | 433 |
| 207 | 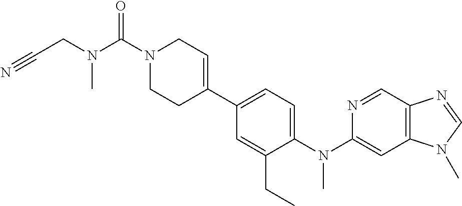 [SMILES: CCc1cc(C2=CCN(C(=O)N(C)CC#N)CC2)ccc1N(C)c1cc2c(cn1)ncn2C] | N-(cyanomethyl)-4-(3- ethyl-4-(methyl(1-methyl- 1H-imidazo[4,5- c]pyridin-6- yl)amino)phenyl)-N- methyl-5,6- dihydropyridine-1(2H)- carboxamide | Described above | 444 | 444 |
| 208 | 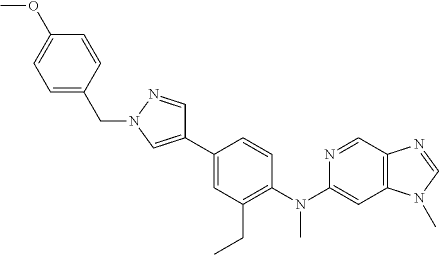 [SMILES: CCc1cc(-c2cnn(Cc3ccc(OC)cc3)c2)ccc1N(C)c1cc2c(cn1)ncn2C] | N-(2-ethyl-4-(1-(4- methoxybenzyl)-1H- pyrazol-4-yl)phenyl)-N,1- dimethyl-1H-imidazo[4,5- c]pyridin-6-amine | X | 453 | 423 |
| 209 | 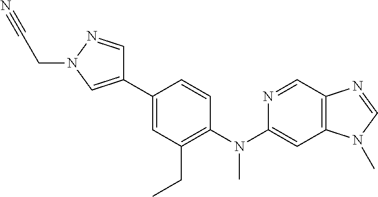 [SMILES: CCc1cc(-c2cnn(CC#N)c2)ccc1N(C)c1cc2c(cn1)ncn2C] | 2-(4-(3-ethyl-4-(methyl(1- methyl-1H-imidazo[4,5- c]pyridin-6- yl)amino)phenyl)-1H- pyrazol-1-yl)acetonitrile | X | 371 | 372 |
| 210 | 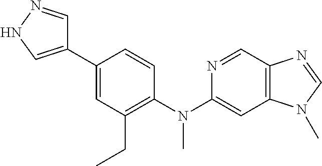 [SMILES: CCc1cc(-c2cn[nH]c2)ccc1N(C)c1cc2c(cn1)ncn2C] | N-(2-ethyl-4-(1H-pyrazol- 4-yl)phenyl)-N,1- dimethyl-1H-imidazo[4,5- c]pyridin-6-amine | Described above in method X | 332 | 333 |
| 211 | 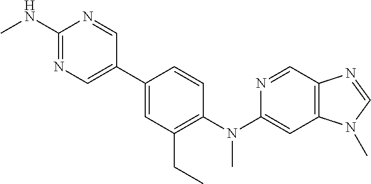 [SMILES: CCc1cc(-c2cnc(NC)nc2)ccc1N(C)c1cc2c(cn1)ncn2C] | N-(2-ethyl-4-(2- (methylamino)pyrimidin- 5-yl)phenyl)-N,1- dimethyl-1H-imidazo[4,5- c]pyridin-6-amine | Described above | 373 | 374 |
| 212 | 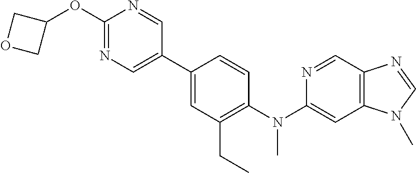 [SMILES: CCc1cc(-c2cnc(OC3COC3)nc2)ccc1N(C)c1cc2c(cn1)ncn2C] | N-(2-ethyl-4-(2-(oxetan- 3-yloxy)pyrimidin-5- yl)phenyl)-N,1-dimethyl- 1H-imidazo[4,5- c]pyridin-6-amine | Described above | 416 | 417 |
| 213 | 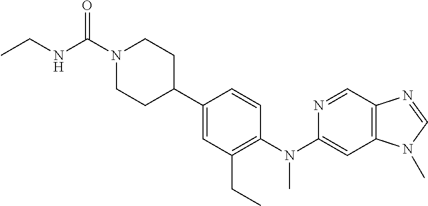 [SMILES: CCNC(=O)N1CCC(c2ccc(N(C)c3cc4c(cn3)ncn4C)c(CC)c2)CC1] | N-ethyl-4-(3-ethyl-4- (methyl(1-methyl-1H- imidazo[4,5-c]pyridin-6- yl)amino)phenyl)piperidine- 1-carboxamide | Described above | 421 | 421 |
| 214 | 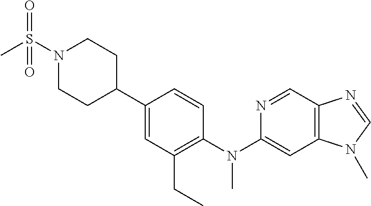 [SMILES: CCc1cc(C2CCN(S(C)(=O)=O)CC2)ccc1N(C)c1cc2c(cn1)ncn2C] | N-(2-ethyl-4-(1- (methylsulfonyl)piperidin- 4-yl)phenyl)-N,1- dimethyl-1H-imidazo[4,5- c]pyridin-6-amine | com- pound 187 + U | 428 | 428 |
| 215 | 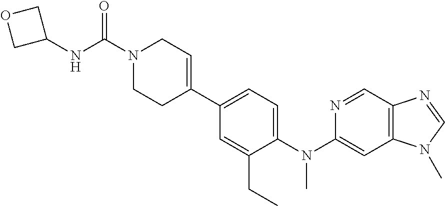 [SMILES: CCc1cc(C2=CCN(C(=O)NC3COC3)CC2)ccc1N(C)c1cc2c(cn1)ncn2C] | 4-(3-ethyl-4-(methyl(1- ethyl-1H-imidazo[4,5- c]pyridin-6- yl)amino)phenyl)-N- (oxetan-3-yl)-5,6- dihydropyridine-1(2H)- carboxamide | S | 447 | 447 |
| 216 | 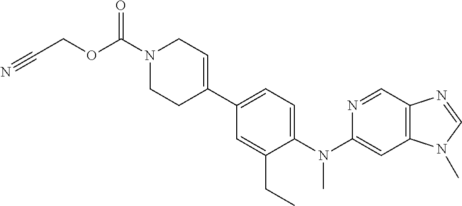 [SMILES: CCc1cc(C2=CCN(C(=O)OCC#N)CC2)ccc1N(C)c1cc2c(cn1)ncn2C] | cyanomethyl 4-(3-ethyl-4- (methyl(1-methyl-1H- imidazo[4,5-c]pyridin-6- yl)amino)phenyl)-5,6- dihydropyridine-1(2H)- carboxylate | Described above | 431 | 431 |
| 217 | 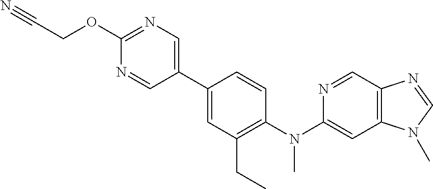 [SMILES: CCc1cc(-c2cnc(OCC#N)nc2)ccc1N(C)c1cc2c(cn1)ncn2C] | 2-(5-(3-ethyl-4-(methyl(1- methyl-1H-imidazo[4,5- c]pyridin-6- yl)amino)phenyl)pyrimidin- 2-yloxy)acetonitrile | W | 399 | 400 |
| 218 | 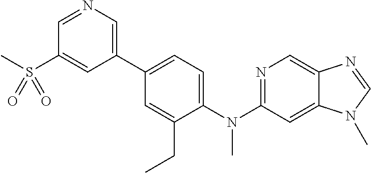 [SMILES: CCc1cc(-c2cncc(S(C)(=O)=O)c2)ccc1N(C)c1cc2c(cn1)ncn2C] | N-(2-ethyl-4-(5- (methylsulfonyl)pyridin- 3-yl)phenyl)-N,1- dimethyl-1H-imidazo[4,5- c]pyridin-6-amine | F | 422 | 422 |
| 219 | 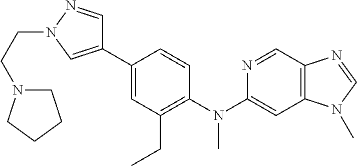 [SMILES: CCc1cc(-c2cnn(CCN3CCCC3)c2)ccc1N(C)c1cc2c(cn1)ncn2C] | N-(2-ethyl-4-(1-(2- (pyrrolidin-1-yl)ethyl)- 1H-pyrazol-4-yl)phenyl)- N,1-dimethyl-1H- imidazo[4,5-c]pyridin-6- amine | X | 430 | 430 |
| 220 | 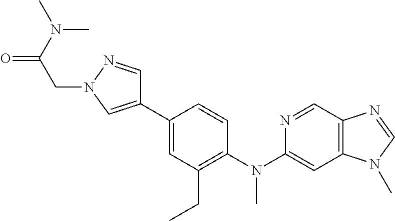 [SMILES: CCc1cc(-c2cnn(CC(=O)N(C)C)c2)ccc1N(C)c1cc2c(cn1)ncn2C] | 2-(4-(3-ethyl-4-(methyl(1- methyl-1H-imidazo[4,5- c]pyridin-6- yl)amino)phenyl)-1H- pyrazol-1-yl)-N,N- dimethylacetamide | X | 418 | 418 |
| 221 | 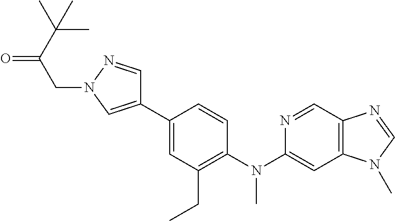 [SMILES: CCc1cc(-c2cnn(CC(=O)C(C)(C)C)c2)ccc1N(C)c1cc2c(cn1)ncn2C] | 1-(4-(3-ethyl-4-(methyl(1- methyl-1H-imidazo[4,5- c]pyridin-6- yl)amino)phenyl)-1H- pyrazol-1-yl)-3,3- dimethylbutan-2-one | X | 431 | 431 |
| 222 | 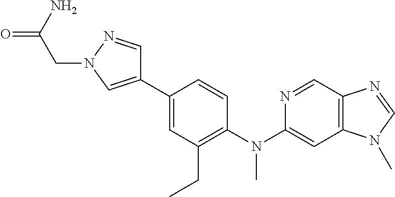 [SMILES: CCc1cc(-c2cnn(CC(N)=O)c2)ccc1N(C)c1cc2c(cn1)ncn2C] | 2-(4-(3-ethyl-4-(methyl(1- methyl-1H-imidazo[4,5- c]pyridin-6- yl)amino)phenyl)-1H- pyrazol-1-yl)acetamide | Described above | 389 | 390 |
| 223 | 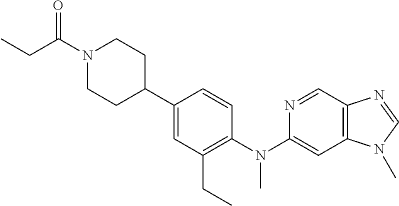 [SMILES: CCC(=O)N1CCC(c2ccc(N(C)c3cc4c(cn3)ncn4C)c(CC)c2)CC1] | 1-(4-(3-ethyl-4-(methyl(1- methyl-1H-imidazo[4,5- c]pyridin-6- yl)amino)phenyl)piperidin- 1-yl)propan-1-one | com- pound 187 and K | 406 | 406 |
| 224 | 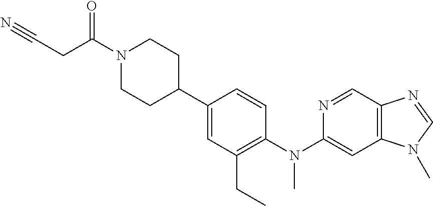 [SMILES: CCc1cc(C2CCN(C(=O)CC#N)CC2)ccc1N(C)c1cc2c(cn1)ncn2C] | 3-(4-(3-ethyl-4-(methyl(1- methyl-1H-imidazo[4,5- c]pyridin-6- yl)amino)phenyl)piperidin- 1-yl)-3-oxopropanenitrile | Com- pound 187 and K | 417 | 417 |
| 225 | 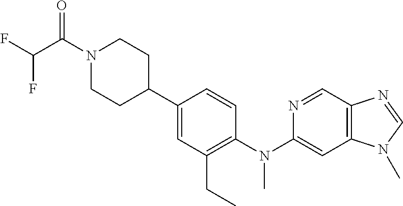 [SMILES: CCc1cc(C2CCN(C(=O)C(F)F)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)piperidin- 1-yl)-2,2- difluoroethanone | com- pound 187 and K | 427 | 428 |
| 226 | 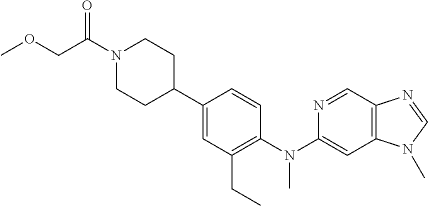 [SMILES: CCc1cc(C2CCN(C(=O)COC)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)piperidin- 1-yl)-2-methoxyethanone | com- pound 187 and K | 422 | 422 |
| 227 | 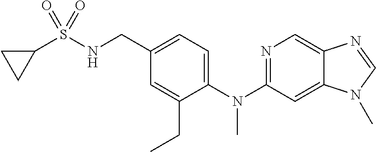 [SMILES: CCc1cc(CNS(=O)(=O)C2CC2)ccc1N(C)c1cc2c(cn1)ncn2C] | N-(3-ethyl-4-(methyl(1- methyl-1H-imidazo[4,5- c]pyridin-6- yl)amino)benzyl)cyclopro- panesulfonamide | T | 400 | 401 |
| 228 | 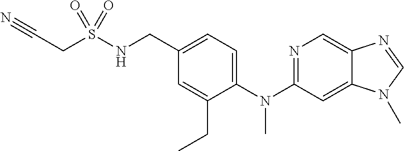 [SMILES: CCc1cc(CNS(=O)(=O)CC#N)ccc1N(C)c1cc2c(cn1)ncn2C] | 1-cyano-N-(3-ethyl-4- (methyl(1-methyl-1H- imidazo[4,5-c]pyridin-6- yl)amino)benzyl)methane- sulfonamide | T | 398 | 399 |
| 229 | 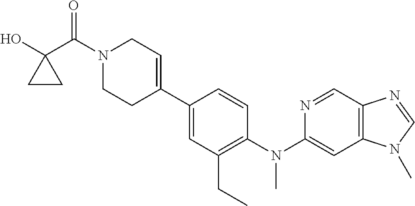 [SMILES: CCc1cc(C2=CCN(C(=O)C3(O)CC3)CC2)ccc1N(C)c1cc2c(cn1)ncn2C] | (4-(3-ethyl-4-(methyl(1- methyl-1H-imidazo[4,5- c]pyridin-6- yl)amino)phenyl)-5,6- dihydropyridin-1(2H)- yl)(1- hydroxycyclopropyl)meth- anone | K | 432 | 433 |
| 230 | 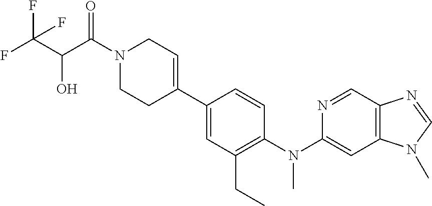 [SMILES: CCc1cc(C2=CCN(C(=O)C(O)C(F)(F)F)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)-5,6- dihydropyridin-1(2H)-yl)- 3,3,3-trifluoro-2- hydroxypropan-1-one | K | 473 | 474 |
| 231 | 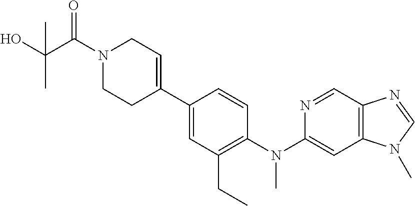 [SMILES: CCc1cc(C2=CCN(C(=O)C(C)(C)O)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)-5,6- dihydropyridin-1(2H)-yl)- 2-hydroxy-2- methylpropan-1-one | K | 434 | 435 |
| 232 | 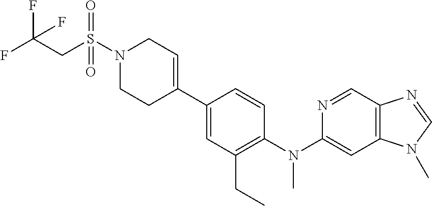 [SMILES: CCc1cc(C2=CCN(S(=O)(=O)CC(F)(F)F)CC2)ccc1N(C)c1cc2c(cn1)ncn2C] | N-(2-ethyl-4-(1-(2,2,2- trifluoroethylsulfonyl)- 1,2,3,6-tetrahydropyridin- 4-yl)phenyl)-N,1- dimethyl-1H-imidazo[4,5- c]pyridin-6-amine | U | 494 | 495 |
| 233 | 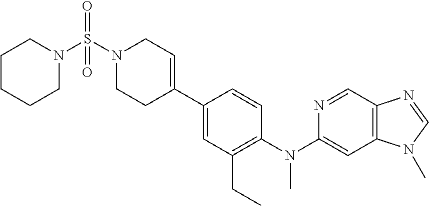 [SMILES: CCc1cc(C2=CCN(S(=O)(=O)N3CCCCC3)CC2)ccc1N(C)c1cc2c(cn1)ncn2C] | N-(2-ethyl-4-(1- (piperidin-1-ylsulfonyl)- 1,2,3,6-tetrahydropyridin- 4-yl)phenyl)-N,1- dimethyl-1H-imidazo[4,5- c]pyridin-6-amine | U | 495 | 496 |
| 234 | 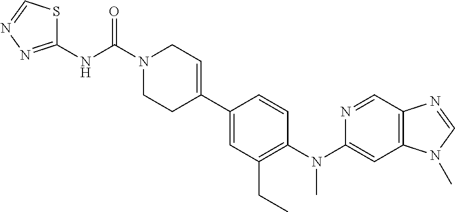 [SMILES: CCc1cc(C2=CCN(C(=O)Nc3nncs3)CC2)ccc1N(C)c1cc2c(cn1)ncn2C] | 4-(3-ethyl-4-(methyl(1- methyl-1H-imidazo[4,5- c]pyridin-6- yl)amino)phenyl)-N- (1,3,4-thiadiazol-2-yl)- 5,6-dihydropyridine- 1(2H)-carboxamide | Described above | 475 | 476 |
| 235 | 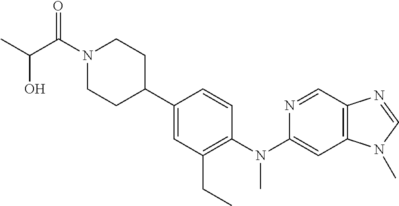 [SMILES: CCc1cc(C2CCN(C(=O)C(C)O)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)piperidin- 1-yl)-2-hydroxypropan- 1-one | K | 422 | 423 |
| 236 | 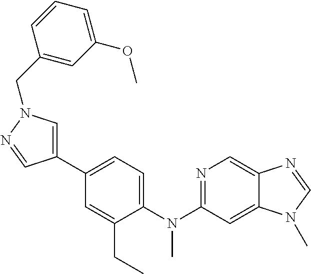 [SMILES: CCc1cc(-c2cnn(Cc3cccc(OC)c3)c2)ccc1N(C)c1cc2c(cn1)ncn2C] | N-(2-ethyl-4-(1-(3- methoxybenzyl)-1H- pyrazol-4-yl)phenyl)-N,1- dimethyl-1H-imidazo[4,5- c]pyridin-6-amine | X | 453 | 454 |
| 237 | 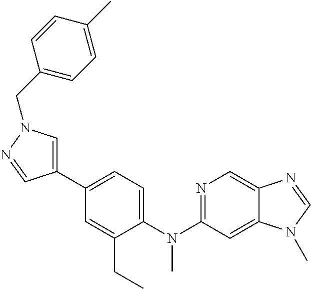 [SMILES: CCc1cc(-c2cnn(Cc3ccc(C)cc3)c2)ccc1N(C)c1cc2c(cn1)ncn2C] | N-(2-ethyl-4-(1-(4- methylbenzyl)-1H- pyrazol-4-yl)phenyl)-N,1- dimethyl-1H-imidazo[4,5- c]pyridin-6-amine | X | 437 | 438 |
| 238 | 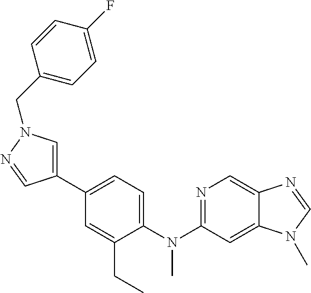 [SMILES: CCc1cc(-c2cnn(Cc3ccc(F)cc3)c2)ccc1N(C)c1cc2c(cn1)ncn2C] | N-(2-ethyl-4-(1-(4- fluorobenzyl)-1H- pyrazol-4-yl)phenyl)-N,1- dimethyl-1H-imidazo[4,5- c]pyridin-6-amine | X | 441 | 239 |
| 239 | 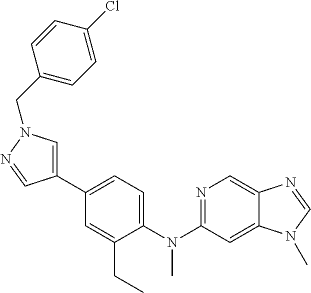 [SMILES: CCc1cc(-c2cnn(Cc3ccc(Cl)cc3)c2)ccc1N(C)c1cc2c(cn1)ncn2C] | N-(4-(1-(4-chlorobenzyl)- 1H-pyrazol-4-yl)-2- ethylphenyl)-N,1- dimethyl-1H-imidazo[4,5- c]pyridin-6-amine | X | 457 | 458 |
| 240 | 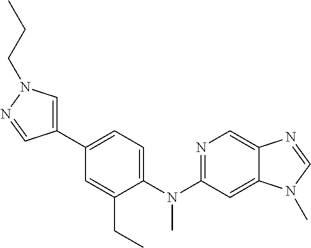 [SMILES: CCCn1cc(-c2ccc(N(C)c3cc4c(cn3)ncn4C)c(CC)c2)cn1] | N-(2-ethyl-4-(1-propyl- 1H-pyrazol-4-yl)phenyl)- N,1-dimethyl-1H- imidazo[4,5-c]pyridin-6- amine | X | 374 | 375 |
| 241 | 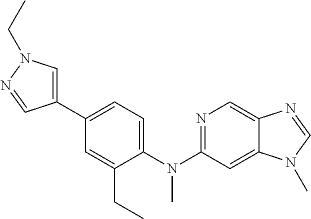 [SMILES: CCc1cc(-c2cnn(CC)c2)ccc1N(C)c1cc2c(cn1)ncn2C] | N-(2-ethyl-4-(1-ethyl-1H- pyrazol-4-yl)phenyl)-N,1- dimethyl-1H-imidazo[4,5- c]pyridin-6-amine | X | 360 | 361 |
| 242 | 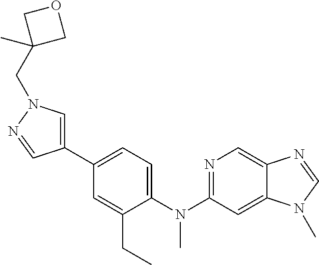 [SMILES: CCc1cc(-c2cnn(CC3(C)COC3)c2)ccc1N(C)c1cc2c(cn1)ncn2C] | N-(2-ethyl-4-(1-((3- methyloxetan-3- yl)methyl)-1H-pyrazol-4- yl)phenyl)-N,1-dimethyl- 1H-imidazo[4,5- c]pyridin-6-amine | X | 417 | 418 |
| 243 | 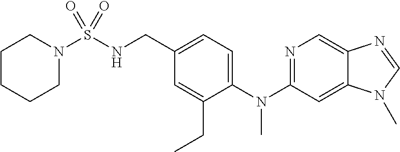 [SMILES: CCc1cc(CNS(=O)(=O)N2CCCCC2)ccc1N(C)c1cc2c(cn1)ncn2C] | N-(3-ethyl-4-(methyl(1- methyl-1H-imidazo[4,5- c]pyridin-6- yl)amino)benzyl)piperidine- 1-sulfonamide | T | 443 | 444 |
| 244 | 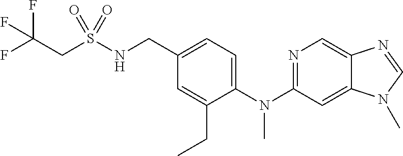 [SMILES: CCc1cc(CNS(=O)(=O)CC(F)(F)F)ccc1N(C)c1cc2c(cn1)ncn2C] | N-(3-ethyl-4-(methyl(1- methyl-1H-imidazo[4,5- c]pyridin-6- yl)amino)benzyl)-2,2,2- trifluoroethane sulfonamide | T | 441 | 442 |
| 245 | 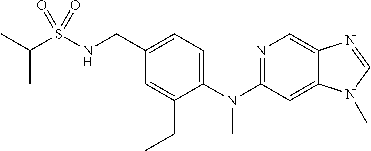 [SMILES: CCc1cc(CNS(=O)(=O)C(C)C)ccc1N(C)c1cc2c(cn1)ncn2C] | N-(3-ethyl-4-(methyl(1- methyl-1H-imidazo[4,5- c]pyridin-6- yl)amino)benzyl)propane- 2-sulfonamide | T | 402 | 403 |
| 246 | 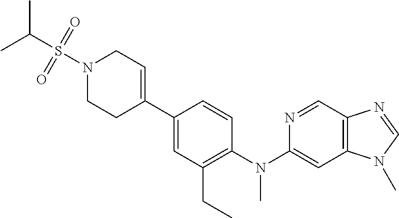 [SMILES: CCc1cc(C2=CCN(S(=O)(=O)C(C)C)CC2)ccc1N(C)c1cc2c(cn1)ncn2C] | N-(2-ethyl-4-(1- (isopropylsulfonyl)- 1,2,3,6-tetrahydropyridin- 4-yl)phenyl)-N,1- dimethyl-1H-imidazo[4,5- c]pyridin-6-amine | U | 454 | 455 |
| 247 | 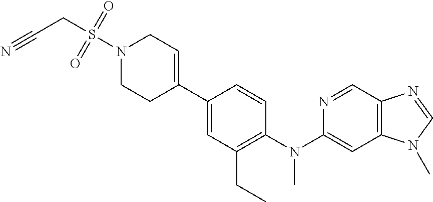 [SMILES: CCc1cc(C2=CCN(S(=O)(=O)CC#N)CC2)ccc1N(C)c1cc2c(cn1)ncn2C] | 2-(4-(3-ethyl-4-(methyl(1- methyl-1H-imidazo[4,5- c]pyridin-6- yl)amino)phenyl)-5,6- dihydropyridin-1(2H)- ylsulfonyl)acetonitrile | U | 451 | 452 |
| 248 | 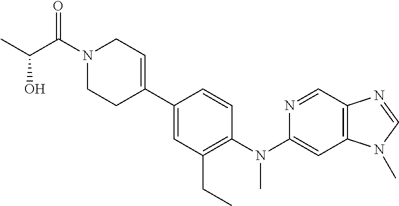 [SMILES: CCc1cc(C2=CCN(C(=O)[C@@H](C)O)CC2)ccc1N(C)c1cc2c(cn1)ncn2C] | (R)-1-(4-(3-ethyl-4- (methyl(1-methyl-1H- imidazo[4,5-c]pyridin-6- yl)amino)phenyl)-5,6- dihydropyridin-1(2H)-yl)- 2-hydroxypropan-1-one | K | 420 | 421 |
| 249 | 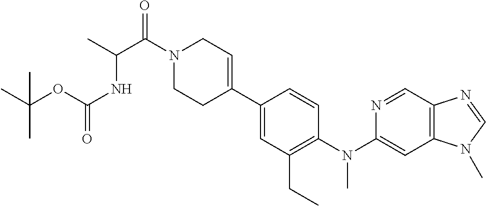 [SMILES: CCc1cc(C2=CCN(C(=O)C(C)NC(=O)OC(C)(C)C)CC2)ccc1N(C)c1cc2c(cn1)ncn2C] | tert-butyl 1-(4-(3-ethyl-4- (methyl(1-methyl-1H- imidazo[4,5-c]pyridin-6- yl)amino)phenyl)-5,6- dihydropyridin-1(2H)-yl)- 1-oxopropan-2- ylcarbamate | K | 519 | 520 |
| 250 | 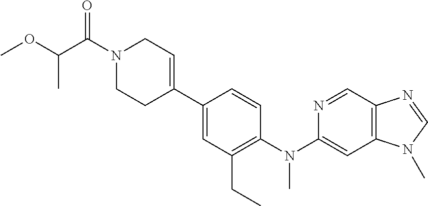 [SMILES: CCc1cc(C2=CCN(C(=O)C(C)OC)CC2)ccc1N(C)c1cc2c(cn1)ncn2C] | 1-(4-(3-ethyl-4-(methyl(1- methyl-1H-imidazo[4,5- c]pyridin-6- yl)amino)phenyl)-5,6- dihydropyridin-1(2H)-yl)- 2-methoxypropan-1-one | K | 434 | 435 |
| 251 | 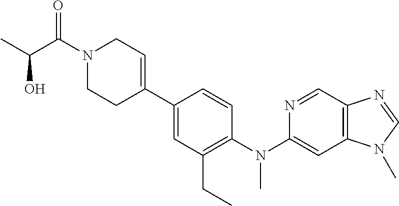 [SMILES: CCc1cc(C2=CCN(C(=O)[C@H](C)O)CC2)ccc1N(C)c1cc2c(cn1)ncn2C] | (S)-1-(4-(3-ethyl-4- (methyl(1-methyl-1H- imidazo[4,5-c]pyridin-6- yl)amino)phenyl)-5,6- dihydropyridin-1(2H)-yl)- 2-hydroxypropan-1-one | K | 420 | 421 |
| 252 | 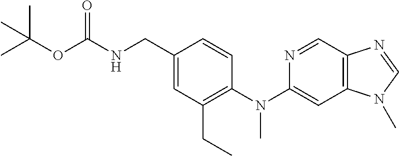 [SMILES: CCc1cc(CNC(=O)OC(C)(C)C)ccc1N(C)c1cc2c(cn1)ncn2C] | tert-butyl 3-ethyl-4- (methyl(1-methyl-1H- imidazo[4,5-c]pyridin-6- yl)amino)benzylcarbamate | Described above | 396 | 397 |
| 253 | 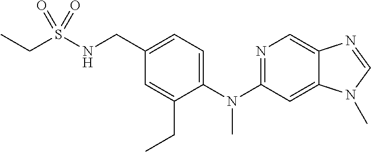 [SMILES: CCc1cc(CNS(=O)(=O)CC)ccc1N(C)c1cc2c(cn1)ncn2C] | N-(3-ethyl-4-(methyl(1- methyl-1H-imidazo[4,5- c]pyridin-6- yl)amino)benzyl)ethane- sulfonamide | T | 388 | 389 |
| 254 | 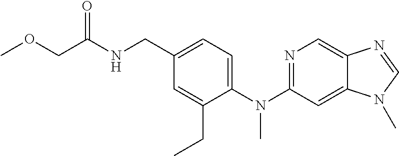 [SMILES: CCc1cc(CNC(=O)COC)ccc1N(C)c1cc2c(cn1)ncn2C] | N-(3-ethyl-4-(methyl(1- methyl-1H-imidazo[4,5- c]pyridin-6- yl)amino)benzyl)-2- methoxyacetamide | Described above | 367 | 368 |
| 255 | 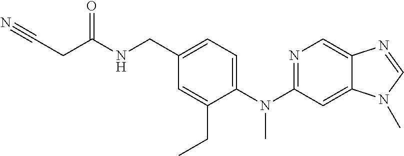 [SMILES: CCc1cc(CNC(=O)CC#N)ccc1N(C)c1cc2c(cn1)ncn2C] | 2-cyano-N-(3-ethyl-4- (methyl(1-methyl-1H- imidazo[4,5-c]pyridin-6- yl)amino)benzyl)acetamide | Same as 254 | 362 | 363 |
| 256 | 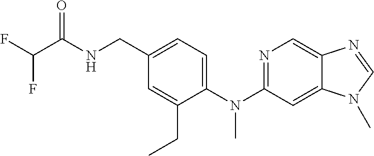 [SMILES: CCc1cc(CNC(=O)C(F)F)ccc1N(C)c1cc2c(cn1)ncn2C] | N-(3-ethyl-4-(methyl(1- methyl-1H-imidazo[4,5- c]pyridin-6- yl)amino)benzyl)-2,2- difluoroacetamide | Same as 254 | 373 | 374 |
| 257 | 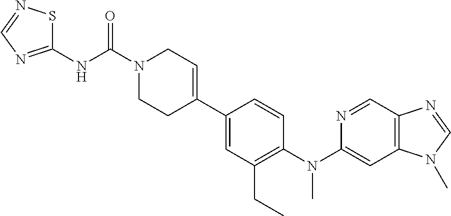 [SMILES: CCc1cc(C2=CCN(C(=O)Nc3ncns3)CC2)ccc1N(C)c1cc2c(cn1)ncn2C] | 4-(3-ethyl-4-(methyl(1- methyl-1H-imidazo[4,5- c]pyridin-6- yl)amino)phenyl)-N- (1,2,4-thiadiazol-5-yl)- 5,6-dihydropyridine- 1(2H)-carboxamide | Described above | 475 | 476 |
| 258 | 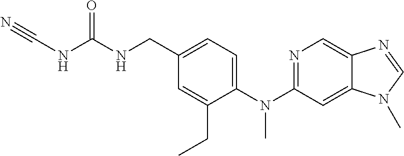 [SMILES: CCc1cc(CNC(=O)NC#N)ccc1N(C)c1cc2c(cn1)ncn2C] | 1-{3-Ethyl-4-[methyl-(1- methyl-1H-imidazo[4,5- c]pyridin-6-yl)-amino]- benzyl}-3-cyano-urea | Described above | 363 | 364 |
| 259 | 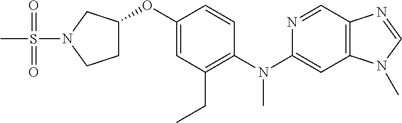 [SMILES: CCc1cc(O[C@@H]2CCN(S(C)(=O)=O)C2)ccc1N(C)c1cc2c(cn1)ncn2C] | (R)-N-(2-ethyl-4-(1- (methylsulfonyl)pyrrolidin- 3-yloxy)phenyl)-N,1- dimethyl-1H-imidazo[4,5- c]pyridin-6-amine | Same as 110 | 430 | 431 |
| 260 | 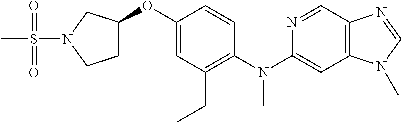 [SMILES: CCc1cc(O[C@H]2CCN(S(C)(=O)=O)C2)ccc1N(C)c1cc2c(cn1)ncn2C] | (S)-N-(2-ethyl-4-(1- (methylsulfonyl)pyrrolidin- 3-yloxy)phenyl)-N,1- dimethyl-1H-imidazo[4,5- c]pyridin-6-amine | Same as c 110 | 430 | 431 |
| 261 | 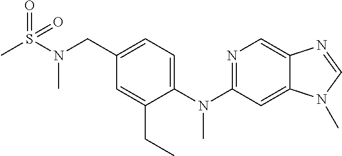 [SMILES: CCc1cc(CN(C)S(C)(=O)=O)ccc1N(C)c1cc2c(cn1)ncn2C] | N-(3-ethyl-4-(methyl(1- methyl-1H-imidazo[4,5- c]pyridin-6- yl)amino)benzyl)-N- methylmethanesulfonamide | Described above | 388 | 389 |
| 262 | 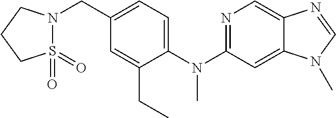 [SMILES: CCc1cc(CN2CCCS2(=O)=O)ccc1N(C)c1cc2c(cn1)ncn2C] | [4-(1,1- Dioxoisothiazolidin-2- ylmethyl)-2-ethyl- phenyl]-methyl-(1- methyl-1H-imidazo[4,5- c]pyridin-6-yl)-amine | Described above | 400 | 401 |
| 263 | 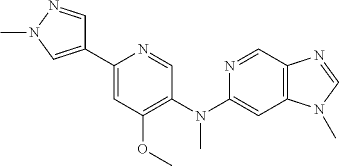 [SMILES: COc1cc(-c2cnn(C)c2)ncc1N(C)c1cc2c(cn1)ncn2C] | N-(4-methoxy-6-(1- methyl-1H-pyrazol-4- yl)pyridin-3-yl)-N,1- dimethyl-1H-imidazo[4,5- c]pyridin-6-amine | Same as 264 | 349 | 350 |
| 264 | 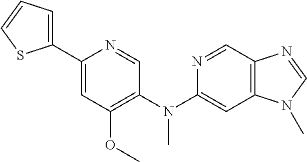 [SMILES: COc1cc(-c2cccs2)ncc1N(C)c1cc2c(cn1)ncn2C] | N-(4-methoxy-6- (thiophen-2-yl)pyridin-3- yl)-N,1-dimethyl-1H- imidazo[4,5-c]pyridin-6- amine | Described above | 351 | 352 |
| 265 | 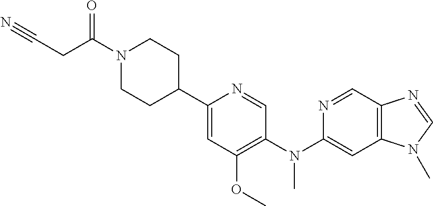 [SMILES: COc1cc(C2CCN(C(=O)CC#N)CC2)ncc1N(C)c1cc2c(cn1)ncn2C] | 3-(4-(4-methoxy-5- (methyl(1-methyl-1H- imidazo[4,5-c]pyridin-6- yl)amino)pyridin-2- yl)piperidin-1-yl)-3- oxopropanenitrile | Described above | 419 | 420 |
| 266 | 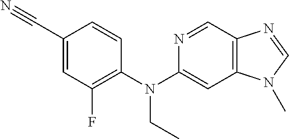 [SMILES: CCN(c1cc2c(cn1)ncn2C)c1ccc(C#N)cc1F] | 4-(ethyl(1-methyl-1H- imidazo[4,5-c]pyridin-6- yl)amino)-3- fluorobenzonitrile | A and E | 295 | 296 |
| 267 | 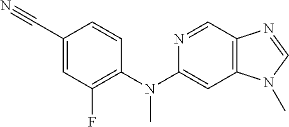 [SMILES: CN(c1cc2c(cn1)ncn2C)c1ccc(C#N)cc1F] | 3-fluoro-4-(methyl(1- methyl-1H-imidazo[4,5- c]pyridin-6- yl)amino)benzonitrile | A and B | 281 | 282 |
| 268 | 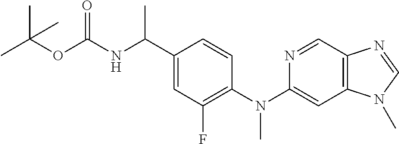 [SMILES: CC(NC(=O)OC(C)(C)C)c1ccc(N(C)c2cc3c(cn2)ncn3C)c(F)c1] | tert-butyl 1-(3-fluoro-4- (methyl(1-methyl-1H- imidazo[4,5-c]pyridin-6- yl)amino)phenyl)ethyl- carbamate | Described above | 399 | 400 |
| 269 | 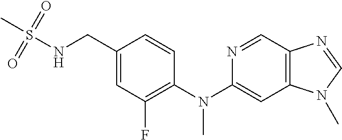 [SMILES: CN(c1cc2c(cn1)ncn2C)c1ccc(CNS(C)(=O)=O)cc1F] | N-(3-fluoro-4-(methyl(1- methyl-1H-imidazo[4,5- c]pyridin-6- yl)amino)benzyl)methane- sulfonamide | Described above | 363 | 364 |
| 270 | 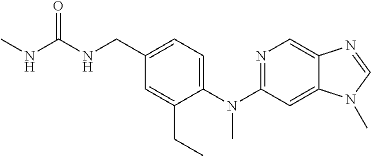 [SMILES: CCc1cc(CNC(=O)NC)ccc1N(C)c1cc2c(cn1)ncn2C] | 1-(3-ethyl-4-(methyl(1- methyl-1H-imidazo[4,5- c]pyridin-6- yl)amino)benzyl)-3- methylurea | Described above | 352 | 353 |
| 271 | 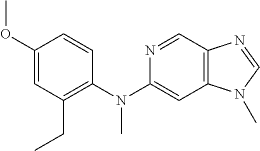 [SMILES: CCc1cc(OC)ccc1N(C)c1cc2c(cn1)ncn2C] | N-(2-ethyl-4- methoxyphenyl)-N,1- dimethyl-1H-imidazo[4,5- c]pyridin-6-amine | Described above | 296 | 297 |
| 272 | 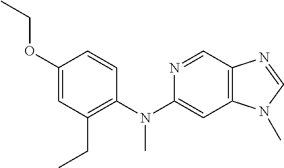 [SMILES: CCOc1ccc(N(C)c2cc3c(cn2)ncn3C)c(CC)c1] | N-(4-ethoxy-2- ethylphenyl)-N,1- dimethyl-1H-imidazo[4,5- c]pyridin-6-amine | Described above | 310 | 311 |
| 273 | 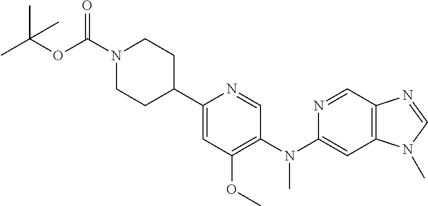 [SMILES: COc1cc(C2CCN(C(=O)OC(C)(C)C)CC2)ncc1N(C)c1cc2c(cn1)ncn2C] | tert-butyl 4-(4-methoxy- 5-(methyl(1-methyl-1H- imidazo[4,5-c]pyridin-6- yl)amino)pyridin-2- yl)piperidine-1- carboxylate | Described above | 453 | 454 |
| 274 | 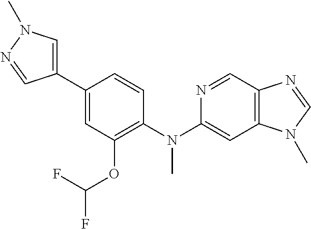 [SMILES: CN(c1cc2c(cn1)ncn2C)c1ccc(-c2cnn(C)c2)cc1OC(F)F] | N-(2-(difluoromethoxy)- 4-(1-methyl-1H-pyrazol- 4-yl)phenyl)-N,1- dimethyl-1H-imidazo[4,5- c]pyridin-6-amine | Described above | 384 | 385 |
| 275 | 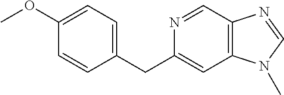 [SMILES: COc1ccc(Cc2cc3c(cn2)ncn3C)cc1] | 6-(4-methoxybenzyl)-1- methyl-1H-imidazo[4,5- c]pyridine | Described above | 253 | 254 |
| 276 | 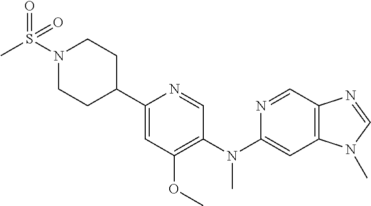 [SMILES: COc1cc(C2CCN(S(C)(=O)=O)CC2)ncc1N(C)c1cc2c(cn1)ncn2C] | (1′-Methanesulfonyl-4- methoxy-1′,2′,3′,4′,5′,6′- hexahydro- [2,4]bipyridinyl-5-yl)- methyl-(1-methyl-1H- imidazo[4,5-c]pyridin-6- yl)-amine | Described above | 430 | 431 |
| 277 | 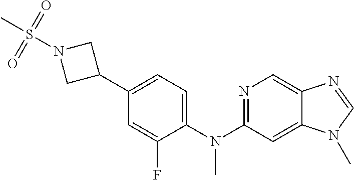 [SMILES: CN(c1cc2c(cn1)ncn2C)c1ccc(C2CN(S(C)(=O)=O)C2)cc1F] | [2-Fluoro-4-(1- methanesulfonyl-azetidin- 3-yl)-phenyl]-methyl-(1- methyl-1H-imidazo[4,5- c]pyridin-6-yl)-amine | Described above | 389 | 390 |
| 278 | 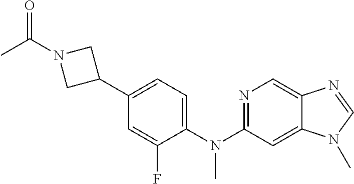 [SMILES: CC(=O)N1CC(c2ccc(N(C)c3cc4c(cn3)ncn4C)c(F)c2)C1] | 1-(3-{3-Fluoro-4-[methyl- (1-methyl-1H- imidazo[4,5-c]pyridin-6- yl)-amino]-phenyl}- azetidin-1-yl)-ethanone | Described above | 353 | 354 |
| 279 | 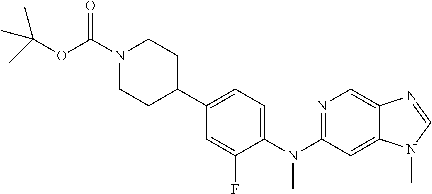 [SMILES: CN(c1cc2c(cn1)ncn2C)c1ccc(C2CCN(C(=O)OC(C)(C)C)CC2)cc1F] | 4-{3-Fluoro-4-[methyl-(1- methyl-1H-imidazo[4,5- c]pyridin-6-yl)-amino]- phenyl}-piperidine-1- carboxylic acid tert-butyl ester | Described above | 439 | 440 |
| 280 | 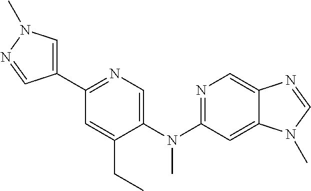 [SMILES: CCc1cc(-c2cnn(C)c2)ncc1N(C)c1cc2c(cn1)ncn2C] | [4-Ethyl-6-(1-methyl-1H- pyrazol-4-yl)-pyridin-3- yl]-methyl-(1-methyl-1H- imidazo[4,5-c]pyridin-6- yl)-amine | Described above | 347 | 348 |
| 281 | 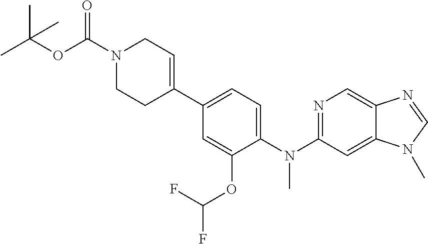 [SMILES: CN(c1cc2c(cn1)ncn2C)c1ccc(C2=CCN(C(=O)OC(C)(C)C)CC2)cc1OC(F)F] | 4-{3-Difluoromethoxy-4- [methyl-(1-methyl-1H- imidazo[4,5-c]pyridin-6- yl)-amino]-phenyl}-3,6- dihydro-2H-pyridine-1- carboxylic acid tert-butyl ester | Described above | 485 | 486 |
| 282 | 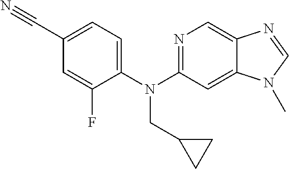 [SMILES: Cn1cnc2cnc(N(CC3CC3)c3ccc(C#N)cc3F)cc21] | 4-[Cyclopropylmethyl-(1- methyl-1H-imidazo[4,5- c]pyridin-6-yl)-amino]-3- fluoro-benzonitrile | A and E | 321 | 322 |
| 283 | 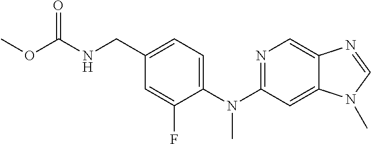 [SMILES: COC(=O)NCc1ccc(N(C)c2cc3c(cn2)ncn3C)c(F)c1] | {3-Fluoro-4-[methyl-(1- methyl-1H-imidazo[4,5- c]pyridin-6-yl)-amino]- benzyl}-carbamic acid methyl ester | Described above | 343 | 344 |
| 284 | 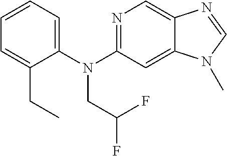 [SMILES: CCc1ccccc1N(CC(F)F)c1cc2c(cn1)ncn2C] | (2,2-Difluoro-ethyl)-(2- ethyl-phenyl)-(1-methyl- 1H-imidazo[4,5- c]pyridin-6-yl)-amine | E | 316 | 317 |
| 285 | 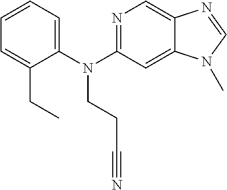 [SMILES: CCc1ccccc1N(CCC#N)c1cc2c(cn1)ncn2C] | 3-[(2-Ethyl-phenyl)-(1- methyl-1H-imidazo[4,5- c]pyridin-6-yl)-amino]- propionitrile | E | 305 | 306 |
| 286 | 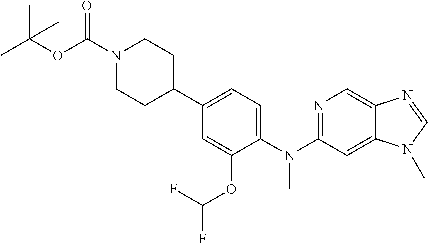 [SMILES: CN(c1cc2c(cn1)ncn2C)c1ccc(C2CCN(C(=O)OC(C)(C)C)CC2)cc1OC(F)F] | 4-{3-Difluoromethoxy-4- [methyl-(1-methyl-1H- imidazo[4,5-c]pyridin-6- yl)-amino]-phenyl}- piperidine-1-carboxylic acid tert-butyl ester | Described above | 487 | 488 |
| 287 | 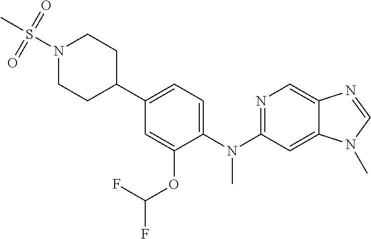 [SMILES: CN(c1cc2c(cn1)ncn2C)c1ccc(C2CCN(S(C)(=O)=O)CC2)cc1OC(F)F] | [2-Difluoromethoxy-4-(1- methanesulfonyl- piperidin-4-yl)-phenyl]- methyl-(1-methyl-1H- imidazo[4,5-c]pyridin-6- yl)-amine | Described above | 465 | 466 |
| 288 | 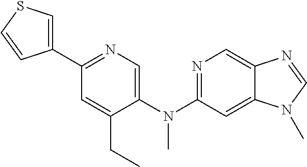 [SMILES: CCc1cc(-c2ccsc2)ncc1N(C)c1cc2c(cn1)ncn2C] | (4-Ethyl-6-thiophen-3-yl- pyridin-3-yl)-methyl-(1- methyl-1H-imidazo[4,5- c]pyridin-6-yl)-amine | Same as 289 | 349 | 350 |
| 289 | 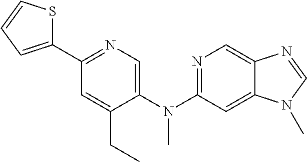 [SMILES: CCc1cc(-c2cccs2)ncc1N(C)c1cc2c(cn1)ncn2C] | (4-Ethyl-6-thiophen-2-yl- pyridin-3-yl)-methyl-(1- methyl-1H-imidazo[4,5- c]pyridin-6-yl)-amine | Described above | 349 | 350 |
| 290 | 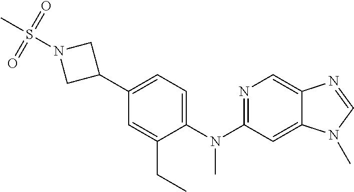 [SMILES: CCc1cc(C2CN(S(C)(=O)=O)C2)ccc1N(C)c1cc2c(cn1)ncn2C] | [2-Ethyl-4-(1- methanesulfonyl-azetidin- 3-yl)-phenyl]-methyl-(1- methyl-1H-imidazo[4,5- c]pyridin-6-yl)-amine | Described above | 399 | 400 |
| 291 | 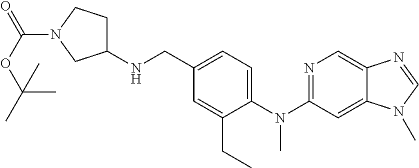 [SMILES: CCc1cc(CNC2CCN(C(=O)OC(C)(C)C)C2)ccc1N(C)c1cc2c(cn1)ncn2C] | 3-{3-Ethyl-4-[methyl-(1- methyl-1H-imidazo[4,5- c]pyridin-6-yl)-amino]- benzylamino}- pyrrolidine-1 carboxylic acid tert-butyl ester | Described above | 464 | 465 |
| 292 | 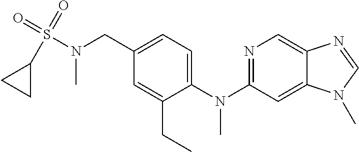 [SMILES: CCc1cc(CN(C)S(=O)(=O)C2CC2)ccc1N(C)c1cc2c(cn1)ncn2C] | Cyclopropanesulfonic acid {3-ethyl-4-[methyl- (1-methyl-1H- imidazo[4,5-c]pyridin-6- yl)-amino]-benzyl}- methyl-amide | Same as 261 | 413 | 414 |
| 293 | 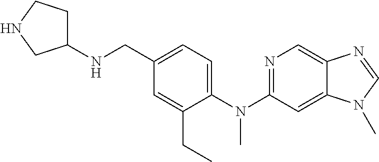 [SMILES: CCc1cc(CNC2CCNC2)ccc1N(C)c1cc2c(cn1)ncn2C] | [2-Ethyl-4-(pyrrolidin-3- ylaminomethyl)-phenyl]- methyl-(1-methyl-1H- imidazo[4,5-c]pyridin-6- yl)-amine | Described above | 364 | 365 |
| 294 | 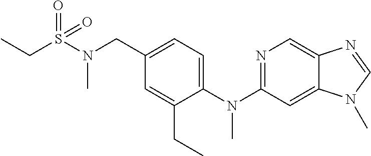 [SMILES: CCc1cc(CN(C)S(=O)(=O)CC)ccc1N(C)c1cc2c(cn1)ncn2C] | Ethanesulfonic acid {3- ethyl-4-[methyl-(1- methyl-1H-imidazo[4,5- c]pyridin-6-yl)-amino]- benzyl}-methyl-amide | Same as 261 | 401 | 402 |
| 295 | 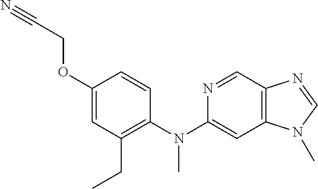 [SMILES: CCc1cc(OCC#N)ccc1N(C)c1cc2c(cn1)ncn2C] | 2-(3-ethyl-4-(methyl(1- methyl-1H-imidazo[4,5- c]pyridin-6- yl)amino)phenoxy)aceto- nitrile | Described above | 321 | 322 |
| 296 | 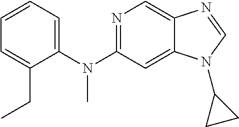 [SMILES: CCc1ccccc1N(C)c1cc2c(cn1)ncn2C1CC1] | 1-cyclopropyl-N-(2- ethylphenyl)-N-methyl- 1H-imidazo[4,5- c]pyridin-6-amine | Described above | 292 | 293 |
| 297 | 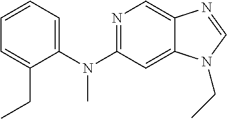 [SMILES: CCc1ccccc1N(C)c1cc2c(cn1)ncn2CC] | 1-ethyl-N-(2- ethylphenyl)-N-methyl- 1H-imidazo[4,5- c]pyridin-6-amine | Described above | 280 | 280.9 |
| 298 | 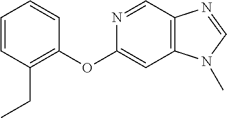 [SMILES: CCc1ccccc1Oc1cc2c(cn1)ncn2C] | 6-(2-Ethyl-phenoxy)-1- methyl-1H-imidazo[4,5- c]pyridine | D | 253 | 254 |
| 299 | 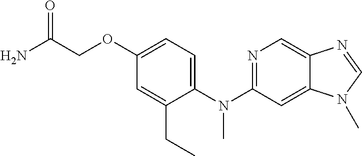 [SMILES: CCc1cc(OCC(N)=O)ccc1N(C)c1cc2c(cn1)ncn2C] | 2-{3-Ethyl-4-[methyl-(1- ethyl-1H-imidazo[4,5- c]pyridin-6-yl)-amino]- phenoxy}-acetamide | Described above | 339 | 340 |
| 300 | 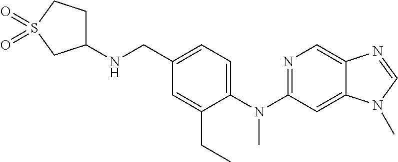 [SMILES: CCc1cc(CNC2CCS(=O)(=O)C2)ccc1N(C)c1cc2c(cn1)ncn2C] | {4-[(1,1-Dioxo- tetrahydro-thiophen-3- ylamino)-methyl]-2-ethyl- phenyl}-methyl-(1- methyl-1H-imidazo[4,5- c]pyridin-6-yl)-amine | Described above | 413 | 414 |
| 301 | 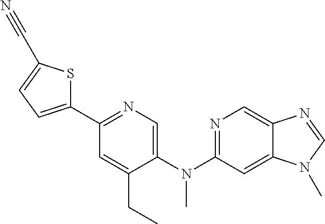 [SMILES: CCc1cc(-c2ccc(C#N)s2)ncc1N(C)c1cc2c(cn1)ncn2C] | 5-{4-Ethyl-5-[methyl-(1- methyl-1H-imidazo[4,5- c]pyridin-6-yl)-amino]- pyridin-2-yl}-thiophene- 2-carbonitrile | Described above | 374 | 375 |
| 302 | 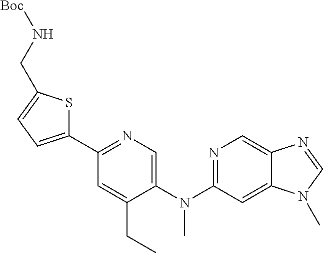 [SMILES: CCc1cc(-c2ccc(CNC(=O)OC(C)(C)C)s2)ncc1N(C)c1cc2c(cn1)ncn2C] | (5-{4-Ethyl-5-[methyl-(1- ethyl-1H-imidazo[4,5- c]pyridin-6-yl)-amino]- pyridin-2-yl}-thiophen-2- ylmethyl)-carbamic acid tert-butyl ester | Described above | 479 | 479 |
| 303 | 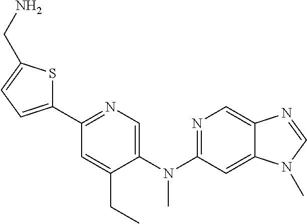 [SMILES: CCc1cc(-c2ccc(CN)s2)ncc1N(C)c1cc2c(cn1)ncn2C] | [6-(5-Aminomethyl- thiophen-2-yl)-4-ethyl- pyridin-3-yl]-methyl-(1- methyl-1H-imidazo[4,5- c]pyridin-6-yl)-amine | Described above | 378 | 379 |
| 304 | 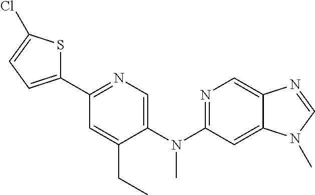 [SMILES: CCc1cc(-c2ccc(Cl)s2)ncc1N(C)c1cc2c(cn1)ncn2C] | [6-(5-Chloro-thiophen-2- yl)-4-ethyl-pyridin-3-yl]- methyl-(1-methyl-1H- imidazo[4,5-c]pyridin-6- yl)-amine | As 301 | 384 | 384 |
| 305 | 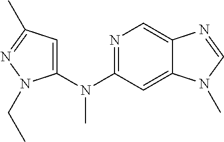 [SMILES: CCn1nc(C)cc1N(C)c1cc2c(cn1)ncn2C] | (2-Ethyl-5-methyl-2H- pyrazol-3-yl)-methyl-(1- methyl-1H-imidazo[4,5- c]pyridin-6-yl)-amine | As 302 | 270 | 271 |
| 306 | 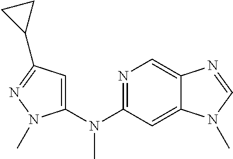 [SMILES: CN(c1cc2c(cn1)ncn2C)c1cc(C2CC2)nn1C] | (5-Cyclopropyl-2-methyl- 2H-pyrazol-3-yl)-methyl- (1-methyl-1H- imidazo[4,5-c]pyridin-6- yl)-amine | As 302 | 296 | 297 |
| 307 | 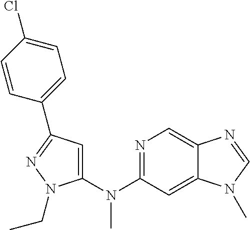 [SMILES: CCn1nc(-c2ccc(Cl)cc2)cc1N(C)c1cc2c(cn1)ncn2C] | [5-(4-Chloro-phenyl)-2- ethyl-2H-pyrazol-3-yl]- methyl-(1-methyl-1H- imidazo[4,5-c]pyridin-6- yl)-amine | Described above | 367 | 367 |
| 308 | 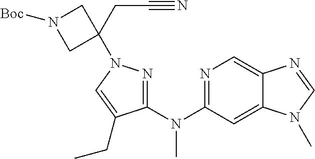 [SMILES: CCc1cn(C2(CC#N)CN(C(=O)OC(C)(C)C)C2)nc1N(C)c1cc2c(cn1)ncn2C] | 3-Cyanomethyl-3-{4- ethyl-3-[methyl-(1- methyl-1H-imidazo[4,5- c]pyridin-6-yl)-amino]- pyrazol-1-yl}-azetidine-1- carboxylic acid tert-butyl ester | Described above | 450 | 451 |
| 309 | 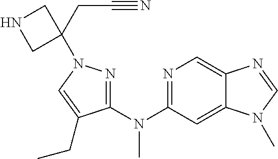 [SMILES: CCc1cn(C2(CC#N)CNC2)nc1N(C)c1cc2c(cn1)ncn2C] | (3-{4-Ethyl-3-[methyl-(1- methyl-1H-imidazo[4,5- c]pyridin-6-yl)-amino]- pyrazol-1-yl}-azetidin-3- yl)-acetonitrile | As 303 | 350 | 351 |
| 310 | 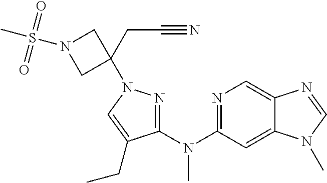 [SMILES: CCc1cn(C2(CC#N)CN(S(C)(=O)=O)C2)nc1N(C)c1cc2c(cn1)ncn2C] | (3-{4-Ethyl-3-[methyl-(1- methyl-1H-imidazo[4,5- c]pyridin-6-yl)-amino]- pyrazol-1-yl}-1- methanesulfonyl-azetidin- 3-yl)-acetonitrile | Described above | 428 | 429 |
| 311 | 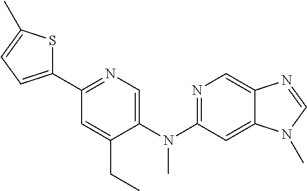 [SMILES: CCc1cc(-c2ccc(C)s2)ncc1N(C)c1cc2c(cn1)ncn2C] | [4-Ethyl-6-(5-methyl- thiophen-2-yl)-pyridin-3- yl]-methyl-(1-methyl-1H- imidazo[4,5-c]pyridin-6- yl)-amine | Described above | 363 | 364 |
| 312 | 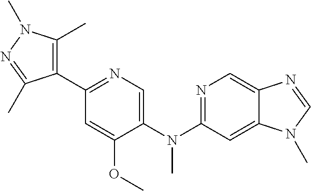 [SMILES: COc1cc(-c2c(C)nn(C)c2C)ncc1N(C)c1cc2c(cn1)ncn2C] | [4-Methoxy-6-(1,3,5- trimethyl-1H-pyrazol-4- yl)-pyridin-3-yl]-methyl- (1-methyl-1H- imidazo[4,5-c]pyridin-6- yl)-amine | Described above | 377 | 379 |
| 313 | 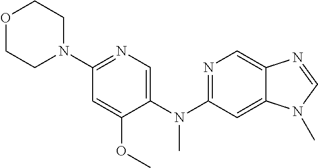 [SMILES: COc1cc(N2CCOCC2)ncc1N(C)c1cc2c(cn1)ncn2C] | (4-Methoxy-6-morpholin- 4-yl-pyridin-3-yl)-methyl- (1-methyl-1H- imidazo[4,5-c]pyridin-6- yl)-amine | Described above | 353 | 355 |
| 314 | 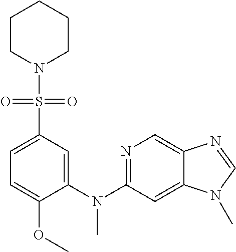 [SMILES: COc1ccc(S(=O)(=O)N2CCCCC2)cc1N(C)c1cc2c(cn1)ncn2C] | [2-Methoxy-5- (piperidine-1-sulfonyl)- phenyl]-methyl-(1- methyl-1H-imidazo[4,5- c]pyridin-6-yl)-amine | Described above | 415 | 417 |
| 315 | 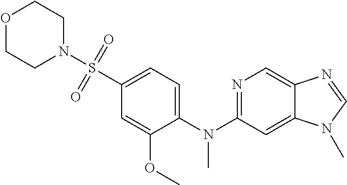 [SMILES: COc1cc(S(=O)(=O)N2CCOCC2)ccc1N(C)c1cc2c(cn1)ncn2C] | [2-Methoxy-4- (morpholine-4-sulfonyl)- phenyl]-methyl-(1- methyl-1H-imidazo[4,5- c]pyridin-6-yl)-amine | Described above | 417 | 419 |
| 316 | 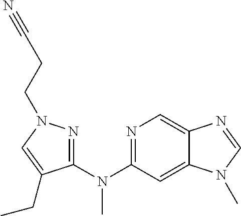 [SMILES: CCc1cn(CCC#N)nc1N(C)c1cc2c(cn1)ncn2C] | 3-{4-Ethyl-3-[methyl-(1- methyl-1H-imidazo[4,5- c]pyridin-6-yl)-amino]- pyrazol-1-yl}- propionitrile | Described above | 309 | 310 |
| 317 | 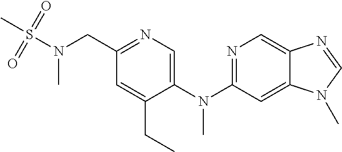 [SMILES: CCc1cc(CN(C)S(C)(=O)=O)ncc1N(C)c1cc2c(cn1)ncn2C] | N-{4-Ethyl-5-[methyl-(1- methyl-1H-imidazo[4,5- c]pyridin-6-yl)-amino]- pyridin-2-ylmethyl}-N- methyl-methane- sulfonamide | Described above | 388 | 389 |
| 318 | 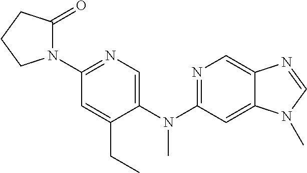 [SMILES: CCc1cc(N2CCCC2=O)ncc1N(C)c1cc2c(cn1)ncn2C] | 1-{4-Ethyl-5-[methyl-(1- methyl-1H-imidazo[4,5- c]pyridin-6-yl)-amino]- pyridin-2-yl}-pyrrolidin- 2-one | Described above | 350 | 351 |
| 319 | 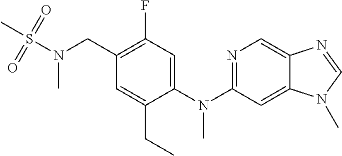 [SMILES: CCc1cc(CN(C)S(C)(=O)=O)c(F)cc1N(C)c1cc2c(cn1)ncn2C] | N-{5-Ethyl-2-fluoro-4- [methyl-(1-methyl-1H- imidazo[4,5-c]pyridin-6- yl)-amino]-benzyl}-N- methyl- methanesulfonamide | Described above | 405 | 406 |
| 320 | 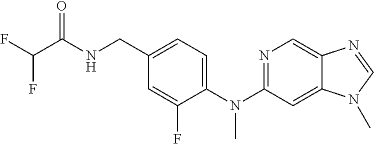 [SMILES: CN(c1cc2c(cn1)ncn2C)c1ccc(CNC(=O)C(F)F)cc1F] | 2,2-Difluoro-N-{3-fluoro- 4-[methyl-(1-methyl-1H- imidazo[4,5-c]pyridin-6- yl)-amino]-benzyl}- acetamide | Described above | 363 | 364 |
| 321 | 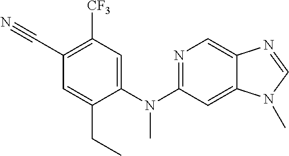 [SMILES: CCc1cc(C#N)c(C(F)(F)F)cc1N(C)c1cc2c(cn1)ncn2C] | 5-Ethyl-4-[methyl-(1- methyl-1H-imidazo[4,5- c]pyridin-6-yl)-amino]-2- trifluoromethyl- benzonitrile | Described above | 359 | 360 |
| 322 | 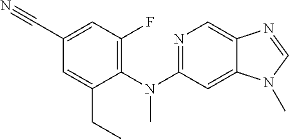 [SMILES: CCc1cc(C#N)cc(F)c1N(C)c1cc2c(cn1)ncn2C] | 3-Ethyl-5-fluoro-4- [methyl-(1-methyl-1H- imidazo[4,5-c]pyridin-6- yl)-amino]-benzonitrile | Described above | 309 | 310 |
| 323 | 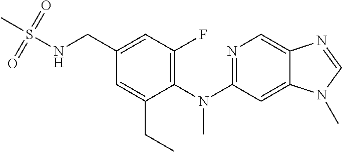 [SMILES: CCc1cc(CNS(C)(=O)=O)cc(F)c1N(C)c1cc2c(cn1)ncn2C] | N-{3-Ethyl-5-fluoro-4- [methyl-(1-methyl-1H- imidazo[4,5-c]pyridin-6- yl)-amino]-benzyl}- methanesulfonamide | Described above | 491 | 492 |
| 324 | 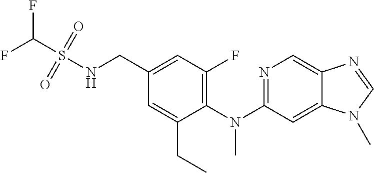 [SMILES: CCc1cc(CNS(=O)(=O)C(F)F)cc(F)c1N(C)c1cc2c(cn1)ncn2C] | N-{3-Ethyl-5-fluoro-4- [methyl-(1-methyl-1H- imidazo[4,5-c]pyridin-6- yl)-amino]-benzyl}-C,C- difluoro- methanesulfonamide | Described above | 427 | 428 |
| 325 | 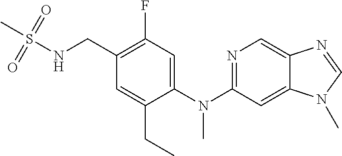 [SMILES: CCc1cc(CNS(C)(=O)=O)c(F)cc1N(C)c1cc2c(cn1)ncn2C] | N-{5-Ethyl-2-fluoro-4- [methyl-(1-methyl-1H- imidazo[4,5-c]pyridin-6- yl)-amino]-benzyl}- methanesulfonamide | Described above | 391 | 392 |
| 326 | 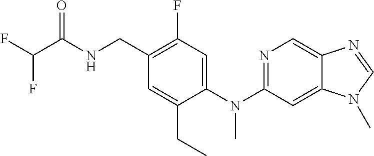 [SMILES: CCc1cc(CNC(=O)C(F)F)c(F)cc1N(C)c1cc2c(cn1)ncn2C] | N-{5-Ethyl-2-fluoro-4- [methyl-(1-methyl-1H- imidazo[4,5-c]pyridin-6- yl)-amino]-benzyl}-2,2- difluoro-acetamide | Described above | 391 | 392 |
| 327 | 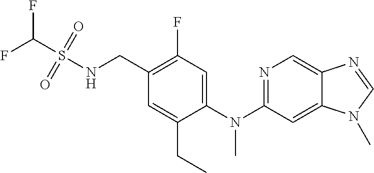 [SMILES: CCc1cc(CNS(=O)(=O)C(F)F)c(F)cc1N(C)c1cc2c(cn1)ncn2C] | N-{5-Ethyl-2-fluoro-4- [methyl-(1-methyl-1H- imidazo[4,5-c]pyridin-6- yl)-amino]-benzyl}-C,C- difluoro- methanesulfonamide | Described above | 427 | 428 |
| 328 | 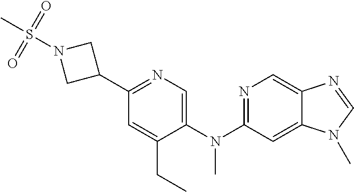 [SMILES: CCc1cc(C2CN(S(C)(=O)=O)C2)ncc1N(C)c1cc2c(cn1)ncn2C] | [4-Ethyl-6-(1- methanesulfonyl-azetidin- 3-yl)-pyridin-3-yl]- methyl-(1-methyl-1H- imidazo[4,5-c]pyridin-6- yl)-amine | Described above | 400 | 401 |
| 329 | 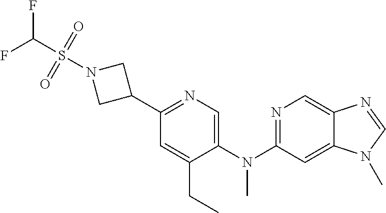 [SMILES: CCc1cc(C2CN(S(=O)(=O)C(F)F)C2)ncc1N(C)c1cc2c(cn1)ncn2C] | {6-[1-(Difluoro- methanesulfonyl)- azetidin-3-yl]-4-ethyl- pyridin-3-yl}-methyl-(1- methyl-1H-imidazo[4,5- c]pyridin-6-yl)-amine | Described above | 436 | 437 |
| 330 | 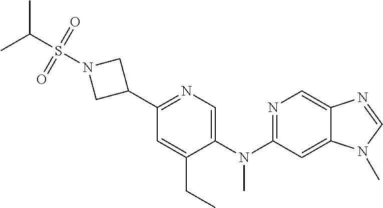 [SMILES: CCc1cc(C2CN(S(=O)(=O)C(C)C)C2)ncc1N(C)c1cc2c(cn1)ncn2C] | {4-Ethyl-6-[1-(propane-2- sulfonyl)-azetidin-3-yl]- pyridin-3-yl}-methyl-(1- methyl-1H-imidazo[4,5- c]pyridin-6-yl)-amine | Described above | 429 | 429 |
| 331 | 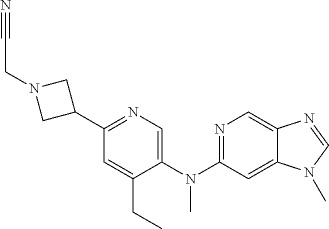 [SMILES: CCc1cc(C2CN(CC#N)C2)ncc1N(C)c1cc2c(cn1)ncn2C] | (3-{4-Ethyl-5-[methyl-(1- methyl-1H-imidazo[4,5- c]pyridin-6-yl)-amino]- pyridin-2-yl}-azetidin-1- yl)-acetonitrile | Described above | 361 | 362 |
| 332 | 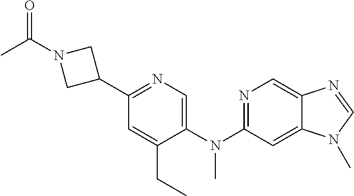 [SMILES: CCc1cc(C2CN(C(C)=O)C2)ncc1N(C)c1cc2c(cn1)ncn2C] | 1-(3-{4-Ethyl-5-[methyl- (1-methyl-1H- imidazo[4,5-c]pyridin-6- yl)-amino]-pyridin-2-yl}- azetidin-1-yl)-ethanone | Described above | 364 | 365 |
| 333 | 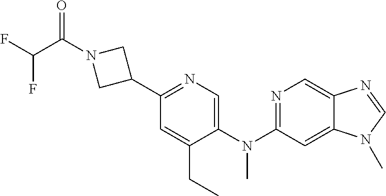 [SMILES: CCc1cc(C2CN(C(=O)C(F)F)C2)ncc1N(C)c1cc2c(cn1)ncn2C] | 1-(3-{4-Ethyl-5-[methyl- (1-methyl-1H- imidazo[4,5-c]pyridin-6- yl)-amino]-pyridin-2-yl}- azetidin-1-yl)-2,2- difluoro-ethanone | Described above | 400 | 401 |
| 334 | 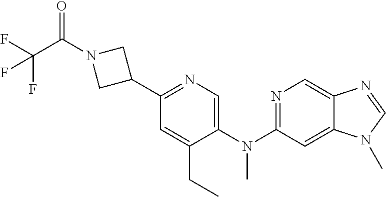 [SMILES: CCc1cc(C2CN(C(=O)C(F)(F)F)C2)ncc1N(C)c1cc2c(cn1)ncn2C] | 1-(3-{4-Ethyl-5-[methyl- (1-methyl-1H- imidazo[4,5-c]pyridin-6- yl)-amino]-pyridin-2-yl}- azetidin-1-yl)-2,2,2- trifluoro-ethanone | Described above | 418 | 419 |
| 335 | 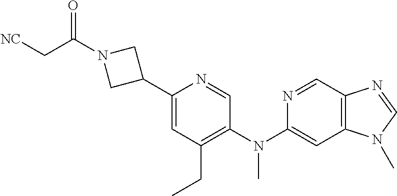 [SMILES: CCc1cc(C2CN(C(=O)CC#N)C2)ncc1N(C)c1cc2c(cn1)ncn2C] | 3-(3-{4-Ethyl-5-[methyl- (1-methyl-1H- imidazo[4,5-c]pyridin-6- yl)-amino]-pyridin-2-yl}- azetidin-1-yl)-3-oxo- propionitrile | Described above | 389 | 390 |
| 336 | 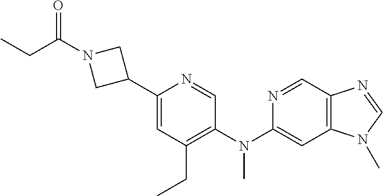 [SMILES: CCC(=O)N1CC(c2cc(CC)c(N(C)c3cc4c(cn3)ncn4C)cn2)C1] | 1-(3-{4-Ethyl-5-[methyl- (1-methyl-1H- imidazo[4,5-c]pyridin-6- yl)-amino]-pyridin-2-yl}- azetidin-1-yl)-propan-1- one | Described above | 378 | 379 |
| 337 | 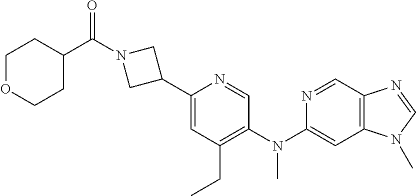 [SMILES: CCc1cc(C2CN(C(=O)C3CCOCC3)C2)ncc1N(C)c1cc2c(cn1)ncn2C] | (3-{4-Ethyl-5-[methyl-(1- methyl-1H-imidazo[4,5- c]pyridin-6-yl)-amino]- pyridin-2-yl}-azetidin-1- yl)-(tetrahydro-pyran-4- yl)-methanone | Described above | 434 | 435 |
| 338 | 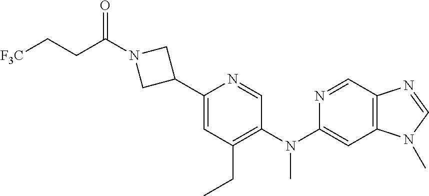 [SMILES: CCc1cc(C2CN(C(=O)CCC(F)(F)F)C2)ncc1N(C)c1cc2c(cn1)ncn2C] | 1-(3-{4-Ethyl-5-[methyl- (1-methyl-1H- imidazo[4,5-c]pyridin-6- yl)-amino]-pyridin-2-yl}- azetidin-1-yl)-4,4,4- trifluoro-butan-1-one | Described above | 446 | 447 |
| 339 | 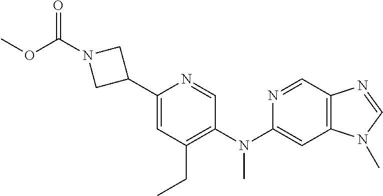 [SMILES: CCc1cc(C2CN(C(=O)OC)C2)ncc1N(C)c1cc2c(cn1)ncn2C] | 3-{4-Ethyl-5-[methyl-(1- methyl-1H-imidazo[4,5- c]pyridin-6-yl)-amino]- pyridin-2-yl}-azetidine-1- carboxylic acid methyl ester | Described above | 380 | 381 |
| 340 | 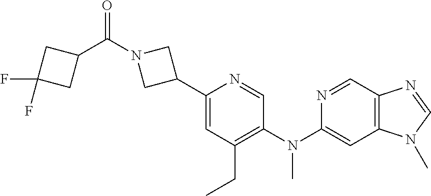 [SMILES: CCc1cc(C2CN(C(=O)C3CC(F)(F)C3)C2)ncc1N(C)c1cc2c(cn1)ncn2C] | (3,3-Difluoro-cyclobutyl)- (3-{4-ethyl-5-[methyl-(1- methyl-1H-imidazo[4,5- c]pyridin-6-yl)-amino]- pyridin-2-yl}-azetidin-1- yl)-methanone | Described above | 440 | 441 |
| 341 | 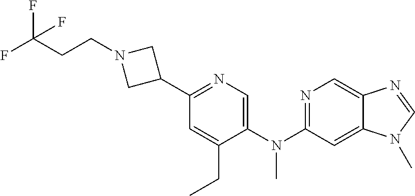 [SMILES: CCc1cc(C2CN(CCC(F)(F)F)C2)ncc1N(C)c1cc2c(cn1)ncn2C] | {4-Ethyl-6-[1-(3,3,3- trifluoro-propyl)-azetidin- 3-yl]-pyridin-3-yl}- methyl-(1-methyl-1H- imidazo[4,5-c]pyridin-6- yl)-amine | Described above | 418 | 419 |
| 342 | 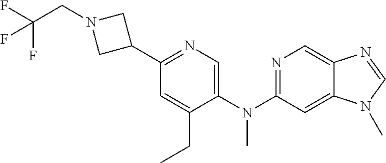 [SMILES: CCc1cc(C2CN(CC(F)(F)F)C2)ncc1N(C)c1cc2c(cn1)ncn2C] | {4-Ethyl-6-[1-(2,2,2- trifluoro-ethyl)-azetidin- 3-yl]-pyridin-3-yl}- methyl-(1-methyl-1H- imidazo[4,5-c]pyridin-6- yl)-amine | Described above | 404 | 405 |
| 343 | 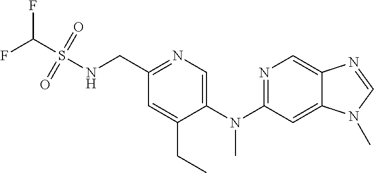 [SMILES: CCc1cc(CNS(=O)(=O)C(F)F)ncc1N(C)c1cc2c(cn1)ncn2C] | N-{4-Ethyl-5-[methyl-(1- methyl-1H-imidazo[4,5- c]pyridin-6-yl)-amino]- pyridin-2-ylmethyl}-C,C- difluoro-methane- sulfonamide | Described above | 410 | 411 |
| 344 | 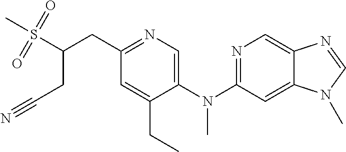 [SMILES: CCc1cc(CC(CC#N)S(C)(=O)=O)ncc1N(C)c1cc2c(cn1)ncn2C] | N-Cyanomethyl-N-{4- ethyl-5-[methyl-(1- methyl-1H-imidazo[4,5- c]pyridin-6-yl)-amino]- pyridin-2-ylmethyl}- methanesulfonamide | Described above | 413 | 415 |
| 345 | 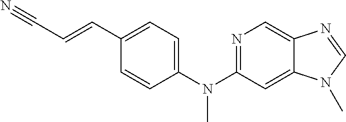 [SMILES: CN(c1ccc(/C=C/C#N)cc1)c1cc2c(cn1)ncn2C] | (E)-3-{4-[Methyl-(1- methyl-1H-imidazo[4,5- c]pyridin-6-yl)-amino]- phenyl}-acrylonitrile | Described above | 289 | 290 |
| 346 | 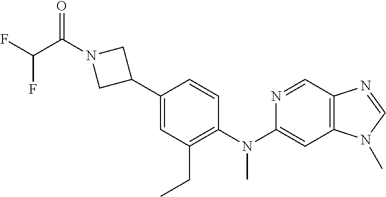 [SMILES: CCc1cc(C2CN(C(=O)C(F)F)C2)ccc1N(C)c1cc2c(cn1)ncn2C] | 1-(3-{3-Ethyl-4-[methyl- (1-methyl-1H- imidazo[4,5-c]pyridin-6- yl)-amino]-phenyl}- azetidin-1-yl)-2,2- difluoro-ethanone | Described above | 399 | 400 |
| 347 | 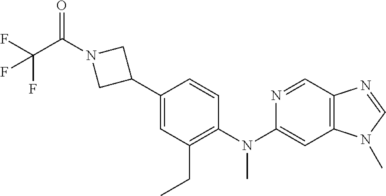 [SMILES: CCc1cc(C2CN(C(=O)C(F)(F)F)C2)ccc1N(C)c1cc2c(cn1)ncn2C] | 1-(3-{3-Ethyl-4-[methyl- (1-methyl-1H- imidazo[4,5-c]pyridin-6- yl)-amino]-phenyl}- azetidin-1-yl)-2,2,2- trifluoro-ethanone | Described above | 417 | 418 |
| 348 | 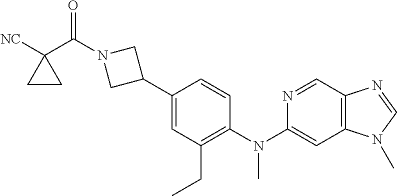 [SMILES: CCc1cc(C2CN(C(=O)C3(C#N)CC3)C2)ccc1N(C)c1cc2c(cn1)ncn2C] | 1-(3-{3-Ethyl-4-[methyl- (1-methyl-1H- imidazo[4,5-c]pyridin-6- yl)-amino]-phenyl}- azetidine-1-carbonyl)- cyclopropanecarbonitrile | Described above | 414 | 415 |
| 349 | 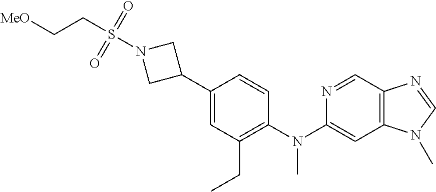 [SMILES: CCc1cc(C2CN(S(=O)(=O)CCOC)C2)ccc1N(C)c1cc2c(cn1)ncn2C] | {2-Ethyl-4-[1-(2- methoxy-ethanesulfonyl)- azetidin-3-yl]-phenyl}- methyl-(1-methyl-1H- imidazo[4,5-c]pyridin-6- yl)-amine | Described above | 443 | 444 |
| 350 | 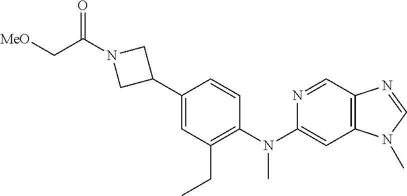 [SMILES: CCc1cc(C2CN(C(=O)COC)C2)ccc1N(C)c1cc2c(cn1)ncn2C] | 1-(3-{3-Ethyl-4-[methyl- (1-methyl-1H- imidazo[4,5-c]pyridin-6- yl)-amino]-phenyl}- azetidin-1-yl)-2-methoxy- ethanone | Described above | 393 | 394 |
| 351 | 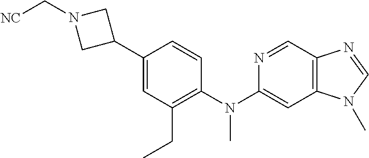 [SMILES: CCc1cc(C2CN(CC#N)C2)ccc1N(C)c1cc2c(cn1)ncn2C] | (3-{3-Ethyl-4-[methyl-(1- methyl-1H-imidazo[4,5- c]pyridin-6-yl)-amino]- phenyl}-azetidin-1-yl)- acetonitrile | Described above | 360 | 361 |
| 352 | 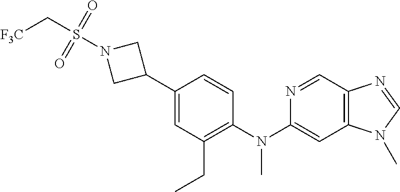 [SMILES: CCc1cc(C2CN(S(=O)(=O)CC(F)(F)F)C2)ccc1N(C)c1cc2c(cn1)ncn2C] | {2-Ethyl-4-[1-(2,2,2- trifluoro-ethanesulfonyl)- azetidin-3-yl]-phenyl}- methyl-(1-methyl-1H- imidazo[4,5-c]pyridin-6- yl)-amine | Described above | 467 | 468 |
| 353 | 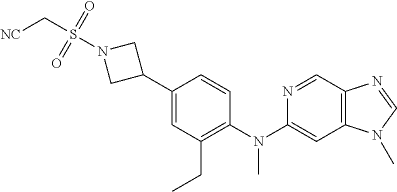 [SMILES: CCc1cc(C2CN(S(=O)(=O)CC#N)C2)ccc1N(C)c1cc2c(cn1)ncn2C] | (3-{3-Ethyl-4-[methyl-(1- methyl-1H-imidazo[4,5- c]pyridin-6-yl)-amino]- phenyl}-azetidine-1- sulfonyl)-acetonitrile | Described above | 424 | 425 |
| 354 | 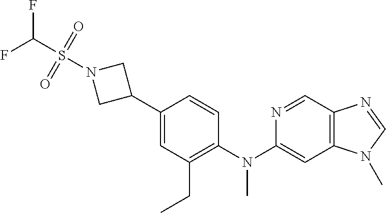 [SMILES: CCc1cc(C2CN(S(=O)(=O)C(F)F)C2)ccc1N(C)c1cc2c(cn1)ncn2C] | {4-[1-(Difluoro- methanesulfonyl)- azetidin-3-yl]-2-ethyl- phenyl}-methyl-(1- methyl-1H-imidazo[4,5- c]pyridin-6-yl)-amine | Described above | 435 | 436 |
| 355 | 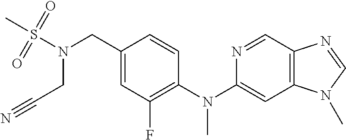 [SMILES: CN(c1cc2c(cn1)ncn2C)c1ccc(CN(CC#N)S(C)(=O)=O)cc1F] | N-Cyanomethyl-N-{3- fluoro-4-[methyl-(1- methyl-1H-imidazo[4,5- c]pyridin-6-yl)-amino]- benzyl}- methanesulfonamide | Described above | 402 | 403 |
| 356 | 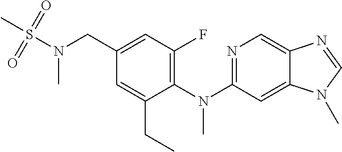 [SMILES: CCc1cc(CN(C)S(C)(=O)=O)cc(F)c1N(C)c1cc2c(cn1)ncn2C] | N-{3-Ethyl-5-fluoro-4- [methyl-(1-methyl-1H- imidazo[4,5-c]pyridin-6- yl)-amino]-benzyl}-N- methyl- methanesulfonamide | Described above | 405 | 406 |
| 357 | 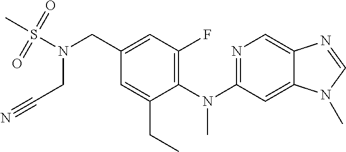 [SMILES: CCc1cc(CN(CC#N)S(C)(=O)=O)cc(F)c1N(C)c1cc2c(cn1)ncn2C] | N-Cyanomethyl-N-{3- ethyl-5-fluoro-4-[methyl- (1-methyl-1H- imidazo[4,5-c]pyridin-6- yl)-amino]-benzyl}- methanesulfonamide | Described above | 430 | 431 |
| 358 | 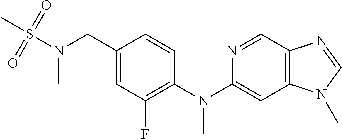 [SMILES: CN(c1cc2c(cn1)ncn2C)c1ccc(CN(C)S(C)(=O)=O)cc1F] | N-{3-Fluoro-4-[methyl- (1-methyl-1H- imidazo[4,5-c]pyridin-6- yl)-amino]-benzyl}-N- methyl- methanesulfonamide | Described above | 377 | 378 |
| 359 | 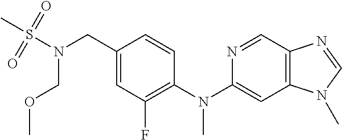 [SMILES: COCN(Cc1ccc(N(C)c2cc3c(cn2)ncn3C)c(F)c1)S(C)(=O)=O] | N-{3-Fluoro-4-[methyl- (1-methyl-1H- imidazo[4,5-c]pyridin-6- yl)-amino]-benzyl}-N- methoxymethyl- methanesulfonamide | Described above | 407 | 408 |
| 360 | 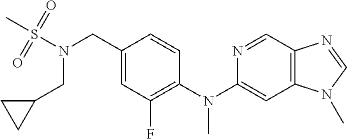 [SMILES: CN(c1cc2c(cn1)ncn2C)c1ccc(CN(CC2CC2)S(C)(=O)=O)cc1F] | N-Cyclopropylmethyl-N- {3-fluoro-4-[methyl-(1- methyl-1H-imidazo[4,5- c]pyridin-6-yl)-amino]- benzyl}-methane- sulfonamide | Described above | 417 | 418 |
| 362 | 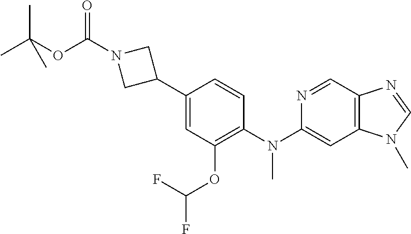 [SMILES: CN(c1cc2c(cn1)ncn2C)c1ccc(C2CN(C(=O)OC(C)(C)C)C2)cc1OC(F)F] | 3-{3-Difluoromethoxy-4- [methyl-(1-methyl-1H- imidazo[4,5-c]pyridin-6- yl)-amino]-phenyl}- azetidine-1-carboxylic acid tert-butyl ester | Described above | 460 | 460 |
| 363 | 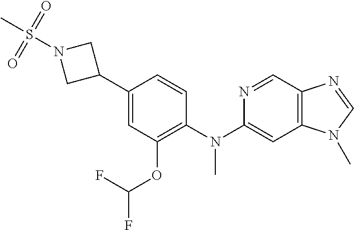 [SMILES: CN(c1cc2c(cn1)ncn2C)c1ccc(C2CN(S(C)(=O)=O)C2)cc1OC(F)F] | [2-Difluoromethoxy-4-(1- methanesulfonyl-azetidin- 3-yl)-phenyl]-methyl-(1- methyl-1H-imidazo[4,5- c]pyridin-6-yl)-amine | Described above | 437 | 438 |
| 364 | 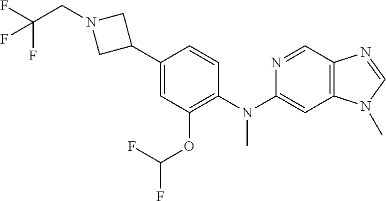 [SMILES: CN(c1cc2c(cn1)ncn2C)c1ccc(C2CN(CC(F)(F)F)C2)cc1OC(F)F] | {2-Difluoromethoxy-4-[1- (2,2,2-trifluoro-ethyl)- azetidin-3-yl]-phenyl}- methyl-(1-methyl-1H- imidazo[4,5-c]pyridin-6- yl)-amine | Described above | 441 | 442 |
| 365 | 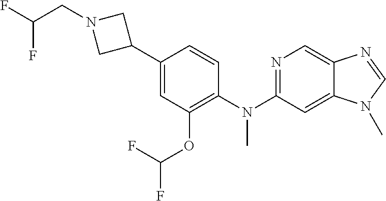 [SMILES: CN(c1cc2c(cn1)ncn2C)c1ccc(C2CN(CC(F)F)C2)cc1OC(F)F] | {4-[1-(2,2-Difluoro- ethyl)-azetidin-3-yl]-2- difluoromethoxy-phenyl}- methyl-(1-methyl-1H- imidazo[4,5-c]pyridin-6- yl)-amine | Described above | 423 | 424 |
| 366 | 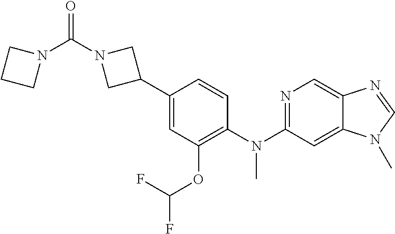 [SMILES: CN(c1cc2c(cn1)ncn2C)c1ccc(C2CN(C(=O)N3CCC3)C2)cc1OC(F)F] | Azetidin-1-yl-(3-{3- difluoromethoxy-4- [methyl-(1-methyl-1H- imidazo[4,5-c]pyridin-6- yl)-amino]-phenyl}- azetidin-1-yl)-methanone | Described above | 442 | 443 |
| 367 | 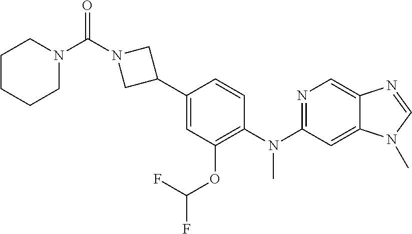 [SMILES: CN(c1cc2c(cn1)ncn2C)c1ccc(C2CN(C(=O)N3CCCCC3)C2)cc1OC(F)F] | (3-{3-Difluoromethoxy-4- [methyl-(1-methyl-1H- imidazo[4,5-c]pyridin-6- yl)-amino]-phenyl}- azetidin-1-yl)-piperidin-1- yl-methanone | Described above | 470 | 471 |
| 368 | 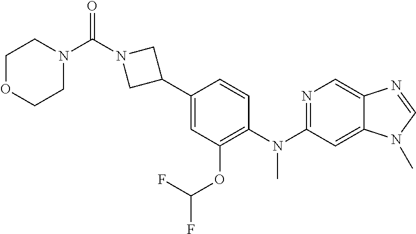 [SMILES: CN(c1cc2c(cn1)ncn2C)c1ccc(C2CN(C(=O)N3CCOCC3)C2)cc1OC(F)F] | (3-{3-Difluoromethoxy-4- [methyl-(1-methyl-1H- imidazo[4,5-c]pyridin-6- yl)-amino]-phenyl}- azetidin-1-yl)-morpholin- 4-yl-methanone | Described above | 472 | 473 |
| 369 | 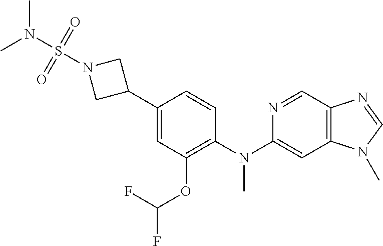 [SMILES: CN(c1cc2c(cn1)ncn2C)c1ccc(C2CN(S(=O)(=O)N(C)C)C2)cc1OC(F)F] | 3-{3-Difluoromethoxy-4- [methyl-(1-methyl-1H- imidazo[4,5-c]pyridin-6- yl)-amino]-phenyl}- azetidine-1-sulfonic acid dimethylamide | Described above | 466 | 467 |
| 370 | 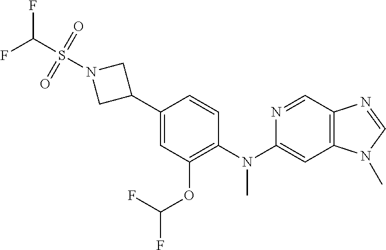 [SMILES: CN(c1cc2c(cn1)ncn2C)c1ccc(C2CN(S(=O)(=O)C(F)F)C2)cc1OC(F)F] | {4-[1-(Difluoro- methanesulfonyl)- azetidin-3-yl]-2- difluoromethoxy phenyl}- methyl-(1-methyl-1H- imidazo[4,5-c]pyridin-6- yl)-amine | Described above | 473 | 474 |
| 371 | 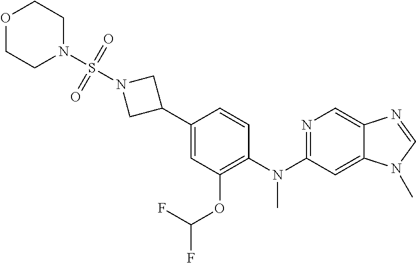 [SMILES: CN(c1cc2c(cn1)ncn2C)c1ccc(C2CN(S(=O)(=O)N3CCOCC3)C2)cc1OC(F)F] | {2-Difluoromethoxy-4-[1- (morpholine-4-sulfonyl)- azetidin-3-yl]-phenyl}- methyl-(1-methyl-1H- imidazo[4,5-c]pyridin-6- yl)-amine | Described above | 508 | 509 |
| 372 | 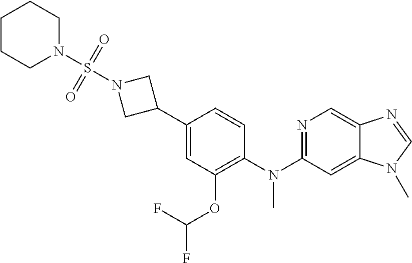 [SMILES: CN(c1cc2c(cn1)ncn2C)c1ccc(C2CN(S(=O)(=O)N3CCCCC3)C2)cc1OC(F)F] | {2-Difluoromethoxy-4-[1- (piperidine-1-sulfonyl)- azetidin-3-yl]-phenyl}- methyl-(1-methyl-1H- imidazo[4,5-c]pyridin-6- yl)-amine | Described above | 507 | 507 |
| 373 | 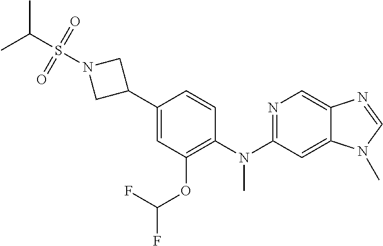 [SMILES: CC(C)S(=O)(=O)N1CC(c2ccc(N(C)c3cc4c(cn3)ncn4C)c(OC(F)F)c2)C1] | {2-Difluoromethoxy-4-[1- (propane-2-sulfonyl)- azetidin-3-yl]-phenyl}- methyl-(1-methyl-1H- imidazo[4,5-c]pyridin-6- yl)-amine | Described above | 465 | 466 |
| 374 | 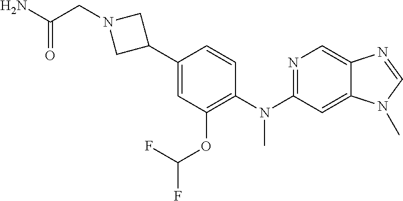 [SMILES: CN(c1cc2c(cn1)ncn2C)c1ccc(C2CN(CC(N)=O)C2)cc1OC(F)F] | 2-(3-{3-Difluoromethoxy- 4-[methyl-(1-methyl-1H- imidazo[4,5-c]pyridin-6- yl)-amino]-phenyl}- azetidin-1-yl)-acetamide | Described above | 416 | 417 |
| 375 | 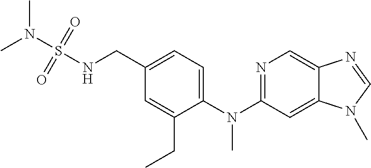 [SMILES: CCc1cc(CNS(=O)(=O)N(C)C)ccc1N(C)c1cc2c(cn1)ncn2C] | Dimethyl amino-N-{3- Ethyl-4-[methyl-(1- methyl-1H-imidazo[4,5- c]pyridin-6-yl)-amino]- benzyl}-sulfonamide | Described above | 402 | 403 |
| 376 | 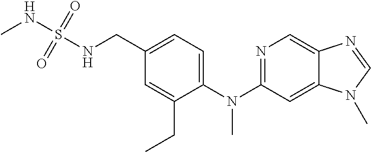 [SMILES: CCc1cc(CNS(=O)(=O)NC)ccc1N(C)c1cc2c(cn1)ncn2C] | Methyl amino-N-{3- Ethyl-4-[methyl-(1- methyl-1H-imidazo[4,5- c]pyridin-6-yl)-amino]- benzyl}-sulfonamide | Described above | 388 | 389 |
| 377 | 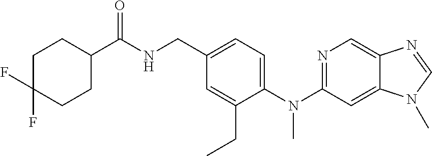 [SMILES: CCc1cc(CNC(=O)C2CCC(F)(F)CC2)ccc1N(C)c1cc2c(cn1)ncn2C] | 4,4-Difluoro- cyclohexanecarboxylic acid 3-ethyl-4-[methyl-(1- methyl-1H-imidazo[4,5- c]pyridin-6-yl)-amino]- benzylamide | Described above | 441 | 442 |
| 378 | 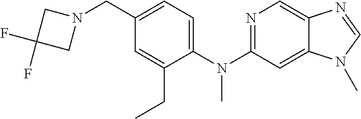 [SMILES: CCc1cc(CN2CC(F)(F)C2)ccc1N(C)c1cc2c(cn1)ncn2C] | [4-(3,3-Difluoro-azetidin- 1-ylmethyl)-2-ethyl- phenyl]-methyl-(1- methyl-1H-imidazo[4,5- c]pyridin-6-yl)-amine | Described above | 371 | 372 |
| 379 | 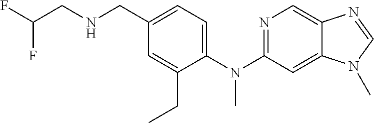 [SMILES: CCc1cc(CNCC(F)F)ccc1N(C)c1cc2c(cn1)ncn2C] | {4-[(2,2-Difluoro- ethylamino)-methyl]-2- ethyl-phenyl}-methyl-(1- methyl-1H-imidazo[4,5- c]pyridin-6-yl)-amine | Described above | 359 | 360 |
| 380 | 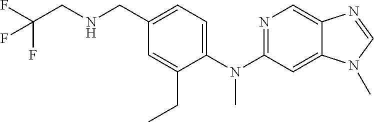 [SMILES: CCc1cc(CNCC(F)(F)F)ccc1N(C)c1cc2c(cn1)ncn2C] | {2-Ethyl-4-[(2,2,2- trifluoro-ethylamino)- methyl]-phenyl}-methyl- (1-methyl-1H- imidazo[4,5-c]pyridin-6- yl)-amine | Described above | 377 | 378 |
| 381 | 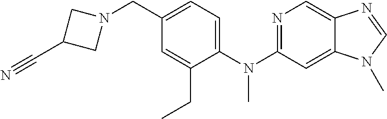 [SMILES: CCc1cc(CN2CC(C#N)C2)ccc1N(C)c1cc2c(cn1)ncn2C] | 1-{3-Ethyl-4-[methyl-(1- methyl-1H-imidazo[4,5- c]pyridin-6-yl)-amino]- benzyl}-azetidine-3- carbonitrile | Described above | 360 | 361 |
| 382 | 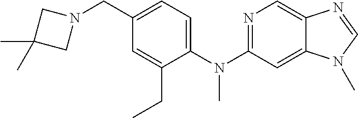 [SMILES: CCc1cc(CN2CC(C)(C)C2)ccc1N(C)c1cc2c(cn1)ncn2C] | [4-(3,3-Dimethyl- azetidin-1-ylmethyl)-2- ethyl-phenyl]-methyl-(1- methyl-1H-imidazo[4,5- c]pyridin-6-yl)-amine | Described above | 363 | 364 |
| 383 | 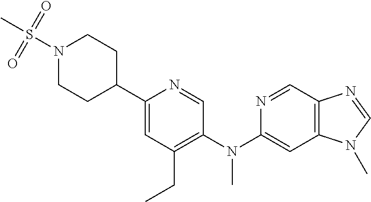 [SMILES: CCc1cc(C2CCN(S(C)(=O)=O)CC2)ncc1N(C)c1cc2c(cn1)ncn2C] | (4-Ethyl-1′- ethanesulfonyl- 1′,2′,3′,4′,5′,6′-hexahydro- [2,4′]bipyridinyl-5-yl)- methyl-(1-methyl-1H- imidazo[4,5-c]pyridin-6- yl)-amine | Described above | 429 | 430 |
| 384 | 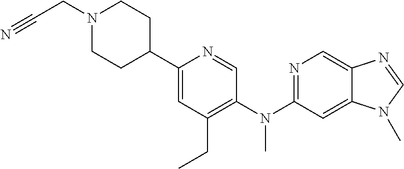 [SMILES: CCc1cc(C2CCN(CC#N)CC2)ncc1N(C)c1cc2c(cn1)ncn2C] | {4-Ethyl-5-[methyl-(1- methyl-1H-imidazo[4,5- c]pyridin-6-yl)-amino]- 3′,4′,5′,6′-tetrahydro-2′H- [2,4′]bipyridinyl-1′-yl}- acetonitrile | Described above | 389 | 390 |
| 385 | 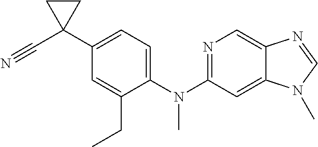 [SMILES: CCc1cc(C2(C#N)CC2)ccc1N(C)c1cc2c(cn1)ncn2C] | 1-{3-Ethyl-4-[methyl-(1- methyl-1H-imidazo[4,5- c]pyridin-6-yl)-amino]- phenyl}- cyclopropanecarbonitrile | Described above | 331 | 332 |
| 386 | 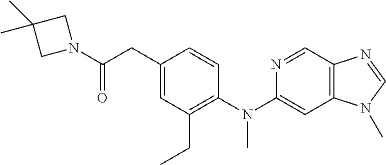 [SMILES: CCc1cc(CC(=O)N2CC(C)(C)C2)ccc1N(C)c1cc2c(cn1)ncn2C] | 1-(3,3-Dimethyl-azetidin- 1-yl)-2-{3-ethyl-4- [methyl-(1-methyl-1H- imidazo[4,5-c]pyridin-6- yl)-amino]-phenyl}- ethanone | Described above | 391 | 392 |
| 387 | 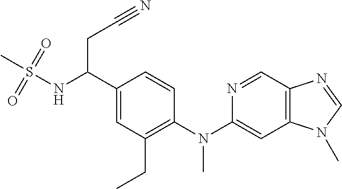 [SMILES: CCc1cc(C(CC#N)NS(C)(=O)=O)ccc1N(C)c1cc2c(cn1)ncn2C] | N-(2-Cyano-1-{3-ethyl-4- [methyl-(1-methyl-1H- imidazo[4,5-c]pyridin-6- yl)-amino]-phenyl}- ethyl)- methanesulfonamide | Described above | 412 | 414 |
| 388 | 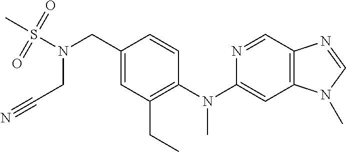 [SMILES: CCc1cc(CN(CC#N)S(C)(=O)=O)ccc1N(C)c1cc2c(cn1)ncn2C] | N-Cyanomethyl-N-{3- ethyl-4-[methyl-(1- methyl-1H-imidazo[4,5- c]pyridin-6-yl)-amino]- benzyl}- methanesulfonamide | Described above | 412 | 413 |
| 389 | 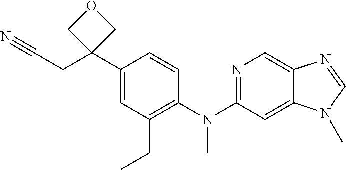 [SMILES: CCc1cc(C2(CC#N)COC2)ccc1N(C)c1cc2c(cn1)ncn2C] | (3-{3-Ethyl-4-[methyl-(1- methyl-1H-imidazo[4,5- c]pyridin-6-yl)-amino]- phenyl}-oxetan-3-yl)- acetonitrile | Described above | 361 | 362 |
| 390 | 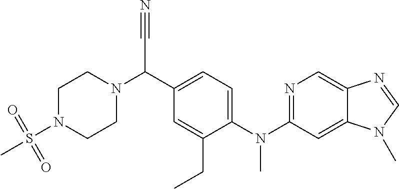 [SMILES: CCc1cc(C(C#N)N2CCN(S(C)(=O)=O)CC2)ccc1N(C)c1cc2c(cn1)ncn2C] | {3-Ethyl-4-[methyl-(1- methyl-1H-imidazo[4,5- c]pyridin-6-yl)-amino]- phenyl}-(4-methane- sulfonyl-piperazin-1-yl)- acetonitrile | Described above | 467.6 | 468.8 |
| 391 | 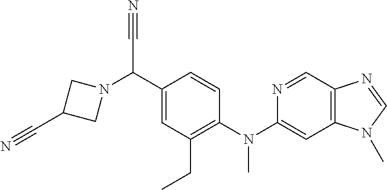 [SMILES: CCc1cc(C(C#N)N2CC(C#N)C2)ccc1N(C)c1cc2c(cn1)ncn2C] | 1-(Cyano-{3-ethyl-4- [methyl-(1-methyl-1H- imidazo[4,5-c]pyridin-6- yl)-amino]-phenyl}- methyl)-azetidine-3- carbonitrile | Described above | 385 | 387 |
| 392 | 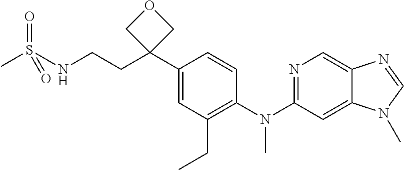 [SMILES: CCc1cc(C2(CCNS(C)(=O)=O)COC2)ccc1N(C)c1cc2c(cn1)ncn2C] | N-[2-(3-{3-Ethyl-4- [methyl-(1-methyl-1H- imidazo[4,5-c]pyridin-6- yl)-amino]-phenyl}- oxetan-3-yl)-ethyl]- methanesulfonamide | Described above | 444 | 444 |
| 393 | 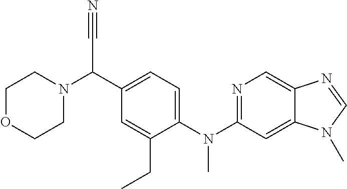 [SMILES: CCc1cc(C(C#N)N2CCOCC2)ccc1N(C)c1cc2c(cn1)ncn2C] | {3-Ethyl-4-[methyl-(1- methyl-1H-imidazo[4,5- c]pyridin-6-yl)-amino]- phenyl}-morpholin-4-yl- acetonitrile | Described above | 390 | 392 |
| 394 | 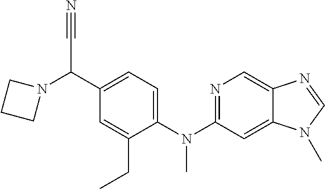 [SMILES: CCc1cc(C(C#N)N2CCC2)ccc1N(C)c1cc2c(cn1)ncn2C] | Azetidin-1-yl-{3-ethyl-4- [methyl-(1-methyl-1H- imidazo[4,5-c]pyridin-6- yl)-amino]-phenyl}- acetonitrile | Described above | 360 | 362 |
| 395 | 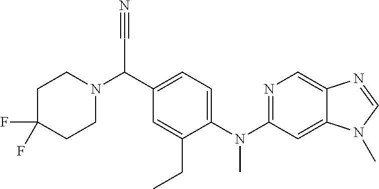 [SMILES: CCc1cc(C(C#N)N2CCC(F)(F)CC2)ccc1N(C)c1cc2c(cn1)ncn2C] | (4,4-Difluoro-piperidin-1- yl)-{3-ethyl-4-[methyl-(1- methyl-1H-imidazo[4,5- c]pyridin-6-yl)-amino]- phenyl}-acetonitrile | Described above | 424 | 426 |
| 396 | 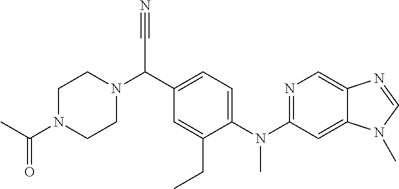 [SMILES: CCc1cc(C(C#N)N2CCN(C(C)=O)CC2)ccc1N(C)c1cc2c(cn1)ncn2C] | (4-Acetyl-piperazin-1-yl)- {3-ethyl-4-[methyl-(1- methyl-1H-imidazo[4,5- c]pyridin-6-yl)-amino]- phenyl}-acetonitrile | Described above | 431 | 433 |
| 397 | 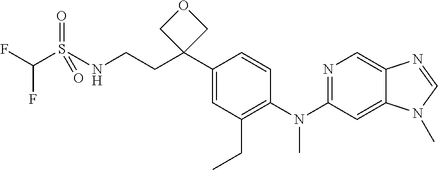 [SMILES: CCc1cc(C2(CCNS(=O)(=O)C(F)F)COC2)ccc1N(C)c1cc2c(cn1)ncn2C] | N-[2-(3-{3-Ethyl-4- [methyl-(1-methyl-1H- imidazo[4,5-c]pyridin-6- yl)-amino]-phenyl}- oxetan-3-yl)-ethyl]-C,C- difluoro- methanesulfonamide | Described above | 479 | 480 |
| 398 | 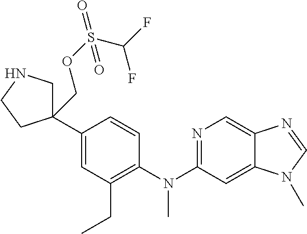 [SMILES: CCc1cc(C2(COS(=O)(=O)C(F)F)CCNC2)ccc1N(C)c1cc2c(cn1)ncn2C] | Difluoro-methanesulfonic acid 3-{3-ethyl-4- [methyl-(1-methyl-1H- imidazo[4,5-c]pyridin-6- yl)-amino]-phenyl}- pyrrolidin-3-ylmethyl ester | Described above | 479 | 480 |
| 399 | 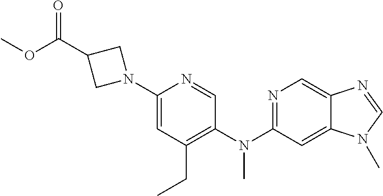 [SMILES: CCc1cc(N2CC(C(=O)OC)C2)ncc1N(C)c1cc2c(cn1)ncn2C] | 1-{4-Ethyl-5-[methyl-(1- methyl-1H-imidazo[4,5- c]pyridin-6-yl)-amino]- pyridin-2-yl}-azetidine-3- carboxylic acid methyl ester | Described above | 380 | 381 |
| 400 | 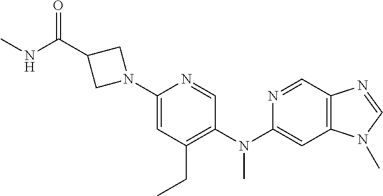 [SMILES: CCc1cc(N2CC(C(=O)NC)C2)ncc1N(C)c1cc2c(cn1)ncn2C] | 1-{4-Ethyl-5-[methyl-(1- methyl-1H-imidazo[4,5- c]pyridin-6-yl)-amino]- pyridin-2-yl}-azetidine-3- carboxylic acid methylamide | Described above | 379 | 380 |
| 401 | 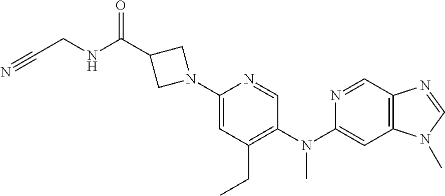 [SMILES: CCc1cc(N2CC(C(=O)NCC#N)C2)ncc1N(C)c1cc2c(cn1)ncn2C] | 1-{4-Ethyl-5-[methyl-(1- methyl-1H-imidazo[4,5- c]pyridin-6-yl)-amino]- pyridin-2-yl}-azetidine-3- carboxylic acid cyanomethyl-amide | Described above | 404 | 405 |
| 402 | 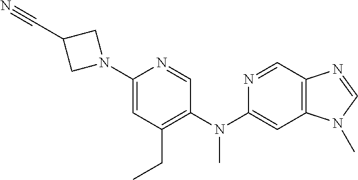 [SMILES: CCc1cc(N2CC(C#N)C2)ncc1N(C)c1cc2c(cn1)ncn2C] | 1-{4-Ethyl-5-[methyl-(1- methyl-1H-imidazo[4,5- c]pyridin-6-yl)-amino]- pyridin-2-yl}-azetidine-3- carbonitrile | Described above | 347 | 348 |
| 403 | 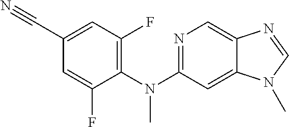 [SMILES: CN(c1cc2c(cn1)ncn2C)c1c(F)cc(C#N)cc1F] | 3,5-Difluoro-4-[methyl- (1-methyl-1H- imidazo[4,5-c]pyridin-6- yl)-amino]-benzonitrile | Described above | 299 | 300 |
| 404 | 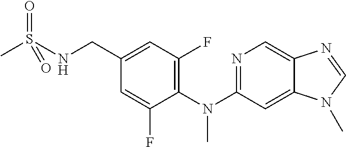 [SMILES: CN(c1cc2c(cn1)ncn2C)c1c(F)cc(CNS(C)(=O)=O)cc1F] | N-{3,5-Difluoro-4- [methyl-(1-methyl-1H- imidazo[4,5-c]pyridin-6- yl)-amino]-benzyl}- methanesulfonamide | Described above | 381 | 382 |
| 405 | 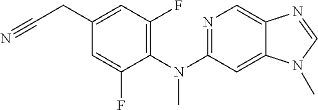 [SMILES: CN(c1cc2c(cn1)ncn2C)c1c(F)cc(CC#N)cc1F] | {3,5-Difluoro-4-[methyl- (1-methyl-1H- imidazo[4,5-c]pyridin-6- yl)-amino]-phenyl}- acetonitrile | Described above | 313 | 314 |
| 406 | 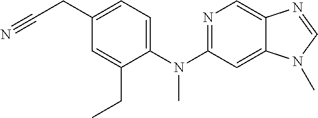 [SMILES: CCc1cc(CC#N)ccc1N(C)c1cc2c(cn1)ncn2C] | {3-Ethyl-4-[methyl-(1- methyl-1H-imidazo[4,5- c]pyridin-6-yl)-amino]- phenyl}-acetonitrile | Described above | 305 | 306 |
| 407 | 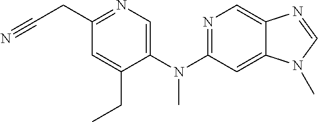 [SMILES: CCc1cc(CC#N)ncc1N(C)c1cc2c(cn1)ncn2C] | {4-Ethyl-5-[methyl-(1- methyl-1H-imidazo[4,5- c]pyridin-6-yl)-amino]- pyridin-2-yl}-acetonitrile | Described above | 306 | 307 |
| 408 | 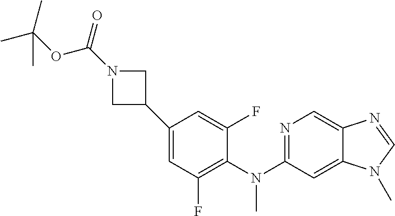 [SMILES: CN(c1cc2c(cn1)ncn2C)c1c(F)cc(C2CN(C(=O)OC(C)(C)C)C2)cc1F] | 3-{3,5-Difluoro-4- [methyl-(1-methyl-1H- imidazo[4,5-c]pyridin-6- yl)-amino]-phenyl}- azetidine-1-carboxylic acid tert-butyl ester | Described above | 429 | 431 |
| 409 | 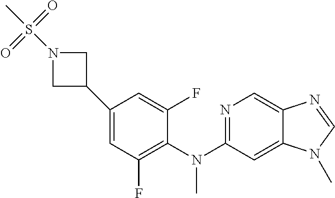 [SMILES: CN(c1cc2c(cn1)ncn2C)c1c(F)cc(C2CN(S(C)(=O)=O)C2)cc1F] | [2,6-Difluoro-4-(1- methanesulfonyl-azetidin- 3-yl)-phenyl]-methyl-(1- methyl-1H-imidazo[4,5- c]pyridin-6-yl)-amine | Described above | 407 | 408 |
| 410 | 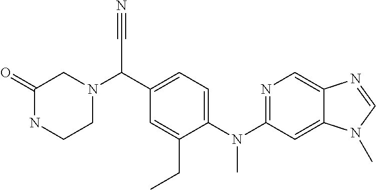 [SMILES: CCc1cc(C(C#N)N2CCNC(=O)C2)ccc1N(C)c1cc2c(cn1)ncn2C] | {3-Ethyl-4-[methyl-(1- methyl-1H-imidazo[4,5- c]pyridin-6-yl)-amino]- phenyl}-(3-oxo-piperazin- 1-yl)-acetonitrile | Described above | 403 | 404 |
| 411 | 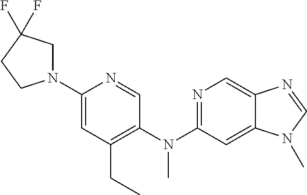 [SMILES: CCc1cc(N2CCC(F)(F)C2)ncc1N(C)c1cc2c(cn1)ncn2C] | [6-(3,3-Difluoro- pyrrolidin-1-yl)-4-ethyl- pyridin-3-yl]-methyl-(1- methyl-1H-imidazo[4,5- c]pyridin-6-yl)-amine | Described above | 372 | 373 |
| 412 | 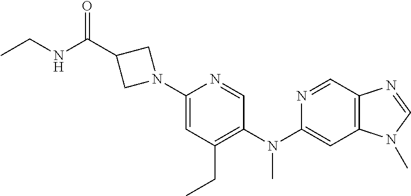 [SMILES: CCNC(=O)C1CN(c2cc(CC)c(N(C)c3cc4c(cn3)ncn4C)cn2)C1] | 1-{4-Ethyl-5-[methyl-(1- methyl-1H-imidazo[4,5- c]pyridin-6-yl)-amino]- pyridin-2-yl}-azetidine-3- carboxylic acid ethylamide | Described above | 393 | 394 |
| 413 | 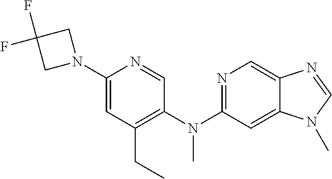 [SMILES: CCc1cc(N2CC(F)(F)C2)ncc1N(C)c1cc2c(cn1)ncn2C] | [6-(3,3-Difluoro-azetidin- 1-yl)-4-ethyl-pyridin-3- yl]-methyl-(1-methyl-1H- imidazo[4,5-c]pyridin-6- yl)-amine | Described above | 358 | 359 |
| 414 | 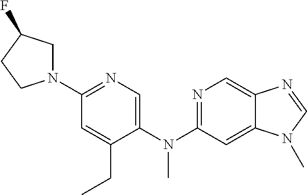 [SMILES: CCc1cc(N2CC[C@@H](F)C2)ncc1N(C)c1cc2c(cn1)ncn2C] | [4-Ethyl-6-((R)-3-fluoro- pyrrolidin-1-yl)-pyridin- 3-yl]-methyl-(1-methyl- 1H-imidazo[4,5- c]pyridin-6-yl)-amine | Described above | 354 | 356 |
| 415 | 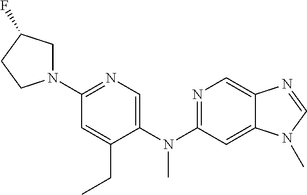 [SMILES: CCc1cc(N2CC[C@H](F)C2)ncc1N(C)c1cc2c(cn1)ncn2C] | [4-Ethyl-6-((S)-3-fluoro- pyrrolidin-1-yl)-pyridin- 3-yl]-methyl-(1-methyl- 1H-imidazo[4,5- c]pyridin-6-yl)-amine | Described above | 354 | 355 |
| 416 | 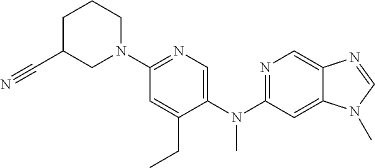 [SMILES: CCc1cc(N2CCCC(C#N)C2)ncc1N(C)c1cc2c(cn1)ncn2C] | 4′-Ethyl-5′-[methyl-(1- methyl-1H-imidazo[4,5- c]pyridin-6-yl)-amino]- 3,4,5,6-tetrahydro-2H- [1,2′]bipyridinyl-3- carbonitrile | Described above | 375 | 376 |
| 417 | 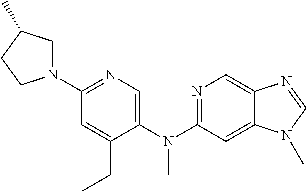 [SMILES: CCc1cc(N2CC[C@H](C)C2)ncc1N(C)c1cc2c(cn1)ncn2C] | [4-Ethyl-6-((S)-3-methyl- pyrrolidin-1-yl)-pyridin- 3-yl]-methyl-(1-methyl- 1H-imidazo[4,5- c]pyridin-6-yl)-amine | Described above | 350 | 351 |
| 418 | 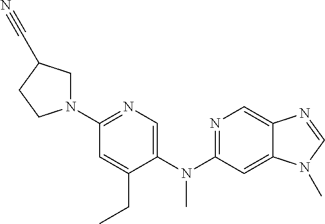 [SMILES: CCc1cc(N2CCC(C#N)C2)ncc1N(C)c1cc2c(cn1)ncn2C] | 1-{4-Ethyl-5-[methyl-(1- methyl-1H-imidazo[4,5- c]pyridin-6-yl)-amino]- pyridin-2-yl}-pyrrolidine- 3-carbonitrile | Described above | 361 | 362 |
| 419 | 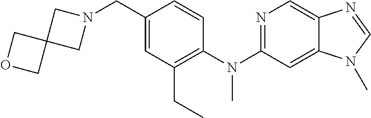 [SMILES: CCc1cc(CN2CC3(COC3)C2)ccc1N(C)c1cc2c(cn1)ncn2C] | [2-Ethyl-4-(2-oxa-6-aza- spiro[3.3]hept-6- ylmethyl)-phenyl]- methyl-(1-methyl-1H- imidazo[4,5-c]pyridin-6- yl)-amine | Described above | 377 | 378 |
| 420 | 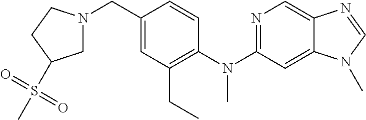 [SMILES: CCc1cc(CN2CCC(S(C)(=O)=O)C2)ccc1N(C)c1cc2c(cn1)ncn2C] | [2-Ethyl-4-(3- methanesulfonyl- pyrrolidin-1-ylmethyl)- phenyl]-methyl-(1- methyl-1H-imidazo[4,5- c]pyridin-6-yl)-amine | Described above | 428 | 428 |
| 421 | 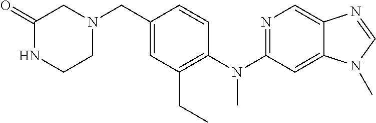 [SMILES: CCc1cc(CN2CCNC(=O)C2)ccc1N(C)c1cc2c(cn1)ncn2C] | 4-{3-Ethyl-4-[methyl-(1- methyl-1H-imidazo[4,5- c]pyridin-6-yl)-amino]- benzyl}-piperazin-2-one | Described above | 378 | 379 |
| 422 | 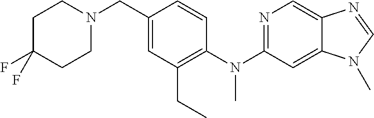 [SMILES: CCc1cc(CN2CCC(F)(F)CC2)ccc1N(C)c1cc2c(cn1)ncn2C] | [4-(4,4-Difluoro- piperidin-1-ylmethyl)-2- ethyl-phenyl]-methyl-(1- methyl-1H-imidazo[4,5- c]pyridin-6-yl)-amine | Described above | 399 | 400 |
| 423 | 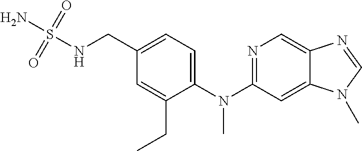 [SMILES: CCc1cc(CNS(N)(=O)=O)ccc1N(C)c1cc2c(cn1)ncn2C] | N-{3-Ethyl-4-[methyl-(1- methyl-1H-imidazo[4,5- c]pyridin-6-yl)-amino]- benzyl}- aminosulfonamide | Described above | 374 | 375 |
| 424 | 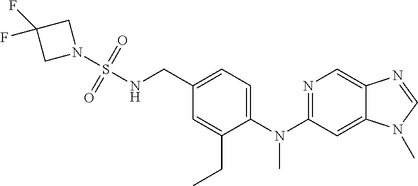 [SMILES: CCc1cc(CNS(=O)(=O)N2CC(F)(F)C2)ccc1N(C)c1cc2c(cn1)ncn2C] | 3,3-Difluoro-azetidine-1- sulfonic acid 3-ethyl-4- [methyl-(1-methyl-1H- imidazo[4,5-c]pyridin-6- yl)-amino]-benzylamide | Described above | 450 | 451 |
| 425 | 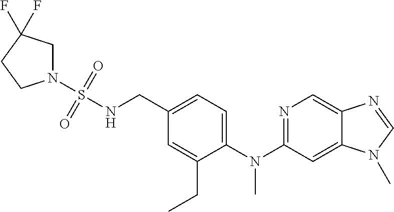 [SMILES: CCc1cc(CNS(=O)(=O)N2CCC(F)(F)C2)ccc1N(C)c1cc2c(cn1)ncn2C] | 3,3-Difluoro-pyrrolidine- 1-sulfonic acid 3-ethyl-4- [methyl-(1-methyl-1H- imidazo[4,5-c]pyridin-6- yl)-amino]-benzylamide | Described above | 464 | 465 |
| 426 | 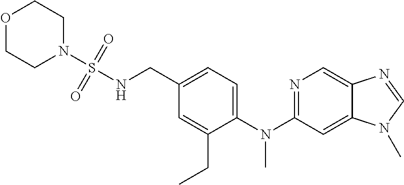 [SMILES: CCc1cc(CNS(=O)(=O)N2CCOCC2)ccc1N(C)c1cc2c(cn1)ncn2C] | Morpholine-4-sulfonic acid 3-ethyl-4-[methyl-(1- methyl-1H-imidazo[4,5- c]pyridin-6-yl)-amino]- benzylamide | Described above | 445 | 445 |
| 427 | 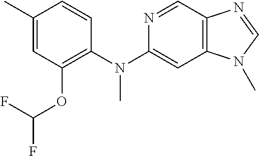 [SMILES: Cc1ccc(N(C)c2cc3c(cn2)ncn3C)c(OC(F)F)c1] | (2-Difluoromethoxy-4- methyl-phenyl)-methyl- (1-methyl-1H- imidazo[4,5-c]pyridin-6- yl)-amine | Described above | 318 | 319 |
| 428 | 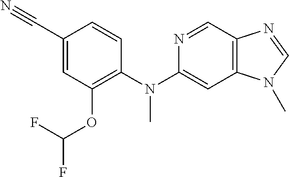 [SMILES: CN(c1cc2c(cn1)ncn2C)c1ccc(C#N)cc1OC(F)F] | 3-Difluoromethoxy-4- [methyl-(1-methyl-1H- imidazo[4,5-c]pyridin-6- yl)-amino]-benzonitrile | Described above | 329 | 331 |
| 429 | 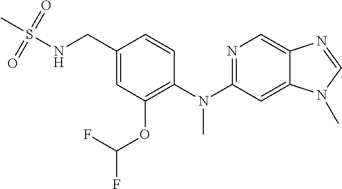 [SMILES: CN(c1cc2c(cn1)ncn2C)c1ccc(CNS(C)(=O)=O)cc1OC(F)F] | N-{3-Difluoromethoxy-4- [methyl-(1-methyl-1H- imidazo[4,5-c]pyridin-6- yl)-amino]-benzyl}- methanesulfonamide | Described above | 411 | 413 |
| 430 | 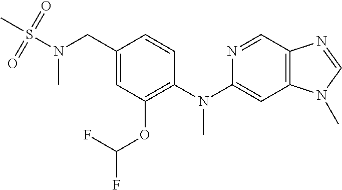 [SMILES: CN(c1cc2c(cn1)ncn2C)c1ccc(CN(C)S(C)(=O)=O)cc1OC(F)F] | N-{3-Difluoromethoxy-4- [methyl-(1-methyl-1H- imidazo[4,5-c]pyridin-6- yl)-amino]-benzyl}-N- methyl- methanesulfonamide | Described above | 425 | 427 |
| 431 | 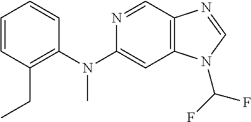 [SMILES: CCc1ccccc1N(C)c1cc2c(cn1)ncn2C(F)F] | (1-Difluoromethyl-1H- imidazo[4,5-c]pyridin-6- yl)-(2-ethyl-phenyl)- methyl-amine | Described above | 302 | 303 |
| 432 | 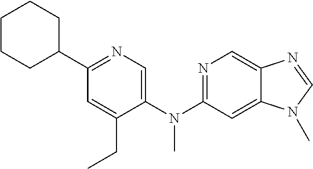 [SMILES: CCc1cc(C2CCCCC2)ncc1N(C)c1cc2c(cn1)ncn2C] | (6-Cyclohexyl-4-ethyl- pyridin-3-yl)-methyl-(1- methyl-1H-imidazo[4,5- c]pyridin-6-yl)-amine | Described above | 349 | 350 |
| 433 | 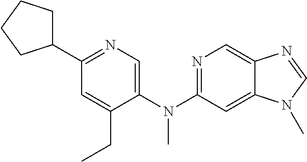 [SMILES: CCc1cc(C2CCCC2)ncc1N(C)c1cc2c(cn1)ncn2C] | (6-Cyclopentyl-4-ethyl- pyridin-3-yl)-methyl-(1- methyl-1H-imidazo[4,5- c]pyridin-6-yl)-amine | Described above | 335 | 336 |
| 434 | 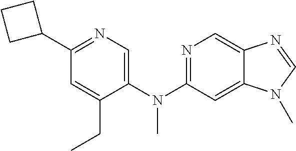 [SMILES: CCc1cc(C2CCC2)ncc1N(C)c1cc2c(cn1)ncn2C] | (6-Cyclobutyl-4-ethyl- pyridin-3-yl)-methyl-(1- methyl-1H-imidazo[4,5- c]pyridin-6-yl)-amine | Described above | 321 | 322 |
| 435 | 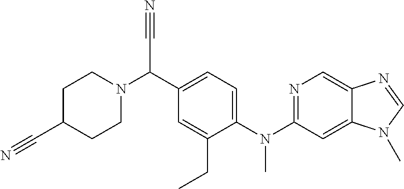 [SMILES: CCc1cc(C(C#N)N2CCC(C#N)CC2)ccc1N(C)c1cc2c(cn1)ncn2C] | 1-(Cyano-{3-ethyl-4- [methyl-(1-methyl-1H- imidazo[4,5-c]pyridin-6- yl)-amino]-phenyl}- methyl)-piperidine-4- carbonitrile | Described above | 413 | 415 |
| 436 | 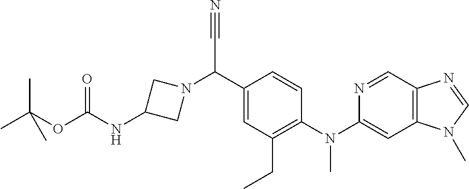 [SMILES: CCc1cc(C(C#N)N2CC(NC(=O)OC(C)(C)C)C2)ccc1N(C)c1cc2c(cn1)ncn2C] | [1-(Cyano-{3-ethyl-4- [methyl-(1-methyl-1H- imidazo[4,5-c]pyridin-6- yl)-amino]-phenyl}- methyl)-azetidin-3-yl]- carbamic acid tert-butyl ester | Described above | 476 | 477 |
| 437 | 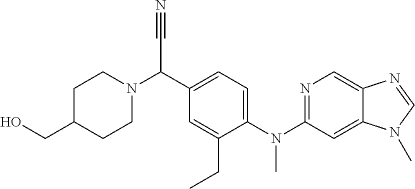 [SMILES: CCc1cc(C(C#N)N2CCC(CO)CC2)ccc1N(C)c1cc2c(cn1)ncn2C] | {3-Ethyl-4-[methyl-(1- methyl-1H-imidazo[4,5- c]pyridin-6-yl)-amino]- phenyl}-(4- hydroxymethyl-piperidin- 1-yl)-acetonitrile | Described above | 418 | 419 |
| 438 | 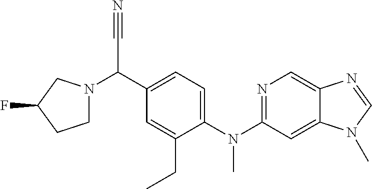 [SMILES: CCc1cc(C(C#N)N2CC[C@@H](F)C2)ccc1N(C)c1cc2c(cn1)ncn2C] | {3-Ethyl-4-[methyl-(1- methyl-1H-imidazo[4,5- c]pyridin-6-yl)-amino]- phenyl}-((R)-3-fluoro- pyrrolidin-1-yl)- acetonitrile | Described above | 392 | 394 |
| 439 | 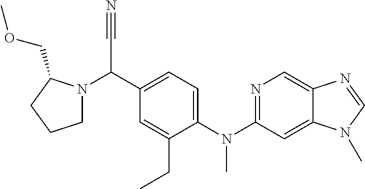 [SMILES: CCc1cc(C(C#N)N2CCC[C@@H]2COC)ccc1N(C)c1cc2c(cn1)ncn2C] | {3-Ethyl-4-[methyl-(1- methyl-1H-imidazo[4,5- c]pyridin-6-yl)-amino]- phenyl}-((R)-2- methoxymethyl- pyrrolidin-1-yl)- acetonitrile | Described above | 418 | 420 |
| 440 | 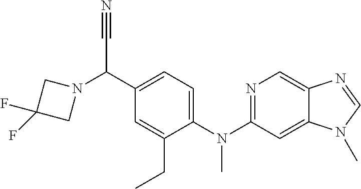 [SMILES: CCc1cc(C(C#N)N2CC(F)(F)C2)ccc1N(C)c1cc2c(cn1)ncn2C] | (3,3-Difluoro-azetidin-1- yl)-{3-ethyl-4-[methyl-(1- methyl-1H-imidazo[4,5- c]pyridin-6-yl)-amino]- phenyl}-acetonitrile | Described above | 396 | 398 |
| 441 | 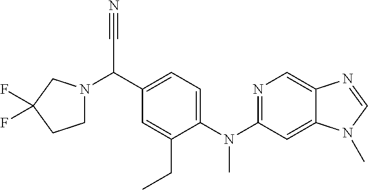 [SMILES: CCc1cc(C(C#N)N2CCC(F)(F)C2)ccc1N(C)c1cc2c(cn1)ncn2C] | (3,3-Difluoro-pyrrolidin- 1-yl)-{3-ethyl-4-[methyl- (1-methyl-1H- imidazo[4,5-c]pyridin-6- yl)-amino]-phenyl}- acetonitrile | Described above | 410 | 411 |
| 442 | 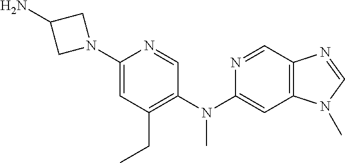 [SMILES: CCc1cc(N2CC(N)C2)ncc1N(C)c1cc2c(cn1)ncn2C] | [6-(3-Amino-azetidin-1- yl)-4-ethyl-pyridin-3-yl]- methyl-(1-methyl-1H- imidazo[4,5-c]pyridin-6- yl)-amine | Described above | 337 | 338 |
| 443 | 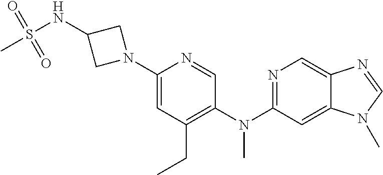 [SMILES: CCc1cc(N2CC(NS(C)(=O)=O)C2)ncc1N(C)c1cc2c(cn1)ncn2C] | N-(1-{4-Ethyl-5-[methyl- (1-methyl-1H- imidazo[4,5-c]pyridin-6- yl)-amino]-pyridin-2-yl}- azetidin-3-yl)- methanesulfonamide | Described above | 415 | 416 |
| 444 | 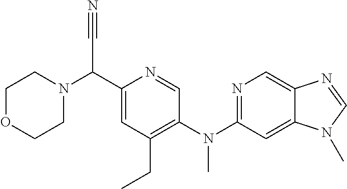 [SMILES: CCc1cc(C(C#N)N2CCOCC2)ncc1N(C)c1cc2c(cn1)ncn2C] | {4-Ethyl-5-[methyl-(1- methyl-1H-imidazo[4,5- c]pyridin-6-yl)-amino]- pyridin-2-yl}-morpholin- 4-yl-acetonitrile | Described above | 391 | 392 |
| 445 | 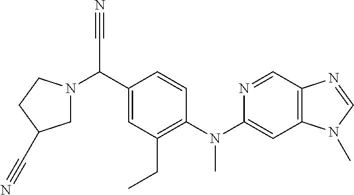 [SMILES: CCc1cc(C(C#N)N2CCC(C#N)C2)ccc1N(C)c1cc2c(cn1)ncn2C] | 1-(Cyano-{3-ethyl-4- [methyl-(1-methyl-1H- imidazo[4,5-c]pyridin-6- yl)-amino]-phenyl}- methyl)-pyrrolidine-3- carbonitrile | Described above | 399 | 400 |
| 446 | 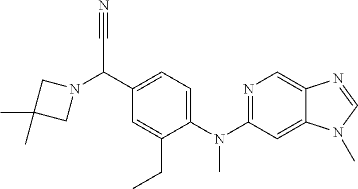 [SMILES: CCc1cc(C(C#N)N2CC(C)(C)C2)ccc1N(C)c1cc2c(cn1)ncn2C] | (3,3-Dimethyl-azetidin-1- yl)-{3-ethyl-4-[methyl-(1- methyl-1H-imidazo[4,5- c]pyridin-6-yl)-amino]- phenyl}-acetonitrile | Described above | 388 | 389 |
| 447 | 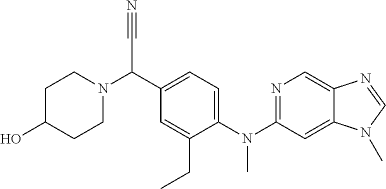 [SMILES: CCc1cc(C(C#N)N2CCC(O)CC2)ccc1N(C)c1cc2c(cn1)ncn2C] | {3-Ethyl-4-[methyl-(1- methyl-1H-imidazo[4,5- c]pyridin-6-yl)-amino]- phenyl}-(4-hydroxy- piperidin-1-yl)- acetonitrile | Described above | 404 | 405 |
| 448 | 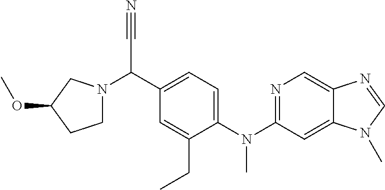 [SMILES: CCc1cc(C(C#N)N2CC[C@@H](OC)C2)ccc1N(C)c1cc2c(cn1)ncn2C] | {3-Ethyl-4-[methyl-(1- methyl-1H-imidazo[4,5- c]pyridin-6-yl)-amino]- phenyl}-((R)-3-methoxy- pyrrolidin-1-yl)- acetonitrile | Described above | 404 | 405 |
| 449 | 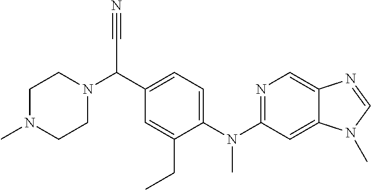 [SMILES: CCc1cc(C(C#N)N2CCN(C)CC2)ccc1N(C)c1cc2c(cn1)ncn2C] | {3-Ethyl-4-[methyl-(1- methyl-1H-imidazo[4,5- c]pyridin-6-yl)-amino]- phenyl}-(4-methyl- piperazin-1-yl)- acetonitrile | Described above | 403 | 404 |
| 450 | 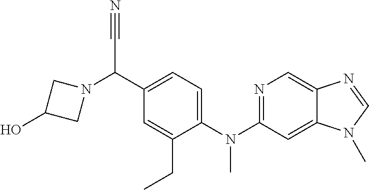 [SMILES: CCc1cc(C(C#N)N2CC(O)C2)ccc1N(C)c1cc2c(cn1)ncn2C] | {3-Ethyl-4-[methyl-(1- methyl-1H-imidazo[4,5- c]pyridin-6-yl)-amino]- phenyl}-(3-hydroxy- azetidin-1-yl)-acetonitrile | Described above | 376 | 377 |
| 451 | 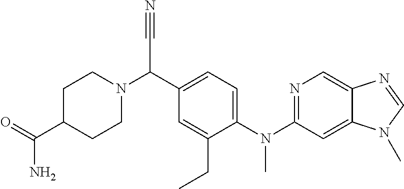 [SMILES: CCc1cc(C(C#N)N2CCC(C(N)=O)CC2)ccc1N(C)c1cc2c(cn1)ncn2C] | 1-(Cyano-{3-ethyl-4- [methyl-(1-methyl-1H- imidazo[4,5-c]pyridin-6- yl)-amino]-phenyl}- methyl)-piperidine-4- carboxylic acid amide | Described above | 431 | 432 |
| 452 | 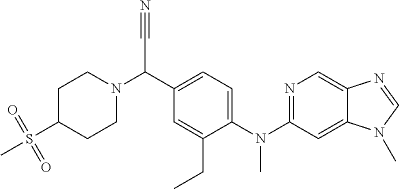 [SMILES: CCc1cc(C(C#N)N2CCC(S(C)(=O)=O)CC2)ccc1N(C)c1cc2c(cn1)ncn2C] | {3-Ethyl-4-[methyl-(1- methyl-1H-imidazo[4,5- c]pyridin-6-yl)-amino]- phenyl}-(4- methanesulfonyl- piperidin-1-yl)- acetonitrile | Described above | 467 | 467 |
| 453 | 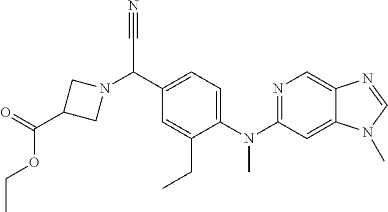 [SMILES: CCOC(=O)C1CN(C(C#N)c2ccc(N(C)c3cc4c(cn3)ncn4C)c(CC)c2)C1] | 1-(Cyano-{3-ethyl-4- [methyl-(1-methyl-1H- imidazo[4,5-c]pyridin-6- yl)-amino]-phenyl}- methyl)-azetidine-3- carboxylic acid ethyl ester | Described above | 432 | 433 |
| 454 | 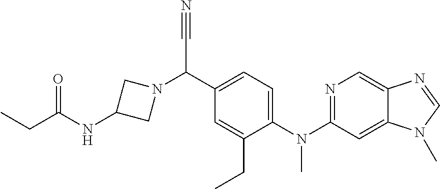 [SMILES: CCC(=O)NC1CN(C(C#N)c2ccc(N(C)c3cc4c(cn3)ncn4C)c(CC)c2)C1] | N-[1-(Cyano-{3-ethyl-4- [methyl-(1-methyl-1H- imidazo[4,5-c]pyridin-6- yl)-amino]-phenyl}- methyl)-azetidin-3-yl]- propionamide | Described above | 431 | 432 |
| 455 | 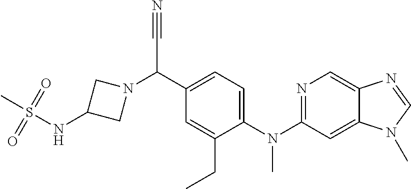 [SMILES: CCc1cc(C(C#N)N2CC(NS(C)(=O)=O)C2)ccc1N(C)c1cc2c(cn1)ncn2C] | N-[1-(Cyano-{3-ethyl-4- [methyl-(1-methyl-1H- imidazo[4,5-c]pyridin-6- yl)-amino]-phenyl}- methyl)-azetidin-3-yl]- methanesulfonamide | Described above | 424 | 424 |
| 456 | 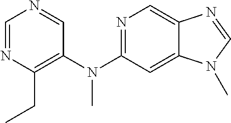 [SMILES: CCc1ncncc1N(C)c1cc2c(cn1)ncn2C] | (4-Ethyl-pyrimidin-5-yl)- methyl-(1-methyl-1H- imidazo[4,5-c]pyridin-6- yl)-amine | Described above | 268 | 269 |
| 457 | 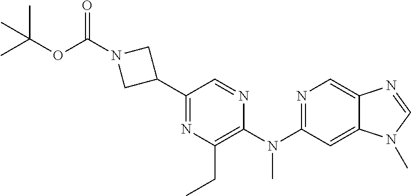 [SMILES: CCc1nc(C2CN(C(=O)OC(C)(C)C)C2)cnc1N(C)c1cc2c(cn1)ncn2C] | 3-{6-Ethyl-5-[methyl-(1- methyl-1H-imidazo[4,5- c]pyridin-6-yl)-amino]- pyrazin-2-yl}-azetidine-1- carboxylic acid tert-butyl ester | Described above | 423 | 424 |
| 458 | 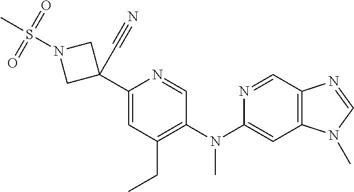 [SMILES: CCc1cc(C2(C#N)CN(S(C)(=O)=O)C2)ncc1N(C)c1cc2c(cn1)ncn2C] | 3-{4-Ethyl-5-[methyl-(1- methyl-1H-imidazo[4,5- c]pyridin-6-yl)-amino]- pyridin-2-yl}-1- methanesulfonyl- azetidine-3-carbonitrile | Described above | 425 | 426 |
| 459 | 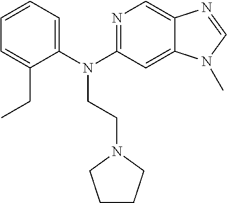 [SMILES: CCc1ccccc1N(CCN1CCCC1)c1cc2c(cn1)ncn2C] | N-(2-ethylphenyl)-1- methyl-N-(2-(pyrrolidin- 1-yl)ethyl)-1H- imidazo[4,5-c]pyridin-6- amine | B | | 350 |
| 460 | 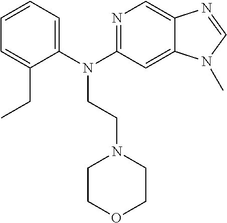 [SMILES: CCc1ccccc1N(CCN1CCOCC1)c1cc2c(cn1)ncn2C] | N-(2-ethylphenyl)-1- methyl-N-(2- morpholinoethyl)-1H- imidazo[4,5-c]pyridin-6- amine | B | 365 | 366 |
| 461 | 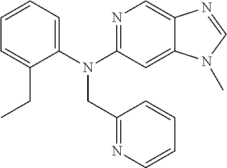 [SMILES: CCc1ccccc1N(Cc1ccccn1)c1cc2c(cn1)ncn2C] | N-(2-ethylphenyl)-1- methyl-N-(pyridin-2- ylmethyl)-1H- imidazo[4,5-c]pyridin-6- amine | B | 343 | 344 |
| 462 | 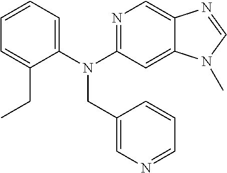 [SMILES: CCc1ccccc1N(Cc1cccnc1)c1cc2c(cn1)ncn2C] | N-(2-ethylphenyl)-1- methyl-N-(pyridin-3- ylmethyl)-1H- imidazo[4,5-c]pyridin-6- amine | B | 343 | 344 |
| 463 | 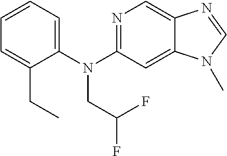 [SMILES: CCc1ccccc1N(CC(F)F)c1cc2c(cn1)ncn2C] | N-(2,2-difluoroethyl)-N- (2-ethylphenyl)-1-methyl- 1H-imidazo[4,5- c]pyridin-6-amine | B | 316 | 317 |
| 464 | 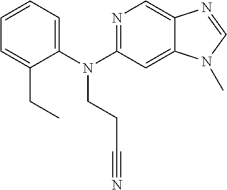 [SMILES: CCc1ccccc1N(CCC#N)c1cc2c(cn1)ncn2C] | 3-((2-ethylphenyl)(1- methyl-1H-imidazo[4,5- c]pyridin-6- yl)amino)propanenitrile | B | 305 | 306 |

Cpd# = Compound number
Mtd: Synthetic method
MW: Calculated molecular weight
MS Ms'd: Measured molecular weight by mass spectrometry

TABLE II
NMR Data of Representative Compounds of the Invention

| Cpd # | (δ) NMR data |
|---|---|
| 1 | ¹H NMR δ (ppm)(DMSO-d₆): 8.60 (1H, d, 2 Hz, Ar); 8.15 (1H, d, 3 Hz, Ar); 7.29 (4H, |
| | m, Ar); 7.01 (1H, d, 2 Hz, Ar); 3.74 (3H, d, 3 Hz, CH₃); 3.44 (3H, d, 3 Hz, CH₃) |
| 26 | ¹H NMR δ (ppm)(CDCl₃): 8.78 (1 H, s, ArH), 7.72 (1 H, s, ArH), 7.22-7.34 (4 H, m, |
| | ArH), 6.61 (1 H, s, ArH), 3.75 (2 H, s, CH₂), 3.68 (3 H, s, CH₃), 3.53 (3 H, s, CH₃). |
| 27 | ¹H NMR δ (ppm)(CDCl₃): 8.74 (1 H, s, ArH), 8.02 (1 H, m, ArH), 7.96 (1 H, m, |
| | ArH), 7.65 (1 H, s, ArH), 7.30 (1 H, d, ArH), 5.99 (1 H, s, ArH), 3.94 (3 H, s, CH₃), |
| | 3.58 (3 H, s, CH₃), 3.48 (3 H, s, CH₃), 2.18 (3 H, s, CH₃). |
| 28 | ¹H NMR δ (ppm)(CDCl₃): 8.72 (1 H, d, ArH), 7.61 (1 H, s, ArH), 7.20-7.13 (1 H, m, |
| | ArH), 7.11-7.06 (1 H, m, ArH), 7.02-6.95 (1 H, m, ArH), 5.85 (1 H, d, ArH), 3.57 (3 |
| | H, s, CH₃), 3.43 (3 H, s, CH₃), 2.57-2.47 (2 H, m, CH₂), 1.21-1.12 (3 H, m, CH₃). |
| 29 | ¹H NMR δ (ppm)(DMSO-d₆): 8.49 (1 H, s, ArH), 7.97 (1 H, s, ArH), 7.29 (1 H, s, |
| | ArH), 7.22 (1 H, d, ArH), 7.15 (1 H, d, ArH), 6.10 (1 H, s, ArH), 5.19 (1 H, t, OH), |
| | 4.52 (2 H, d, CH₂), 3.58 (3 H, s, CH₃), 3.31 (3 H, s, CH₃), 2.07 (3 H, s, CH₃). |
| 30 | ¹H NMR δ (ppm)(CDCl₃): 8.72 (1 H, d, ArH), 7.76 (1 H, d, ArH), 7.66 (1 H, d, ArH), |
| | 7.64 (1 H, s, ArH), 7.27 (1 H, d, ArH), 6.29 (1 H, m, NH), 5.95 (1 H, d, ArH), 3.58 (3 |
| | H, s, CH₃), 3.45 (3 H, s, CH₃), 3.04 (3 H, d, CH₃), 2.17 (3 H, s, CH₃). |
| 31 | ¹H NMR δ (ppm)(DMSO-d₆): 8.49 (1 H, d, ArH), 8.06 (1 H, s, ArH), 7.78 (1 H, d, |
| | ArH), 7.73 (1 H, dd, ArH), 7.40 (1 H, d, ArH), 6.55 (1 H, d, ArH), 3.69 (3 H, s, CH₃), |
| | 3.38 (3 H, s, CH₃), 2.05 (3 H, s, CH₃). |
| 32 | ¹H NMR δ (ppm)(DMSO-d₆): 8.49 (1 H, s, ArH), 8.01 (1 H, s, ArH), 7.94 (1 H, s, |
| | NH), 7.85 (1 H, s, ArH), 7.78 (1 H, dd, ArH), 7.31 (1 H, s, NH), 7.27 (1 H, d, ArH), |
| | 6.28 (1 H, s, ArH), 3.63 (3 H, s, CH₃), 3.36 (3 H, s, CH₃), 2.09 (3 H, s, CH₃). |
| 33 | ¹H NMR δ (ppm)(DMSO-d₆): 8.49 (1 H, d, ArH), 7.96 (1 H, s, ArH), 7.41 (2 H, d, |
| | ArH), 7.11 (1 H, d, ArH), 7.02-6.90 (4 H, m, ArH), 6.04 (1 H, s, ArH), 5.03 (2 H, s, |
| | CH₂), 3.77 (3 H, s, CH₃), 3.57 (3 H, s, CH₃), 3.32 (3 H, d, CH₃), 2.12-1.99 (3 H, m, |
| | CH₃). |
| 34 | ¹H NMR δ (ppm)(DMSO-d₆): 8.50 (1 H, d, ArH), 7.95 (1 H, s, ArH), 7.46-7.42 (1 H, |
| | m, ArH), 7.36-7.29 (2 H, m, ArH), 7.21-7.16 (1 H, m, ArH), 5.91 (1 H, d, ArH), |
| | 3.89 (2 H, d, CH), 3.53 (3 H, s, CH₃), 2.50-2.40 (2 H, m, CH), 1.21-1.05 (6 H, m, CH₃). |
| 35 | ¹H NMR δ (ppm)(DMSO-d₆): 8.43-8.40 (1 H, m, ArH), 7.88-7.81 (1 H, m, ArH), |
| | 7.36-7.31 (1 H, m, ArH), 7.27-7.14 (3 H, m, ArH), 5.78-5.77 (1 H, m, ArH), 3.66 (2 |
| | H, s, CH), 3.42 (3 H, s, CH₃), 2.44-2.35 (2 H, m, CH), 1.10-0.96 (4 H, m, CH), |
| | 0.33-0.25 (2 H, m, CH), 0.06-0.04 (2 H, m, CH). |
| 36 | ¹H NMR δ (ppm)(DMSO-d₆): 8.48 (1 H, d, ArH), 8.06 (1 H, s, ArH), 7.89 (1 H, d, |
| | ArH), 7.82 (1 H, m, ArH), 7.61 (1 H, m, ArH), 7.46 (1 H, d, ArH), 6.38 (1 H, d, |
| | ArH), 3.69 (3 H, s, CH₃), 3.33 (3 H, s, CH₃). |
| 37 | ¹H NMR δ (ppm)(DMSO-d₆): 8.38 (1 H, d, ArH), 8.03 (1 H, s, ArH), 7.33-7.29 (1 H, |
| | m, ArH), 7.25-7.20 (2 H, m, ArH), 7.09-7.05 (1 H, m, ArH), 6.86 (1 H, d, ArH), |
| | 3.74 (3 H, s, CH₃), 3.03-2.97 (1 H, m, CH), 2.41-2.33 (2 H, m, CH), 1.07-1.00 (3 H, m, |
| | CH), 0.92-0.86 (2 H, m, CH), 0.51-0.45 (2 H, m, CH). |
| 38 | ¹H NMR δ (ppm)(DMSO-d₆): 9.38 (1 H, s, OH), 8.49 (1 H, d, ArH), 7.94 (1 H, s, |
| | ArH), 6.98 (1 H, d, ArH), 6.74 (1 H, d, ArH), 6.68 (1 H, dd, ArH), 5.97 (1 H, d, ArH), |
| | 3.55 (3 H, s, CH₃), 3.37-3.24 (3 H, m, CH₃), 1.99 (3 H, s, CH₃). |
| 39 | ¹H NMR δ (ppm)(DMSO-d₆): 8.49 (1 H, d, ArH), 8.06 (1 H, s, ArH), 7.45-7.31 (4 H, |
| | m, ArH), 7.05 (1 H, t, CH), 6.54 (1 H, d, ArH), 3.71 (3 H, s, CH₃), 3.36 (3 H, s, CH₃). |
| 40 | ¹H NMR δ (ppm)(DMSO-d₆): 8.50 (1 H, d, ArH), 8.02 (1 H, s, ArH), 7.89 (1 H, s, |
| | ArH), 7.85 (1 H, d, ArH), 7.29 (1 H, d, ArH), 6.33 (1 H, s, ArH), 3.64 (3 H, s, CH₃), |
| | 3.39 (3 H, s, CH₃), 2.09 (3 H, s CH₃), (OH not observed). |
| 41 | ¹H NMR δ (ppm)(DMSO-d₆): 8.49 (1 H, d, ArH), 8.11 (1 H, s, ArH), 7.82 (1 H, dd, |
| | ArH), 7.73 (1 H, ddd, ArH), 7.45 (1 H, dd, ArH), 7.38-7.33 (1 H, m, ArH), 6.87 (1 H, |
| | d, ArH), 3.75 (3 H, s, CH₃), 3.47 (3 H, s, CH₃). |
| 42 | ¹H NMR δ (ppm)(DMSO-d₆): 8.47 (1 H, d, ArH), 7.97 (1 H, s, ArH), 7.34-7.24 (2 H, |
| | m, ArH), 7.15 (1 H, dd, ArH), 7.04-6.99 (1 H, m, ArH), 6.20 (1 H, d, ArH), 3.71 (3 |
| | H, s, CH₃), 3.59 (3 H, s, CH₃), 3.32 (3 H, s, CH₃). |
| 43 | ¹H NMR δ (ppm)(DMSO-d₆): 8.50 (1 H, d, ArH), 7.98 (1 H, s, ArH), 7.36 (2 H, d, |
| | ArH), 7.24 (1 H, d, ArH), 6.96-6.83 (4 H, m, ArH), 6.12 (1 H, d, ArH), 4.98 (2 H, s, |
| | CH₂), 3.75 (3 H, s, CH₃), 3.59 (3 H, s, CH₃), 3.33 (3 H, s, CH₃), 2.08-1.91 (3 H, m, |
| | CH₃). |
| 44 | ¹H NMR δ (ppm)(DMSO-d₆): 8.50 (1 H, d, ArH), 7.97 (1 H, s, ArH), 7.43 (2 H, d, |
| | ArH), 7.25 (1 H, m, ArH), 7.06-6.91 (3 H, m, ArH), 6.83 (1 H, d, ArH), 6.12 (1 H, d, |
| | ArH), 5.08 (2 H, s, CH₂), 3.77 (3 H, s, CH₃), 3.59 (3 H, s, CH₃), 3.33-3.31 (3 H, m, |
| | CH₃), 1.94 (3 H, s, CH₃). |
| 45 | ¹H NMR δ (ppm)(DMSO-d₆): 8.49 (1 H, d, ArH), 8.02-7.86 (1 H, m, ArH), 7.10 (1 H, |
| | m, ArH), 6.93 (1 H, d, ArH), 6.86 (1 H, dd, ArH), 6.03 (1 H, d, ArH), 3.88-3.68 (3 H, |
| | m, CH₃), 3.57 (3 H, s, CH₃), 3.35-3.28 (3 H, m, CH₃), 2.15-1.98 (3 H, m, CH₃). |
| 46 | ¹H NMR δ (ppm)(DMSO-d₆): 9.46 (1 H, s, OH), 8.50 (1 H, d, ArH), 7.96 (1 H, s, |
| | ArH), 7.08 (1 H, m, ArH), 6.79 (1 H, d, ArH), 6.66 (1 H, d, ArH), 6.07 (1 H, d, ArH), |
| | 3.57 (3 H, s, CH₃), 3.39-3.25 (3 H, m, CH₃), 1.89 (3 H, s, CH₃). |
| 47 | ¹H NMR δ (ppm)(DMSO-d₆): 8.49 (1 H, d, ArH), 7.98 (1 H, s, ArH), 7.20 (1 H, d, |
| | ArH), 7.06 (1 H, d, ArH), 6.99 (1 H, dd, ArH), 6.13 (1 H, d, ArH), 5.19 (2 H, s, CH₂), |
| | 3.60 (3 H, s, CH₃), 3.32 (3 H, d, CH₃), 2.13-2.02 (3 H, m, CH₃). |
| 48 | ¹H NMR δ (ppm)(DMSO-d₆): 8.49 (1 H, d, ArH), 7.96 (1 H, s, ArH), 7.12 (1 H, d, |
| | ArH), 6.95 (1 H, d, ArH), 6.87 (1 H, dd, ArH), 6.04 (1 H, d, ArH), 4.06 (2 H, t, CH₂), |
| | 3.57 (3 H, s, CH₃), 3.32 (3 H, s, CH₃), 2.68 (2 H, t, CH₂), 2.09-2.00 (5 H, m, CH₂, |
| | CH₃). |
| 49 | ¹H NMR δ (ppm)(DMSO-d₆): 8.49 (1 H, d, ArH), 8.08 (1 H, s, ArH), 7.54-7.38 (4 H, |
| | m, ArH), 6.60 (1 H, s, ArH), 3.72 (3 H, s, CH₃), 3.36 (3 H, s, CH₃). |
| 50 | ¹H NMR δ (ppm)(CHCl₃): 8.73 (1 H, s, ArH), 7.77 (1 H, d, ArH), 7.67 (1 H, d, ArH), |
| | 7.64 (1 H, s, ArH), 7.29 (1 H, d, ArH), 6.54 (1 H, m, NH), 5.95 (1 H, d, ArH), |
| | 3.72-3.65 (2 H, m, CH₂), 3.62-3.56 (2 H, m, CH₂), 3.58 (3 H, s, CH₃), 3.46 (3 H, s, CH₃), |
| | 3.41 (3 H, m, CH₃), 2.19 (3 H, s, CH₃). |
| 51 | ¹H NMR δ (ppm)(DMSO-d₆): 8.49 (1 H, d, ArH), 7.96 (1 H, s, ArH), 7.10 (1 H, d, |
| | ArH), 6.91 (1 H, s, ArH), 6.82 (1 H, dd, ArH), 6.07 (1 H, s, ArH), 4.81 (2 H, s, CH₂), |
| | 3.58 (3 H, s, CH₃), 3.31 (3 H, s, CH₃), 3.02 (3 H, s, CH₃), 2.87 (3 H, s, CH₃), |
| | 2.11-2.00 (3 H, m, CH₃). |
| 52 | ¹H NMR δ (ppm)(DMSO-d₆): 8.49 (1 H, d, ArH), 7.96 (1 H, s, ArH), 7.10 (1 H, d, |
| | ArH), 6.93 (1 H, d, ArH), 6.85 (1 H, dd, ArH), 6.03 (1 H, s, ArH), 4.08 (2 H, t, CH₂), |
| | 3.57 (3 H, s, CH₃), 3.34-3.28 (7 H, m, CH, CH₃), 2.80 (2 H, t, CH₂), 2.10-2.02 (3 H, |
| | m, CH₃), 1.72-1.67 (4 H, m, CH). |
| 53 | ¹H NMR δ (ppm)(DMSO-d₆): 8.49 (1 H, d, ArH), 7.96 (1 H, s, ArH), 7.51-7.31 (5 H, |
| | m, ArH), 7.12 (1 H, d, ArH), 7.03 (1 H, d, ArH), 6.94 (1 H, dd, ArH), 6.05 (1 H, s, |
| | ArH), 5.12 (2 H, s, CH₂), 3.58 (3 H, s, CH₃), 3.35-3.28 (3 H, m, CH₃), 2.15-1.98 (3 H, |
| | m, CH₃). |
| 54 | ¹H NMR δ (ppm)(DMSO-d₆): 8.49 (1 H, d, ArH), 7.96 (1 H, s, ArH), 7.54-7.45 (4 H, |
| | m, ArH), 7.12 (1 H, d, ArH), 7.02 (1 H, d, ArH), 6.93 (1 H, dd, ArH), 6.05 (1 H, s, |
| | ArH), 5.13 (2 H, s, CH₂), 3.58 (3 H, s, CH₃), 3.36-3.28 (3 H, m, CH₃), 2.12-2.00 (3 H, |
| | m, CH₃). |
| 55 | ¹H NMR δ (ppm)(DMSO-d₆): 8.71 (1 H, d, ArH), 8.57 (1 H, dd, ArH), 8.49 (1 H, d, |
| | ArH), 7.96 (1 H, s, ArH), 7.91 (1 H, m, ArH), 7.46 (1 H, dd, ArH), 7.14 (1 H, d, |
| | ArH), 7.08-7.03 (1 H, m, ArH), 6.96 (1 H, dd, ArH), 6.05 (1 H, s, ArH), 5.18 (2 H, s, |
| | CH₂), 3.58 (3 H, s, CH₃), 3.32 (3 H, d, CH₃), 2.13-2.00 (3 H, m, CH₃). |
| 56 | ¹H NMR δ (ppm)(DMSO-d₆): 8.50 (1 H, d, ArH), 7.98 (1 H, s, ArH), 7.47-7.28 (5 H, |
| | m, ArH), 7.25 (1 H, d, ArH), 6.93-6.86 (2 H, m, ArH), 6.13 (1 H, d, ArH), 5.07 (2 H, |
| | s, CH₂), 3.59 (3 H, s, CH₃), 3.33 (3 H, s, CH₃), 1.98 (3 H, s, CH₃). |
| 57 | ¹H NMR δ (ppm)(DMSO-d₆): 8.51 (1 H, d, ArH), 7.99 (1 H, s, ArH), 7.36-7.30 (1 H, |
| | m, ArH), 6.98-6.94 (2 H, m, ArH), 6.21 (1 H, d, ArH), 5.15 (2 H, s, CH₂), 3.61 (3 H, |
| | s, CH₃), 3.39-3.28 (3 H, m, CH₃), 2.02 (3 H, s, CH₃). |
| 58 | ¹H NMR δ (ppm)(DMSO-d₆): 8.50 (1 H, d, ArH), 7.98 (1 H, s, ArH), 7.23 (1 H, d, |
| | ArH), 6.83 (1 H, dd, ArH), 6.77 (1 H, d, ArH), 6.13 (1 H, d, ArH), 4.03 (2 H, t, CH₂), |
| | 3.66-3.56 (3 H, m, CH, CH₃), 3.36-3.32 (7 H, s, CH₃), 2.76 (2 H, t, CH₂), 2.04-1.94 (3 |
| | H, m, CH₃), 1.69-1.62 (4 H, m, CH). |
| 59 | ¹H NMR δ (ppm)(DMSO-d₆): 8.50 (1 H, d, ArH), 7.98 (1 H, s, ArH), 7.23 (1 H, d, |
| | ArH), 6.82-6.77 (2 H, m, ArH), 6.14 (1 H, d, ArH), 4.76 (2 H, s, CH₂), 3.61 (3 H, s, |
| | CH₃), 3.33 (3 H, d, CH₃), 3.13-2.85 (3 H, m, CH₃), 2.89-2.76 (3 H, m, CH₃), |
| | 2.07-1.93 (3 H, m, CH₃). |
| 60 | ¹H NMR δ (ppm)(DMSO-d₆): 8.50 (1 H, d, ArH), 7.98 (1 H, s, ArH), 7.25 (1 H, d, |
| | ArH), 6.87-6.78 (2 H, m, ArH), 6.15 (1 H, d, ArH), 4.01 (2 H, t, CH₂), 3.60 (3 H, s, |
| | CH₃), 3.36-3.31 (3 H, m, CH₃), 2.64 (2 H, t, CH₂), 2.06-1.96 (5 H, m, CH₂, CH₃). |
| 61 | ¹H NMR δ (ppm)(DMSO-d₆): 8.66 (1 H, d, ArH), 8.55-8.50 (2 H, m, ArH), 7.99 (1 H, |
| | s, ArH), 7.87 (1 H, m, ArH), 7.42 (1 H, dd, ArH), 7.26 (1 H, d, ArH), 6.95-6.89 (2 H, |
| | m, ArH), 6.13 (1 H, d, ArH), 5.13 (2 H, s, CH₂), 3.60 (3 H, s, CH₃), 3.33 (3 H, s, |
| | CH₃), 1.99 (3 H, s, CH₃). |
| 62 | ¹H NMR δ (ppm)(DMSO-d₆): 8.50 (1 H, d, ArH), 7.98 (1 H, s, ArH), 7.49-7.40 (4 H, |
| | m, ArH), 7.25 (1 H, d, ArH), 6.92-6.85 (2 H, m, ArH), 6.12 (1 H, d, ArH), 5.08 (2 H, |
| | s, CH₂), 3.59 (3 H, s, CH₃), 3.32 (3 H, s, CH₃), 1.98 (3 H, s, CH₃). |
| 63 | ¹H NMR δ (ppm)(DMSO-d₆): 8.50 (1 H, d, ArH), 7.98 (1 H, s, ArH), 7.23 (1 H, d, |
| | ArH), 6.82 (1 H, dd, ArH), 6.76 (1 H, d, ArH), 6.13 (1 H, d, ArH), 3.71 (2 H, d, CH₂), |
| | 3.60 (3 H, s, CH₃), 3.33 (3 H, s, CH₃), 2.04-1.93 (4 H, m, CH, CH₃), 0.97 (6 H, d, |
| | CH₃). |
| 64 | ¹H NMR δ (ppm)(DMSO-d₆): 8.49 (1 H, d, ArH), 7.99 (1 H, s, ArH), 7.33 (1 H, dd, |
| | ArH), 7.08 (1 H, d, ArH), 6.96 (1 H, d, ArH), 6.20 (1 H, d, ArH), 5.23 (2 H, s, CH₂), |
| | 3.61 (3 H, s, CH₃), 3.33 (3 H, s, CH₃), 1.95 (3 H, s, CH₃). |
| 65 | ¹H NMR δ (ppm)(DMSO-d₆): 8.50 (1 H, d, ArH), 8.03-7.95 (1 H, m, ArH), 7.21 (1 H, |
| | dd, ArH), 6.83 (2 H, dd, ArH), 6.15 (1 H, s, ArH), 4.87 (2 H, s, CH₂), 3.60 (3 H, s, |
| | CH₃), 3.33 (3 H, s, CH₃), 3.03 (3 H, s, CH₃), 2.87 (3 H, s, CH₃), 1.97 (3 H, s, CH₃). |
| 66 | ¹H NMR δ (ppm)(DMSO-d₆): 8.49 (1 H, s, ArH), 7.97 (1 H, s, ArH), 7.25 (1 H, dd, |
| | ArH), 6.94 (1 H, d, ArH), 6.83 (1 H, d, ArH), 6.13 (1 H, s, ArH), 4.10 (2 H, t, CH₂), |
| | 3.59 (3 H, s, CH₃), 3.32 (3 H, s, CH₃), 2.70 (2 H, t, CH₂), 2.08 (2 H, m, CH), 1.95 (3 |
| | H, s, CH₃). |
| 67 | ¹H NMR δ (ppm)(DMSO-d₆): 8.48 (1 H, d, ArH), 8.05 (1 H, s, ArH), 7.45-7.37 (1 H, |
| | m, ArH), 7.31-7.22 (3 H, m, ArH), 6.58 (1 H, s, ArH), 3.69 (3 H, s, CH₃), 3.39 (3 H, |
| | s, CH₃). |
| 68 | ¹H NMR δ (ppm)(DMSO-d₆): 8.47 (1 H, s, ArH), 8.02 (1 H, s, ArH), 7.59 (1 H, d, |
| | ArH), 7.47-7.38 (2 H, m, ArH), 7.39-7.31 (1 H, m, ArH), 6.39 (1 H, s, ArH), 3.66 (3 |
| | H, s, CH₃), 3.35 (3 H, s, CH₃). |
| 69 | ¹H NMR δ (ppm)(DMSO-d₆): 8.72 (1 H, d, ArH), 8.56 (1 H, dd, ArH), 8.50 (1 H, d, |
| | ArH), 7.98 (1 H, s, ArH), 7.92 (1 H, m, ArH), 7.46 (1 H, dd, ArH), 7.28 (1 H, dd, |
| | ArH), 7.07 (1 H, d, ArH), 6.86 (1 H, d, ArH), 6.14 (1 H, s, ArH), 5.22 (2 H, s, CH₂), |
| | 3.59 (3 H, s, CH₃), 3.32 (3 H, s, CH₃), 1.96 (3 H, s, CH₃). |
| 70 | ¹H NMR δ (ppm)(DMSO-d₆): 8.49 (1 H, d, ArH), 7.97 (1 H, s, ArH), 7.23 (1 H, dd, |
| | ArH), 6.91 (1 H, d, ArH), 6.80 (1 H, d, ArH), 6.14 (1 H, d, ArH), 3.80 (2 H, d, CH₂), |
| | 3.59 (3 H, s, CH₃), 3.33 (3 H, s, CH₃), 2.07 (1 H, m, CH), 1.95 (3 H, s, CH₃), 1.03 (6 |
| | H, d, CH₃). |
| 71 | ¹H NMR δ (ppm)(CHCl₃): 8.73 (1 H, d, ArH), 7.89 (2 H, s, ArH), 7.66 (1 H, s, NH), |
| | 717.49-7.42 (2 H, m, ArH), 7.23 (2 H, d, ArH), 5.99 (1 H, d, ArH), 3.59 (3 H, s, CH₃), |
| | 3.47 (3 H, s, CH₃), 2.19 (3 H, s, CH₃). |
| 72 | ¹H NMR δ (ppm)(CHCl₃): 9.22 (1 H, s, ArH), 9.01 (2 H, s, ArH), 8.74 (1 H, s, ArH), |
| | 7.70 (1 H, s, ArH), 7.57-7.48 (2 H, m, ArH), 7.39 (1 H, d, ArH), 6.06 (1 H, d, ArH), |
| | 3.64 (3 H, s, CH₃), 3.49 (3 H, s, CH₃), 2.25 (3 H, s, CH₃). |
| 73 | ¹H NMR δ (ppm)(CHCl₃): 8.73 (1 H, d, ArH), 7.79 (1 H, s, ArH), 7.63 (2 H, d, ArH), |
| | 7.25 (1H, s, ArH), 7.39 (1 H, m, ArH), 7.21 (1 H, d, ArH), 5.95 (1 H, d, ArH), |
| | 3.97 (3 H, s, CH₃), 3.57 (3 H, s, CH₃), 3.45 (3 H, m, CH₃), 2.17 (3 H, s, CH₃). |
| 74 | ¹H NMR δ (ppm)(CHCl₃): 8.71 (1 H, s, ArH), 7.60 (1 H, s, ArH), 7.20-7.05 (5 H, m, |
| | ArH), 6.87 (2 H, d, ArH), 5.89 (1 H, s, ArH), 3.94 (2 H, s, CH₂), 3.80 (3 H, s, CH₃), |
| | 3.56 (3 H, s, CH₃), 3.42 (3 H, s, CH₃), 2.10 (3 H, s, CH₃). |
| 75 | ¹H NMR δ (ppm)(DMSO-d₆): 8.48 (1 H, d, ArH), 7.96 (1 H, s, ArH), 7.49 (2 H, d, |
| | ArH), 7.45-7.37 (2 H, m, ArH), 7.37-7.29 (1 H, m, ArH), 7.24 (1 H, t, ArH), 7.03 (1 |
| | H, d, ArH), 6.82 (1 H, d, ArH), 6.12 (1 H, d, ArH), 5.15 (2 H, s, CH₂), 3.57 (3 H, s, |
| | CH₃), 3.31 (3 H, s, CH₃), 1.96 (3 H, s, CH₃). |
| 76 | ¹H NMR δ (ppm)(DMSO-d₆): 8.48 (1 H, d, ArH), 7.96 (1 H, s, ArH), 7.53-7.42 (4 H, |
| | m, ArH), 7.24 (1 H, m, ArH), 7.01 (1 H, d, ArH), 6.83 (1 H, d, ArH), 6.12 (1 H, s, |
| | ArH), 5.15 (2 H, s, CH₂), 3.57 (3 H, s, CH₃), 3.31 (3 H, s, CH₃), 1.96 (3 H, s, CH₃). |
| 77 | ¹H NMR δ (ppm)(CHCl₃): 8.73 (1 H, s, ArH), 7.60 (1 H, s, ArH), 7.27 (1 H, s, ArH), |
| | 6.85 (2 H, m, ArH), 5.90 (1 H, s, ArH), 4.18 (2 H, t, CH₂), 3.56 (3 H, s, CH₃), 3.43 (3 |
| | H, s, CH₃), 2.97 (2 H, t, CH₂), 2.68 (4 H, s, CH), 2.05 (2 H, s, CH), 1.82 (3 H, s, |
| | CH₃), 1.74 (2 H, s, CH). |
| 78 | ¹H NMR δ (ppm)(DMSO-d₆): 8.48-8.41 (2 H, m, ArH), 8.36 (1 H, s, ArH), 8.02 (1 H, |
| | s, ArH), 7.40 (1 H, d, ArH), 6.39 (1 H, d, ArH), 3.67 (3 H, s, CH₃), 3.37 (3 H, s, |
| | CH₃), 2.49-2.39 (2 H, m, CH₂), 1.14-1.05 (3 H, m, CH₃). |
| 79 | ¹H NMR δ (ppm)(DMSO-d₆): 8.47 (2 H, s, ArH), 8.02 (1 H, s, ArH), 7.61 (1 H, d, |
| | ArH), 7.35-7.30 (1 H, m, ArH), 6.36 (1 H, s, ArH), 3.66 (3 H, s, CH₃), 3.35 (3 H, s, |
| | CH₃), 2.60-2.51 (2 H, m, CH₂), 1.17-1.09 (3 H, m, CH₃). |
| 80 | ¹H NMR δ (ppm)(CHCl₃): 8.73 (1 H, s, ArH), 8.08 (1 H, s, ArH), 7.97 (1 H, dd, |
| | ArH), 7.64 (1 H, s, ArH), 7.27 (1 H, d, ArH), 5.93 (1 H, d, ArH), 3.95 (3 H, s, CH₃), |
| | 3.57 (3 H, s, CH₃), 3.47 (3 H, s, CH₃), 2.56 (2 H, q, CH₂), 1.19 (3 H, t, CH₃). |
| 81 | ¹H NMR δ (ppm)(CHCl₃): 8.70 (1 H, d, ArH), 7.61 (1 H, s, ArH), 7.41 (1 H, d, ArH), |
| | 7.30 (1 H, m, ArH), 7.20 (1 H, d, ArH), 5.90 (1 H, d, ArH), 4.76 (2 H, s, CH₂), |
| | 3.56 (3 H, s, CH₃), 3.44 (3 H, s, CH₃), 2.54 (2 H, q, CH₂), 2.25 (1 H, s, OH), 1.16 (3 H, t, |
| | CH₃). |
| 82 | ¹H NMR δ (ppm)(DMSO-d₆): 8.47 (1 H, d, ArH), 8.05 (1 H, s, ArH), 7.84-7.81 (1 H, |
| | m, ArH), 7.74 (1 H, dd, ArH), 7.38 (1 H, d, ArH), 6.51 (1 H, d, ArH), 3.69 (3 H, s, |
| | CH₃), 3.36 (3 H, s, CH₃), 2.44 (2 H, q, CH₂), 1.05 (3 H, t, CH₃). |
| 83 | ¹H NMR δ (ppm)(DMSO-d₆): 8.46 (1 H, d, ArH), 8.03 (1 H, s, ArH), 7.74-7.69 (2 H, |
| | m, ArH), 7.57 (1 H, d, ArH), 6.43 (1 H, d, ArH), 3.68 (3 H, s, CH₃), 3.33 (3 H, s, |
| | CH₃), 2.51-2.45 (2 H, m, CH₂), 1.09 (3 H, t, CH₃). |
| 84 | ¹H NMR δ (ppm)(DMSO-d₆): 8.49 (1 H, d, ArH), 8.01 (1 H, s, ArH), 7.43-7.36 (1 H, |
| | m, ArH), 7.16-7.03 (2 H, m, ArH), 6.27 (1 H, d, ArH), 3.63 (3 H, s, CH₃), 3.34 (3 H, |
| | s, CH₃), 2.45-2.35 (2 H, m, CH₂), 1.11-1.02 (3 H, m, CH₃). |
| 85 | ¹H NMR δ (ppm)(DMSO-d₆): 8.48 (1 H, s, ArH), 8.44 (1 H, d, ArH), 8.00 (1 H, s, |
| | ArH), 7.85 (1 H, d, ArH), 7.75 (1 H, dd, ArH), 7.26 (1 H, d, ArH), 6.24 (1 H, m, NH), |
| | 3.62 (3 H, s, CH₃), 3.35 (3 H, s, CH₃), 2.81 (3 H, d, CH₃), 2.50 (2 H, m, CH₂), 1.12 (3 |
| | H, t, CH₃). |
| 86 | ¹H NMR δ (ppm)(CHCl₃): 8.71 (1 H, d, ArH), 7.85 (1 H, d, ArH), 7.76-7.64 (3 H, m, |
| | ArH), 6.97 (1 H, t, NH), 5.94 (1 H, d, ArH), 3.75 (2 H, m, CH₂), 3.59 (3 H, s, CH₃), |
| | 3.45 (3 H, s, CH₃), 2.80 (2 H, t, CH₂), 2.55 (2 H, q, CH₂), 1.17 (3 H, t, CH₃). |
| 87 | ¹H NMR δ (ppm)(CHCl₃-d): 8.72 (1 H, d, ArH), 7.63 (1 H, s, ArH), 7.46-7.37 (2 H, |
| | m, ArH), 7.29 (1 H, dd, ArH), 5.94 (1 H, d, ArH), 3.57 (3 H, s, CH₃), 3.46 (3 H, s, |
| | CH₃), 3.07 (6 H, m, CH₃), 2.56 (2 H, q, CH₂), 1.17 (3 H, t, CH₃). |
| 88 | ¹H NMR δ (ppm)(DMSO-d₆): 8.61-8.59 (1 H, m, ArH), 8.51 (1 H, d, ArH), 8.08 (1 |
| | H, dd, ArH), 8.00 (1 H, s, ArH), 7.63-7.57 (1 H, m, ArH), 7.31 (1 H, d, ArH), 7.22 (1 |
| | H, d, ArH), 7.13 (1 H, dd, ArH), 6.26 (1 H, d, ArH), 3.65 (3 H, s, CH₃), 3.36 (3 H, m, |
| | CH₃), 2.17-2.05 (3 H, m, CH₃). |
| 89 | ¹H NMR δ (ppm)(DMSO-d₆): 8.88 (1 H, d, ArH), 8.49 (1 H, d, ArH), 8.17-8.09 (2 H, |
| | m, ArH), 7.97 (1 H, s, ArH), 7.15 (1 H, d, ArH), 7.06 (1 H, d, ArH), 6.97 (1 H, dd, |
| | ArH), 6.06 (1 H, d, ArH), 5.31 (2 H, s, CH₂), 3.58 (3 H, s, CH₃), 3.35-3.29 (3 H, m, |
| | CH₃), 2.14-1.99 (3 H, m, CH₃). |
| 90 | ¹H NMR δ (ppm)(DMSO-d₆): 8.55-8.50 (2 H, m, ArH), 8.10-7.98 (2 H, m, ArH), |
| | 7.48-7.39 (2 H, m, ArH), 7.25 (1 H, d, ArH), 7.13 (1 H, d, ArH), 6.35 (1 H, d, ArH), |
| | 3.66 (3 H, s, CH₃), 3.41 (3 H, s, CH₃), 1.85 (3 H, s, CH₃). |
| 91 | ¹H NMR δ (ppm)(DMSO-d₆): 8.55-8.49 (2 H, m, ArH), 8.07-7.99 (2 H, m, ArH), |
| | 7.56 (1 H, dd, ArH), 7.48-7.42 (1 H, m, ArH), 7.12-7.08 (2 H, m, ArH), 6.36 (1 H, d, |
| | ArH), 3.66 (3 H, s, CH₃), 3.39-3.29 (3 H, m, CH₃), 2.11-2.07 (3 H, m, CH₃). |
| 92 | ¹H NMR δ (ppm)(DMSO-d₆): 8.83 (1 H, d, ArH), 8.50 (1 H, d, ArH), 8.13-8.05 (2 H, |
| | m, ArH), 7.99 (1 H, s, ArH), 7.28 (1 H, d, ArH), 6.96-6.90 (2 H, m, ArH), 6.14 (1 H, |
| | d, ArH), 5.26 (2 H, s, CH₂), 3.60 (3 H, s, CH₃), 3.32 (3 H, s, CH₃), 1.99 (3 H, s, CH₃). |
| 93 | ¹H NMR δ (ppm)(DMSO-d₆): 8.49 (1 H, s, ArH), 8.17 (1 H, s, ArH), 7.97 (1 H, s, |
| | ArH), 7.89 (1 H, s, ArH), 7.58-7.56 (1 H, m, ArH), 7.50-7.46 (1 H, m, ArH), |
| | 7.18-7.12 (1 H, m, ArH), 6.14 (1 H, s, ArH), 3.88 (3 H, s, CH₃), 3.58 (3 H, s, CH₃), 3.35 (3 |
| | H, s, CH₃), 2.50-2.41 (2 H, m, CH₂), 1.17-1.08 (3 H, m, CH₃). |
| 94 | ¹H NMR δ (ppm)(DMSO-d₆): 8.97 (1 H, dd, ArH), 8.59 (1 H, dd, ArH), 8.49 (1 H, d, |
| | ArH), 8.14 (1 H, ddd, ArH), 8.00 (1 H, s, ArH), 7.74 (1 H, d, ArH), 7.66 (1 H, dd, |
| | ArH), 7.51 (1 H, ddd, ArH), 7.31 (1 H, d, ArH), 6.30 (1 H, d, ArH), 3.63 (3 H, s, |
| | CH₃), 3.32 (3 H, s, CH₃), 2.52 (2 H, q, CH₂), 1.16 (3 H, t, CH₃). |
| 95 | ¹H NMR δ (ppm)(DMSO-d₆): 9.23-9.18 (3 H, m, ArH), 8.49 (1 H, s, ArH), 8.01 (1 H, |
| | s, ArH), 7.84-7.82 (1 H, m, ArH), 7.76-7.72 (1 H, m, ArH), 7.37-7.30 (1 H, m, ArH), |
| | 6.33 (1 H, s, ArH), 3.64 (3 H, s, CH₃), 3.38 (3 H, s, CH₃), 2.57-2.51 (2 H, m, CH₂), |
| | 1.20-1.11 (3 H, m, CH₃). |
| 97 | ¹H NMR δ (ppm)(CHCl₃-d): 8.84 (1 H, d, ArH), 8.73 (1 H, d, ArH), 7.98 (1 H, dd, |
| | ArH), 7.78-7.75 (1 H, m, ArH), 7.64 (1 H, s, ArH), 7.31-7.23 (1 H, dd, ArH), 6.95 (1 |
| | H, d, ArH), 6.87 (1 H, d, ArH), 5.97 (1 H, d, ArH), 5.23 (2 H, s, CH₂), 3.59 (3 H, s, |
| | CH₃), 3.44 (3 H, s, CH₃), 2.10 (3 H, s, CH₃). |
| 98 | ¹H NMR δ (ppm)(CHCl₃-d): 8.71 (1 H, d, ArH), 7.86 (1 H, d, ArH), 7.69 (1 H, dd, |
| | ArH), 7.61 (1 H, s, ArH), 7.39 (2 H, m, ArH), 7.32 (2 H, m, ArH), 7.25 (2 H, d, ArH), |
| | 6.58 (1 H, t, NH), 5.91 (1 H, d, ArH), 4.68 (2 H, d, CH₂), 3.56 (3 H, s, CH₃), 3.45 (3 |
| | H, s, CH₃), 2.56 (2 H, q, CH₂), 1.17 (3 H, t, CH₃). |
| 99 | ¹H NMR δ (ppm)(CHCl₃-d): 8.72 (1 H, s, ArH), 7.75 (1 H, s, ArH), 7.62 (1 H, s, |
| | ArH), 7.56 (1 H, dd, ArH), 7.37-7.32 (2 H, m, ArH), 7.29-7.20 (4 H, m, ArH), 6.15 (1 |
| | H, m, NH), 5.90 (1 H, s, ArH), 3.76 (2 H, m, CH₂), 3.57 (3 H, s, CH₃), 3.44 (3 H, s, |
| | CH₃), 2.97 (2 H, t, CH₂), 2.54 (2 H, q, CH₂), 1.16 (3 H, t, CH₃). |
| 100 | ¹H NMR δ (ppm)(CHCl₃-d): 8.73 (1 H, d, ArH), 7.63 (1 H, s, ArH), 7.46 (1 H, d, |
| | ArH), 7.33 (1 H, dd, ArH), 7.25 (1 H, d, ArH), 5.94 (1 H, s, ArH), 3.81 (2 H, s, CH₂), |
| | 3.60 (3 H, s, CH₃), 3.55 (2 H, s, CH₂), 3.45 (3 H, s, CH₃), 2.52 (2 H, q, CH₂), 2.51 (2 |
| | H, s, CH₂), 2.42 (2 H, s, CH₂), 2.35 (3 H, s, CH₃), 1.15 (3 H, t, CH₃). |
| 101 | ¹H NMR δ (ppm)(CHCl₃-d): 8.72 (1 H, s, ArH), 7.60 (1 H, s, ArH), 7.40 (1 H, d, |
| | ArH), 7.31 (1 H, dd, ArH), 7.19 (1 H, d, ArH), 5.89 (1 H, s, ArH), 4.97 (1 H, q, CH), |
| | 3.56 (3 H, s, CH₃), 3.44 (3 H, s, CH₃), 2.54 (2 H, q, CH₂), 1.95 (1 H, s, OH), 1.55 (3 |
| | H, d, CH₃), 1.17 (3 H, t, CH₃). |
| 102 | ¹H NMR δ (ppm)(CHCl₃-d): 8.72 (1 H, d, ArH), 7.82 (1 H, d, ArH), 7.65 (1 H, d, |
| | ArH), 7.52 (1 H, s, ArH), 7.26 (1 H, d, ArH), 6.20 (1 H, m, NH), 5.91 (1 H, d, ArH), |
| | 3.55 (3 H, s, CH₃), 3.51 (2 H, q, CH₂), 3.46 (3 H, s, CH₃), 2.56 (2 H, q, CH₂), 1.29 (3 |
| | H, t, CH₃), 1.18 (3 H, t, CH₃). |
| 103 | ¹H NMR δ (ppm)(CHCl₃-d): 8.71 (1 H, d, ArH), 7.60 (1 H, s, ArH), 7.53 (1 H, d, |
| | ArH), 7.40 (1 H, dd, ArH), 7.16 (1 H, d, ArH), 5.89 (1 H, d, ArH), 3.56 (3 H, s, CH₃), |
| | 3.44 (3 H, s, CH₃), 2.54 (2 H, q, CH₂), 2.01 (1 H, s, OH), 1.66 (6 H, s, CH₃), 1.17 (3 |
| | H, t, CH₃). |
| 104 | ¹H NMR δ (ppm)(CHCl₃-d): 8.71 (1 H, s, ArH), 7.66 (1 H, s, ArH), 7.48 (1 H, s, |
| | ArH), 7.38-7.27 (2 H, m, ArH), 6.00 (1 H, s, ArH), 4.17 (4 H, s, CH₂), 3.61 (3 H, s, |
| | CH₃), 3.45 (3 H, s, CH₃), 3.12 (4 H, s, CH₂), 2.57 (2 H, q, CH₂), 1.18 (3 H, t, CH₃). |
| 105 | ¹H NMR δ (ppm)(CHCl₃-d): 8.73 (1 H, d, ArH), 7.60 (1 H, s, ArH), 7.38 (1 H, d, |
| | ArH), 7.30-7.25 (1 H, m, ArH), 7.19 (1 H, d, ArH), 5.88 (1 H, s, ArH), 4.49 (2 H, s, |
| | CH₂), 3.54 (3 H, s, CH₃), 3.50 (3 H, s, CH₃), 3.44 (3 H, s, CH₃), 2.54 (2 H, q, CH₂), |
| | 1.16 (3 H, t, CH₃). |
| 106 | ¹H NMR δ (ppm)(CHCl₃-d): 8.73 (1 H, s, ArH), 7.63 (1 H, s, ArH), 7.57 (1 H, d, |
| | ArH), 7.44 (1 H, m, ArH), 7.28-7.21 (1 H, m, ArH), 5.92 (1 H, d, ArH), 4.05-3.82 (1 |
| | H, m, CH), 3.70 (1 H, m, CH), 3.59 (3 H, s, CH₃), 3.51-3.34 (4 H, m, CH, CH₃), |
| | 2.85-2.65 (1 H, m, CH), 2.55 (2 H, q, CH₂), 2.33 (3 H, s, CH₃), 2.31-2.03 (1 H, m, CH), |
| | 2.21 (3 H, s, CH₃), 1.84 (2 H, m, CH₂), 1.17 (3 H, t, CH₃). |
| 107 | ¹H NMR δ (ppm)(DMSO-d₆): 9.41 (1 H, s, OH), 8.47 (1 H, s, ArH), 7.92 (1 H, s, |
| | ArH), 6.95 (1 H, d, ArH), 6.75 (1 H, d, ArH), 6.67 (1 H, dd, ArH), 5.94 (1 H, s, ArH), |
| | 3.53 (3 H, s, CH₃), 3.31 (3 H, s, CH₃), 2.34 (2 H, q, CH₂), 1.04 (3 H, t, CH₃). |
| 108 | ¹H NMR δ (ppm)(DMSO-d₆): 8.55 (1 H, s, ArH), 8.38 (1 H, s, ArH), 7.95 (1 H, s, |
| | ArH), 7.88 (1 H, dd, ArH), 7.75 (1 H, d, ArH), 7.52 (1 H, d, ArH), 7.36 (1 H, s, NH), |
| | 6.67 (1 H, s, NH), 3.72 (3 H, s, CH₃), 3.39 (3 H, s, CH₃), 2.53-2.44 (2 H, m, CH₂), |
| | 1.11 (3 H, t, CH₃). |
| 109 | ¹H NMR δ (ppm)(CHCl₃-d): 8.71 (1 H, s, ArH), 7.60 (1 H, s, ArH), 7.39 (1 H, d, |
| | ArH), 7.33 (1 H, d, ArH), 7.21 (1 H, s, ArH), 5.89 (1 H, s, ArH), 4.70 (2 H, s, CH₂), |
| | 3.55 (3 H, s, CH₃), 3.45 (3 H, s, CH₃), 2.52 (2 H, q, CH₂), 1.99 (1 H, m, OH), 1.15 (3 |
| | H, t, CH₃). |
| 110 | ¹H NMR δ (ppm)(DMSO-d₆): 8.48 (1 H, s, ArH), 7.96 (1 H, s, ArH), 7.11 (1 H, d, |
| | ArH), 6.95 (1 H, d, ArH), 6.91-6.86 (1 H, m, ArH), 6.06 (1 H, s, ArH), 5.09 (1 H, s, |
| | CH), 3.66-3.59 (1 H, m, CH), 3.58 (3 H, s, CH₃), 3.32 (3 H, s, CH₃), 2.99-2.87 (3 H, |
| | m, CH), 2.46-2.36 (2 H, m, CH), 2.31-2.20 (1 H, m, CH), 2.19-2.11 (1 H, m, CH), |
| | 1.15-1.05 (3 H, m, CH₃), CH₃ under water peak). |
| 111 | ¹H NMR δ (ppm)(CHCl₃-d): 8.70 (1 H, s, ArH), 7.61 (2 H, d, ArH), 7.20-7.03 (4 H, |
| | m, ArH), 7.00 (1 H, s, ArH), 5.91 (1 H, s, ArH), 5.16 (2 H, s, CH₂), 3.58 (3 H, s, |
| | CH₃), 3.42 (3 H, s, CH₃), 2.51 (2 H, q, CH₂), 1.13 (3 H, t, CH₃). |
| 112 | ¹H NMR δ (ppm)(CHCl₃-d): 8.65 (1 H, s, ArH), 7.70 (1 H, s, ArH), 7.44 (1 H, s, |
| | ArH), 7.33 (1 H, d, ArH), 7.27-7.24 |
| 113 | ¹H NMR δ (ppm)(CHCl₃-d): 8.69 (1 H, s, ArH), 7.73-7.64 (2 H, m, ArH), 7.54 (1 H, |
| | dd, ArH), 7.25 (1 H, m, ArH), 5.96 (1 H, s, ArH), 4.03 (2 H, s, CH), 3.92 (2 H, s, CH), |
| | 3.59 (3 H, s, CH₃), 3.46 (3 H, s, CH₃), 2.54 (2 H, q, CH₂), 1.33 (6 H, s, CH₃), 1.18 (3 |
| | H, t, CH₃). |
| 114 | ¹H NMR δ (ppm)(DMSO-d₆): 9.16 (1 H, d, ArH), 8.48-8.45 (1 H, m, ArH), 8.41 (1 |
| | H, dd, ArH), 8.17-8.10 (1 H, m, ArH), 7.99 (1 H, s, ArH), 7.83 (1 H, d, ArH), 7.75 (1 |
| | H, dd, ArH), 7.34 (1 H, d, ArH), 6.34 (1 H, s, ArH), 3.63 (3 H, s, CH₃), 3.36 (3 H, s, |
| | CH₃), 2.54-2.46 (2 H, m, CH₂), 1.20-1.10 (3 H, m, CH₃). |
| 115 | ¹H NMR δ (ppm)(DMSO-d₆): 10.65 (1 H, s, NH), 9.03 (2 H, s, ArH), 8.48 (1 H, d, |
| | ArH), 8.01-7.94 (1 H, m, ArH), 7.76 (1 H, d, ArH), 7.67 (1 H, dd, ArH), 7.29 (1 H, d, |
| | ArH), 6.28 (1 H, s, ArH), 3.62 (3 H, s, CH₃), 3.36 (3 H, s, CH₃), 2.53-2.44 (2 H, m, |
| | CH₂), 2.21 (3 H, s, CH₃), 1.17-1.08 (3 H, m, CH₃). |
| 116 | ¹H NMR δ (ppm)(CHCl₃-d): 8.65 (1 H, s, ArH), 7.75-7.64 (3 H, m, ArH), 7.45 (1 H, |
| | d, ArH), 6.59 (1 H, m, NH), 6.00 (1 H, s, ArH), 3.67-3.59 (5 H, m, CH₂, CH₃), 3.56 (2 |
| | H, t, CH₂), 3.59-3.37 (3 H, m, CH₃), 3.37 (3 H, s, CH₃), 2.55 (2 H, q, CH₂), 1.17 (3 H, |
| | t, CH₃). |
| 117 | ¹H NMR δ (ppm)(CHCl₃-d): 8.67 (1 H, s, ArH), 7.70 (1 H, s, ArH), 7.46 (1 H, d, |
| | ArH), 7.40 (1 H, d, ArH), 7.26 (1 H, d, ArH), 6.05 (1 H, s, ArH), 3.90-3.45 (8 H, m, |
| | CH₂), 3.62 (3 H, s, CH₃), 3.46 (3 H, s, CH₃), 2.56 (2 H, q, CH₂), 1.17 (3 H, t, CH₃). |
| 118 | ¹H NMR δ (ppm)(CHCl₃-d): 8.58 (1 H, s, ArH), 7.76 (1 H, s, ArH), 7.46 (1 H, m, |
| | ArH), 7.36 (1 H, dd, ArH), 7.25 (1 H, m, ArH), 6.15 (1 H, s, ArH), 3.65 (3 H, s, CH₃), |
| | 3.47 (3 H, s, CH₃), 3.14 (3 H, s, CH₃), 3.06 (3 H, s, CH₃), 2.54 (2 H, q, CH₂), 1.16 (3 |
| | H, t, CH₃). |
| 119 | ¹H NMR δ (ppm)(CHCl₃-d): 8.72 (1 H, s, ArH), 7.87 (1 H, s, ArH), 7.70 (1 H, d, |
| | ArH), 7.63 (1 H, s, ArH), 7.26 (1 H, d, ArH), 7.00 (1 H, s, NH), 5.92 (1 H, s, ArH), |
| | 3.60-3.53 (2 H, m, CH₂), 3.55 (3 H, s, CH₃), 3.45 (3 H, s, CH₃), 2.60-2.51 (4 H, m |
| | CH₂), 2.30 (6 H, s, CH₃), 1.18 (3 H, t, CH₃). |
| 120 | ¹H NMR δ (ppm)(DMSO-d₆): 9.22 (1 H, m, NH), 8.48 (1 H, s, ArH), 8.01 (1 H, s, |
| | ArH), 7.89 (1 H, s, ArH), 7.80 (1 H, dd, ArH), 7.31 (1 H, d, ArH), 6.29 (1 H, s, ArH), |
| | 4.34 (2 H, d, CH₂), 3.63 (3 H, s, CH₃), 3.36 (3 H, s, CH₃), 2.50 (2 H, q, CH₂), 1.12 (3 |
| | H, t, CH₃). |
| 121 | ¹H NMR δ (ppm)(DMSO-d₆): 9.00 (2 H, s, ArH), 8.49 (1 H, d, ArH), 8.00 (1 H, s, |
| | ArH), 7.74 (1 H, d, ArH), 7.65 (1 H, dd, ArH), 7.30 (1 H, d, ArH), 6.30 (1 H, s, ArH), |
| | 3.99 (3 H, s, CH₃), 3.63 (3 H, s, CH₃), 3.35 (2 H, s, CH₂), 2.52 (3 H, s, CH₃), 1.15 (3 |
| | H, t, CH₃). |
| 122 | ¹H NMR δ (ppm)(DMSO-d₆): 8.49 (1 H, d, ArH), 7.98 (1 H, s, ArH), 7.37 (1 H, d, |
| | ArH), 7.24 (1 H, dd, ArH), 7.12 (1 H, d, ArH), 6.13 (1 H, d, ArH), 4.39 (2 H, s, CH₂), |
| | 3.59 (3 H, s, CH₃), 3.39-3.24 (5 H, m, CH₂, CH₃), 2.54-2.41 (3 H, m, CH₃), 1.09 (3 |
| | H, t, CH₃). |
| 123 | ¹H NMR δ (ppm)(CHCl₃-d): 8.72 (1 H, d, ArH), 7.69 (1 H, dd, ArH), 7.62 (1 H, s, |
| | ArH), 7.56 (1 H, d, ArH), 7.45 (1 H, m, ArH), 5.90 (2 H, d, ArH, NH), 3.57 (3 H, s, |
| | CH₃), 3.46 (3 H, s, CH₃), 2.63-2.50 (2 H, m, CH₂), 1.46 (9 H, s, CH₃), 1.17 (3 H, q, |
| | CH₃). |
| 124 | ¹H NMR δ (ppm)(CHCl₃-d): 8.69-8.67 (2 H, m, ArH), 8.38 (1 H, dd, ArH), 8.29 (1 H, |
| | ddd, ArH), 8.21 (1 H, s, ArH), 7.87 (1 H, dd, ArH), 7.80 (1 H, d, ArH), 7.62 (1 H, s, |
| | ArH), 7.53 (1 H, d, ArH), 7.36-7.24 (1 H, m ArH), 5.92 (1 H, d, NH), 3.58 (3 H, s, |
| | CH₃), 3.46 (3 H, s, CH₃), 2.60 (2 H, q, CH₂), 1.19 (3 H, t, CH₃). |
| 125 | ¹H NMR δ (ppm)(CHCl₃-d): 8.72 (1 H, d, ArH), 8.55 (1 H, m, NH), 7.83 (1 H, dd, |
| | ArH), 7.79-7.58 (3 H, m, ArH), 7.62-7.41 (2 H, m, ArH), 7.33 (1 H, d, ArH), |
| | 7.29-7.15 (1 H, m, ArH), 5.88 (1 H, d, ArH), 4.75 (2 H, d, CH₂), 3.56 (3 H, s, CH₃), 3.48 (3 |
| | H, s, CH₃), 2.63-2.53 (2 H, m, CH₂), 1.17 (3 H, t, CH₃). |
| 126 | ¹H NMR δ (ppm)(DMSO-d₆): 8.50 (1 H, d, ArH), 7.99 (1 H, s, ArH), 7.66 (1 H, d, |
| | ArH), 7.60-7.53 (3 H, m, ArH), 7.25-7.13 (2 H, m, ArH), 6.23 (1 H, s, ArH), 3.61 (3 |
| | H, s, CH₃), 3.36 (3 H, s, CH₃), 2.54-2.46 (2 H, m, CH₂), 1.13 (3 H, t, CH₃). |
| 127 | ¹H NMR δ (ppm)(DMSO-d₆): 8.50 (1 H, d, ArH), 7.98 (1 H, s, ArH), 7.91 (1 H, dd, |
| | ArH), 7.73 (1 H, d, ArH), 7.68-7.60 (3 H, m, ArH), 7.22 (1 H, d, ArH), 6.19 (1 H, d, |
| | ArH), 3.59 (3 H, s, CH₃), 3.37 (3 H, s, CH₃), 2.54-2.46 (2 H, m, CH₂), 1.14 (3 H, t, |
| | CH₃). |
| 128 | ¹H NMR δ (ppm)(CHCl₃-d): 8.68 (1 H, d, ArH), 7.75 (1 H, dd, ArH), 7.64-7.59 (1 H, |
| | m, ArH), 7.45 (1 H, d, ArH), 7.27 (1 H, s, ArH), 6.40 (1 H, s, NH), 5.87 (1 H, d, |
| | ArH), 3.55 (3 H, s, CH₃), 3.44 (3 H, s, CH₃), 2.99 (3 H, d, CH₃), 2.62-2.51 (2 H, m, |
| | CH₂), 1.17 (3 H, t, CH₃). |
| 129 | ¹H NMR δ (ppm)(DMSO-d₆): 8.75 (2 H, s, ArH), 8.48 (1 H, d, ArH), 7.97 (1 H, s, |
| | ArH), 7.62 (1 H, d, ArH), 7.54 (1 H, dd, ArH), 7.22 (1 H, d, ArH), 6.23 (1 H, d, ArH), |
| | 3.60 (3 H, s, CH₃), 3.36 (3 H, s, CH₃), 3.18 (6 H, s, CH₃), 2.52-2.44 (2 H, m, CH₂), |
| | 1.19-1.09 (3 H, m, CH₃). |
| 130 | ¹H NMR δ (ppm)(DMSO-d₆): 8.55-8.54 (1 H, m, ArH), 8.49-8.48 (1 H, m, ArH), |
| | 8.10-8.06 (1 H, m, ArH), 7.99 (1 H, s, ArH), 7.67-7.65 (1 H, m, ArH), 7.61-7.57 (1 H, |
| | m, ArH), 7.27 (1 H, d, ArH), 6.95-6.92 (1 H, m, ArH), 6.26 (1 H, s, ArH), 3.92 (3 H, |
| | s, CH₃), 3.62 (3 H, s, CH₃), 3.37 (3 H, s, CH₃), 1.15 (3 H, t, CH₃), (CH₂ under DMSO |
| | peak). |
| 131 | ¹H NMR δ (ppm)(CHCl3-d): 8.79 (1 H, s, ArH), 8.74 (1 H, s, ArH), 7.86-7.82 (1 H, |
| | m, ArH), 7.63 (1 H, s, ArH), 7.58 (1 H, d, ArH), 7.53-7.49 (1 H, m. ArH), 7.31 (1 H, |
| | s, ArH), 7.29 (1 H, s, ArH), 7.24 (1 H, s, ArH), 3.59 (3 H, s, CH₃), 3.48 (3 H, s, CH₃), |
| | 2.63-2.56 (5 H, m, CH₂, CH₃), 1.21 (3 H, t, CH₃). |
| 132 | ¹H NMR δ (ppm)(DMSO-d₆): 8.49-8.48 (1 H, m, ArH), 7.98 (1 H, s, ArH), |
| | 7.92-7.87 (1 H, m, ArH), 7.78-7.76 (1 H, m, ArH), 7.57-7.55 (1 H, m, ArH), 7.51-7.47 (1 H, m, |
| | ArH), 7.22-7.18 (1 H, m, ArH), 6.47-6.42 (1 H, m, ArH), 6.20 (1 H, s, ArH), 3.61 (3 |
| | H, s, CH₃), 2.50-2.43 (2 H, m, CH₂), 1.17-1.08 (3 H, m, CH₃), (CH₃ under water |
| | peak), (OH not observed). |
| 133 | ¹H NMR δ (ppm)(CHCl₃-d): 8.74 (2 H, s, ArH), 8.48 (1 H, s, ArH), 7.68-7.59 (3 H, |
| | m, ArH), 7.54-7.50 (1 H, m, ArH), 7.34 (1 H, d, ArH), 6.00 (1 H, s, ArH), 3.60 (3 H, |
| | s, CH₃), 3.49 (3 H, s, CH₃), 2.61 (2 H, q, CH₂), 1.22 (3 H, t, CH₃). |
| 134 | ¹H NMR δ (ppm)(DMSO-d₆): 12.22 (1 H, s, OH), 8.68 (2 H, s, ArH), 8.47 (1 H, d, |
| | ArH), 7.98 (1 H, s, ArH), 7.63 (1 H, d, ArH), 7.54 (1 H, dd, ArH), 7.22 (1 H, d, ArH), |
| | 6.22 (1 H, s, ArH), 3.60 (3 H, s, CH₃), 3.35 (3 H, s, CH₃), 2.52-2.44 (2 H, m, CH₂), |
| | 1.12 (3 H, t, CH₃). |
| 135 | ¹H NMR δ (ppm)(CHCl₃-d): 8.73 (1 H, d, ArH), 7.60 (1 H, s, ArH), 7.35 (1 H, d, |
| | ArH), 7.28-7.23 (1 H, m, ArH), 7.17 (1 H, d, ArH), 5.87 (1 H, d, ArH), 3.80 (2 H, s, |
| | CH₂), 3.55 (3 H, s, CH₃), 3.45 (3 H, s, CH₃), 2.57-2.48 (5 H, m, CH₂, CH₃), 1.16 (3 H, |
| | t, CH₃), (NH not observed). |
| 136 | ¹H NMR δ (ppm)(DMSO-d₆): 8.47 (1 H, d, ArH), 7.96 (1 H, s, ArH), 7.30 (1 H, d, |
| | ArH), 7.23 (1 H, dd, ArH), 7.12 (1 H, d, ArH), 6.10 (1 H, s, ArH), 3.59 (3 H, s, CH₃), |
| | 3.57 (2 H, s, CH₂), 3.33 (3 H, s, CH₃), 3.15-3.11 (4 H, m, CH), 2.87 (3 H, s, CH₃), |
| | 2.52-2.39 (6 H, m, CH₂, CH), 1.07 (3 H, t, CH₃). |
| 137 | ¹H NMR δ (ppm)(DMSO-d₆): 8.47 (1 H, d, ArH), 8.01-7.94 (2 H, m, ArH, NH), |
| | 7.88 (1 H, d, ArH), 7.78 (1 H, dd, ArH), 7.33 (1 H, s, NH), 7.23 (1 H, d, ArH), 6.24 (1 H, |
| | d, ArH), 3.60 (3 H, s, CH₃), 3.34 (3 H, s, CH₃), 2.52-2.42 (2 H, m, CH₂), 1.10 (3 H, t, |
| | CH₃). |
| 138 | ¹H NMR δ (ppm)(DMSO-d₆): 8.49 (1 H, d, ArH), 7.99 (1 H, s, ArH), 7.24-7.15 (3 H, |
| | m, ArH), 6.24 (1 H, s, ArH), 3.72 (3 H, s, CH₃), 3.62 (3 H, s, CH₃), 3.35 (3 H, d, |
| | CH₃), 2.53-2.44 (2 H, m, CH₂), 2.28 (3 H, s, CH₃), 2.19 (3 H, s, CH₃), 1.12 (3 H, t, |
| | CH₃). |
| 139 | ¹H NMR δ (ppm)(DMSO-d₆): 8.50 (1 H, d, ArH), 8.34 (1 H, s, ArH), 7.98-7.96 (2 H, |
| | m, ArH), 7.60 (1 H, d, ArH), 7.51 (1 H, dd, ArH), 7.41-7.27 (5 H, m, ArH), 7.16 (1 |
| | H, m, ArH), 6.11 (1 H, s, ArH), 5.37 (2 H, s, CH₂), 3.57 (3 H, s, CH₃), 3.35 (3 H, t, |
| | CH₃), 2.56-2.41 (2 H, m, CH₂), 1.13 (3 H, t, CH₃). |
| 140 | ¹H NMR δ (ppm)(DMSO-d₆): 9.11 (2 H, s, ArH), 8.49 (1 H, s, ArH), 8.01 (1 H, s, |
| | ArH), 7.78-7.76 (1 H, m, ArH), 7.71-7.67 (1 H, m, ArH), 7.36-7.30 (1 H, m, ArH), |
| | 6.33 (1 H, s, ArH), 3.64 (3 H, s, CH₃), 3.38 (3 H, s, CH₃), 3.25-3.17 (1 H, m, CH), |
| | 2.55-2.48 (2 H, m, CH₂), 1.34 (6 H, d, CH₃), 1.15 (3 H, t, CH₃). |
| 141 | ¹H NMR δ (ppm)(DMSO-d₆): 8.48 (1 H, s, ArH), 7.97 (1 H, s, ArH), 7.30-7.28 (1 H, |
| | m, ArH), 7.24-7.20 (1 H, m, ArH), 7.15-7.08 (1 H, m, ArH), 6.11-6.10 (1 H, m, ArH), |
| | 3.58 (3 H, s, CH₃), 3.48 (2 H, s, CH₂), 2.53-2.28 (10 H, m, CH), 2.17 (3 H, s, CH₃), |
| | 1.12-1.03 (3 H, m, CH₃), (CH₃ under water peak). |
| 142 | ¹H NMR δ (ppm)(DMSO-d₆): 8.48 (1 H, s, ArH), 7.97 (1 H, s, ArH), 7.30 (1 H, s, |
| | ArH), 7.26-7.22 (1 H, m, ArH), 7.15-7.11 (1 H, m, ArH), 6.11 (1 H, s, ArH), |
| | 3.64-3.56 (7 H, m, CH), 3.50 (2 H, s, CH₂), 2.48-2.37 (6 H, m, CH), 1.09 (3 H, t, CH₃), |
| | (CH₃ under water peak). |
| 143 | ¹H NMR δ (ppm)(DMSO-d₆): 8.48 (1 H, s, ArH), 7.98 (1 H, s, ArH), 7.34 (1 H, s, |
| | ArH), 7.29-7.25 (1 H, m, ArH), 7.17-7.13 (1 H, m, ArH), 6.12 (1 H, s, ArH), 3.71 (2 |
| | H, s, CH₂), 3.59 (3 H, s, CH₃), 3.17-3.12 (4 H, m, CH), 2.93 (4 H, s, CH), |
| | 2.47-2.40 (2 H, m, CH₂), 1.09 (3 H, t, CH₃), (CH₃ under water peak). |
| 144 | ¹H NMR δ (ppm)(CHCl₃-d): 9.07 (1 H, d, ArH), 8.74 (1 H, d, ArH), 8.66 (1 H, dd, |
| | ArH), 8.53 (1 H, m, ArH), 8.06 (1 H, d, ArH), 7.92 (1 H, dd, ArH), 7.62 (1 H, s, |
| | ArH), 7.36 (1 H, d, ArH), 5.97 (1 H, d, ArH), 3.56 (3 H, s, CH₃), 3.50 (3 H, s, CH₃), |
| | 2.62 (2 H, q, CH₂), 1.23 (3 H, t, CH₃). |
| 145 | ¹H NMR δ (ppm)(DMSO-d₆): 8.48 (1 H, d, ArH), 7.95 (1 H, s, ArH), 7.75 (1 H, d, |
| | ArH), 7.65-7.60 (3 H, m, ArH), 7.13 (1 H, d, ArH), 6.09 (1 H, s, ArH), 3.69 (3 H, s, |
| | CH₃), 3.55 (3 H, s, CH₃), 3.40-3.32 (3 H, m, CH₃), 2.48-2.39 (2 H, m, CH₂), |
| | 1.18-1.07 (3 H, m, CH₃). |
| 146 | ¹H NMR δ (ppm)(DMSO-d₆): 8.47 (1 H, d, ArH), 7.95 (1 H, s, ArH), 7.35 (1 H, d, |
| | ArH), 7.27 (1 H, dd, ArH), 7.10 (1 H, d, ArH), 6.08 (1 H, s, ArH), 3.71 (3 H, s, CH₃), |
| | 3.56 (3 H, s, CH₃), 3.20-3.11 (2 H, m, CH), 3.05-2.96 (2 H, m, CH), 2.81 (1 H, s, |
| | CH), 2.52-2.38 (4 H, m, CH), 2.13-2.04 (2 H, m, CH), 1.95 (2 H, s, CH), 1.08 (3 H, t, |
| | CH₃), (NH not observed). |
| 147 | ¹H NMR δ (ppm)(DMSO-d₆): 8.47 (1 H, d, ArH), 8.01-7.95 (2 H, m, ArH), 7.90 (1 |
| | H, dd, ArH), 7.35 (1 H, d, ArH), 6.34 (1 H, s, ArH), 3.63 (3 H, s, CH₃), 3.37 (3 H, s, |
| | CH₃), 2.68 (3 H, s, CH₃), 2.53-2.48 (2 H, m, CH₂), 1.12 (3 H, t, CH₃). |
| 148 | ¹H NMR δ (ppm)(DMSO-d₆): 8.48 (1 H, d, ArH), 8.02-7.96 (2 H, m, ArH), 7.92 (1 |
| | H, dd, ArH), 7.38 (1 H, d, ArH), 6.37 (1 H, d, ArH), 4.82 (2 H, s, CH), 3.63 (3 H, s, |
| | CH₃), 3.37 (3 H, s, CH₃), 2.56-2.47 (2 H, m, CH₂), 1.12 (3 H, t, CH₃). |
| 149 | ¹H NMR δ (ppm)(DMSO-d₆): 8.49 (1 H, s ArH), 7.97 (1 H, s, ArH), 7.36-7.34 (1 H, |
| | m, ArH), 7.28-7.24 (1 H, m, ArH), 7.15-7.12 (1 H, m, ArH), 6.11 (1 H, s, ArH), |
| | 3.81-3.76 (2 H, m, CH), 3.68-3.62 (2 H, m, CH), 3.57 (3 H, s, CH₃), 3.09-3.04 (1 H, m, |
| | NH), 2.49-2.39 (2 H, m, CH), 1.13-1.05 (3 H, m, CH₃), (CH₃ under water peak). |
| 150 | ¹H NMR δ (ppm)(DMSO-d₆): 9.21 (1 H, d, ArH), 8.50-8.48 (1 H, m, ArH), 8.19 (1 |
| | H, d, ArH), 8.04-7.99 (1 H, m, ArH), 7.97 (1 H, s, ArH), 7.90 (1 H, dd, ArH), 7.25 (1 |
| | H, d, ArH), 6.18 (1 H, d, ArH), 3.58 (3 H, s, CH₃), 3.36 (3 H, s, CH₃), 1.19-1.08 (3 H, |
| | m, CH₃), (CH₂ under DMSO peak). |
| 151 | ¹H NMR δ (ppm)(DMSO-d₆): 9.09 (1 H, d, ArH), 8.47 (1 H, d, ArH), 8.35-8.33 (1 H, |
| | m, ArH), 7.98 (1 H, s, ArH), 7.67 (1 H, d, ArH), 7.60 (1 H, dd, ArH), 7.24 (1 H, d, |
| | ArH), 6.26 (1 H, d, ArH), 3.61 (3 H, s, CH₃), 2.48-2.42 (2 H, m, CH₂), 1.12 (3 H, t, |
| | CH₃), (CH₃ under water peak). |
| 152 | ¹H NMR δ (ppm)(DMSO-d₆): 8.47 (1 H, d, NH), 8.15 (1 H, d, ArH), 7.98 (1 H, s, |
| | ArH), 7.84 (1 H, d, ArH), 7.77-7.71 (1 H, m, ArH), 7.24 (1 H, d, ArH), 6.21 (1 H, s, |
| | ArH), 3.99-3.89 (1 H, m, CH), 3.59 (3 H, s, CH₃), 3.34 (3 H, s, CH₃), 2.80 (1 H, d, |
| | CH), 2.64 (1 H, d, CH), 2.49-2.41 (2 H, m, CH), 2.17 (3 H, s, CH₃), 1.91-1.73 (3 H, |
| | m, CH), 1.72-1.64 (1 H, m, CH), 1.58-1.45 (1 H, m, CH), 1.37-1.24 (1 H, m, CH), |
| | 1.10 (3 H, t, CH₃). |
| 153 | ¹H NMR δ (ppm)(CHCl₃-d): 8.73 (1 H, d, ArH), 7.63 (1 H, s, ArH), 7.42 (1 H, d, |
| | ArH), 7.35 (1 H, dd, ArH), 7.23-7.15 (1 H, m, ArH), 6.22-6.20 (1 H, m, CH), 5.91 (1 |
| | H, d, ArH), 4.38-4.35 (2 H, m, CH), 4.00-3.94 (2 H, m, CH), 3.56 (3 H, s, CH₃), |
| | 3.45 (3 H, s, CH₃), 2.66-2.50 (4 H, m, CH), 1.25-1.13 (3 H, m, CH₃). |
| 154 | ¹H NMR δ (ppm)(CHCl₃-d): 8.72 (1 H, s, ArH), 7.61 (1 H, s, ArH), 7.43-7.38 (1 H, |
| | m, ArH), 7.35-7.30 (1 H, m, ArH), 7.19-7.15 (1 H, m, ArH), 6.13 (1 H, s, CH), |
| | 5.90 (1 H, d, ArH), 4.11 (2 H, s, CH), 3.70-3.64 (2 H, m, CH), 3.56 (3 H, s, CH₃), 3.45 (3 |
| | H, s, CH₃), 2.62-2.47 (4 H, m, CH), 1.54-1.50 (9 H, m, C(CH₃)3), 1.29-1.11 (3 H, m, |
| | CH₃). |
| 155 | ¹H NMR δ (ppm)(DMSO-d₆): 8.68 (1 H, s, ArH), 8.56 (1 H, d, ArH), 8.50 (1 H, d, |
| | ArH), 8.02 (1 H, s, ArH), 7.70 (1 H, d, ArH), 7.52 (1 H, d, ArH), 7.46 (1 H, m, ArH), |
| | 7.31 (1 H, d, ArH), 6.36 (1 H, s, ArH), 3.66 (3 H, s, CH₃), 3.39 (3 H, s, CH₃), 1.13 (3 |
| | H, t, CH₃), (CH₂ under DMSO peak). |
| 156 | ¹H NMR δ (ppm)(DMSO-d₆): 8.48 (1 H, d, ArH), 8.00-7.97 (2 H, m, ArH), 7.92 (1 |
| | H, dd, ArH), 7.38 (1 H, d, ArH), 6.33 (1 H, d, ArH), 3.62 (3 H, s, CH₃), 3.45-3.40 (2 |
| | H, m, CH), 3.31 (3 H, s, CH₃), 3.13-3.05 (2 H, m, CH), 2.55-2.48 (2 H, m, CH₂), |
| | 1.12 (3 H, t, CH₃). |
| 157 | ¹H NMR δ (ppm)(DMSO-d₆): 8.48 (1 H, d, ArH), 8.00 (2 H, s, ArH), 7.93 (1 H, dd, |
| | ArH), 7.37 (1 H, d, ArH), 6.35 (1 H, d, ArH), 4.84 (2 H, s, CH), 3.63 (3 H, s, CH₃), |
| | 3.44 (3 H, s, CH₃), 3.39 (3 H, s, CH₃), 2.54-2.46 (2 H, m, CH₂), 1.12 (3 H, t, CH₃). |
| 158 | ¹H NMR δ (ppm)(CHCl₃-d): 8.73 (1 H, d, ArH), 7.61 (1 H, s, ArH), 7.39 (2 H, s, |
| | ArH), 7.18 (1 H, d, ArH), 6.18 and 6.10 (1 H, 2 s, CH), 5.91 (1 H, s, ArH), 4.29 (1 H, |
| | d, CH), 4.17 (1 H, d, CH), 3.89-3.85 (1 H, m, CH), 3.72-3.67 (1 H, m, CH), |
| | 3.59-3.55 (3 H, m, CH₃), 3.45 (3 H, s, CH₃), 2.64 (2 H, d, CH), 2.60 (2 H, s, CH), 2.54 (2 H, m, |
| | CH₂), 1.24-1.13 (6 H, m, CH₃), (rotamers observed). |
| 159 | ¹H NMR δ (ppm)(DMSO-d₆): 8.47 (1 H, s, ArH), 7.99 (1 H, s, ArH), 7.50 (1 H, s, |
| | ArH), 7.41 (1 H, d, ArH), 7.21 (1 H, d, ArH), 6.84 (1 H, s, OH), 6.23 (1 H, s, ArH), |
| | 5.19 (1 H, m, CH), 3.62 (3 H, s, CH₃), 3.33 (3 H, s, CH₃), 2.46 (2 H, q, CH₂), 1.09 (3 |
| | H, t, CH₃). |
| 160 | ¹H NMR δ (ppm)(CHCl₃-d): 8.71 (1 H, s, ArH), 7.84 (1 H, s, ArH), 7.67 (1 H, d, |
| | ArH), 7.63 (1 H, s, ArH), 7.26 (1 H, d, ArH), 6.75 (1 H, m, NH), 5.92 (1 H, s, ArH), |
| | 3.87 (2 H, t, CH₂), 3.70-3.63 (2 H, m, CH₂), 3.57 (3 H, s, CH₃), 3.45 (3 H, s, CH₃), |
| | 2.55 (2 H, q, CH₂), 1.17 (3 H, t, CH₃), (OH not observed). |
| 161 | ¹H NMR δ (ppm)(CHCl₃-d): 8.73 (1 H, s, ArH), 7.85 (1 H, s, ArH), 7.69 (1 H, d, |
| | ArH), 7.63 (1 H, s, ArH), 7.27 (1 H, d, ArH), 6.66 (1 H, m, NH), 5.92 (1 H, s, ArH), |
| | 3.58 (3 H, s, CH₃), 3.52 (2 H, d, CH₂), 3.46 (3 H, s, CH₃), 2.56 (2 H, q, CH₂), 2.42 (1 |
| | H, s, OH), 1.33 (6 H, s, CH₃), 1.18 (3 H, t, CH₃). |
| 162 | ¹H NMR δ (ppm)(DMSO-d₆): 8.49 (1 H, d, ArH), 8.03-7.90 (5 H, m, ArH), 7.76 (1 |
| | H, d, ArH), 7.69 (1 H, dd, ArH), 7.32 (1 H, d, ArH), 6.32 (1 H, d, ArH), 3.64 (3 H, s, |
| | CH₃), 3.35 (3 H, d, CH₃), 2.56-2.48 (2 H, m, CH₂), 1.15 (3 H, t, CH₃). |
| 163 | ¹H NMR δ (ppm)(DMSO-d₆): 8.49 (1 H, d, ArH), 8.25 (1 H, m, ArH), 8.11-8.08 (1 |
| | H, m, ArH), 8.00 (1 H, s, ArH), 7.84 (1 H, m, ArH), 7.78 (1 H, d, ArH), 7.72-7.65 (2 |
| | H, m, ArH), 7.30 (1 H, d, ArH), 6.30 (1 H, d, ArH), 3.63 (3 H, s, CH₃), 3.38 (3 H, s, |
| | CH₃), 2.56-2.48 (2 H, m, CH₂), 1.16 (3 H, t, CH₃). |
| 164 | ¹H NMR δ (ppm)(DMSO-d₆): 8.50 (1 H, d, ArH), 7.99 (1 H, s, ArH), 7.75-7.70 (2 H, |
| | m, ArH), 7.67 (1 H, d, ArH), 7.59 (1 H, dd, ArH), 7.49 (2 H, t, ArH), 7.43-7.35 (1 H, |
| | m, ArH), 7.27 (1 H, d, ArH), 6.26 (1 H, s, ArH), 3.62 (3 H, s, CH₃), 3.38 (3 H, s, |
| | CH₃), 2.55-2.47 (2 H, m, CH₂), 1.15 (3 H, t, CH₃). |
| 165 | ¹H NMR δ (ppm)(DMSO-d₆): 8.50-8.45 (1 H, m, NH), 8.42-8.35 (1 H, m, ArH), |
| | 7.99 (1 H, s, ArH), 7.85 (1 H, d, ArH), 7.76 (1 H, dd, ArH), 7.25 (1 H, d, ArH), 6.25 (1 H, |
| | s, ArH), 3.73-3.43 (3 H, m, CH₃), 3.34 (3 H, s, CH₃), 3.31-3.21 (1 H, m, CH), 2.85 (1 |
| | H, d, CH), 2.49-2.41 (2 H, m, CH), 2.38 (3 H, s, CH₃), 2.29 (2 H, s, CH), 2.24-2.15 (1 |
| | H, m, CH), 1.72-1.65 (2 H, m. CH), 1.57-1.41 (2 H, m, CH), 1.37-1.19 (2 H, m, CH), |
| | 1.14-1.05 (3 H, m, CH₃). |
| 166 | ¹H NMR δ (ppm)(DMSO-d₆): 8.71 (1 H, s, NH), 8.47 (1 H, d, ArH), 7.99 (1 H, s, |
| | ArH), 7.85 (1 H, d, ArH), 7.78 (1 H, dd, ArH), 7.29 (1 H, d, ArH), 6.26 (1 H, s, ArH), |
| | 3.61 (3 H, s, CH₃), 3.35 (3 H, s, CH₃), 2.71 (2 H, s, CH), 2.45 (2 H, d, CH), 2.37 (2 H, |
| | d, CH), 2.26 (2 H, d, CH), 2.20 (3 H, s, CH₃), 1.98 (2 H, s, CH), 1.15-1.06 (3 H, m, |
| | CH₃). |
| 167 | ¹H NMR δ (ppm)(DMSO-d₆): 8.47 (1 H, d, ArH), 7.98-7.93 (1 H, m, ArH), 7.32 (1 |
| | H, d, ArH), 7.23 (1 H, dd, ArH), 7.09 (1 H, d, ArH), 6.02 (1 H, s, ArH), 4.60-4.53 (2 |
| | H, m, CH), 4.33-4.26 (2 H, m, CH), 3.95-3.86 (1 H, m, CH), 3.63 (3 H, s, CH₃), |
| | 3.54 (3 H, s, CH₃), 2.94 (1 H, s, NH), 2.51-2.38 (4 H, m, CH), 1.14-1.04 (3 H, m, CH₃). |
| 168 | ¹H NMR δ (ppm)(CHCl₃-d): 8.73 (1 H, d, ArH), 7.63 (1 H, s, ArH), 7.39 (1 H, d, |
| | ArH), 7.26 (1 H, s, ArH), 7.18 (1 H, dd, ArH), 6.16 and 6.09 (1 H, 2 s, CH), 5.91 (1 |
| | H, d, ArH), 4.28 (1 H, s, CH), 4.17 (1 H, s, CH), 3.86 (1 H, m, CH), 3.70 (1 H, m, |
| | CH), 3.56 (3 H, s, CH₃), 3.45 (3 H, s, CH₃), 2.66 (2 H, m, CH), 2.54 (2 H, q, CH₂), |
| | 2.19 (3 H, s, CH₃), 1.21-1.15 (3 H, m, CH₃), (rotamers observed). |
| 169 | ¹H NMR δ (ppm)(CHCl₃-d): 8.72 (1 H, d, ArH), 7.61 (1 H, s, ArH), 7.34 (1 H, s, |
| | ArH), 7.31 (1 H, m, ArH), 7.23 (1 H, m, ArH), 7.17 (1 H, d, ArH), 5.88 (1 H, s, NH), |
| | 3.89 (2 H, s, CH₂), 3.56 (3 H, s, CH₃), 3.45 (3 H, s, CH₃), 3.10 (1 H, s, OH), 2.65 (2 |
| | H, s, CH₂), 2.53 (2 H, q, CH₂), 1.22 (6 H, s, CH₃), 1.16 (3 H, t, CH₃). |
| 170 | ¹H NMR δ (ppm)(CHCl₃-d): 8.69 (1 H, s, ArH), 7.84 (1 H, s, ArH), 7.71 (1 H, dd, |
| | ArH), 7.64 (1 H, s, ArH), 7.25 (1 H, d, ArH), 7.02 (1 H, d, NH), 5.94 (1 H, s, ArH), |
| | 4.89-4.78 (1 H, m, CH), 4.35 (2 H, m, CH), 3.89 (2 H, dd, CH), 3.58 (3 H, s, CH₃), |
| | 3.44 (3 H, s, CH₃), 2.54 (2 H, q, CH₂), 1.45 (9 H, s, CH₃), 1.16 (3 H, t, CH₃). |
| 171 | ¹H NMR δ (ppm)(DMSO-d₆): 9.09 (1 H, d, NH), 8.47 (1 H, d, ArH), 7.99 (1 H, s, |
| | ArH), 7.88 (1 H, d, ArH), 7.81-7.74 (1 H, m, ArH), 7.27 (1 H, d, ArH), 6.25 (1 H, s, |
| | ArH), 5.08-4.95 (1 H, m, CH), 4.78 (2 H, t, CH), 4.61 (2 H, t, CH), 3.81-3.39 (3 H, |
| | m, CH₃), 3.34 (3 H, s, CH₃), 2.48-2.41 (2 H, m, CH₂), 1.11 (3 H, t, CH₃). |
| 172 | ¹H NMR δ (ppm)(CHCl₃-d): 8.72 (1 H, d, ArH), 7.61 (1 H, s, ArH), 7.40 (1 H, d, |
| | ArH), 7.32 (1 H, m, ArH), 7.19 (1 H, s, ArH), 6.11 (1 H, s, CH), 5.91 (1 H, d, ArH), |
| | 4.42 (1 H, s, NH), 4.06-4.03 (2 H, m, CH), 3.67 (2 H, m, CH), 3.56 (3 H, s, CH₃), |
| | 3.45 (3 H, s, CH₃), 3.38-3.30 (2 H, m, CH), 2.63 (2 H, d, CH), 2.54 (2 H, q, CH₂), |
| | 1.18 (6 H, t, CH₃). |
| 173 | ¹H NMR δ (ppm)(CHCl₃-d): 8.71 (1 H, s, ArH), 7.62 (1 H, s, ArH), 7.17 (1 H, d, |
| | ArH), 7.10 (1 H, s, ArH), 7.03-6.95 (2 H, m, ArH), 6.93 (1 H, s, ArH), 5.89 (1 H, s, |
| | ArH), 5.08 (2 H, s, CH₂), 3.58 (3 H, s, CH₃), 3.42 (3 H, s, CH₃), 2.50 (2 H, q, CH₂), |
| | 2.41 (3 H, s, CH₃), 1.11 (3 H, t, CH₃). |
| 174 | ¹H NMR δ (ppm)(CHCl₃-d): 8.72 (1 H, s, ArH), 7.85 (1 H, s, ArH), 7.69 (1 H, d, |
| | ArH), 7.63 (1 H, s, ArH), 7.26 (1 H, d, ArH), 6.76 (1 H, d, NH), 5.92 (1 H, s, ArH), |
| | 5.00 (1 H, m, CH), 4.07 (2 H, m, CH), 3.64-3.56 (2 H, m, CH), 3.53 (3 H, s, CH₃), |
| | 3.49 (3 H, s, CH₃), 2.56 (2 H, q, CH₂), 1.80 (1 H, s, NH), 1.19 (3 H, t, CH₃). |
| 175 | ¹H NMR δ (ppm)(CHCl₃-d): 8.69 (1 H, s, ArH), 7.83 (1 H, s, ArH), 7.71-7.66 (2 H, |
| | m, ArH), 7.27 (1 H, d, ArH), 6.95 (1 H, d, NH), 5.96 (1 H, s, ArH), 4.97-4.86 (1 H, |
| | m, CH), 4.23 (2 H, m, CH), 4.09 (2 H, dd, CH), 3.59 (3 H, s, CH₃), 3.45 (3 H, s, |
| | CH₃), 2.95 (3 H, s, CH₃), 2.55 (2 H, q, CH₂), 1.17 (3 H, t, CH₃). |
| 176 | ¹H NMR δ (ppm)(CHCl₃-d): 8.72 (1 H, d, ArH), 7.62 (1 H, s, ArH), 7.39 (1 H, d, |
| | ArH), 7.36-7.25 (1 H, m, ArH), 7.19 (1 H, d, ArH), 6.16-6.13 (1 H, m, CH), 5.92 (1 |
| | H, d, ArH), 4.01 (2 H, m, CH), 3.59-3.52 (5 H, m, CH₃, CH), 3.44 (3 H, s, CH₃), |
| | 2.88 (3 H, s, CH₃), 2.75-2.71 (2 H, m, CH), 2.54 (2 H, q, CH₂), 1.17 (3 H, t, CH₃). |
| 177 | ¹H NMR δ (ppm)(DMSO-d₆): 8.49 (1 H, d, ArH), 8.03 (1 H, s, ArH), 7.96 (1 H, d, |
| | ArH), 7.89 (1 H, dd, ArH), 7.40 (1 H, d, ArH), 6.39 (1 H, d, ArH), 3.65 (3 H, s, CH₃), |
| | 3.39 (3 H, s, CH₃), 2.61 (3 H, s, CH₃), 2.56-2.48 (2 H, m, CH₂), 1.14 (3 H, t, CH₃). |
| 178 | ¹H NMR δ (ppm)(DMSO-d₆): 8.49 (1 H, d, ArH), 8.08-7.98 (3 H, m, ArH), 7.43 (1 |
| | H, d, ArH), 6.47 (1 H, d, ArH), 3.67 (3 H, s, CH₃), 3.39 (3 H, s, CH₃), 2.58 (2 H, m, |
| | CH₂), 2.44 (3 H, s, CH₃), 1.14 (3 H, t, CH₃). |
| 179 | ¹H NMR δ (ppm)(DMSO-d₆): 8.51 (1 H, d, ArH), 8.19 (1 H, s, ArH), 7.94 (1 H, d, |
| | ArH), 7.85 (1 H, dd, ArH), 7.33 (1 H, d, ArH), 6.55 (1 H, s, ArH), 3.70 (3 H, s, CH₃), |
| | 3.39 (3 H, s, CH₃), 2.54-2.44 (2 H, m, CH), 1.12 (3 H, t, CH₃), (NH not observed). |
| 180 | ¹H NMR δ (ppm)(CHCl₃-d): 8.72 (1 H, d, ArH), 7.60 (1 H, s, ArH), 7.20 (1 H, s, |
| | ArH), 7.14-7.11 (2 H, m, ArH), 5.87 (1 H, d, ArH), 4.28 (2 H, s, CH), 3.56 (3 H, s, |
| | CH₃), 3.43 (3 H, s, CH₃), 2.69 (2 H, m, CH), 2.64 (1 H, m, CH), 2.51 (2 H, q, CH₂), |
| | 1.90 (2 H, d, CH), 1.67 (2 H, s, CH), 1.50 (9 H, s, CH₃), 1.15 (3 H, t, CH₃). |
| 181 | ¹H NMR δ (ppm)(CHCl₃-d): 8.72 (1 H, s, ArH), 7.60 (1 H, s, ArH), 7.34 (1 H, s, |
| | ArH), 7.24 (1 H, m, ArH), 7.17 (1 H, d, ArH), 5.88 (1 H, s, ArH), 3.94 (2 H, s, CH₂), |
| | 3.55 (3 H, s, CH₃), 3.44 (3 H, s, CH₃), 2.53 (2 H, q, CH₂), 1.62 (2 H, s, NH2), 1.16 (3 |
| | H, t, CH₃). |
| 182 | ¹H NMR δ (ppm)(CHCl₃-d): 8.70 (1 H, s, ArH), 7.61 (1 H, s, ArH), 7.30-7.13 (3 H, |
| | m, ArH), 5.91 (1 H, s, ArH), 5.89 (1 H, s, NH), 4.49 (2 H, d, CH₂), 3.58 (3 H, s, CH₃), |
| | 3.42 (3 H, s, CH₃), 2.52 (2 H, q, CH₂), 2.08 (3 H, s, CH₃), 1.15 (3 H, t, CH₃). |
| 183 | ¹H NMR δ (ppm)(DMSO-d₆): 13.60 (1 H, s, NH), 8.50 (1 H, s, ArH), 8.00 (2 H, m, |
| | ArH), 7.89 (1 H, dd, ArH), 7.26 (1 H, d, ArH), 6.21 (1 H, s, ArH), 3.60 (3 H, s, CH₃), |
| | 3.37 (3 H, s, CH₃), 2.57-2.45 (2 H, m, CH₂), 2.41 (3 H, s, CH₃), 1.14 (3 H, t, CH₃). |
| 184 | ¹H NMR δ (ppm)(DMSO-d₆): 8.80 (2 H, s, ArH), 8.49 (1 H, d, ArH), 8.03-7.96 (1 H, |
| | m, ArH), 7.66 (1 H, d, ArH), 7.57 (1 H, dd, ArH), 7.25 (1 H, d, ArH), 6.26 (1 H, s, |
| | ArH), 3.79-3.75 (4 H, m, CH), 3.72-3.68 (4 H, m, CH), 3.62 (3 H, s, CH₃), 3.36 (3 H, |
| | s, CH₃), 2.55-2.44 (2 H, m, CH₂), 1.20-1.09 (3 H, m, CH₃). |
| 185 | ¹H NMR δ (ppm)(DMSO-d₆): 12.90 (1 H, s, OH), 8.49 (1 H, d, ArH), 8.02 (1 H, s, |
| | ArH), 7.95 (1 H, d, ArH), 7.86 (1 H, dd, ArH), 7.30 (1 H, d, ArH), 6.33 (1 H, s, ArH), |
| | 3.64 (3 H, s, CH₃), 3.42-3.28 (3 H, m, CH₃), 2.53-2.44 (2 H, m, CH₂), 1.10 (3 H, t, |
| | CH₃). |
| 186 | ¹H NMR δ (ppm)(CHCl₃-d): 8.72 (1 H, s, ArH), 7.60 (1 H, s, ArH), 7.18-7.12 (3 H, |
| | m, ArH), 5.88 (1 H, d, ArH), 4.15-4.09 (2 H, m, CH), 3.63-3.48 (5 H, m, CH₃, CH), |
| | 3.44 (3 H, s, CH₃), 2.85-2.76 (1 H, m, CH), 2.52 (2 H, q, CH₂), 1.90-1.81 (4 H, m, |
| | CH), 1.16 (3 H, t, CH₃). |
| 187 | ¹H NMR δ (ppm)(CHCl₃-d): 8.72 (1 H, d, ArH), 8.65 (1 H, s, ArH), 7.62 (1 H, s, |
| | ArH), 7.23 (1 H, s, ArH), 7.16 (2 H, d, ArH), 5.88 (1 H, d, NH), 3.63-3.46 (5 H, m, |
| | CH₃, CH), 3.43 (3 H, s, CH₃), 3.03-2.92 (2 H, m, CH), 2.85-2.73 (2 H, m, CH), |
| | 2.66 (1 H, s, CH), 2.57-2.47 (2 H, m, CH₂), 2.12-2.04 (2 H, m, CH), 1.15 (3 H, t, CH₃). |
| 188 | ¹H NMR δ (ppm)(CHCl₃-d): 8.73 (1 H, d, ArH), 8.04 (1 H, s, ArH), 7.93 (1 H, s, |
| | ArH), 7.62 (1 H, s, ArH), 7.27 (1 H, s, ArH), 5.90 (1 H, s, ArH), 3.51 (6 H, m, CH₃), |
| | 2.58 (2 H, q, CH₂), 2.09-2.02 (1 H, m, CH), 1.23-1.08 (7 H, m, CH₃, CH), (NH not |
| | observed). |
| 189 | ¹H NMR δ (ppm)(CHCl₃-d): 9.09 (1 H, s, ArH), 8.87 (1 H, s, ArH), 8.74 (1 H, d, |
| | ArH), 8.19 (1 H, s, ArH), 7.65 (1 H, s, ArH), 7.58 (1 H, s, ArH), 7.52 (1 H, m, ArH), |
| | 7.37 (1 H, d, ArH), 6.03 (1 H, d, ArH), 3.62 (3 H, s, CH₃), 3.48 (3 H, s, CH₃), 2.62 (2 |
| | H, q, CH₂), 1.23 (3 H, t, CH₃). |
| 190 | ¹H NMR δ (ppm)(CHCl₃-d): 8.70 (1 H, s, ArH), 7.62 (1 H, s, ArH), 7.37 (1 H, s, |
| | ArH), 7.31 (2 H, m, ArH), 5.90 (1 H, s, ArH), 4.69 (1 H, t, NH), 4.38 (2 H, d, CH₂), |
| | 3.58 (3 H, s, CH₃), 3.43 (3 H, s, CH₃), 3.00 (3 H, s, CH₃), 2.53 (2 H, q, CH₂), 1.16 (3 |
| | H, t, CH₃). |
| 191 | ¹H NMR δ (ppm)(CHCl₃-d): 8.70 (1 H, s, ArH), 7.61 (1 H, s, ArH), 7.28 (1 H, s, |
| | ArH), 7.20 (2 H, m, ArH), 5.89 (1 H, s, ArH), 5.15 (1 H, s, NH), 4.43 (2 H, d, CH₂), |
| | 3.74 (3 H, s, CH₃), 3.57 (3 H, s, CH₃), 3.42 (3 H, s, CH₃), 2.51 (2 H, q, CH₂), 1.15 (3 |
| | H, t, CH₃). |
| 192 | ¹H NMR δ (ppm)(CHCl₃-d): 8.66 (1 H, s, ArH), 7.61 (1 H, s, ArH), 7.27 (1 H, s, |
| | ArH), 7.21 (1 H, d, ArH), 7.12 (1 H, d, ArH), 5.94 (1 H, s, ArH), 5.03 (1 H, t, NH), |
| | 4.72 (1 H, t, NH), 4.40 (2 H, d, CH₂), 3.58 (3 H, s, CH₃), 3.40 (3 H, s, CH₃), 3.25 (2 |
| | H, m, CH₂), 2.49 (2 H, q, CH₂), 1.17-1.08 (6 H, m, CH₃). |
| 193 | ¹H NMR δ (ppm)(CHCl₃-d): 8.74 (1 H, d, ArH), 7.88 (1 H, d, ArH), 7.70 (1 H, m, |
| | ArH), 7.63 (1 H, s, ArH), 7.27 (1 H, s, ArH), 5.96 (1 H, d, ArH), 5.17 (2 H, s, NH2), |
| | 3.58 (3 H, s, CH₃), 3.47 (3 H, s, CH₃), 2.58 (2 H, q, CH₂), 1.20 (3 H, t, CH₃). |
| 194 | ¹H NMR δ (ppm)(DMSO-d₆): 9.27 (1 H, d, ArH), 8.96 (1 H, d, ArH), 8.52 (1 H, s, |
| | ArH), 8.48 (1 H, d, ArH), 7.99 (1 H, s, ArH), 7.86 (1 H, d, ArH), 7.82-7.73 (1 H, m, |
| | ArH), 7.35-7.27 (1 H, m, ArH), 6.31 (1 H, s, ArH), 3.62 (3 H, s, CH₃), 3.37 (3 H, s, |
| | CH₃), 2.56-2.49 (2 H, m, CH₂), 1.23-1.11 (3 H, m, CH₃). |
| 195 | ¹H NMR δ (ppm)(DMSO-d₆): 8.51 (1 H, s, ArH), 8.14 (1 H, s, ArH), 7.48 (1 H, dd, |
| | ArH), 7.43-7.37 (1 H, m, ArH), 7.20 (1 H, d, ArH), 6.36 (1 H, s, CH), 6.23 (1 H, d, |
| | ArH), 4.16 (2 H, s, CH), 4.10 (2 H, s, CH), 3.74-3.51 (5 H, m, CH, CH₃), 3.36 (3 H, s, |
| | CH₃), 2.66-2.42 (4 H, m, CH, CH₂), 1.11 (3 H, t, CH₃). |
| 196 | ¹H NMR δ (ppm)(DMSO-d₆): 8.51 (1 H, d, ArH), 8.20-8.08 (1 H, m, ArH), 7.48 (1 |
| | H, dd, ArH), 7.43-7.37 (1 H, m, ArH), 7.19 (1 H, d, ArH), 6.32 (1 H, s, CH), 6.24 (1 |
| | H, d, ArH), 4.16 (2 H, s, CH), 3.76-3.61 (5 H, m, CH, CH₃), 3.36 (3 H, s, CH₃), |
| | 2.81 (2 H, m, CH), 2.73-2.59 (4 H, m, CH), 2.46 (2 H, m, CH₂), 1.19-1.07 (3 H, m, CH₃). |
| 197 | ¹H NMR δ (ppm)(DMSO-d₆): 8.48 (1 H, m, ArH), 8.02-7.95 (1 H, m, ArH), 7.46 (1 |
| | H, dd, ArH), 7.43-7.35 (1 H, m, ArH), 7.20-7.13 (1 H, m, ArH), 6.85 and 6.79 (1 H, 2 |
| | t, CH), 6.26-6.20 (1 H, m, CH), 6.16 (1 H, s, ArH), 4.26 and 4.20 (2 H, 2 s, CH), |
| | 3.80-3.73 (2 H, m, CH), 3.59 (3 H, s, CH₃), 3.32 (3 H, s, CH₃), 3.00-2.89 (1 H, m, |
| | CH), 2.68-2.53 (1 H, m, CH), 2.46 (2 H, q, CH₂), 1.10 (3 H, t, CH₃). |
| 198 | ¹H NMR δ (ppm)(DMSO-d₆): 8.48 (1 H, dd, ArH), 8.01-7.97 (1 H, m, ArH), 7.48 (1 |
| | H, d, ArH), 7.40 (1 H, dd, ArH), 7.17 (1 H, d, ArH), 6.29 (1 H, s, CH), 6.15 (1 H, s, |
| | ArH), 4.56-3.88 (4 H, m, CH), 3.59 (3 H, s, CH₃), 3.33 (3 H, s, CH₃), 3.02-2.64 (2 H, |
| | m, CH), 2.46 (2 H, q, CH₂), 1.68-1.63 (2 H, m, CH), 1.54 (2 H, s, CH), 1.11 (3 H, t, |
| | CH₃). |
| 199 | ¹H NMR δ (ppm)(DMSO-d₆): 8.49 (1 H, d, ArH), 7.98 (1 H, s, ArH), 7.45 (1 H, s, |
| | ArH), 7.37 (1 H, d, ArH), 7.16 (1 H, d, ArH), 6.29-6.15 (1 H, m, CH), 6.15 (1 H, s, |
| | ArH), 4.20-4.07 (4 H, m, CH), 3.73-3.53 (5 H, m, CH, CH₃), 3.33 (3 H, s, CH₃), |
| | 2.59 (2 H, s, CH), 2.54-2.42 (2 H, m, CH₂), 1.15-1.06 (3 H, m, CH₃), (CH₃ under water |
| | peak). |
| 200 | ¹H NMR δ (ppm)(DMSO-d₆): 8.49 (1 H, d, ArH), 7.98 (1 H, s, ArH), 7.45 (1 H, d, |
| | ArH), 7.40-7.35 (1 H, m, ArH), 7.16 (1 H, d, ArH), 6.27-6.18 (1 H, m, CH), 6.15 (1 |
| | H, s, ArH), 4.87 (1 H, s, OH), 4.28-4.15 (2 H, m, CH), 3.76-3.71 (2 H, m, CH), |
| | 3.59 (3 H, s, CH₃), 3.34 (3 H, s, CH₃), 2.62-2.38 (6 H, m, CH, CH₂), 1.25-1.07 (9 H, m, |
| | CH₃). |
| 201 | ¹H NMR δ (ppm)(DMSO-d₆): 8.51 (1 H, d, ArH), 8.19-8.11 (1 H, m, ArH), 7.49 (1 |
| | H, s, ArH), 7.41 (1 H, d, ArH), 7.20 (1 H, d, ArH), 6.39 (1 H, s, CH), 6.26 (1 H, s, |
| | ArH), 4.95 (1 H, s, OH), 4.56-4.10 (3 H, m, CH), 3.88-3.72 (2 H, m, CH), 3.65 (3 H, |
| | s, CH₃), 3.36 (3 H, s, CH₃), 2.60 (2 H, s, CH), 2.56-2.42 (2 H, m, CH₂), 1.27-1.18 (3 |
| | H, m, CH₃), 1.11 (3 H, t, CH₃). |
| 202 | ¹H NMR δ (ppm)(DMSO-d₆): 8.48 (1 H, d, ArH), 7.98 (1 H, s, ArH), 7.44 (1 H, d, |
| | ArH), 7.36 (1 H, dd, ArH), 7.14 (1 H, d, ArH), 6.79 (1 H, s, NH), 6.20 (1 H, s, CH), |
| | 6.15 (1 H, s, ArH), 3.94 (2 H, d, CH), 3.59 (3 H, s, CH₃), 3.53-3.47 (2 H, m, CH), |
| | 3.33 (3 H, s, CH₃), 2.54-2.42 (4 H, m, CH, CH₂), 1.29 (3 H, s, CH₃), 1.10 (3 H, t, |
| | CH₃), 0.64-0.59 (2 H, m, CH), 0.51-0.46 (2 H, m, CH). |
| 203 | ¹H NMR δ (ppm)(DMSO-d₆): 8.49 (1 H, d, ArH), 7.99 (1 H, s, ArH), 7.45 (1 H, d, |
| | ArH), 7.37 (1 H, dd, ArH), 7.15 (1 H, d, ArH), 6.21 (1 H, s, CH), 6.16 (1 H, s, ArH), |
| | 4.06 (2 H, d, CH), 3.65-3.55 (5 H, m, CH, CH₃), 3.33 (3 H, d, CH₃), 2.54-2.43 (4 H, |
| | m, CH, CH₂), 1.10 (3 H, t, CH₃), (NH not observed). |
| 204 | ¹H NMR δ (ppm)(DMSO-d₆): 8.49 (1 H, d, ArH), 7.98 (1 H, s, ArH), 7.45 (1 H, d, |
| | ArH), 7.37 (1 H, dd, ArH), 7.15 (1 H, d, ArH), 6.46 (1 H, m, NH), 6.23 (1 H, s, CH), |
| | 6.14 (1 H, s, ArH), 3.98 (2 H, d, CH), 3.66-3.50 (5 H, m, CH, CH₃), 3.34 (3 H, s, |
| | CH₃), 2.61 (3 H, d, CH₃), 2.57-2.41 (4 H, m, CH, CH₂), 1.10 (3 H, t, CH₃). |
| 205 | ¹H NMR δ (ppm)(DMSO-d₆): 8.48 (1 H, d, ArH), 7.98 (1 H, s, ArH), 7.44 (1 H, d, |
| | ArH), 7.36 (1 H, dd, ArH), 7.15 (1 H, d, ArH), 6.20 (1 H, s, CH), 6.14 (1 H, s, ArH), |
| | 3.97-3.90 (6 H, m, CH), 3.59 (3 H, s, CH₃), 3.49-3.43 (2 H, m, CH), 3.33 (3 H, s, |
| | CH₃), 2.54-2.42 (4 H, m, CH), 2.23-2.13 (2 H, m, CH₂), 1.10 (3 H, t, CH₃). |
| 206 | ¹H NMR δ (ppm)(DMSO-d₆): 8.49 (1 H, d, ArH), 7.98 (1 H, s, ArH), 7.45 (1 H, d, |
| | ArH), 7.38 (1 H, dd, ArH), 7.15 (1 H, d, ArH), 6.23 (1 H, s, CH), 6.13 (1 H, s, ArH), |
| | 3.85 (2 H, d, CH), 3.58 (3 H, s, CH₃), 3.40-3.31 (5 H, m, CH, CH₃), 3.16 (2 H, q, |
| | CH₂), 2.77 (3 H, s, CH₃), 2.56 (2 H, s, CH), 2.54-2.42 (2 H, m, CH₂), 1.12-1.07 (6 H, |
| | m, CH₃). |
| 207 | ¹H NMR δ (ppm)(DMSO-d₆): 8.49 (1 H, d, ArH), 7.98 (1 H, s, ArH), 7.45 (1 H, d, |
| | ArH), 7.38 (1 H, dd, ArH), 7.16 (1 H, d, ArH), 6.23 (1 H, s, CH), 6.14 (1 H, s, ArH), |
| | 4.14 (2 H, s, CH₂), 3.96 (2 H, d, CH), 3.59 (3 H, s, CH₃), 3.49-3.42 (2 H, m, CH₂), |
| | 3.33 (3 H, s, CH₃), 2.90 (3 H, s, CH₃), 2.63-2.37 (4 H, m, CH, CH₂), 1.10 (3 H, t, |
| | CH₃). |
| 208 | ¹H NMR δ (ppm)(DMSO-d₆): 8.50 (1 H, d, ArH), 8.28 (1 H, s, ArH), 8.01-7.88 (2 H, |
| | m, ArH), 7.63-7.57 (1 H, m, ArH), 7.49 (1 H, dd, ArH), 7.30-7.22 (2 H, m, ArH), |
| | 7.15 (1 H, d, ArH), 6.95-6.90 (2 H, m, ArH), 6.11 (1 H, s, ArH), 5.28 (2 H, s, CH₂), |
| | 3.74 (3 H, s, CH₃), 3.57 (3 H, s, CH₃), 3.34 (3 H, d, CH₃), 2.49-2.42 (2 H, m, CH₂), |
| | 1.12 (3 H, t, CH₃). |
| 209 | ¹H NMR δ (ppm)(CHCl₃-d): 8.73 (1 H, d, ArH), 7.91-7.89 (1 H, m, ArH), 7.83 (1 H, |
| | s, ArH), 7.62 (1 H, s, ArH), 7.53-7.47 (1 H, m, ArH), 7.42 (1 H, dd, ArH), 7.23 (1 H, |
| | m, ArH), 5.94 (1 H, d, ArH), 5.14 (2 H, s, CH₂), 3.57 (3 H, s, CH₃), 3.47 (3 H, s, |
| | CH₃), 2.57 (2 H, q, CH₂), 1.20 (3 H, t, CH₃). |
| 210 | ¹H NMR δ (ppm)(DMSO-d₆): 12.95 (1 H, s, NH), 8.50 (1 H, d, ArH), 8.22 (1 H, s, |
| | ArH), 7.97 (2 H, s, ArH), 7.63 (1 H, d, ArH), 7.53 (1 H, dd, ArH), 7.16 (1 H, d, ArH), |
| | 6.13 (1 H, d, ArH), 3.58 (3 H, s, CH₃), 3.34 (3 H, d, CH₃), 2.54-2.43 (2 H, m, CH₂), |
| | 1.13 (3 H, t, CH₃). |
| 211 | ¹H NMR δ (ppm)(CHCl₃-d): 8.74 (1 H, d, ArH), 8.60 (2 H, s, ArH), 7.62 (1 H, s, |
| | ArH), 7.48 (1 H, d, ArH), 7.41 (1 H, m, ArH), 7.31 (1 H, m, ArH), 5.97 (1 H, d, |
| | ArH), 5.20 (1 H, s, NH), 3.59 (3 H, s, CH₃), 3.47 (3 H, s, CH₃), 3.08 (3 H, d, CH₃), |
| | 2.59 (2 H, q, CH₂), 1.20 (3 H, t, CH₃). |
| 212 | ¹H NMR δ (ppm)(CHCl₃-d): 8.78-8.70 (3 H, m, ArH), 7.64 (1 H, s, ArH), 7.52 (1 H, |
| | d, ArH), 7.45 (1 H, m, ArH), 7.33 (1 H, d, ArH), 6.00 (1 H, d, ArH), 5.72-5.66 (1 H, |
| | m, CH), 5.03 (2 H, m, CH), 4.85 (2 H, m, CH), 3.61 (3 H, s, CH₃), 3.47 (3 H, s, CH₃), |
| | 2.60 (2 H, q, CH₂), 1.21 (3 H, t, CH₃). |
| 213 | ¹H NMR δ (ppm)(CHCl₃-d): 8.72 (1 H, d, ArH), 7.60 (1 H, s, ArH), 7.20 (1 H, s, |
| | ArH), 7.19-7.08 (2 H, m, ArH), 5.87 (1 H, d, ArH), 4.45 (1 H, m, NH), 4.11 (2 H, d, |
| | CH), 3.56 (3 H, s, CH₃), 3.49-3.39 (3 H, m, CH₃), 3.32 (2 H, m, CH), 2.90 (2 H, m, |
| | CH), 2.72 (1 H, m, CH), 2.51 (2 H, q, CH₂), 1.93 (2 H, d, CH₂), 1.77-1.64 (2 H, m, |
| | CH), 1.24-1.10 (6 H, m, CH₃). |
| 214 | ¹H NMR δ (ppm)(CHCl₃-d): 8.72 (1 H, d, ArH), 7.61 (1 H, s, ArH), 7.21 (1 H, s, |
| | ArH), 7.14 (2 H, d, ArH), 5.89 (1 H, d, ArH), 4.00 (2 H, m, CH), 3.57 (3 H, s, CH₃), |
| | 3.43 (3 H, s, CH₃), 2.84 (3 H, s, CH₃), 2.80 (2 H, d, CH), 2.52 (1 H, m, CH), 2.49 (2 |
| | H, q, CH₂), 2.02 (2 H, m, CH), 1.92 (2 H, m, CH), 1.15 (3 H, t, CH₃). |
| 215 | ¹H-NMR δ (ppm)(DMSO-d₆): 8.50-8.48 (1 H, m, ArH), 7.98 (1 H, s, ArH), 7.46 (1 H, |
| | d, ArH), 7.38 (1 H, dd, ArH), 7.15 (2 H, m, ArH, NH), 6.23 (1 H, s, CH), 6.15 (1 H, |
| | s, ArH), 4.78-4.65 (3 H, m, CH), 4.50 (2 H, m, CH), 4.03 (2 H, d, CH), 3.64-3.53 (5 |
| | H, m, CH, CH₃), 3.37-3.28 (3 H, s, CH₃), 2.54-2.42 (4 H, m, CH, CH₂), 1.10 (3 H, t, |
| | CH₃). |
| 216 | ¹H NMR δ (ppm)(DMSO-d₆): 8.49 (1 H, d, ArH), 7.98 (1 H, s, ArH), 7.45 (1 H, d, |
| | ArH), 7.37 (1 H, dd, ArH), 7.16 (1 H, ArH), 6.22 (1 H, d, CH), 6.15 (1 H, s, ArH), |
| | 4.99 (2 H, s, CH₂), 4.12 (2 H, s, CH), 3.71-3.56 (5 H, m, CH, CH₃), 3.34 (3 H, s, |
| | CH₃), 2.61-2.38 (4 H, m, CH, CH₂), 1.16-1.06 (3 H, m, CH₃). |
| 217 | ¹H NMR δ (ppm)(CHCl₃-d): 8.85 (2 H, s, ArH), 8.74 (1 H, d, ArH), 7.65 (1 H, s, |
| | ArH), 7.36 (1 H, s, ArH), 7.34 (1 H, m, ArH), 7.26 (1 H, m, ArH), 6.01 (1 H, d, ArH), |
| | 5.13 (2 H, s, CH₂), 3.61 (3 H, s, CH₃), 3.48 (3 H, s, CH₃), 2.54 (2 H, m, CH₂), 1.24 (3 |
| | H, s, CH₃). |
| 218 | ¹H NMR δ (ppm)(DMSO-d₆): 9.29 (1 H, d, ArH), 9.04 (1 H, d, ArH), 8.58 (1 H, t, |
| | ArH), 8.48 (1 H, d, ArH), 7.99 (1 H, s, ArH), 7.85 (1 H, d, ArH), 7.77 (1 H, dd, ArH), |
| | 7.35 (1 H, d, ArH), 6.32 (1 H, s, ArH), 3.62 (3 H, s, CH₃), 3.42 (3 H, s, CH₃), 3.37 (3 |
| | H, s, CH₃), 2.58-2.49 (2 H, m, CH₂), 1.24-1.12 (3 H, m, CH₃). |
| 219 | ¹H NMR δ (ppm)(CHCl₃-d): 8.74 (1 H, dd, ArH), 7.86-7.76 (2 H, m, ArH), |
| | 7.65-7.52 (1 H, m, ArH), 7.54-7.48 (1 H, m, ArH), 7.42 (1 H, dd, ArH), 7.20 (1 H, d, ArH), |
| | 5.93 (1 H, d, ArH), 4.36 (2 H, t, CH₂), 3.67-3.42 (3 H, m, CH₃), 3.55-3.40 (3 H, m, |
| | CH₃), 3.06 (2 H, t, CH₂), 2.65-2.49 (6 H, m, CH), 2.10-1.56 (4 H, m, CH), |
| | 1.28-1.13 (3 H, m, CH₃). |
| 220 | ¹H NMR δ (ppm)(CHCl₃-d): 8.73 (1 H, d, ArH), 7.85 (2 H, s, ArH), 7.67-7.60 (1 H, |
| | m, ArH), 7.52-7.49 (1 H, m, ArH), 7.43 (1 H, dd, ArH), 7.19 (1 H, d, ArH), 5.89 (1 |
| | H, d, ArH), 5.05 (2 H, s, CH₂), 3.69-3.43 (3 H, m, CH₃), 3.47 (3 H, s, CH₃), 3.13 (3 |
| | H, s, CH₃), 3.02 (3 H, s, CH₃), 2.54 (2 H, q, CH₂), 1.23-1.14 (3 H, m, CH₃). |
| 221 | ¹H NMR δ (ppm)(CHCl₃-d): 8.74 (1 H, dd, ArH), 7.86 (1 H, d, ArH), 7.72 (1 H, d, |
| | ArH), 7.60 (1 H, s, ArH), 7.53-7.50 (1 H, m, ArH), 7.43 (1 H, dd, ArH), 7.19 (1 H, d, |
| | ArH), 5.88 (1 H, m, ArH), 5.19 (2 H, s, CH₂), 3.71-3.41 (3 H, m, CH₃), 3.47 (3 H, s, |
| | CH₃), 2.55 (2 H, q, CH₂), 1.30 (9 H, s, CH₃), 1.18 (3 H, t, CH₃). |
| 222 | ¹H NMR δ (ppm)(CHCl₃-d): 8.75-8.72 (1 H, m, ArH), 7.94 (1 H, d, ArH), 7.78 (1 H, |
| | d, ArH), 7.62 (1 H, s, ArH), 7.53-7.49 (1 H, m, ArH), 7.43 (1 H, dd, ArH), |
| | 7.29-7.18 (1 H, m, ArH), 6.26 (1 H, s, NH), 5.94 (1 H, d, ArH), 5.53 (1 H, s, NH), 4.87 (2 H, s, |
| | CH₂), 3.57 (3 H, s, CH₃), 3.46 (3 H, s, CH₃), 2.56 (2 H, q, CH₂), 1.19 (3 H, t, CH₃). |
| 223 | ¹H NMR δ (ppm)(CHCl₃-d): 8.72 (1 H, s, ArH), 7.61 (1 H, s, ArH), 7.20 (1 H, s, |
| | ArH), 7.16-7.10 (2 H, m, ArH), 5.88 (1 H, s, ArH), 4.84 and 4.01 (2 H, 2 d, CH), |
| | 3.57 (3 H, s, CH₃), 3.43 (3 H, s, CH₃), 3.16 and 2.79 (2 H, 2 m, CH), 2.66 (1 H, s, CH), |
| | 2.51 (2 H, q, CH₂), 2.41 (2 H, q, CH₂), 1.69 (2 H, m, CH), 1.97 (2 H, m, CH₂), 1.17 (6 |
| | H, m, CH₃), (rotamers observed). |
| 224 | ¹H NMR δ (ppm)(CHCl₃-d): 8.71 (1 H, d, ArH), 7.66 (1 H, s, ArH), 7.16 (1 H, s, |
| | ArH), 7.15-7.11 (2 H, m, ArH), 5.92 (1 H, s, ArH), 4.76 and 3.88 (2 H, 2 m, CH), |
| | 3.59 (3 H, s, CH₃), 3.54 (2 H, d, CH), 3.44 (3 H, s, CH₃), 2.84 (1 H, m, CH), 2.81 (2 |
| | H, m, CH), 2.51 (2 H, q, CH₂), 2.04 (2 H, m, CH), 2.00 (2 H, m, CH), 1.15 (3 H, t, |
| | CH₃), (rotamers observed). |
| 225 | ¹H NMR δ (ppm)(CHCl₃-d): 8.67 (1 H, s, ArH), 7.73 (1 H, s, ArH), 7.26 (1 H, s, |
| | ArH), 7.15 (2 H, d, ArH), 6.16 (1 H, t, CH), 6.02 (1 H, m, ArH), 4.71 and 4.26 (2 H, |
| | 2 d, CH), 3.62 (3 H, s, CH₃), 3.45 (3 H, s, CH₃), 3.23 (1 H, m, CH), 2.92-2.82 (2 H, |
| | m, CH), 2.51 (2 H, q, CH₂), 2.05 (2 H, d, CH), 1.79-1.70 (2 H, m, CH), 1.16 (3 H, t, |
| | CH₃), (rotamers observed). |
| 226 | ¹H NMR δ (ppm)(CHCl₃-d): 8.67 (1 H, s, ArH), 7.70 (1 H, s, ArH), 7.22 (1 H, s, |
| | ArH), 7.14 (2 H, d, ArH), 6.00 (1 H, s, ArH), 4.78 and 4.03 (2 H, 2 d, CH), 4.18 (2 H, |
| | m, CH), 3.61 (3 H, s, CH₃), 3.47 (6 H, m, CH₃), 3.15 and 2.71 (2 H, 2 m, CH), 2.81 (1 |
| | H, m, CH), 2.51 (2 H, q, CH₂), 2.11-1.89 (2 H, m, CH), 1.71 (2 H, m, CH), 1.16 (3 H, |
| | t, CH₃), (rotamers observed). |
| 227 | ¹H NMR δ (ppm)(DMSO-d₆): 8.48 (1 H, s, ArH), 7.98 (1 H, s, ArH), 7.68 (1 H, t, |
| | NH), 7.38 (1 H, s, ArH), 7.29 (1 H, dd, ArH), 7.16 (1 H, d, ArH), 6.12 (1 H, s, ArH), |
| | 4.23 (2 H, d, CH₂), 3.58 (3 H, s, CH₃), 3.33 (3 H, s, CH₃), 2.56-2.41 (3 H, m, CH, |
| | CH₂), 1.13-1.04 (3 H, m, CH₃), 0.94-0.89 (4 H, m, CH₂). |
| 228 | ¹H NMR δ (ppm)(CHCl₃-d): 8.70 (1 H, d, ArH), 7.63 (1 H, s, ArH), 7.39 (1 H, d, |
| | ArH), 7.33-7.24 (2 H, m, ArH), 5.93 (1 H, d, ArH), 5.28 (1 H, s, NH), 4.51 (2 H, s, |
| | CH), 3.97 (2 H, s, CH), 3.59 (3 H, s, CH₃), 3.44 (3 H, s, CH₃), 2.55 (2 H, q, CH₂), |
| | 1.17 (3 H, t, CH₃). |
| 229 | ¹H NMR δ (ppm)(CHCl₃-d): 8.72 (1 H, d, ArH), 7.62 (1 H, s, ArH), 7.41 (1 H, d, |
| | ArH), 7.35-7.31 (1 H, d, ArH), 7.17 (1 H, d, ArH), 6.19-6.14 (1 H, m, CH), 5.91 (1 H, |
| | s, ArH), 4.36 (2 H, s, CH), 3.98 (2 H, s, CH), 3.56 (3 H, s, CH₃), 3.45 (3 H, s, CH₃), |
| | 2.90 (1 H, s, OH), 2.68 (2 H, s, CH), 2.54 (2 H, q, CH), 1.23-1.15 (4 H, m, CH₂), |
| | 1.05-1.01 (3 H, m, CH₃). |
| 230 | ¹H NMR δ (ppm)(CHCl₃-d): 8.72 (1 H, d, ArH), 7.63 (1 H, s, ArH), 7.39 (1 H, s, |
| | ArH), 7.31 (1 H, dd, ArH), 7.19 (1 H, d, ArH), 6.16 and 6.09 (1 H, s, CH), 5.93 (1 H, |
| | s, ArH), 4.93-4.81 (1 H, m, CH), 4.46-4.21 (3 H, m, CH), 3.86-3.69 (2 H, m, CH), |
| | 3.57 (3 H, s, CH₃), 3.45 (3 H, s, CH₃), 2.70 (2 H, s, CH), 2.54 (2 H, q, CH), 1.17 (3 H, |
| | t, CH₃), (rotamers observed). |
| 231 | ¹H NMR δ (ppm)(CHCl₃-d): 8.72 (1 H, d, ArH), 7.62 (1 H, s, ArH), 7.40 (1 H, d, |
| | ArH), 7.33 (1 H, dd, ArH), 7.19 (1 H, d, ArH), 6.16 (1 H, s, CH), 5.92 (1 H, d, ArH), |
| | 4.39 (1 H, s, OH), 4.36 (2 H, s, CH), 3.94-3.88 (2 H, m, CH), 3.57 (3 H, s, CH₃), |
| | 3.44 (3 H, s, CH₃), 2.67 (2 H, s, CH), 2.54 (2 H, q, CH), 1.56 (6 H, s, CH₃), 1.17 (3 H, t, |
| | CH₃). |
| 232 | ¹H NMR δ (ppm)(CHCl₃-d): 8.72 (1 H, d, ArH), 7.63 (1 H, s, ArH), 7.38 (1 H, d, |
| | ArH), 7.31 (1 H, dd, ArH), 7.19 (1 H, d, ArH), 6.15-6.08 (1 H, m, CH), 5.92 (1 H, s, |
| | ArH), 4.11 (2 H, d, CH), 3.83-3.72 (2 H, m, CH), 3.69-3.63 (2 H, m, CH), 3.57 (3 H, |
| | s, CH₃), 3.45 (3 H, s, CH₃), 2.72 (2 H, s, CH), 2.54 (2 H, q, CH₂), 1.17 (3 H, t, CH₃). |
| 233 | ¹H NMR δ (ppm)(CHCl₃-d): 8.72 (1 H, d, ArH), 7.61 (1 H, s, ArH), 7.40 (1 H, d, |
| | ArH), 7.36-7.30 (1 H, m, ArH), 7.18 (1 H, d, ArH), 6.18-6.10 (1 H, m, CH), 5.90 (1 |
| | H, s, ArH), 3.97 (2 H, d, CH), 3.56 (3 H, s, CH₃), 3.54-3.50 (2 H, m, CH), 3.44 (3 H, |
| | s, CH₃), 3.27-3.22 (4 H, m, CH), 2.67 (2 H, d, CH), 2.53 (2 H, q, CH₂), 1.68-1.61 (4 |
| | H, m, CH), 1.61-1.54 (2 H, d, CH), 1.16 (3 H, t, CH₃). |
| 234 | ¹H NMR δ (ppm)(CHCl₃-d): 11.00 (1 H, s, NH), 8.73 (2 H, d, ArH), 7.62 (1 H, s, |
| | ArH), 7.42 (1 H, d, ArH), 7.34 (1 H, dd, ArH), 7.19 (1 H, d, ArH), 6.18 (1 H, s, CH), |
| | 5.92 (1 H, s, ArH), 4.44 (2 H, s, CH), 3.98-3.92 (2 H, m, CH), 3.57 (3 H, s, CH₃), |
| | 3.45 (3 H, s, CH₃), 2.74 (2 H, s, CH), 2.54 (2 H, q, CH₂), 1.17 (3 H, t, CH₃). |
| 235 | ¹H NMR δ (ppm)(CHCl₃-d): 8.71 (1 H, d, ArH), 7.64 (1 H, s, ArH), 7.20 (1 H, s, |
| | ArH), 7.14 (2 H, s, ArH), 5.90 (1 H, s, ArH), 4.84-4.49 (2 H, m, CH), 4.04-3.83 (3 H, |
| | m, CH, OH), 3.58 (3 H, s, CH₃), 3.43 (3 H, s, CH₃), 3.24-3.14 (1 H, m, CH), |
| | 2.89-2.74 (2 H, m, CH), 2.52 (2 H, q, CH₂), 2.02 (2 H, d, CH), 1.40 and 1.36 (3 H, 2 d, |
| | CH₃), 1.15 (3 H, t, CH₃), (rotamers observed). |
| 236 | ¹H NMR δ (ppm)(CHCl₃-d): 8.73 (1 H, d, ArH), 7.87 (1 H, s, ArH), 7.67 (1 H, s, |
| | ArH), 7.60 (1 H, s, ArH), 7.48 (1 H, d, ArH), 7.43-7.36 (1 H, m, ArH), 7.32-7.23 (1 |
| | H, m, ArH), 7.18 (1 H, d, ArH), 6.87 (2 H, dd, ArH), 6.81 (1 H, m, ArH), 5.90 (1 H, |
| | d, ArH), 5.34 (2 H, s, CH₂), 3.80 (3 H, s, CH₃), 3.54 (3 H, s, CH₃), 3.46 (3 H, s, CH₃), |
| | 2.54 (2 H, q, CH₂), 1.17 (3 H, t, CH₃). |
| 237 | ¹H NMR δ (ppm)(CHCl₃-d): 8.73 (1 H, d, ArH), 7.85 (1 H, s, ArH), 7.64 (1 H, s, |
| | ArH), 7.60 (1 H, s, ArH), 7.48-7.43 (1 H, m, ArH), 7.39 (1 H, dd, ArH), 7.21-7.13 (5 |
| | H, m, ArH), 5.90 (1 H, d, ArH), 5.32 (2 H, s, CH₂), 3.54 (3 H, s, CH₃), 3.45 (3 H, s, |
| | CH₃), 2.53 (2 H, q, CH₂), 2.46-2.27 (3 H, m, CH₃), 1.17 (3 H, t, CH₃). |
| 238 | ¹H NMR δ (ppm)(CHCl₃-d): 8.73 (1 H, d, ArH), 7.87 (1 H, s, ArH), 7.66 (1 H, s, |
| | ArH), 7.60 (1 H, s, ArH), 7.47 (1 H, m, ArH), 7.40 (1 H, dd, ArH), 7.29-7.22 (2 H, m, |
| | ArH), 7.19 (1 H, d, ArH), 7.09-7.02 (2 H, m, ArH), 5.91 (1 H, d, ArH), 5.33 (2 H, s, |
| | CH), 3.54 (3 H, s, CH₃), 3.45 (3 H, s, CH₃), 2.54 (2 H, q, CH), 1.17 (3 H, t, CH₃). |
| 239 | ¹H NMR δ (ppm)(CHCl₃-d): 8.73 (1 H, d, ArH), 7.87 (1 H, s, ArH), 7.67 (1 H, s, |
| | ArH), 7.60 (1 H, s, ArH), 7.48 (1 H, d, ArH), 7.40 (1 H, dd, ArH), 7.36-7.32 (2 H, m, |
| | ArH), 7.23-7.15 (3 H, m, ArH), 5.91 (1 H, d, ArH), 5.33 (2 H, s, CH), 3.54 (3 H, s, |
| | CH₃), 3.46 (3 H, s, CH₃), 2.54 (2 H, q, CH₂), 1.18 (3 H, t, CH₃). |
| 240 | ¹H NMR δ (ppm)(CHCl₃-d): 8.73 (1 H, d, ArH), 7.82 (1 H, s, ArH), 7.68 (1 H, s, |
| | ArH), 7.60 (1 H, s, ArH), 7.50 (1 H, d, ArH), 7.42 (1 H, dd, ArH), 7.20 (1 H, d, ArH), |
| | 5.92 (1 H, d, ArH), 4.14 (2 H, t, CH₂), 3.55 (3 H, s, CH₃), 3.46 (3 H, s, CH₃), 2.55 (2 |
| | H, q, CH₂), 2.01-1.89 (2 H, m, CH₂), 1.19 (3 H, t, CH₃), 0.97 (3 H, t, CH₃). |
| 241 | ¹H NMR δ (ppm)(CHCl₃-d): 8.73 (1 H, d, ArH), 7.82 (1 H, s, ArH), 7.70 (1 H, s, |
| | ArH), 7.60 (1 H, s, ArH), 7.50 (1 H, d, ArH), 7.42 (1 H, dd, ArH), 7.19 (1 H, d, ArH), |
| | 5.92 (1 H, d, ArH), 4.24 (2 H, q, CH₂), 3.55 (3 H, s, CH₃), 3.47 (3 H, s, CH₃), 2.55 (2 |
| | H, q, CH₂), 1.55 (3 H, t, CH₃), 1.19 (3 H, t, CH₃). |
| 242 | ¹H NMR δ (ppm)(CHCl₃-d): 8.73 (1 H, d, ArH), 7.83 (1 H, s, ArH), 7.66 (1 H, s, |
| | ArH), 7.61 (1 H, s, ArH), 7.49 (1 H, d, ArH), 7.44-7.39 (1 H, m, ArH), 7.20 (1 H, d, |
| | ArH), 5.93 (1 H, d, ArH), 4.75 (2 H, d, CH), 4.44 (2 H, d, CH), 4.38 (2 H, s, CH), |
| | 3.56 (3 H, s, CH₃), 3.46 (3 H, s, CH₃), 2.56 (2 H, q, CH₂), 1.31 (3 H, s, CH₃), 1.19 (3 |
| | H, t, CH₃). |
| 243 | ¹H NMR δ (ppm)(DMSO-d₆): 8.48 (1 H, d, ArH), 7.98 (1 H, s, ArH), 7.75 (1 H, t, |
| | NH), 7.52-7.23 (1 H, m, ArH), 7.27 (1 H, dd, ArH), 7.15 (1 H, d, ArH), 6.14 (1 H, s, |
| | ArH), 4.12 (2 H, d, CH₂), 3.59 (3 H, s, CH₃), 3.32 (3 H, s, CH₃), 3.04-2.99 (4 H, m, |
| | CH), 2.44 (2 H, q, CH₂), 1.47 (6 H, s, CH), 1.10 (3 H, t, CH₃). |
| 244 | ¹H NMR δ (ppm)(DMSO-d₆): 8.48 (1 H, d, ArH), 8.37 (1 H, s, NH), 7.98 (1 H, s, |
| | ArH), 7.37 (1 H, d, ArH), 7.28 (1 H, dd, ArH), 7.18 (1 H, d, ArH), 6.15 (1 H, s, ArH), |
| | 4.42 (2 H, q, CH₂), 4.26 (2 H, s, CH₂), 3.59 (3 H, s, CH₃), 3.33 (3 H, s, CH₃), 2.45 (2 |
| | H, q, CH₂), 1.10 (3 H, t, CH₃). |
| 245 | ¹H NMR δ (ppm)(DMSO-d₆): 8.49 (1 H, s, ArH), 7.98 (1 H, s, ArH), 7.61 (1 H, t, |
| | NH), 7.37 (1 H, s, ArH), 7.28 (1 H, dd, ArH), 7.17 (1 H, d, ArH), 6.12 (1 H, s, ArH), |
| | 4.21 (2 H, d, CH₂), 3.59 (3 H, s, CH₃), 3.33 (3 H, s, CH₃), 3.18-3.07 (1 H, m, CH), |
| | 2.45 (2 H, m, CH₂), 1.25 (6 H, d, CH₃), 1.10 (3 H, t, CH₃). |
| 246 | ¹H NMR δ (ppm)(CHCl₃-d): 8.73 (1 H, d, ArH), 7.63 (1 H, s, ArH), 7.40 (1 H, d, |
| | ArH), 7.34-7.30 (1 H, m, ArH), 7.18 (1 H, d, ArH), 6.15-6.12 (1 H, m, CH), 5.91 (1 |
| | H, d, ArH), 4.09 (2 H, d, CH), 3.66-3.62 (2 H, m, CH), 3.57 (3 H, s, CH₃), 3.46 (3 H, |
| | s, CH₃), 3.31-3.22 (1 H, m, CH), 2.68 (2 H, d, CH), 2.53 (2 H, q, CH₂), 1.45-1.36 (6 |
| | H, m, CH₃), 1.28-1.14 (3 H, m, CH₃). |
| 247 | ¹H NMR δ (ppm)(CHCl₃-d): 8.72 (1 H, d, ArH), 7.62 (1 H, s, ArH), 7.39 (1 H, d, |
| | ArH), 7.32 (1 H, dd, ArH), 7.19 (1 H, d, ArH), 6.16-6.10 (1 H, m, CH), 5.92 (1 H, s, |
| | ArH), 4.23 (2 H, d, CH), 4.02 (2 H, s, CH), 3.83-3.77 (2 H, m, CH), 3.57 (3 H, s, |
| | CH₃), 3.44 (3 H, s, CH₃), 2.78-2.76 (2 H, m, CH), 2.54 (2 H, q, CH₂), 1.17 (3 H, t, |
| | CH₃). |
| 248 | ¹H NMR δ (ppm)(CHCl₃-d): 8.73 (1 H, s, ArH), 7.63 (1 H, s, ArH), 7.39 (1 H, s, |
| | ArH), 7.31 (1 H, s, ArH), 7.19 (1 H, d, ArH), 6.17 and 6.06 (1 H, 2 s, CH), 5.92 (1 H, |
| | s, ArH), 4.60-3.63 (6 H, m, CH, OH), 3.57 (3 H, s, CH₃), 3.45 (3 H, s, CH₃), 2.67 (2 |
| | H, s, CH), 2.54 (2 H, q, CH₂), 1.40 and 1.37 (3 H, 2 d, CH₃), 1.17 (3 H, t, CH₃), |
| | (rotamers observed). |
| 249 | ¹H NMR δ (ppm)(CHCl₃-d): 8.73 (1 H, s, ArH), 7.62 (1 H, s, ArH), 7.39 (1 H, s, |
| | ArH), 7.35-7.29 (1 H, m, ArH), 7.18 (1 H, d, ArH), 6.16 and 6.09 (1 H, 2 s, CH), |
| | 5.91 (1 H, s, ArH), 5.58 (1 H, d, CH), 4.78-4.62 (1 H, m, NH), 4.38-4.13 (2 H, m, |
| | CH), 3.90-3.71 (2 H, m, CH), 3.56 (3 H, s, CH₃), 3.45 (3 H, s, CH₃), 2.78-2.60 (2 H, |
| | m, CH), 2.54 (2 H, q, CH₂), 1.45 (9 H, s, CH₃), 1.37 and 1.34 (3 H, 2 d, CH₃), 1.17 (3 |
| | H, t, CH₃), (rotamers observed). |
| 250 | ¹H NMR δ (ppm)(CHCl₃-d): 8.72 (1 H, d, ArH), 7.64 (1 H, s, ArH), 7.40 (1 H, s, |
| | ArH), 7.33 (1 H, d, ArH), 7.18 (1 H, d, ArH), 6.18 and 6.13 (1 H, 2 s, CH), 5.93 (1 |
| | H, s, ArH), 4.36 (1 H, s, CH), 4.32-4.18 (2 H, m, CH), 4.00-3.79 (2 H, m, CH), |
| | 3.57 (3 H, s, CH₃), 3.45 (3 H, s, CH₃), 3.38 (3 H, d, CH₃), 2.65 (2 H, s, CH), 2.54 (2 H, q, |
| | CH₂), 1.47 and 1.43 (3 H, 2 d, CH₃), 1.17 (3 H, t, CH₃), (rotamers observed). |
| 251 | ¹H NMR δ (ppm)(CHCl₃-d): 8.72 (1 H, s, ArH), 7.63 (1 H, s, ArH), 7.39 (1 H, s, |
| | ArH), 7.32 (1 H, d, ArH), 7.19 (1 H, d, ArH), 6.18 and 6.09 (1 H, 2 s, CH), 5.93 (1 H, |
| | s, ArH), 4.60-4.30 (2 H, m, CH), 4.21-3.63 (4 H, m, CH, OH), 3.57 (3 H, s, CH₃), |
| | 3.45 (3 H, s, CH₃), 2.67 (2 H, s, CH), 2.54 (2 H, q, CH₂), 1.41 and 1.37 (3 H, 2 d, |
| | CH₃), 1.17 (3 H, t, CH₃), (rotamers observed). |
| 252 | ¹H NMR δ (ppm)(CHCl₃-d): 8.73 (1 H, d, ArH), 7.63 (1 H, s, ArH), 7.32-7.13 (3 H, |
| | m, ArH), 5.90 (1 H, s, ArH), 4.99 (1 H, s, NH), 4.39 (2 H, d, CH₂), 3.58 (3 H, s, CH₃), |
| | 3.45 (3 H, s, CH₃), 2.53 (2 H, q, CH₂), 1.51 (9 H, s, CH₃), 1.16 (3 H, t, CH₃). |
| 253 | ¹H NMR δ (ppm)(DMSO-d₆): 8.49 (1 H, s, ArH), 7.98 (1 H, s, ArH), 7.64 (1 H, t, |
| | NH), 7.37 (1 H, s, ArH), 7.28 (1 H, dd, ArH), 7.17 (1 H, d, ArH), 6.11 (1 H, s, ArH), |
| | 4.18 (2 H, d, CH₂), 3.58 (3 H, s, CH₃), 3.37 (3 H, s, CH₃), 2.99 (2 H, q, CH₂), 2.45 (2 |
| | H, m, CH₂), 1.20 (3 H, t, CH₃), 1.10 (3 H, t, CH₃). |
| 254 | ¹H NMR δ (ppm)(DMSO-d₆): 8.54 (1 H, s, ArH), 8.48-8.36 (2 H, m, NH, ArH), |
| | 7.35 (1 H, s, ArH), 7.28-7.19 (2 H, m, ArH), 6.67 (1 H, s, ArH), 4.37 (2 H, d, CH₂), |
| | 3.90 (2 H, s, CH₂), 3.73 (3 H, s, CH₃), 3.37 (3 H, s, CH₃), 3.36 (3 H, s, CH₃), 2.45 (2 H, q, |
| | CH₂), 1.10 (3 H, t, CH₃). |
| 255 | ¹H NMR δ (ppm)(DMSO-d₆): 8.81 (1 H, t, NH), 8.55 (1 H, s, ArH), 8.43 (1 H, s, |
| | ArH), 7.37 (1 H, s, ArH), 7.30-7.21 (2 H, m, ArH), 6.71 (1 H, s, ArH), 4.37 (2 H, d, |
| | CH₂), 3.75 (5 H, m, CH₂, CH₃), 3.38 (3 H, s, CH₃), 2.46 (2 H, q, CH₂), 1.12 (3 H, t, |
| | CH₃). |
| 256 | ¹H NMR δ (ppm)(DMSO-d₆): 9.39 (1 H, t, NH), 8.51 (1 H, s, ArH), 8.19-8.10 (1 H, |
| | m, ArH), 7.33 (1 H, s, ArH), 7.26-7.17 (2 H, m, ArH), 6.39 (1 H, s, ArH), 6.32 (1 H, |
| | t, CH), 4.41 (2 H, d, CH₂), 3.66 (3 H, s, CH₃), 3.34 (3 H, s, CH₃), 2.44 (2 H, q, CH₂), |
| | 1.10 (3 H, t, CH₃). |
| 257 | ¹H NMR δ (ppm)(CHCl₃-d): 9.5 (1 H, s, NH), 8.72 (1 H, d, ArH), 8.25 (1 H, s, ArH), |
| | 7.64 (1 H, s, ArH), 7.40 (1 H, d, ArH), 7.32 (1 H, dd, ArH), 7.20 (1 H, d, ArH), |
| | 6.14 (1 H, s, CH), 5.94 (1 H, s, ArH), 4.28 (2 H, s, CH), 3.89-3.83 (2 H, m, CH), 3.58 (3 |
| | H, s, CH₃), 3.45 (3 H, s, CH₃), 2.73 (2 H, s, CH), 2.55 (2 H, q, CH₂), 1.17 (3 H, t, |
| | CH₃). |
| 258 | ¹H NMR δ (ppm)(DMSO-d₆): 10.3 (1 H, s, NH), 8.48 (1 H, s, ArH), 7.98 (1 H, s, |
| | ArH), 7.64 (1 H, s, NH), 7.31-7.24 (1 H, m, ArH), 7.23-7.11 (2 H, m, ArH), |
| | 6.19-6.13 (1 H, m, ArH), 4.28 (2 H, d, CH₂), 3.60 (3 H, s, CH₃), 3.33 (3 H, s, CH₃), 2.44 (2 H, |
| | m, q, CH₂), 1.09 (3 H, t, CH₃). |
| 264 | ¹H NMR δ (ppm)(CHCl₃-d): 8.75 (1 H, s, ArH), 8.43 (1 H, s, ArH), 7.67 (1 H, s, |
| | ArH), 7.62 (1 H, s, ArH), 7.45 (1 H, d, ArH), 7.32 (1 H, s, ArH), 7.18-7.14 (1 H, m, |
| | ArH), 6.22 (1 H, s, ArH), 3.93 (3 H, s, CH₃), 3.64 (3 H, s, CH₃), 3.48 (3 H, s, CH₃). |
| 269 | ¹H NMR δ (ppm)(DMSO-d₆): 8.44 (1 H, s, ArH), 8.01 (1 H, s, ArH), 7.60 (1 H, s, |
| | ArH), 7.35 (1 H, t, NH), 7.24-7.16 (2 H, m, ArH), 6.55 (1 H, s, ArH), 4.17 (2 H, d, |
| | CH₂), 3.65 (3 H, s, CH₃), 3.34 (3 H, s, CH₃), 2.90 (3 H, s, CH₃). |
| 274 | ¹H NMR δ (ppm)(CHCl₃-d): 8.71 (1 H, s, ArH), 7.79 (1 H, s, ArH), 7.70 (1 H, s, |
| | ArH), 7.66 (1 H, s, ArH), 7.45-7.33 (3 H, m, ArH), 6.44 (1 H, t, CH), 6.33 (1 H, s, |
| | ArH), 3.99 (3 H, s, CH₃), 3.70 (3 H, s, CH₃), 3.46 (3 H, s, CH₃). |
| 276 | ¹H NMR δ (ppm)(DMSO-d₆): 8.43 (1 H, s, ArH), 8.24 (1 H, s, ArH), 8.01 (1 H, s, |
| | ArH), 7.10 (1 H, s, ArH), 6.43 (1 H, s, ArH), 3.79 (3 H, s, CH₃), 3.76-3.64 (5 H, m, |
| | CH₂, CH₃), 3.30 (3 H, s, CH₃), 2.94-2.79 (6 H, m, CH, CH₂, CH₃), 2.03 (2 H, d, CH₂), |
| | 1.87-1.80 (2 H, m, CH₂). |
| 277 | ¹H NMR δ (ppm)(DMSO-d₆): 8.43 (1 H, d, ArH), 8.01 (1 H, s, ArH), 7.38-7.26 (2 H, |
| | m, ArH), 7.22 (1 H, dd, ArH), 6.59 (1 H, s, ArH), 4.21-4.13 (2 H, m, CH), |
| | 3.99-3.84 (3 H, m, CH), 3.66 (3 H, s, CH₃), 3.34 (3 H, s, CH₃), 3.06 (3 H, s, CH₃). |
| 280 | ¹H NMR δ (ppm)(DMSO-d₆): 8.47 (1 H, s, ArH), 8.29 (2 H, d, ArH), 8.01 (2 H, d, |
| | ArH), 7.65 (1 H, s, ArH), 6.37 (1 H, s, ArH), 3.90 (3 H, s, CH₃), 3.65 (3 H, s, CH₃), |
| | 3.38 (3 H, s, CH₃), 2.56-2.44 (2 H, q, CH₂), 1.14 (3 H, t, CH₃). |
| 283 | ¹H NMR δ (ppm)(DMSO-d₆): 8.43 (1 H, d, ArH), 8.00 (1 H, s, ArH), 7.75-7.69 (1 H, |
| | m, ArH), 7.31 (1 H, t, NH), 7.13-7.07 (2 H, m, ArH), 6.55 (1 H, s, ArH), 4.18 (2 H, d, |
| | CH₂), 3.65 (3 H, s, CH₃), 3.54 (3 H, s, CH₃), 2.47 (3 H, s, CH₃). |
| 284 | ¹H NMR δ (ppm)(DMSO-d₆): 8.63 (1 H, s, ArH), 8.26 (1 H, s, ArH), 7.48 (1 H, d, |
| | ArH), 7.43-7.34 (2 H, m, ArH), 7.29 (1 H, d, ArH), 6.40 (1 H, t, CH), 6.01 (1 H, s, |
| | ArH), 4.25 (2 H, s, CH₂), 3.57 (3 H, s, CH₃), 2.44 (2 H, q, CH₂), 1.09 (3 H, t, CH₃). |
| 287 | ¹H NMR δ (ppm)(DMSO-d₆): 8.41 (1 H, d, ArH), 7.99 (1 H, s, ArH), 7.27 (1 H, d, |
| | ArH), 7.21-7.14 (2 H, m, ArH), 6.99 (1 H, t, CH), 6.50 (1 H, d, ArH), 3.71-3.62 (5 H, |
| | m, CH, CH₃), 2.88 (3 H, s, CH₃), 2.74-2.63 (1 H, m, CH), 2.84-2.76 (2 H, m, CH), |
| | 2.47 (3 H, s, CH₃), 1.91 (2 H, d, CH), 1.74-1.66 (2 H, m, CH). |
| 288 | ¹H NMR δ (ppm)(CHCl₃-d): 8.76 (1 H, s, ArH), 8.48 (1 H, s, ArH), 7.96 (1 H, d, |
| | ArH), 7.72 (1 H, d, ArH), 7.67 (2 H, s, ArH), 7.45 (1 H, dd, ArH), 6.01 (1 H, s, ArH), |
| | 3.61 (3 H, s, CH₃), 3.52 (3 H, s, CH₃), 2.60 (2 H, q, CH₂), 1.24 (3 H, t, CH₃). |
| 289 | ¹H NMR δ (ppm)(DMSO-d₆): 8.48 (1 H, s, ArH), 8.31 (1 H, s, ArH), 8.02 (1 H, s, |
| | ArH), 7.92 (1 H, s, ArH), 7.84 (1 H, d, ArH), 7.64 (1 H, d, ArH), 7.21-7.17 (1 H, m, |
| | ArH), 6.44 (1 H, s, ArH), 3.67 (3 H, s, CH₃), 3.39 (3 H, s, CH₃), 2.54-2.42 (2 H, m, |
| | CH₂), 1.15 (3 H, t, CH₃). |
| 290 | ¹H NMR δ (ppm)(DMSO-d₆): 8.44 (1 H, d, ArH), 7.94 (1 H, s, ArH), 7.33-7.26 (2 H, |
| | m, ArH), 7.15 (1 H, d, ArH), 6.14 (1 H, s, ArH), 4.22-4.15 (2 H, m, CH), 4.00-3.84 (3 |
| | H, m, CH), 3.56 (3 H, s, CH₃), 3.35 (3 H, s, CH₃), 3.06 (3 H, s, CH₃), 2.49-2.38 (2 H, |
| | m, CH₂), 1.06 (3 H, t, CH₃). |
| 299 | ¹H NMR δ (ppm) (CDCl₃): 8.73 (1 H, s, ArH), 7.62 (1 H, s, ArH), 7.16 (1 H, d, ArH), |
| | 6.96 (1 H, d, ArH), 6.86 (1 H, dd, ArH), 5.88 (1 H, s, ArH), 4.56 (2 H, s, CH₂), |
| | 3.57 (3 H, s, CH₃), 3.42 (3 H, s, CH₃), 2.52 (2H, q, CH₂), 1.16 (3H, t, CH₃). |
| 301 | ¹H NMR δ (ppm) (CDCl₃): 8.72 (1 H, d, ArH), 8.42 (1 H, s, ArH), 7.69 (1 H, s, ArH), |
| | 7.68 (1 H, s, ArH), 7.63 (1 H, d, ArH), 7.54 (1 H, d, ArH), 6.08 (1 H, d, ArH), 3.64 (3 |
| | H, s, CH₃), 3.48 (3 H, s, CH₃), 2.58 (2H, q, CH₂), 1.22 (3H, t, CH₃). |
| 302 | ¹H NMR δ (ppm) (CDCl₃): 8.73 (1 H, d, ArH), 8.38 (1 H, s, ArH), 7.65 (1 H, s, ArH), |
| | 7.62 (1 H, s, ArH), 7.45 (1 H, d, ArH), 6.98 (1 H, d, ArH), 5.99 (1 H, s, ArH), 4.94 (1 |
| | H, s, NH), 4.52 (2 H, d, CH₂), 3.59 (3 H, s, CH₃), 3.48 (3 H, s, CH₃), 2.55 (2H, q, |
| | CH₂), 1.47 (9H, s, (CH₃)3), 1.21 (3H, t, CH₃). |
| 303 | ¹H NMR δ (ppm) (CDCl₃): 8.50 (1 H, ArH), 8.22 (1 H, ArH), 7.87 (1 H, ArH), |
| | 7.61 (1 H, ArH), 7.45 (1 H, ArH), 7.12 (1 H, ArH), 6.17 (1 H, ArH), 4.19 (2 H, CH₂), |
| | 3.62 (3 H, CH₃), 3.40 (3 H, CH₃), 2.48 (2H, CH₂), 1.14 (3H, CH₃). |
| 304 | ¹H NMR δ (ppm) (CDCl₃): 8.72 (1 H, d, ArH), 8.37 (1 H, s, ArH), 7.66 (1 H, s, ArH), |
| | 7.58 (1 H, s, ArH), 7.37 (1 H, d, ArH), 6.95 (1 H, d, ArH), 6.00 (1 H, d, ArH), 3.61 (3 |
| | H, s, CH₃), 3.48 (3 H, s, CH₃), 2.55 (2H, q, CH₂), 1.21 (3H, t, CH₃). |
| 305 | ¹H NMR δ (ppm) (CDCl₃): 8.75 (1 H, d, ArH), 7.70 (1 H, s, ArH), 6.11 (1 H, d, ArH), |
| | 5.90 (1 H, s, ArH), 3.87 (2 H, q, CH₂), 3.64 (3 H, s, CH₃), 3.45 (3 H, s, CH₃), 2.32 (3 |
| | H, s, CH₃), 1.35 (3 H, t, CH₃). |
| 306 | ¹H NMR δ (ppm) (CDCl₃): 8.75 (1 H, d, ArH), 7.73 (1 H, s, ArH), 6.10 (1 H, d, ArH), |
| | 5.78 (1 H, s, ArH), 3.66 (3 H, s, CH₃), 3.56 (3 H, s, CH₃), 3.43 (3 H, s, CH₃), |
| | 1.98-1.91 (1 H, m, CH), 0.97, 0.92 (2 H, m, CH₂), 0.78-0.74 (2 H, m, CH₂). |
| 307 | ¹H NMR δ (ppm) (CD₃OD): 8.53 (1 H, s, ArH), 8.06 (1 H, s, ArH), 7.80 (2 H, d, |
| | ArH), 7.41 (2 H, d, ArH), 6.10 (1 H, d, ArH), 5.58 (1 H, s, ArH), 5.57 (1 H, s, ArH), |
| | 3.98 (2 H, q, CH₂), 3.76 (3 H, s, CH₃), 3.47 (3 H, s, CH₃), 1.39 (3 H, t, CH₃). |
| 308 | ¹H NMR δ (ppm) (CDCl₃): 8.74 (1 H, s, ArH), 7.71 (1 H, s, ArH), 7.50 (1 H, s, ArH), |
| | 6.36 (1 H, s, ArH), 4.48 (2 H, d, CH₂), 4.19 (2 H, d, CH₂), 3.68 (3 H, s, CH₃), 3.52 (3 |
| | H, s, CH₃), 3.30 (2 H, s, CH₂), 2.27 (2H, q, CH₂), 1.47 (9H, s, (CH₃)3), 1.11 (3 H, t, |
| | CH₃). |
| 310 | ¹H NMR δ (ppm) (CDCl₃): 8.87 (1 H, s, ArH), 8.07 (1 H, s, ArH), 7.55 (1 H, s, ArH), |
| | 6.55 (1 H, s, ArH), 4.59 (2 H, d, CH₂), 4.15 (2 H, d, CH₂), 3.79 (3 H, s, CH₃), 3.55 (3 |
| | H, s, CH₃), 3.41 (2 H, s, CH₂), 3.00 (3 H, s, CH₃), 2.33 (2H, q, CH₂), 1.16 (3 H, t, |
| | CH₃). |
| 311 | ¹H NMR δ (ppm) (CDCl₃): 8.71 (1 H, s, ArH), 8.35 (1 H, s, ArH), 7.62 (1 H, s, ArH), |
| | 7.58 (1 H, s, ArH), 7.40 (1 H, d, ArH), 6.77 (1 H, s, ArH), 5.96 (1 H, s, ArH), 3.56 (3 |
| | H, s, CH₃), 3.46 (3 H, s, CH₃), 2.53 (3 H, s, CH₃), 2.52 (2 H, q, CH₂), 1.19 (3 H, t, |
| | CH₃). |
| 312 | ¹H NMR δ (ppm) (CDCl₃): 8.72 (1 H, s, ArH), 8.47 (1 H, s, ArH), 7.66 (1H, s, ArH), |
| | 6.95 (1 H, s, ArH), 6.24 (1H, s, ArH), 3.86 (3 H, s, CH₃), 3.80 (3 H, s, CH₃), 3.64 (3 |
| | H, s, CH₃), 3.46 (3 H, s, CH₃), 2.50 (3 H, s, CH₃), 2.44 (3 H, s, CH₃). |
| 313 | ¹H NMR δ (ppm) (DMSO-d₆): 8.44 (1 H, s, ArH), 7.92 (1 H, s, ArH), 7.06 (1H, d, |
| | ArH), 6.67 (1 H, s, ArH), 6.55 (1H, dd, ArH), 6.08 (1H, s, ArH), 3.76-3.73 (4 H, m, |
| | 2xCH₂), 3.68 (3 H, s, CH₃), 3.55 (3 H, s, CH₃), 3.25 (3 H, s, CH₃), 3.18-3.15 (4 H, s, |
| | 2xCH₂). |
| 315 | ¹H NMR δ (ppm) (DMSO-d₆): 8.44 (1 H, s, ArH), 8.03 (1 H, s, ArH), 7.63 (1H, dd, |
| | ArH), 7.50 (1 H, d, ArH), 7.38 (1H, d, ArH), 6.62 (1 H, s, ArH), 3.83 (3 H, s, CH₃), |
| | 3.67 (3 H, s, CH₃), 3.32 (3 H, s, CH₃), 3.65-3.62 (4 H, m, 2xCH₂), 2.88-2.86 (4 H, m, |
| | 2xCH₂). |
| 316 | ¹H NMR δ (ppm) (CDCl₃): 8.73 (1 H, s, ArH), 7.66 (1H, dd, ArH), 7.36 (1 H, s, |
| | ArH), 6.27 (1 H, s, ArH), 4.33 (2 H, t, CH₂), 3.64 (3 H, s, CH₃), 3.50 (3 H, s, CH₃), |
| | 3.00 (2 H, t, CH₂), 2.24 (2 H, q, CH₂), 1.09 (3 H, t, CH₃). |
| 317 | ¹H NMR δ (ppm) (DMSO-d₆): 8.44 (1 H, s, ArH), 8.32 (1 H, s, ArH), 8.02 (1 H, s, |
| | ArH), 7.38 (1 H, s, ArH), 6.44 (1 H, s, ArH), 4.38 (2H, s, CH₂), 3.67 (3 H, s, CH₃), |
| | 3.35 (3 H, s, CH₃), 3.00 (3 H, s, CH₃), 2.82 (3H, s, CH₃), 2.44 (2 H, q, CH₂), 1.08 (3 |
| | H, t, CH₃). |
| 318 | ¹H NMR δ (ppm) (DMSO-d₆): 8.46 (1 H, s, ArH), 8.34 (1 H, s, ArH), 8.18 (1 H, s, |
| | ArH), 8.00 (1 H, s, ArH), 6.33 (1 H, s, ArH), 4.02 (2H, t, CH₂), 3.65 (3 H, s, CH₃), |
| | 3.36 (3 H, s, CH₃), 2.60 (2 H, t, CH₂), 2.43 (2 H, q, CH₂), 2.06 (2 H, quint, CH₂), |
| | 1.08 (3 H, t, CH₃). |
| 321 | ¹H NMR δ (ppm) (DMSO-d₆): 8.56 (1 H, s, ArH), 8.05 (1 H, s, ArH), 7.68 (1 H, s, |
| | ArH), 7.38 (1 H, s, ArH), 6.50 (1 H, s, ArH), 3.71 (3 H, s, CH₃), 3.51 (3 H, s, CH₃), |
| | 2.60 (2 H, q, CH₂), 1.11 (3 H, t, CH₃). |
| 322 | ¹H NMR δ (ppm) (DMSO-d₆): 8.40 (1 H, s, ArH), 8.05 (1 H, s, ArH), 7.77 (1 H, d, |
| | ArH), 7.71 (1 H, s, ArH), 6.67 (1 H, s, ArH), 3.72 (3 H, s, CH₃), 3.28 (3 H, s, CH₃), |
| | 2.51 (2 H, q, CH₂), 1.08 (3 H, t, CH₃). |
| 323 | ¹H NMR δ (ppm) (DMSO-d₆): 8.42 (1 H, s, ArH), 8.00 (1 H, s, ArH), 7.62 (1 H, t, |
| | NH), 7.17 (1 H, s, ArH), 7.11 (1H, d, ArH), 6.37 (1 H, s, ArH), 4.19 (2H, d, CH₂), |
| | 3.65 (3 H, s, CH₃), 3.27 (3 H, s, CH₃), 2.92 (3H, s, CH₃), 2.49 (2 H, CH₂), 1.08 (3 H, |
| | t, CH₃). |
| 324 | ¹H NMR δ (ppm) (DMSO-d₆): 9.04 (1 H, s, NH), 8.43 (1 H, s, ArH), 8.01 (1 H, s, |
| | ArH), 7.17 (1 H, s, ArH), 7.05 (1H, d, ArH), 7.02 (1 H, t, CH), 6.42 (1 H, s, ArH), |
| | 4.33 (2H, s, CH₂), 3.67 (3 H, s, CH₃), 3.28 (3 H, s, CH₃), 2.48 (2 H, q, CH₂), 1.09 (3 |
| | H, t, CH₃). |
| 325 | ¹H NMR δ (ppm) (DMSO-d₆): 8.46 (1 H, s, ArH), 7.99 (1 H, s, ArH), 7.57 (1 H, t, |
| | NH), 7.42 (1 H, d, ArH), 7.05 (1H, d, ArH), 6.28 (1 H, s, ArH), 4.21 (2H, d, CH₂), |
| | 3.62 (3 H, s, CH₃), 3.31 (3 H, s, CH₃), 2.93 (3H, s, CH₃), 2.39 (2 H, q, CH₂), 1.06 (3 |
| | H, t, CH₃). |
| 326 | ¹H NMR δ (ppm) (DMSO-d₆): 9.34 (1 H, t, NH), 8.46 (1 H, s, ArH), 8.00 (1 H, s, |
| | ArH), 7.30 (1 H, d, ArH), 7.05 (1H, d, ArH), 6.34 (1 H, s, ArH), 6.30 (1 H, t, CH), |
| | 4.41 (2H, d, CH₂), 3.64 (3 H, s, CH₃), 3.32 (3 H, s, CH₃), 2.38 (2 H, q, CH₂), 1.05 (3 |
| | H, t, CH₃). |
| 327 | ¹H NMR δ (ppm) (DMSO-d₆): 8.99 (1 H, s, NH), 8.47 (1 H, s, ArH), 8.00 (1 H, s, |
| | ArH), 7.42 (1 H, d, ArH), 7.05 (1H, d, ArH), 6.94 (1 H, t, CH), 6.31 (1 H, s, ArH), |
| | 4.34 (2H, s, CH₂), 3.64 (3 H, s, CH₃), 3.32 (3 H, s, CH₃), 2.39 (2 H, q, CH₂), 1.07 (3 |
| | H, t, CH₃). |
| 328 | ¹H NMR δ (ppm) (CDCl₃): 8.69 (1 H, d, ArH), 8.43 (1 H, s, ArH), 7.67 (1 H, s, ArH), |
| | 7.19 (1 H, s, ArH), 6.01 (1 H, d, ArH), 4.34-4.27 (4 H, m, 2xCH₂), 4.02-3.94 (1 H, m, |
| | CH), 3.64 (3 H, s, CH₃), 3.44 (3 H, s, CH₃), 3.03 (3 H, s, CH₃), 2.51 (2 H, q, CH₂), |
| | 1.16 (3 H, t, CH₃). |
| 329 | ¹H NMR δ (ppm) (DMSO-d₆): 8.44 (1 H, s, ArH), 8.42 (1 H, s, ArH), 8.02 (1 H, s, |
| | ArH), 7.35 (1H, s, ArH), 7.23 (1 H, t, CH), 6.45 (1 H, s, ArH), 4.44 (4H, d, 2xCH₂), |
| | 4.18 (1 H, quint, CH), 3.68 (3 H, s, CH₃), 3.36 (3 H, s, CH₃), 2.41 (2 H, q, CH₂), |
| | 1.08 (3 H, t, CH₃). |
| 330 | ¹H NMR δ (ppm) (DMSO-d₆): 8.44 (1 H, s, ArH), 8.38 (1 H, s, ArH), 8.02 (1 H, s, |
| | ArH), 7.34 (1 H, s, ArH), 6.43 (1 H, s, ArH), 4.24 (2 H, t, CH₂), 4.13 (2 H, t, CH₂), |
| | 4.07 (1 H, q, CH), 3.67 (3 H, s, CH₃), 3.35 (3 H, s, CH₃), 2.42 (2 H, t, CH₂), 1.27 (6 |
| | H, d, 2xCH₃), 1.08 (3 H, t, CH₃). |
| 331 | ¹H NMR δ (ppm) (CDCl₃): 8.72 (1 H, s, ArH), 8.42 (1 H, s, ArH), 7.65 (1 H, s, ArH), |
| | 7.29 (1 H, s, ArH), 5.97 (1 H, s, ArH), 3.93-3.85 (3 H, m, CH₂ and CH), 3.75-3.68 (2 |
| | H, m, CH₂), 3.61 (3 H, s, CH₃), 3.56 (2 H, s, CH₂), 3.46 (3 H, s, CH₃), 2.52 (2 H, q, |
| | CH₂), 1.18 (3 H, t, CH₃). |
| 332 | ¹H NMR δ (ppm) (CDCl₃): 8.79 (1 H, s, ArH), 8.48 (1 H, s, ArH), 7.96 (1 H, s, ArH), |
| | 7.32 (1 H, s, ArH), 6.14 (1 H, s, ArH), 4.54 (1 H, t, CH), 4.49-4.42 (2 H, m, 2xCH), |
| | 4.25 (1 H, t, CH), 4.09-4.04 (1 H, m, CH), 3.71 (3 H, s, CH₃), 3.56 (3 H, s, CH₃), |
| | 2.54 (2 H, q, CH₂), 1.19 (3 H, t, CH₃). |
| 333 | ¹H NMR δ (ppm) (DMSO-d₆): 8.44 (1 H, s, ArH), 8.39 (1 H, s, ArH), 8.02 (1 H, s, |
| | ArH), 7.36 (1 H, s, ArH), 6.44 (1 H, s, ArH), 6.44 (1 H, t, CH), 4.70 (1 H, t, CH), |
| | 4.53 (1 H, t, CH), 4.38 (1 H, t, CH), 4.24-4.09 (2 H, m, 2xCH), 3.67 (3 H, s, CH₃), |
| | 3.35 (3 H, s, CH₃), 2.41 (2 H, q, CH₂), 1.09 (3 H, t, CH₃). |
| 334 | ¹H NMR δ (ppm) (DMSO-d₆): 8.44 (1 H, s, ArH), 8.41 (1 H, s, ArH), 8.02 (1 H, s, |
| | ArH), 7.38 (1 H, s, ArH), 6.45 (1 H, s, ArH), 4.80 (1 H, t, CH), 4.60 (1 H, t, CH), |
| | 4.46 (1 H, dd, CH), 4.25 (1 H, dd, CH), 4.20-4.13 (1 H, m, CH), 3.68 (3 H, s, CH₃), |
| | 3.36 (3 H, s, CH₃), 2.41 (2 H, q, CH₂), 1.09 (3 H, t, CH₃). |
| 335 | ¹H NMR δ (ppm) (DMSO-d₆): 8.44 (1 H, s, ArH), 8.37 (1 H, s, ArH), 8.01 (1 H, s, |
| | ArH), 7.35 (1 H, s, ArH), 6.43 (1 H, s, ArH), 4.51 (1 H, t, CH), 4.37 (1 H, dd, CH), |
| | 4.28 (1 H, t, CH), 4.11 (1 H, dd, CH), 4.08-4.00 (1 H, m, CH), 3.69 (2 H, s, CH₃), |
| | 3.67 (3 H, s, CH₃), 3.35 (3 H, s, CH₃), 2.42 (2 H, q, CH₂), 1.09 (3 H, t, CH₃). |
| 336 | ¹H NMR δ (ppm) (DMSO-d₆): 8.44 (1 H, s, ArH), 8.37 (1 H, s, ArH), 8.01 (1 H, s, |
| | ArH), 7.34 (1 H, s, ArH), 6.42 (1 H, s, ArH), 4.55 (1 H, t, CH), 4.39 (1 H, dd, CH), |
| | 4.24-4.19 (1 H, m, CH), 4.05-3.99 (2 H, m, CH), 3.87-3.83 (2 H, m, CH), 3.67 (3 H, |
| | s, CH₃), 3.35 (3 H, s, CH₃), 2.42 (2 H, q, CH₂), 1.65-1.53 (6 H, m, CH), 1.09 (3 H, t, |
| | CH₃). |
| 339 | ¹H NMR δ (ppm) (DMSO-d₆): 8.44 (1 H, s, ArH), 8.37 (1 H, s, ArH), 8.01 (1 H, s, |
| | ArH), 7.33 (1 H, s, ArH), 6.42 (1 H, s, ArH), 4.29-4.25 (2 H, m, CH), 4.12-4.08 (2 H, |
| | m, CH), 4.04-3.96 (1 H, m, CH), 3.67 (3 H, s, CH₃), 3.59 (3 H, s, CH₃), 3.35 (3 H, s, |
| | CH₃), 2.41 (2 H, q, CH₂), 1.08 (3 H, t, CH₃). |
| 340 | ¹H NMR δ (ppm) (DMSO-d₆): 8.44 (1 H, s, ArH), 8.37 (1 H, s, ArH), 8.01 (1 H, s, |
| | ArH), 7.34 (1 H, s, ArH), 6.43 (1 H, s, ArH), 4.49 (1 H, t, CH), 4.32 (1 H, dd, CH), |
| | 4.25 (1 H, t, CH), 4.08 (1 H, dd, CH), 4.06-4.00 (1 H, m, CH), 3.67 (3 H, s, CH₃), |
| | 3.35 (3 H, s, CH₃), 3.27 (3 H, s, CH₃), 3.05-2.96 (1 H, m, CH), 2.78-2.66 (4 H, m, |
| | CH), 2.41 (2 H, q, CH₂), 1.09 (3 H, t, CH₃). |
| 343 | ¹H NMR δ (ppm) (DMSO-d₆): 8.43 (1 H, s, ArH), 8.30 (1 H, s, ArH), 8.01 (1 H, s, |
| | ArH), 7.41 (1 H, s, ArH), 7.04 (1H, t, CH), 6.43 (1 H, s, ArH), 4.39 (2H, s, CH₂), |
| | 3.67 (3 H, s, CH₃), 3.35 (3 H, s, CH₃), 2.93 (3H, s, CH₃), 2.43 (2 H, CH₂), 1.08 (3 H, t, |
| | CH₃). |
| 345 | ¹H NMR δ (ppm) (DMSO-d₆): 8.69 (1 H, s, ArH), 8.22 (1 H, s, ArH), 7.56-7.52 (3 H, |
| | m, 2ArH and CH), 7.28 (1 H, s, ArH), 7.11 (2 H, d, ArH), 6.22 (1H, d, CH), 3.76 (3 |
| | H, s, CH₃), 3.47 (3 H, s, CH₃). |
| 346 | ¹H NMR δ (ppm) (CDCl₃): 8.70 (1 H, s, ArH), 7.62 (1 H, s, ArH), 7.28 (1 H, d, ArH), |
| | 7.22 (2 H, t, ArH), 5.96 (1 H, t, CH), 5.91 (1H, s, ArH), 4.84 (1H, t, CH), 4.57 (1H, t, |
| | CH), 4.51 (1H, dd, CH), 4.25 (1H, dd, CH), 4.04-3.96 (1 H, m, CH), 3.58 (3 H, s, |
| | CH₃), 3.43 (3 H, s, CH₃), 2.54 (2 H, q, CH₂), 1.16 (3 H, t, CH₃). |
| 347 | ¹H NMR δ (ppm) (CDCl₃): 8.70 (1 H, s, ArH), 7.62 (1 H, s, ArH), 7.28 (1 H, d, ArH), |
| | 7.23 (2 H, t, ArH), 5.92 (1 H, s, ArH), 4.85 (1H, t, CH), 4.59 (1H, t, CH), 4.50 (1H, |
| | dd, CH), 4.29 (1H, dd, CH), 4.06-3.98 (1 H, m, CH), 3.58 (3 H, s, CH₃), 3.43 (3 H, s, |
| | CH₃), 2.54 (2 H, q, CH₂), 1.16 (3 H, t, CH₃). |
| 349 | ¹H NMR δ (ppm) (CDCl₃): 8.70 (1 H, s, ArH), 7.65 (1 H, s, ArH), 7.31-7.26 (2 H, m, |
| | ArH), 7.18 (1 H, d, ArH), 5.89 (1 H, s, ArH), 4.30 (2H, t, CH), 4.16 (2H, t, CH), |
| | 4.02-3.96 (1 H, m, CH), 3.81 (2H, t, CH₂), 3.57 (3 H, s, CH₃), 3.42 (3 H, s, CH₃), |
| | 3.40 (3H, s, CH₃), 3.31 (2H, t, CH₂), 2.51 (2 H, q, CH₂), 1.14 (3 H, t, CH₃). |
| 350 | ¹H NMR δ (ppm) (CDCl₃): 8.72 (1 H, s, ArH), 7.61 (1 H, s, ArH), 7.29 (1 H, d, ArH), |
| | 7.21 (2 H, t, ArH), 5.91 (1 H, d, ArH), 4.70 (1H, t, CH), 4.51 (1H, t, CH), 4.35 (1H, |
| | dd, CH), 4.20 (1H, dd, CH), 4.05 (2 H, s, CH₂), 3.96-3.88 (1 H, m, CH), 3.58 (3 H, s, |
| | CH₃), 3.44 (6 H, s, CH₃), 2.54 (2 H, q, CH₂), 1.16 (3 H, t, CH₃). |
| 351 | ¹H NMR δ (ppm) (CDCl₃): 8.72 (1 H, s, ArH), 7.61 (1 H, s, ArH), 7.35 (1 H, s, ArH), |
| | 7.31 (1 H, dd, ArH), 7.17 (1 H, d, ArH), 5.90 (1 H, s, ArH), 3.88 (2 H, t, CH), |
| | 3.81-3.74 (1 H, m, CH), 3.57 (2 H, t, CH), 3.57 (3 H, s, CH₃), 3.55 (2 H, s, CH₂), 3.44 (3 |
| | H, s, CH₃), 2.53 (2 H, q, CH₂), 1.17 (3 H, t, CH₃). |
| 352 | ¹H NMR δ (ppm) (CDCl₃): 8.69 (1 H, d, ArH), 7.61 (1 H, s, ArH), 7.29 (1 H, d, ArH), |
| | 7.26 (1 H, dd, ArH), 7.20 (1 H, d, arH), 5.91 (1 H, s, ArH), 4.42 (2 H, dd, CH), |
| | 4.25 (2 H, dd, CH), 3.92-3.85 (1 H, m, CH), 3.82 (2 H, q, CH₂), 3.57 (3 H, s, CH₃), 3.42 (3 |
| | H, s, CH₃), 2.53 (2 H, q, CH₂), 1.15 (3 H, t, CH₃). |
| 353 | ¹H NMR δ (ppm) (CDCl₃): 8.72 (1 H, s, ArH), 7.64 (1 H, s, ArH), 7.32-7.27 (2 H, m, |
| | ArH), 7.22 (1 H, d, ArH), 5.92 (1 H, s, ArH), 4.54 (2 H, t, CH), 4.38 (2 H, t, CH), |
| | 4.04 (3 H, s, CH₃), 4.01-3.93 (1 H, m, CH), 3.59 (3 H, s, CH₃), 3.45 (3 H, s, CH₃), |
| | 2.54 (2 H, q, CH₂), 1.17 (3 H, t, CH₃). |
| 354 | ¹H NMR δ (ppm) (CDCl₃): 8.71 (1 H, s, ArH), 7.62 (1 H, s, ArH), 7.32-7.27 (2 H, m, |
| | ArH), 7.22 (1 H, d, ArH), 6.23 (1 H, t, CH), 5.92 (1 H, s, ArH), 4.57 (2 H, t, CH), |
| | 4.39 (2 H, t, CH), 4.02-3.98 (1 H, m, CH), 3.58 (3 H, s, CH₃), 3.43 (3 H, s, CH₃), |
| | 2.54 (2 H, q, CH₂), 1.17 (3 H, t, CH₃). |
| 355 | ¹H NMR δ (ppm) (DMSO-d₆): 8.49 (1 H, s, ArH), 8.04 (1 H, s, ArH), 7.44 (1 H, t, |
| | ArH), 7.31-7.26 (2 H, m, ArH), 6.58 (1 H, s, ArH), 4.46 (2 H, s, CH₂), 4.40 (2 H, s, |
| | CH₂), 3.66 (3 H, s, CH₃), 3.39 (3 H, s, CH₃), 3.17 (3 H, s, CH₃). |
| 356 | ¹H NMR δ (ppm) (CDCl₃): 8.70 (1 H, s, ArH), 7.63 (1 H, s, ArH), 7.13 (1 H, s, ArH), |
| | 7.06 (1 H, dd, ArH), 5.99 (1 H, s, ArH), 4.33 (2 H, s, CH₂), 3.62 (3 H, s, CH₃), 3.41 (3 |
| | H, s, CH₃), 2.90 (3 H, s, CH₃), 2.87 (3 H, s, CH₃), 2.60-2.53 (2 H, m, CH₂), 1.16 (3 H, |
| | t, CH₃). |
| 357 | ¹H NMR δ (ppm) (CDCl₃): 8.68 (1 H, s, ArH), 7.65 (1 H, s, ArH), 7.16 (1 H, s, ArH), |
| | 7.09 (1 H, dd, ArH), 6.07 (1 H, s, ArH), 4.49 (2 H, s, CH₂), 4.18 (2 H, s, CH₂), 3.64 (3 |
| | H, s, CH₃), 3.40 (3 H, s, CH₃), 3.11 (3 H, s, CH₃), 2.60-2.56 (2 H, m, CH₂), 1.17 (3 H, |
| | t, CH₃). |
| 358 | ¹H NMR δ (ppm) (DMSO-d₆): 8.47 (1 H, s, ArH), 8.04 (1 H, s, ArH), 7.41 (1 H, t, |
| | ArH), 7.22 (1 H, d, ArH), 6.62 (1 H, s, ArH), 4.27 (2 H, s, CH₂), 3.69 (3 H, s, CH₃), |
| | 3.38 (3 H, s, CH₃), 2.99 (3 H, s, CH₃), 2.74 (3 H, s, CH₃). |
| 359 | ¹H NMR δ (ppm) (DMSO-d₆): 8.47 (1 H, s, ArH), 8.04 (1 H, s, ArH), 7.39 (1 H, t, |
| | ArH), 7.24-7.21 (2 H, m, ArH), 6.61 (1 H, s, ArH), 4.58 (2 H, s, CH₂), 4.47 (2 H, s, |
| | CH₂), 3.69 (3 H, s, CH₃), 3.38 (3 H, s, CH₃), 3.26 (3 H, s, CH₃), 3.09 (3 H, s, CH₃). |
| 363 | ¹H NMR δ (ppm) (CDCl₃): 8.66 (1 H, d, ArH), 7.68 (1 H, s, ArH), 7.37 (1 H, d, ArH), |
| | 7.29 (1 H, dd, ArH), 7.23 (1 H, d, ArH), 6.39 (1 H, t, CHF2), 6.36 (1 H, d, ArH), |
| | 6.23 (1 H, t, CHF2), 4.58 (2 H, dd, CH₂), 4.34 (2 H, dd, CH₂), 4.00-3.92 (1 H, m, CH), |
| | 3.71 (3 H, s, CH₃), 3.41 (3 H, s, CH₃). |
| 364 | ¹H NMR δ (ppm) (CDCl₃): 8.66 (1 H, s, ArH), 7.70 (1 H, s, ArH), 7.29 (1 H, d, ArH), |
| | 7.23 (1 H, dd, ArH), 7.19 (1 H, s, ArH), 6.39 (1 H, t, CHF2), 6.31 (1 H, d, ArH), |
| | 3.93 (2 H, t, CH₂), 3.87-3.79 (1 H, m, CH), 3.69 (3 H, s, CH₃), 3.42-3.39 (5 H, m, CH₃ and |
| | CH₂), 3.08 (2 H, q, CH₂). |
| 365 | ¹H NMR δ (ppm) (CDCl₃): 8.66 (1 H, d, ArH), 7.69 (1 H, s, ArH), 7.29 (1 H, d, ArH), |
| | 7.23 (dd, 1H, ArH), 7.20 (1H, s, ArH), 6.39 (1 H, t, CHF2), 6.30 (1 H, s, ArH), |
| | 5.81 (1 H, tt, CHF2), 3.88 (2H, t, CH₂), 3.82-3.74 (1 H, m, CH), 3.69 (3 H, s, CH₃), |
| | 3.41 (3H, s, CH₃), 3.37 (2H, t, CH₂), 2.89 (2H, td, CH₂). |
| 366 | ¹H NMR δ (ppm) (CDCl₃): 8.67 (1 H, d, ArH), 7.69 (1 H, s, ArH), 7.32 (2 H, m, |
| | ArH), 7.26 (s, 1H, ArH), 7.21 (1H, s, ArH), 6.39 (1 H, t, CHF2), 6.31 (1 H, s, ArH), |
| | 4.36 (2 H, t, CH₂), 4.00 (6H, t, 3 * CH₂), 3.83-3.78 (1 H, m, CH), 3.69 (3 H, s, CH₃), |
| | 3.41 (3H, s, CH₃), 2.27 (2H, qt, CH₂). |
| 367 | ¹H NMR δ (ppm) (CDCl₃): 8.68 (1 H, d, ArH), 7.69 (1 H, s, ArH), 7.32 (1 H, d, ArH), |
| | 7.28 (d, 1H, ArH), 7.22 (1 H, m, ArH), 6.40 (1 H, t, CHF2), 6.30 (1 H, s, ArH), |
| | 4.38 (2 H, t, CH₂), 4.04 (2H, t, CH₂), 3.83-3.75 (1 H, m, CH), 3.69 (3 H, s, CH₃), 3.31 (4H, |
| | m, 2xCH₂), 1.62-1.50 (6H, m, 3xCH₂). |
| 369 | ¹H NMR δ (ppm) (CDCl₃): 8.67 (1 H, d, ArH), 7.71 (1 H, s, ArH), 7.35-7.30 (2 H, m, |
| | ArH), 7.27 (1 H, s, ArH), 6.40 (1 H, t, CHF2), 6.33 (1 H, d, ArH), 4.25 (2 H, t, CH₂), |
| | 4.04 (2 H, dd, CH₂), 3.84-3.77 (1 H, m, CH), 3.70 (3 H, s, CH₃), 3.42 (3 H, s, CH₃), |
| | 2.86 (6 H, s, CH₃). |
| 370 | ¹H NMR δ (ppm) (CDCl₃): 8.66 (1 H, d, ArH), 7.72 (1 H, s, ArH), 7.35 (1 H, d, ArH), |
| | 7.30 (1 H, dd, ArH), 7.22 (1 H, s, ArH), 6.39 (1 H, t, CHF2), 6.34 (1 H, d, ArH), |
| | 4.30 (2 H, t, CH₂), 4.10 (2 H, dd, CH₂), 3.88-3.80 (1 H, m, CH), 3.71 (3 H, s, CH₃), 3.41 (3 |
| | H, s, CH₃), 2.94 (3 H, s, CH₃). |
| 372 | ¹H NMR δ (ppm) (CDCl₃): 8.69 (1 H, d, ArH), 7.71 (1 H, s, ArH), 7.33 (2 H, m, |
| | ArH), 7.26 (s, 1H, ArH), 6.41 (1 H, t, CHF2), 6.32 (1 H, s, ArH), 4.23 (2 H, t, CH₂), |
| | 4.02 (2H, t, CH₂), 3.83-3.75 (1 H, m, CH), 3.71 (3 H, s, CH₃), 3.26 (4H, m, 2xCH₂), |
| | 1.68-1.50 (6H, m, 3xCH₂). |
| 373 | ¹H NMR δ (ppm) (CDCl₃): 8.66 (1 H, d, ArH), 7.78 (1 H, s, ArH), 7.34 (1 H, d, ArH), |
| | 7.30 (1 H, dd, ArH), 7.24 (1 H, s, ArH), 6.40 (1 H, t, CHF2), 6.33 (1 H, d, ArH), |
| | 4.31 (2 H, t, CH₂), 4.13 (2 H, dd, CH₂), 3.87-3.80 (1 H, m, CH), 3.71 (3 H, s, CH₃), 3.42 (3 |
| | H, s, CH₃), 3.16 (1 H, qt, CH), 1.40 (3H, s, CH₃), 1.38 (3H, s, CH₃). |
| 374 | ¹H NMR δ (ppm) (CDCl₃): 8.67 (1 H, d, ArH), 7.69 (1 H, s, ArH), 7.30 (1 H, d, ArH), |
| | 7.23 (2 H, m, ArH), 6.94 (1 H, s br, NH), 6.39 (1 H, t, CHF2), 6.33 (1 H, s, ArH), |
| | 5.46 (1H, s br, NH), 3.88 (2 H, t, CH₂), 3.78-3.71 (1 H, m, CH), 3.70 (3 H, s, CH₃), |
| | 3.4 (5 H, t, CH₃ + CH₂), 3.25 (2H, s, CH₂). |
| 375 | ¹H NMR δ (ppm) (CDCl₃): 8.65 (1 H, d, ArH), 7.61 (1 H, s, ArH), 7.34 (1 H, d, ArH), |
| | 7.27 (1 H, dd, ArH), 7.17 (1 H, d, ArH), 5.89 (1 H, d, ArH), 4.83 (1 H, t, NH), 4.26 (2 |
| | H, d, CH₂), 3.55 (3 H, s, CH₃), 3.41 (3 H, s, CH₃), 2.83 (3 H, s, CH₃), 2.51 (2 H, q, |
| | CH₂), 1.13 (3 H, t, CH₃). |
| 376 | ¹H NMR δ (ppm) (CDCl₃): 8.69 (1 H, d, ArH), 7.65 (1 H, s, ArH), 7.37 (1 H, d, ArH), |
| | 7.29 (1 H, dd, ArH), 7.19 (1 H, d, ArH), 5.91 (1 H, d, ArH), 4.66 (1 H, s, NH), |
| | 4.26 (1 H, s, NH), 3.58 (3 H, s, CH₃), 3.42 (3 H, s, CH₃), 2.76 (3 H, s, CH₃), 2.52 (2 H, q, |
| | CH₂), 1.15 (3 H, t, CH₃). |
| 377 | ¹H NMR δ (ppm) (CDCl₃): 8.65 (1 H, s, ArH), 7.58 (1 H, s, ArH), 7.24 (1 H, d, ArH), |
| | 7.17-7.12 (2 H, m, ArH), 6.17 (1 H, t, NH), 5.91 (1 H, d, ArH), 4.47 (2 H, d, CH₂), |
| | 3.56 (3 H, s, CH₃), 3.39 (3 H, s, CH₃), 2.50 (2 H, q, CH₂), 2.28-2.12 (3 H, m, CH), |
| | 1.99-1.77 (6 H, m, CH), 1.12 (3 H, t, CH₃). |
| 378 | ¹H NMR δ (ppm) (CDCl₃): 8.71 (1 H, d, ArH), 7.60 (1 H, s, ArH), 7.29 (1 H, d, ArH), |
| | 7.21 (1 H, dd, ArH), 7.16 (1 H, d, ArH), 5.86 (1 H, d, ArH), 3.78 (2 H, s, CH₂), |
| | 3.66 (4 H, t, CH₂), 3.55 (3 H, s, CH₃), 3.43 (3 H, s, CH₃), 2.51 (2 H, q, CH₂), 1.15 (3 H, t, |
| | CH₃). |
| 379 | ¹H NMR δ (ppm) (CDCl₃): 8.71 (1 H, d, ArH), 7.60 (1 H, s, ArH), 7.33 (1 H, d, ArH), |
| | 7.25 (1 H, dd, ArH), 7.17 (1 H, d, ArH), 5.91 (1 H, tt, CH), 5.86 (1 H, d, ArH), |
| | 3.89 (2 H, s, CH₂), 3.55 (3 H, s, CH₃), 3.43 (3 H, s, CH₃), 3.06 (2 H, td, CH₂), 2.52 (2 H, q, |
| | CH₂), 1.15 (3 H, t, CH₃). |
| 380 | ¹H NMR δ (ppm) (CDCl₃): 8.70 (1 H, d, ArH), 7.60 (1 H, s, ArH), 7.34 (1 H, d, ArH), |
| | 7.26 (1 H, dd, ArH), 7.17 (1 H, d, ArH), 5.86 (1 H, d, ArH), 3.94 (2 H, s, CH₂), |
| | 3.55 (3 H, s, CH₃), 3.43 (3 H, s, CH₃), 3.26 (2 H, q, CH₂), 2.51 (2 H, q, CH₂), 1.15 (3 H, t, |
| | CH₃). |
| 381 | ¹H NMR δ (ppm) (CDCl₃): 8.71 (1 H, d, ArH), 7.60 (1 H, s, ArH), 7.24 (1 H, s, ArH), |
| | 7.18-7.13 (2 H, m, ArH), 5.86 (1 H, d, ArH), 3.65 (2 H, t, CH₂), 3.65 (2 H, s, CH₂), |
| | 3.54 (3 H, s, CH₃), 3.42 (3 H, s, CH₃), 3.40 (2 H, t, CH₂), 3.31 (1 H, quint, CH), |
| | 2.50 (2 H, q, CH₂), 1.14 (3 H, t, CH₃). |
| 382 | ¹H NMR δ (ppm) (CDCl₃): 8.70 (1 H, d, ArH), 7.58 (1 H, s, ArH), 7.26 (1 H, s, ArH), |
| | 7.18 (1 H, dd, ArH), 7.11 (1 H, d, ArH), 5.83 (1 H, d, ArH), 3.65 (2 H, s, CH₂), |
| | 3.52 (3 H, s, CH₃), 3.42 (3 H, s, CH₃), 3.06 (4 H, s, CH₂), 2.49 (2 H, q, CH₂), 1.24 (6 H, s, |
| | CH₃), 1.14 (3 H, t, CH₃). |
| 383 | ¹H NMR δ (ppm) (CDCl₃): 8.70 (1 H, d, ArH), 7.37 (1 H, s, ArH), 7.67 (1 H, s, ArH), |
| | 7.18 (1 H, s, ArH), 5.99 (1 H, d, ArH), 4.02-4.97 (2 H, m, CH₂), 3.62 (3 H, s, CH₃), |
| | 3.45 (3 H, s, CH₃), 2.89-2.82 (2 H, m, CH₂), 2.84 (3 H, s, CH₃), 2.52 (2 H, q, CH₂), |
| | 2.18-2.13 (2 H, m, CH₂), 1.97 (2 H, dq, CH₂), 1.33-1.25 (1 H, m, CH), 1.17 (3 H, t, |
| | CH₃). |
| 384 | ¹H NMR δ (ppm) (DMSO-d₆): 8.43 (d, 1H, ArH), 8.26 (s, 1H, ArH), 8.00 (s, 1H, |
| | ArH), 7.28 (s, 1H, ArH), 6.39 (d, 1H, ArH), 3.76 (s, 2H, CH₂), 3.67 (s, 3H, CH₃), |
| | 2.91 (d, 2H, CH₂), 2.62-2.74 (m, 1H, CH), 2.40 (q, 2H, CH₂), 2.25-2.36 (m, 2H, CH), |
| | 1.88-1.97 (m, 2H, CH), 1.77-1.88 (m, 2H, CH), 1.08 (d, 3H, CH₃) |
| 385 | ¹H NMR δ (ppm) (DMSO-d₆): 8.46 (s, 1H, ArH), 7.99 (s, 1H, ArH), 7.31 (d, 1H, |
| | ArH), 7.23-7.26 (m, 1H, ArH), 7.14-7.20 (m, 1H, ArH), 6.23 (s, 1H, ArH), 3.62 (s, |
| | 3H, CH₃), 3.31 (s, 3H, CH₃), 2.44 (q, 2H, CH₂), 1.72-1.82 (m, 2H, CH), |
| | 1.46-1.60 (m, 2H, CH), 1.08 (t, 3H, CH₃). |
| 386 | ¹H NMR δ (ppm) (DMSO-d₆): 8.47 (s, 1H, ArH), 7.96 (s, 1H, ArH), 7.22 (d, 1H, |
| | ArH), 7.14 (dd, 1H, ArH), 7.09 (d, 1H, ArH), 6.11 (s, 1H, ArH), 3.87 (s, 2H, CH₂), |
| | 3.57 (s, 3H, CH₃), 3.54 (s, 2H, CH₂), 3.43 (s, 2H, CH₂), 3.31 (s, 3H, CH₃), 2.41 (q, |
| | 2H, CH₂), 1.22 (s, 6H, 2xCH₃), 1.06 (t, 3H, CH₃) |
| 387 | ¹H NMR δ (ppm) (DMSO-d₆): 8.48 (s, 1H, ArH), 7.97 (s, 1H, ArH), 7.55-7.59 (m, |
| | 1H, CH), 7.36 (d, 1H, ArH), 7.27 (dd, 1H, ArH), 7.16 (d, 1H, ArH), 6.11 (s, 1H, |
| | ArH), 4.19 (d, 2H, CH₂), 3.58 (s, 3H, CH₃), 3.33 (s, 3H, CH₃), 2.90 (s, 3H, CH₃), |
| | 2.44 (q, 2H, CH₂), 1.09 (t, 3H, CH₃) |
| 389 | ¹H NMR δ (ppm) (DMSO-d₆): 8.46 (s, 1H, ArH), 7.99 (s, 1H, ArH), 7.27 (s, 1H, |
| | ArH), 7.19 (d, 2H, ArH), 6.25 (s, 1H, ArH), 4.90 (d, 2H, CH₂), 4.71 (d, 2H, CH₂), |
| | 3.61 (s, 3H, CH₃), 3.30 (s, 5H, CH₂ + CH₃), 2.44 (q, 2H, CH₂), 1.08 (t, 3H, CH₃) |
| 390 | ¹H NMR δ (ppm) (DMSO-d₆): 8.46 (d, 1H, ArH), 7.99 (s, 1H, ArH), 7.43 (d, 1H, |
| | ArH), 7.37 (dd, 1H, ArH), 7.26 (d, 1H, ArH), 6.24 (s, 1H, ArH), 5.51 (s, 1H, CH), |
| | 3.61 (s, 3H, CH₃), 3.37 (br. s., 3H, CH₃), 3.15-3.24 (m, 4H, 2xCH₂), 2.65 (br. s., |
| | 2H, 2xCH), 2.89 (s, 3H, CH₃), 2.54-2.59 (m, 2H, 2xCH), 2.43-2.48 (m, 2H, CH₂), |
| | 1.08 (t, 3H, CH₃) |
| 391 | ¹H NMR δ (ppm) (DMSO-d₆): 8.45 (d, 1H, ArH), 7.98 (s, 1H, ArH), 7.37 (d, 1H, |
| | ArH), 7.31 (dd, 1H, ArH), 7.23 (d, 1H, ArH), 6.24 (d, 1H, ArH), 5.26 (s, 1H, CH), |
| | 3.61 (s, 3H, CH₃), 3.30-3.62 (m, 4H, 2xCH₂), 2.45 (q, 2H, CH₂), 1.08 (t, 3H, CH₃) |
| 392 | ¹H NMR δ (ppm) (DMSO-d₆): 8.47 (d, 1H, ArH), 8.47 (d, 1H, ArH), 7.96 (s, 1H, |
| | ArH), 7.14-7.18 (m, 2H, 2xArH), 7.06-7.10 (m, 1H, ArH), 6.96 (t, 1H, NH), |
| | 6.12 (s, 1H, ArH), 4.85 (d, 2H, CH₂), 4.67 (d, 2H, CH₂), 3.58 (s, 3H, CH₃), 3.33 (s, 3H, |
| | CH₃), 2.84 (s, 3H, CH₃), 2.76-2.81 (m, 2H, CH₂), 2.43 (q, 2H, CH₂), 2.20-2.27 (m, |
| | 2H, CH₂), 1.08 (t, 3H, CH₃) |
| 393 | ¹H NMR δ (ppm) (CDCl₃): 8.45 (s, 1H, ArH), 7.98 (s, 1H, ArH), 7.42 (d, 1H, ArH), |
| | 7.36 (dd, 1H, ArH), 7.24 (d, 1H, ArH), 6.23 (s, 1H, ArH), 5.39 (s, 1H, CH), |
| | 3.58-3.66 (m, 6H, CH₂), 3.61 (s, 3H, CH₃), 3.32 (s, 3H, CH₃), 2.51-2.59 (m, 2H, CH₂), 2.46 (q, |
| | 2H, CH₂), 1.08 (t, 3H, CH₃) |
| 395 | ¹H NMR δ (ppm) (DMSO-d₆): 8.46 (d, 1H, ArH), 7.98 (s, 1H, ArH), 7.44 (d, 1H, |
| | ArH), 7.38 (dd, 1H, ArH), 7.27 (s, 1H, ArH), 7.24 (s, 1H, ArH), 6.24 (s, 1H, ArH), |
| | 5.53 (s, 1H, CH), 3.61 (s, 3H, CH₃), 3.32 (s, 3H, CH₃), 2.57-2.72 (m, 4H, 2xCH₂), |
| | 2.45 (q, 2H, CH₂), 1.99-2.10 (m, 4H, 2xCH₂), 1.08 (t, 3H, CH₃) |
| 397 | ¹H NMR δ (ppm) (DMSO-d₆): 8.49 (s, 1H, ArH), 8.42 (br. s., 1H, NH), 7.97 (s, 1H, |
| | ArH), 7.15-7.19 (m, 2x1H, ArH), 7.08 (dd, 1H, ArH), 6.92 (t, 1H, CH), 6.08 (s, 1H, |
| | ArH), 4.85 (d, 2H, CH₂), 4.65 (d, 2H, CH₂), 3.56 (s, 3H, CH₃), 3.34 (br. s., 3H, CH₃), |
| | 2.85-3.00 (m, 2H, CH₂), 2.44 (q, 2H, CH₂), 2.21-2.34 (m, 2H, CH₂), 1.08 (t, 3H, |
| | CH₃) |
| 398 | ¹H NMR δ (ppm) (DMSO-d₆): 8.47 (s, 1H, ArH), 7.97 (s, 1H, ArH), 7.25 (d, 1H, |
| | ArH), 7.17-7.21 (m, 1H, ArH), 7.11 (s, 1H, ArH), 7.13 (t, 1H, CH), 6.19 (s, 1H, |
| | ArH), 5.05 (br. s., 1H, NH), 3.90 (d, 1H, CH), 3.63 (d, 1H, CH), 3.60 (s, 3H, CH₃), |
| | 3.57 (d, 2H, CH₂), 3.55-3.60 (m, 2H, CH₂), 3.31 (br. s., 3H, CH₃), 2.50 (s, 2H, CH₂), |
| | 2.44 (q, 1H, CH), 2.22 (dt, 1H, CH), 1.08 (t, 3H, CH₃) |
| 399 | ¹H NMR δ (ppm) (DMSO-d₆): 8.45 (d, 1H, ArH), 7.96 (s, 1H, ArH), 7.87 (s, 1H, |
| | ArH), 6.39 (s, 1H, ArH), 6.16 (s, 1H, ArH), 4.17 (t, 2H, CH₂), 4.03 (dd, 2H, CH₂), |
| | 3.68 (s, 3H, CH₃), 3.60-368 (m, 1H, CH), 3.60 (s, 3H, CH₃), 3.30 (s, 3H, CH₃), |
| | 2.30 (q, 2H, CH₂), 1.05 (t, 3H, CH₃) |
| 400 | ¹H NMR δ (ppm) (DMSO-d₆): 8.46 (d, 1H, ArH), 7.95 (s, 1H, ArH), 7.94 (br. s, 1H, |
| | NH), 7.86 (s, 1H, ArH), 6.35 (s, 1H, ArH), 6.14 (d, 1H, ArH), 4.09 (t, 2H, CH₂), |
| | 3.96 (t, 2H, CH₂), 3.60 (s, 3H, CH₃), 3.36-3.48 (m, 1H, CH), 3.30 (s, 3H, CH₃), 2.31 (q, |
| | 2H, CH₂), 1.05 (t, 3H, CH₃) |
| 401 | ¹H NMR δ (ppm) (DMSO-d₆): 8.78 (t, 1H, NH), 8.46 (d, 1H, ArH), 7.96 (s, 1H, |
| | ArH), 7.87 (s, 1H, ArH), 6.38 (s, 1H, ArH), 6.16 (s, 1H, ArH), 4.18 (d, 2H, CH₂), |
| | 4.11 (t, 2H, CH₂), 3.98 (dd, 2H, CH₂), 3.60 (s, 3H, CH₃), 3.30 (s, 3H, CH₃), 3.32 (q, |
| | 2H, CH₂), 1.05 (t, 3H, CH₃) |
| 402 | ¹H NMR δ (ppm) (CDCl₃): 8.71 (d, 1H, ArH), 8.02 (s, 1H, ArH), 7.63 (s, 2H, ArH), |
| | 6.31 (s, 1H, ArH), 5.91 (d, 1H, ArH), 4.42-4.27 (m, 4H, CH₂), 3.66-3.60 (m, 1H, |
| | CH), 3.59 (s, 3H, CH₃), 3.43 (s, 3H, CH₃), 2.45 (q, 2H, CH₂), 1.14 (t, 3H, CH₃) |
| 403 | ¹H NMR δ (ppm) (DMSO-d₆): 8.43 (s, 1H, ArH), 8.10 (s, 1H, ArH), 7.88 (s, 1H, |
| | ArH), 7.84 (s, 1H, ArH), 6.96 (s, 1H, ArH), 3.77 (s, 3H, CH₃), 3.36 (s, 3H, CH₃) |
| 404 | ¹H NMR δ (ppm) (DMSO-d₆): 8.42 (s, 1H, ArH), 8.05 (s, 1H, ArH), 7.69 (t, 1H, NH), |
| | 7.16 (s, 1H, ArH), 7.14 (s, 1H, ArH), 6.71 (s, 1H, ArH), 4.21 (d, 2H, CH₂), 3.73 (s, |
| | 3H, CH₃), 3.30 (s, 3H, CH₃), 2.95 (s, 3H, CH₃) |
| 405 | ¹H NMR δ (ppm) (DMSO-d₆): 8.41 (d, 1H, ArH), 8.06 (s, 1H, ArH), 7.17-7.24 (m, |
| | 2H, ArH), 6.76 (d, 1H, ArH), 4.11 (s, 2H, CH₂), 3.74 (s, 3H, CH₃), 3.31 (s, 3H, CH₃) |
| 407 | ¹H NMR δ (ppm) (DMSO-d₆): 8.43 (s, 1H, ArH), 8.33 (s, 1H, ArH), 8.02 (s, 1H, |
| | ArH), 7.41 (s, 1H, ArH), 6.47 (s, 1H, ArH), 4.20 (s, 2H, CH₂), 3.68 (s, 3H, CH₃), |
| | 3.34 (s, 3H, CH₃), 2.42 (q, 2H, CH₂), 1.08 (t, 3H, CH₃) |
| 409 | ¹H NMR δ (ppm) (D6-DMSO): 8.41 (s, 1H, ArH), 8.05 (s, 1H, ArH), 7.25 (s, 1H, |
| | ArH), 7.22 (s, 1H, ArH), 6.73 (s, 1H, ArH), 4.183 (t, 2H, CH₂), 4.00 (t, 2H, CH₂), |
| | 3.93-4.00 (m, 1H, CH), 3.73 (s, 3H, CH₃), 3.31 (s, 1H, CH₃), 3.09 (s, 3H, CH₃) |
| 410 | Rotamer 1 |
| | ¹H NMR δ (ppm) (CDCl₃): 8.71 (s, 1H, ArH), 7.64 (s, 1H, ArH), 7.52 (s, 1H, ArH), |
| | 7.43 (d, 1H, ArH), 7.28 (s, 1H, ArH), 6.13 (br. s, 1H, NH), 5.93 (s, 1H, ArH), 4.98 (s, |
| | 1H, CH), 3.60 (s, 3H, CH₃), 3.52-3.44 (m, 2H, CH₂), 3.43 (s, 3H, CH₃), 3.30 (s, 2H, |
| | CH₂), 2.99-2.83 (m, 2H, CH₂), 2.55 (q, 2H, CH₂), 1.17 (t, 3H, CH₃) |
| | Rotamer 2 |
| | ¹H NMR δ (ppm) (CDCl₃): 8.71 (s, 1H, ArH), 7.64 (s, 1H, ArH), 7.53 (s, 1H, ArH), |
| | 7.46 (d, 1H, ArH), 7.25 (s, 1H, ArH), 6.13 (br. s, 1H, NH), 5.93 (s, 1H, ArH), 4.98 (s, |
| | 1H, CH), 3.60 (s, 3H, CH₃), 3.52-3.44 (m, 2H, CH₂), 3.43 (s, 3H, CH₃), 3.30 (s, 2H, |
| | CH₂), 2.99-2.83 (m, 2H, CH₂), 2.55 (q, 2H, CH₂), 1.20 (t, 3H, CH₃) |
| 411 | ¹H NMR δ (ppm) (DMSO-d₆): 8.52 (s, 1H, ArH), 8.42 (s, 1H, ArH), 7.99-8.01 (m, |
| | 2H, ArH + NH), 7.08 (s, 1H, ArH), 6.62 (s, 1H, ArH), 3.70 (s, 3H, CH₃), |
| | 3.54-3.65 (m, 1H, CH), 2.60 (q, 2H, CH₂), 2.20-2.32 (m, 4H, CH), 1.91-2.05 (m, 1H, CH), |
| | 1.79-1.84 (m, 1H, CH), 1.12 (t, 3H, CH₃) |
| 412 | ¹H NMR δ (ppm) (DMSO-d₆): 8.47 (d, 1H, ArH), 8.01 (t, 1H, NH), 7.96 (s, 1H, |
| | ArH), 7.87 (s, 1H, ArH), 6.36 (s, 1H, ArH), 6.16 (d, 1H, ArH), 4.07 (t, 2H, CH₂), |
| | 3.97 (t, 2H, CH₂), 3.61 (s, 3H, CH₃), 3.31 (s, 3H, CH₃), 3.16-3.06 (m, 2H, CH₂), 2.33 (q, |
| | 2H, CH₂), 1.06 (t, 3H, CH₃), 1.03 (t, 3H, CH₃) |
| 413 | ¹H NMR δ (ppm) (DMSO-d₆): 8.45 (d, 1H, ArH), 7.98 (s, 1H, ArH), 7.94 (s, 1H, |
| | ArH), 6.57 (s, 1H, ArH), 6.23 (d, 1H, ArH), 4.41 (t, 4H, CH₂), 3.62 (s, 3H, CH₃), |
| | 3.32 (s, 3H, CH₃), 2.36 (q, 2H, CH₂), 1.08 (t, 3H, CH₃) |
| 414 | ¹H NMR δ (ppm) (DMSO-d₆): 8.46 (s, 1H, ArH), 7.95 (s, 1H, ArH), 7.88 (s, 1H, |
| | ArH), 6.46 (s, 1H, ArH), 6.12 (s, 1H, ArH), 4.45 (d, 1H, CH), 3.36-3.80 (m, 6H, CH), |
| | 3.53 (s, 3H, CH₃), 2.35 (q, 2H, CH₂), 2.10-2.35 (m, 2H, CH), 1.06 (t, 3H, CH₃) |
| 415 | ¹H NMR δ (ppm) (DMSO-d₆): 8.47 (d, 1H, ArH), 7.96 (s, 1H, ArH), 7.89 (s, 1H, |
| | ArH), 6.46 (s, 1H, ArH), 6.13 (s, 1H, ArH), 5.46 (dquint., 1H, CH), 3.82-3.29 (m, |
| | 4H, CH₂), 3.59 (s, 3H, CH₃), 3.32 (s, 3H, CH₃), 2.39-2.20 (m, 2H, CH₂), 2.35 (q, 2H, |
| | CH₂), 1.08 (t, 3H, CH₃) |
| 416 | ¹H NMR δ (ppm) (DMSO-d₆): 8.47 (d, 1H, ArH), 7.98 (s, 1H, ArH), 7.93 (s, 1H, |
| | ArH), 6.89 (s, 1H, ArH), 6.20 (s, 1H, ArH), 3.83-3.92 (m, 1H, CH), 3.71-3.80 (m, |
| | 1H, CH), 3.61 (s, 3H, CH₃), 3.56-3.66 (m, 1H, CH), 3.44-3.53 (m, 1H, CH), 3.30 (s, |
| | 3H, CH₃), 3.03-3.12 (m, 1H, CH), 2.35 (q, 2H, CH₂), 1.89-1.98 (m, 2H, 2xCH), |
| | 1.66-1.77 (m, 1H, CH), 1.54-1.65 (m, 1H, CH), 1.08 (t, 3H, CH₃) |
| 417 | ¹H NMR δ (ppm) (DMSO-d₆): 8.47 (d, 1H, ArH), 7.95 (s, 1H, ArH), 7.85 (s, 1H, |
| | ArH), 6.37 (s, 1H, ArH), 6.11 (d, 1H, ArH), 3.60-3.67 (m, 1H, CH), 3.58 (s, 3H, |
| | CH₃), 3.49-3.57 (m, 1H, CH), 3.35-3.42 (m, 1H, CH), 3.31 (s, 3H, CH₃), |
| | 2.91-2.99 (m, 1H, CH), 2.27-2.43 (m, 3H, CH + CH₂), 2.04-2.16 (m, 1H, CH), 1.51-1.66 (m, |
| | 1H, CH), 1.03-1.12 (m, 6H, 2xCH₃) |
| 418 | ¹H NMR δ (ppm) (DMSO-d₆): 8.47 (d, 1H, ArH), 7.96 (s, 1H, ArH), 7.90 (s, 1H, |
| | ArH), 6.49 (s, 1H, ArH), 6.15 (d, 1H, ArH), 3.66-3.79 (m, 2H, 2xCH), 3.47-3.62 (m, |
| | 6H, 3xCH + CH₃), 3.32 (s, 3H, CH₃), 2.21-2.45 (m, 4H, 2xCH + CH₂), 1.08 (t, 3H, |
| | CH₃) |
| 419 | ¹H NMR δ (ppm) (CDCl₃): 8.69 (1 H, s, ArH), 7.59 (1 H, s, ArH), 7.23 (1 H, s, ArH), |
| | 7.16-7.11 (2H, m, ArH), 5.85 (1 H, s, ArH), 4.76 (4H, s, CH₂), 3.57 (2H, s, CH₂), |
| | 3.53 (3H, s, CH₃), 3.45 (4H, s, CH₂), 3.41 (3H, s, CH₃), 2.50 (2H, q, CH₂), 1.13 (3H, |
| | t, CH₃) |
| 420 | ¹H NMR δ (ppm) (CDCl₃): 8.71 (1 H, s, ArH), 7.61 (1 H, s, ArH), 7.30 (1 H, s, ArH), |
| | 7.20-7.23 (1H, m, ArH), 7.14 (1H, d, ArH), 5.86 (1 H, s, ArH), 3.73 (1H, d, CH), |
| | 3.60-3.64 (2H, m, CH₂), 3.58 (3H, s, CH₃), 3.44 (3H, s, CH₃), 3.02-3.05 (1H, m, |
| | CH), 2.93 (1H, d, CH), 2.86 (3H, s, CH₃), 2.83-2.85 (1H, m, CH), 2.69 (1H, q, CH), |
| | 2.51 (2H, q, CH₂), 2.25-2.35 (2H, m, CH₂), 1.14 (3H, t, CH₃). |
| 421 | ¹H NMR δ (ppm) (CDCl₃): 8.70 (1 H, s, ArH), 7.60 (1 H, s, ArH), 7.31 (1 H, s, ArH), |
| | 7.22-7.25 (1H, m, ArH), 7.14 (1H, d, ArH), 6.51 (1 H, b, NH), 5.87 (1 H, s, ArH), |
| | 3.61 (2H, s, CH₂), 3.55 (3H, s, CH₃), 3.42 (5H, s, CH₃ + CH₂), 3.17 (2H, s, CH₂), |
| | 2.74 (2H, t, CH₂), 2.50 (2H, q, CH₂), 1.14 (3H, t, CH₃) |
| 422 | ¹H NMR δ (ppm) (CDCl₃): 8.71 (1 H, s, ArH), 7.60 (1 H, s, ArH), 7.30 (1 H, s, ArH), |
| | 7.22-7.25 (1H, m, ArH), 7.14 (1H, d, ArH), 5.87 (1 H, s, ArH), 3.58 (2H, s, CH₂), |
| | 3.54 (3H, s, CH₃), 3.43 (3H, s, CH₃), 2.58 (4H, s, CH₂), 2.52 (2H, q, CH₂), |
| | 2.07-2.00 (4H, m, CH₂), 1.14 (3H, t, CH₃) |
| 423 | ¹H NMR δ (ppm) (CDCl₃): 8.69 (1 H, s, ArH), 7.68 (1 H, s, ArH), 7.37 (1 H, s, ArH), |
| | 7.31-7.28 (1 H, m, ArH), 7.19 (1 H, d, ArH), 5.94 (1 H, s, ArH), 4.72 (1 H, b, NH), |
| | 4.35 (2H, s, CH₂), 3.60 (3H, s, CH₃), 3.42 (3H, s, CH₃), 2.52 (2H, q, CH₂), 1.15 (3H, |
| | t, CH₃) |
| 424 | ¹H NMR δ (ppm) (CDCl₃): 8.67 (1 H, s, ArH), 7.62 (1 H, s, ArH), 7.34 (1 H, s, ArH), |
| | 7.24-7.27 (1H, m, ArH), 7.20 (1H, d, ArH), 5.91 (1 H, s, ArH), 4.93 (1H, b, NH), |
| | 4.34 (2H, s, CH₂), 4.21 (4H, t, CH₂), 3.57 (3H, s, CH₃), 3.42 (3H, s, CH₃), 2.52 (2H, |
| | q, CH₂), 1.14 (3H, t, CH₃) |
| 425 | ¹H NMR δ (ppm) (CDCl₃): 8.68 (1 H, s, ArH), 7.62 (1 H, s, ArH), 7.34 (1 H, s, ArH), |
| | 7.25-7.28 (1H, m, ArH), 7.20 (1H, d, ArH), 5.92 (1 H, s, ArH), 4.29 (2H, s, CH₂), |
| | 3.51-3.63 (7H, m, CH₂ + CH₃ + CH₂), 3.41 (3H, s, CH₃), 3.52 (2H, q, CH₂), |
| | 2.30-2.41 (2H, m, CH₂), 1.14 (3H, t, CH₃). |
| 426 | ¹H NMR δ (ppm) (CDCl₃): 8.72 (1 H, s, ArH), 7.65 (1 H, s, ArH), 7.36 (1 H, s, ArH), |
| | 7.29-7.31 (1 H, m, ArH), 7.22 (1 H, d, ArH), 5.94 (1 H, s, ArH), 4.75 (1 H, t, NH), |
| | 4.31 (2H, d, CH₂), 3.72-3.74 (4 H, m, CH₂), 3.60 (3H, s, CH₃), 3.44 (3H, s, CH₃), |
| | 3.23-3.26 (4H, m, CH₂), 2.55 (2H, q, CH₂), 1.16 (3H, t, CH₃). |
| 427 | ¹H NMR δ (ppm) (DMSO-d₆): 8.44 (s, 1H, ArH), 8.00 (s, 1H, ArH), 7.23 (s, 1H, |
| | ArH), 7.13 (s, 1H, ArH), 7.11 (s, 1H, ArH), 6.98 (t, 1H, CH), 6.42 (s, 1H, ArH), |
| | 3.65 (s, 3H, CH₃), 2.35 (s, 3H, CH₃) |
| 428 | ¹H NMR δ (ppm) (CDCl₃): 8.67 (d, 1H, ArH), 7.76 (s, 1H, ArH), 7.56, (dd, 1H, ArH), |
| | 7.52 (br. s, 1H, ArH), 7.41 (d, 1H, 1rH), 6.55 (d, 1H, ArH), 6.34 (t, 1H, CH), 3.75 (s, |
| | 3H, CH₃), 3.48 (s, 3H, CH₃) |
| 430 | ¹H NMR δ (ppm) (CDCl₃): 8.67 (s, 1H, ArH), 7.69 (s, 1H, ArH), 7.25-7.37 (m, 3H), |
| | 6.40 (t, 1H, CH), 6.32 (s, 1H, CH), 4.34 (s, 2H, CH₂), 3.69 (s, 3H, CH₃), 3.43 (s, 3H, |
| | CH₃), 2.90 (s, 3H, CH₃), 2.87 (s, 3H, CH₃) |
| 431 | ¹H NMR δ (ppm) (CDCl₃): 8.75 (1 H, d, ArH), 7.87 (1 H, s, ArH), 7.40 (1 H, dd, |
| | ArH), 7.35-7.29 (m, 2H, ArH), 7.18 (1 H, dd, ArH), 7.04 (1 H, t, CHF2), 6.02 (1 H, s, |
| | ArH), 3.45 (3 H, t, CH₃), 2.52 (2H, q, CH₂), 1.15 (3 H, t, CH₃) |
| 432 | ¹H NMR δ (ppm) (DMSO-d₆): 8.44 (d, 1H, ArH), 8.23 (s, 1H, ArH), 8.00 (s, 1H, |
| | ArH), 7.22 (s, 1H, ArH), 6.38 (d, 1H, ArH), 3.66 (s, 3H, CH₃), 2.73-2.62 (m, 1H, |
| | CH), 2.39 (q, 2H, CH₂), 1.94-1.67 (m, 5H, CH₂), 1.61-1.20 (m, 5H, CH₂), 1.08 (t, |
| | 3H, CH₃) |
| 434 | ¹H NMR δ (ppm) (DMSO-d₆): 8.43 (s, 1H, ArH), 8.27 (s, 1H, ArH), 7.99 (s, 1H, |
| | ArH), 6.35 (s, 1H, ArH), 3.59-3.71 (m, 1H, CH), 3.65 (s, 3H, CH₃), 2.38 (q, 2H, |
| | CH₂), 2.22-2.33 (m, 4H, CH), 1.95-2.07 (m, 1H, CH), 1.76-1.90 (m, 1H, CH), 1.07 (t, |
| | 3H, CH₃) |
| 435 | ¹H NMR δ (ppm) (DMSO-d₆): 8.47 (s, 1H, ArH), 7.99 (s, 1H, ArH), 7.42 (s, 1H, |
| | ArH), 7.36 (d, 1H, ArH), 7.25 (d, 1H, ArH), 6.24 (s, 1H, ArH), 5.43 (s, 1H, CHCN), |
| | 3.62 (s, 3H, CH₃), 3.35-3.30 (m, 3H, CH₃), 2.98 (br. s, 1H, CHCN), 2.73 (br. s, 1H, |
| | CH₂), 2.63-2.56 (m, 1H, CH₂), 2.48 (s, 2H, CH₂), 2.47-2.44 (m, 2H, CH₂), |
| | 1.97-1.89 (m, 2H, CH₂), 1.86-1.79 (m, 1H, CH₂), 1.74 (d, 1H, CH₂), 1.09 (t, 3H, CH₃) |
| 436 | ¹H NMR δ (ppm) (DMSO-d₆): 8.46 (s, 1H, ArH), 7.99 (s, 1H, ArH), 7.52 (d, 1H, |
| | NH), 7.37 (s, 1H, ArH), 7.31 (dd, 1H, ArH), 7.22 (d, 1H, ArH), 6.23 (s, 1H, ArH), |
| | 5.15 (s, 1H, CH), 4.04-4.12 (m, 1H, CH), 3.62 (s, 3H, CH₃), 3.53 (t, 1H, CH), |
| | 3.44 (t, 1H, CH), 3.30-3.35 (m, 3H, CH₃), 3.12 (q, 2H, 2xCH), 2.45 (q, 2H, CH₂), |
| | 1.38 (s, 9H, 3xCH₃), 1.08 (t, 3H, CH₃) |
| 437 | ¹H NMR δ (ppm) (DMSO-d₆): 8.46 (d, 1H, ArH), 7.98 (s, 1H, ArH), 7.41 (d, 1H, |
| | ArH), 7.35 (dd, 1H, ArH), 7.23 (d, 1H, ArH), 6.22 (s, 1H, ArH), 5.36 (s, 1H, CH), |
| | 4.44 (t, 1H, CH), 3.60 (s, 3H, CH₃), 3.25 (t, 3H, CH₃), 3.01-3.04 (m, 1H, CH), |
| | 2.58-2.60 (m, 1H, CH), 2.45 (q, 2H, CH₂), 2.26-2.36 (m, 1H, CH), 1.96-2.04 (m, 1H, CH), |
| | 1.63-1.77 (m, 2H, CH₂), 1.20-1.45 (m, 2H, CH + OH), 1.08 (t, 3H, CH₃) |
| 442 | ¹H NMR δ (ppm) (DMSO-d₆): 8.46 (d, 1H, ArH), 7.97 (s, 1H, ArH), 7.85 (s, 1H, |
| | ArH), 6.33 (s, 1H, ArH), 6.14 (d, 1H, ArH), 4.14 (t, 2H, CH₂), 3.86 (quint., 1H, CH), |
| | 3.60 (s, 3H, CH₃), 3.61-3.55 (m, 2H, CH₂), 3.31 (s, 3H, CH₃), 2.95 (br. s, 2H, NH2), |
| | 2.32 (q, 2H, CH₂), 1.05 (t, 3H, CH₃) |
| 443 | ¹H NMR δ (ppm) (DMSO-d₆): 8.45 (d, 1H, ArH), 7.96 (s, 1H, ArH), 7.87 (s, 1H, |
| | ArH), 7.82 (d, 1H, ArH), 6.38 (s, 1H, ArH), 6.16 (d, 1H, NH), 4.38-4.22 (m, 2H, |
| | CH₂), 3.86-3.78 (m, 2H, CH₂), 3.60 (s, 3H, CH₃), 3.30 (s, 3H, CH₃), 2.94 (s, 3H, |
| | CH₃), 2.33 (q, 2H, CH₂), 1.05 (t, 3H, CH₃) |
| 444 | ¹H NMR δ (ppm) (DMSO-d₆): 8.44 (s, 1H, ArH), 8.37 (s, 1H, ArH), 8.04 (s, 1H, |
| | ArH), 7.51 (s, 1H, ArH), 6.53 (s, 1H, ArH), 5.52 (s, 1H, CH), 3.70 (s, 3H, CH₃), |
| | 3.69-3.63 (m, 3H, CH₂), 3.36 (s, 3H, CH₃), 2.67-2.58 (m, 2H, CH₂), 2.57-2.52 (m, 3H, |
| | CH₂), 2.51-2.41 (m, 2H, CH₂), 1.09 (t, 3H, CH₃) |
| 445 | ¹H NMR δ (ppm) (DMSO-d₆): 8.46 (s, 1H, ArH), 7.98 (s, 1H, ArH), 7.43 (dd, 1H, |
| | ArH), 7.38-7.33 (m, 1H, ArH), 7.25 (dd, 1H, ArH), 6.23 (d, 1H, ArH), 5.61 (d, 1H, |
| | CH), 3.61 (s, 3H, CH₃), 3.42-3.35 (m, 1H, CH), 3.32 (s, 3H, CH₃), 3.05-2.98 (m, |
| | 1H, CH₂), 2.97-2.91 (m, 1H, CH₂), 2.90-2.82 (m, 1H, CH₂), 2.82-2.78 (m, 1H, |
| | CH₂), 2.77-2.69 (m, 1H, CH₂), 2.66-2.54 (m, 1H, CH₂), 2.48-2.42 (m, 2H, CH₂), |
| | 2.32-2.20 (m, 1H, CH₂), 2.09-1.96 (m, 1H, CH₂), 1.08 (t, 3H, CH₃) |
| 446 | ¹H NMR δ (ppm) (CDCl₃): 8.70 (s, 1H, ArH), 7.60 (s, 1H, ArH), 7.43 (d, 1H, ArH), |
| | 7.35 (dd, 1H, ArH), 7.19 (d, 1H, ArH), 5.86 (s, 1H, ArH), 4.73 (s, 1H, CH), 3.56 (s, |
| | 3H, CH₃), 3.41 (s, 3H, CH₃), 3.15 (q, 4H, 2xCH₂), 2.51 (q, 2H, CH₂), 1.27 (s, 6H, |
| | 2xCH₃), 1.14 (t, 3H, CH₃) |
| 447 | ¹H NMR δ (ppm) (CDCl₃): 8.70 (s, 1H, ArH), 7.61 (s, 1H, ArH), 7.51 (d, 1H, ArH), |
| | 7.44 (dd, 1H, ArH), 7.21 (d, 1H, ArH), 5.89 (s, 1H, ArH), 4.87 (s, 1H, CH), |
| | 3.76-3.82 (m, 1H, CH), 3.56 (s, 3H, CH₃), 3.42 (s, 3H, CH₃), 2.85-2.91 (m, 1H, CH), |
| | 2.71-2.79 (m, 1H, CH), 2.49-2.63 (m, 3H, CH + CH₂), 2.27-2.36 (m, 1H, CH), 1.90-2.00 (m, |
| | 2H, CH), 1.48-1.77 (m, 3H, CH), 1.15 (t, 3H, CH₃) |
| 448 | ¹H NMR δ (ppm) (CDCl₃): 8.70 (s, 1H, ArH), 7.60 (s, 1H, ArH), 4.50 (s, 1H, ArH), |
| | 7.42 (dd, 1H, ArH), 7.21 (d, 1H, ArH), 5.87 (s, 1H, ArH), 5.00 (d, 1H, CH), |
| | 3.96-4.01 (m, 1H, CH), 3.56 (s, 3H, CH₃), 3.42 (s, 3H, CH₃), 3.28 (d, 3H, CH₃), 2.60-3.01 (m, |
| | 4H, CH), 2.52 (q, 2H, CH₂), 2.11-2.21 (m, 1H, CH), 2.02-2.11 (m, 1H, CH), 1.14 (t, |
| | 3H, CH₃) |
| 449 | ¹H NMR δ (ppm) (DMSO-d₆): 8.46 (d, 1H, ArH), 7.99 (s, 1H, ArH), 7.41 (d, 1H, |
| | ArH), 7.36 (dd, 1H, ArH), 7.25 (d, 1H, ArH), 6.24 (d, 1H, ArH), 5.41 (br. s, 1H, CH), |
| | 3.62 (s, 3H, CH₃), 3.32 (s, 6H, CH₃), 2.51-2.43 (s, 6H, CH₂), 2.23 (br. s, 4H, CH₂), |
| | 1.09 (t, 3H, CH₃) |
| 450 | ¹H NMR δ (ppm) (CDCl₃): 8.71 (s, 1H, ArH), 7.63 (s, 1H, ArH), 7.44 (d, 1H, ArH), |
| | 7.37 (dd, 1H, ArH), 7.23 (d, 1H, ArH), 5.89 (s, 1H, ArH), 4.79 (s, 1H, CH), 4.53 (br. |
| | s, 1H, CH), 3.77 (t, 1H, CH₂), 3.64 (t, 1H, CH₂), 3.58 (s, 3H, CH₃), 3.43 (s, 3H, CH₃), |
| | 3.27 (q, 2H, CH₂), 2.54 (q, 2H, CH₂), 2.31 (br. s, 1H, OH), 1.16 (t, 3H, CH₃) |
| 451 | ¹H NMR δ (ppm) (DMSO-d₆): 8.48 (d, 1H, ArH), 7.99 (s, 1H, ArH), 7.43 (d, 1H, |
| | ArH), 7.37 (dd, 1H, ArH), 7.26 (d, 1H, ArH), 7.25 (br. s, 1H, NH), 6.77 (br. s, 1H, |
| | NH), 6.24 (d, 1H, ArH), 5.39 (br. s, 1H, CH), 3.62 (s, 3H, CH₃), 3.32 (s, 3H, CH₃), |
| | 3.09-3.01 (m, 1H, CH₂), 2.66-2.59 (m, 1H, CH₂), 2.42 (q, 2H, CH₂), 2.40-2.30 (m, |
| | 1H, CH), 2.19-2.00 (m, 2H, CH₂), 1.85-1.65 (m, 3H, CH₂), 1.57-1.42 (m, 1H, |
| | CH₂), 1.09 (t, 3H, CH₃) |
| 452 | ¹H NMR δ (ppm) (DMSO-d₆): 8.47 (d, 1H, ArH), 7.99 (s, 1H, ArH), 7.43 (d, 1H, |
| | ArH), 7.38 (dd, 1H, ArH), 7.26 (d, 1H, ArH), 6.25 (d, 1H, ArH), 5.46 (br. s, 1H, CH), |
| | 3.62 (s, 3H, CH₃), 3.33 (s, 3H, CH₃), 3.25-3.17 (m, 2H, CH₂), 2.94 (s, 3H, CH₃), |
| | 2.77-2.70 (m, 1H, CH), 2.52-2.48 (m, 2H, CH₂), 2.17-2.00 (m, 4H, CH₂), |
| | 1.81-1.70 (m, 1H, CH₂), 1.61-1.47 (m, 2H, CH₂), 1.09 (t, 3H, CH₃) |
| 453 | ¹H NMR δ (ppm) (CDCl₃): 8.72 (d, 1H, ArH), 7.62 (s, 1H, ArH), 7.44 (d, 1H, ArH), |
| | 7.37 (dd, 1H, ArH), 7.23 (d, 1H, ArH), 5.88 (d, 1H, ArH), 4.72 (s, 1H, CH), 4.20 (q, |
| | 2H, CH₂), 3.69-3.55 (m, 4H, CH₂), 3.58 (s, 3H, CH₃), 3.43 (s, 3H, CH₃), |
| | 3.34 (quint., 1H, CH), 2.54 (q, 2H, CH₂), 1.29 (t, 3H, CH₃), 1.16 (t, 3H, CH₃) |
| 454 | ¹H NMR δ (ppm) (DMSO-d₆): 8.46 (d, 1H, ArH), 8.37 (d, 1H, ArH), 7.99 (s, 1H, |
| | ArH), 7.37 (d, 1H, ArH), 7.31 (dd, 1H, ArH), 7.23 (d, 1H, ArH), 6.23 (br. s, 1H, NH), |
| | 5.21 (s, 1H, CH), 4.30 (q, 1H, CH), 3.62 (s, 3H, CH₃), 3.60 (t, 1H, CH₂), 3.47 (t, 1H, |
| | CH₂), 3.32 (s, 3H, CH₃), 3.11 (t, 2H, CH₂), 2.45 (q, 2H, CH₂), 2.08 (q, 2H, CH₂), |
| | 1.08 (t, 3H, CH₃), 0.98 (t, 3H, CH₃) |
| 455 | ¹H NMR δ (ppm) (DMSO-d₆): 8.46 (s, 1H, ArH), 7.99 (s, 1H, ArH), 7.79 (d, 1H, |
| | ArH), 7.38 (d, 1H, ArH), 7.32 (dd, 1H, ArH), 7.23 (d, 1H, ArH), 6.24 (br. s, 1H, NH), |
| | 5.19 (s, 1H, CH), 4.30 (sext., 1H, CH), 3.63 (t, 1H, CH₂), 3.62 (s, 3H, CH₃), 3.54 (t, |
| | 1H, CH₂), 3.32 (s, 3H, CH₃), 3.18 (t, 1H, CH₂), 3.17 (t, 1H, CH₂), 2.89 (s, 3H, CH₃), |
| | 2.46 (q, 2H, CH₂), 1.09 (t, 3H, CH₃) |
| 456 | ¹H NMR δ (ppm) (DMSO-d₆): 9.01 (s, 1H, ArH), 8.59 (s, 1H, ArH), 8.43 (d, 1H, ArH), |
| | 8.06 (s, 1H, ArH), 6.68 (d, 1H, ArH), 3.73 (s, 3H, CH₃), 3.38 (s, 3H, CH₃), 2.53 (q, 2H, |
| | CH₂), 1.13 (t, 3H, CH₃) |
| 457 | ¹H NMR δ (ppm) (CDCl₃): 8.71 (s, 1H, ArH), 8.17 (s, 1H, ArH), 7.77 (s, 1H, ArH), |
| | 6.46 (s, 1H, ArH), 4.35-4.22 (m, 4H, CH₂), 3.98-3.86 (m, 1H, CH), 3.75 (s, 3H, |
| | CH₃), 3.56 (s, 3H, CH₃), 2.51 (q, 2H, CH₂), 1.48 (s, 9H, CH₃), 1.18 (t, 3H, CH₃) |
| 458 | ¹H NMR δ (ppm) (CDCl₃): 8.69 (s, 1H, ArH), 8.49 (s, 1H, ArH), 7.72 (br. s, 1H, |
| | ArH), 7.61 (s, 1H, ArH), 6.14 (s, 1H, ArH), 4.73 (d, 2H, CH₂), 4.48 (d, 2H, CH₂), |
| | 3.69 (s, 3H, CH₃), 3.46 (s, 3H, CH₃), 3.06 (s, 3H, CH₃), 2.56 (q, 2H, CH₂), 1.20 (t, |
| | 3H, CH₃) |
| 459 | ¹H NMR δ (ppm)(DMSO-d₆): 8.55 (1 H, d, ArH), 7.99 (1 H, s, ArH), 7.52-7.43 (1 H, |
| | m, ArH), 7.41-7.27 (3 H, m, ArH), 5.90 (1 H, d, ArH), 3.55 (3 H, s, CH₃), 2.82 (2 H, |
| | t, CH₂), 2.60 (6 H, s, CH₂), 2.48 (2 H, q, CH₂), 1.71 (4 H, s, CH₂), 1.19-1.07 (3 H, m, |
| | CH₃). |
| 460 | ¹H NMR δ (ppm)(DMSO-d₆): 8.55 (1 H, s, ArH), 7.99 (1 H, s, ArH), 7.50-7.45 (1 H, |
| | m, ArH), 7.40-7.30 (3 H, m, ArH), 5.88 (1 H, s, ArH), 3.55 (4 H, s, CH₂), 3.36 (3 H, |
| | s, CH₃), 2.72-2.65 (4 H, m, CH₂), 2.53-2.44 (2 H, m, CH₂), 2.40 (4 H, s, CH₂), |
| | 1.21-1.09 (3 H, m, CH₃). |
| 462 | ¹H NMR δ (ppm)(DMSO-d₆): 8.60 (1 H, d, ArH), 8.50 (1 H, s, ArH), 8.45 (1 H, d, |
| | ArH), 8.03 (1 H, s, ArH), 7.77-7.70 (1 H, m, ArH), 7.45 (1 H, dd, ArH), 7.38-7.28 (3 |
| | H, m, ArH), 7.14 (1 H, dd, ArH), 5.97 (1 H, d, ArH), 5.20 (2 H, s, CH₂), 3.55 (3 H, s, |
| | CH₃), 2.40 (2 H, q, CH₂), 1.01 (3 H, t, CH₃). |
| 463 | ¹H NMR δ (ppm)(DMSO-d₆): 8.63 (1 H, s, ArH), 8.26 (1 H, s, ArH), 7.48 (1 H, d, |
| | ArH), 7.43-7.34 (2 H, m, ArH), 7.29 (1 H, d, ArH), 6.40 (1 H, t, CH), 6.01 (1 H, s, |
| | ArH), 4.25 (2 H, s, CH₂), 3.57 (3 H, s, CH₃), 2.44 (2 H, q, CH₂), 1.09 (3 H, t, CH₃). |

### Biological Examples

### Example 1

### In-Vitro Assays

### 1.1 JAK1 Inhibition Assay

1.1.1 JAK1 Assay polyGT Substrate

Recombinant human JAK1 catalytic domain (amino acids 850-1154; catalog number 08-144) was purchased from Carna Biosciences. 10 ng of JAK1 is incubated with 12.5 μg polyGT substrate (Sigma catalog number P0275) in kinase reaction buffer (15 mM Tris-HCl pH 7.5, 1 mM DTT, 0.01% Tween-20, 10 mM MgCl₂, 2 μM non-radioactive ATP, 0.25 μCi ³³P-gamma-ATP (GE Healthcare, catalog number AH9968) final concentrations) with or without 5 μL containing test compound or vehicle (DMSO, 1% final concentration), in a total volume of 25 μL, in a polypropylene 96-well plate (Greiner, V-bottom). After 45 min at 30° C., reactions are stopped by adding of 25 μL/well of 150 mM phosphoric acid. All of the terminated kinase reaction is transferred to prewashed (75 mM phosphoric acid) 96 well filter plates (Perkin Elmer catalog number 6005177) using a cell harvester (Perkin Elmer). Plates are washed 6 times with 300 μL per well of a 75 mM phosphoric acid solution and the bottom of the plates is sealed. 40 μL/well of Microscint-20 is added, the top of the plates is sealed and readout is performed using the Topcount (Perkin Elmer). Kinase activity is calculated by subtracting counts per min (cpm) obtained in the presence of a positive control inhibitor (10 μM staurosporine) from cpm obtained in the presence of vehicle. The ability of a test compound to inhibit this activity is determined as:

Percentage inhibition=((cpm determined for sample with test compound present−cpm determined for sample with positive control inhibitor)divided by(cpm determined in the presence of vehicle−cpm determined for sample with positive control inhibitor))*100.

Dose dilution series are prepared for the compounds enabling the testing of dose-response effects in the JAK1 assay and the calculation of the IC₅₀ for each compound. Each compound is routinely tested at concentration of 20 μM followed by a 1/3 serial dilution, 8 points (20 μM—6.67 μM—2.22 μM—740 nM—247 nM—82 nM—27 nM—9 nM) in a final concentration of 1% DMSO. When potency of compound series increased, more dilutions are prepared and/or the top concentration was lowered (e.g. 5 μM, 1 μM).

### 1.1.2 JAK1 Ulight-JAK1 Peptide Assay

Recombinant human JAK1 (catalytic domain, amino acids 866-1154; catalog number PV4774) was purchased from Invitrogen. 1 ng of JAK1 was incubated with 20 nM Ulight-JAK1(tyr1023) peptide (Perkin Elmer catalog number TRF0121) in kinase reaction buffer (25 mM MOPS pH6.8, 0.01% Brij-35, 5 mM MgCl₂, 2 mM DTT, 7 μM ATP) with or without 4 μL containing test compound or vehicle (DMSO, 1% final concentration), in a total volume of 20 μL, in a white 384 Opti plate (Perkin Elmer, catalog number 6007290). After 60 min at room temperature, reactions were stopped by adding 20 μL/well of detection mixture (1× detection buffer (Perkin Elmer, catalog number CR97-100C), 0.5 nM Europium-anti-phosphotyrosine (PT66) (Perkin Elmer, catalog number AD0068), 10 mM EDTA). Readout is performed using the Envision with excitation at 320 nm and measuring emission at 615 nm (Perkin Elmer). Kinase activity was calculated by subtracting relative fluorescence units (RFU) obtained in the presence of a positive control inhibitor (10 μM staurosporine) from RFU obtained in the presence of vehicle. The ability of a test compound to inhibit this activity was determined as:

Percentage inhibition=((RFU determined for sample with test compound present−RFU determined for sample with positive control inhibitor)divided by(RFU determined in the presence of vehicle−RFU determined for sample with positive control inhibitor))*100.

Dose dilution series were prepared for the compounds enabling the testing of dose-response effects in the JAK1 assay and the calculation of the IC50 for the compound. Each compound is routinely tested at concentration of 20 μM followed by a 1/5 serial dilution, 10 points in a final concentration of 1% DMSO. When potency of compound series increases, more dilutions are prepared and/or the top concentration are lowered (e.g. 5 μM, 1 μM). The data are expressed as the average IC50 from the assays±standard error of the mean.

The following compounds have been tested for their activity against JAK1 and the IC₅₀ values, as determined using the assays described herein, are given below in Table III.

TABLE III
JAK1Values of Compounds

| Cpd # | JAK1 |
|---|---|
| 1 | *** |
| 2 | *** |
| 3 | * |
| 4 | *** |
| 5 | *** |
| 6 | ** |
| 7 | * |
| 8 | * |
| 9 | * |
| 10 | * |
| 11 | *** |
| 12 | ** |
| 13 | ** |
| 14 | * |
| 15 | *** |
| 16 | **** |
| 17 | **** |
| 18 | ** |
| 19 | *** |
| 20 | * |
| 21 | ** |
| 22 | *** |
| 23 | ** |
| 24 | *** |
| 25 | * |
| 26 | **** |
| 27 | *** |
| 28 | **** |
| 29 | **** |
| 30 | *** |
| 31 | **** |
| 32 | **** |
| 33 | **** |
| 34 | **** |
| 35 | **** |
| 36 | * |
| 37 | *** |
| 38 | **** |
| 39 | **** |
| 40 | * |
| 41 | * |
| 42 | *** |
| 43 | *** |
| 44 | ** |
| 45 | *** |
| 46 | * |
| 47 | **** |
| 48 | **** |
| 49 | ** |
| 50 | * |
| 51 | * |
| 52 | ** |
| 53 | *** |
| 54 | ** |
| 55 | *** |
| 56 | *** |
| 57 | **** |
| 58 | * |
| 59 | *** |
| 60 | **** |
| 61 | **** |
| 62 | ** |
| 63 | *** |
| 64 | **** |
| 65 | * |
| 66 | *** |
| 67 | *** |
| 68 | *** |
| 69 | *** |
| 70 | *** |
| 71 | **** |
| 72 | *** |
| 73 | **** |
| 74 | ** |
| 75 | ** |
| 76 | * |
| 77 | * |
| 78 | *** |
| 79 | * |
| 80 | **** |
| 81 | **** |
| 82 | **** |
| 83 | **** |
| 84 | **** |
| 85 | **** |
| 86 | **** |
| 87 | *** |
| 88 | *** |
| 89 | *** |
| 90 | *** |
| 91 | **** |
| 92 | **** |
| 93 | **** |
| 94 | **** |
| 95 | **** |
| 96 | * |
| 97 | *** |
| 98 | **** |
| 99 | *** |
| 100 | ** |
| 101 | **** |
| 102 | **** |
| 103 | **** |
| 104 | * |
| 105 | **** |
| 106 | * |
| 107 | **** |
| 108 | *** |
| 109 | *** |
| 110 | **** |
| 111 | **** |
| 112 | * |
| 113 | * |
| 114 | **** |
| 115 | **** |
| 116 | *** |
| 117 | *** |
| 118 | *** |
| 119 | **** |
| 120 | **** |
| 121 | **** |
| 122 | *** |
| 123 | **** |
| 124 | *** |
| 125 | *** |
| 126 | **** |
| 127 | **** |
| 128 | *** |
| 129 | **** |
| 130 | **** |
| 131 | **** |
| 132 | **** |
| 133 | **** |
| 134 | **** |
| 135 | **** |
| 136 | **** |
| 137 | **** |
| 138 | *** |
| 139 | **** |
| 140 | **** |
| 141 | *** |
| 142 | **** |
| 143 | **** |
| 144 | **** |
| 145 | **** |
| 146 | **** |
| 147 | **** |
| 148 | **** |
| 149 | **** |
| 150 | **** |
| 151 | **** |
| 152 | **** |
| 153 | **** |
| 154 | **** |
| 155 | **** |
| 156 | **** |
| 157 | **** |
| 158 | **** |
| 159 | **** |
| 160 | **** |
| 161 | *** |
| 162 | **** |
| 163 | **** |
| 164 | **** |
| 165 | *** |
| 166 | **** |
| 167 | **** |
| 168 | **** |
| 169 | **** |
| 170 | ** |
| 171 | *** |
| 172 | **** |
| 173 | **** |
| 174 | *** |
| 175 | ** |
| 176 | **** |
| 177 | **** |
| 178 | **** |
| 179 | **** |
| 180 | *** |
| 181 | **** |
| 182 | **** |
| 183 | **** |
| 184 | *** |
| 185 | * |
| 186 | **** |
| 187 | **** |
| 188 | *** |
| 189 | **** |
| 190 | **** |
| 191 | **** |
| 192 | **** |
| 193 | **** |
| 194 | **** |
| 195 | **** |
| 196 | **** |
| 197 | **** |
| 198 | **** |
| 199 | **** |
| 200 | **** |
| 201 | **** |
| 202 | **** |
| 203 | **** |
| 204 | **** |
| 205 | **** |
| 206 | **** |
| 207 | **** |
| 208 | **** |
| 209 | **** |
| 210 | **** |
| 211 | **** |
| 212 | **** |
| 213 | **** |
| 214 | **** |
| 215 | **** |
| 216 | **** |
| 217 | **** |
| 218 | **** |
| 219 | **** |
| 220 | **** |
| 221 | **** |
| 222 | **** |
| 223 | **** |
| 224 | **** |
| 225 | **** |
| 226 | **** |
| 227 | **** |
| 228 | **** |
| 229 | **** |
| 230 | **** |
| 231 | **** |
| 232 | **** |
| 233 | **** |
| 234 | **** |
| 235 | **** |
| 236 | **** |
| 237 | **** |
| 238 | **** |
| 239 | **** |
| 240 | **** |
| 241 | **** |
| 242 | **** |
| 243 | **** |
| 244 | **** |
| 245 | **** |
| 246 | **** |
| 247 | **** |
| 248 | **** |
| 249 | **** |
| 250 | **** |
| 251 | **** |
| 252 | **** |
| 253 | **** |
| 254 | **** |
| 255 | **** |
| 256 | **** |
| 257 | **** |
| 258 | **** |
| 259 | **** |
| 260 | **** |
| 261 | **** |
| 262 | **** |
| 263 | ** |
| 264 | **** |
| 265 | ** |
| 266 | * |
| 267 | ** |
| 268 | * |
| 269 | **** |
| 270 | *** |
| 271 | **** |
| 272 | **** |
| 273 | * |
| 274 | **** |
| 275 | * |
| 276 | **** |
| 277 | **** |
| 278 | ** |
| 279 | * |
| 280 | **** |
| 281 | *** |
| 282 | * |
| 283 | **** |
| 284 | **** |
| 285 | *** |
| 286 | *** |
| 287 | **** |
| 288 | **** |
| 289 | **** |
| 290 | **** |
| 291 | **** |
| 292 | **** |
| 293 | **** |
| 294 | **** |
| 295 | **** |
| 296 | ** |
| 297 | ** |
| 298 | ** |
| 299 | **** |
| 300 | **** |
| 301 | **** |
| 302 | **** |
| 303 | ** |
| 304 | **** |
| 305 | * |
| 306 | * |
| 307 | **** |
| 308 | * |
| 309 | * |
| 310 | * |
| 311 | **** |
| 312 | * |
| 313 | **** |
| 314 | * |
| 315 | * |
| 316 | * |
| 317 | **** |
| 318 | *** |
| 319 | **** |
| 320 | **** |
| 321 | * |
| 322 | **** |
| 323 | **** |
| 324 | **** |
| 325 | **** |
| 326 | **** |
| 327 | **** |
| 328 | **** |
| 329 | **** |
| 330 | **** |
| 331 | **** |
| 332 | *** |
| 333 | **** |
| 334 | **** |
| 335 | **** |
| 336 | **** |
| 337 | ** |
| 338 | ** |
| 339 | **** |
| 340 | *** |
| 341 | *** |
| 342 | *** |
| 343 | **** |
| 344 | **** |
| 345 | *** |
| 346 | **** |
| 347 | **** |
| 348 | *** |
| 349 | **** |
| 350 | **** |
| 351 | **** |
| 352 | **** |
| 353 | **** |
| 354 | **** |
| 355 | **** |
| 356 | **** |
| 357 | **** |
| 358 | **** |
| 359 | *** |
| 360 | *** |
| 362 | ** |
| 363 | **** |
| 364 | **** |
| 365 | **** |
| 366 | ** |
| 367 | ** |
| 368 | ** |
| 369 | **** |
| 370 | **** |
| 371 | **** |
| 372 | **** |
| 373 | **** |
| 374 | *** |
| 375 | **** |
| 376 | **** |
| 377 | **** |
| 378 | **** |
| 379 | **** |
| 380 | **** |
| 381 | **** |
| 382 | *** |
| 383 | **** |
| 384 | **** |
| 385 | **** |
| 386 | *** |
| 387 | **** |
| 388 | **** |
| 389 | **** |
| 390 | **** |
| 391 | **** |
| 392 | **** |
| 393 | **** |
| 394 | **** |
| 395 | **** |
| 396 | **** |
| 397 | **** |
| 398 | **** |
| 399 | **** |
| 400 | *** |
| 401 | *** |
| 402 | **** |
| 403 | ** |
| 404 | **** |
| 405 | **** |
| 406 | **** |
| 407 | **** |
| 408 | * |
| 409 | **** |
| 410 | **** |
| 411 | **** |
| 412 | ** |
| 413 | **** |
| 414 | **** |
| 415 | **** |
| 416 | **** |
| 417 | **** |
| 418 | **** |
| 419 | *** |
| 420 | **** |
| 421 | **** |
| 422 | **** |
| 423 | **** |
| 424 | **** |
| 425 | **** |
| 426 | **** |
| 427 | **** |
| 428 | *** |
| 429 | **** |
| 430 | **** |
| 431 | *** |
| 432 | **** |
| 433 | **** |
| 434 | **** |
| 435 | **** |
| 436 | **** |
| 437 | **** |
| 438 | **** |
| 439 | **** |
| 440 | **** |
| 441 | **** |
| 442 | *** |
| 443 | **** |
| 444 | **** |
| 445 | **** |
| 446 | **** |
| 447 | **** |
| 448 | **** |
| 449 | **** |
| 450 | **** |
| 451 | **** |
| 452 | **** |
| 453 | **** |
| 454 | **** |
| 455 | **** |
| 456 | * |
| 457 | * |
| 458 | **** |
| 459 | *** |
| 460 | *** |
| 461 | * |
| 462 | ** |
| 463 | **** |
| 464 | *** |

* >1001 nM
** 501-1000 nM
*** 101-500 nM
**** 0.01-100 nM

### 1.1.3 JAK1 Ki Determination Assay

For the determination of Ki, different amounts of compound are mixed with the enzyme and the enzymatic reaction is followed as a function of ATP concentration. The Ki is determined by means of double reciprocal plotting of Km vs compound concentration (Lineweaver-Burk plot). 1 ng of JAK1 (Invitrogen, PV4774) is used in the assay. The substrate was 50 nM Ulight-JAK-1 (Tyr1023) Peptide (Perkin Elmer, TRF0121) The reaction is performed in 25 mM MOPS pH 6.8, 0.01%, 2 mM DTT, 5 mM MgCl₂ Brij-35 with varying concentrations of ATP and compound. Phosphorylated substrate is measured using an Eu-labeled anti-phosphotyrosine antibody PT66 (Perkin Elmer, AD0068) as described in 1.1.2. Readout is performed on the envision (Perkin Elmer) with excitation at 320 nm and emission followed at 615 nm and 665 nm.

### 1.2 JAK2 Inhibition Assay

1.2.1 JAK2 Assay polyGT Substrate

Recombinant human JAK2 catalytic domain (amino acids 808-1132; catalog number PV4210) was purchased from Invitrogen. 0.025 mU of JAK2 is incubated with 2.5 μg polyGT substrate (Sigma catalog number P0275) in kinase reaction buffer (5 mM MOPS pH 7.5, 9 mM MgAc, 0.3 mM EDTA, 0.06% Brij and 0.6 mM DTT, 1 μM non-radioactive ATP, 0.25 μCi ³³P-gamma-ATP (GE Healthcare, catalog number AH9968) final concentrations) with or without 5 μL containing test compound or vehicle (DMSO, 1% final concentration), in a total volume of 25 μL, in a polypropylene 96-well plate (Greiner, V-bottom). After 90 min at 30° C., reactions are stopped by adding of 25 μL/well of 150 mM phosphoric acid. All of the terminated kinase reaction is transferred to prewashed (75 mM phosphoric acid) 96 well filter plates (Perkin Elmer catalog number 6005177) using a cell harvester (Perkin Elmer). Plates are washed 6 times with 300 μL per well of a 75 mM phosphoric acid solution and the bottom of the plates is sealed. 40 μL/well of Microscint-20 is added, the top of the plates is sealed and readout is performed using the Topcount (Perkin Elmer). Kinase activity is calculated by subtracting counts per min (cpm) obtained in the presence of a positive control inhibitor (10 μM staurosporine) from cpm obtained in the presence of vehicle. The ability of a test compound to inhibit this activity is determined as:

Percentage inhibition=((cpm determined for sample with test compound present−cpm determined for sample with positive control inhibitor)divided by(cpm determined in the presence of vehicle−cpm determined for sample with positive control inhibitor))*100.

Dose dilution series are prepared for the compounds enabling the testing of dose-response effects in the JAK2 assay and the calculation of the IC₅₀ for each compound. Each compound is routinely tested at concentration of 20 μM followed by a 1/3 serial dilution, 8 points (20 μM—6.67 μM—2.22 μM—740 nM—247 nM—82 nM—27 nM—9 nM) in a final concentration of 1% DMSO. When potency of compound series increased, more dilutions are prepared and/or the top concentration is lowered (e.g. 5 μM, 1 μM).

### 1.2.2 JAK2 Ulight-JAK1 Peptide Assay

Recombinant human JAK2 (catalytic domain, amino acids 866-1154; catalog number PV4210) was purchased from Invitrogen. 0.0125 mU of JAK2 was incubated with 25 nM Ulight-JAK1(tyr1023) peptide (Perkin Elmer catalog number TRF0121) in kinase reaction buffer (25 mM HEPES pH7.0, 0.01% Triton X-100, 7.5 mM MgCl₂, 2 mM DTT, 7.5 μM ATP) with or without 4 μL containing test compound or vehicle (DMSO, 1% final concentration), in a total volume of 20 μL, in a white 384 Opti plate (Perkin Elmer, catalog number 6007290). After 60 min at room temperature, reactions were stopped by adding 20 μL/well of detection mixture (1× detection buffer (Perkin Elmer, catalog number CR97-100C), 0.5 nM Europium-anti-phosphotyrosine (PT66) (Perkin Elmer, catalog number AD0068), 10 mM EDTA). Readout is performed using the Envision with excitation at 320 nm and measuring emission at 615 nm (Perkin Elmer). Kinase activity was calculated by subtracting relative fluorescence units (RFU) obtained in the presence of a positive control inhibitor (10 μM staurosporine) from RFU obtained in the presence of vehicle. The ability of a test compound to inhibit this activity was determined as:

Percentage inhibition=((RFU determined for sample with test compound present−RFU determined for sample with positive control inhibitor)divided by(RFU determined in the presence of vehicle−RFU determined for sample with positive control inhibitor))*100.

Dose dilution series are prepared for compound enabling the testing of dose-response effects in the JAK2 assay and the calculation of the IC₅₀ for the compound. Each compound is routinely tested at concentration of 20 μM followed by a 1/5 serial dilution, 10 points in a final concentration of 1% DMSO. When potency of compound series increases, more dilutions are prepared and/or the top concentration are lowered (e.g. 5 μM, 1 μM). The data are expressed as the average IC₅₀ from the assays±standard error of the mean.

The following compounds have been tested for their activity against JAK2 and the IC₅₀ values, as determined using the assays described herein, are given below in Table IV.

TABLE IV
JAK2 IC₅₀ Values of Compounds

| Cpd # | JAK2 |
|---|---|
| 1 | # |
| 2 | # |
| 3 | # |
| 4 | # |
| 5 | # |
| 6 | # |
| 7 | N/A |
| 8 | ### |
| 9 | # |
| 10 | # |
| 11 | # |
| 12 | # |
| 13 | # |
| 14 | # |
| 15 | # |
| 16 | ## |
| 17 | ## |
| 18 | # |
| 19 | # |
| 20 | # |
| 21 | # |
| 22 | ## |
| 23 | # |
| 24 | # |
| 25 | N/A |
| 26 | ### |
| 27 | # |
| 28 | ## |
| 29 | # |
| 30 | # |
| 31 | # |
| 32 | # |
| 33 | N/A |
| 34 | ## |
| 35 | # |
| 36 | N/A |
| 37 | # |
| 38 | # |
| 39 | ## |
| 40 | N/A |
| 41 | N/A |
| 42 | # |
| 43 | # |
| 44 | N/A |
| 45 | # |
| 46 | # |
| 47 | ### |
| 48 | # |
| 49 | # |
| 50 | N/A |
| 51 | # |
| 52 | # |
| 53 | # |
| 54 | N/A |
| 55 | # |
| 56 | # |
| 57 | ### |
| 58 | N/A |
| 59 | # |
| 60 | # |
| 61 | # |
| 62 | # |
| 63 | # |
| 64 | ### |
| 65 | # |
| 66 | # |
| 67 | # |
| 68 | # |
| 69 | # |
| 70 | ## |
| 71 | ### |
| 72 | # |
| 73 | ## |
| 74 | # |
| 75 | ## |
| 76 | # |
| 77 | # |
| 78 | # |
| 79 | # |
| 80 | ### |
| 81 | ### |
| 82 | ### |
| 83 | ### |
| 84 | ### |
| 85 | ## |
| 86 | ## |
| 87 | # |
| 88 | # |
| 89 | # |
| 90 | # |
| 91 | # |
| 92 | # |
| 93 | #### |
| 94 | ### |
| 95 | ### |
| 96 | # |
| 97 | # |
| 98 | ## |
| 99 | # |
| 100 | N/A |
| 101 | #### |
| 102 | # |
| 103 | ### |
| 104 | # |
| 105 | ### |
| 106 | N/A |
| 107 | #### |
| 108 | # |
| 109 | # |
| 110 | ### |
| 111 | ### |
| 112 | N/A |
| 113 | # |
| 114 | ### |
| 115 | ### |
| 116 | # |
| 117 | # |
| 118 | # |
| 119 | # |
| 120 | ### |
| 121 | ## |
| 122 | # |
| 123 | # |
| 124 | # |
| 125 | # |
| 126 | ### |
| 127 | ### |
| 128 | # |
| 275 | # |
| 298 | # |
| 299 | ### |
| 300 | ### |
| 301 | #### |
| 302 | ### |
| 303 | # |
| 304 | #### |
| 305 | # |
| 306 | # |
| 307 | ### |
| 308 | # |
| 309 | # |
| 310 | # |
| 311 | #### |
| 312 | # |
| 313 | ### |
| 314 | # |
| 315 | # |
| 316 | # |
| 317 | ### |
| 318 | ## |
| 319 | #### |
| 320 | #### |
| 321 | # |
| 322 | ### |
| 323 | #### |
| 324 | #### |
| 325 | #### |
| 326 | #### |
| 327 | #### |
| 328 | ### |
| 329 | #### |
| 330 | ### |
| 331 | ### |
| 332 | ## |
| 333 | ### |
| 334 | #### |
| 335 | ## |
| 336 | # |
| 337 | # |
| 338 | # |
| 339 | ### |
| 340 | # |
| 341 | # |
| 342 | # |
| 343 | ### |
| 344 | ### |
| 345 | ## |
| 346 | #### |
| 347 | ### |
| 348 | ## |
| 349 | #### |
| 350 | ### |
| 351 | #### |
| 352 | #### |
| 353 | #### |
| 354 | #### |
| 355 | #### |
| 356 | #### |
| 357 | #### |
| 358 | #### |
| 359 | ### |
| 360 | ### |
| 362 | # |
| 363 | #### |
| 364 | ### |
| 365 | ### |
| 366 | # |
| 367 | # |
| 368 | # |
| 369 | ### |
| 370 | #### |
| 371 | ### |
| 372 | ### |
| 373 | ### |
| 374 | ### |
| 375 | #### |
| 376 | #### |
| 377 | # |
| 378 | ### |
| 379 | ### |
| 380 | #### |
| 381 | ### |
| 382 | # |
| 383 | ### |
| 384 | ### |
| 385 | #### |
| 386 | # |
| 387 | ### |
| 388 | #### |
| 389 | #### |
| 390 | #### |
| 391 | #### |
| 392 | #### |
| 393 | #### |
| 394 | #### |
| 395 | #### |
| 396 | #### |
| 397 | #### |
| 398 | #### |
| 399 | ### |
| 400 | ## |
| 401 | ## |
| 402 | #### |
| 403 | # |
| 404 | #### |
| 405 | #### |
| 406 | #### |
| 407 | #### |
| 408 | # |
| 409 | ### |
| 410 | #### |
| 411 | #### |
| 412 | ## |
| 413 | #### |
| 414 | #### |
| 415 | #### |
| 416 | #### |
| 417 | #### |
| 418 | #### |
| 419 | # |
| 420 | ### |
| 421 | ### |
| 422 | ## |
| 423 | #### |
| 424 | #### |
| 425 | #### |
| 426 | #### |
| 427 | #### |
| 428 | # |
| 429 | #### |
| 430 | #### |
| 431 | # |
| 432 | ### |
| 433 | #### |
| 434 | ### |
| 435 | #### |
| 436 | #### |
| 437 | #### |
| 438 | #### |
| 439 | ### |
| 440 | #### |
| 441 | #### |
| 442 | # |
| 443 | #### |
| 444 | #### |
| 445 | #### |
| 446 | #### |
| 447 | #### |
| 448 | #### |
| 449 | ### |
| 450 | #### |
| 451 | #### |
| 452 | #### |
| 453 | #### |
| 454 | #### |
| 455 | #### |
| 456 | # |
| 457 | # |
| 458 | ### |
| 459 | # |
| 460 | # |
| 461 | N/A |
| 462 | # |
| 463 | ### |
| 464 | # |

N/A not active
# >1001 nM
## 501-1000 nM
### 101-500 nM
#### 0.01-100 nM

### 1.2.3 JAK2 Kd Determination Assay

JAK2 (Invitrogen, PV4210) is used at a final concentration of 5 nM. The binding experiment is performed in 50 mM Hepes pH 7.5, 0.01% Brij-35, 10 mM MgCl₂, 1 mM EGTA using 25 nM kinase tracer 236 (Invitrogen, PV5592) and 2 nM Eu-anti-GST (Invitrogen, PV5594) with varying compound concentrations. Detection of tracer is performed according to the manufacturer's procedure.

### 1.3 JAK3 Inhibition Assay

Recombinant human JAK3 catalytic domain (amino acids 781-1124; catalog number PV3855) was purchased from Invitrogen. 0.5 ng JAK3 protein was incubated with 2.5 μg polyGT substrate (Sigma catalog number P0275) in kinase reaction buffer (25 mM Tris pH 7.5, 0.5 mM EGTA, 10 mM MgCl₂, 2.5 mM DTT, 0.5 mM Na₃VO₄, 5 mM b-glycerolphosphate, 0.01% Triton X-100, 1 μM non-radioactive ATP, 0.25 μCi 33P-gamma-ATP (GE Healthcare, catalog number AH9968) final concentrations) with or without 5 μL containing test compound or vehicle (DMSO, 1% final concentration), in a total volume of 25 μL, in a polypropylene 96-well plate (Greiner, V-bottom). After 45 min at 30° C., reactions were stopped by adding 25 μL/well of 150 mM phosphoric acid. All of the terminated kinase reaction was transferred to prewashed (75 mM phosphoric acid) 96 well filter plates (Perkin Elmer catalog number 6005177) using a cell harvester (Perkin Elmer). Plates were washed 6 times with 300 μL per well of a 75 mM phosphoric acid solution and the bottom of the plates was sealed. 40 μL/well of Microscint-20 was added, the top of the plates was sealed and readout was performed using the Topcount (Perkin Elmer). Kinase activity was calculated by subtracting counts per min (cpm) obtained in the presence of a positive control inhibitor (10 μM staurosporine) from cpm obtained in the presence of vehicle. The ability of a test compound to inhibit this activity was determined as:

Percentage inhibition=((cpm determined for sample with test compound present−cpm determined for sample with positive control inhibitor)divided by(cpm determined in the presence of vehicle−cpm determined for sample with positive control inhibitor))*100.

Dose dilution series were prepared for the compounds enabling the testing of dose-response effects in the JAK3 assay and the calculation of the IC₅₀ for each compound. Each compound was routinely tested at concentration of 20 μM followed by a 1/5 serial dilution, 10 points in a final concentration of 1% DMSO. When potency of compound series increased, more dilutions were prepared and/or the top concentration was lowered (e.g. 5 μM, 1 μM).

The following compounds have been tested for their activity against JAK3 and the IC₅₀ values, as determined using the assays described herein, are given below in Table V.

TABLE V
JAK3 IC₅₀ Values of Compounds

| Cpd # | JAK3 |
|---|---|
| 1 | + |
| 2 | N/A |
| 4 | + |
| 5 | + |
| 16 | ++ |
| 17 | + |
| 26 | + |
| 27 | + |
| 28 | + |
| 29 | + |
| 30 | + |
| 31 | + |
| 32 | + |
| 33 | + |
| 34 | ++ |
| 35 | ++ |
| 38 | ++ |
| 39 | ++ |
| 43 | + |
| 47 | ++ |
| 48 | + |
| 55 | + |
| 56 | + |
| 57 | +++ |
| 60 | +++ |
| 61 | + |
| 62 | + |
| 63 | + |
| 64 | +++ |
| 67 | + |
| 68 | + |
| 71 | ++ |
| 72 | + |
| 73 | + |
| 80 | + |
| 81 | +++ |
| 82 | + |
| 83 | + |
| 84 | ++ |
| 85 | + |
| 86 | + |
| 89 | + |
| 91 | + |
| 92 | + |
| 93 | +++ |
| 94 | ++ |
| 95 | + |
| 98 | ++ |
| 101 | +++ |
| 102 | + |
| 103 | +++ |
| 105 | +++ |
| 107 | +++ |
| 110 | +++ |
| 111 | +++ |
| 114 | +++ |
| 115 | + |
| 119 | + |
| 120 | +++ |
| 121 | + |
| 123 | + |
| 126 | ++++ |
| 127 | ++++ |
| 129 | + |
| 130 | +++ |
| 131 | + |
| 132 | +++ |
| 133 | ++ |
| 134 | +++ |
| 135 | + |
| 136 | ++ |
| 137 | + |
| 139 | +++ |
| 140 | + |
| 141 | + |
| 142 | + |
| 143 | ++ |
| 144 | ++ |
| 147 | + |
| 148 | ++ |
| 149 | +++ |
| 150 | +++ |
| 151 | ++++ |
| 153 | +++ |
| 154 | + |
| 155 | +++ |
| 156 | + |
| 157 | + |
| 158 | +++ |
| 159 | ++ |
| 160 | + |
| 161 | + |
| 162 | +++ |
| 163 | ++ |
| 164 | +++ |
| 165 | N/A |
| 166 | + |
| 167 | + |
| 168 | +++ |
| 172 | +++ |
| 176 | ++++ |
| 177 | + |
| 178 | + |
| 179 | +++ |
| 182 | ++ |
| 186 | +++ |
| 189 | + |
| 190 | ++++ |
| 191 | +++ |
| 193 | +++ |
| 194 | + |
| 195 | +++ |
| 196 | +++ |
| 197 | ++++ |
| 198 | +++ |
| 199 | ++++ |
| 200 | +++ |
| 201 | +++ |
| 202 | +++ |
| 203 | ++++ |
| 204 | ++++ |
| 205 | +++ |
| 206 | +++ |
| 207 | +++ |
| 208 | +++ |
| 209 | +++ |
| 210 | ++++ |
| 211 | ++ |
| 212 | + |
| 213 | +++ |
| 214 | ++++ |
| 215 | +++ |
| 216 | +++ |
| 217 | + |
| 218 | + |
| 219 | + |
| 220 | +++ |
| 221 | + |
| 222 | +++ |
| 223 | +++ |
| 224 | +++ |
| 225 | ++++ |
| 226 | +++ |
| 227 | ++ |
| 228 | ++++ |
| 229 | +++ |
| 230 | ++++ |
| 231 | +++ |
| 232 | ++++ |
| 233 | ++++ |
| 234 | ++++ |
| 235 | +++ |
| 236 | ++++ |
| 237 | +++ |
| 238 | +++ |
| 239 | +++ |
| 240 | ++ |
| 241 | +++ |
| 242 | ++ |
| 243 | +++ |
| 244 | ++++ |
| 245 | +++ |
| 246 | ++++ |
| 247 | ++++ |
| 248 | ++++ |
| 249 | ++++ |
| 250 | ++ |
| 251 | +++ |
| 252 | + |
| 253 | ++++ |
| 254 | + |
| 255 | +++ |
| 256 | ++++ |
| 257 | ++++ |
| 258 | +++ |
| 259 | ++ |
| 260 | +++ |
| 261 | ++++ |
| 262 | +++ |
| 263 | + |
| 264 | +++ |
| 265 | + |
| 266 | + |
| 267 | + |
| 269 | +++ |
| 270 | + |
| 271 | ++ |
| 272 | ++ |
| 274 | +++ |
| 276 | + |
| 277 | +++ |
| 280 | + |
| 283 | + |
| 284 | +++ |
| 287 | ++++ |
| 288 | ++++ |
| 289 | ++++ |
| 290 | ++++ |
| 291 | + |
| 292 | ++++ |
| 293 | + |
| 294 | ++++ |
| 295 | +++ |
| 299 | + |
| 300 | + |
| 301 | ++++ |
| 302 | + |
| 303 | N/A |
| 304 | ++++ |
| 305 | N/A |
| 306 | N/A |
| 307 | +++ |
| 308 | + |
| 309 | N/A |
| 310 | N/A |
| 311 | +++ |
| 312 | + |
| 313 | ++ |
| 314 | N/A |
| 315 | N/A |
| 316 | + |
| 317 | ++++ |
| 318 | N/A |
| 319 | ++++ |
| 320 | ++ |
| 321 | N/A |
| 322 | + |
| 323 | ++++ |
| 324 | ++++ |
| 325 | +++ |
| 326 | +++ |
| 327 | ++++ |
| 328 | + |
| 329 | + |
| 330 | + |
| 331 | + |
| 332 | + |
| 333 | ++ |
| 334 | +++ |
| 335 | N/A |
| 336 | N/A |
| 337 | N/A |
| 338 | N/A |
| 339 | N/A |
| 340 | N/A |
| 341 | + |
| 342 | + |
| 343 | +++ |
| 344 | ++++ |
| 345 | N/A |
| 346 | +++ |
| 347 | +++ |
| 348 | + |
| 349 | +++ |
| 350 | + |
| 351 | +++ |
| 352 | ++++ |
| 353 | ++++ |
| 354 | ++++ |
| 355 | +++ |
| 356 | ++++ |
| 357 | ++++ |
| 358 | +++ |
| 359 | N/A |
| 360 | N/A |
| 362 | + |
| 363 | ++++ |
| 364 | +++ |
| 365 | +++ |
| 366 | N/A |
| 367 | N/A |
| 368 | N/A |
| 369 | +++ |
| 370 | ++++ |
| 371 | N/A |
| 372 | ++ |
| 373 | ++ |
| 374 | N/A |
| 375 | +++ |
| 376 | +++ |
| 377 | + |
| 378 | ++ |
| 379 | +++ |
| 380 | +++ |
| 381 | + |
| 382 | N/A |
| 383 | ++ |
| 384 | + |
| 385 | ++++ |
| 386 | N/A |
| 387 | +++ |
| 388 | ++++ |
| 389 | ++++ |
| 390 | ++++ |
| 391 | +++ |
| 392 | +++ |
| 393 | ++++ |
| 394 | +++ |
| 395 | +++ |
| 396 | ++++ |
| 397 | ++++ |
| 398 | ++++ |
| 399 | N/A |
| 400 | + |
| 401 | N/A |
| 402 | +++ |
| 403 | N/A |
| 404 | +++ |
| 405 | ++ |
| 406 | ++++ |
| 407 | ++ |
| 408 | N/A |
| 409 | ++ |
| 410 | ++++ |
| 411 | ++++ |
| 412 | N/A |
| 413 | +++ |
| 414 | +++ |
| 415 | ++++ |
| 416 | ++++ |
| 417 | +++ |
| 418 | ++++ |
| 419 | + |
| 420 | + |
| 421 | ++ |
| 422 | + |
| 423 | ++++ |
| 424 | +++ |
| 425 | +++ |
| 426 | +++ |
| 427 | N/A |
| 428 | N/A |
| 429 | ++++ |
| 430 | ++++ |
| 431 | +++ |
| 432 | + |
| 433 | +++ |
| 434 | + |
| 435 | +++ |
| 436 | +++ |
| 437 | +++ |
| 438 | +++ |
| 439 | ++ |
| 440 | +++ |
| 441 | +++ |
| 442 | N/A |
| 443 | +++ |
| 444 | +++ |
| 445 | +++ |
| 446 | ++ |
| 447 | +++ |
| 448 | +++ |
| 449 | + |
| 450 | +++ |
| 451 | +++ |
| 452 | ++++ |
| 453 | ++ |
| 454 | +++ |
| 455 | ++++ |
| 456 | N/A |
| 457 | N/A |
| 458 | ++ |
| 459 | + |
| 460 | + |
| 461 | + |
| 462 | + |
| 463 | +++ |
| 464 | + |

N/A not active
+ >1001 nM
++ 501-1000 nM
+++ 101-500 nM
++++ 0.01-100 nM

### 1.3.1 JAK3 Ki Determination Assay

For the determination of Ki, different amounts of compound are mixed with the enzyme and the enzymatic reaction is followed as a function of ATP concentration. The Ki is determined by means of double reciprocal plotting of Km vs compound concentration (Lineweaver-Burk plot). JAK3 (Carna Biosciences, 09CBS-0625B) is used at a final concentration of 10 ng/mL. The substrate is Poly(Glu,Tyr)sodium salt (4:1), MW 20 000-50 000 (Sigma, P0275) The reaction is performed in 25 mM Tris pH 7.5, 0.01% Triton X-100, 0.5 mM EGTA, 2.5 mM DTT, 0.5 mM Na₃VO₄, 5 mM b-glycerolphosphate, 10 mM MgCl₂ with varying concentrations of ATP and compound and stopped by addition of 150 mM phosphoric acid. Measurement of incorporated phosphate into the substrate polyGT is done by loading the samples on a filter plate (using a harvester, Perkin Elmer) and subsequent washing. Incorporated ³³P in polyGT is measured in a Topcount scintillation counter after addition of scintillation liquid to the filter plates (Perkin Elmer).

### 1.4 TYK2 Inhibition Assay

Recombinant human TYK2 catalytic domain (amino acids 871-1187; catalog number 08-147) was purchased from Carna biosciences. 5 ng of TYK2 was incubated with 12.5 μg polyGT substrate (Sigma catalog number P0275) in kinase reaction buffer (25 mM Hepes pH 7.2, 50 mM NaCl, 0.5 mM EDTA, 1 mM DTT, 5 mM MnCl₂, 10 mM MgCl₂, 0.1% Brij-35, 0.1 μM non-radioactive ATP, 0.125 μCi ³³P-gamma-ATP (GE Healthcare, catalog number AH9968) final concentrations) with or without 5 μL containing test compound or vehicle (DMSO, 1% final concentration), in a total volume of 25 μL, in a polypropylene 96-well plate (Greiner, V-bottom). After 90 min at 30° C., reactions were stopped by adding 25 μL/well of 150 mM phosphoric acid. All of the terminated kinase reaction was transferred to prewashed (75 mM phosphoric acid) 96 well filter plates (Perkin Elmer catalog number 6005177) using a cell harvester (Perkin Elmer). Plates were washed 6 times with 300 μL per well of a 75 mM phosphoric acid solution and the bottom of the plates was sealed. 40 μL/well of Microscint-20 was added, the top of the plates was sealed and readout was performed using the Topcount (Perkin Elmer). Kinase activity was calculated by subtracting counts per min (cpm) obtained in the presence of a positive control inhibitor (10 μM staurosporine) from cpm obtained in the presence of vehicle. The ability of a test compound to inhibit this activity was determined as:

Percentage inhibition=((cpm determined for sample with test compound present−cpm determined for sample with positive control inhibitor)divided by(cpm determined in the presence of vehicle−cpm determined for sample with positive control inhibitor))*100.

Dose dilution series were prepared for the compounds enabling the testing of dose-response effects in the TYK2 assay and the calculation of the IC50 for each compound. Each compound was routinely tested at concentration of 20 μM followed by a 1/3 serial dilution, 8 points (20 μM—6.67 μM—2.22 μM—740 nM—247 nM—82 nM—27 nM—9 nM) in a final concentration of 1% DMSO. When potency of compound series increased, more dilutions were prepared and/or the top concentration was lowered (e.g. 5 μM, 1 μM).

The following compounds have been tested for their activity against TYK2; and the IC50 values, as determined using the assays described herein, are given below in Table VI.

TABLE VI
TYK2 IC50 Values of Compounds

| Cpd # | TYK2 |
|---|---|
| 1 | § |
| 2 | § |
| 4 | §§§ |
| 5 | §§ |
| 16 | §§§ |
| 17 | §§§ |
| 26 | §§§ |
| 27 | § |
| 28 | §§§ |
| 29 | §§ |
| 30 | § |
| 31 | §§ |
| 32 | § |
| 33 | § |
| 34 | §§§§ |
| 35 | §§§ |
| 38 | §§§ |
| 39 | §§ |
| 43 | § |
| 47 | §§§ |
| 48 | §§ |
| 55 | § |
| 56 | § |
| 57 | §§ |
| 60 | §§ |
| 61 | § |
| 62 | § |
| 63 | § |
| 64 | §§§ |
| 67 | §§§ |
| 68 | §§ |
| 71 | §§§§ |
| 72 | §§ |
| 73 | §§§ |
| 80 | §§§ |
| 81 | §§§§ |
| 82 | §§§ |
| 83 | §§§ |
| 84 | §§§ |
| 85 | §§§ |
| 86 | §§ |
| 89 | N/A |
| 91 | § |
| 92 | § |
| 93 | §§§§ |
| 94 | §§§§ |
| 95 | §§§§ |
| 98 | §§ |
| 101 | §§§§ |
| 102 | §§§ |
| 103 | §§§§ |
| 105 | §§§§ |
| 107 | §§§§ |
| 110 | §§§§ |
| 111 | §§§ |
| 114 | §§§§ |
| 115 | §§§§ |
| 119 | § |
| 120 | §§§ |
| 121 | §§§§ |
| 123 | N/A |
| 126 | §§§§ |
| 127 | §§§§ |
| 129 | §§§ |
| 130 | §§§§ |
| 131 | §§§ |
| 132 | §§§§ |
| 133 | §§§ |
| 134 | §§§§ |
| 135 | § |
| 136 | §§§§ |
| 137 | §§§ |
| 139 | §§§§ |
| 140 | §§§ |
| 141 | § |
| 142 | §§§ |
| 143 | §§ |
| 144 | §§§§ |
| 147 | §§§§ |
| 148 | §§§§ |
| 149 | §§§§ |
| 150 | §§§§ |
| 151 | §§§§ |
| 153 | §§§§ |
| 154 | §§§ |
| 155 | §§§§ |
| 156 | §§§§ |
| 157 | §§§§ |
| 158 | §§§§ |
| 159 | §§§ |
| 160 | § |
| 161 | § |
| 162 | §§§§ |
| 163 | §§ |
| 164 | §§§§ |
| 165 | § |
| 166 | § |
| 167 | §§ |
| 168 | §§§§ |
| 172 | §§§§ |
| 176 | §§§§ |
| 177 | §§§§ |
| 178 | §§§ |
| 179 | §§§§ |
| 182 | §§§ |
| 186 | §§§§ |
| 189 | §§§ |
| 190 | §§§§ |
| 191 | §§§ |
| 193 | §§§§ |
| 194 | §§ |
| 195 | §§§§ |
| 196 | §§§§ |
| 197 | §§§§ |
| 198 | §§§§ |
| 199 | §§§§ |
| 200 | §§§§ |
| 201 | §§§§ |
| 202 | §§§§ |
| 203 | §§§§ |
| 204 | §§§§ |
| 205 | §§§§ |
| 206 | §§§§ |
| 207 | §§§§ |
| 208 | §§§§ |
| 209 | §§§ |
| 210 | §§§§ |
| 211 | §§§§ |
| 212 | §§§ |
| 213 | §§§§ |
| 214 | §§§§ |
| 215 | §§§§ |
| 216 | §§§§ |
| 217 | §§§ |
| 218 | §§ |
| 219 | §§§§ |
| 220 | §§§§ |
| 221 | §§§ |
| 222 | §§§§ |
| 223 | §§§§ |
| 224 | §§§§ |
| 225 | §§§§ |
| 226 | §§§§ |
| 227 | §§§ |
| 228 | §§§§ |
| 229 | §§§§ |
| 230 | §§§§ |
| 231 | §§§§ |
| 232 | §§§§ |
| 233 | §§§§ |
| 234 | §§§§ |
| 235 | §§§§ |
| 236 | §§§§ |
| 237 | §§§§ |
| 238 | §§§§ |
| 239 | §§§ |
| 240 | §§§§ |
| 241 | §§§ |
| 242 | §§§ |
| 243 | §§§ |
| 244 | §§§§ |
| 245 | §§§ |
| 246 | §§§§ |
| 247 | §§§§ |
| 248 | §§§§ |
| 249 | §§§§ |
| 250 | §§§§ |
| 251 | §§§§ |
| 252 | § |
| 253 | §§§§ |
| 254 | § |
| 255 | §§§ |
| 256 | §§§ |
| 257 | §§§§ |
| 258 | §§§ |
| 259 | §§§ |
| 260 | §§§ |
| 261 | §§§§ |
| 262 | §§§ |
| 263 | § |
| 264 | §§§§ |
| 265 | § |
| 266 | § |
| 267 | § |
| 269 | §§§§ |
| 270 | § |
| 271 | §§§§ |
| 272 | §§§§ |
| 274 | §§§§ |
| 276 | § |
| 277 | §§ |
| 280 | §§§ |
| 283 | §§§ |
| 284 | §§§§ |
| 287 | §§§§ |
| 288 | §§§§ |
| 289 | §§§§ |
| 290 | §§§§ |
| 291 | §§§ |
| 292 | §§§ |
| 293 | § |
| 294 | §§§§ |
| 295 | §§§§ |
| 299 | §§§ |
| 300 | §§ |
| 301 | §§§§ |
| 302 | §§§ |
| 303 | N/A |
| 304 | §§§§ |
| 305 | N/A |
| 306 | N/A |
| 307 | §§§ |
| 308 | § |
| 309 | N/A |
| 310 | N/A |
| 311 | §§§§ |
| 312 | § |
| 313 | §§§ |
| 314 | N/A |
| 315 | N/A |
| 316 | § |
| 317 | §§§§ |
| 318 | N/A |
| 319 | §§§§ |
| 320 | §§§ |
| 321 | N/A |
| 322 | §§§ |
| 323 | §§§§ |
| 324 | §§§§ |
| 325 | §§§ |
| 326 | §§§ |
| 327 | §§§§ |
| 328 | §§ |
| 329 | § |
| 330 | § |
| 331 | §§ |
| 332 | § |
| 333 | §§ |
| 334 | §§ |
| 335 | N/A |
| 336 | N/A |
| 337 | N/A |
| 338 | N/A |
| 339 | N/A |
| 340 | N/A |
| 341 | § |
| 342 | § |
| 343 | §§ |
| 344 | §§§ |
| 345 | N/A |
| 346 | §§§§ |
| 347 | §§§ |
| 348 | § |
| 349 | §§§§ |
| 350 | §§§ |
| 351 | §§§ |
| 352 | §§§§ |
| 353 | §§§§ |
| 354 | §§§§ |
| 355 | §§§ |
| 356 | §§§§ |
| 357 | §§§§ |
| 358 | §§§ |
| 359 | N/A |
| 360 | N/A |
| 362 | § |
| 363 | §§§ |
| 364 | § |
| 365 | §§ |
| 366 | N/A |
| 367 | N/A |
| 368 | N/A |
| 369 | §§§ |
| 370 | §§§ |
| 371 | N/A |
| 372 | §§§ |
| 373 | § |
| 374 | N/A |
| 375 | §§§ |
| 376 | §§§ |
| 377 | § |
| 378 | §§§ |
| 379 | §§§ |
| 380 | §§§ |
| 381 | §§ |
| 382 | N/A |
| 383 | §§ |
| 384 | §§§ |
| 385 | §§§§ |
| 386 | N/A |
| 387 | §§§ |
| 388 | §§§§ |
| 389 | §§§§ |
| 390 | §§§§ |
| 391 | §§§§ |
| 392 | §§§§ |
| 393 | §§§§ |
| 394 | §§§§ |
| 395 | §§§§ |
| 396 | §§§§ |
| 397 | §§§§ |
| 398 | §§§ |
| 399 | N/A |
| 400 | §§ |
| 401 | N/A |
| 402 | §§§§ |
| 403 | N/A |
| 404 | §§§ |
| 405 | §§§§ |
| 406 | §§§§ |
| 407 | §§§ |
| 408 | N/A |
| 409 | § |
| 410 | §§§§ |
| 411 | §§§§ |
| 412 | N/A |
| 413 | §§§§ |
| 414 | §§§§ |
| 415 | §§§§ |
| 416 | §§§ |
| 417 | §§§§ |
| 418 | §§§§ |
| 419 | § |
| 420 | §§§ |
| 421 | §§§§ |
| 422 | §§ |
| 423 | §§§§ |
| 424 | §§§ |
| 425 | §§§ |
| 426 | §§§ |
| 427 | N/A |
| 428 | N/A |
| 429 | §§§ |
| 430 | §§§§ |
| 431 | § |
| 432 | §§§ |
| 433 | §§§ |
| 434 | §§§ |
| 435 | §§§§ |
| 436 | §§§§ |
| 437 | §§§§ |
| 438 | §§§§ |
| 439 | §§§§ |
| 440 | §§§§ |
| 441 | §§§§ |
| 442 | N/A |
| 443 | §§§§ |
| 444 | §§§§ |
| 445 | §§§§ |
| 446 | §§§§ |
| 447 | §§§§ |
| 448 | §§§§ |
| 449 | §§§§ |
| 450 | §§§§ |
| 451 | §§§§ |
| 452 | §§§§ |
| 453 | §§§§ |
| 454 | §§§§ |
| 455 | §§§§ |
| 456 | N/A |
| 457 | N/A |
| 458 | §§ |
| 459 | § |
| 460 | § |
| 461 | § |
| 462 | § |
| 463 | §§§§ |
| 464 | § |

N/A not active
§ >1001 nM
§§ 501-1000 nM
§§§ 101-500 nM
§§§§ 0.01-100 nM

### 1.4.1 TYK2 Kd Determination Assay

TYK2 (Cama Biosciences, 09CBS-0983D) is used at a final concentration of 5 nM. The binding experiment is performed in 50 mM Hepes pH 7.5, 0.01% Brij-35, 10 mM MgCl₂, 1 mM EGTA using 50 nM kinase tracer 236 (Invitrogen, PV5592) and 2 nM Eu-anti-GST (Invitrogen, PV5594) with varying compound concentrations. Detection of tracer is performed according to the manufacturers' procedure.

### Example 2

### Cellular Assays

### Example 2.1

### JAK-STAT Signalling Assay

HeLa cells were maintained in Dulbecco's Modified Eagle's Medium (DMEM) containing 10% heat inactivated fetal calf serum, 100 U/mL penicillin and 100 μg/mL streptomycin. HeLa cells were used at 70% confluence for transfection. 20,000 cells in 87 μL cell culture medium were transiently transfected with 40 ng pSTAT1(2)-luciferase reporter (Panomics), 8 ng of LacZ reporter as internal control reporter and 52 ng of pBSK using 0.32 μL Jet-PEI (Polyplus) as transfection reagent per well in 96-well plate format. After overnight incubation at 37° C., 5% CO₂, transfection medium was removed. 81 μL of DMEM+1.5% heat inactivated fetal calf serum was added. 9 μL compound at 10× concentration was added for 60 min and then 10 μL of human OSM (Peprotech) at 33 ng/mL final concentration.

All compounds were tested in duplicate starting from 20 μM followed by a 1/3 serial dilution, 8 doses in total (20 μM—6.6 μM—2.2 μM—740 nM—250 nM—82 nM—27 nM—9 nM) in a final concentration of 0.2% DMSO.

After overnight incubation at 37° C., 5% CO₂ cells were lysed by adding 100 μL lysis buffer/well (PBS, 0.9 mM CaCl₂, 0.5 mM MgCl₂, 10% Trehalose, 0.05% Tergitol NP9, 0.3% BSA).

40 μL of cell lysate was used to read β-galactosidase activity by adding 180 μL β-Gal solution (30 μL ONPG 4 mg/mL+150 μL β-Galactosidase buffer (0.06 M Na₂HPO₄, 0.04 M NaH₂PO₄, 1 mM MgCl₂)) for 20 min. The reaction was stopped by addition of 50 μL Na₂CO₃ 1 M. Absorbance was read at 405 nm.

Luciferase activity was measured using 40 μL cell lysate plus 40 μL of Steadylite as described by the manufacturer (Perkin Elmer), on the Envision (Perkin Elmer).

Omitting OSM was used as a positive control (100% inhibition). As negative control 0.5% DMSO (0% inhibition) was used. The positive and negative controls were used to calculate z′ and ‘percent inhibition’ (PIN) values.

Percentage inhibition=((fluorescence determined in the presence of vehicle−fluorescence determined for sample with test compound present)divided by(fluorescence determined in the presence of vehicle−fluorescence determined for sample without trigger))*100.

PIN values were plotted for compounds tested in dose-response and EC₅₀ values were derived.

The following compounds have been tested for their activity in the JAK-STAT assay; and the IC₅₀ values, as determined using the assays described herein, are given below in Table VII.

TABLE VII
JAK-STAT IC₅₀ of selected compounds of the invention

| | JAK- |
|---|---|
| Cpd # | STAT |
| 16 | & |
| 17 | & |
| 26 | & |
| 27 | & |
| 28 | & |
| 29 | & |
| 30 | & |
| 31 | & |
| 32 | & |
| 33 | & |
| 34 | & |
| 35 | & |
| 38 | & |
| 39 | & |
| 43 | & |
| 47 | & |
| 48 | & |
| 55 | N/A |
| 56 | & |
| 57 | & |
| 60 | & |
| 61 | & |
| 62 | & |
| 63 | & |
| 64 | & |
| 67 | N/A |
| 68 | N/A |
| 71 | & |
| 72 | & |
| 73 | & |
| 80 | & |
| 81 | &&& |
| 82 | & |
| 83 | & |
| 84 | && |
| 85 | & |
| 86 | & |
| 91 | & |
| 92 | N/A |
| 93 | &&& |
| 94 | &&& |
| 95 | && |
| 98 | & |
| 101 | &&& |
| 102 | & |
| 103 | & |
| 105 | & |
| 107 | && |
| 110 | & |
| 111 | && |
| 114 | &&& |
| 115 | &&& |
| 119 | & |
| 120 | & |
| 121 | && |
| 123 | & |
| 126 | &&&& |
| 127 | &&& |
| 129 | & |
| 131 | && |
| 132 | && |
| 133 | &&& |
| 134 | & |
| 135 | & |
| 136 | && |
| 137 | && |
| 139 | && |
| 140 | & |
| 141 | & |
| 142 | && |
| 143 | & |
| 144 | & |
| 147 | && |
| 148 | && |
| 149 | & |
| 150 | &&& |
| 151 | &&& |
| 153 | &&& |
| 154 | & |
| 155 | && |
| 156 | && |
| 157 | & |
| 158 | &&& |
| 159 | & |
| 160 | & |
| 161 | N/A |
| 162 | &&&& |
| 163 | && |
| 164 | && |
| 165 | N/A |
| 166 | N/A |
| 167 | & |
| 168 | &&& |
| 172 | &&& |
| 176 | &&& |
| 177 | && |
| 178 | && |
| 179 | N/A |
| 182 | & |
| 186 | &&& |
| 189 | & |
| 190 | &&& |
| 191 | && |
| 193 | & |
| 194 | & |
| 195 | && |
| 196 | && |
| 197 | &&& |
| 198 | && |
| 199 | &&& |
| 200 | && |
| 201 | &&& |
| 202 | &&& |
| 203 | & |
| 204 | &&& |
| 205 | &&& |
| 206 | & |
| 207 | && |
| 208 | &&& |
| 209 | && |
| 210 | &&& |
| 211 | && |
| 212 | & |
| 213 | &&& |
| 214 | &&& |
| 215 | & |
| 216 | & |
| 217 | & |
| 218 | & |
| 219 | &&& |
| 220 | && |
| 221 | & |
| 222 | &&& |
| 223 | &&& |
| 224 | && |
| 225 | && |
| 226 | & |
| 227 | && |
| 228 | &&& |
| 229 | &&& |
| 230 | &&& |
| 231 | &&& |
| 232 | & |
| 233 | &&& |
| 234 | &&& |
| 235 | && |
| 236 | &&& |
| 237 | &&& |
| 238 | &&& |
| 239 | &&& |
| 240 | && |
| 241 | &&& |
| 242 | & |
| 243 | &&& |
| 244 | &&& |
| 245 | && |
| 246 | &&& |
| 247 | &&& |
| 248 | &&& |
| 249 | &&& |
| 250 | &&& |
| 251 | &&& |
| 252 | & |
| 253 | &&& |
| 254 | & |
| 255 | & |
| 256 | &&& |
| 257 | &&& |
| 258 | & |
| 259 | && |
| 261 | &&&& |
| 262 | &&& |
| 263 | & |
| 264 | &&&& |
| 265 | & |
| 266 | & |
| 267 | & |
| 269 | && |
| 270 | & |
| 271 | &&& |
| 272 | && |
| 274 | &&& |
| 276 | & |
| 277 | & |
| 280 | & |
| 283 | & |
| 284 | && |
| 287 | &&& |
| 295 | &&& |
| 302 | & |
| 303 | & |
| 304 | &&& |
| 311 | &&& |
| 312 | & |
| 313 | & |
| 314 | & |
| 315 | & |
| 316 | & |
| 319 | && |
| 320 | & |
| 322 | & |
| 323 | &&& |
| 324 | &&& |
| 325 | &&& |
| 326 | && |
| 327 | &&& |
| 328 | & |
| 329 | &&& |
| 330 | & |
| 331 | & |
| 332 | & |
| 333 | & |
| 334 | & |
| 343 | && |
| 463 | & |

& >1001 nM
&& 501-1000 nM
&&& 1-500 nM

### Example 2.2

### OSM/IL-1/3 Signaling Assay

OSM and IL-1β are shown to synergistically upregulate MMP13 levels in the human chondrosarcoma cell line SW1353. The cells are seeded in 96 well plates at 15,000 cells/well in a volume of 120 μL DMEM (Invitrogen) containing 10% (v/v) FBS and 1% penicillin/streptomycin (InVitrogen) incubated at 37° C. 5% CO₂. Cells are preincubated with 15 μL of compound in M199 medium with 2% DMSO 1 hr before triggering with 15 μL OSM and IL-113 to reach 25 ng/mL OSM and 1 ng/mL IL-113, and MMP13 levels are measured in conditioned medium 48 h after triggering. MMP13 activity is measured using an antibody capture activity assay. For this purpose, 384 well plates (NUNC, 460518, MaxiSorb black) are coated with 35 μL of a 1.5 μg/mL anti-human MMP13 antibody (R&D Systems, MAB511) solution for 24 h at 4° C. After washing the wells 2 times with PBS+0.05% Tween, the remaining binding sites are blocked with 100 μL 5% non-fat dry milk (Santa Cruz, sc-2325, Blotto) in PBS for 24 hr at 4° C. Next, the wells are washed twice with PBS+0.05% Tween and 35 μL of 1/10 dilution of culture supernatant containing MMP13 in 100-fold diluted blocking buffer is added and incubated for 4 hr at room temperature. Next the wells are washed twice with PBS+0.05% Tween followed by MMP13 activation by addition of 35 μL of a 1.5 mM 4-Aminophenylmercuric acetate (APMA) (Sigma, A9563) solution and incubation at 37° C. for 1 hr. The wells are washed again with PBS+0.05% Tween and 35 μL MMP13 substrate (Biomol, P-126, OmniMMP fluorogenic substrate) is added. After incubation for 24 h at 37° C. fluorescence of the converted substrate is measured in a Perkin Elmer Wallac EnVision 2102 Multilabel Reader (wavelength excitation: 320 nm, wavelength emission: 405 nm).

Percentage inhibition=((fluorescence determined in the presence of vehicle−fluorescence determined for sample with test compound present)divided by(fluorescence determined in the presence of vehicle−fluorescence determined for sample without trigger))100.

### Example 2.3

### PBL Proliferation Assay

Human peripheral blood lymphocytes (PBL) are stimulated with IL-2 and proliferation is measured using a BrdU incorporation assay. The PBL are first stimulated for 72 h with PHA to induce IL-2 receptor, then they are fasted for 24 h to stop cell proliferation followed by IL-2 stimulation for another 72 h (including 24 hr BrdU labeling). Cells are preincubated with test compounds I hr before IL-2 addition. Cells are cultured in RPMI 1640 containing 10% (v/v) FBS.

### Example 2.4

### Whole Blood Assay (WBA)

### 2.4.1 IFNα Stimulation Protocol

To predict the potency of the test compounds to inhibit JAK1 or JAK2-dependent signaling pathways in vivo, a physiologically relevant in vitro model was developed using human whole blood. In the WBA assay, blood, drawn from human volunteers who gave informed consent, is treated ex vivo with compound (1 h) and subsequently stimulated either for 30 min with interferon α (IFNα, JAK1 dependent pathway) or for 2 h with granulocyte macrophage-colony stimulating factor (GM-CSF, JAK2 dependent pathway).

### 2.4.1.1 Phospho-STAT1 Assay

For IFNα stimulation, increase in phosphorylation of Signal Transducers and Activators of Transcription 1 (pSTAT1) by IFNα in white blood cell extracts is measured using a pSTAT1 ELISA assay. Phosphorylation of Signal Transducer and Activator of Transcription 1 (STAT1) after interferon alpha (IFNα) triggering is a JAK1-mediated event. The Phospho-STAT1 Assay, which is used to measure Phospho-STAT1 levels in cellular extracts, is developed to assess the ability of a compound to inhibit JAK1-dependent signaling pathways.

Whole human blood, drawn from human volunteers who gave informed consent, is ex vivo treated with compound (1 h) and subsequently stimulated for 30 min with IFNα. The increase in phosphorylation of STAT1 by INFα in white blood cell extracts was measured using a phospho-STAT1 ELISA.

The ACK lysis buffer consisted of 0.15 M NH₄Cl, 10 mM KHCO₃, 0.1 mM EDTA. The pH of the buffer was 7.3.

A 10× cell lysis buffer concentrate (part of the PathScan Phospho-STAT1 (Tyr701) sandwich ELISA kit from Cell Signaling) is diluted 10-fold in H₂O. Proteinase inhibitors were added to the buffer before use.

20 μg IFNα is dissolved in 40 μL H₂O to obtain a 500 μg/mL stock solution. The stock solution was stored at −20° C.

A 3-fold dilution series of the compound is prepared in DMSO (highest concentration: 10 mM). Subsequently, the compound is further diluted in medium (dilution factor dependent on desired final compound concentration).

2.4.1.1.1 Incubation of Blood with Compound and Stimulation with IFNα

Human blood is collected in heparinized tubes. The blood is divided in aliquots of 392 μL. Afterwards, 4 μL of compound dilution is added to each aliquot and the blood samples are incubated for 1 h at 37° C. The IFNα stock solution is diluted 1000-fold in RPMI medium to obtain a 500 ng/mL working solution. 4 μL of the 500 ng/mL work solution is added to the blood samples (final concentration IFNα: 5 ng/mL). The samples are incubated at 37° C. for 30 min.

### 2.4.1.1.2 Preparation of Cell Extracts

At the end of the stimulation period, 7.6 mL ACK buffer is added to the blood samples to lyse the red blood cells. The samples are mixed by inverting the tubes five times and the reaction is incubated on ice for 5 min. The lysis of the RBC should be evident during this incubation. The cells are pelleted by centrifugation at 300 g, 4° C. for 7 min and the supernatant is removed. 10 mL 1×PBS is added to each tube and the cell pellet is resuspended. The samples are centrifuged again for 7 min at 300 g, 4° C. The supernatant is removed and the pellet resuspended in 500 μL of 1×PBS. Then, the cell suspension is transferred to a clean 1.5 mL microcentrifuge tube. The cells are pelleted by centrifugation at 700 g for 5 min at 4° C. The supernatant is removed and the pellet was dissolved in 150 μL cell lysis buffer. The samples are incubated on ice for 15 min. After that, the samples are stored at −80° C. until further processing.

### 2.4.1.1.3 Measurement of STAT1 Phosphorylation by ELISA

The Pathscan Phospho-STAT1 (Tyr701) Sandwich ELISA kit from Cell Signaling (Cat. no: #7234) is used to determine Phospho-STAT1 levels.

The cellular extracts are thawed on ice. The tubes are centrifuged for 5 min at 16,000 g, 4° C. and the cleared lysates are harvested. Meanwhile, the microwell strips from the kit are equilibrated to room temperature and wash buffer is prepared by diluting 20× wash buffer in H₂0, Samples are diluted 2-fold in sample diluent and 100 μL is added to the microwell strips. The strips are incubated overnight at 4° C.

The following day, the wells are washed 3 times with wash buffer. 100 μL of the detection antibody is added to the wells. The strips are incubated at 37° C. for 1 h. Then, the wells are washed 3 times with wash buffer again. 100 μL HRP-linked secondary antibody is added to each well and the samples are incubated at 37° C. After 30 min, the wells are washed 3 times again and 100 μL TMB substrate is added to all wells. When samples turned blue, 100 μL STOP solution is added to stop the reaction. Absorbance is measured at 450 nm.

### 2.4.1.2 Data Analysis

Inhibition of phosphoSTAT1 induction by IFNα in cell extracts is plotted against the compound concentration and IC₅₀ values are derived using Graphpad software. Data were retained if R² (coefficient of determination used in statistical models to measure the proportion of variability of the model and its predictive capacity. R² ranges from 0 (no correlation of the data: no predictive value) to 1 (full correlation: great predictive value) is larger than 0.8 and the hill slope is smaller than 3.

### 2.4.1.3 IL-8 ELISA

For GM-CSF stimulation, increase in interleukin-8 (IL-8) levels in plasma is measured using an IL-8 ELISA assay. Granulocyte macrophagecolony stimulating factor (GM-CSF)— induced interleukin 8 (IL-8) expression is a JAK2-mediated event. The IL-8 ELISA, which can be used to measure IL-8 levels in plasma samples, has been developed to assess the ability of a compound to inhibit JAK2-dependent signaling pathways.

Whole human blood, drawn from human volunteers who gave informed consent, is ex vivo treated with compound (1 h) and subsequently stimulated for 2 h with GM-CSF. The increase in IL-8 levels in plasma is measured using an IL-8 ELISA assay.

10 μg GM-CSF is dissolved in 100 μL H₂O to obtain a 100 μg/mL stock solution. The stock solution is stored at −20° C.

A 3-fold dilution series of the test compound is prepared in DMSO (highest concentration: 10 mM). Subsequently, the compound is further diluted in medium (dilution factor dependent on desired final compound concentration).

2.4.1.3.1 Incubation of Blood with Compound and Stimulation with GM-CSF

Human blood is collected in heparinized tubes. The blood is divided in aliquots of 245 μL. Afterwards, 2.5 μL test compound dilution is added to each aliquot and the blood samples are incubated for 1 h at 37° C. The GM-CSF stock solution is diluted 100-fold in RPMI medium to obtain a 1 μg/mL work solution. 2.5 μL of the 1 μg/mL work solution is added to the blood samples (final concentration GM-CSF: 10 ng/mL). The samples are incubated at 37° C. for 2 h.

### 2.4.1.3.2 Preparation of Plasma Samples

The samples are centrifuged for 15 min at 1,000 g, 4° C. 100 μL of the plasma is harvested and stored at −80° C. until further use.

### 2.4.1.3.3 Measurement of IL-8 Levels by ELISA

The Human IL-8 Chemiluminescent Immunoassay kit from R&D Systems (Cat. no: Q8000B) is used to determine IL-8 levels.

Wash buffer is prepared by diluting 10× wash buffer in H₂O. Working glo reagent is prepared by adding 1 part Glo Reagent 1 to 2 parts Glo Reagent B 15 min to 4 h before use. 100 μL assay diluent RD1-86 is added to each well. After that, 50 μL of sample (plasma) is added. The ELISA plate is incubated for 2 h at room temperature, 500 rpm. All wells are washed 4 times with wash buffer and 200 μL IL-8 conjugate is added to each well. After incubation for 3 h at room temperature, the wells are washed 4 times with wash buffer and 100 μL working glo reagent is added to each well. The ELISA plate is incubated for 5 min at room temperature (protected from light). Luminescence is measured (0.5 s/well read time).

### 2.4.2 IL-6 Stimulation Protocol

In addition, a flow cytometry analysis was performed to establish JAK1 over JAK2 compound selectivity ex vivo using human whole blood. Therefore, blood was taken from human volunteers who gave informed consent. Blood was then equilibrated for 30 min at 37° C. under gentle rocking, then aliquoted in Eppendorf tubes. Compound was added at different concentrations and incubated at 37° C. for 30 min under gentle rocking and subsequently stimulated for 20 min at 37° C. under gentle rocking with interleukin 6 (IL-6) for JAK1-dependent pathway stimulation or GM-CSF for JAK2-dependent pathway stimulation. Phospho-STAT1 and phospho-STAT5 were then evaluated using FACS analysis.

### 2.4.2.1 PhosphoSTAT1 Assays

For IL-6-stimulated increase of Signal Transducers and Activators of Transcription 1 (pSTAT1) phosphorylation in white blood cell, human whole blood, drawn from human volunteers who gave informed consent, was ex vivo treated with the compound for 30 min and subsequently stimulated for 20 min with IL-6. The increase in phosphorylation of STAT1 by IL-6 in lymphocytes was measured using anti phospho-STAT1 antibody by FACS.

The 5× Lyse/Fix buffer (BD PhosFlow, Cat. No 558049) was diluted 5-fold with distilled water and pre-warmed at 37° C. The remaining diluted Lyse/Fix buffer was discarded.

10 μg rhIL-6 (R&D Systems, Cat No 206-IL) was dissolved in 1 mL of PBS 0.1% BSA to obtain a 10 μg/mL stock solution. The stock solution was aliquoted and stored at −80° C.

A 3-fold dilution series of the compound was prepared in DMSO (10 mM stock solution). Control-treated samples received DMSO instead of compound. All samples were incubated with a 1% final DMSO concentration.

2.4.2.1.1 Incubation of Blood with Compound and Stimulation with IL-6

Human blood was collected in heparinized tubes. The blood was divided in aliquots of 148.5 μL. Then, 1.5 μL of the test compound dilution was added to each blood aliquot and the blood samples were incubated for 30 min at 37° C. under gentle rocking. IL-6 stock solution (1.5 μL) was d added to the blood samples (final concentration 10 ng/mL) and samples were incubated at 37° C. for 20 min under gentle rocking.

### 2.4.2.1.2 White Blood Cell Preparation and CD4 Labeling

At the end of the stimulation period, 3 mL of 1× pre-warmed Lyse/Fix buffer was immediately added to the blood samples, vortexed briefly and incubated for 15 min at 37° C. in a water bath in order to lyse red blood cells and fix leukocytes, then frozen at −80° C. until further use.

For the following steps, tubes were thawed at 37° C. for approximately 20 min and centrifuged for 5 min at 400×g at 4° C. The cell pellet was washed with 3 mL of cold 1×PBS, and after centrifugation the cell pellet was resuspended in 100 μL of PBS containing 3% BSA. FITC-conjugated anti-CD4 antibody or control FITC-conjugated isotype antibody were added and incubated for 20 min at room temperature, in the dark.

2.4.2.1.3 Cell Permeabilization and Labeling with Anti Phospho-STAT1 Antibody

After washing cells with 1×PBS, the cell pellet was resuspended in 100 μL of ice-cold 1×PBS and 900 μL ice-cold 100% MeOH was added. Cells were then incubated at 4° C. for 30 min for permeabilization.

Permeabilized cells were then washed with 1×PBS containing 3% BSA and finally resuspended in 80 μL of 1×PBX containing 3% BSA.

20 μL of PE mouse anti-STAT1 (pY701) or PE mouse IgG2aκ isotype control antibody (BD Biosciences, Cat. No 612564 and 559319, respectively) were added and mixed, then incubated for 30 min at 4° C., in the dark.

Cells are then washed once with 1×PBS and analyzed on a FACSCanto II flow cytometer (BD Biosciences).

### 2.4.2.1.4 Fluorescence Analysis on FACSCanto II

50,000 total events were counted and Phospho-STAT1 positive cells were measured after gating on CD4+ cells, in the lymphocyte gate. Data were analyzed using the FACSDiva software and the percentage inhibition of IL-6 stimulation calculated on the percentage of positive cells for phospho-STAT1 on CD4+ cells.

### 2.4.2.2 Phospho-STAT5 Assay

For GM-CSF-stimulated increase of Signal Transducers and Activators of Transcription 5 (pSTAT5) phosphorylation in white blood cell, human whole blood, drawn from human volunteers who gave informed consent, is ex vivo treated with compound for 30 min and subsequently stimulated for 20 min with GM-CSF. The increase in phosphorylation of STATS by GM-CSF in monocytes is measured using an anti phospho-STAT5 antibody by FACS.

The 5× Lyse/Fix buffer (BD PhosFlow, Cat. No 558049) is diluted 5-fold with distilled water and pre-warmed at 37° C. Remaining diluted Lyse/Fix buffer is discarded.

10 μg rhGM-CSF (AbCys S.A. Cat No P300-03) is dissolved in 100 μL of PBS 0.1% BSA to obtain a 100 μg/mL stock solution. The stock solution is stored aliquoted at −80° C.

A 3-fold dilution series of the compound is prepared in DMSO (10 mM stock solution). Control-treated samples receive DMSO without the test compound. All samples are incubated with a 1% final DMSO concentration.

2.4.2.2.1 Incubation of Blood with Compound and Stimulation with GM-CSF

Human blood is collected in heparinized tubes. The blood is divided in aliquots of 148.5 μL. Then, 1.5 μL of compound dilution is added to each aliquot and the blood samples are incubated for 30 min at 37° C. under gentle rocking. GM-CSF stock solution (1.5 μL) is added to the blood samples (final concentration 20 pg/mL) and samples are incubated at 37° C. for 20 min under gentle rocking.

### 2.4.2.2.2 White Blood Cell Preparation and CD14 Labeling

At the end of the stimulation period, 3 mL of 1× pre-warmed Lyse/Fix buffer is immediately added to the blood samples, vortexed briefly and incubated for 15 min at 37° C. in a water bath in order to lyse red blood cells and fix leukocytes, then frozen at −80° C. until further use.

For the following steps, tubes are thawed at 37° C. for approximately 20 min and centrifuged for 5 min at 400×g at 4° C. The cell pellet is washed with 3 mL of cold 1×PBS, and after centrifugation the cell pellet is resuspended in 100 μL of PBS containing 3% BSA. FITC mouse anti-CD14 antibody (BD Biosciences, Cat. No 345784) or control FITC mouse IgG2bκ isotype antibody (BD Biosciences, Cat. No 555057) are added and incubated for 20 min at room temperature, in the dark.

2.4.2.2.3 Cell Permeabilization and Labeling with Anti Phospho-STATS Antibody

After washing cells with 1×PBS, the cell pellet is resuspended in 100 μL of ice-cold 1×PBS and 900 μL of ice-cold 100% MeOH is added. Cells are then incubated at 4° C. for 30 min for permeabilization.

Permeabilized cells are then washed with 1×PBS containing 3% BSA and finally resuspended in 80 μL of 1×PBX containing 3% BSA.

20 μL of PE mouse anti-STATS (pY694) or PE mouse IgG1κ isotype control antibody (BD Biosciences, Cat. No 612567 and 554680, respectively) are added, mixed then incubated for 30 min at 4° C., in the dark.

Cells are then washed once with 1×PBS and analyzed on a FACSCanto II flow cytometer (BD Biosciences).

### 2.4.2.2.4 Fluorescence Analysis on FACSCanto II

50,000 total events are counted and Phospho-STAT5 positive cells are measured after gating on CD14+ cells. Data are analyzed using the FACSDiva software and correspond to the percentage of inhibition of GM-CSF stimulation calculated on the percentage of positive cells for phosphor-STATS on CD14+ cells.

### Example 2.5

### CTLL2 Viability Assay

The CTLL-2 cell line is an IL-2 dependent mouse T cell line that constitutively express IL2 receptors and depends entirely on the presence of IL2 for its growth (Haan et al., 2011 Chem. Biol., 18(3):314-23). Since IL-2 signaling depends on the activity of the JAK-kinase family member JAK1 and JAK3, the proliferation and survival of CTLL-2 cells can be used to test the JAK1/JAK3 inhibitory capacity of the compounds of the invention.

The protocol describes the methods to analyse the activity of compounds on their ability to sustain the IL2-dependent viability of CTLL2.

CTLL2 cells were cultured in RPMI1640 medium (Life Technologies Cat no 21875-034), with 10% fetal bovine serum (FBS, HiClone SV30160.03, 1% penicillin/streptomycin and 10% of the IL-2 culture supplement T_STIM with ConA (BD Biosciences no 354115).

CTLL cells are seeded at 1000 cells per well of a white 384 well plate (Greiner, 781080) in 20 μL medium RPMI1640.

To the wells, 10 μL of diluted compound (or controls) was added. Negative control was a DMSO dilution, positive control was Incyte INCB018424 at 10 μM. Final DMSO concentration was 0.1%.

The plates were incubated at 37° C. for 24 h and then the ATP contert as measured using ATP-lite (Perkin Elmer, cat no 6016739). For this, 30 μL ATP-lite solution was added to each well, and after 2 min shaking and another 8 min incubation at room temp in the dark, bioluminescence was measured in a PerkinElmer Envision multireader equipped for luminescence.

TABLE VIII
CTLL2 IC₅₀ of selected compounds of the invention

| Cpd # | CTLL2 |
|---|---|
| 81 | * |
| 93 | * |
| 95 | * |
| 101 | * |
| 115 | * |
| 121 | * |
| 137 | * |
| 151 | * |
| 167 | * |
| 176 | ** |
| 182 | * |
| 186 | * |
| 190 | * |
| 194 | * |
| 195 | * |
| 197 | ** |
| 199 | * |
| 201 | ** |
| 210 | * |
| 214 | ** |
| 227 | * |
| 228 | *** |
| 229 | * |
| 243 | * |
| 244 | * |
| 245 | * |
| 248 | * |
| 253 | * |
| 254 | * |
| 256 | * |
| 261 | ** |
| 262 | * |
| 274 | * |
| 280 | * |
| 307 | * |
| 319 | * |
| 323 | * |
| 325 | * |
| 327 | ** |
| 328 | * |
| 329 | ** |
| 333 | * |
| 334 | * |
| 337 | * |
| 341 | * |
| 342 | * |
| 343 | * |
| 344 | * |
| 346 | * |
| 347 | * |
| 349 | * |
| 350 | * |
| 351 | * |
| 352 | * |
| 353 | ** |
| 354 | ** |
| 356 | ** |
| 357 | * |
| 358 | * |
| 362 | * |
| 363 | * |
| 364 | * |
| 365 | * |
| 369 | * |
| 370 | * |
| 372 | * |
| 373 | * |
| 375 | * |
| 376 | * |
| 377 | * |
| 378 | * |
| 379 | * |
| 380 | * |
| 381 | * |
| 383 | * |
| 384 | * |
| 385 | * |
| 387 | * |
| 388 | * |
| 389 | * |
| 390 | * |
| 391 | * |
| 392 | * |
| 393 | * |
| 394 | * |
| 395 | * |
| 396 | * |
| 397 | * |
| 398 | * |
| 400 | * |
| 402 | * |
| 404 | * |
| 405 | * |
| 406 | ** |
| 407 | * |
| 409 | * |
| 410 | * |
| 411 | * |
| 413 | * |
| 414 | * |
| 415 | * |
| 419 | * |
| 420 | * |
| 421 | * |
| 422 | * |
| 423 | * |
| 424 | * |
| 425 | * |
| 426 | * |
| 429 | * |
| 430 | * |
| 432 | * |
| 433 | * |
| 434 | * |
| 435 | * |
| 436 | * |
| 437 | * |
| 438 | * |
| 439 | * |
| 440 | * |
| 441 | * |
| 445 | * |
| 446 | * |
| 447 | * |
| 448 | * |
| 449 | * |
| 450 | * |
| 451 | * |
| 452 | ** |
| 453 | * |

* >1001 nM
** 501-1000 nM
*** 1-500 nM

### Example 2.6

### BA/F3 Viability Assay

The BA/F3 cell line is an IL-3 dependent mouse pro-B-cell line that undergoes cell death upon IL-3 withdrawal (Collins, Marvel et al. 1992). Since IL-3 signaling depends on the activity of the JAK kinase family member JAK2 only (Reddy, Korapati et al. 2000), the proliferation and survival of BA/F3 cells can be used to test the JAK2 inhibitory capacity of a compound of the invention.

BA/F3 cells were cultured in RPMI1640 medium (Life Technologies Cat no 21875-034), with 10% fetal bovine serum (FBS, HiClone SV30160.03, 1% pen/strep and 10 ng/mL IL-3 (peprotech, no 213-13) BA/F3 cells were seeded at 1500 cells per well of a white 384 well plate (Greiner, 781080) in 20 μL medium. To each wells, 10 μL of diluted compound (or controls) is added. Negative control was a DMSO dilution, positive control was Tofacitinib (CP-690550) at 10 μM. Final DMSO concentration was 0.1%.

The plates were incubated at 37° C. for 48 h and then the ATP contert was measured using ATP-lite (Perkin Elmer, cat no 6016739). For this, 30 μL ATP-lite solution was added to each well, and after 2 min shaking and another 8 min incubation at room temp in the dark, bioluminescence is measured in a PerkinElmer Envision mutireader equipped for luminescence.

TABLE IX
BA/F3 IC₅₀ of selected compounds of the invention

| Cpd # | BA/F3 |
|---|---|
| 81 | * |
| 93 | * |
| 95 | * |
| 101 | * |
| 115 | * |
| 121 | * |
| 133 | * |
| 151 | ** |
| 167 | * |
| 176 | ** |
| 182 | * |
| 186 | ** |
| 190 | ** |
| 194 | ** |
| 195 | ** |
| 197 | ** |
| 199 | ** |
| 210 | *** |
| 214 | *** |
| 225 | ** |
| 227 | * |
| 228 | * |
| 229 | ** |
| 243 | ** |
| 244 | ** |
| 245 | ** |
| 248 | ** |
| 253 | * |
| 254 | * |
| 256 | * |
| 261 | ** |
| 262 | ** |
| 269 | * |
| 274 | ** |
| 280 | * |
| 307 | * |
| 319 | ** |
| 323 | * |
| 325 | * |
| 327 | ** |
| 328 | * |
| 329 | * |
| 333 | * |
| 334 | * |
| 337 | * |
| 341 | * |
| 342 | * |
| 344 | * |
| 346 | * |
| 347 | * |
| 349 | ** |
| 350 | * |
| 351 | * |
| 352 | * |
| 353 | ** |
| 354 | ** |
| 356 | ** |
| 357 | ** |
| 358 | * |
| 362 | * |
| 363 | ** |
| 364 | * |
| 369 | ** |
| 370 | ** |
| 375 | ** |
| 376 | ** |
| 377 | * |
| 378 | * |
| 379 | * |
| 380 | ** |
| 381 | * |
| 383 | ** |
| 385 | ** |
| 387 | * |
| 388 | ** |
| 389 | * |
| 390 | * |
| 391 | ** |
| 392 | * |
| 393 | * |
| 394 | ** |
| 395 | * |
| 396 | * |
| 397 | ** |
| 398 | ** |
| 400 | * |
| 402 | ** |
| 404 | * |
| 405 | ** |
| 406 | ** |
| 407 | * |
| 409 | * |
| 410 | * |
| 411 | ** |
| 413 | ** |
| 429 | * |
| 430 | ** |

* >5001 nM
** 1001-5000 nM
*** 1-1000 nM

### Example 3

### In Vivo Models

### Example 3.1

### CIA Model

### 3.1.1 Materials

Completed Freund's adjuvant (CFA) and incomplete Freund's adjuvant (IFA) were purchased from Difco. Bovine collagen type II (CII), lipopolysaccharide (LPS), and Enbrel was obtained from Chondrex (Isle d'Abeau, France); Sigma (P4252, L′Isle d'Abeau, France), Whyett (25 mg injectable syringe, France) Acros Organics (Palo Alto, Calif.), respectively. All other reagents used were of reagent grade and all solvents were of analytical grade.

### 3.1.2 Animals

Dark Agouti rats (male, 7-8 weeks old) were obtained from Harlan Laboratories (Maison-Alfort, France). Rats were kept on a 12 hr light/dark cycle (0700-1900). Temperature was maintained at 22° C., and food and water were provided ad libitum.

### 3.1.3 Collagen Induced Arthritis (CIA)

One day before the experiment, CII solution (2 mg/mL) was prepared with 0.05 M acetic acid and stored at 4° C. Just before the immunization, equal volumes of adjuvant (IFA) and CII were mixed by a homogenizer in a pre-cooled glass bottle in an ice water bath. Extra adjuvant and prolonged homogenization may be required if an emulsion is not formed. 0.2 mL of the emulsion was injected intradermally at the base of the tail of each rat on day 1, a second booster intradermal injection (CII solution at 2 mg/mL in CFA 0.1 mL saline) was performed on day 9. This immunization method was modified from published methods (Sims et al, 2004; Jou et al., 2005).

### 3.1.4 Study Design

The therapeutic effects of the compounds were tested in the rat CIA model. Rats were randomly divided into equal groups and each group contained 10 rats. All rats were immunized on day 1 and boosted on day 9. Therapeutic dosing lasted from day 16 to day 30. The negative control group was treated with vehicle (MC 0.5%) and the positive control group with Enbrel (10 mg/kg, 3× week. s.c.). A compound of interest was typically tested at 3 doses, e.g. 3, 10, 30 mg/kg, p.o.

### 3.1.5 Clinical Assessment of Arthritis

Arthritis is scored according to the method of Khachigian 2006, Lin et al 2007 and Nishida et al. 2004). The swelling of each of the four paws is ranked with the arthritic score as follows: 0—no symptoms; 1—mild, but definite redness and swelling of one type of joint such as the ankle or wrist, or apparent redness and swelling limited to individual digits, regardless of the number of affected digits; 2—moderate redness and swelling of two or more types of joints; 3—severe redness and swelling of the entire paw including digits; 4—maximally inflamed limb with involvement of multiple joints (maximum cumulative clinical arthritis score 16 per animal) (Nishida et al., 2004).

To permit the meta-analysis of multiple studies the clinical score values were normalised as follows:

AUC of Clinical Score (AUC Score):

The area under the curve (AUC) from day 1 to day 14 was calculated for each individual rat. The AUC of each animal was divided by the average AUC obtained for the vehicle in the study from which the data on that animal was obtained and multiplied by 100 (i.e. the AUC was expressed as a percentage of the average vehicle AUC per study).

Clinical Score Increase from Day 1 to Day 14 (End Point Score):

The clinical score difference for each animal was divided by the average clinical score difference obtained for the vehicle in the study from which the data on that animal was obtained and multiplied by 100 (i.e. the difference was expressed as a percentage of the average clinical score difference for the vehicle per study).

3.1.6 Change in Body Weight (%) after Onset of Arthritis

Clinically, body weight loss is associated with arthritis (Shelton et al., 2005; Rall, 2004; Walsmith et al., 2004). Hence, changes in body weight after onset of arthritis can be used as a non-specific endpoint to evaluate the effect of therapeutics in the rat model. The change in body weight (%) after onset of arthritis was calculated as follows:

$$Mice \text{:} \frac{Body {Weight}_{( week 6 )} - Body {Weight}_{( week 5 )}}{Body {Weight}_{( week 5 )}} \times 100 \%$$

$$Rats \text{:} \frac{Body {Weight}_{( week 4 )} - Body {Weight}_{( week 3 )}}{Body {Weight}_{( week 3 )}} \times 100 \%$$

### 3.1.7 Radiology

X-ray photos were taken of the hind paws of each individual animal. A random blind identity number was assigned to each of the photos, and the severity of bone erosion was ranked by two independent scorers with the radiological Larsen's score system as follows: 0—normal with intact bony outlines and normal joint space; 1—slight abnormality with any one or two of the exterior metatarsal bones showing slight bone erosion; 2—definite early abnormality with any three to five of the exterior metatarsal bones showing bone erosion; 3—medium destructive abnormality with all the exterior metatarsal bones as well as any one or two of the interior metatarsal bones showing definite bone erosions; 4—severe destructive abnormality with all the metatarsal bones showing definite bone erosion and at least one of the inner metatarsal joints completely eroded leaving some bony joint outlines partly preserved; 5—mutilating abnormality without bony outlines. This scoring system is a modification from Salvemini et al., 2001; Bush et al., 2002; Sims et al., 2004; Jou et al., 2005.

### 3.1.8 Histology

After radiological analysis, the hind paws of mice were fixed in 10% phosphate-buffered formalin (pH 7.4), decalcified with rapid bone decalcifiant for fine histology (Laboratories Eurobio) and embedded in paraffin. To ensure extensive evaluation of the arthritic joints, at least four serial sections (5 μm thick) were cut and each series of sections were 100 μm in between. The sections were stained with hematoxylin and eosin (H&E). Histologic examinations for synovial inflammation and bone and cartilage damage were performed double blind. In each paw, four parameters were assessed using a four-point scale. The parameters were cell infiltration, pannus severity, cartilage erosion and bone erosion. Scoring was performed according as follows: 1—normal, 2—mild, 3—moderate, 4—marked. These four scores are summed together and represented as an additional score, namely the ‘RA total score’.

### 3.1.9 Micro-Computed Tomography (μCT) Analysis of Calcaneus (Heel Bone):

Bone degradation observed in RA occurs especially at the cortical bone and can be revealed by μCT analysis (Sims N A et al., Arthritis Rheum. 50 (2004) 2338-2346: Targeting osteoclasts with zoledronic acid prevents bone destruction in collagen-induced arthritis; Oste L et al., ECTC Montreal 2007: A high throughput method of measuring bone architectural disturbance in a murine CIA model by micro-CT morphometry). After scanning and 3D volume reconstruction of the calcaneus bone, bone degradation is measured as the number of discrete objects present per slide, isolated in silico perpendicular to the longitudinal axis of the bone. The more the bone is degraded, the more discrete objects are measured. 1000 slices, evenly distributed along the calcaneus (spaced by about 10.8 μm), are analyzed.

### 3.1.10 Steady State PK

At day 7 or 11, blood samples were collected at the retro-orbital sinus with lithium heparin as anti-coagulant at the following time points: predose, 1, 3 and 6 h. Whole blood samples were centrifuged and the resulting plasma samples were stored at −20° C. pending analysis. Plasma concentrations of each test compound were determined by an LC-MS/MS method in which the mass spectrometer was operated in positive electrospray mode. Pharmacokinetic parameters were calculated using Winnonlin® (Pharsight®, United States) and it was assumed that the predose plasma levels were equal to the 24 h plasma levels.

### Example 3.2

### Septic Shock Model

Injection of lipopolysaccharide (LPS) induces a rapid release of soluble tumour necrosis factor (TNF-alpha) into the periphery. This model is used to analyse prospective blockers of TNF release in vivo.

Six BALB/cJ female mice (20 g) per group are treated at the intended dosing once, po. Thirty min later, LPS (15 μg/kg; E. Coli serotype 0111:B4) is injected ip. Ninety min later, mice are euthanized and blood is collected. Circulating TNF alpha levels are determined using commercially available ELISA kits. Dexamethasone (5 μg/kg) is used as a reference anti-inflammatory compound.

### Example 3.3

### MAB Model

The MAB model allows a rapid assessment of the modulation of an RA-like inflammatory response by therapeutics (Kachigian LM. Nature Protocols (2006) 2512-2516: Collagen antibody-induced arthritis). DBA/J mice are injected i.v. with a cocktail of mAbs directed against collagen II. One day later, compound treatment is initiated (vehicle: 10% (v/v) HPβCD). Three days later, mice receive an i.p. LPS injection (50 mg/mouse), resulting in a fast onset of inflammation. Compound treatment is continued until 10 days after the mAb injection. Inflammation is read by measuring paw swelling and recording the clinical score of each paw. The cumulative clinical arthritis score of four limbs is presented to show the severity of inflammation. A scoring system is applied to each limb using a scale of 0-4, with 4 being the most severe inflammation.
0 Symptom free
1 Mild, but definite redness and swelling of one type of joint such as the ankle or wrist, or apparent redness and swelling limited to individual digits, regardless of the number of affected digits
2 Moderate redness and swelling of two or more types of joints
3 Severe redness and swelling of the entire paw including digits
4 Maximally inflamed limb with involvement of multiple joints

### Example 3.4

### Oncology Models

In vivo models to validate efficacy of small molecules towards JAK2-driven myeloproliferative diseases are described by Wernig et al. Cancer Cell 13, 311, 2008 and Geron et al. Cancer Cell 13, 321, 2008.

### Example 3.5

### Mouse IBD Model

In vitro and in vivo models to validate efficacy of small molecules towards IBD are described by Wirtz et al. 2007.

### Example 3.6

### Mouse Asthma Model

In vitro and in vivo models to validate efficacy of small molecules towards asthma are described by Nials et al., 2008; Ip et al. 2006; Pernis et al., 2002; Kudlacz et al., 2008.

### Example 4

### Pharmacokinetic, DMPK and Toxicity Assays

### Example 4.1

### Thermodynamic Solubility

A solution of 1 mg/mL of the test compound is prepared in a 0.2M phosphate buffer pH 7.4 or a 0.1M citrate buffer pH 3.0 at room temperature in a glass vial.

The samples are rotated in a Rotator drive STR 4 (Stuart Scientific, Bibby) at speed 3.0 at room temperature for 24 h.

After 24 h, 800 μL of the sample is transferred to an eppendorf tube and centrifuged 5 min at 14000 rpm. 200 μL of the supernatant of the sample is then transferred to a MultiscreenR Solubility Plate (Millipore, MSSLBPC50) and the supernatant is filtered (10-12″ Hg) with the aid of a vacuum manifold into a clean Greiner polypropylene V-bottom 96 well plate (Cat no. 651201). 5 μL of the filtrate is diluted into 95 μL (F20) of the same buffer used to incubate in the plate containing the standard curve (Greiner, Cat no. 651201).

The standard curve for the compound is prepared freshly in DMSO starting from a 10 mM DMSO stock solution diluted factor 2 in DMSO (5000 μM) and then further diluted in DMSO up to 19.5 μM. 3 μL of the dilution series as from 5000 μM is then transferred to a 97 μL acetonitrile-buffer mixture (50/50). The final concentration range is 2.5 to 150 μM.

The plate is sealed with sealing mats (MA96RD-04S, www.kinesis.co.uk) and samples are measured at room temperature on LCMS (ZQ 1525 from Waters) under optimized conditions using Quanoptimize to determine the appropriate mass of the molecule.

The samples are analyzed on LCMS with a flow rate of 1 mL/min. Solvent A is 15 mM ammonia and solvent B is acetonitrile. The sample is run under positive ion spray on an XBridge C18 3.5 μM (2.1×30 mm) column, from Waters. The solvent gradient has a total run time of 2 min and ranges from 5% B to 95% B.

Peak areas are analyzed with the aid of Masslynx software package and peak areas of the samples are plotted against the standard curve to obtain the solubility of the compound.

Solubility values are reported in μM or μg/mL.

### Example 4.2

### Aqueous Solubility

### 4.2.1: Aqueous Solubility 2% DMSO Procedure

Starting from a 10 mM stock in DMSO, a serial dilution of the compound is prepared in DMSO. The dilution series is transferred to a 96 NUNC Maxisorb plate F-bottom (Cat no. 442404) and 0.2M phosphate buffer pH7.4 or 0.1M citrate buffer pH 3.0 at room temperature is added.

The final concentration ranged from 200 μM to 2.5 μM in 5 equal dilution steps. The final DMSO concentration does not exceed 2%. 200 μM Pyrene is added to the corner points of each 96 well plate and served as a reference point for calibration of Z-axis on the microscope.

The assay plates are sealed and incubated for 1 hr at 37° C. while shaking at 230 rpm. The plates are then scanned under a white light microscope, yielding individual pictures of the precipitate per concentration. The precipitate is analyzed and converted into a number which is plotted onto a graph. The first concentration at which the compound appears completely dissolved is the concentration reported, however the true concentration lies somewhere between this concentration and one dilution step higher.

Solubility values measured according to this protocol are reported in μg/mL.

4.2.1: Aqueous Solubility 3% DMSO Procedure

Starting from a 10 mM stock in DMSO, a serial dilution of the compound is prepared in DMSO. The dilution series is transferred to a 96 NUNC Maxisorb plate F-bottom (Cat no. 442404) and 0.1M phosphate buffer pH7.4 or 0.1M citrate buffer pH3.0 at room temperature is added.

The final concentration ranges from 300 μM to 18.75 μM in 5 equal dilution steps. The final DMSO concentration does not exceed 3%. 200 μM Pyrene is added to the corner points of each 96 well plate and serves as a reference point for calibration of Z-axis on the microscope.

The assay plates are sealed and incubated for 1 h at 37° C. while shaking at 230 rpm. The plates are then scanned under a white light microscope, yielding individual pictures of the precipitate per concentration. The precipitate is analyzed and converted into a number with a software tool which can be plotted onto a graph. The first concentration at which the compound appears completely dissolved is the concentration reported; however the true concentration lies somewhere between this concentration and one dilution step higher.

Solubility values measured according to this protocol are reported in μg/mL.

### Example 4.3

### Plasma Protein Binding (Equilibrium Dialysis)

A 10 mM stock solution of the compound in DMSO is diluted with a factor 5 in DMSO. This solution is further diluted in freshly thawed human, rat, mouse or dog plasma (BioReclamation INC) with a final concentration of 10 μM and final DMSO concentration of 0.5% (5.5 μL in 1094.5 μL plasma in a PP-Masterblock 96well (Greiner, Cat no. 780285))

A Pierce Red Device plate with inserts (ThermoScientific, Cat no. 89809) is prepared and filled with 750 μL PBS in the buffer chamber and 500 μL of the spiked plasma in the plasma chamber. The plate is incubated for 4 h at 37° C. while shaking at 230 rpm. After incubation, 120 μL of both chambers is transferred to 360 μL acetonitrile in a 96-well round bottom, PP deep-well plates (Nunc, Cat no. 278743) and sealed with an aluminum foil lid. The samples are mixed and placed on ice for 30 min. This plate is then centrifuged 30 min at 1200rcf at 4° C. and the supernatant is transferred to a 96 v-bottom PP plate (Greiner, 651201) for analysis on LCMS.

The plate is sealed with sealing mats (MA96RD-04S) of www.kinesis.co.uk and samples are measured at room temperature on LCMS (ZQ 1525 from Waters) under optimized conditions using Quanoptimize to determine the appropriate mass of the molecule.

The samples are analyzed on LCMS with a flow rate of 1 mL/min. Solvent A is 15 mM ammonia and solvent B is acetonitrile. The sample is run under positive ion spray on an XBridge C18 3.5 μM (2.1×30 mm) column, from Waters. The solvent gradient has a total run time of 2 min and ranges from 5% B to 95% B.

Peak area from the compound in the buffer chamber and the plasma chamber are considered to be 100% compound. The percentage bound to plasma is derived from these results and is reported as percentage bound to plasma.

The solubility of the compound in the final test concentration in PBS is inspected by microscope to indicate whether precipitation is observed or not.

### Example 4.4

### Microsomal Stability

### 4.4.1 Microsomal Stability 1 h Incubation Procedure

A 10 mM stock solution of compound in DMSO is diluted 1000 fold in a 182 mM phosphate buffer pH7.4 in a 96 deep well plate (Greiner, Cat no. 780285) and pre-incubated at 37° C.

40 μL of deionised water is added to a well of a polypropylene Matrix 2D barcode labelled storage tube (Thermo Scientific) and pre-incubated at 37° C.

A Glucose-6-phophate-dehydrogenase (G6PDH) working stock solution is prepared in 182 mM phosphate buffer pH7.4 and placed on ice before use. A co-factor containing MgCl₂, glucose-6-phosphate and NADP+ is prepared in deionised water and placed on ice before use.

A final working solution containing liver microsomes (Xenotech) of a species of interest (human, mouse, rat, dog), previously described G6PDH and co-factors is prepared and this mix is incubated for no longer than 20 min at room temperature.

30 μL of the pre-heated compound dilution is added to 40 μL of pre-heated water in the Matrix tubes and 30 μL of the microsomal mix is added. Final reaction concentrations are 3 μM compound, 1 mg microsomes, 0.4 U/mL GDPDH, 3.3 mM MgCl₂, 3.3 mM glucose-6-phosphate and 1.3 mM NADP+.

To measure percentage remaining of compound at time zero MeOH or ACN is added (1:1) to the well before adding the microsomal mix. The plates are sealed with Matrix Sepra Seals™ (Matrix, Cat. No. 4464) and shaken for a few seconds ensure complete mixing of all components.

The samples which were not stopped are incubated at 37° C., 300 rpm and after 1 hr of incubation the reaction is stopped with MeOH or ACN (1:1).

After stopping the reaction the samples are mixed and placed on ice for 30 min to precipitate the proteins. The plates are then centrifuged 30 min at 1200rcf at 4° C. and the supernatant is transferred to a 96 v-bottom PP plate (Greiner, 651201) for analysis on LCMS.

These plates are sealed with sealing mats (MA96RD-04S) of www.kinesis.co.uk and samples are measured at room temperature on LCMS (ZQ 1525 from Waters) under optimized conditions using Quanoptimize to determine the appropriate mass of the parent molecule.

The samples are analyzed on LCMS with a flow rate of 1 mL/min. Solvent A is 15 mM ammonia and solvent B is MeOH or acetonitrile, depending on the stop solution used. The samples are run under positive ion spray on an XBridge C18 3.5 μM (2.1×30 mm) column, from Waters. The solvent gradient had a total run time of 2 min and ranges from 5% B to 95% B.

Peak area from the parent compound at time 0 is considered to be 100% remaining. The percentage remaining after 1 hr incubation is calculated from time 0 and is calculated as the percentage remaining. The solubility of the compound in the final test concentration in buffer is inspected by microscope and results are reported.

The data on microsomal stability are expressed as a percentage of the total amount of compound remaining after 60 min.

### 4.4.2 Microsomal Stability 30 Min Incubation Procedure

A 10 mM stock solution of compound in DMSO is diluted to 6 μM in a 105 mM phosphate buffer, pH7.4 in a 96 deep well plate (Greiner, Cat no. 780285) and pre-warmed at 37° C.

A Glucose-6-phosphate-dehydrogenase (G6PDH, Roche, 10127671001) working stock solution of 700 U/mL is diluted with a factor 1:700 in a 105 mM phosphate buffer, pH7.4. A co-factor mix containing 0.528M MgCl₂.6H₂O (Sigma, M2670), 0.528M glucose-6-phosphate (Sigma, G-7879) and 0.208M NADP+(Sigma, N-0505) is diluted with a factor 1:8 in a 105 mM phosphate buffer, pH7.4.

A working solution is made containing 1 mg/mL liver microsomes (Provider, Xenotech) of the species of interest (human, mouse, rat, dog . . . ), 0.8 U/mL G6PDH and co-factor mix (6.6 mM MgCl₂, 6.6 mM glucose-6-phosphate, 2.6 mM NADP+). This mix is pre-incubated for 15 min, but never more than 20 min, at room temperature.

After pre-incubation, compound dilution and the mix containing the microsomes, are added together in equal amount and incubated for 30 min at 300 rpm. For the time point of 0 min, two volumes of MeOH are added to the compound dilution before the microsome mix is added. The final concentration during incubation are: 3 μM test compound or control compound, 0.5 mg/mL microsomes, 0.4 U/mL G6PDH, 3.3 mM MgCl₂, 3.3 mM glucose-6-phosphate and 1.3 mM NaDP+.

After 30 min of incubation, the reaction is stopped with 2 volumes of MeOH.

Of both time points, samples are mixed, centrifuged and the supernatant is harvested for analysis on LC-MS/MS. The instrument responses (i.e. peak heights) are referenced to the zero time-point samples (as 100%) in order to determine the percentage of compound remaining Standard compounds Propanolol and Verapamil are included in the assay design.

The data on microsomal stability are expressed as a percentage of the total amount of compound remaining after 30 min.

### Example 4.5

### Caco2 Permeability

Bi-directional Caco-2 assays are performed as described below. Caco-2 cells are obtained from European Collection of Cell Cultures (ECACC, cat 86010202) and used after a 21 day cell culture in 24-well Transwell plates (Fisher TKT-545-020B).

2×10⁵ cells/well are seeded in plating medium consisting of DMEM+GlutaMAXI+1% NEAA+10% FBS (FetalClone II)+1% Pen/Strep. The medium is changed every 2-3 days.

Test and reference compounds (propranolol and rhodamine123 or vinblastine, all purchased from Sigma) are prepared in Hanks' Balanced Salt Solution containing 25 mM HEPES (pH7.4) and added to either the apical (125 μL) or basolateral (600 μL) chambers of the Transwell plate assembly at a concentration of 10 μM with a final DMSO concentration of 0.25%.

50 μM Lucifer Yellow (Sigma) is added to the donor buffer in all wells to assess integrity of the cell layers by monitoring Lucifer Yellow permeation. As Lucifer Yellow (LY) cannot freely permeate lipophilic barriers, a high degree of LY transport indicates poor integrity of the cell layer.

After a 1 hr incubation at 37° C. while shaking at an orbital shaker at 150 rpm, 70 μL aliquots are taken from both apical (A) and basal (B) chambers and added to 100 μL1 50:50 acetonitrile:water solution containing analytical internal standard (0.5 μM carbamazepine) in a 96 well plate.

Lucifer yellow is measured with a Spectramax Gemini XS (Ex 426 nm and Em 538 nm) in a clean 96 well plate containing 150 μL of liquid from basolateral and apical side.

Concentrations of compound in the samples are measured by high performance liquid-chromatography/mass spectroscopy (LC-MS/MS).

Apparent permeability (Pₐₚₚ) values are calculated from the relationship:

Pₐₚₚ=[compound]_{acceptor final}×V_{acceptor}/([compound]_{donor initial}×V_{donor})/T_{inc}×V_{donor}/surface area×60×10⁻⁶cm/s
V=chamber volume
T_{inc}=incubation time.
Surface area=0.33 cm²

The Efflux ratios, as an indication of active efflux from the apical cell surface, are calculated using the ratio of PₐₚₚB>A/PₐₚₚA>B.

The following assay acceptance criteria are used:

Propranolol: Pₐₚₚ (A>B) value≧20(×10⁻⁶ cm/s)

Rhodamine 123 or Vinblastine: Pₐₚₚ(A>B) value<5 (×10⁻⁶ cm/s) with Efflux ratio≧5.

Lucifer yellow permeability: ≦100 nm/s

### Example 4.6

### MDCKII-MDR1 Permeability

MDCKII-MDR1 cells are Madin-Darby canine kidney epithelial cells, over-expressing human multi-drug resistance (MDR1) gene, coding for P-glycoprotein (P-gp). Cells are obtained from Netherlands Cancer Institute and used after a 3-4 day cell culture in 24-well Millicell cell culture insert plates (Millipore, PSRP010R5). Bi-directional MDCKII-MDR1 permeability assay is performed as described below.

3×10⁵ cells/mL (1.2×10⁵ cells/well) are seeded in plating medium consisting of DMEM+1% Glutamax-100+1% Antibiotic/Antimycotic+10% FBS (Biowest, S1810). Cells are left in CO₂ incubator for 3-4 days. The medium is changed 24 h after seeding and on the day of experiment.

Test and reference compounds (amprenavir and propranolol) are prepared in Dulbecco's phosphate buffer saline (D-PBS, pH7.4) and added to either the apical (400 μL) or basolateral (800 μL) chambers of the Millicell cell culture insert plates assembly at a final concentration of 10 μM (0.5 μM in case of amprenavir) with a final DMSO concentration of 1%.

100 μM Lucifer Yellow (Sigma) is added to the all donor buffer solutions, in order to assess integrity of the cell monolayers by monitoring Lucifer Yellow permeation. Lucifer yellow is a fluorescent marker for the paracellular pathway and it is used as an internal control in every monolayer to verify tight junction integrity during the assay.

After a 1 hr incubation at 37° C. while shaking at an orbital shaker at 150 rpm, 75 μL aliquots are taken from both apical (A) and basal (B) chambers and added to 225 μL acetonitrile:water solution (2:1) containing analytical internal standard (10 ng/mL warfarin) in a 96 well plate. Aliquoting is also performed at the beginning of the experiment from donor solutions to obtain initial (Co) concentration.

Concentration of compound in the samples is measured by high performance liquid-chromatography/mass spectroscopy (LC-MS/MS).

Lucifer yellow is measured with a Fluoroscan Ascent FL Thermo Scientific (Ex 485 nm and Em 530 nm) in a 96 well plate containing 150 μL of liquid from all receiver wells (basolateral or apical side).

### Example 4.7

### Microsomal Stability

### 4.7.1 Microsomal Stability 1 h Incubation Procedure

A 10 mM stock solution of compound in DMSO is diluted 1000 fold in a 182 mM phosphate buffer pH7.4 in a 96 deep well plate (Greiner, Cat no. 780285) and pre-incubated at 37° C.

40 μL of deionised water is added to a well of a polypropylene Matrix 2D barcode labelled storage tube (Thermo Scientific) and pre-incubated at 37° C.

A Glucose-6-phophate-dehydrogenase (G6PDH) working stock solution is prepared in 182 mM phosphate buffer pH7.4 and placed on ice before use. A co-factor containing MgCl₂, glucose-6-phosphate and NADP+ is prepared in deionised water and placed on ice before use.

A final working solution containing liver microsomes (Xenotech) of a species of interest (human, mouse, rat, dog), previously described G6PDH and co-factors is prepared and this mix is incubated for no longer than 20 min at room temperature.

30 μL of the pre-heated compound dilution is added to 40 μL of pre-heated water in the Matrix tubes and 30 μL of the microsomal mix is added. Final reaction concentrations are 3 μM compound, 1 mg microsomes, 0.4 U/mL GDPDH, 3.3 mM MgCl₂, 3.3 mM glucose-6-phosphate and 1.3 mM NADP+.

To measure percentage remaining of compound at time zero MeOH or ACN is added (1:1) to the well before adding the microsomal mix. The plates are sealed with Matrix Sepra Seals™ (Matrix, Cat. No. 4464) and shaken for a few seconds ensure complete mixing of all components.

The samples which were not stopped are incubated at 37° C., 300 rpm and after 1 hr of incubation the reaction is stopped with MeOH or ACN (1:1).

After stopping the reaction the samples are mixed and placed on ice for 30 min to precipitate the proteins. The plates are then centrifuged 30 min at 1200rcf at 4° C. and the supernatant is transferred to a 96 v-bottom PP plate (Greiner, 651201) for analysis on LCMS.

These plates are sealed with sealing mats (MA96RD-04S) of www.kinesis.co.uk and samples are measured at room temperature on LCMS (ZQ 1525 from Waters) under optimized conditions using Quanoptimize to determine the appropriate mass of the parent molecule.

The samples are analyzed on LCMS with a flow rate of 1 mL/min. Solvent A is 15 mM ammonia and solvent B is MeOH or acetonitrile, depending on the stop solution used. The samples are run under positive ion spray on an XBridge C18 3.5 μM (2.1×30 mm) column, from Waters. The solvent gradient had a total run time of 2 min and ranges from 5% B to 95% B.

Peak area from the parent compound at time 0 is considered to be 100% remaining. The percentage remaining after 1 hr incubation is calculated from time 0 and is calculated as the percentage remaining. The solubility of the compound in the final test concentration in buffer is inspected by microscope and results are reported.

The data on microsomal stability are expressed as a percentage of the total amount of compound remaining after 60 min.

### 4.7.2 Microsomal Stability 30 min Incubation Procedure

A 10 mM stock solution of compound in DMSO is diluted to 6 μM in a 105 mM phosphate buffer, pH7.4 in a 96 deep well plate (Greiner, Cat no. 780285) and pre-warmed at 37° C.

A Glucose-6-phosphate-dehydrogenase (G6PDH, Roche, 10127671001) working stock solution of 700 U/mL is diluted with a factor 1:700 in a 105 mM phosphate buffer, pH7.4. A co-factor mix containing 0.528M MgCl₂.6H₂O (Sigma, M2670), 0.528M glucose-6-phosphate (Sigma, G-7879) and 0.208M NADP+(Sigma, N-0505) is diluted with a factor 1:8 in a 105 mM phosphate buffer, pH7.4.

A working solution is made containing 1 mg/mL liver microsomes (Provider, Xenotech) of the species of interest (human, mouse, rat, dog . . . ), 0.8 U/mL G6PDH and co-factor mix (6.6 mM MgCl₂, 6.6 mM glucose-6-phosphate, 2.6 mM NADP+). This mix is pre-incubated for 15 min, but never more than 20 min, at room temperature.

After pre-incubation, compound dilution and the mix containing the microsomes, are added together in equal amount and incubated for 30 min at 300 rpm. For the time point of 0 min, two volumes of MeOH are added to the compound dilution before the microsome mix is added. The final concentration during incubation are: 3 μM test compound or control compound, 0.5 mg/mL microsomes, 0.4 U/mL G6PDH, 3.3 mM MgCl₂, 3.3 mM glucose-6-phosphate and 1.3 mM NaDP+.

After 30 min of incubation, the reaction is stopped with 2 volumes of MeOH.

Of both time points, samples are mixed, centrifuged and the supernatant is harvested for analysis on LC-MS/MS. The instrument responses (i.e. peak heights) are referenced to the zero time-point samples (as 100%) in order to determine the percentage of compound remaining Standard compounds Propanolol and Verapamil are included in the assay design.

The data on microsomal stability are expressed as a percentage of the total amount of compound remaining after 30 min.

### Example 4.8

### Pharmacokinetic Study in Rodents

### 4.8.1 Animals

Sprague-Dawley rats (male, 5-6 weeks old) are obtained from Janvier (France). Rats are acclimatized for at least 7 days before treatment and are kept on a 12 h light/dark cycle (0700-1900). Temperature is maintained at approximately 22° C., and food and water are provided ad libitum. Two days before administration of the test compounds, rats underwent surgery to place a catheter in the jugular vein under isoflurane anesthesia. After the surgery, rats are housed individually. Rats are deprived of food for at least 16 h before oral dosing and 6 h after. Water is provided ad libitum.

### 4.8.2 Pharmacokinetic Study

Compounds are formulated in PEG200/physiological saline (60/40) for the intravenous route and in 0.5% methylcellulose and 10% hydroxylpropyl-P-cyclodextrine pH 3 for the oral route. Test compounds are orally dosed as a single esophageal gavage at 5 mg/kg under a dosing volume of 5 mL/kg and intravenously dosed as a bolus via the caudal vein at 1 mg/kg under a dosing volume of 5 mL/kg. Each group consisted of 3 rats. Blood samples are collected via the jugular vein with lithium heparin as anti-coagulant at the following time points: 0.05, 0.25, 0.5, 1, 3, 5 and 8 h (intravenous route), and 0.25, 0.5, 1, 3, 5, 8 and 24 h (oral route). Alternatively, blood samples are collected at the retro-orbital sinus with lithium heparin as anti-coagulant at the following time points 0.25, 1, 3 and 6 h (oral route). Whole blood samples are centrifuged at 5000 rpm for 10 min and the resulting plasma samples are stored at −20° C. pending analysis.

### 4.8.3 Quantification of Compound Levels in Plasma

Plasma concentrations of each test compound are determined by an LC-MS/MS method in which the mass spectrometer is operated in positive electrospray mode.

### 4.8.4 Determination of Pharmacokinetic Parameters

Pharmacokinetic parameters are calculated using Winnonlin® (Pharsight®, United States).

### Example 4.9

### 7-Day Rat Toxicity Study

A 7-day oral toxicity study with test compounds is performed in Sprague-Dawley male rats to assess their toxic potertial and toxicokinetics, at daily doses of 100, 300 and 500 mg/kg/day, by gavage, at the constant dosage-volume of 5 mL/kg/day.

The test compounds are formulated in 30% (v/v) HPβCD in purified water. Each group included 5 principal male rats as well as 3 satellite animals for toxicokinetics. A fourth group is given 30% (v/v) HPβCD in water only, at the same frequency, dosage volume and by the same route of administration, and acted as the vehicle control group.

The goal of the study is to determine the lowest dose that resulted in no adverse events being identified (no observable adverse effect level—NOAEL).

### Example 4.10

### Hepatocyte Stability

Models to evaluate metabolic clearance in hepatocyte are described by McGinnity et al. Drug Metabolism and Disposition 2008, 32, 11, 1247.

### Example 4.11

### Liability for QT Prolongation

Potertial for QT prolongation is assessed in the hERG patch clamp assay.

### Example 4.10

### Conventional Whole-Cell Patch-Clamp

Whole-cell patch-clamp recordings are performed using an EPC10 amplifier controlled by Pulse v8.77 software (HEKA). Series resistance is typically less than 10 MΩ and compensated by greater than 60%, recordings are not leak subtracted. Electrodes are manufactured from GC150TF pipette glass (Harvard).

The external bathing solution contained: 135 mM NaCl, 5 mM KCl, 1.8 mM CaCl₂, 5 mM Glucose, 10 mM HEPES, pH 7.4.

The internal patch pipette solution contained: 100 mM Kgluconate, 20 mM KCl, 1 mM CaCl₂, 1 mM MgCl₂, 5 mM Na₂ATP, 2 mM Glutathione, 11 mM EGTA, 10 mM HEPES, pH 7.2.

Drugs are perfused using a Biologic MEV-9/EVH-9 rapid perfusion system.

All recordings are performed on HEK293 cells stably expressing hERG channels. Cells are cultured on 12 mm round coverslips (German glass, Bellco) anchored in the recording chamber using two platinum rods (Goodfellow). hERG currents are evoked using an activating pulse to +40 mV for 1000 ms followed by a tail current pulse to 50 mV for 2000 ms, holding potertial is −80 mV. Pulses are applied every 20 s and all experiments are performed at room temperature.

It will be appreciated by those skilled in the art that the foregoing descriptions are exemplary and explanatory in nature, and intended to illustrate the invention and its preferred embodiments. Through routine experimentation, an artisan will recognise apparent modifications and variations that may be made without departing from the spirit of the invention. Thus, the invention is intended to be defined not by the above description, but by the following claims and their equivalents.

### REFERENCES

Bundgard, H., 1985 Design of Prodrugs, pp. 7-9, 21-24, Elsevier, Amsterdam.
Bush K. A. et al. 2002 Arthritis Rheum. 46: 802-5.
Choy E. H. et al. 2001 N Engl J. Med. 344: 907-16.
Chubinskaya S. et al. 2003 The international journal of biochemistry & cell biology 35(9)1323-1340.
Clegg D. O. et al. 2006 N Engl J. Med. 2006 354:795-808.
Constantinescu et al. 2007 Trends in Biochemical Sciences 33(3): 122-131.
Firestein G. S. 2003 Nature. 423:356-61.
Geron et al. 2008 Cancer Cell 13 (4), 321-30
Ip et al. 2006 Clin. Exp. Immun, 162-172.
Jou I M, et al. 2005 Arthritis Rheum. 52:339-44.
Khachigian, L. M. et al. 2006 Nature Protocols 1, 2512-6.
Kopf et al. 2010 Nat. Rev. Drug Disc., 703-718.
Kudlacz et al. 2008 Eur J Pharmaco 154-161.
Lee D. M. et al. 2001 Lancet. 358: 903-11.
Legendre F et al. 2003 J Biol. Chem. 278(5)2903-2912.
Levy D. et al. New England Journal of Medicine 2007 357 1655-1658
Li W. Q. et al. 2001 J Immunol 166:3491-3498.
Lin H. S. et al. 2007 Br J. Pharmacol. April; 150 (7):829-31.
McGinnity et al. 2008 Drug Metabolism and Disposition, 32, 11, 1247.
Mullighan C. G. et al. 2009, PNAS 1, 23, 9414-9418.
Nials et al. 2008 Disease Models & Mechanisms, 213-220.
Nishida K et al. 2004 Arthritis Rheum. 10: 3365-76.
O'Shea J. et al. 2004 Nature Review Drug Discovery 3, 555-564.
O'Sullivan et al. 2007 Mol. Immunol. 44(10):2497-506.
O'Dell J R. et al. 2004 N Engl J. Med. 350(25):2591-602.
Osaki Metal. 2003 Biochem J 369:103-115.
Otero Metal. 2005 Arthritis Research & Therapy 7:R581-R591.
Pernis et al. 2002 J. Clin. Invest. 1279.
Rall L C et al. 2004 Rheumatology; 10:1219-23.
Remington's Pharmaceutical Sciences, 17th edition, 1985, Mack Publishing Company, Easton, Pa. 50 2338-2346.
Rodig S. J. et al. 1998 Cell 93: 373-383. Salvemini D et al. 2001 Arthritis Rheum. 44:2909-21.
Shelton D. L. et al. 2005 Pain. 116:8-16.
Sims N A et al. 2004 Arthritis Rheum. 50 2338-2346.
Smolen J. S. et al. 2003. Nat Rev Drug Discov. 2: 473-88.
T. W. Greene and P. G. M. Wuts, 1991 Protecting Groups in Organic Synthesis, Second Edition, Wiley, New York.
Tam, L. et al. 2007 British Journal of Cancer, 97, 378-383.
Tetsuji Naka et al. 2002 Arthritis Res, 4 (suppl 3):5233-5242.
Vainchenker W. et al. 2008 Seminars in Cell & Developmental Biology 19, 385-393.
Vandeghinste et al. WO 2005/124342.
Walsmith J et al. 2004 J. Rheumatol.; 31:23-9.
Wernig et al. 2008 Cancer Cell 13(4), 311-320.
Wieland H A et al. 2005 Nat Rev Drug Discov. 4:331-44.
Wirtz et al. 2007 Advanced Drug Delivery Reviews, 2007, 1073-1083.
Haan et al., 2011 Chem. Biol., 18(3):314-23.
Collins et al., 1992 J Exp Med. 176(4):1043-51.
Reddy et al., 2000 Oncogene 19(21):2532-47.

### FINAL REMARKS

All publications, including but not limited to patents and patent applications, cited in this specification are herein incorporated by reference as if each individual publication were specifically and individually indicated to be incorporated by reference herein as though fully set forth.

From the foregoing description, various modifications and changes in the compositions and methods of this invention will occur to those skilled in the art. All such modifications coming within the scope of the appended claims are intended to be included therein.

It should be understood that factors such as the differential cell penetration capacity of the various compounds can contribute to discrepancies between the activity of the compounds in the in vitro biochemical and cellular assays.

At least some of the chemical names of compounds of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

Chemical structures shown herein were prepared using either ChemDraw® or ISIS®/DRAW. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

1. A compound according to Formula I: 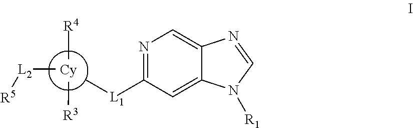
wherein
R¹ is Me, Et, or cyclopropyl, each of which is optionally substituted with one or more halo;
L₁ is —NR²—; —O—, or —CH₂—;
Cy is phenyl, or 5-9 membered heteroaryl comprising 1 to 4 heteroatoms independently selected from N, O, and S;
R² is
C₁₋₂ alkyl optionally substituted with one or more groups independently selected from
C₃₋₇ cycloalkyl,
halo,
CN,
NR^{15a}R^{15b} wherein each R^{15a} and R^{15b} is independently selected from C₁₋₄ alkyl,
4-6 membered heterocycloalkyl containing 1 to 2 heteroatoms independently selected from N, O, and S, and
C₁₋₄ alkoxy, or
C₃₋₇ cycloalkyl;
R³ is
H,
halo,
cyclopropyl,
C₁₋₄ alkyl optionally substituted with one or more halo, or
C₁₋₄ alkoxy optionally substituted with one or more halo;
R⁴ is H, or halo;
L₂ is
absent or is
—W—,
—C₁₋₂ alkylene- (wherein the alkylene is optionally substituted with one CN), or
—C₁₋₂ alkylene-W— (wherein the alkylene is optionally substituted with one CN), or
—CH═CH—;
W is —C(═O)—, —C(═O)O—, —C(═O)NR⁶, —NR⁶C(═O)—, —NR⁶C(═O)O—, —NR⁶C(═O)NH—, —S—, —SO₂—, —SO₂NR⁶, —NHSO₂NR⁶—, —NR⁶SO₂—, —O—, or NR⁶;
R⁵ is:
H,
CN,
C₁₋₆ alkyl optionally substituted with one or more independently selected R⁷ groups,
C₃₋₇ cycloalkyl, optionally substituted with one or more groups independently selected from R¹⁰,
4-7 membered heterocycloalkyl comprising 1 or 2 heteroatoms independently selected from N, O, and S₅ optionally substituted with one or more groups independently selected from R¹⁰,
4-7 membered heterocycloalkenyl comprising 1 double bond, and comprising 1 or 2 heteroatoms independently selected from N, O, and S, optionally substituted with one or more groups independently selected from R¹⁰,
C₆₋₁₀ aryl optionally substituted with one or more groups independently selected from R¹¹, or
5-6 membered heteroaryl comprising 1 to 4 heteroatoms independently selected from N, O, and S, optionally substituted with one or more groups independently selected from R¹¹;
R⁶ is H, or C₁₋₄ alkyl optionally substituted with CN, C₁₋₂ alkoxy, or C₃₋₆ cycloalkyl;
R⁷ is
OH,
CN,
halo,
C₁₋₄ alkoxy,
4-7 membered heterocycloalkyl comprising 1 or 2 heteroatoms independently selected from N, O, and S, optionally substituted with one or more groups independently selected from halo, C₁₋₄ alkyl and oxo,
NR^{8a}R^{8b},
5-6 membered heteroaryl comprising 1 to 4 heteroatoms independently selected from N, O, and S, optionally substituted with one or more independently selected C₁₋₄ alkyl, CN, halo, and C₁₋₄ alkoxy,
phenyl optionally substituted with one or more groups independently selected from C₁₋₄ alkyl, CN, halo, and C₁₋₄ alkoxy,
C₃₋₇ cycloalkyl, or
—C(═O)NR^{9a}R^{9b},
—OSO₂C₁₋₄ alkyl (which alkyl is optionally substituted with one or more halo), or
—NR^{9c}SO₂C₁₋₄ alkyl (which alkyl is optionally substituted with one or more halo);
each R^{8a}, and R^{8b} is independently selected from H, and C₁₋₄ alkyl;
each R^{9a}, R^{9b} and R^{9c} is independently selected from H, and C₁₋₄ alkyl;
each R¹⁰ is independently selected from oxo and R¹¹;
each R¹¹ is halo, —CN or L₃-R¹²;
L₃ is absent or is —C(═O)—, C(═O)O—, —O—, SO₂—, —C(═O)NR^{13a}, —NR^{13b}C(═O), or NR^{13c};
each R¹² is
H,
C₁₋₄ alkyl optionally substituted with one or more independently selected
halo,
OH,
CN,
C₁₋₄ alkoxy,
NHC(═O)O—C₁₋₄ alkyl,
—C(═O)NR^{14a}R^{14b},
—NR^{14c}R^{14d},
—C(═O)C₁₋₄ alkyl,
—C(═O)O—C₁₋₄ alkyl,
phenyl optionally substituted with halo, C₁₋₄ alkyl, C₁₋₄ alkoxy, and
4-7 membered heterocycloalkyl comprising 1 or 2 heteroatoms independently selected from N, O, and S, optionally substituted with one or more C₁₋₄ alkyl,
4-7 membered heterocycloalkyl comprising 1 or 2 heteroatoms independently selected from N, O, and S, optionally substituted with one or more independently selected C₁₋₄ alkyl, oxo and CN,
5-6 membered heteroaryl comprising 1 to 4 heteroatoms independently selected from N, O, and S, optionally substituted with one or more independently selected C₁₋₄ alkyl, and
C₃₋₇ cycloalkyl optionally substituted with one or more independently selected OH, halo, C₁₋₄ alkyl, and CN;
each R^{13a}, R^{13b}, R^{13c}, R^{14a}, R^{14b}, R^{14c}, and R^{14d}, is independently selected from H, and C₁₋₄ alkyl;
provided that R³, R⁴, and -L₂-R⁵ are not all simultaneously H when Cy is C₆ aryl, or 6-membered heteroaryl;
or a pharmaceutically acceptable salt, or a solvate, or a solvate of the pharmaceutically acceptable salt.

2. A compound or pharmaceutically acceptable salt according to claim 1, wherein R¹ is Me or Et.

3. A compound or pharmaceutically acceptable salt according to claim 1, wherein the compound or pharmaceutically acceptable salt is according to Formula IIa or IIb: 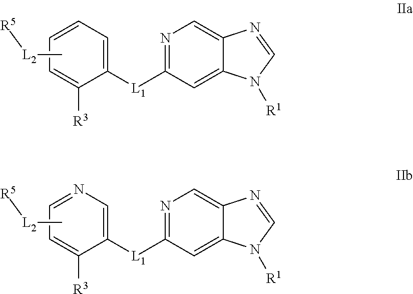
wherein R¹, L₁, R³, L₂, and R⁵ are as described in claim 1.

4. A compound or pharmaceutically acceptable salt according to claim 1, wherein the compound is according to Formula IVa-IVf: 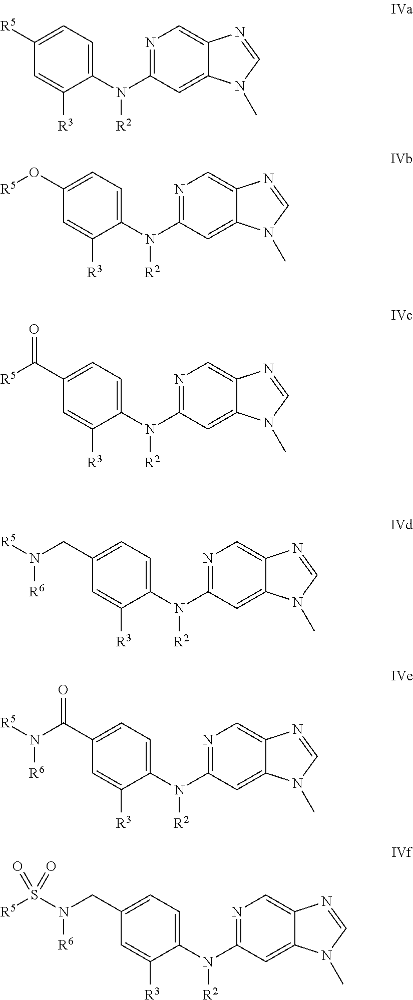
wherein R², R³, R⁶, and R⁵ are as described in claim 1.

5. A compound or pharmaceutically acceptable salt according to claim 4, wherein R³ is C₁₋₄ alkyl.

6. A compound or pharmaceutically acceptable salt according to claim 4,
wherein R² is C₁₋₄ alkyl.

7. A compound or pharmaceutically acceptable salt according to claim 4, wherein the compound is according to Formula VId, VIe, or VIf, and R⁶ is H, Me or Et.

8. A compound or pharmaceutically acceptable salt according to claim 1, wherein R⁵ is 4-7 membered heterocycloalkenyl comprising 1 double bond, and comprising 1 or 2 heteroatoms independently selected from N, O, and S, substituted with one or more groups independently selected from R¹⁰.

9. A compound or pharmaceutically acceptable salt according to claim 1, wherein R⁵ is according to Formula V: 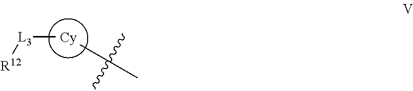
wherein Cy₂ is selected from
C₃₋₇ cycloalkyl,
4-7 membered heterocycloalkyl comprising 1 or 2 heteroatoms independently selected from N, O, and S,
4-7 membered heterocycloalkenyl comprising 1 double bond, and comprising 1 or 2 heteroatoms independently selected from N, O, and S,
C₆₋₁₀ aryl, and
5-6 membered heteroaryl comprising 1 to 4 heteroatoms independently selected from N, O, and S;
L₃ and R¹² are as described in claim 1.

10. A compound or pharmaceutically acceptable salt according to claim 1, wherein the compound is according to Formula VI: 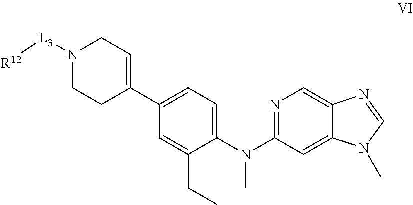
wherein L₃ and R¹² are as described in claim 1.

11. A compound or pharmaceutically acceptable salt according to claim 9, wherein L₃ is —C(═O)—, —C(═O)O—, —O—, or SO₂—.

12. A compound or pharmaceutically acceptable salt according to claim 9, wherein R¹² is Me, Et, n-Pr, i-Pr, or t-Bu.

13. A compound or pharmaceutically acceptable salt according to claim 9, wherein R¹² is —CH₂—CN, —CH₂—CH₂—CN, —CH₂—CH₂—OH—C(OH)H—CH₃, —C(OH)H—CF₃, —CHF₂, —CH₂—CF₃, —CH₂—CMe₂-OH, —CMeH—OMe, —CH₂—OH, CMe₂-OH, —CH₂—OMe, —CH₂—C(═O)t-Bu, —CH₂—C(═O)NH₂, —CH₂-(1-methyloxetan-3-yl), benzyl, —CH₂-4-fluorophenyl, —CH₂-4-chlorophenyl, —CH₂-4-methylphenyl, —CH₂—CH₂—(N-pyrrolidinyl), or —CH₂—CH₂—NMe₂.

14. A pharmaceutical composition comprising a pharmaceutically acceptable carrier and a pharmaceutically effective amount of a compound according to claim 1.

15. The pharmaceutical composition according to claim 14 comprising a further therapeutic agent.

16. (canceled)

17. (canceled)

18. A method for the treatment, or prophylaxis of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons, comprising administering an amount of a compound according to claim 1, sufficient to effect said treatment, or prophylaxis.

19. The method according to claim 18, wherein the compound is administered in combination with a further therapeutic agent.

20. A method for the treatment, or prophylaxis of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons, comprising administering an amount of a pharmaceutical composition according to claim 14, sufficient to effect said treatment, or prophylaxis.

21. The method according to claim 20, wherein the pharmaceutical composition is administered in combination with a further therapeutic agent.
